(19) United States
(12) Patent Application Publication
(10) Pub. No.: US 2012/0065196 A1
(43) Pub. Date: Mar. 15, 2012

(54) AMIDE COMPOUNDS

(76) Inventors: Shuji Kitamura, Osaka (JP); Thomas Daniel Aicher, Superior, CO (US); Steve Gonzales, Media, PA (US); Yvan Le Huerou, Boulder, CO (US); Scott Alan Pratt, Longmont, CO (US); Yoshihisa Nakada, Osaka (JP)

(21) Appl. No.: 12/309,480

(22) PCT Filed: Jul. 20, 2007
(86) PCT No.: PCT/US07/16424

§ 371 Date: May 14, 2009

Related U.S. Application Data
(60) Provisional application No. 60/832,113, filed on Jul. 21, 2006.

(51) Int. Cl. A61K 31/541; A61K 31/454; A61K 31/445; C07D 401/12; C07D 413/12; A61K 31/4245; C07D 513/00; A61K 31/428; A61K 31/497; C07D 403/12; A61K 31/5377; C07D 417/12; A61P 3/04; A61P 3/10; A61P 3/06; C07D 401/06

(52) U.S. Cl. 514/227.8; 546/210; 514/326; 514/318; 546/208; 548/144; 514/364; 548/132; 548/153; 514/367; 514/254.05; 544/371; 544/140; 514/236.5; 544/58.2

(57) ABSTRACT

The present invention provides compounds represented by the formula (Ie):

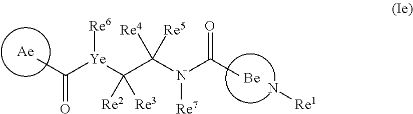

and the formula (If):

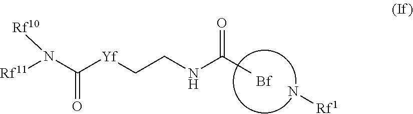

wherein each symbol is as defined in the specification.

According to the present invention, these compounds have a DGAT inhibitory activity and are useful for the prophylaxis, treatment or improvement of diseases or pathologies caused by high expression or high activation of DGAT.

### TECHNICAL FIELD OF THE INVENTION

The present invention relates to a novel amide compound having a diacylglycerol acyl transferase (hereinafter sometimes to be abbreviated as DGAT in the present specification) inhibitory activity, which is useful for the treatment of obesity, hyperlipidemia, diabetes and the like.

### BACKGROUND OF THE INVENTION

Obesity is a state of excess accumulation of fat, mainly triglyceride, in the body, and is deeply involved in the progression into the pathology such as arteriosclerosis, diabetes, hypertension and the like. Therefore, the development of a drug for the prophylaxis or treatment thereof has been desired. In mammals, two major triglyceride synthesis pathways have been biochemically clarified. One is the glycelophosphoric acid pathway present in all tissues, and the other pathway is a monoglyceride pathway. In any pathway, fatty acid in the cell is converted to acyl coenzyme A by an acyl coenzyme A synthetase and introduced into triglyceride through the both pathways. As the enzyme involved in the final stage of the intracellular or intraorgan triglyceride synthesis process, DGAT has been known. As DGAT, DGAT1 and DGAT2 have been cloned. DGAT1 knockout mice have been created and analyzed. As a result, the mice did not become obese easily with high fat diet and showed promoted energy consumption and insulin sensitivity, as compared to wild-type mice. In a mating test of DGAT1 knockout mice and Ay/a mice, moreover, body weight gain was suppressed with a normal diet and a phenotype of promoted insulin sensitivity and elimination of leptin resistance was shown. Thus, DGAT1 inhibitors are expected to be antiobesity drugs.

DGAT is an enzyme (EC2.3.1.20) also designated as acyl coenzyme A:diacylglycerol acyl transferase. cDNA cloning of DGAT1 is reported in Proc. Natl. Acad. Sci. USA. 95, 13018-13023, 1998, and cDNA cloning of DGAT2 is reported in The Journal of Biological Chemistry, 276, 42, 38862-38869, 2001 and The Journal of Biological Chemistry, 276, 42, 38870-38876, 2001. Since the enzyme molecule of DGAT was not clarified for a long time, there is not much finding relating to the DGAT activity. Since the DGAT activity is detected in the endoplasmic reticulum membrane fraction, it was considered to be an endoplasmic reticulum membrane protein. However, ever since cDNA cloning of DGAT was reported, the properties thereof have been rapidly elucidated. For example, it has been reported to be a protein forming a tetramer in Biochem. Journal, 359, 707-714, 2001. A knockout mouse of DGAT1 (DGAT1 defective mouse) was created and its phenotype was reported in Nature Genetics, 25, 87-90, 2000, The Journal of Clinical Investigation, 109, 175-181, 2002 and The Journal of Clinical Investigation, 109, 1049-1055, 2002. From these reports, the DGAT1 inhibitors have been suggested to show an antiobesity action, an anti-insulin resistance action, and an anti-leptin resistance action, and DGAT1 inhibitors are expected to become pharmaceutical products.

In addition, DGAT2 knockout mice were also created and their phenotype is reported in The Journal of Biological Chemistry, 279, 11767-11776 (2004). As a result, DGAT2 was clarified to be an enzyme that plays a key role in the synthesis of triglyceride in the liver. The Journal of Biological Chemistry, 274, 35577-35582, 1999 reports that there are a DGAT activity involved in a storage-type triglyceride synthesis in the cytoplasm side of the endoplasmic reticulum membrane and a DGAT activity that supplies triglyceride to be mobilized for lipoprotein secretion in the lumen side, which suggests that DGAT1 and DGAT2 play different roles as triglyceride synthases and further that DGAT2 inhibitors are effective for hypertriglyceridemia.

In addition, since DGAT expression is promoted in various pathologies and diseases such as obesity, diabetes, insulin-resistant diabetes, leptin resistance, arteriosclerosis, hypertriglyceridemia, hypercholesterolemia, hypertension and the like, high expression or hyper activation of DGAT is suggested to be involved in the excess accumulation of triglyceride in the cell, tissue or organ, and closely involved in the onset and aggravation of these diseases.

In, for example, fat organs and adipocytes, expression of DGAT is regulated by hormones such as insulin, leptin and the like, and DGAT is suggested to be deeply involved in the pathologies such as insulin resistance, leptin resistance and the like. Therefrom it is considered that a compound having a DGAT inhibitory activity is effective for the treatment of obesity, insulin resistant diabetes, hyperorexia or obesity based on leptin resistance.

As amide compounds, the following compounds are known.

(1) A compound represented by the following formula, which is useful as a VLA-1 integrin antagonist (WO2005/016883):

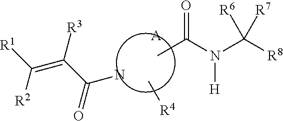

wherein:

A forms, together with the nitrogen atom bonded thereto, a 4-8 membered heterocyclic group which is optionally substituted;

R¹ is aryl, substituted aryl, heteroaryl, substituted heteroaryl, or the like;

R² and R³ are each independently H, alkyl or the like;

R⁴ is H, alkyl, halo, alkoxy, hydroxy, alkylsulfanyl, amino, substituted amino, aminocarbonyl, cyano, or the like;

R⁶ and R⁷ are each independently H, alkyl, substituted alkyl, alkenyl, substituted alkenyl, aryl, substituted aryl, heteroaryl, substituted heteroaryl, heterocyclic, substituted heterocyclic, or the like; and

R⁸ is heteroaryl, substituted heteroaryl, or the like.

(2) Various amide compounds useful as anti-cancer agents (Heterocyclic Communications (1995), 1(5-6), 335-9).
(3) A compound represented by the following formula (I), which is useful as a phosphodiesterase 4 (PDE4) inhibitor (WO2005/116009):

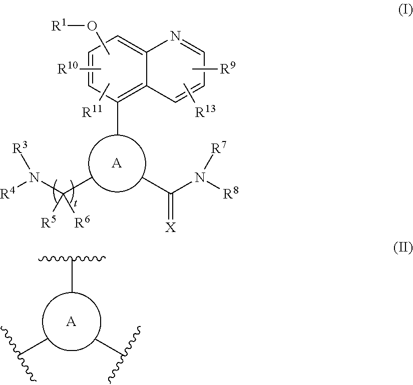

wherein:

formula (II) is a 5-membered heteroaryl;

X is S or O;

R¹ is H, alkyl, cycloalkyl, cycloalkylalkyl-, or the like;

R³ and R⁴ are each independently H, alkyl, hydroxyalkyl or —C(O)O-alkyl;

R⁵ and R⁶ are each independently H, alkyl, hydroxyalkyl, alkoxyalkyl, mercaptoalkyl, or the like;

R⁷ is H, alkyl, alkenyl, hydroxyalkyl, cycloalkyl, alkoxyalkyl, aminoalkyl, (R¹⁷-phenyl)alkyl or —CH₂—C(O)—O-alkyl; and R⁸ is alkyl, heteroaryl, phenyl, cycloalkyl or heterocycloalkyl, all optionally substituted, or a cycloalkyl- or heterocycloalkyl-substituted amide; or R⁷ and R⁸ and the nitrogen to which they are attached together form an optionally substituted ring;

R⁹ is H, halo, alkyl, cycloalkyl, or the like;

R¹⁰, R¹¹ and R¹³ are each independently H or halo; and

R¹⁷ is 1 to 3 substituents independently selected from the group consisting of H, halo, cycloalkyl, and the like.

(4) A compound represented by the following formula, which is an antipicorna virus agent (WO01/96297):

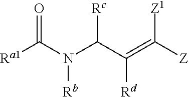

wherein:

R^{a1} is a cycloalkyl, heterocycloalkyl, aryl or heteroaryl group, provided that R^{a1} is not a substituted pyrrolidinyl, where the cycloalkyl, heterocycloalkyl, aryl or heteroaryl group is unsubstituted or substituted with one or more suitable substituents;

R^{c} is a substituent having the formula:

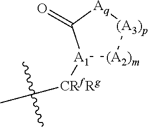

wherein:

R^{f} and R^{g} are each independently H or lower alkyl;

m is 0 or 1;

p is an integer of from 0 to 5;

A₁ is CH or N;

when, p is 1, 2, 3, 4, or 5, A₂ is C(R^{h})(Rⁱ), N(R^{j}) S, S(O), S(O)₂, or O, and when p is O, A₂ is C(R^{h})(Rⁱ)(R^{j}) N(Rⁱ)(R^{j}), S(Rⁱ), S(O)(Rⁱ), S(O)₂(Rⁱ) or O(Rⁱ), where each R^{h}, Rⁱ and R^{j} is independently H or a lower alkyl group;

each A³ is independently C(R^{h})(Rⁱ), N(R^{j}), S, S(O), S(O)₂, or O; where each R^{h}, Rⁱ and R^{j} is independently H or lower alkyl;

when p is 1, 2, 3, 4, or 5, A₄ is N(R^{K})C(R^{h})(Rⁱ), or O; and when p is 0, A₄ is N(R^{k})(R^{l}), C(R^{h})(Rⁱ)(R^{j}) or O(R^{l}), where each R^{h}, Rⁱ and R^{j} is independently H or lower alkyl, each R^{k} is H, alkyl, aryl, or acyl, and each R^{l} is H, alkyl, or aryl;

provided that no more than two heteroatoms occur consecutively in the above-depicted ring formed by A₁, (A₂)ₘ, (A₃)p, A₄, and C═O, where each dotted line in the ring depicts a single bond when A₂ is present and a hydrogen atom when A₂ is absent;

R^{d} is H, halogen, hydroxyl or an alkyl, alkoxy or alkylthio group, where the alkyl, alkoxy or alkylthio group is unsubstituted or substituted with one or more suitable substituents;

R^{b} is H or an alkyl group, unsubstituted or substituted with one or more suitable substituents; and

Z and Z¹ are each independently H, F, an alkyl, cycloalkyl, heterocycloalkyl, aryl or heteroaryl group, where the alkyl, cycloalkyl, heterocycloalkyl, aryl or heteroaryl group is unsubstituted or substituted with one or more suitable substituents, or the like.

(5) A compound represented by the following formula (I), which is useful as a cholecystokinin receptor antagonist (EP697403):

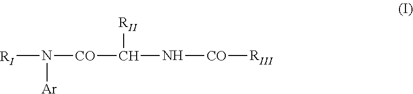

wherein:

R_{I} is C₃₋₈ alkyl, C₁₋₄ alkyl, (C₃₋₁₀)cycloalkyl (C₁₋₄)alkyl, C₃₋₁₀ cycloalkyl, (C₁₋₄)alkoxy(C₂₋₅)alkyl or (CH₂)ᵣCONAB, where A is C₁₋₃ alkyl, B is C₁₋₃ alkyl or phenyl, and r is 1 to 3;

R_{II} is H, C₁₋₆ alkyl, C₁₋₆ hydroxyalkyl, (CH₂)ₘCOR₂, (C₃₋₁₀) cycloalkyl(C₁₋₄)alkyl, C₁₋₄ aminoalkyl or guanidino (C₁₋₄)alkyl, where m is 1 to 3, and R₂ is OH, C₁₋₄ alkoxy or benzyloxy;

R_{III} is naphthyl, quinolyl, isoquinolyl or indolyl; and

Ar is 2-methoxy-3-pyridyl, 4-methoxy-5-pyrimidinyl or 2-methoxyphenyl.

(6) A compound represented by the following formula (1), which is useful as a plant disease control agent (WO96/38419):

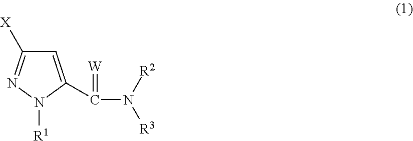

wherein:

X represents a halogen atom;

W represents an oxygen or sulfur atom;

R¹ represents a C₁₋₄ alkyl group; and

R² and R³ each independently represent a hydrogen atom, C₁₋₁₀ alkyl, C₂₋₆ alkenyl, C₂₋₁₀ alkynyl, C₃₋₁₀ cycloalkyl, optionally substituted aryl-C₂₋₄ alkenyl, or the like.

However, none of the above-mentioned prior art reports on the compound of the present invention.

### DISCLOSURE OF THE INVENTION

There is a demand on the development of a novel compound having a superior DGAT inhibitory activity and superior in properties (stability, solubility etc.), oral absorbability, migration to target organs and the like.

The present inventors have searched for a compound having a DGAT inhibitory activity, and found that the compounds represented by the below-mentioned formulas (Ie) and (If) have a superior DGAT inhibitory activity, and are superior in the properties as a pharmaceutical product, such as stability and the like, which resulted in the completion of the present invention.

Accordingly, the present invention relates to

[1] a compound represented by the formula (Ie):

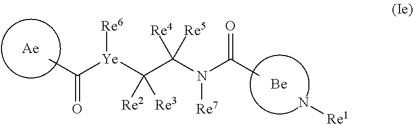

wherein

ring Be is a 5-membered nitrogen-containing aromatic heterocycle optionally condensed with an aromatic ring, which is optionally further substituted;

Re¹ is a substituent;

Ye is CH or N;

ring Ae is an optionally substituted non-aromatic ring; and

Re², Re³, Re⁴, Re⁵, Re⁶ and Re⁷ are each independently a hydrogen atom or a substituent, or any two of Re², Re³, Re⁴, Re⁵, Re⁶ and Re⁷ are optionally bonded to each other to form a non-aromatic ring,

provided that

1) when ring Be is imidazole which is optionally further substituted, then ring Be does not have optionally substituted quinolyl, as a substituent other than Re¹;

2) ring Ae does not have optionally substituted propenoyl as a substituent;

3) when ring Be is pyrrol-2-yl, imidazol-2-yl or pyrazol-5-yl, each of which is optionally further substituted, then Re¹ is an optionally substituted aromatic group;

4) when ring Be is pyrazol-3-yl or pyrazol-4-yl, each of which is optionally further substituted, then Re¹ is not optionally substituted quinolyl; and

5) when ring Be is indole which is optionally further substituted and Ye is CH, then Re¹ is an optionally substituted aromatic group,

or a salt thereof (hereinafter to be abbreviated as compound (Ie));
[2] compound (Ie), wherein ring Be is pyrazole, benzimidazole, indole or indazole, each of which is optionally further substituted;
[3] compound (Ie), wherein Re¹ is an optionally substituted monocyclic aromatic group;
[4] compound (Ie), wherein ring Ae is a non-aromatic ring optionally substituted by 1 to 3 substituents selected from the group consisting of an optionally substituted hydrocarbon group, an optionally substituted heterocyclic group, an optionally substituted hydroxy group, an optionally substituted amino group, an optionally substituted mercapto group, a cyano group, an oxo group, a halogen atom, —CORe^{a1}, —CO—ORe^{a2}, —SO₂Re^{a2} and —CO—NRe^{a′}Re^{b′}
wherein
Re^{a1} is a hydrogen atom, an optionally substituted C₁₋₁₀ alkyl group, an optionally substituted C₃₋₁₀ cycloalkyl group, an optionally substituted C₆₋₁₄ aryl group or an optionally substituted heterocyclic group;
Re^{a2} is a hydrogen atom, an optionally substituted hydrocarbon group or an optionally substituted heterocyclic group; and
Re^{a}′ and Re^{b}′ are each independently a hydrogen atom, an optionally substituted hydrocarbon group or an optionally substituted heterocyclic group, or Re^{a}′ and Re^{b}′ optionally form, together with the adjacent nitrogen atom, an optionally substituted nitrogen-containing heterocycle;
   [5] compound (Ie), wherein all of Re², Re³, Re⁴, Re⁵, Re⁶ and Re⁷ are hydrogen atoms;
   [6] a compound represented by the formula (If):

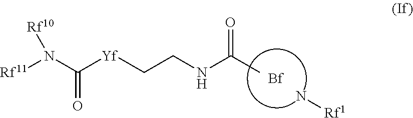

wherein

ring Bf is a 5-membered nitrogen-containing aromatic heterocycle optionally condensed with an aromatic ring, which is optionally further substituted;

Yf is CH₂ or NH;

Rf¹ is a substituent;

Rf¹⁰ is a hydrogen atom or a substituent; and

Rf¹¹ is a hydrogen atom or a C₁₋₆ alkyl group,

provided that

1) when ring Bf is imidazole which is optionally further substituted, then ring Bf does not have optionally substituted quinolyl, as a substituent other than Rf¹;

2) when Yf is CH₂, then ring Bf is not pyrrol-2-yl and imidazol-2-yl, each of which is optionally further substituted;

3) a compound wherein Yf is CH₂, and Rf¹⁰ and Rf¹¹ are hydrogen atoms is excluded;

4) when ring Bf is pyrazol-5-yl which is optionally further substituted, then Rf¹ is an optionally substituted aromatic group; and

5) when ring Bf is pyrazol-3-yl or pyrazol-4-yl, each of which is optionally further substituted, then Rf¹ is not optionally substituted quinolyl,

or a salt thereof (hereinafter to be abbreviated as compound (If));
[7] The compound of above-mentioned [6], wherein ring Bf is pyrazole, benzimidazole, indole or indazole, each of which is optionally further substituted;
[8] The compound of above-mentioned [6], wherein Rf¹ is an optionally substituted C₆₋₁₄ aryl group or an optionally substituted aromatic heterocyclic group;
[9] The compound of above-mentioned [6], wherein Rf¹⁰ is an optionally substituted hydrocarbon group;
[10] N-(2-(1-phenyl-3-(trifluoromethyl)-1H-pyrazole-4-carboxamido)ethyl)-4-propylpiperidine-1-carboxamide (Example E15);
N-(2-(1-phenyl-3-(trifluoromethyl)-1H-pyrazole-4-carboxamido)ethyl)-4-(pyrrolidine-1-carbonyl)piperidine-1-carboxamide (Example E63);
N-(2-(1-phenyl-3-(trifluoromethyl)-1H-pyrazole-4-carboxamido)ethyl)-1-(pyridin-3-ylsulfonyl)piperidine-4-carboxamide (Example E74);
1-phenyl-N-(2-(4-phenylcyclohexanecarboxamido)ethyl)-3-(trifluoromethyl)-1H-pyrazole-4-carboxamide (Example E105);
trans-N-1-isobutyl-N1-methyl-N4-(2-(1-phenyl-3-(trifluoromethyl)-1H-pyrazole-4-carboxamido)ethyl)cyclohexane-1,4-dicarboxamide (Example E126);
N-(2-(trans-4-benzoylcyclohexanecarboxamido)ethyl)-1-(pyridin-2-yl)-3-(trifluoromethyl)-1H-pyrazole-4-carboxamide (Example E150);
N-(2-(trans-4-(5-isopropyl-1,3,4-oxadiazol-2-yl)cyclohexanecarboxamido)ethyl)-1-phenyl-3-(trifluoromethyl)-1H-pyrazole-4-carboxamide (Example E163);
N-(2-(trans-4-benzoylcyclohexanecarboxamido)ethyl)-1-(2-chlorophenyl)-3-(trifluoromethyl)-1H-pyrazole-4-carboxamide (Example E164);
4-(5-isopropyl-1,2,4-oxadiazol-3-yl)-N-(2-(1-phenyl-3-(trifluoromethyl)-1H-pyrazole-4-carboxamido)ethyl)piperidine-1-carboxamide (Example E225);
N-(2-(trans-4-(5-isopropyl-1,2,4-oxadiazol-3-yl)cyclohexanecarboxamido)ethyl)-1-phenyl-3-(trifluoromethyl)-1H-pyrazole-4-carboxamide (Example E228);
2-isopropyl-N-(2-(1-phenyl-3-(trifluoromethyl)-1H-pyrazole-4-carboxamido)ethyl)-4,5,6,7-tetrahydrobenzo[d]thiazole-6-carboxamide (Example E242);
1-(2-chlorophenyl)-N-(2-(trans-4-(5-isopropyl-1,2,4-oxadiazol-3-yl)cyclohexanecarboxamido)ethyl)-3-(trifluoromethyl)-1H-pyrazole-4-carboxamide (Example E257);
1-(2-fluorophenyl)-N-(2-(trans-4-(5-isopropyl-1,2,4-oxadiazol-3-yl)cyclohexanecarboxamido)ethyl)-3-(trifluoromethyl)-1H-pyrazole-4-carboxamide (Example E260);
1-(2-fluorophenyl)-N-(2-(trans-4-(5-isopropyl-1,3,4-oxadiazol-2-yl)cyclohexanecarboxamido)ethyl)-1H-indole-3-carboxamide (Example E265); or
trans-5-(5-isopropyl-1,3,4-oxadiazol-2-yl)-1-methyl-N-(2-(1-phenyl-3-(trifluoromethyl)-1H-pyrazole-4-carboxamido)ethyl)piperidine-2-carboxamide (Example E267); or a salt thereof;
   [11] A prodrug of the compound of above-mentioned [1] or [6];
   [12] A pharmaceutical agent comprising the compound of above-mentioned [1] or [6], or a prodrug thereof;
   [13] The pharmaceutical agent of above-mentioned [12], which is an agent for the prophylaxis or treatment of obesity, hyperlipidemia or diabetes;
   [14] A DGAT inhibitor comprising the compound of above-mentioned [1] or [6], or a prodrug thereof;
[15] Use of the compound of above-mentioned [1] or [6], or a prodrug thereof for the production of an agent for the prophylaxis or treatment of obesity, hyperlipidemia or diabetes;
   [16] Use of the compound of above-mentioned [1] or [6], or a prodrug thereof for the production of a DGAT inhibitor;
   [17] A method for the prophylaxis or treatment of obesity, hyperlipidemia or diabetes in a mammal, which comprises administering the compound of above-mentioned [1] or [6], or a prodrug thereof to the mammal;
   [18] A method of inhibiting DGAT in a mammal, which comprises administering the compound of above-mentioned [1] or [6], or a prodrug thereof to the mammal; and the like.

The compound (Ie) and compound (If) (these are also collectively referred to as the compound of the present invention in this specification) have a DGAT inhibitory activity and are useful for the prophylaxis, treatment or amelioration of diseases or pathologies caused by high expression or high activation of DGAT (sometimes to be abbreviated as DGAT-related diseases in this specification).

### DETAILED DESCRIPTION OF THE INVENTION

In the present specification, unless otherwise specified, the “halogen atom” means fluorine atom, chlorine atom, bromine atom or iodine atom.

In the present specification, unless otherwise specified, the “C₁₋₃ alkylenedioxy group” means methylenedioxy, ethylenedioxy, trimethylenedioxy or the like.

In the present specification, unless otherwise specified, the “C₁₋₆ alkyl group” means methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl or the like.

In the present specification, unless otherwise specified, the “C₁₋₆ alkoxy group” means methoxy, ethoxy, propoxy, isopropoxy, butoxy, isobutoxy, sec-butoxy, tert-butoxy or, the like.

In the present specification, unless otherwise specified, the “C₁₋₆ alkoxy-carbonyl group” means methoxycarbonyl, ethoxycarbonyl, propoxycarbonyl, tert-butoxycarbonyl or the like.

In the present specification, unless otherwise specified, the “C₁₋₆ alkyl-carbonyl group” means acetyl, propanoyl, butanoyl, isobutanoyl, pentanoyl, isopentanoyl, hexanoyl or the like.

Each symbol in the formula (Ie) is described in detail in the following.

In the following explanation, a moiety in the formula (Ie), which is represented by

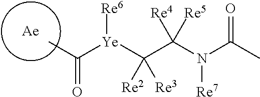

wherein each symbol is as defined in the formula (Ie), is sometimes to be referred to as substituent A.

Re¹ is a substituent.

Re², Re³, Re⁴, Re⁵, Re⁶ and Re⁷ are each independently a hydrogen atom or a substituent, or any two of Re², Re³, Re⁴, Re⁵, Re⁶ and Re⁷ are optionally bonded to each other to form a non-aromatic ring.

As the “substituent” for Re¹, Re², Re³, Re⁴, Re⁵, Re⁶ or Re⁷, an “optionally substituted hydrocarbon group”, an “optionally substituted heterocyclic group”, an “optionally substituted hydroxy group”, an “optionally substituted amino group”, an “optionally substituted mercapto group”, a “cyano group”, a “nitro group”, an “acyl group”, a “halogen atom” and the like can be mentioned.

As the “hydrocarbon group” of the aforementioned “optionally substituted hydrocarbon group”, for example, a C₁₋₁₀ alkyl group, a C₂₋₁₀ alkenyl group, a C₂₋₁₀ alkynyl group, a C₃₋₁₀ cycloalkyl group, a C₃₋₁₀ cycloalkenyl group, a C₉₋₁₀ cycloalkadienyl group, a C₆₋₁₄ aryl group, a C₇₋₁₃ aralkyl group, a C₆₋₁₃ arylalkenyl group, a C₃₋₁₀ cycloalkyl-C₁₋₆ alkyl group and the like can be mentioned.

Here, as the C₁₋₁₀ alkyl group, for example, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, heptyl, octyl, nonyl, decyl and the like can be mentioned.

As the C₂₋₁₀ alkenyl group, for example, ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, 5-hexenyl, 1-heptenyl, 1-octenyl and the like can be mentioned.

As the C₂₋₁₀ alkynyl group, for example, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 1-octynyl and the like can be mentioned.

As the C₃₋₁₀ cycloalkyl group, for example, cyclopropyl, cyclobutyl, cyclopentyl, cyclohexyl, cycloheptyl, cyclooctyl and the like can be mentioned.

As the C₃₋₁₀ cycloalkenyl group, for example, 2-cyclopenten-1-yl, 3-cyclopenten-1-yl, 2-cyclohexen-1-yl, 3-cyclohexen-1-yl and the like can be mentioned.

As the C₄₋₁₀ cycloalkadienyl group, for example, 2,4-cyclopentadien-1-yl, 2,4-cyclohexadien-1-yl, 2,5-cyclohexadien-1-yl and the like can be mentioned.

The above-mentioned C₃₋₁₆ cycloalkyl group, C₃₋₁₀ cycloalkenyl group and C₄₋₁₀ cycloalkadienyl group are each optionally condensed with a benzene ring, and as such a fused ring group, for example, indanyl, dihydronaphthyl, tetrahydronaphthyl, fluorenyl and the like can be mentioned.

The above-mentioned C₃₋₁₀ cycloalkyl group, C₃₋₁₀ cycloalkenyl group and C₄₋₁₀ cycloalkadienyl group each may be a C₇₋₁₀ crosslinked hydrocarbon group. As the C₇₋₁₀ crosslinked hydrocarbon group, bicyclo[2.2.1]heptyl(norbornyl), bicyclo[2.2.2]octyl, bicyclo[3.2.1]octyl, bicyclo[3.2.2]nonyl, bicyclo[3.3.1]nonyl, bicyclo[4.2.1]nonyl, bicyclo[4.3.1]decyl, adamantyl and the like can be mentioned.

The above-mentioned C₃₋₁₀ cycloalkyl group, C₃₋₁₀ cycloalkenyl group and C₄₋₁₀ cycloalkadienyl group each optionally form, together with a C₃₋₁₀ cycloalkane, a C₃₋₁₀ cycloalkene or a C₄₋₁₀ cycloalkadiene, a spiro ring group. Here, as the C₃₋₁₀ cycloalkane, C₃₋₁₀ cycloalkene and C₄₋₁₀ cycloalkadiene, rings corresponding to the above-mentioned C₃₋₁₀ cycloalkyl group, C₃₋₁₀ cycloalkenyl group and C₄₋₁₀ cycloalkadienyl group can be mentioned. As such a spiro ring group, spiro[4.5]decan-8-yl and the like can be mentioned.

As the C₆₋₁₄ aryl group, for example, phenyl, naphthyl, anthryl, phenanthryl, acenaphthylenyl, biphenylyl and the like can be mentioned.

As the C₇₋₁₃ aralkyl group, for example, benzyl, phenethyl, naphthylmethyl, biphenylylmethyl and the like can be mentioned.

As the C₈₋₁₃ arylalkenyl group, for example, styryl and the like can be mentioned.

As the C₃₋₁₀ cycloalkyl-C₁₋₆ alkyl group, for example, cyclohexylmethyl and the like can be mentioned.

The C₁₋₁₀ alkyl group, C₂₋₁₀ alkenyl group and C₂₋₁₀ alkynyl group, which are exemplarily recited as the aforementioned “hydrocarbon group”, each optionally have 1 to 3 substituents at substitutable position(s).

As such substituents, for example,

(1) a C₃₋₁₀ cycloalkyl group (e.g., cyclopropyl, cyclohexyl);
(2) a C₈₋₁₄ aryl group (e.g., phenyl, naphthyl) optionally substituted by 1 to 3 substituents selected from

(a) a C₁₋₈ alkyl group optionally substituted by 1 to 3 halogen atoms,

(b) a hydroxy group,

(c) a C₁₋₈ alkoxy group optionally substituted by 1 to 3 halogen atoms,

(d) a halogen atom,

(e) a cyano group, and

(f) a C₁₋₈ alkylsulfonyl group (e.g., methylsulfonyl);

(3) an aromatic heterocyclic group (e.g., thienyl, furyl, pyridyl, pyrazolyl, imidazolyl, tetrazolyl, oxazolyl, thiazolyl, oxadiazolyl, thiadiazolyl, benzothiazolyl, pyrazinyl, quinolyl, indolyl, pyrimidinyl, triazolyl, isoxazolyl) optionally substituted by 1 to 3 substituents selected from

(a) a halogen atom,

(b) a hydroxy group,

(c) a C₁₋₈ alkyl group optionally substituted by 1 to 3 substituents selected from
(i) a halogen atom,
(ii) a C₁₋₈ alkoxy group optionally substituted by 1 to 3 halogen atoms,
(iii) a C₁₋₈ alkoxy-carbonyl group, and
(iv) an amino group optionally mono- or di-substituted by C₁₋₆ alkyl group(s),

(d) a C₁₋₆ alkoxy group optionally substituted by 1 to 3 halogen atoms,

(e) a C₆₋₁₄ aryl group (e.g., phenyl) optionally substituted by 1 to 3 substituents selected from
(i) a halogen atom,
(ii) a C₁₋₆ alkyl group optionally substituted by 1 to 3 halogen atoms, and
(iii) a C₁₋₆ alkylsulfonyl group (e.g., methylsulfonyl),

(f) a C₇₋₁₃ aralkyl group (e.g., benzyl),

(g) a C₃₋₁₀ cycloalkyl group (e.g., cyclopropyl) optionally substituted by 1 to 3 halogen atoms,

(h) an aromatic heterocyclic group (e.g., pyridyl, thienyl, pyrimidinyl), and

(i) a non-aromatic heterocyclic group (e.g., tetrahydropyranyl);

(4) a non-aromatic heterocyclic group (e.g., tetrahydrofuryl, morpholinyl, thiomorpholinyl, piperidinyl, pyrrolidinyl, piperazinyl, dioxolyl, dioxolanyl, 1,3-dihydro-2-benzofuranyl, thiazolidinyl, tetrahydropyranyl, dihydrooxadiazolyl) optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group optionally substituted by 1 to 3 halogen atoms,

(d) an oxo group, and

(e) a halogen atom;

(5) an amino group optionally mono- or di-substituted by substituent(s) selected from

(a) a C₁₋₆ alkyl group optionally substituted by 1 to 3 halogen atoms,

(b) a C₁₋₆ alkyl-carbonyl group optionally substituted by 1 to 3 halogen atoms,

(c) a C₁₋₆ alkoxy-carbonyl group optionally substituted by 1 to 3 halogen atoms,

(d) a C₆₋₁₄ aryl-carbonyl group (e.g., benzoyl) optionally substituted by 1 to 3 C₁₋₆ alkyl groups optionally substituted by 1 to 3 halogen atoms,

(e) a C₇₋₁₃ aralkyl-carbonyl group (e.g., benzylcarbonyl) optionally substituted by 1 to 3 halogen atoms,

(f) a C₃₋₁₀ cycloalkyl-carbonyl group (e.g., cyclopropylcarbonyl, cyclohexylcarbonyl) optionally substituted by 1 to 3 halogen atoms,

(g) an aromatic heterocyclylcarbonyl group (e.g., pyrazolylcarbonyl, pyrazinylcarbonyl, isoxazolylcarbonyl, pyridylcarbonyl) optionally substituted by 1 to 3 C₁₋₆ alkyl groups optionally substituted by 1 to 3 halogen atoms,

(h) a non-aromatic heterocyclylcarbonyl group (e.g., tetrahydrofurylcarbonyl, tetrahydrothiopyranylcarbonyl),

(i) a C₁₋₆ alkylsulfonyl group (e.g., methylsulfonyl),

(j) a C₆₋₁₄ arylsulfonyl group (e.g., benzenesulfonyl),

(k) an aromatic heterocyclylsulfonyl group (e.g., thienylsulfonyl),

(l) a C₃₋₁₀ cycloalkyl group (e.g., cyclopropyl) optionally substituted by 1 to 3 halogen atoms,

(m) a C₆₋₁₄ aryl group (e.g., phenyl) optionally substituted by 1 to 3 C₁₋₆ alkyl groups optionally substituted by 1 to 3 halogen atoms, and

(n) an aromatic heterocyclic group (e.g., pyrazolyl, pyrazinyl, isoxazolyl, pyridyl) optionally substituted by 1 to 3 C₁₋₆ alkyl groups optionally substituted by 1 to 3 halogen atoms;

(6) an amidino group;
(7) a C₁₋₆ alkyl-carbonyl group optionally substituted by 1 to 3 halogen atoms;
(8) a C₁₋₆ alkoxy-carbonyl group optionally substituted by 1 to 3 substituents selected from

(a) a halogen atom, and

(b) a C₁₋₆ alkoxy group;

(9) a C₁₋₆ alkylsulfonyl group (e.g., methylsulfonyl, ethylsulfonyl, isopropylsulfonyl) optionally substituted by 1 to 3 halogen atoms;
(10) a C₃₋₁₀ cycloalkylsulfonyl group (e.g., cyclopropylsulfonyl);
(11) a C₆₋₁₄ arylsulfonyl group (e.g., benzenesulfonyl) optionally substituted by 1 to 3 substituents selected from

(a) a C₁₋₆ alkyl group optionally substituted by 1 to 3 halogen atoms, and

(b) a C₁₋₆ alkoxy group optionally substituted by 1 to 3 halogen atoms;

(12) an aromatic heterocyclylsulfonyl group (e.g., imidazolylsulfonyl, pyridylsulfonyl) optionally substituted by 1 to 3 C₁₋₆ alkyl groups;
(13) a carbamoyl group optionally mono- or di-substituted by substituent(s) selected from

(a) a C₁₋₆ alkyl group optionally substituted by 1 to 3 substituents selected from
(i) a halogen atom,
(ii) an aromatic heterocyclic group (e.g., pyridyl, furyl) optionally substituted by 1 to 3 C₁₋₆ alkyl groups, and
(iii) a C₁₋₆ alkylsulfonyl group (e.g., methylsulfonyl),

(b) a C₆₋₁₄ aryl group (e.g., phenyl),

(c) a C₇₋₁₃ aralkyl group (e.g., benzyl),

(d) an aromatic heterocyclic group (e.g., pyridyl, thiadiazolyl, oxadiazolyl) optionally substituted by 1 to 3 C₁₋₆ alkyl groups optionally substituted by 1 to 3 halogen atoms, and

(e) a non-aromatic heterocyclic group (e.g., 1,1-dioxidotetrahydrothienyl);

(14) a thiocarbamoyl group optionally mono- or di-substituted by C₁₋₆ alkyl group(s) optionally substituted by 1 to 3 halogen atoms;
(15) a sulfamoyl group optionally mono- or di-substituted by C₁₋₆ alkyl group(s) optionally substituted by 1 to 3 halogen atoms;
(16) a carboxy group;
(17) a hydroxy group;
(18) a C₁₋₆ alkoxy group optionally substituted by 1 to 3 substituents selected from

(a) a halogen atom,

(b) a carboxy group,

(c) a C₁₋₆ alkoxy group,

(d) a C₁₋₆ alkoxy-carbonyl group,

(e) an amino group optionally mono- or di-substituted by substituent(s) selected from a C₁₋₆ alkyl group and a C₁₋₆ alkoxy-carbonyl group, and

(f) a non-aromatic heterocyclic group (e.g., morpholinyl);

(19) a C₂₋₆ alkenyloxy group (e.g., ethenyloxy) optionally substituted by 1 to 3 halogen atoms;
(20) a C₃₋₁₀ cycloalkyloxy group (e.g., cyclohexyloxy);
(21) a C₇₋₁₃ aralkyloxy group (e.g., benzyloxy);
(22) a C₆₋₁₄ aryloxy group (e.g., phenyloxy, naphthyloxy);
(23) a C₁₋₆ alkyl-carbonyloxy group (e.g., acetyloxy, tert-butylcarbonyloxy);
(24) a C₃₋₁₀ cycloalkyl-oxycarbonyl group (e.g., cyclopentyloxycarbonyl);
(25) a C₆₋₁₄ aryl-carbonyl group (e.g., benzoyl) optionally substituted by 1 to 3 substituents selected from

(a) a halogen atom, and

(b) a C₁₋₆ alkyl group optionally substituted by 1 to 3 halogen atoms;

(26) a non-aromatic heterocyclylcarbonyl group (e.g., pyrrolidinylcarbonyl, morpholinylcarbonyl, 1,1-dioxidothiomorpholinylcarbonyl) optionally substituted by 1 to 3 substituents selected from

(a) a C₆₋₁₄ aryl group (e.g., phenyl), and

(b) a C₁₋₆ alkyl group optionally substituted by 1 to 3 halogen atoms;

(27) a mercapto group;
(28) a C₁₋₆ alkylthio group (e.g., methylthio, ethylthio) optionally substituted by 1 to 3 halogen atoms;
(29) a C₇₋₁₃ aralkylthio group (e.g., benzylthio);
(30) a C₆₋₁₄ arylthio group (e.g., phenylthio, naphthylthio);
(31) a sulfo group;
(32) a cyano group;
(33) an azido group;
(34) a nitro group;
(35) a nitroso group;
(36) a halogen atom;

(37) a C₁₋₆ alkylsulfinyl group (e.g., methylsulfinyl);

(38) a C₁₋₃ alkylenedioxy group;
(39) an aromatic heterocyclylcarbonyl group (e.g., pyrazolylcarbonyl, pyrazinylcarbonyl, isoxazolylcarbonyl, pyridylcarbonyl, thiazolylcarbonyl) optionally substituted by 1 to 3 C₁₋₆ alkyl groups optionally substituted by 1 to 3 halogen atoms; and the like can be mentioned.

The C₃₋₁₀ cycloalkyl group, C₃₋₁₀ cycloalkenyl group, C₄₋₁₀ cycloalkadienyl group, C₆₋₁₄ aryl group, C₇₋₁₃ aralkyl group, C₈₋₁₃ arylalkenyl group and C₃₋₁₀ cycloalkyl-C₁₋₆ alkyl group, which are exemplarily recited as the aforementioned “hydrocarbon group”, each optionally have 1 to 3 substituents at substitutable position(s).

As such substituents, for example,

(1) those exemplarily recited as the substituents of the aforementioned C₁₋₁₀ alkyl group and the like;
(2) a C₁₋₆ alkyl group optionally substituted by 1 to 3 substituents selected from

(a) a halogen atom,

(b) a carboxy group,

(c) a hydroxy group,

(d) a C₁₋₆ alkoxy-carbonyl group,

(e) a C₁₋₆ alkyl-carbonyloxy group (e.g., acetyloxy),

(f) a carbamoyl group,

(g) a cyano group,

(h) a non-aromatic heterocyclic group (e.g., morpholinyl),

(i) a C₁₋₆ alkoxy group optionally substituted by 1 to 3 C₃₋₁₀ cycloalkyl groups (e.g., cyclopropyl), and

(j) an amino group optionally mono- or di-substituted C₁₋₆ alkyl group(s);

(3) a C₂₋₆ alkenyl group (e.g., ethenyl, 1-propenyl) optionally substituted by 1 to 3 substituents selected from

(a) a halogen atom,

(b) a carboxy group,

(c) a C₁₋₆ alkoxy-carbonyl group, and

(d) a carbamoyl group;

(4) a C₇₋₁₃ aralkyl group (e.g., benzyl) optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group, and

(d) a halogen atom;

(5) an oxo group;
and the like can be mentioned.

As the “heterocyclic group” of the aforementioned “optionally substituted heterocyclic group”, an aromatic heterocyclic group and a non-aromatic heterocyclic group can be mentioned.

Here, as the aromatic heterocyclic group, for example, a 5- to 7-membered monocyclic aromatic heterocyclic group containing, as a ring-constituting atom besides carbon atoms, 1 to 4 heteroatoms selected from an oxygen atom, a sulfur atom and a nitrogen atom, and a fused aromatic heterocyclic group can be mentioned. As the fused aromatic heterocyclic group, for example, a group derived from a fused ring wherein a ring constituting such 5- to 7-membered monocyclic aromatic heterocyclic group, and 1 or 2 rings selected from a 5- or 6-membered aromatic heterocycle containing 1 or 2 nitrogen atoms (e.g., pyrrole, imidazole, pyrazole, pyrazine, pyridine, pyrimidine, oxazole, thiazole), a 5-membered aromatic heterocycle containing one sulfur atom (e.g., thiophene) and a benzene ring are condensed, and the like can be mentioned.

As preferable examples of the aromatic heterocyclic group,

monocyclic aromatic heterocyclic groups such as furyl (e.g., 2-furyl, 3-furyl), thienyl (e.g., 2-thienyl, 3-thienyl), pyridyl (e.g., 2-pyridyl, 3-pyridyl, 4-pyridyl), pyrimidinyl (e.g., 2-pyrimidinyl, 4-pyrimidinyl, 5-pyrimidinyl), pyridazinyl (e.g., 3-pyridazinyl, 4-pyridazinyl), pyrazinyl (e.g., 2-pyrazinyl), pyrrolyl (e.g., 1-pyrrolyl, 2-pyrrolyl, 3-pyrrolyl), imidazolyl (e.g., 1-imidazolyl, 2-imidazolyl, 4-imidazoyl, 5-imidazolyl), pyrazolyl (e.g., 1-pyrazolyl, 3-pyrazolyl, 4-pyrazolyl), thiazolyl (e.g., 2-thiazolyl, 4-thiazolyl, 5-thiazolyl), isothiazolyl (e.g., 4-isothiazolyl), oxazolyl (e.g., 2-oxazolyl, 4-oxazolyl, 5-oxazolyl), isoxazolyl, oxadiazolyl (e.g., 1,2,4-oxadiazol-5-yl, 1,3,4-oxadiazol-2-yl), thiadiazolyl (e.g., 1,3,4-thiadiazol-2-yl), triazolyl (e.g., 1,2,4-triazol-1-yl, 1,2,4-triazol-3-yl, 1,2,3-triazol-1-yl, 1,2,3-triazol-2-yl, 1,2,3-triazol-4-yl), tetrazolyl (e.g., tetrazol-1-yl, tetrazol-5-yl), triazinyl (e.g., 1,2,4-triazin-1-yl, 1,2,4-triazin-3-yl, 1,3,5-triazin-1-yl) and the like;
fused aromatic heterocyclic groups such as
quinolyl (e.g., 2-quinolyl, 3-quinolyl, 4-quinolyl, 6-quinolyl), isoquinolyl (e.g., 3-isoquinolyl), quinazolyl (e.g., 2-quinazolyl, 4-quinazolyl), quinoxalyl (e.g., 2-quinoxayl, 6-quinoxalyl), benzofuranyl (e.g., 2-benzofuranyl, 3-benzofuranyl), benzothiophenyl (e.g., 2-benzothiophenyl, 3-benzothiophenyl), benzoxazolyl (e.g., 2-benzoxazolyl), benzisoxazolyl (e.g., 7-benzisoxazolyl), benzothiazolyl (e.g., 2-benzothiazolyl), benzimidazolyl (e.g., benzimidazol-1-yl, benzimidazol-2-yl, benzimidazol-5-yl), benzotriazolyl (e.g., 1H-1,2,3-benzotriazol-5-yl), indolyl (e.g., indol-1-yl, indol-2-yl, indol-3-yl, indol-5-yl), indazolyl (e.g., 1H-indazol-3-yl), pyrrolopyrazinyl (e.g., 1H-pyrrolo[2,3-b]pyrazin-2-yl, 1H-pyrrolo[2,3-b]pyrazin-6-yl), imidazopyridinyl (e.g., 1H-imidazo[4,5-b]pyridin-2-yl, 1H-imidazo[4,5-c]pyridin-2-yl, 2H-imidazo[1,2-a]pyridin-3-yl), imidazopyrazinyl (e.g., 1H-imidazo[4,5-b]pyrazin-2-yl), pyrazolopyridinyl (e.g., 1H-pyrazolo[4,3-c]pyridin-3-yl), pyrazolothienyl (e.g., 2H-pyrazolo[3,4-b]thiophen-2-yl), pyrazolotriazinyl (e.g., pyrazolo[5,1-c][1,2,4]triazin-3-yl) and the like;
and the like can be mentioned.

In the present specification, the “heteroaryl group” has the same meaning as the aromatic heterocyclic group described above.

As the non-aromatic heterocyclic group, for example, a 5- to 7-membered monocyclic non-aromatic heterocyclic group containing, as a ring-constituting atom besides carbon atoms, 1 to 4 heteroatoms selected from an oxygen atom, a sulfur atom and a nitrogen atom, and a fused non-aromatic heterocyclic group can be mentioned. As the fused non-aromatic heterocyclic group, for example, a group derived from a fused ring wherein a ring constituting such 5- to 7-membered monocyclic non-aromatic heterocyclic group, and 1 or 2 rings selected from a 5- or 6-membered aromatic or non-aromatic heterocycle containing 1 or 2 nitrogen atoms (e.g., pyrrole, imidazole, pyrazole, pyrazine, pyridine, pyrimidine, oxazole, thiazole), a 5-membered aromatic or non-aromatic heterocycle containing one sulfur atom (e.g., thiophene) and a benzene ring are condensed, a group obtained by partial saturation of said group, and the like can be mentioned.

As preferable examples of the non-aromatic heterocyclic group, tetrahydrofuryl (e.g., 2-tetrahydrofuryl), pyrrolidinyl (e.g., 1-pyrrolidinyl), 1,1-dioxidotetrahydrothienyl (e.g., 1,1-dioxidotetrahydro-3-thienyl), piperidinyl (e.g., piperidino), morpholinyl (e.g., morpholino), thiomorpholinyl (e.g., thiomorpholino), 1,1-dioxidothiomorpholinyl (e.g., 1,1-dioxidothiomorpholino), piperazinyl (e.g., 1-piperazinyl), hexamethyleneiminyl (e.g., hexamethyleneimin-1-yl), oxazolidinyl (e.g., oxazolidin-3-yl), thiazolidinyl (e.g., thiazolidin-3-yl), imidazolidinyl (e.g., imidazolidin-3-yl), dihydroisoindolyl (e.g., 1,3-dihydro-2H-isoindol-2-yl), dioxolyl (e.g., 1,3-dioxol-4-yl), dioxolanyl (e.g., 1,3-dioxolan-4-yl), dihydrooxadiazolyl (e.g., 4,5-dihydro-1,2,4-oxadiazol-3-yl), thioxooxazolidinyl (e.g., 2-thioxo-1,3-oxazolidin-5-yl), tetrahydropyranyl (e.g., 4-tetrahydropyranyl), tetrahydrothiopyranyl (e.g., 4-tetrahydrothiopyranyl), 1,1-dioxidotetrahydrothiopyranyl (e.g., 1,1-dioxidotetrahydrothiopyran-4-yl), dihydrobenzofuranyl (e.g., 2,3-dihydro-1-benzofuran-5-yl), dihydrobenzodioxinyl (e.g., 2,3-dihydro-1,4-benzodioxin-2-yl), dihydrobenzodioxepinyl (e.g., 3,4-dihydro-2H-1,5-benzodioxepin-2-yl), tetrahydrobenzofuranyl (e.g., 4,5,6,7-tetrahydro-1-benzofuran-3-yl), tetrahydrobenzothiazolyl (e.g., 4,5,6,7-tetrahydro-1-benzothiazol-2-yl, 4,5,6,7-tetrahydro-1-benzothiazol-6-yl), tetrahydrobenzoxazolyl (e.g., 4,5,6,7-tetrahydro-1-benzoxazol-2-yl, 4,5,6,7-tetrahydro-1-benzoxazol-6-yl), tetrahydrobenzimidazolyl (e.g., 4,5,6,7-tetrahydro-1H-benzimidazol-5-yl), chromenyl (e.g., 4H-chromen-2-yl, 2H-chromen-3-yl), dihydroquinolinyl (e.g., 1,2-dihydroquinolin-2-yl), tetrahydroquinolinyl (e.g., 1,2,3,4-tetrahydroquinolin-2-yl), dihydroisoquinolinyl (e.g., 1,2-dihydroisoquinolin-2-yl), tetrahydroisoquinolinyl (e.g., 1,2,3,4-tetrahydroisoquinolin-4-yl, 1,2,3,4-tetrahydroisoquinolin-2-yl), dihydrophthalazinyl (e.g., 1,4-dihydrophthalazin-4-yl), pyrazolidinyl (e.g., pyrazolidin-1-yl), tetrahydroindazolyl (e.g., 4,5,6,7-tetrahydro-2H-indazol-2-yl), tetrahydroquinazolinyl (e.g., 5,6,7,8-tetrahydroquinazolin-6-yl), tetrahydrothiazolopyridinyl (e.g., 4,5,6,7-tetrahydrothiazolo [5.4-c]pyridin-6-yl), tetrahydroimidazopyridinyl (e.g., 1,2,3,4-tetrahydroimidazo[4.5-c]pyridin-2-yl, 5,6,7,8-tetrahydroimidazo[1.2-a]pyridin-6-yl, 5,6,7,8-tetrahydroimidazo[1.2-a]pyridin-7-yl), tetrahydropyrazolopyridinyl (e.g., 1,2,3,4-tetrahydropyrazolo[3.4-c]pyridin-2-yl), tetrahydrotriazolopyrazinyl (e.g., 1,2,3,4-tetrahydrotriazolo[4.3-a]pyrazin-2-yl), tetrahydroimidazopyrazinyl (e.g., 1,2,3,4-tetrahydroimidazo[1.2-a]pyrazin-2-yl, 1,2,3,4-tetrahydroimidazo[3.4-a]pyrazin-2-yl), tetrahydropyridopyrimidinyl (e.g., 5,6,7,8-tetrahydropyrido [5.4-c]pyrimidin-6-yl) and the like can be mentioned.

The non-aromatic heterocyclic group may be a heterospiro ring group. For example, the above-mentioned non-aromatic heterocyclic group optionally forms, together with a C₃₋₁₀ cycloalkane, a C₃₋₁₀ cycloalkene, a C₄₋₁₀ cycloalkadiene or a non-aromatic heterocycle, a spiro ring group. Here, as the C₃₋₁₀ cycloalkane, C₃₋₁₀ cycloalkene and C₄₋₄₀ cycloalkadiene, rings corresponding to the C₃₋₁₀ cycloalkyl group, C₃₋₁₀ cycloalkenyl group and C₄₋₁₀ cycloalkadienyl group, which are exemplarily recited as the “hydrocarbon group” of the above-mentioned “optionally substituted hydrocarbon group”, can be mentioned. As the non-aromatic heterocycle, a ring corresponding to the above-mentioned non-aromatic heterocyclic group can be mentioned. As such a spiro ring group, 2,8-diazaspiro[4.5]decan-8-yl and the like can be mentioned.

The above-mentioned non-aromatic heterocyclic group may be a crosslinked non-aromatic heterocyclic group. As the crosslinked non-aromatic heterocyclic group, 2,5-diazabicyclo[2.2.1]heptan-2-yl and the like can be mentioned.

The “heterocyclic group” of the aforementioned “optionally substituted heterocyclic group” optionally has 1 to 3 substituents at substitutable position(s). As such substituents, for example, those exemplarily recited as the substituents of the C₃₋₁₀ cycloalkyl group and the like exemplarily recited as the “hydrocarbon group” of the aforementioned “optionally substituted hydrocarbon group” can be mentioned.

As the aforementioned “optionally substituted hydroxy group”, for example, a hydroxy group optionally substituted by a substituent selected from a C₁₋₁₀ alkyl group, a C₂₋₁₀ alkenyl group, a C₃₋₁₀ cycloalkyl group, a C₃₋₁₀ cycloalkenyl group, a C₆₋₁₄ aryl group, a C₇₋₁₃ aralkyl group, a C₈₋₁₃ arylalkenyl group, a C₁₋₆ alkyl-carbonyl group, a heterocyclic group and the like, each of which is optionally substituted, can be mentioned.

Here, as the C₁₋₁₀ alkyl group, C₂₋₁₀ alkenyl group, C₃₋₁₀ cycloalkyl group, C₃₋₁₀ cycloalkenyl group, C₆₋₁₄ aryl group, C₇₋₁₃ aralkyl group and C₈₋₁₃ arylalkenyl group, those exemplarily recited as the “hydrocarbon group” of the aforementioned “optionally substituted hydrocarbon group” can be mentioned.

As the heterocyclic group, the “aromatic heterocyclic group” and “non-aromatic heterocyclic group”, which are exemplarily recited as the “heterocyclic group” of the aforementioned “optionally substituted heterocyclic group”, can be mentioned.

The aforementioned C₁₋₁₀ alkyl group, C₂₋₁₀ alkenyl group, C₃₋₁₀ cycloalkyl group, C₃₋₁₀ cycloalkenyl group, C₆₋₁₄ aryl group, C₇₋₁₃ aralkyl group, C₈₋₁₃ arylalkenyl group, C₁₋₆ alkyl-carbonyl group and heterocyclic group each optionally have 1 to 3 substituents at substitutable position(s).

As the substituents of the C₁₋₁₀ alkyl group, C₂₋₁₀ alkenyl group and C₁₋₆ alkyl-carbonyl group, those exemplarily recited as the substituents of the C₁₋₁₀ alkyl group and the like exemplarily recited as the “hydrocarbon group” of the aforementioned “optionally substituted hydrocarbon group” can be mentioned.

As the substituents of the C₃₋₁₀ cycloalkyl group, C₃₋₁₀ cycloalkenyl group, C₆₋₁₄ aryl group, C₇₋₁₃ aralkyl group, C₈₋₁₃ arylalkenyl group and heterocyclic group, those exemplarily recited as the substituents of the C₃₋₁₀ cycloalkyl group and the like exemplarily recited as the “hydrocarbon group” of the aforementioned “optionally substituted hydrocarbon group” can be mentioned.

As the aforementioned “optionally substituted mercapto group”, for example, a mercapto group optionally substituted by a substituent selected from a C₁₋₁₀ alkyl group, a C₂₋₁₀ alkenyl group, a C₃₋₁₀ cycloalkyl group, a C₃₋₁₀ cycloalkenyl group, a C₆₋₁₄ aryl group, a C₇₋₁₃ aralkyl group, a C₈₋₁₃ arylalkenyl group, a C₁₋₆ alkyl-carbonyl group, a heterocyclic group and the like, each of which is optionally substituted, can be mentioned.

As the substituents, those exemplarily recited as the substituents of the aforementioned “optionally substituted hydroxy group” can be mentioned.

As the aforementioned “optionally substituted amino group”, for example, an amino group optionally mono- or di-substituted by substituent(s) selected from a C₁₋₁₀ alkyl group, a C₂₋₁₀ alkenyl group, a C₃₋₁₀ cycloalkyl group, a C₃₋₁₀ cycloalkenyl group, a C₆₋₁₄ aryl group, a C₇₋₁₃ aralkyl group, a C₈₋₁₃ arylalkenyl group and a heterocyclic group, each of which is optionally substituted; an acyl group and the like, can be mentioned.

Here, as the C₁₋₁₀ alkyl group, C₂₋₁₀ alkenyl group, C₃₋₁₀ cycloalkyl group, C₃₋₁₀ cycloalkenyl group, C₆₋₁₄ aryl group, C₇₋₁₃ aralkyl group and C₆₋₁₃ arylalkenyl group, those exemplarily recited as the “hydrocarbon group” of the aforementioned “optionally substituted hydrocarbon group” can be mentioned.

As the heterocyclic group, the “aromatic heterocyclic group” and “non-aromatic heterocyclic group”, which are exemplarily recited as the “heterocyclic group” of the aforementioned “optionally substituted heterocyclic group”, can be mentioned. Of these, a 5- to 7-membered monocyclic aromatic heterocyclic group is preferable.

The aforementioned C₁₋₁₀ alkyl group, C₂₋₁₀ alkenyl group, C₃₋₁₀ cycloalkyl group, C₃₋₁₀ cycloalkenyl group, C₆₋₁₄ aryl group, C₇₋₁₃ aralkyl group, C₈₋₁₃ arylalkenyl group and heterocyclic group each optionally have 1 to 3 substituents at substitutable position(s).

As the substituents of the C₁₋₁₀ alkyl group and C₂₋₁₀ alkenyl group, those exemplarily recited as the substituents of the C₁₋₁₀ alkyl group and the like exemplarily recited as the “hydrocarbon group” of the aforementioned “optionally substituted hydrocarbon group” can be mentioned.

As the substituents of the C₃₋₁₀ cycloalkyl group, C₃₋₁₀ cycloalkenyl group, C₆₋₁₄ aryl group, C₇₋₁₃ aralkyl group, C₈₋₁₃ arylalkenyl group and heterocyclic group, those exemplarily recited as the substituents of the C₃₋₁₀ cycloalkyl group and the like exemplarily recited as the “hydrocarbon group” of the aforementioned “optionally substituted hydrocarbon group” can be mentioned.

As the “acyl group” exemplarily recited as the substituent of the “optionally substituted amino group”, those exemplarily recited as “acyl group” below, which is exemplarily recited as the “substituent” for Re¹, Re², Re³, Re⁴, Re⁵, Re⁶ or Re⁷, can be mentioned.

As the “acyl group” which is exemplarily recited as the “substituent” for Re¹, Re², Re³, Re⁴, Re⁵, Re⁶ or Re⁷, for example, a group represented by the formula: —COR^{A}, —CO—OR^{A}, —SO₃R^{A}, —SO₂R^{A}, —SOR^{A}, —CO—NR^{A′}, R^{B′}, —CS—NR^{A′}R^{B′} or —SO₂NR^{A′}R^{B′} wherein R^{A} is a hydrogen atom, an optionally substituted hydrocarbon group or an optionally substituted heterocyclic group, and R^{A′} and R^{B′} are the same or different and each is a hydrogen atom, an optionally substituted hydrocarbon group or an optionally substituted heterocyclic group, or R^{A′} and R^{B′} optionally form, together with the adjacent nitrogen atom, an optionally substituted nitrogen-containing heterocycle, and the like can be mentioned.

As the “optionally substituted hydrocarbon group” and “optionally substituted heterocyclic group” for R^{A}, R^{A′}, or R^{B′}, those exemplarily recited as the “optionally substituted hydrocarbon group” and “optionally substituted heterocyclic group”, which are those exemplarily recited as the “substituent” for Re¹, Re², Re³, Re⁴, Re⁵, Re⁶ or Re⁷, can be mentioned.

As the “nitrogen-containing heterocycle” of the “optionally substituted nitrogen-containing heterocycle” formed by R^{A′} and R^{B′} together with the adjacent nitrogen atom, for example, a 5- to 7-membered nitrogen-containing heterocycle containing, as a ring-constituting atom besides carbon atoms, at least one nitrogen atom and optionally further containing one or two heteroatoms selected from an oxygen atom, a sulfur atom and a nitrogen atom can be mentioned. As preferable examples of the nitrogen-containing heterocycle, pyrrolidine, imidazolidine, pyrazolidine, piperidine, piperazine, morpholine, thiomorpholine, oxopiperazine and the like can be mentioned.

The nitrogen-containing heterocycle optionally has 1 to 3 (preferably 1 or 2) substituents at substitutable position(s). As such substituents, those exemplarily recited as the substituents of the C₃₋₁₀ cycloalkyl group and the like exemplary recited as the “hydrocarbon group” of the aforementioned “optionally substituted hydrocarbon group” can be mentioned.

As preferable examples of the “acyl group”,

(1) a formyl group;
(2) a carboxy group;
(3) a C₁₋₆ alkyl-carbonyl group optionally substituted by 1 to 3 halogen atoms;
(4) a C₁₋₆ alkoxy-carbonyl group (e.g., methoxycarbonyl, ethoxycarbonyl, propoxycarbonyl, tert-butoxycarbonyl) optionally substituted by 1 to 3 substituents selected from

(a) a halogen atom,

(b) a carboxy group,

(c) a carbamoyl group,

(d) a thiocarbamoyl group,

(e) a C₁₋₆ alkoxy group,

(f) a C₁₋₆ alkoxy-carbonyl group, and

(g) a C₁₋₆ alkyl-carbonyloxy group;

(5) a C₃₋₁₀ cycloalkyl-carbonyl group (e.g., cyclopropylcarbonyl, cyclopentylcarbonyl, cyclohexylcarbonyl);
(6) a C₃₋₁₀ cycloalkyl-oxycarbonyl group (e.g., cyclopentyloxycarbonyl);
(7) a C₆₋₁₄ aryl-carbonyl group (e.g., benzoyl, 1-naphthoyl, 2-naphthoyl) optionally substituted by 1 to 3 substituents selected from

(a) a halogen atom,

(b) a cyano group,

(c) a C₁₋₆ alkyl group optionally substituted by 1 to 3 halogen atoms,

(d) a C₁₋₆ alkoxy group,

(e) a carboxy group,

(f) a C₁₋₆ alkoxy-carbonyl group,

(g) an aromatic heterocyclic group (e.g., tetrazolyl, oxadiazolyl),

(h) a non-aromatic heterocyclic group optionally substituted by 1 to 3 oxo groups (e.g., oxooxadiazolinyl), and

(i) a carbamoyl group;

(8) a C₆₋₁₄ aryloxy-carbonyl group (e.g., phenyloxycarbonyl, naphthyloxycarbonyl) optionally substituted by 1 to 3 substituents selected from

(a) a carboxy group,

(b) a C₁₋₆ alkoxy-carbonyl group, and

(c) a carbamoyl group;

(9) a C₇₋₁₃ aralkyloxy-carbonyl group (e.g., benzyloxycarbonyl, phenethyloxycarbonyl) optionally substituted by 1 to 3 substituents selected from

(a) a carboxy group,

(b) a carbamoyl group,

(c) a thiocarbamoyl group,

(d) a C₁₋₆ alkoxy-carbonyl group,

(e) a halogen atom,

(f) a cyano group,

(g) a nitro group,

(h) a C₁₋₆ alkoxy group,

(i) a C₁₋₆ alkylsulfonyl group, and

(j) a C₁₋₆ alkyl group;

(10) a carbamoyl group optionally mono- or di-substituted by substituent(s) selected from

(a) a alkyl group optionally substituted by 1 to 3 substituents selected from
(i) a halogen atom,
(ii) an aromatic heterocyclic group (e.g., pyridyl, furyl) optionally substituted by 1 to 3 C₁₋₆ alkyl groups, and
(iii) a C₁₋₆ alkylsulfonyl group (e.g., methylsulfonyl),

(b) a C₆₋₁₄ aryl group (e.g., phenyl) optionally substituted by 1 to 3 substituents selected from
(i) a halogen atom,
(ii) an aromatic heterocyclic group (e.g., pyridyl, furyl) optionally substituted by 1 to 3 C₁₋₆ alkyl groups, and
(iii) a C₁₋₆ alkylsulfonyl group (e.g., methylsulfonyl),

(c) a C₇₋₁₃ aralkyl group (e.g., benzyl),

(d) an aromatic heterocyclic group (e.g., pyridyl, thiadiazolyl, oxadiazolyl) optionally substituted by 1 to 3 C₁₋₆ alkyl groups optionally substituted by 1 to 3 halogen atoms, and

(e) a non-aromatic heterocyclic group (e.g., 1,1-dioxidotetrahydrothienyl);

(11) a C₁₋₆ alkylsulfonyl group (e.g., methylsulfonyl, ethylsulfonyl, isopropylsulfonyl) optionally substituted by 1 to 3 substituents selected from

(a) a halogen atom,

(b) a carboxy group,

(c) a carbamoyl group, and

(d) a C₁₋₆ alkoxy-carbonyl group;

(12) a C₃₋₁₀ cycloalkylsulfonyl group (e.g., cyclopropyl sulfonyl);
(13) a C₆₋₁₄ arylsulfonyl group (e.g., benzenesulfonyl) optionally substituted by 1 to 3 substituents selected from

(a) a C₁₋₆ alkyl group optionally substituted by 1 to 3 halogen atoms, and

(b) a C₁₋₆ alkoxy group optionally substituted by 1 to 3 halogen atoms;

(14) an aromatic heterocyclylsulfonyl group (e.g., thienylsulfonyl, imidazolylsulfonyl, pyridylsulfonyl) optionally substituted by 1 to 3 C₁₋₆ alkyl groups;
(15) a sulfamoyl group;
(16) a C₁₋₆ alkylsulfinyl group (e.g., methylsulfinyl);
(17) a thiocarbamoyl group;
(18) a C₇₋₁₃ aralkyl-carbonyl group (e.g., benzylcarbonyl, phenethylcarbonyl) optionally substituted by 1 to 3 halogen atoms;
(19) an aromatic heterocyclylcarbonyl group (e.g., furylcarbonyl, thienylcarbonyl, thiazolylcarbonyl, pyrazolylcarbonyl, isoxazolylcarbonyl, pyridylcarbonyl, pyrazinylcarbonyl, benzofurylcarbonyl, benzothienylcarbonyl, quinoxalinylcarbonyl, imidazolylcarbonyl) optionally substituted by 1 to 3 substituents selected from

(a) a C₁₋₆ alkyl group optionally substituted by 1 to 3 halogen atoms,

(b) a C₆₋₁₄ aryl group,

(c) a C₇₋₁₃ aralkyl group,

(d) a C₁₋₆ alkoxy group,

(e) a carboxy group,

(f) a C₁₋₆ alkoxy-carbonyl group, and

(g) a carbamoyl group;

(20) a non-aromatic heterocyclylcarbonyl group (e.g., tetrahydrofurylcarbonyl, tetrahydrothiopyranylcarbonyl, pyrrolidinylcarbonyl, morpholinylcarbonyl, 1,1-dioxidothiomorpholinylcarbonyl) optionally substituted by 1 to 3 substituents selected from

(a) a C₆₋₁₄ aryl group (e.g., phenyl), and

(b) a C₁₋₆ alkyl group optionally substituted by 1 to 3 halogen atoms;

and the like can be mentioned.

As the “non-aromatic ring” formed by any two of Re², Re³, Re⁴, Re⁵, Re⁶ and Re⁷ bonded to each other, a non-aromatic cyclic hydrocarbon and a non-aromatic heterocycle can be mentioned.

Here, as the non-aromatic cyclic hydrocarbon, for example, a C₃₋₁₀ cycloalkane, C₃₋₁₀ cycloalkene, C₄₋₁₀ cycloalkadiene and the like, each of which is optionally condensed with a benzene ring, can be mentioned. As the C₃₋₁₀ cycloalkane, C₃₋₁₀ cycloalkene and C₄₋₁₀ cycloalkadiene, rings corresponding to the C₃₋₁₀ cycloalkyl group, C₃₋₁₀ cycloalkenyl group and C₄₋₁₀ cycloalkadienyl group, which are exemplarily recited as the “substituent” for Re¹, Re², Re³, Re⁴, Re⁵, Re⁶ or Re⁷, can be mentioned.

As the non-aromatic heterocycle, a ring corresponding to the non-aromatic heterocyclic group, which is exemplarily recited as the “substituent” for Re¹, Re², Re³, Re⁴, Re⁵, Re⁶ or Re⁷, can be mentioned.

Re¹ is preferably an optionally substituted cyclic group (a cyclic hydrocarbon group such as a C₆₋₁₄ aryl group, a C₃₋₁₀ cycloalkyl group optionally condensed with a benzene ring, a C₃₋₁₀ cycloalkenyl group optionally condensed with a benzene ring, a C₄₋₁₀ cycloalkadienyl group optionally condensed with a benzene ring, and the like; or a heterocyclic group), more preferably an optionally substituted C₆₋₁₄ aryl group or an optionally substituted aromatic heterocyclic group, further more preferably an optionally substituted monocyclic aromatic group (preferably, a phenyl group or a monocyclic aromatic heterocyclic group (the monocyclic aromatic heterocyclic group is preferably a 5- or 6-membered monocyclic aromatic heterocyclic group (preferably, pyridyl, pyrimidinyl, imidazolyl, pyrazolyl))).

Here, as the substituents of the “optionally substituted cyclic group”,

(1) a halogen atom,
(2) a hydroxy group,
(3) a C₁₋₆ alkyl group,
(4) a C₁₋₆ alkoxy group and the like are preferable.

Re¹ is particularly preferably a monocyclic aromatic group (preferably, a phenyl group or a monocyclic aromatic heterocyclic group (the monocyclic aromatic heterocyclic group is preferably a 5- or 6-membered monocyclic aromatic heterocyclic group (preferably, pyridyl, pyrimidinyl, imidazolyl, pyrazolyl))), optionally substituted by 1 to 3 substituents selected from

(1) a halogen atom,
(2) a hydroxy group,
(3) a C₁₋₆ alkyl group, and
(4) a C₁₋₆ alkoxy group.

Re² and Re³ are preferably both hydrogen atoms.

Re⁴ and Re⁵ are preferably both hydrogen atoms.

Re⁶ is preferably a hydrogen atom or an optionally substituted hydrocarbon group, more preferably a hydrogen atom or an optionally substituted C₁₋₁₀ alkyl group, further more preferably a hydrogen atom or an optionally substituted C₁₋₆ alkyl group, still more preferably a hydrogen atom or a C₁₋₆ alkyl group, particularly preferably a hydrogen atom.

Re⁷ is preferably a hydrogen atom.

Ring Ae is an optionally substituted non-aromatic ring.

As the “non-aromatic ring” of the “optionally substituted non-aromatic ring” for ring Ae, a non-aromatic cyclic hydrocarbon and a non-aromatic heterocycle can be mentioned.

Here, as the non-aromatic cyclic hydrocarbon, for example, a C₃₋₁₀ cycloalkane, C₃₋₁₀ cycloalkene, C₄₋₁₀ cycloalkadiene and the like, each of which is optionally condensed with a benzene ring, can be mentioned. As the C₃₋₁₀ cycloalkane, C₃₋₁₀ cycloalkene and C₄₋₁₀ cycloalkadiene, rings corresponding to the C₃₋₁₀ cycloalkyl group, C₃₋₁₀ cycloalkenyl group and C₄₋₁₀ cycloalkadienyl group, which are exemplarily recited as the “substituent” for Re¹, Re², Re³, Re⁴, Re⁵, Re⁶ or Re⁷, can be mentioned. The non-aromatic cyclic hydrocarbon can be bonded to a carbon atom of the adjacent carbonyl group at any bondable position.

As the non-aromatic heterocycle, a ring corresponding to the non-aromatic heterocyclic group, which is exemplarily recited as the “substituent” for Re¹, Re², Re³, Re⁴, Re⁵, Re⁶ or Re⁷, can be mentioned. The non-aromatic heterocycle can be bonded to a carbon atom of the adjacent carbonyl group at any bondable position.

As the “non-aromatic ring” of the “optionally substituted non-aromatic ring” for ring Ae,

a C₃₋₁₀ cycloalkane (preferably, cyclohexane, cyclopentane); a non-aromatic heterocycle (preferably, piperidine, piperazine, pyrrolidine, morpholine, thiomorpholine, 1,1-dioxidothiomorpholine, tetrahydroisoquinoline, tetrahydroindazole, tetrahydrobenzimidazole, tetrahydrobenzothiazole, tetrahydrobenzoxazole, tetrahydroquinazoline, tetrahydrothiazolopyridine, tetrahydroimidazopyridine, tetrahydropyrazolopyridine, tetrahydrotriazolopyrazine, tetrahydroimidazopyrazine, tetrahydropyridopyrimidine);
a heterospiro ring (preferably, 2,8-diazaspiro[4.5]decane); a crosslinked non-aromatic heterocycle (preferably, 2,5-diazabicyclo[2.2.1]heptane);
and the like are preferable.

The “non-aromatic ring” of the “optionally substituted non-aromatic ring” for ring Ae optionally has 1 to 3 substituents at substitutable position(s). As such substituents, for example, those exemplarily recited as the “substituent” for Re¹, Re², Re³, Re⁴, Re⁵, Re⁶ or Re⁷, and an oxo group can be mentioned.

As the substituents of ring Ae,

an optionally substituted hydrocarbon group;
an optionally substituted heterocyclic group;
an optionally substituted hydroxy group;
an optionally substituted amino group;
an optionally substituted mercapto group;
a cyano group;
an oxo group;
a halogen atom;

### a —CORe^{a1};

### a —CO—ORe^{a2};

### a —SO₂Re^{a2};

### a —CO—NRe^{a′}Re^{b′};

wherein
Re^{a1} is a hydrogen atom, an optionally substituted C₁₋₁₀ alkyl group, an optionally substituted C₃₋₁₀ cycloalkyl group, an optionally substituted C₆₋₁₄ aryl group or an optionally substituted heterocyclic group;
Re^{a2} is a hydrogen atom, an optionally substituted hydrocarbon group or an optionally substituted heterocyclic group; and
Re^{a}′ and Re^{b}′ are independently a hydrogen atom, an optionally substituted hydrocarbon group or an optionally substituted heterocyclic group, or Re^{a}′ and Re^{b}′ optionally form, together with the adjacent nitrogen atom, an optionally substituted nitrogen-containing heterocycle,
   and the like are preferable.

Here, as the “C₁₋₁₀ alkyl group” of the “optionally substituted C₁₋₁₀ alkyl group” for Re^{a1}, those exemplarily recited as the “substituent” for Re¹, Re², Re³, Re⁴, Re⁵, Re⁶ or Re⁷ can be mentioned. The “C₁₋₁₀ alkyl group” of the “optionally substituted C₁₋₁₀ alkyl group” for Re^{a1} optionally has 1 to 3 substituents at substitutable position(s). As such substituents, for example, those exemplarily recited as the substituents of the C₁₋₁₀ alkyl group and the like exemplarily recited as the “substituent” for Re¹, Re², Re³, Re⁴, Re⁵, Re⁶ or Re⁷ can be mentioned.

As the “C₃₋₁₀ cycloalkyl group” of the “optionally substituted C₃₋₁₀ cycloalkyl group” for Re^{a1}, those exemplarily recited as the “substituent” for Re¹, Re², Re³, Re⁴, Re⁵, Re⁶ or Re⁷ can be mentioned. The “C₃₋₁₀ cycloalkyl group” of the “optionally substituted C₃₋₁₀ cycloalkyl group” for Re^{a1} optionally has 1 to 3 substituents at substitutable position(s). As such substituents, for example, those exemplarily recited as the substituents of the C₃₋₁₀ cycloalkyl group and the like exemplarily recited as the “substituent” for Re¹, Re², Re³, Re⁴, Re⁵, Re⁶ or Re⁷ can be mentioned.

As the “C₆₋₁₄ aryl group” of the “optionally substituted C₆₋₁₄ aryl group” for Re^{a1}, those exemplarily recited as the “substituent” for Re¹, Re², Re³, Re⁴, Re⁵, Re⁶ or Re⁷ can be mentioned. The “C₆₋₁₄ aryl group” of the “optionally substituted C₆₋₁₄ aryl group” for Re^{a1} optionally has 1 to 3 substituents at substitutable position(s). As such substituents, for example, those exemplarily recited as the substituents of the C₃₋₁₀ cycloalkyl group and the like exemplarily recited as the “substituent” for Re¹, Re², Re³, Re⁴, Re⁵, Re⁶ or Re⁷ can be mentioned.

As the “optionally substituted hydrocarbon group” for Re^{a2}, Re^{a}′ or Re^{b}′, those exemplarily recited as the “optionally substituted hydrocarbon group” exemplarily recited as the “substituent” for Re¹, Re², Re³, Re⁴, Re⁵, Re⁶ or Re⁷ can be mentioned.

As the “optionally substituted heterocyclic group” for Re^{a1}, Re^{a2}, Re^{a}′ or Re^{b}′, those exemplarily recited as the “optionally substituted heterocyclic group” exemplarily recited as the “substituent” for Re¹, Re², Re³, Re⁴, Re⁵, Re⁶ or Re⁷ can be mentioned.

As the “optionally substituted nitrogen-containing heterocycle” formed by Re^{a}′ and Re^{b}′ together with the adjacent nitrogen atom, those exemplarily recited as the “optionally substituted nitrogen-containing heterocycle” formed by R^{A}′ and R^{B}′ together with the adjacent nitrogen atom, in the explanation of the “acyl group” exemplarily recited as the “substituent” for Re¹, Re², Re³, Re⁴, Re⁵, Re⁶ or Re⁷, can be mentioned.

As the substituents of ring Ae,

(1) a hydroxy group;
(2) an oxo group;
(3) a cyano group;
(4) a C₁₋₆ alkyl group optionally substituted by 1 to 3 substituents selected from

(a) a halogen atom, and

(b) a hydroxy group;

(5) a C₆₋₁₄ aryl group (e.g., phenyl) optionally substituted by 1 to 3 substituents selected from

(a) a halogen atom,

(b) a cyano group,

(c) a hydroxy group, and

(d) a C₁₋₆ alkoxy group;

(6) a C₇₋₁₃ aralkyl group (e.g., benzyl) optionally substituted by 1 to 3 hydroxy groups;
(7) an aromatic heterocyclic group (e.g., pyridyl, pyrimidinyl, pyrazolyl, imidazolyl, triazolyl, oxazolyl, thiazolyl, isoxazolyl, oxadiazolyl, thiadiazolyl, benzothiazolyl) optionally substituted by 1 to 3 substituents selected from

(a) a hydroxy group,

(b) a C₁₋₆ alkyl group optionally substituted by 1 to 3 substituents selected from
(i) a halogen atom,
(ii) a C₁₋₆ alkoxy group optionally substituted by 1 to 3 C₃₋₁₀ cycloalkyl groups (e.g., cyclopropyl),
(iii) a C₁₋₆ alkoxy-carbonyl group, and
(iv) an amino group optionally mono- or di-substituted C₁₋₆ alkyl group(s),

(c) a C₆₋₁₄ aryl group (e.g., phenyl) optionally substituted by 1 to 3 substituents selected from
(i) a halogen atom,
(ii) a C₁₋₆ alkyl group optionally substituted by 1 to 3 halogen atoms, and
(iii) a C₁₋₆ alkylsulfonyl group (e.g., methylsulfonyl),

(d) a C₇₋₁₃ aralkyl group (e.g., benzyl),

(e) a C₃₋₁₀ cycloalkyl group (e.g., cyclopropyl) optionally substituted by 1 to 3 halogen atoms,

(f) an aromatic heterocyclic group (e.g., pyridyl, thienyl, pyrimidinyl),

(g) a non-aromatic heterocyclic group (e.g., tetrahydropyranyl),

(h) a halogen atom,

(i) a cyano group, and

(j) a C₁₋₆ alkoxy-carbonyl group;

(8) a non-aromatic heterocyclic group (e.g., pyrrolidinyl, dihydrooxadiazolyl) optionally substituted by 1 to 3 oxo groups;
(9) a C₁₋₆ alkoxy group optionally substituted by 1 to 3 halogen atoms;
(10) a C₆₋₁₄ aryloxy group (e.g., phenoxy);
(11) a C₁₋₆ alkyl-carbonyl group optionally substituted by 1 to 3 halogen atoms;
(12) a C₁₋₆ alkoxy-carbonyl group optionally substituted by 1 to 3 substituents selected from

(a) a halogen atom, and

(b) a C₁₋₆ alkoxy group;

(13) a C₃₋₁₀ cycloalkyl-oxycarbonyl group (e.g., cyclopentyloxycarbonyl);
(14) a C₆₋₁₄ aryl-carbonyl group (e.g., benzoyl) optionally substituted by 1 to 3 substituents selected from

(a) a halogen atom, and

(b) a C₁₋₆ alkyl group optionally substituted by 1 to 3 halogen atoms;

(15) an aromatic heterocyclylcarbonyl group (e.g., pyridylcarbonyl, thiazolylcarbonyl, pyrazolylcarbonyl) optionally substituted by 1 to 3 C₁₋₆ alkyl groups;
(16) a non-aromatic heterocyclylcarbonyl group (e.g., pyrrolidinylcarbonyl, morpholinylcarbonyl, 1,1-dioxidothiomorpholinylcarbonyl) optionally substituted by 1 to 3 substituents selected from

(a) a C₆₋₁₄ aryl group (e.g., phenyl), and

(b) a C₁₋₆ alkyl group optionally substituted by 1 to 3 halogen atoms;

(17) a C₁₋₆ alkylsulfonyl group (e.g., methylsulfonyl, ethylsulfonyl, isopropylsulfonyl) optionally substituted by 1 to 3 halogen atoms;
(18) a C₃₋₁₀ cycloalkylsulfonyl group (e.g., cyclopropylsulfonyl);
(19) a C₆₋₁₄ arylsulfonyl group (e.g., benzenesulfonyl) optionally substituted by 1 to 3 substituents selected from

(a) a C₁₋₆ alkyl group optionally substituted by 1 to 3 halogen atoms, and

(b) a C₁₋₆ alkoxy group optionally substituted by 1 to 3 halogen atoms;

(20) an aromatic heterocyclylsulfonyl group (e.g., imidazolylsulfonyl, pyridylsulfonyl) optionally substituted by 1 to 3 C₁₋₆ alkyl groups;
(21) an amino group optionally mono- or di-substituted by substituent(s) selected from

(a) a C₁₋₆ alkoxy-carbonyl group,

(b) a C₆₋₁₄ aryl-carbonyl group (e.g., benzoyl) optionally substituted by 1 to 3 C₁₋₆ alkyl groups optionally substituted by 1 to 3 halogen atoms,

(c) a C₇₋₁₃ aralkyl-carbonyl group (e.g., benzylcarbonyl) optionally substituted by 1 to 3 halogen atoms,

(d) a C₃₋₁₀ cycloalkyl-carbonyl group (e.g., cyclopropylcarbonyl, cyclohexylcarbonyl),

(e) an aromatic heterocyclylcarbonyl group (e.g., pyrazolylcarbonyl, pyrazinylcarbonyl, isoxazolylcarbonyl, pyridylcarbonyl) optionally substituted by 1 to 3 C₁₋₆ alkyl groups,

(f) a non-aromatic heterocyclylcarbonyl group (e.g., tetrahydrofurylcarbonyl, tetrahydrothiopyranylcarbonyl),

(g) a C₆₋₁₄ arylsulfonyl group (e.g., benzenesulfonyl), and

(h) an aromatic heterocyclylsulfonyl group (e.g., thienylsulfonyl);

(22) a carbamoyl group optionally mono- or di-substituted by substituent(s) selected from

(a) a C₁₋₆ alkyl group optionally substituted by 1 to 3 substituents selected from
(i) a halogen atom,
(ii) an aromatic heterocyclic group (e.g., pyridyl, furyl) optionally substituted by 1 to 3 C₁₋₆ alkyl groups, and
(iii) a C₁₋₆ alkylsulfonyl group (e.g., methylsulfonyl),

(b) a C₆₋₁₄ aryl group (e.g., phenyl),

(c) a C₇₋₁₃ aralkyl group (e.g., benzyl),

(d) an aromatic heterocyclic group (e.g., pyridyl, thiadiazolyl, oxadiazolyl) optionally substituted by 1 to 3 C₁₋₆ alkyl groups optionally substituted by 1 to 3 halogen atoms, and

(e) a non-aromatic heterocyclic group (e.g., 1,1-dioxidotetrahydrothienyl);

(23) a halogen atom;
(24) a carboxy group;
and the like are more preferable.

Ring Ae is preferably a non-aromatic ring optionally substituted 1 to 3 substituents selected from

an optionally substituted hydrocarbon group;
an optionally substituted heterocyclic group;
an optionally substituted hydroxy group;
an optionally substituted amino group;
an optionally substituted mercapto group;
a cyano group;
an oxo group;
a halogen atom;

### a —COR^{a1};

### a —CO—ORe²;

### a —SO₂Re^{a2}; and

### a —CO—NRe^{a′}Re^{b′};

wherein each symbol is as defined above.

Ring Ae is more preferably a non-aromatic ring (the non-aromatic ring is preferably a C₃₋₁₀ cycloalkane (preferably, cyclohexane, cyclopentane), a non-aromatic heterocycle (preferably, piperidine, piperazine, pyrrolidine, morpholine, thiomorpholine, 1,1-dioxidothiomorpholine, tetrahydroisoquinoline, tetrahydroindazole, tetrahydrobenzimidazole, tetrahydrobenzothiazole, tetrahydrobenzoxazole, tetrahydroquinazoline, tetrahydrothiazolopyridine, tetrahydroimidazopyridine, tetrahydropyrazolopyridine, tetrahydrotriazolopyrazine, tetrahydroimidazopyrazine, tetrahydropyridopyrimidine), a heterospiro ring (preferably, 2,8-diazaspiro[4.5]decane), or a crosslinked non-aromatic heterocycle (preferably, 2,5-diazabicyclo[2.2.1]heptane)) optionally substituted by 1 to 3 substituents selected from

(1) a hydroxy group;
(2) an oxo group;
(3) a cyano group;
(4) a C₁₋₆ alkyl group optionally substituted by 1 to 3 substituents selected from

(a) a halogen atom, and

(b) a hydroxy group;

(5) a C₆₋₁₄ aryl group (e.g., phenyl) optionally substituted by 1 to 3 substituents selected from

(a) a halogen atom,

(b) a cyano group,

(c) a hydroxy group, and

(d) a C₁₋₆ alkoxy group;

(6) a C₇₋₁₃ aralkyl group (e.g., benzyl) optionally substituted by 1 to 3 hydroxy groups;
(7) an aromatic heterocyclic group (e.g., pyridyl, pyrimidinyl, pyrazolyl, imidazolyl, triazolyl, oxazolyl, thiazolyl, isoxazolyl, oxadiazolyl, thiadiazolyl, benzothiazolyl) optionally substituted by 1 to 3 substituents selected from

(a) a hydroxy group,

(b) a C₁₋₆ alkyl group optionally substituted by 1 to 3 substituents selected from
(i) a halogen atom,
(ii) a C₁₋₆ alkoxy group optionally substituted by 1 to 3 C₃₋₁₀ cycloalkyl groups (e.g., cyclopropyl),
(iii) a C₁₋₆ alkoxy-carbonyl group, and
(iv) an amino group optionally mono- or di-substituted C₁₋₆ alkyl group(s),

(c) a C₆₋₁₄ aryl group (e.g., phenyl) optionally substituted by 1 to 3 substituents selected from
(i) a halogen atom,
(ii) a C₁₋₆ alkyl group optionally substituted by 1 to 3 halogen atoms, and
(iii) a C₁₋₆ alkylsulfonyl group (e.g., methylsulfonyl),

(d) a C₇₋₁₃ aralkyl group (e.g., benzyl),

(e) a C₃₋₁₀ cycloalkyl group (e.g., cyclopropyl) optionally substituted by 1 to 3 halogen atoms,

(f) an aromatic heterocyclic group (e.g., pyridyl, thienyl, pyrimidinyl),

(g) a non-aromatic heterocyclic group (e.g., tetrahydropyranyl),

(h) a halogen atom,

(i) a cyano group, and

(j) a C₁₋₆ alkoxy-carbonyl group;

(8) a non-aromatic heterocyclic group (e.g., pyrrolidinyl, dihydrooxadiazolyl) optionally substituted by 1 to 3 oxo groups;
(9) a C₁₋₆ alkoxy group optionally substituted by 1 to 3 halogen atoms;
(10) a C₆₋₁₄ aryloxy group (e.g., phenoxy);
(11) a C₁₋₆ alkyl-carbonyl group optionally substituted by 1 to 3 halogen atoms;
(12) a C₁₋₆ alkoxy-carbonyl group optionally substituted by 1 to 3 substituents selected from

(a) a halogen atom, and

(b) a C₁₋₆ alkoxy group;

(13) a C₃₋₁₀ cycloalkyl-oxycarbonyl group (e.g., cyclopentyloxycarbonyl);
(14) a C₆₋₁₄ aryl-carbonyl group (e.g., benzoyl) optionally substituted by 1 to 3 substituents selected from

(a) a halogen atom, and

(b) a C₁₋₆ alkyl group optionally substituted by 1 to 3 halogen atoms;

(15) an aromatic heterocyclylcarbonyl group (e.g., pyridylcarbonyl, thiazolylcarbonyl, pyrazolylcarbonyl) optionally substituted by 1 to 3 C₁₋₆ alkyl groups;
(16) a non-aromatic heterocyclylcarbonyl group (e.g., pyrrolidinylcarbonyl, morpholinylcarbonyl, 1,1-dioxidothiomorpholinylcarbonyl) optionally substituted by 1 to 3 substituents selected from

(a) a C₆₋₁₄ aryl group (e.g., phenyl), and

(b) a C₁₋₆ alkyl group optionally substituted by 1 to 3 halogen atoms;

(17) a C₁₋₆ alkylsulfonyl group (e.g., methylsulfonyl, ethylsulfonyl, isopropylsulfonyl) optionally substituted by 1 to 3 halogen atoms;
(18) a C₃₋₁₀ cycloalkylsulfonyl group (e.g., cyclopropylsulfonyl);
(19) a C₆₋₁₄ arylsulfonyl group (e.g., benzenesulfonyl) optionally substituted by 1 to 3 substituents selected from

(a) a C₁₋₆ alkyl group optionally substituted by 1 to 3 halogen atoms, and

(b) a C₁₋₆ alkoxy group optionally substituted by 1 to 3 halogen atoms;

(20) an aromatic heterocyclylsulfonyl group (e.g., imidazolylsulfonyl, pyridylsulfonyl) optionally substituted by 1 to 3 C₁₋₆ alkyl groups;
(21) an amino group optionally mono- or di-substituted by substituent(s) selected from

(a) a C₁₋₆ alkoxy-carbonyl group,

(b) a C₆₋₁₄ aryl-carbonyl group (e.g., benzoyl) optionally substituted by 1 to 3 C₁₋₆ alkyl groups optionally substituted by 1 to 3 halogen atoms,

(c) a C₇₋₁₃ aralkyl-carbonyl group (e.g., benzylcarbonyl) optionally substituted by 1 to 3 halogen atoms,

(d) a C₃₋₁₀ cycloalkyl-carbonyl group (e.g., cyclopropylcarbonyl, cyclohexylcarbonyl),

(e) an aromatic heterocyclylcarbonyl group (e.g., pyrazolylcarbonyl, pyrazinylcarbonyl, isoxazolylcarbonyl, pyridylcarbonyl) optionally substituted by 1 to 3 C₁₋₆ alkyl groups,

(f) a non-aromatic heterocyclylcarbonyl group (e.g., tetrahydrofurylcarbonyl, tetrahydrothiopyranylcarbonyl),

(g) a C₆₋₁₄ arylsulfonyl group (e.g., benzenesulfonyl), and

(h) an aromatic heterocyclylsulfonyl group (e.g., thienylsulfonyl);

(22) a carbamoyl group optionally mono- or di-substituted by substituent(s) selected from

(a) a C₁₋₆ alkyl group optionally substituted by 1 to 3 substituents selected from
(i) a halogen atom,
(ii) an aromatic heterocyclic group (e.g., pyridyl, furyl) optionally substituted by 1 to 3 C₁₋₆ alkyl groups, and
(iii) a C₁₋₆ alkylsulfonyl group (e.g., methylsulfonyl),

(b) a C₆₋₁₄ aryl group (e.g., phenyl),

(c) a C₇₋₁₃ aralkyl group (e.g., benzyl),

(d) an aromatic heterocyclic group (e.g., pyridyl, thiadiazolyl, oxadiazolyl) optionally substituted by 1 to 3 C₁₋₆ alkyl groups optionally substituted by 1 to 3 halogen atoms, and

(e) a non-aromatic heterocyclic group (e.g., 1,1-dioxidotetrahydrothienyl);

(23) a halogen atom; and
(24) a carboxy group.

Ring Be is a 5-membered nitrogen-containing aromatic heterocycle optionally condensed with an aromatic ring, which is optionally further substituted.

As the “5-membered nitrogen-containing aromatic heterocycle optionally condensed with an aromatic ring” of the “5-membered nitrogen-containing aromatic heterocycle optionally condensed with an aromatic ring, which is optionally further substituted” for ring Be, a ring corresponding to the 5-membered nitrogen-containing aromatic heterocyclic group, and a ring corresponding to the 5-membered nitrogen-containing aromatic heterocyclic group condensed with an aromatic ring selected from a 5- or 6-membered aromatic heterocycle containing 1 or 2 nitrogen atoms (e.g., pyrrole, imidazole, pyrazole, pyrazine, pyridine, pyrimidine), a 5-membered aromatic heterocycle containing one sulfur atom (e.g., thiophene) and a benzene ring, can be mentioned, from among the aromatic heterocyclic groups exemplarily recited as the “substituent” for Re¹, Re², Re³, Re⁴, Re⁵, Re⁶ or Re⁷. The 5-membered nitrogen-containing aromatic heterocycle optionally condensed with an aromatic ring can be bonded to a carbon atom of the adjacent carbonyl group at any bondable position of the 5-membered ring thereof.

As the “5-membered nitrogen-containing aromatic heterocycle optionally condensed with an aromatic ring” of the “5-membered nitrogen-containing aromatic heterocycle optionally condensed with an aromatic ring, which is optionally further substituted” for ring Be, pyrazole, benzimidazole, indole and indazole are preferable, and pyrazole is particularly preferable.

The “5-membered nitrogen-containing aromatic heterocycle optionally condensed with an aromatic ring” of the “5-membered nitrogen-containing aromatic heterocycle optionally condensed with an aromatic ring, which is optionally further substituted” for ring Be optionally further has 1 to 3 substituents, besides Re¹, at substitutable position(s). As such substituents, for example, those (except an oxo group) exemplarily recited as the substituents of the C₃₋₁₀ cycloalkyl group and the like exemplarily recited as the “substituent” for Re¹, Re², Re³, Re⁴, Re⁵, Re⁶ or Re⁷ can be mentioned.

As the substituents other than Re¹ of ring Be,

(1) a C₁₋₆ alkyl group optionally substituted by 1 to 3 halogen atoms;
(2) a C₆₋₁₄ aryl group;
(3) an amino group optionally mono- or di-substituted by substituent(s) selected from

(a) a C₁₋₆ alkyl group, and

(b) a C₁₋₆ alkoxy-carbonyl group;

(4) a C₁₋₆ alkoxy group;
(5) a C₇₋₁₃ aralkyloxy group
and the like are preferable (a C₁₋₆ alkyl group optionally substituted by 1 to 3 halogen atoms is particularly preferable).

Ring Be is preferably pyrazole, benzimidazole, indole or indazole (particularly preferably, pyrazole), each of which is substituted by Re¹ and optionally further substituted.

Ring Be is more preferably pyrazole, benzimidazole, indole or indazole (particularly preferably, pyrazole), each of which is substituted by Re¹ and optionally further substituted by 1 to 3 substituents selected from

(1) a C₁₋₆ alkyl group optionally substituted by 1 to 3 halogen atoms;
(2) a C₆₋₁₄ aryl group;
(3) an amino group optionally mono- or di-substituted by substituent(s) selected from

(a) a C₁₋₆ alkyl group, and

(b) a C₁₋₆ alkoxy-carbonyl group;

(4) a C₁₋₆ alkoxy group; and
(5) a C₇₋₁₃ aralkyloxy group
(particularly preferably, a C₁₋₆ alkyl group optionally substituted by 1 to 3 halogen atoms).

Ye is CH or N.

Ye is preferably N.

In compound (Ie),

1) when ring Be is imidazole which is optionally further substituted, then ring Be does not have optionally substituted quinolyl, as a substituent other than Re¹;

2) ring Ae does not have optionally substituted propenoyl as a substituent;

3) when ring Be is pyrrol-2-yl, imidazol-2-yl or pyrazol-5-yl, each of which is optionally further substituted (i.e., when ring Be is pyrrole having substituent A at the 2-position, imidazole having substituent A at the 2-position, or pyrazole having substituent A at the 5-position, each of which is optionally further substituted), then Re¹ is an optionally substituted aromatic group;

4) when ring Be is pyrazol-3-yl or pyrazol-4-yl, each of which is optionally further substituted (i.e., ring Be is pyrazole having substituent A at the 3-position, or pyrazole having substituent A at the 4-position, each of which is optionally further substituted), then Re¹ is not optionally substituted quinolyl; and

5) when ring Be is indole which is optionally further substituted and Ye is CH, then Re¹ is an optionally substituted aromatic group.

As preferable examples of compound (Ie), the following compounds can be mentioned.

### [Compound Ie-A]

A compound wherein

ring Be is pyrazole, benzimidazole, indole or indazole (particularly preferably, pyrazole), each of which is substituted by Re¹ and optionally further substituted

[ring Be is preferably, pyrazole, benzimidazole, indole or indazole (particularly preferably, pyrazole), each of which is substituted by Re¹ and optionally further substituted by 1 to 3 substituents selected from
(1) a C₁₋₆ alkyl group optionally substituted by 1 to 3 halogen atoms;
(2) a C₆₋₁₄ aryl group;
(3) an amino group optionally mono- or di-substituted by substituent(s) selected from

(a) a alkyl group, and

(b) a C₁₋₆ alkoxy-carbonyl group;

(4) a C₁₋₆ alkoxy group; and
(5) a C₇₋₁₃ aralkyloxy group
(particularly preferably, a C₁₋₆ alkyl group optionally substituted by 1 to 3 halogen atoms));

Re¹ is an optionally substituted cyclic group (a cyclic hydrocarbon group such as a C₆₋₁₄ aryl group, a C₃₋₁₀ cycloalkyl group optionally condensed with a benzene ring, a C₃₋₁₀ cycloalkenyl group optionally condensed with a benzene ring, a C₄₋₁₀ cycloalkadienyl group optionally condensed with a benzene ring, and the like; or a heterocyclic group)

[Re¹ is preferably an optionally substituted C₆₋₁₄ aryl group or an optionally substituted aromatic heterocyclic group, more preferably an optionally substituted monocyclic aromatic group (preferably, a phenyl group or a monocyclic aromatic heterocyclic group (the monocyclic aromatic heterocyclic group is preferably a 5- or 6-membered monocyclic aromatic heterocyclic group (preferably, pyridyl, pyrimidinyl, imidazolyl, pyrazolyl))),
particularly preferably a monocyclic aromatic group (preferably, a phenyl group or a monocyclic aromatic heterocyclic group (the monocyclic aromatic heterocyclic group is preferably a 5- or 6-membered monocyclic aromatic heterocyclic group (preferably, pyridyl, pyrimidinyl, imidazolyl, pyrazolyl))), optionally substituted by 1 to 3 substituents selected from
(1) a halogen atom,
(2) a hydroxy group,
(3) a C₁₋₆ alkyl group, and
(4) a C₁₋₆ alkoxy group];

Ye is CH or N (preferably, N);

ring Ae is a non-aromatic ring optionally substituted 1 to 3 substituents selected from

an optionally substituted hydrocarbon group;
an optionally substituted heterocyclic group;
an optionally substituted hydroxy group;
an optionally substituted amino group;
an optionally substituted mercapto group;
a cyano group;
an oxo group;

### a —CORe^{a1};

### a —CO—ORe^{a2};

### a —SO₂Re^{a2}; and

### a —CO—NRe^{a}'Re^{b′};

wherein
Re^{a1} is a hydrogen atom, an optionally substituted C₁₋₁₀ alkyl group, an optionally substituted C₃₋₁₀ cycloalkyl group, an optionally substituted C₆₋₁₄ aryl group or an optionally substituted heterocyclic group;
Re^{a2} is a hydrogen atom, an optionally substituted hydrocarbon group or an optionally substituted heterocyclic group; and
Re^{a}′ and Re^{b}′ are independently a hydrogen atom, an optionally substituted hydrocarbon group or an optionally substituted heterocyclic group, or Re^{a}′ and Re^{b}′ optionally form, together with the adjacent nitrogen atom, an optionally substituted nitrogen-containing heterocycle
   [ring Ae is preferably a non-aromatic ring (the non-aromatic ring is preferably a C₃₋₁₀ cycloalkane (preferably, cyclohexane), a non-aromatic heterocycle (preferably, piperidine, piperazine, pyrrolidine, morpholine, thiomorpholine, 1,1-dioxidothiomorpholine, tetrahydroisoquinoline, tetrahydroindazole, tetrahydrobenzothiazole, tetrahydrobenzoxazole, tetrahydroquinazoline, tetrahydrothiazolopyridine, tetrahydroimidazopyridine, tetrahydropyrazolopyridine, tetrahydrotriazolopyrazine, tetrahydroimidazopyrazine, tetrahydropyridopyrimidine), a heterospiro ring (preferably, 2,8-diazaspiro[4.5]decane), or a crosslinked non-aromatic heterocycle (preferably, 2,5-diazabicyclo[2.2.1]heptane)) optionally substituted by 1 to 3 substituents selected from
   (1) a hydroxy group;
   (2) an oxo group;
   (3) a cyano group;
   (4) a C₁₋₆ alkyl group optionally substituted by 1 to 3 substituents selected from

(a) a halogen atom, and

(b) a hydroxy group;

(5) a C₆₋₁₄ aryl group (e.g., phenyl) optionally substituted by 1 to 3 substituents selected from

(a) a halogen atom,

(b) a cyano group,

(c) a hydroxy group, and

(d) a C₁₋₆ alkoxy group;

(6) a C₇₋₁₃ aralkyl group (e.g., benzyl) optionally substituted by 1 to 3 hydroxy groups;
(7) an aromatic heterocyclic group (e.g., pyridyl, pyrimidinyl, pyrazolyl, imidazolyl, triazolyl, oxazolyl, thiazolyl, isoxazolyl, oxadiazolyl, thiadiazolyl, benzothiazolyl) optionally substituted by 1 to 3 substituents selected from

(a) a hydroxy group,

(b) a C₁₋₆ alkyl group optionally substituted by 1 to 3 substituents selected from
(i) a halogen atom,
(ii) a C₁₋₆ alkoxy group,
(iii) a C₁₋₆ alkoxy-carbonyl group, and
(iv) an amino group optionally mono- or di-substituted C₁₋₆ alkyl group(s),

(c) a C₆₋₁₄ aryl group (e.g., phenyl) optionally substituted by 1 to 3 substituents selected from
(i) a halogen atom,
(ii) a C₁₋₆ alkyl group optionally substituted by 1 to 3 halogen atoms, and
(iii) a C₁₋₆ alkylsulfonyl group (e.g., methylsulfonyl),

(d) a C₇₋₁₃ aralkyl group (e.g., benzyl),

(e) a C₃₋₁₀ cycloalkyl group (e.g., cyclopropyl) optionally substituted by 1 to 3 halogen atoms,

(f) an aromatic heterocyclic group (e.g., pyridyl, thienyl, pyrimidinyl), and

(g) a non-aromatic heterocyclic group (e.g., tetrahydropyranyl);

(8) a non-aromatic heterocyclic group (e.g., pyrrolidinyl, dihydrooxadiazolyl) optionally substituted by 1 to 3 oxo groups;
(9) a C₁₋₆ alkoxy group optionally substituted by 1 to 3 halogen atoms;
(10) a C₆₋₁₄ aryloxy group (e.g., phenoxy);
(11) a C₁₋₆ alkyl-carbonyl group optionally substituted by 1 to 3 halogen atoms;
(12) a C₁₋₆ alkoxy-carbonyl group optionally substituted by 1 to 3 substituents selected from

(a) a halogen atom, and

(b) a C₁₋₆ alkoxy group;

(13) a C₃₋₁₀ cycloalkyl-oxycarbonyl group (e.g., cyclopentyloxycarbonyl);
(14) a C₆₋₁₄ aryl-carbonyl group (e.g., benzoyl) optionally substituted by 1 to 3 substituents selected from

(a) a halogen atom, and

(b) a C₁₋₆ alkyl group optionally substituted by 1 to 3 halogen atoms;

(15) an aromatic heterocyclylcarbonyl group (e.g., pyridylcarbonyl, thiazolylcarbonyl, pyrazolylcarbonyl) optionally substituted by 1 to 3 C₁₋₆ alkyl groups;
(16) a non-aromatic heterocyclylcarbonyl group (e.g., pyrrolidinylcarbonyl, morpholinylcarbonyl, 1,1-dioxidothiomorpholinylcarbonyl) optionally substituted by 1 to 3 substituents selected from

(a) a C₆₋₁₄ aryl group (e.g., phenyl), and

(b) a C₁₋₆ alkyl group optionally substituted by 1 to 3 halogen atoms;

(17) a C₁₋₆ alkylsulfonyl group (e.g., methylsulfonyl, ethylsulfonyl, isopropylsulfonyl) optionally substituted by 1 to 3 halogen atoms;
(18) a C₃₋₁₀ cycloalkylsulfonyl group (e.g., cyclopropylsulfonyl);
(19) a C₆₋₁₄ arylsulfonyl group (e.g., benzenesulfonyl) optionally substituted by 1 to 3 substituents selected from

(a) a C₁₋₆ alkyl group optionally substituted by 1 to 3 halogen atoms, and

(b) a C₁₋₆ alkoxy group optionally substituted by 1 to 3 halogen atoms;

(20) an aromatic heterocyclylsulfonyl group (e.g., imidazolylsulfonyl, pyridylsulfonyl) optionally substituted by 1 to 3 C₁₋₆ alkyl groups;
(21) an amino group optionally mono- or di-substituted by substituent(s) selected from

(a) a C₁₋₆ alkoxy-carbonyl group,

(b) a C₆₋₁₄ aryl-carbonyl group (e.g., benzoyl) optionally substituted by 1 to 3 C₁₋₆ alkyl groups optionally substituted by 1 to 3 halogen atoms,

(c) a C₇₋₁₃ aralkyl-carbonyl group (e.g., benzylcarbonyl) optionally substituted by 1 to 3 halogen atoms,

(d) a C₃₋₁₀ cycloalkyl-carbonyl group (e.g., cyclopropylcarbonyl, cyclohexylcarbonyl),

(e) an aromatic heterocyclylcarbonyl group (e.g., pyrazolylcarbonyl, pyrazinylcarbonyl, isoxazolylcarbonyl, pyridylcarbonyl) optionally substituted by 1 to 3 C₁₋₆ alkyl groups,

(f) a non-aromatic heterocyclylcarbonyl group (e.g., tetrahydrofurylcarbonyl, tetrahydrothiopyranylcarbonyl),

(g) a C₆₋₁₄ arylsulfonyl group (e.g., benzenesulfonyl), and

(h) an aromatic heterocyclylsulfonyl group (e.g., thienylsulfonyl); and

(22) a carbamoyl group optionally mono- or di-substituted by substituent(s) selected from

(a) a C₁₋₆ alkyl group optionally substituted by 1 to 3 substituents selected from
(i) a halogen atom,
(ii) an aromatic heterocyclic group (e.g., pyridyl, furyl) optionally substituted by 1 to 3 C₁₋₆ alkyl groups, and
(iii) a C₁₋₆ alkylsulfonyl group (e.g., methylsulfonyl),

(b) a C₆₋₁₄ aryl group (e.g., phenyl),

(c) a C₇₋₁₃ aralkyl group (e.g., benzyl),

(d) an aromatic heterocyclic group (e.g., pyridyl, thiadiazolyl, oxadiazolyl) optionally substituted by 1 to 3 C₁₋₆ alkyl groups optionally substituted by 1 to 3 halogen atoms, and

(e) a non-aromatic heterocyclic group (e.g., 1,1-dioxidotetrahydrothienyl)];
Re² and Re³ are both hydrogen atoms;
Re⁴ and Re⁵ are both hydrogen atoms;
Re⁶ is a hydrogen atom or an optionally substituted hydrocarbon group, preferably a hydrogen atom or an optionally substituted C₁₋₁₀ alkyl group, more preferably a hydrogen atom or an optionally substituted C₁₋₆ alkyl group, further more preferably a hydrogen atom or a C₁₋₆ alkyl group, particularly preferably a hydrogen atom; and
Re⁷ is a hydrogen atom.

### [Compound Ie-B]

A compound wherein

ring Be is pyrazole, benzimidazole, indole or indazole (particularly preferably, pyrazole), each of which is substituted by Re¹ and optionally further substituted

[ring Be is preferably, pyrazole, benzimidazole, indole or indazole (particularly preferably, pyrazole), each of which is substituted by Re¹ and optionally further substituted by 1 to 3 substituents selected from
(1) a C₁₋₆ alkyl group optionally substituted by 1 to 3 halogen atoms;
(2) a C₆₋₁₄ aryl group;
(3) an amino group optionally mono- or di-substituted by substituent(s) selected from

(a) a C₁₋₆ alkyl group, and

(b) a C₁₋₆ alkoxy-carbonyl group;

(4) a C₁₋₆ alkoxy group; and
(5) a C₇₋₁₃ aralkyloxy group
(particularly preferably, a C₁₋₆ alkyl group optionally substituted by 1 to 3 halogen atoms)];

Re¹ is an optionally substituted cyclic group (a cyclic hydrocarbon group such as a C₆₋₁₄ aryl group, a C₃₋₁₀ cycloalkyl group optionally condensed with a benzene ring, a C₃₋₁₀ cycloalkenyl group optionally condensed with a benzene ring, a C₄₋₁₀ cycloalkadienyl group optionally condensed with a benzene ring, and the like; or a heterocyclic group)

[Re¹ is preferably an optionally substituted C₆₋₁₄ aryl group or an optionally substituted aromatic heterocyclic group, more preferably an optionally substituted monocyclic aromatic group (preferably, a phenyl group or a monocyclic aromatic heterocyclic group (the monocyclic aromatic heterocyclic group is preferably a 5- or 6-membered monocyclic aromatic heterocyclic group (preferably, pyridyl, pyrimidinyl, imidazolyl, pyrazolyl))),
particularly preferably a monocyclic aromatic group (preferably, a phenyl group or a monocyclic aromatic heterocyclic group (the monocyclic aromatic heterocyclic group is preferably a 5- or 6-membered monocyclic aromatic heterocyclic group (preferably, pyridyl, pyrimidinyl, imidazolyl, pyrazolyl))), optionally substituted by 1 to 3 substituents selected from
(1) a halogen atom,
(2) a hydroxy group,
(3) a alkyl group, and
(4) a C₁₋₆ alkoxy group];

Ye is CH or N (preferably, N);

ring Ae is a non-aromatic ring optionally substituted 1 to 3 substituents selected from

an optionally substituted hydrocarbon group;
an optionally substituted heterocyclic group;
an optionally substituted hydroxy group;
an optionally substituted amino group;
an optionally substituted mercapto group;
a cyano group;
an oxo group;
a halogen atom;

### a —CORe^{a1};

### a —CO—ORe^{a2};

### a —SO₂Re^{a2}; and

### a —CO—NRe^{a′}Re^{b′};

wherein
Re^{a1} is a hydrogen atom, an optionally substituted C₁₋₁₀ alkyl group, an optionally substituted C₃₋₁₀ cycloalkyl group, an optionally substituted C₆₋₁₄ aryl group or an optionally substituted heterocyclic group;
Re^{a2} is a hydrogen atom, an optionally substituted hydrocarbon group or an optionally substituted heterocyclic group; and
Re^{a}′ and Re^{b}′ are independently a hydrogen atom, an optionally substituted hydrocarbon group or an optionally substituted heterocyclic group, or Re^{a}′ and Re^{b}′ optionally form, together with the adjacent nitrogen atom, an optionally substituted nitrogen-containing heterocycle
   [ring Ae is preferably a non-aromatic ring (the non-aromatic ring is preferably a C₃₋₁₀ cycloalkane (preferably, cyclohexane, cyclopentane), a non-aromatic heterocycle (preferably, piperidine, piperazine, pyrrolidine, morpholine, thiomorpholine, 1,1-dioxidothiomorpholine, tetrahydroisoquinoline, tetrahydroindazole, tetrahydrobenzimidazole, tetrahydrobenzothiazole, tetrahydrobenzoxazole, tetrahydroquinazoline, tetrahydrothiazolopyridine, tetrahydroimidazopyridine, tetrahydropyrazolopyridine, tetrahydrotriazolopyrazine, tetrahydroimidazopyrazine, tetrahydropyridopyrimidine), a heterospiro ring (preferably, 2,8-diazaspiro[4.5]decane), or a crosslinked non-aromatic heterocycle (preferably, 2,5-diazabicyclo[2.2.1]heptane)) optionally substituted by 1 to 3 substituents selected from
   (1) a hydroxy group;
   (2) an oxo group;
   (3) a cyano group;
   (4) a C₁₋₆ alkyl group optionally substituted by 1 to 3 substituents selected from

(a) a halogen atom, and

(b) a hydroxy group;

(5) a C₆₋₁₄ aryl group (e.g., phenyl) optionally substituted by 1 to 3 substituents selected from

(a) a halogen atom,

(b) a cyano group,

(c) a hydroxy group, and

(d) a C₁₋₆ alkoxy group;

(6) a C₇₋₁₃ aralkyl group (e.g., benzyl) optionally substituted by 1 to 3 hydroxy groups;
(7) an aromatic heterocyclic group (e.g., pyridyl, pyrimidinyl, pyrazolyl, imidazolyl, triazolyl, oxazolyl, thiazolyl, isoxazolyl, oxadiazolyl, thiadiazolyl, benzothiazolyl) optionally substituted by 1 to 3 substituents selected from

(a) a hydroxy group,

(b) a C₁₋₆ alkyl group optionally substituted by 1 to 3 substituents selected from
(i) a halogen atom,
(ii) a C₁₋₆ alkoxy group optionally substituted by 1 to 3 C₃₋₁₀ cycloalkyl groups (e.g., cyclopropyl),
(iii) a C₁₋₆ alkoxy-carbonyl group, and
(iv) an amino group optionally mono- or di-substituted C₁₋₆ alkyl group(s),

(c) a C₆₋₁₄ aryl group (e.g., phenyl) optionally substituted by 1 to 3 substituents selected from
(i) a halogen atom,
(ii) a C₁₋₆ alkyl group optionally substituted by 1 to 3 halogen atoms, and
(iii) a C₁₋₆ alkylsulfonyl group (e.g., methylsulfonyl),

(d) a C₇₋₁₃ aralkyl group (e.g., benzyl),

(e) a C₃₋₁₀ cycloalkyl group (e.g., cyclopropyl) optionally substituted by 1 to 3 halogen atoms,

(f) an aromatic heterocyclic group (e.g., pyridyl, thienyl, pyrimidinyl),

(g) a non-aromatic heterocyclic group (e.g., tetrahydropyranyl),

(h) a halogen atom,

(i) a cyano group, and

(j) a C₁₋₆ alkoxy-carbonyl group;

(8) a non-aromatic heterocyclic group (e.g., pyrrolidinyl, dihydrooxadiazolyl) optionally substituted by 1 to 3 oxo groups;
(9) a C₁₋₆ alkoxy group optionally substituted by 1 to 3 halogen atoms;
(10) a C₆₋₁₄ aryloxy group (e.g., phenoxy);
(11) a C₁₋₆ alkyl-carbonyl group optionally substituted by 1 to 3 halogen atoms;
(12) a C₁₋₆ alkoxy-carbonyl group optionally substituted by 1 to 3 substituents selected from

(a) a halogen atom, and

(b) a C₁₋₆ alkoxy group;

(13) a C₃₋₁₀ cycloalkyl-oxycarbonyl group (e.g., cyclopentyloxycarbonyl);
(14) a C₆₋₁₄ aryl-carbonyl group (e.g., benzoyl) optionally substituted by 1 to 3 substituents selected from

(a) a halogen atom, and

(b) a C₁₋₆ alkyl group optionally substituted by 1 to 3 halogen atoms;

(15) an aromatic heterocyclylcarbonyl group (e.g., pyridylcarbonyl, thiazolylcarbonyl, pyrazolylcarbonyl) optionally substituted by 1 to 3 C₁₋₆ alkyl groups;
(16) a non-aromatic heterocyclylcarbonyl group (e.g., pyrrolidinylcarbonyl, morpholinylcarbonyl, 1,1-dioxidothiomorpholinylcarbonyl) optionally substituted by 1 to 3 substituents selected from

(a) a C₆₋₁₄ aryl group (e.g., phenyl), and

(b) a C₁₋₆ alkyl group optionally substituted by 1 to 3 halogen atoms;

(17) a C₁₋₆ alkylsulfonyl group (e.g., methylsulfonyl, ethylsulfonyl, isopropylsulfonyl) optionally substituted by 1 to 3 halogen atoms;
(18) a C₃₋₁₀ cycloalkylsulfonyl group (e.g., cyclopropylsulfonyl);
(19) a C₆₋₁₄ arylsulfonyl group (e.g., benzenesulfonyl) optionally substituted by 1 to 3 substituents selected from

(a) a C₁₋₆ alkyl group optionally substituted by 1 to 3 halogen atoms, and

(b) a C₁₋₆ alkoxy group optionally substituted by 1 to 3 halogen atoms;

(20) an aromatic heterocyclylsulfonyl group (e.g., imidazolylsulfonyl, pyridylsulfonyl) optionally substituted by 1 to 3 C₁₋₆ alkyl groups;
(21) an amino group optionally mono- or di-substituted by substituent(s) selected from

(a) a C₁₋₆ alkoxy-carbonyl group,

(b) a C₆₋₁₄ aryl-carbonyl group (e.g., benzoyl) optionally substituted by 1 to 3 C₁₋₆ alkyl groups optionally substituted by 1 to 3 halogen atoms,

(c) a C₇₋₁₃ aralkyl-carbonyl group (e.g., benzylcarbonyl) optionally substituted by 1 to 3 halogen atoms,

(d) a C₃₋₁₀ cycloalkyl-carbonyl group (e.g., cyclopropylcarbonyl, cyclohexylcarbonyl),

(e) an aromatic heterocyclylcarbonyl group (e.g., pyrazolylcarbonyl, pyrazinylcarbonyl, isoxazolylcarbonyl, pyridylcarbonyl) optionally substituted by 1 to 3 C₁₋₆ alkyl groups,

(f) a non-aromatic heterocyclylcarbonyl group (e.g., tetrahydrofurylcarbonyl, tetrahydrothiopyranylcarbonyl),

(g) a C₆₋₁₄ arylsulfonyl group (e.g., benzenesulfonyl), and

(h) an aromatic heterocyclylsulfonyl group (e.g., thienyl sulfonyl);

(22) a carbamoyl group optionally mono- or di-substituted by substituent(s) selected from

(a) a C₁₋₆ alkyl group optionally substituted by 1 to 3 substituents selected from
(i) a halogen atom,
(ii) an aromatic heterocyclic group (e.g., pyridyl, furyl) optionally substituted by 1 to 3 C₁₋₆ alkyl groups, and
(iii) a C₁₋₆ alkylsulfonyl group (e.g., methylsulfonyl),

(b) a C₆₋₁₄ aryl group (e.g., phenyl),

(c) a C₇₋₁₃ aralkyl group (e.g., benzyl),

(d) an aromatic heterocyclic group (e.g., pyridyl, thiadiazolyl, oxadiazolyl) optionally substituted by 1 to 3 C₁₋₆ alkyl groups optionally substituted by 1 to 3 halogen atoms, and

(e) a non-aromatic heterocyclic group (e.g., 1,1-dioxidotetrahydrothienyl);

(23) a halogen atom; and
(24) a carboxy group];

Re² and Re³ are both hydrogen atoms;

Re⁴ and Re⁵ are both hydrogen atoms;

Re⁶ is a hydrogen atom or an optionally substituted hydrocarbon group, preferably a hydrogen atom or an optionally substituted C₁₋₁₀ alkyl group, more preferably a hydrogen atom or an optionally substituted C₁₋₆ alkyl group, further more preferably a hydrogen atom or a C₁₋₆ alkyl group, particularly preferably a hydrogen atom; and

Re⁷ is a hydrogen atom.

Each symbol in the formula (If) is described in detail in the following.

In the following explanation, a moiety in the formula (If), which is represented by

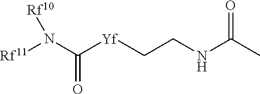

wherein each symbol is as defined in the formula (If), is sometimes to be referred to as substituent B.

Rf¹ is a substituent.

Rf¹⁰ is a hydrogen atom or a substituent.

Re¹¹ is a hydrogen atom or a C₁₋₆ alkyl group.

As the “substituent” for Rf¹ or Rf¹⁰, those exemplarily recited as the “substituent” for Re¹, Re², Re³, Re⁴, Re⁵, Re⁶ or Re⁷ can be mentioned.

Rf¹ is preferably an optionally substituted cyclic group (a cyclic hydrocarbon group such as a C₆₋₁₄ aryl group, a C₃₋₁₀ cycloalkyl group optionally condensed with a benzene ring, a C₃₋₁₀ cycloalkenyl group optionally condensed with a benzene ring, a C₄₋₁₀ cycloalkadienyl group optionally condensed with a benzene ring, and the like; or a heterocyclic group), more preferably an optionally substituted C₆₋₁₄ aryl group or an optionally substituted aromatic heterocyclic group, further more preferably an optionally substituted monocyclic aromatic group (preferably, a phenyl group or a monocyclic aromatic heterocyclic group (the monocyclic aromatic heterocyclic group is preferably a 5- or 6-membered monocyclic aromatic heterocyclic group (preferably, pyridyl, pyrimidinyl, imidazolyl, pyrazolyl))).

As the substituents of Rf¹,

(1) a halogen atom,
(2) a hydroxy group,
(3) a C₁₋₆ alkyl group,
(4) a C₁₋₆ alkoxy group and the like are preferable.

Rf¹ is particularly preferably a monocyclic aromatic group (preferably, a phenyl group or a monocyclic aromatic heterocyclic group (the monocyclic aromatic heterocyclic group is preferably a 5- or 6-membered monocyclic aromatic heterocyclic group (preferably, pyridyl, pyrimidinyl, imidazolyl, pyrazolyl))), optionally substituted by 1 to 3 substituents selected from

(1) a halogen atom,
(2) a hydroxy group,
(3) a C₁₋₆ alkyl group, and
(4) a C₁₋₆ alkoxy group.

Rf¹⁰ is preferably an optionally substituted hydrocarbon group, more preferably an optionally substituted C₁₋₁₀ alkyl group (preferably, a C₁₋₆ alkyl group) or an optionally substituted C₃₋₁₀ cycloalkyl group, particularly preferably a C₁₋₆ alkyl group or a C₃₋₁₀ cycloalkyl group, each of which is optionally substituted by 1 to 3 aromatic heterocyclic groups (preferably, pyridyl, oxadiazolyl) optionally substituted by 1 to 3 C₁₋₆ alkyl groups.

Ring Bf is a 5-membered nitrogen-containing aromatic heterocycle optionally condensed with an aromatic ring, which is optionally further substituted.

As the “5-membered nitrogen-containing aromatic heterocycle optionally condensed with an aromatic ring” of the “5-membered nitrogen-containing aromatic heterocycle optionally condensed with an aromatic ring, which is optionally further substituted” for ring Bf, a ring corresponding to the 5-membered nitrogen-containing aromatic heterocyclic group, and a ring corresponding to the 5-membered nitrogen-containing aromatic heterocyclic group condensed with an aromatic ring selected from a 5- or 6-membered aromatic heterocycle containing 1 or 2 nitrogen atoms (e.g., pyrrole, imidazole, pyrazole, pyrazine, pyridine, pyrimidine), a 5-membered aromatic heterocycle containing one sulfur atom (e.g., thiophene) and a benzene ring, can be mentioned, from among the aromatic heterocyclic groups exemplarily recited as the “substituent” for Re¹, Re², Re³, Re⁴, Re⁵, Re⁶ or Re⁷. The 5-membered nitrogen-containing aromatic heterocycle optionally condensed with an aromatic ring can be bonded to a carbon atom of the adjacent carbonyl group at any bondable position of the 5-membered ring thereof.

As the “5-membered nitrogen-containing aromatic heterocycle optionally condensed with an aromatic ring” of the

“5-membered nitrogen-containing aromatic heterocycle optionally condensed with an aromatic ring, which is optionally further substituted” for ring Bf, pyrazole, benzimidazole, indole and indazole are preferable, and pyrazole is particularly preferable.

The “5-membered nitrogen-containing aromatic heterocycle optionally condensed with an aromatic ring” of the “5-membered nitrogen-containing aromatic heterocycle optionally condensed with an aromatic ring, which is optionally further substituted” for ring Bf optionally further has 1 to 3 substituents, besides Rf¹, at substitutable position(s). As such substituents, for example, those (except an oxo group) exemplarily recited as the substituents of the C₃₋₁₀ cycloalkyl group and the like exemplarily recited as the “substituent” for Re¹, Re², Re³, Re⁴, Re⁵, Re⁶ or Re⁷ can be mentioned.

As the substituents other than Rf¹ of ring Bf,

(1) a C₁₋₆ alkyl group optionally substituted by 1 to 3 halogen atoms;
(2) a C₆₋₁₄ aryl group;
(3) an amino group optionally mono- or di-substituted by substituent(s) selected from

(a) a C₁₋₆ alkyl group, and

(b) a C₁₋₆ alkoxy-carbonyl group;

(4) a C₁₋₆ alkoxy group;
(5) a C₇₋₁₃ aralkyloxy group
and the like are preferable (a C₁₋₆ alkyl group optionally substituted by 1 to 3 halogen atoms is particularly preferable).

Ring Bf is preferably pyrazole, benzimidazole, indole or indazole (particularly preferably, pyrazole), each of which is substituted by Rf¹ and optionally further substituted.

Ring Bf is more preferably pyrazole, benzimidazole, indole or indazole (particularly preferably, pyrazole), each of which is substituted by Rf¹ and optionally further substituted by 1 to 3 substituents selected from

(1) a C₁₋₆ alkyl group optionally substituted by 1 to 3 halogen atoms;
(2) a C₆₋₁₄ aryl group;
(3) an amino group optionally mono- or di-substituted by substituent(s) selected from

(a) a C₁₋₆ alkyl group, and

(b) a C₁₋₆ alkoxy-carbonyl group;

(4) a C₁₋₆ alkoxy group; and
(5) a C₇₋₁₃ aralkyloxy group
(particularly preferably, a C₁₋₆ alkyl group optionally substituted by 1 to 3 halogen atoms).

Yf is CH₂ or NH.

Yf is preferably NH.

In compound (If),

1) when ring Bf is imidazole which is optionally further substituted, then ring Bf does not have optionally substituted quinolyl, as a substituent other than Rf¹;

2) when Yf is CH₂, then ring Bf is not pyrrol-2-yl and imidazol-2-yl, each of which is optionally further substituted (i.e., ring Bf is not pyrrole having substituent B at the 2-position, and imidazole having substituent B at the 2-position, each of which is optionally further substituted);

3) a compound wherein Yf is CH₂, and Rf¹⁰ and Rf¹¹ are hydrogen atoms is excluded;

4) when ring Bf is pyrazol-5-yl which is optionally further substituted (i.e., ring Bf is pyrazole having substituent B at the 5-position, which is optionally further substituted), then Rf¹ is an optionally substituted aromatic group; and

5) when ring Bf is pyrazol-3-yl or pyrazol-4-yl, each of which is optionally further substituted (i.e., ring Bf is pyrazole having substituent B at the 3-position, or pyrazole having substituent B at the 4-position, each of which is optionally further substituted), then Rf¹ is not optionally substituted quinolyl.

As preferable examples of compound (If), the following compounds can be mentioned.

### [Compound If-A]

A compound wherein

ring Bf is pyrazole, benzimidazole, indole or indazole (particularly preferably, pyrazole), each of which is substituted by Rf¹ and optionally further substituted

[ring Bf is preferably, pyrazole, benzimidazole, indole or indazole (particularly preferably, pyrazole), each of which is substituted by Rf¹ and optionally further substituted by 1 to 3 substituents selected from
(1) a C₁₋₆ alkyl group optionally substituted by 1 to 3 halogen atoms;
(2) a C₆₋₁₄ aryl group;
(3) an amino group optionally mono- or di-substituted by substituent(s) selected from

(a) a C₁₋₆ alkyl group, and

(b) a C₁₋₆ alkoxy-carbonyl group;

(4) a C₁₋₆ alkoxy group; and
(5) a C₇₋₁₃ aralkyloxy group
(particularly preferably, a C₁₋₆ alkyl group optionally substituted by 1 to 3 halogen atoms)];

Rf¹ is an optionally substituted cyclic group (a cyclic hydrocarbon group such as a C₆₋₁₄ aryl group, a C₃₋₁₀ cycloalkyl group optionally condensed with a benzene ring, a C₃₋₁₀ cycloalkenyl group optionally condensed with a benzene ring, a C₄₋₁₀ cycloalkadienyl group optionally condensed with a benzene ring, and the like; or a heterocyclic group)

[Rf¹ is preferably an optionally substituted C₆₋₁₄ aryl group or an optionally substituted aromatic heterocyclic group, more preferably an optionally substituted monocyclic aromatic group (preferably, a phenyl group or a monocyclic aromatic heterocyclic group (the monocyclic aromatic heterocyclic group is preferably a 5- or 6-membered monocyclic aromatic heterocyclic group (preferably, pyridyl, pyrimidinyl, imidazolyl, pyrazolyl))),
particularly preferably a monocyclic aromatic group (preferably, a phenyl group or a monocyclic aromatic heterocyclic group (the monocyclic aromatic heterocyclic group is preferably a 5- or 6-membered monocyclic aromatic heterocyclic group (preferably, pyridyl, pyrimidinyl, imidazolyl, pyrazolyl))), optionally substituted by 1 to 3 substituents selected from
(1) a halogen atom,
(2) a hydroxy group,
(3) a C₁₋₆ alkyl group, and
(4) a C₁₋₆ alkoxy group];

Yf is CH₂ or NH (preferably NH);

Rf¹⁰ is an optionally substituted hydrocarbon group

[Rf¹⁰ is preferably an optionally substituted C₁₋₁₀ alkyl group, more preferably an optionally substituted C₁₋₆ alkyl group, particularly preferably a C₁₋₆ alkyl group optionally substituted by 1 to 3 aromatic heterocyclic groups (preferably, pyridyl)]; and

Rf¹¹ is a C₁₋₆ alkyl group.

### [Compound If-B]

A compound wherein

ring Bf is pyrazole, benzimidazole, indole or indazole (particularly preferably, pyrazole), each of which is substituted by Rf¹ and optionally further substituted

[ring Bf is preferably, pyrazole, benzimidazole, indole or indazole (particularly preferably, pyrazole), each of which is substituted by Rf¹ and optionally further substituted by 1 to 3 substituents selected from
(1) a alkyl group optionally substituted by 1 to 3 halogen atoms;
(2) a C₆₋₁₄ aryl group;
(3) an amino group optionally mono- or di-substituted by substituent(s) selected from

(a) a C₁₋₆ alkyl group, and

(b) a C₁₋₆ alkoxy-carbonyl group;

(4) a C₁₋₆ alkoxy group; and
(5) a C₇₋₁₃ aralkyloxy group
(particularly preferably, a C₁₋₆ alkyl group optionally substituted by 1 to 3 halogen atoms)];

Rf¹ is an optionally substituted cyclic group (a cyclic hydrocarbon group such as a C₆₋₁₄ aryl group, a C₃₋₄₀ cycloalkyl group optionally condensed with a benzene ring, a C₃₋₁₀ cycloalkenyl group optionally condensed with a benzene ring, a C₄₋₁₀ cycloalkadienyl group optionally condensed with a benzene ring, and the like; or a heterocyclic group)

[Rf¹ is preferably an optionally substituted C₆₋₁₄ aryl group or an optionally substituted aromatic heterocyclic group, more preferably an optionally substituted monocyclic aromatic group (preferably, a phenyl group or a monocyclic aromatic heterocyclic group (the monocyclic aromatic heterocyclic group is preferably a 5- or 6-membered monocyclic aromatic heterocyclic group (preferably, pyridyl, pyrimidinyl, imidazolyl, pyrazolyl))),
particularly preferably a monocyclic aromatic group (preferably, a phenyl group or a monocyclic aromatic heterocyclic group (the monocyclic aromatic heterocyclic group is preferably a 5- or 6-membered monocyclic aromatic heterocyclic group (preferably, pyridyl, pyrimidinyl, imidazolyl, pyrazolyl))), optionally substituted by 1 to 3 substituents selected from
(1) a halogen atom,
(2) a hydroxy group,
(3) a C₁₋₆ alkyl group, and
(4) a C₁₋₆ alkoxy group];

Yf is CH₂ or NH (preferably NH);

Rf¹⁰ is an optionally substituted hydrocarbon group

[Rf¹⁰ is preferably an optionally substituted C₁₋₁₀ alkyl group (preferably, a C₁₋₆ alkyl group) or an optionally substituted C₃₋₁₀ cycloalkyl group, more preferably a C₁₋₆ alkyl group or a C₃₋₁₀ cycloalkyl group, each of which is optionally substituted by 1 to 3 aromatic heterocyclic groups (preferably, pyridyl, oxadiazolyl) optionally substituted by 1 to 3 C₁₋₆ alkyl groups]; and

Rf¹¹ is a hydrogen atom or a C₁₋₆ alkyl group.

As the other preferable examples of compounds (Ie) and (If), the following compounds can be mentioned.

### [Compound Iz]

N-(2-(1-phenyl-3-(trifluoromethyl)-1H-pyrazole-4-carboxamido)ethyl)-4-propylpiperidine-1-carboxamide (Example E15);
N-(2-(1-phenyl-3-(trifluoromethyl)-1H-pyrazole-4-carboxamido)ethyl)-4-(pyrrolidine-1-carbonyl)piperidine-1-carboxamide (Example E63);
N-(2-(1-phenyl-3-(trifluoromethyl)-1H-pyrazole-4-carboxamido)ethyl)-1-(pyridin-3-ylsulfonyl)piperidine-4-carboxamide (Example E74);
1-phenyl-N-(2-(4-phenylcyclohexanecarboxamido)ethyl)-3-(trifluoromethyl)-1H-pyrazole-4-carboxamide (Example E105);
trans-N1-isobutyl-N-1-methyl-N4-(2-(1-phenyl-3-(trifluoromethyl)-1H-pyrazole-4-carboxamido)ethyl)cyclohexane-1,4-dicarboxamide (Example E126);
N-(2-(trans-4-benzoylcyclohexanecarboxamido)ethyl)-1-(pyridin-2-yl)-3-(trifluoromethyl)-1H-pyrazole-4-carboxamide (Example E150);
N-(2-(trans-4-(5-isopropyl-1,3,4-oxadiazol-2-yl)cyclohexanecarboxamido)ethyl)-1-phenyl-3-(trifluoromethyl)-1H-pyrazole-4-carboxamide (Example E163);
N-(2-(trans-4-benzoylcyclohexanecarboxamido)ethyl)-1-(2-chlorophenyl)-3-(trifluoromethyl)-1H-pyrazole-4-carboxamide (Example E164);
4-(5-isopropyl-1,2,4-oxadiazol-3-yl)-N-(2-(1-phenyl-3-(trifluoromethyl)-1H-pyrazole-4-carboxamido)ethyl)piperidine-1-carboxamide (Example E225);
N-(2-(trans-4-(5-isopropyl-1,2,4-oxadiazol-3-yl)cyclohexanecarboxamido)ethyl)-1-phenyl-3-(trifluoromethyl)-1H-pyrazole-4-carboxamide (Example E228);
2-isopropyl-N-(2-(1-phenyl-3-(trifluoromethyl)-1H-pyrazole-4-carboxamido)ethyl)-4,5,6,7-tetrahydrobenzo[d]thiazole-6-carboxamide (Example E242);
1-(2-chlorophenyl)-N-(2-(trans-4-(5-isopropyl-1,2,4-oxadiazol-3-yl)cyclohexanecarboxamido)ethyl)-3-(trifluoromethyl)-1H-pyrazole-4-carboxamide (Example E257);
1-(2-fluorophenyl)-N-(2-(trans-4-(5-isopropyl-1,2,4-oxadiazol-3-yl)cyclohexanecarboxamido)ethyl)-3-(trifluoromethyl)-1H-pyrazole-4-carboxamide (Example E260);
1-(2-fluorophenyl)-N-(2-(trans-4-(5-isopropyl-1,3,4-oxadiazol-2-yl)cyclohexanecarboxamido)ethyl)-1H-indole-3-carboxamide (Example E265); or
trans-5-(5-isopropyl-1,3,4-oxadiazol-2-yl)-1-methyl-N-(2-(1-phenyl-3-(trifluoromethyl)-1H-pyrazole-4-carboxamido)ethyl)piperidine-2-carboxamide (Example E267);
   or a salt thereof.

As a salt of the compound of the present invention, a pharmacologically acceptable salt is preferable. Examples of such a salt include a salt with inorganic base, a salt with organic base, a salt with inorganic acid, a salt with organic acid, a salt with basic or acidic amino acid and the like.

Preferable examples of the salt with inorganic base include alkali metal salts such as sodium salt, potassium salt and the like; alkaline earth metal salts such as calcium salt, magnesium salt and the like; aluminum salt; ammonium salt and the like.

Preferable examples of the salt with organic base include a salt with trimethylamine, triethylamine, pyridine, picoline, ethanolamine, diethanolamine, triethanolamine, tromethamine[tris(hydroxymethyl)methylamine], tert-butylamine, cyclohexylamine, benzylamine, dicyclohexylamine, N,N-dibenzylethylenediamine or the like.

Preferable examples of the salt with inorganic acid include a salt with hydrochloric acid, hydrobromic acid, nitric acid, sulfuric acid, phosphoric acid or the like.

Preferable examples of the salt with organic acid include a salt with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid or the like.

Preferable examples of the salt with basic amino acid include a salt with arginine, lysine, ornithine or the like.

Preferable examples of the salt with acidic amino acid include a salt with aspartic acid, glutamic acid or the like.

A prodrug of the compound of the present invention is a compound that converts to the compound of the present invention due to the reaction by enzyme, gastric acid and the like under the physiological conditions in the body; that is, a compound that converts to the compound of the present invention by enzymatic oxidation, reduction, hydrolysis and the like, and a compound that converts to the compound of the present invention by hydrolysis and the like by gastric acid and the like. Examples of a prodrug of the compound of the present invention include a compound wherein an amino group of the compound of the present invention is acylated, alkylated or phosphorylated (e.g., a compound where amino group of the compound of the present invention is eicosanoylated, alanylated, pentylaminocarbonylated, (5-methyl-2-oxo-1,3-dioxolen-4-yl)methoxycarbonylated, tetrahydrofuranylated, pyrrolidyl methylated, pivaloyloxymethylated or tert-butylated); a compound wherein a hydroxy group of the compound of the present invention is acylated, alkylated, phosphorylated or borated (e.g., a compound where a hydroxy group of the compound of the present invention is acetylated, palmitoylated, propanoylated, pivaloylated, succinylated, fumarylated, alanylated or dimethylaminomethylcarbonylated); a compound wherein a carboxyl group of the compound of the present invention is esterified or amidated (e.g., a compound where a carboxyl group of the compound of the present invention is ethyl esterified, phenyl esterified, carboxymethyl esterified, dimethylaminomethyl esterified, pivaloyloxymethyl esterified, ethoxycarbonyloxyethyl esterified, phthalidyl esterified, (5-methyl-2-oxo-1,3-dioxolen-4-yl)methyl esterified, cyclohexyloxycarbonylethyl esterified or methylamidated) and the like. These compounds can be produced from the compound of the present invention according to a method known per se.

A prodrug of the compound of the present invention may be a compound that converts to the compound of the present invention under physiological conditions as described in Development of Pharmaceutical Products, vol. 7, Molecule Design, pp. 163-198, Hirokawa Shoten (1990).

The compound of the present invention may be labeled with an isotope (e.g., ³H, ¹⁴C, ³⁵S, ¹²⁵I and the like) and the like.

The compound of the present invention may be an anhydride or a hydrate.

The compound of the present invention and a prodrug thereof (hereinafter sometimes to be simply referred to as the compound of the present invention) show low toxicity and can be used as an agent for the prophylaxis or treatment of various diseases to be mentioned later for mammals (e.g., human, mouse, rat, rabbit, dog, cat, cattle, horse, swine, simian) as they are or by admixing with a pharmacologically acceptable carrier and the like to give a pharmaceutical composition.

Here, various organic or inorganic carriers conventionally used as materials for pharmaceutical preparations are used as a pharmacologically acceptable carrier, which are added as an excipient, a lubricant, a binder, a disintegrant and the like for solid preparations; and a solvent, a dissolution aid, a suspending agent, an isotonicity agent, a buffer, a soothing agent and the like for liquid preparations. Where necessary, an additive for pharmaceutical preparations such as a preservative, an antioxidant, a coloring agent, a sweetening agent and the like can be used.

Preferable examples of the excipient include lactose, sucrose, D-mannitol, D-sorbitol, starch, pregelatinized starch, dextrin, crystalline cellulose, low-substituted hydroxypropyl cellulose, sodium carboxymethylcellulose, powdered acacia, pullulan, light silicic anhydride, synthetic aluminum silicate, magnesium aluminate metasilicate and the like.

Preferable examples of the lubricant include magnesium stearate, calcium stearate, talc, colloidal silica and the like.

Preferable examples of the binder include pregelatinized starch, saccharose, gelatin, powdered acacia, methylcellulose, carboxymethylcellulose, sodium carboxymethylcellulose, crystalline cellulose, sucrose, D-mannitol, trehalose, dextrin, pullulan, hydroxypropyl cellulose, hydroxypropyl methylcellulose, polyvinylpyrrolidone and the like.

Preferable examples of the disintegrant include lactose, sucrose, starch, carboxymethylcellulose, calcium carboxymethylcellulose, sodium croscarmellose, sodium carboxymethyl starch, light silicic anhydride, low-substituted hydroxypropyl cellulose and the like.

Preferable examples of the solvent include water for injection, physiological brine, Ringer's solution, alcohol, propylene glycol, polyethylene glycol, sesame oil, corn oil, olive oil, cottonseed oil and the like.

Preferable examples of the dissolution aid include polyethylene glycol, propylene glycol, D-mannitol, trehalose, benzyl benzoate, ethanol, trisaminomethane, cholesterol, triethanolamine, sodium carbonate, sodium citrate, sodium salicylate, sodium acetate and the like.

Preferable examples of the suspending agent include surfactants such as stearyltriethanolamine, sodium lauryl sulfate, lauryl aminopropionate, lecithin, benzalkonium chloride, benzethonium chloride, glycerol monostearate and the like; hydrophilic polymers such as polyvinyl alcohol, polyvinylpyrrolidone, sodium carboxymethylcellulose, methylcellulose, hydroxymethylcellulose, hydroxyethylcellulose, hydroxypropyl cellulose and the like; polysorbates, polyoxyethylene hydrogenated castor oil, and the like.

Preferable examples of the isotonicity agent include sodium chloride, glycerol, D-mannitol, D-sorbitol, glucose and the like.

Preferable examples of the buffer include phosphate buffer, acetate buffer, carbonate buffer, citrate buffer and the like.

Preferable examples of the soothing agent include benzyl alcohol and the like.

Preferable examples of the preservative include p-oxybenzoates, chlorobutanol, benzyl alcohol, phenethyl alcohol, dehydroacetic acid, sorbic acid and the like.

Preferable examples of the antioxidant include sulfite, ascorbate and the like.

Preferable examples of the coloring agent include water-soluble edible tar pigments (e.g., food colors such as Food Color Red Nos. 2 and 3, Food Color Yellow Nos. 4 and 5, Food Color Blue Nos. 1 and 2 and the like), water insoluble lake pigments (e.g., aluminum salt of the aforementioned water-soluble edible tar pigment), natural pigments (e.g., beta carotene, chlorophil, red iron oxide) and the like.

Preferable examples of the sweetening agent include saccharin sodium, dipotassium glycyrrhizinate, aspartame, stevia and the like.

The dosage form of the aforementioned pharmaceutical composition is, for example, an oral agent such as tablets (inclusive of sublingual tablets and orally disintegrable tablets), capsules (inclusive of soft capsules and microcapsules), granules, powders, troches, syrups, emulsions, suspensions and the like; or a parenteral agent such as injections (e.g., subcutaneous injections, intravenous injections, intramuscular injections, intraperitoneal injections, drip infusions), external agents (e.g., transdermal preparations, ointments), suppositories (e.g., rectal suppositories, vaginal suppositories), pellets, nasal preparations, pulmonary preparations (inhalations), ophthalmic preparations and the like. These may be administered safely via an oral or parenteral route.

These agents may be controlled-release preparations such as rapid-release preparations and sustained-release preparations (e.g., sustained-release microcapsules).

The pharmaceutical composition can be produced according to a method conventionally used in the field of pharmaceutical preparation, such as the method described in Japan

Pharmacopoeia and the like. Concrete production methods of preparations are described in detail in the following.

While the content of the compound of the present invention in the pharmaceutical composition varies depending on the dosage form, dose of the compound of the present invention and the like, it is, for example, about 0.1-100 wt %.

Where necessary, the aforementioned oral agents may be coated with a coating base for the purpose of masking taste, enteric property or sustained release.

Examples of the coating base include a sugar-coating base, a water-soluble film coating base, an enteric film coating base, a sustained-release film coating base and the like.

As the sugar-coating base, sucrose may be used, if necessary, along with one or more species selected from talc, precipitated calcium carbonate, gelatin, powdered acacia, pullulan, carnauba wax and the like.

As the water-soluble film coating base, for example, cellulose polymers such as hydroxypropyl cellulose, hydroxypropyl methylcellulose, hydroxyethylcellulose, methylhydroxyethylcellulose and the like; synthetic polymers such as polyvinyl acetal diethylaminoacetate, aminoalkyl methacrylate copolymer E [Eudragit E, trade name, Roehm Pharma], polyvinylpyrrolidone and the like; polysaccharides such as pullulan and the like; and the like are used.

As the enteric film coating base, for example, cellulose polymers such as hydroxypropyl methylcellulose phthalate, hydroxypropyl methylcellulose acetate succinate, carboxymethylethylcellulose, cellulose acetate phthalate and the like; acrylic acid polymers such as methacrylic acid copolymer L [Eudragit L, trade name, Roehm Pharma], methacrylic acid copolymer LD [Eudragit L-30D55, trade name, Roehm Pharma], methacrylic acid copolymer S [Eudragit S, trade name, Roehm Pharma] and the like; natural products such as shellac and the like; and the like are used.

As the sustained-release film coating base, for example, cellulose polymers such as ethylcellulose and the like; acrylic acid polymers such as aminoalkyl methacrylate copolymer RS [Eudragit RS, trade name, Roehm Pharma], ethyl acrylate-methyl methacrylate copolymer suspension [Eudragit NE, trade name, Roehm Pharma] and the like; and the like are used.

Two or more kinds of the above-mentioned coating bases may be mixed in an appropriate ratio for use. In addition, a light shielding agent such as titanium oxide, ferric oxide and the like may be used during coating.

The compound of the present invention shows low toxicity (e.g., acute toxicity, chronic toxicity, genetic toxicity, reproductive to xicity, cardiotoxicity, carcinogenic), causes fewer side effects and can be used as an agent for the prophylaxis or treatment or diagnosis of various diseases for mammals (e.g., human, cattle, horse, dog, cat, simian, mouse, rat, especially human).

The compound of the present invention has a DGAT (DGAT1 or DGAT2 or both) inhibitory action, and is useful for the prophylaxis, treatment or amelioration of DGAT-related diseases.

As the DGAT-related diseases, for example, obesity, diabetes (e.g., type 1 diabetes, type 2 diabetes, gestational diabetes), insulin resistance, leptin resistance, arteriosclerosis, hyperlipidemia (e.g., hypertriglyceridemia, hypercholesterolemia, hypo-HDL-cholesterolemia, postprandial hyperlipemia), arteriosclerosis, hypertension, cardiac failure, metabolic syndrome and the like can be mentioned.

For diagnostic criteria of diabetes, Japan Diabetes Society reported new diagnostic criteria in 1999.

According to this report, diabetes is a condition showing any of a fasting blood glucose level (glucose concentration of intravenous plasma) of not less than 126 mg/dl, a 75 g oral glucose tolerance test (75 g OGTT) 2 h level (glucose concentration of intravenous plasma) of not less than 200 mg/dl, and a non-fasting blood glucose level (glucose concentration of intravenous plasma) of not less than 200 mg/dl. A condition not falling under the above-mentioned diabetes and different from “a condition showing a fasting blood glucose level (glucose concentration of intravenous plasma) of less than 110 mg/dl or a 75 g oral glucose tolerance test (75 g OGTT) 2 h level (glucose concentration of intravenous plasma) of less than 140 mg/dl” (normal type) is called a “borderline type”.

In addition, ADA (American Diabetes Association) reported new diagnostic criteria of diabetes in 1997 and WHO in 1998.

According to these reports, diabetes is a condition showing a fasting blood glucose level (glucose concentration of intravenous plasma) of not less than 126 mg/dl and a 75 g oral glucose tolerance test 2 h level (glucose concentration of intravenous plasma) of not less than 200 mg/dl.

According to the above-mentioned reports, impaired glucose tolerance is a condition showing a fasting blood glucose level (glucose concentration of intravenous plasma) of less than 126 mg/dl and a 75 g oral glucose tolerance test 2 h level (glucose concentration of intravenous plasma) of not less than 140 mg/dl and less than 200 mg/dl. According to the report of ADA, a condition showing a fasting blood glucose level (glucose concentration of intravenous plasma) of not less than 110 mg/dl and less than 126 mg/dl is called IFG (Impaired Fasting Glucose). According to the report of WHO, among the IFG (Impaired Fasting Glucose), a condition showing a 75 g oral glucose tolerance test 2 h level (glucose concentration of intravenous plasma) of less than 140 mg/dl is called IFG (Impaired Fasting Glycemia).

The compound of the present invention can be also used as an agent for the prophylaxis or treatment of diabetes, borderline type, impaired glucose tolerance, IFG (Impaired Fasting Glucose) and IFG (Impaired Fasting Glycemia), as determined according to the above-mentioned new diagnostic criteria. Moreover, the compound of the present invention can prevent progress of borderline type, impaired glucose tolerance, IFG (Impaired Fasting Glucose) or IFG (Impaired Fasting Glycemia) into diabetes.

The compound of the present invention can be also used as an agent for the prophylaxis or treatment of, for example, diabetic complications [e.g., neuropathy, nephropathy, retinopathy, cataract, macroangiopathy, osteopenia, hyperosmolar diabetic coma, infectious disease (e.g., respiratory infection, urinary tract infection, gastrointestinal infection, dermal soft tissue infection, inferior limb infection), diabetic gangrene, xerostomia, hypacusis, cerebrovascular disorder, peripheral blood circulation disorder], osteoporosis, cachexia (e.g., cancerous cachexia, tuberculous cachexia, diabetic cachexia, blood disease cachexia, endocrine disease cachexia, infectious disease cachexia or cachexia due to acquired immunodeficiency syndrome), fatty liver, polycystic ovary syndrome, kidney disease (e.g., diabetic nephropathy, glomerular nephritis, glomerulosclerosis, nephrotic syndrome, hypertensive nephrosclerosis, end stage kidney disease), muscular dystrophy, myocardial infarction, angina pectoris, cerebrovascular accident (e.g., cerebral infarction, cerebral apoplexy), Alzheimer's disease, Parkinson's syndrome, anxiety, dementia, insulin resistance syndrome, Syndrome X, hyperinsulinemia, hyperinsulinemia-induced sensory disorder, tumor (e.g., leukemia, breast cancer, prostatic cancer, skin cancer), irritable bowel syndrome, acute or chronic diarrhea, inflammatory diseases (e.g., chronic rheumatoid arthritis, spondylitis deformans, osteoarthritis, lumbago, gout, postoperative or traumatic inflammation, tumentia, neuralgia, pharyngolaryngitis, cystitis, hepatitis (inclusive of nonalcoholic steatohepatitis), pneumonia, pancreatitis, enteritis, inflammatory bowel diseases (including inflammatory disease of large intestine), ulcerative colitis, gastric mucosal injury (inclusive of gastric mucosal injury caused by aspirin)), small intestine mucous membrane trauma, malabsorption, testis function disorder, visceral obesity syndrome and the like.

The compound of the present invention can also be used for the secondary prophylaxis or suppression of the progression of the above-mentioned various diseases (e.g., cardiovascular events such as cardiac infarction and the like).

While the dose of the compound of the present invention varies depending on the administration subject, administration route, target disease, condition and the like, the compound of the present invention is generally given in a single dose of about 0.01-100 mg/kg body weight, preferably 0.05-30 mg/kg body weight, more preferably 0.1-10 mg/kg body weight, in the case of, for example, oral administration to adult diabetic patients. This dose is desirably given 1 to 3 times a day.

The compound of the present invention can be used in combination with drugs such as a therapeutic agent for diabetes, a therapeutic agent for diabetic complications, an antihyperlipemic agent, an antihypertensive agent, an antiobestic agent, a diuretic, an antithrombotic agent and the like (hereinafter to be referred to as a combination drug), with the aim of enhancing the action of the compound, reducing the dose of the compound and the like. In this case, the timing of administration of the compound of the present invention and a combination drug is not limited. These may be simultaneously administered to an administration subject or administered in a staggered manner. Moreover, the compound of the present invention and a combination drug may be administered as two kinds of preparations each containing an active ingredient, or may be administered as a single preparation containing both active ingredients.

The dose of the combination drug can be determined as appropriate based on the dose clinically employed. The proportion of the compound of the present invention and the combination drug can be appropriately determined depending on the administration subject, administration route, target disease, condition, combination and the like. When, for example, the administration subject is human, the combination drug is used in an amount of 0.01-100 parts by weight per 1 part by weight of the compound of the present invention.

As the therapeutic agent for diabetes, insulin preparations (e.g., animal insulin preparations extracted from the pancreas of bovine or pig; human insulin preparations genetically synthesized using Escherichia coli or yeast; zinc insulin; protamine zinc insulin; fragment or derivative of insulin (e.g., INS-1), oral insulin preparation), insulin sensitizers (e.g., pioglitazone or a salt thereof (preferably hydrochloride), rosiglitazone or a salt thereof (preferably maleate), Reglixane (JTT-501), Netoglitazone (MCC-555), DRF-2593, KRP-297, R-119702, Rivoglitazone (CS-011), FK-614, compounds described in WO99/58510 (e.g., (E)-4-[4-(5-methyl-2-phenyl-4-oxazolylmethoxy)benzyloxyimino]-4-phenylbutyric acid), compounds described in WO01/38325, Tesaglitazar (AZ-242), Ragaglitazar (N,N-622), Muraglitazar (BMS-298585), ONO-5816, Edaglitazone (BM-13-1258), LM-4156, MBX-102, Naveglitazar (LY-519818), MX-6054, LY-510929, Balaglitazone (N,N-2344), T-131 or a salt thereof, THR-0921), PPARγ agonists, PPARγ antagonists, PPARγ/α, dual agonists, α-glucosidase inhibitors (e.g., voglibose, acarbose, miglitol, emiglitate), biguanides (e.g., phenformin, metformin, buformin or salts thereof (e.g., hydrochloride, fumarate, succinate)), insulin secretagogues [sulfonylureas (e.g., tolbutamide, glibenclamide, gliclazide, chlorpropamide, tolazamide, acetohexamide, glyclopyramide, glimepiride, glipizide, glybuzole), repaglinide, senaglinide, nateglide, mitiglinide or calcium salt hydrate thereof], GPR40 agonists, GLP-1 receptor agonists [e.g., GLP-1, GLP-1MR, N,N-2211, AC-2993 (exendin-4), BIM-51077, Aib(8.35) hGLP-1 (7.37)NH₂, CJC-1131], amylin agonists (e.g., pramlintide), phosphotyrosine phosphatase inhibitors (e.g., sodium vanadate), dipeptidyl peptidase IV inhibitors (e.g., NVP-DPP-728, PT-100, P32/98, Vidagliptin (LAF-237), P93/01, TS-021, Sitagliptin (MK-431), Saxagliptin (BMS-477118), T-6666), β3 agonists (e.g., AJ-9677, AZ40140), gluconeogenesis inhibitors (e.g., glycogen phosphorylase inhibitors, glucose-6-phosphatase inhibitors, glucagon antagonists), SGLT (sodium-glucose cotransporter) inhibitors (e.g., T-1095), 11β-hydroxysteroid dehydrogenase inhibitors (e.g., BVT-3498), adiponectin or agonists thereof, IKK inhibitors (e.g., AS-2868), leptin resistance improving drugs, somatostatin receptor agonists (compounds described in WO01/25228, WO03/42204, WO98/44921, WO98/45285 and WO99/22735) and glucokinase activators (e.g., Ro-28-1675) can be mentioned.

Examples of the therapeutic agent for diabetic complications include aldose reductase inhibitors (e.g., Tolrestat, Epalrestat, Zenarestat, Zopolrestat, Minalrestat, Fidarestat, CT-112, Ranirestat), neurotrophic factors and increasing drugs thereof (e.g., NGF, NT-3, BDNF, neurotrophin production-secretion promoters described in WO01/14372 (e.g., 4-(4-chlorophenyl)-2-(2-methyl-1-imidazolyl)-5-[3-(2-methylphenoxy)propyl]oxazole)), neuranagenesis stimulators (e.g., Y-128), PKC inhibitors (e.g., ruboxistaurin mesylate), AGE inhibitors (e.g., ALT946, pimagedine, pyratoxanthine, N-phenacyithiazolium bromide (ALT766), ALT-711, EXO-226, Pyridorin, Pyridoxamine), reactive oxygen scavengers (e.g., thioctic acid), cerebral vasodilators (e.g., tiapride, mexiletine), somatostatin receptor agonists (e.g., BIM23190) and apoptosis signal regulating kinase-1 (ASK-1) inhibitors.

Examples of the antihyperlipemic agent include HMG-CoA reductase inhibitors (e.g., pravastatin, simvastatin, lovastatin, atorvastatin, fluvastatin, itavastatin, rosuvastatin, pitavastatin and salts thereof (e.g., sodium salt, calcium salt)), squalene synthase inhibitors (e.g., compounds described in WO97/10224, such as N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]-piperidine-4-acetic acid), fibrate compounds (e.g., bezafibrate, clofibrate, simfibrate, clinofibrate), ACAT inhibitors (e.g., Avasimibe, Eflucimibe), anion exchange resins (e.g., colestyramine), probucol, nicotinic acid drugs (e.g., nicomol, niceritrol), ethyl icosapentate and plant sterols (e.g., soysterol, γ-oryzanol).

Examples of the antihypertensive agent include angiotensin converting enzyme inhibitors (e.g., captopril, enalapril, delapril), angiotensin II receptor antagonists (e.g., candesartan cilexetil, losartan, eprosartan, valsartan, telmisartan, irbesartan, tasosartan, 1-[[2′-(2,5-dihydro-5-oxo-4H-1,2,4-oxadiazol-3-yl)biphenyl-4-yl]methyl]-2-ethoxy-1H-benzimidazole-7-carboxylic acid), calcium antagonists (e.g., manidipine, nifedipine, amlodipine, efonidipine, nicardipine), potassium channel openers (e.g., levcromakalim, L-27152, AL 0671, NIP-121) and Clonidine.

Examples of the antiobestic agent include antiobestic agents acting on the central nervous system (e.g., Dexfenfluramine, fenfluramine, phentermine, Sibutramine, amfepramone, dexamphetamine, Mazindol, phenylpropanolamine, clobenzorex; MCH receptor antagonists (e.g., SB-568849; SNAP-7941; compounds encompassed in WO01/82925 and WO01/87834); neuropeptide Y antagonists (e.g., CP-422935); cannabinoid receptor antagonists (e.g., SR-141716, SR-147778); ghrelin antagonists; 11β-hydroxysteroid dehydrogenase inhibitors (e.g., BVT-3498)), pancreatic lipase inhibitors (e.g., orlistat, ATL-962), β3 agonists (e.g., AJ-9677, AZ40140), peptidic anorexiants (e.g., leptin, CNTF (Ciliary Neurotropic Factor)), cholecystokinin agonists (e.g., lintitript, FPL-15849) and feeding deterrents (e.g., P-57).

Examples of the diuretic include xanthine derivatives (e.g., sodium salicylate and theobromine, calcium salicylate and theobromine), thiazide preparations (e.g., ethiazide, cyclopenthiazide, trichloromethyazide, hydrochlorothiazide, hydroflumethiazide, benzylhydrochlorothiazide, penflutizide, polythiazide, methyclothiazide), antialdosterone preparations (e.g., spironolactone, triamterene), carbonate dehydratase inhibitors (e.g., acetazolamide), chlorobenzenesulfonamide preparations (e.g., chlortalidone, mefruside, indapamide), azosemide, isosorbide, etacrynic acid, piretanide, bumetanide and furosemide.

Examples of the antithrombotic agent include heparins (e.g., heparin sodium, heparin calcium, dalteparin sodium), warfarins (e.g., warfarin potassium), anti-thrombin drugs (e.g., aragatroban), thrombolytic agents (e.g., urokinase, tisokinase, alteplase, nateplase, monteplase, pamiteplase), platelet aggregation inhibitors (e.g., ticlopidine hydrochloride, cilostazol, ethyl icosapentate, beraprost sodium, sarpogrelate hydrochloride) and the like.

Hereinafter the production methods of the compound of the present invention are explained.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and diastereomers, and mixtures, racemate or otherwise, thereof. Accordingly, this invention also includes all such isomers, including diastereomeric mixtures, enantiomeric mixtures, pure diastereomers and pure enantiomers of the compounds of this invention. The term “enantiomer” refers to two stereoisomers of a compound which are non-superimposable mirror images of one another. The term “diastereomer” refers to a pair of optical isomers which are not mirror images of one another. Diastereomers have different physical properties, e.g. melting points, boiling points, spectral properties, and reactivities. The compounds of the present invention may also exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

In the structures shown herein, where the stereochemistry of any particular chiral atom is not specified, then all stereoisomers are contemplated and included as the compounds of the invention. Where stereochemistry is specified by a solid wedge or a hashed wedge representing a particular configuration, then that stereoisomer is so specified and defined. When stereochemistry is specified by a solid line or a hashed line representing a relative conformation such as cis and trans, then that conformation is so specified and defined.

In the present specification, the following abbreviations may be used:

### DCM: Dichloromethane

### MeCN: Acetonitrile

### THF: Tetrahydrofuran

### EtOH: Ethanol

### MeOH: Methanol

iPrOH: Isopropanol

### CHCl₃: Chloroform

### DCE: Dichloroethane

DMSO: Dimethyl sulfoxide

### DMF: Dimethylformamide

### DMA: Dimethylacetamide

AcOEt: Ethyl acetate
Et₂O: Diethyl ether

### CH₃CN: Acetonitrile

### H₂O: Water

HATU: O-(7-Azabenzotriazol-1-yl)-N,N,N′,N′-tetramethyluronium hexafluorophosphate
BOP—Cl: Benzotriazol-1-yl-oxytris(dimethylamino)phosphonium hexafluorophosphate
EDAC.HCl: 1-Ethyl-3-(3-dimethylaminopropyl)carbodiimide hydrochloride
HOBt.H₂O: Hydroxybenzotriazole monohydrate
NaOH: Sodium hydroxide
KOH: Potassium hydroxide
K₂CO₃: Potassium carbonate
Cs₂CO₃: Cesium carbonate
Na₂CO₃: Sodium carbonate

### TEA: Triethylamine

DIPEA: Diisopropylethyl amine

### Py: Pyridine

NaH: Sodium hydride
LDA: Lithium diisopropylamide
NaHCO₃: Sodium hydrogencarbonate
MP-carbonate: Macroporous triethylammonium methylpolystyrene carbonate
PS-carbodiimide: N-Cyclohexylcarbodiimide-N′-propyloxymethyl polystyrene
PS-trisamine: Tris-(2-aminoethyl)amine polystyrene
PS-isocyanate: Polystyrene methylisocyanate

### TFP: Tetrafluorophenol

H₂SO₄: Sulfuric acid
HCl: Hydrochloric acid
HBr: Hydrobromic acid
NH₄Cl: Ammonium chloride
TFA: Trifluoroacetic acid
AcOH: Acetic acid
TFAA: Trifluoroacetic anhydride
Na₂SO₄: Sodium sulfate
MgSO₄: Magnesium sulfate
TsCl: p-Toluenesulfonyl chloride
MCPBA: m-Chloroperbenzoic acid

### CDI: N,N-Carbonyldiimidazole

PtO₂: Platinum oxide
Pd/C: Palladium on carbon
DMF-DMA: Dimethylformamide dimethylacetal
POCl₃: Phosphorus oxychloride
TFFH: Tetramethylfluoroformamidinium hexafluorophosphate
Boc: tert-Butoxycarbonyl

### Cbz: Benzyloxycarbonyl

For illustrative purposes, Schemes 1-24 show general methods for preparing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### General Synthetic Methods for Compound (Ie)

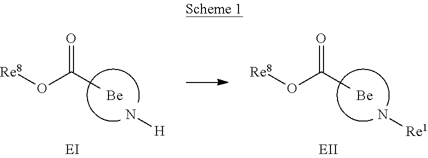

wherein Re⁸ is a C₁-C₄ alkyl or benzyl group and other symbols are as defined above.

Intermediate esters EII, which are suitable for use in preparing compounds of formulas Ie, Ie-I, Ie-IV and as shown in Schemes 2, 3, 5 and 6, can be prepared under various conditions depending on the nature of the Re¹ substituent. In the case wherein Re¹ is an optionally substituted aryl or heteroaryl group, esters EII can be prepared according to one of the following references: Tetrahedron Lett. 1998, 39, 2941-2944; Eur. J. Org. Chem. 2004, 695-709; J. Am. Chem. Soc 2001, 123, 7727-7729; J. Am. Chem. Soc. 2002, 124, 11684-11688; J. Org. Chem. 2004, 69, 5578. Typically, the N-Arylation of the Be ring is performed with the corresponding aryl halide (preferably iodide) (including a heteroaryl halide) in the presence of copper catalyst such as copper iodide or copper oxide, in the presence of a ligand such as substituted ethylene diamines, salicylaldoximes or other ligands reported in Eur. J. Org. Chem. 2004, 695-709. The reaction requires a base such as potassium phosphate or cesium carbonate and is performed in a degassed solvent such as acetonitrile, toluene or DMF at a temperature of 20° C. to 150° C. for 0.5 to 48 hours under inert atmosphere. Preferably, the N-arylation is conducted according to the method described in J. Org. Chem. 2004, 69, 5578, in toluene with 1 equivalent of EI, 1.1-10 equivalents of aryl halide, 2 equivalents of diamine ligand, 2-3 equivalents of base and 0.05 equivalents of copper(I) iodide or according to the method described in Eur. J. Org. Chem. 2004, 695-709, in DMF with 1 equivalent of EI, 1.5-10 equivalents of aryl halide, 0.2-0.4 equivalents of oxime ligand, 2-3 equivalents of base and 0.05 equivalents of copper(II) oxide.

In the case where Re¹ is an alkyl or cycloalkyl group, esters EII can be prepared by direct alkylation with the corresponding alkyl halide (including a cycloalkyl halide) or the corresponding alkyl sulfonate (including a cycloalkyl sulfonate) in the presence of a base such as potassium carbonate, cesium carbonate or sodium hydride in a solvent such as DMF at a temperature ranging from 20° C. to 130° C. for 0.5 to 48 hours. In the case of hindered or poorly reactive alkyl halide, the alkyl halide may be used as the solvent at a temperature ranging from 20° C. to 130° C. for 0.5 to 48 hours. Alternatively, esters EII can be prepared from the amines EI by opening of the corresponding epoxide in the presence of a base such as potassium carbonate or cesium carbonate in a solvent such as halogenated hydrocarbons or neat at a temperature from 20° C. to 100° C. for 1 to 48 hours. Preferably, the alkylation is run in DMF or halogenated hydrocarbons with 1 equivalent of EI, 1.1-10 equivalents of alkyl halide, alkyl sulfonate or epoxide and 1-5 equivalents of base.

In the case where Re¹ is an acyl group, esters EII can be prepared with the corresponding acid halides or sulfonyl halides in the presence of a base such as sodium hydride, potassium carbonate, sodium hydroxide or triethylamine in a solvent such as DMF, acetone or halogenated hydrocarbons at a temperature ranging from 0° C. to 130° C. for 0.5 to 24 hours. Preferably, this reaction is run in DMF or halogenated hydrocarbons with 1 equivalent of EI, 1.1-2 equivalents of sulfonyl or acyl halide and 1-5 equivalents of base.

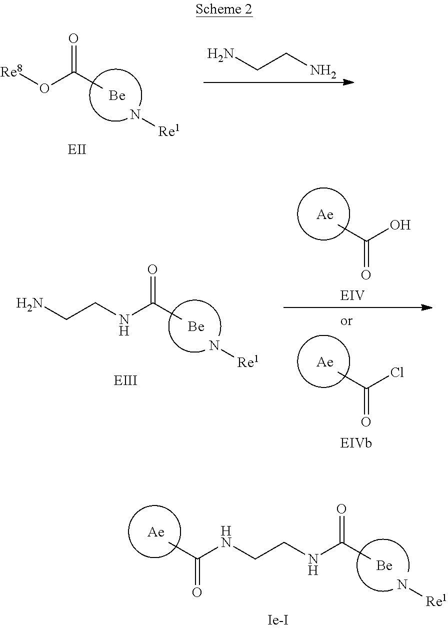

wherein the symbols are as defined above.

Compounds Ie-I can be prepared according to the sequence described in Scheme 2. Esters EII (Re⁸ is preferably methyl or ethyl group) can be treated with ethylenediamine at refluxing temperature to produce amines EIII. Compounds Ie-I can be conveniently prepared from an amine EIII or its salt and an acid ETV in the presence of various condensing reagents. Known reagents that effect amide bond formation include N,N-carbonyldiimidazole, halopyridine salts, 2,4,6-trichlorobenzoyl chloride, HATU, BOP—Cl or EDAC.HCl/HOBt.H₂O. In the present invention, the preferred reagent is EDAC.HCl/HOBt.H₂O. The reaction can be conducted in a variety of aprotic solvents such as halogenated hydrocarbons, acetonitrile or dimethylformamide, or a mixture of these solvents, at a temperature from 0° C. to 130° C., preferably 20° C. to 70° C. for a time ranging from 0.5 to 48 hours. A base such as triethylamine or diisopropylethylamine may be used especially if the reacting amine is in a salt form. While the amount of reagent varies depending on the condensation reagent used, the following stoichiometry is used preferably with EDAC.HCl/HOBt.H₂O: amine or its salt (1 equivalent), acid (1 equivalent), EDAC.HCl (1 to 2 equivalents), HOBt.H₂O (1 to 2 equivalents) and base (1 to 3 equivalents). Compounds Ie-I can also be prepared from an acid chloride EIVb and an amine EIII in the presence of a base such as triethylamine, diisopropylethylamine or pyridine in an aprotic solvent such as THF, benzene or halogenated hydrocarbons at temperatures from 20° C. to 90° C. for 0.5 to 24 hours.

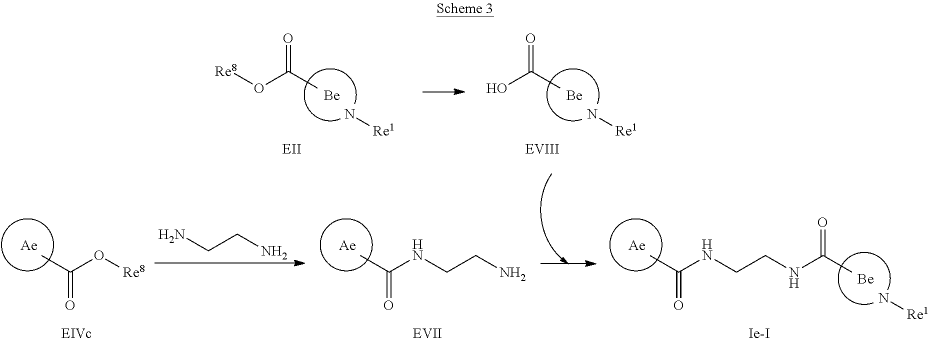

wherein the symbols are as defined above.

Alternatively, compounds Ie-I can be prepared following the sequence described in Scheme 3. Esters EIVc (Re⁸ is preferably methyl or ethyl group) can be treated with ethylenediamine at refluxing temperature to produce amines EVII. Compounds Ie-I can be prepared in the conditions mentioned above (Scheme 2) from the amine EVII or its salt and acid EVIII. Acids EVIII can be prepared from the corresponding esters EII (Scheme 1) by using a base such as lithium hydroxide, sodium hydroxide, carbonates (potassium or cesium) in a polar protic solvent such as methanol, ethanol, water or in mixtures of solvents including alcohols and water, or aprotic solvents. Typically, the hydrolysis is performed in an alcohol (methanol or ethanol) or in a 1:1 mixture of alcohol/THF, with water in the presence of sodium hydroxide (1-10 equivalents) at a temperature ranging from 20° C. to 100° C. for 0.5 to 24 hours. Acids EVIII can also be prepared from the corresponding esters EII by acid hydrolysis using an acid such as TFA, HCl, H₂SO₄, AcOH or in a mixture of these acids in neat or aqueous condition at a temperature ranging from 20° C. to 100° C. for 0.5 to 24 hours. In the case wherein Re⁸ is benzyl group, acids EVIII can be prepared from the corresponding esters EII by hydrogenolysis using catalysts such as palladium on carbon or palladium hydroxide in a protic solvent such as EtOH or aprotic solvent such as EtOAc under hydrogen atmosphere at a pressure of 15 to 150 psi, at a temperature of 20° C. to 100° C. for 1 to 48 hours. Additional conditions for the hydrolysis of ester groups can be found in T. W. Green, Protective Groups in Organic Synthesis, John Wiley and Sons, Inc., 1981.

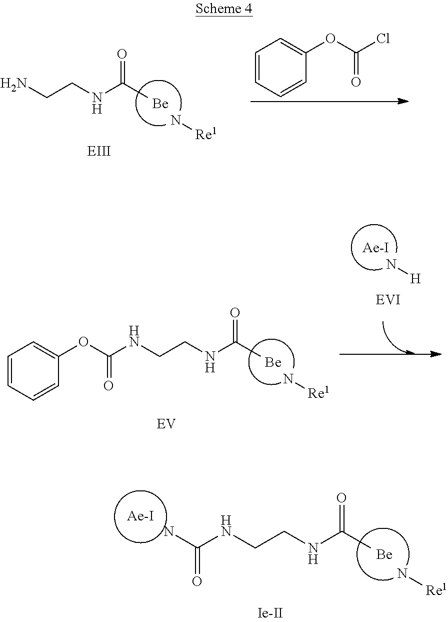

wherein Ae-I is an optionally substituted nitrogen-containing non-aromatic ring and other symbols are as defined above.

Compounds Ie-II can be prepared according to the sequence described in Scheme 4. Compounds EV can be prepared by reacting an amine EIII with a chloroformate in an aprotic vent such as halogenated hydrocarbons in the presence of a base such as triethylamine or pyridine at a temperature from 0° C. to 50° C. for 1 to 24 hours. In the present invention, the chloroformate of choice is phenyl chloroformate (1-2 equivalents) and the reaction is preferably run in dichloromethane at 20° C. for 2-3 hours in the presence of triethylamine (1.5-3 equivalents). Compounds Ie-II are conveniently prepared by reacting a carbamate EV with an amine EVI or its salt. The reaction is conducted neat or in a polar protic solvent such as alcohols at a temperature ranging from 120° C. to 170° C. for 15 minutes to 5 hours. A base such as potassium carbonate, cesium carbonate, pyridine, triethylamine or diisopropylethylamine may be used. When the amine EVI is used as a salt, the reaction is conducted in a polar protic solvent preferably an alcohol at a temperature ranging from 80° C. to 160° C. for 15 minutes to 5 hours in the presence of a base such as potassium carbonate, cesium carbonate, triethylamine, diisopropylethylamine. In the present invention, when the amine EVI is not a salt, the reaction is preferably run neat or in ethanol with equimolar ratio of amine EVI and carbamate EV. When amine EVI is a salt, the reaction is preferably run in refluxing ethanol with 1 equivalent of carbamate EV, 1-3 equivalents of amine EVI and 1-5 equivalents of base.

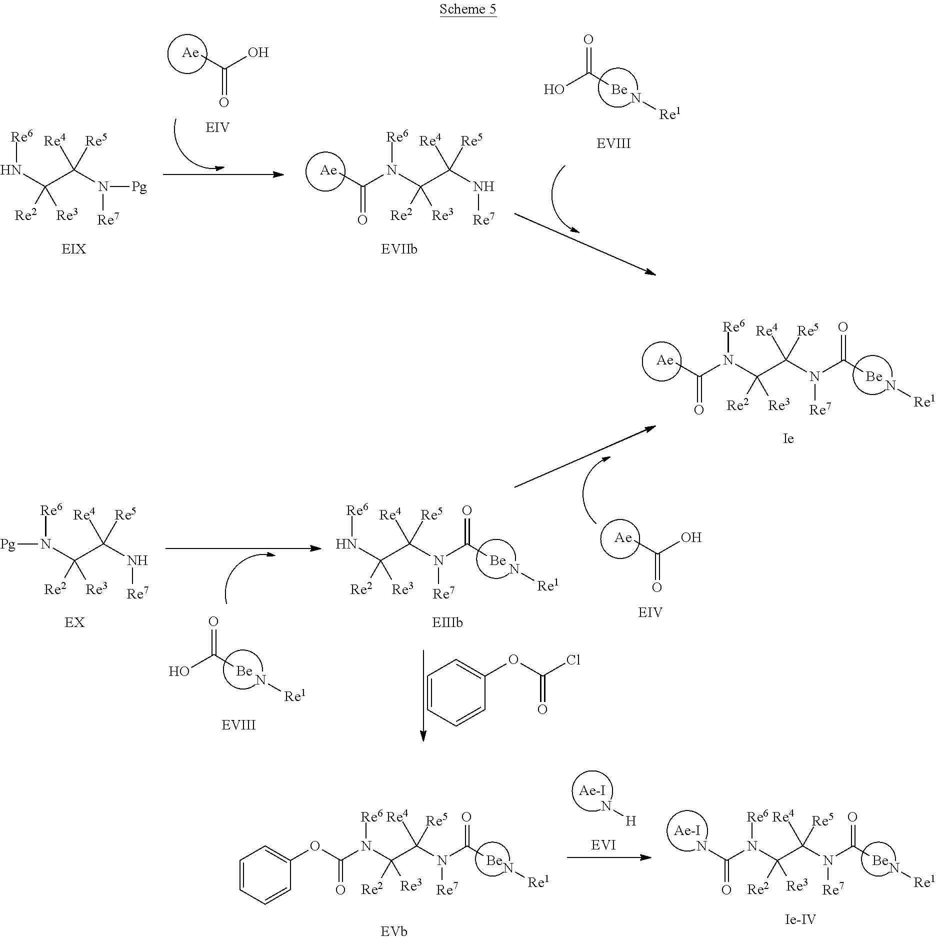

wherein Pg is a protecting group and other symbols are as defined above.

Compounds Ie and Ie-IV can be prepared according to Scheme 5. Compounds EVIIb can be the result of an amide coupling between a suitably protected amine EIX (Pg is preferably Boc or Cbz group) and an acid EIV in conditions commonly employed to form amide bonds followed by deprotection of the amino group. In the preferred case wherein Pg is Boc group, the deprotection is conveniently performed in the presence of acids such as TFA or HCl, neat or in a solvent such as ethyl ether or dioxane at a temperature from 0° C. to 100° C. for 5 minutes to 24 hours. Additional conditions for the deprotection of amines can be found in T. W. Green, Protective Groups in Organic Synthesis, John Wiley and Sons, Inc., 1981. In the present invention, the preferred deprotection method for Boc-protected amines consists in treating the protected amine in TFA or in 4N HCl in dioxanes at 20° C. for 10 minutes to 24 hour. Compounds EVIIb can be further coupled to an acid EVIII in conditions commonly employed to form amide bonds to afford compounds Ie.

Alternatively, compounds EIIIb can be the result of the coupling between a suitably protected amine EX (Pg is preferably Boc or Cbz group) and an acid EVIII under conditions commonly employed to form amide bonds followed by deprotection of the amino group. Compounds Ie can be produced by further coupling the amine EIIIb with an acid EIV under conditions commonly employed to form amide bonds.

Compounds Ie-IV can be prepared according to Scheme 5. Compounds EVb can be the result of the reaction of the previously described amine EIIIb with phenyl chloroformate following conditions described in Scheme 4. Compound Ie-IV is conveniently prepared by reacting a carbamate EVb with an amine EVI or its salt under conditions described in Scheme 4.

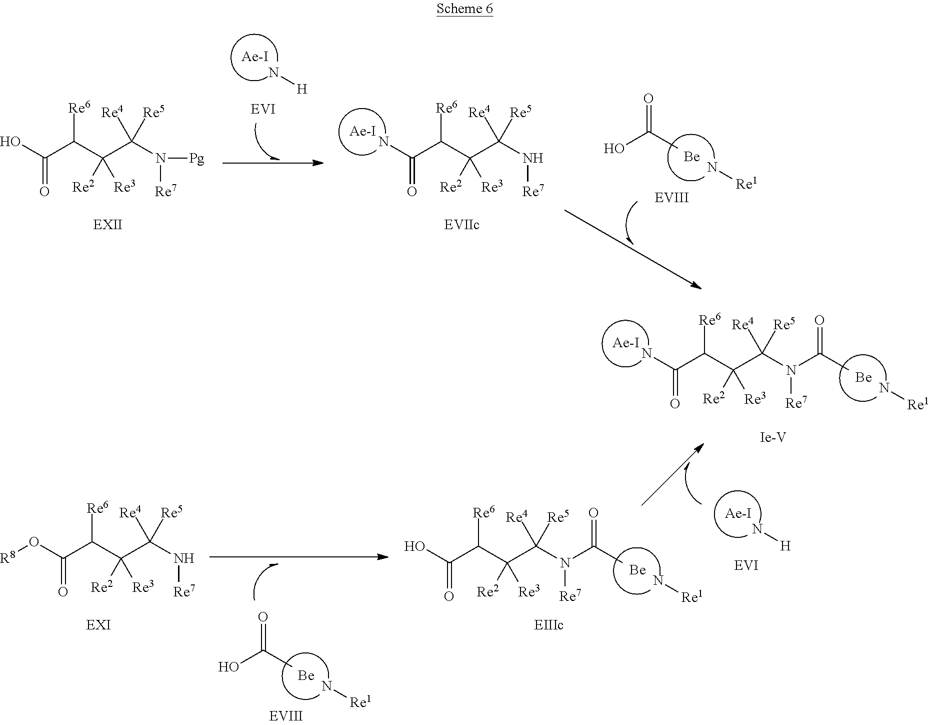

wherein the symbols are as defined above.

Compounds Ie-V can be prepared according to Scheme 6. Compounds EVIIc can be obtained by coupling a suitably N-protected (preferably Boc group) amino-acid EXII with an amine EVI or its salt under conditions commonly employed to form amide bonds, followed by removal of the Pg group according to known methods. Compounds Ie-V are then the result of the coupling between an acid EVIII and the amine EVIIc under conditions commonly employed to form amide bonds.

Alternatively, compounds EIIIc can be obtained by coupling an amino acid ester EXI (Re⁸ is preferably methyl or ethyl group) with an acid EVIII under conditions commonly employed to form amide bonds followed by hydrolysis of the ester according to known methods. Compounds Ie-V are then the result of the coupling between an acid EIIIc and an amine EVI or its salt under conditions commonly employed to form amide bonds.

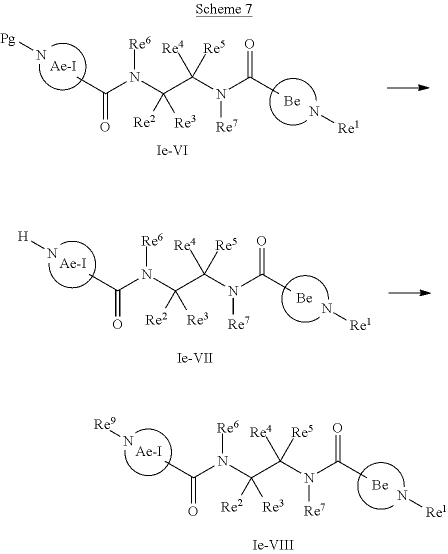

wherein Re⁹ is a substituent and other symbols are as defined above.

As the “substituent” for Re⁹, those exemplarily recited as “substituent” for Re¹, Re², Re³, Re⁴, Re⁵, Re⁶ or Re⁷, can be mentioned.

Methods for preparing compounds Ie-VII and Ie-VIII are presented in Scheme 7. Compounds Ie-VII can be prepared from Ie-VI, which may be obtained by similar methods to those described in Schemes 5, by deprotecting the amino group according to known methods. Compounds Ie-VIII wherein Re⁹ is an alkyl group can be prepared by treating an amine Ie-VII or its salt with the corresponding alkyl halide or alkyl sulfonate in the presence of an organic base such as pyridine, triethylamine, diisopropylethylamine or an inorganic base such as potassium carbonate or cesium carbonate. Solvents include halogenated hydrocarbons, THF or DMF. The transformation can also be accomplished by reductive alkylation by treatment with the corresponding aldehyde or ketone in the presence of a reducing agent such as sodium borohydride, sodium cyanoborohydride or sodium triacetoxyborohydride in solvents such as halogenated hydrocarbons. An acid such as acetic acid may be added to the reaction. In the present invention, the amine Ie-VII is preferably reacted with the corresponding aldehyde or ketone (1.1-2 equivalents), and the obtained imine is reduced in the presence of a reducing agent (1.5-3 equivalents) at low pH. Compounds Ie-VIII wherein Re⁹ is an acyl group can be prepared by treating an amine Ie-VII or its salt with the corresponding acid in the presence of a coupling agent under conditions commonly employed to form amide bonds. In this invention, the coupling agent of choice is EDAC.HCl/HOBt.H₂O in a solvent such as halogenated hydrocarbons or DMF at room temperature. The transformation may also be accomplished by treating an amine Ie-VII or its salt with the corresponding acid halide, acid anhydride, sulfonyl halide, isocyanate, carbamic halide, haloformate or dicarbonate in the presence of a base such as potassium carbonate, pyridine, triethylamine or diisopropylamine in a solvent such as acetone, THF, halogenated hydrocarbons or DMF at a temperature from 20° C. to 130° C. for 1 to 72 hours. Compounds Ie-VIII wherein Re⁹ is an aryl or heteroaryl group can be prepared by reacting an amine Ie-VII or its salt with the corresponding activated aryl halide (including a heteroaryl halide) under S_{N}Ar conditions (basic conditions in a polar, protic solvent; suitable bases include potassium hydride, sodium hydride, potassium tert-butoxide, lithium hydroxide or cesium/potassium carbonate in solvents such as DMF, DMSO or THF) or the corresponding aryl halide under palladium mediated conditions (conditions for these transformations can be found in Angew. Chem. Int. Ed., 1998, 37, 2046 or Organomet. Chem. 1999, 576, 125).

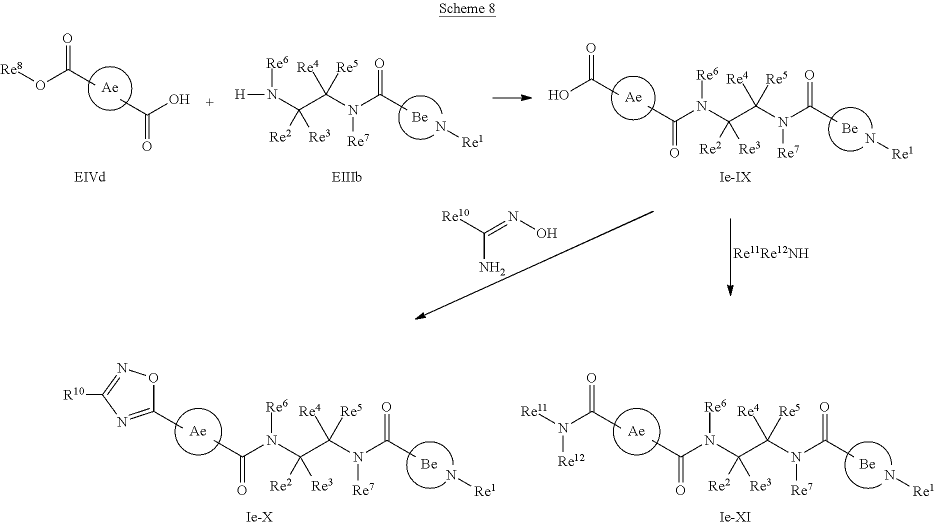

wherein Re¹⁰ and Re¹¹ are each independently an optionally substituted hydrocarbon group or an optionally substituted heterocyclic group, Re¹² is a hydrogen atom, an optionally substituted hydrocarbon group or an optionally substituted heterocyclic group, or is optionally bonded to Re¹¹ to form a non-aromatic ring, and other symbols are as defined above.

As the “optionally substituted hydrocarbon group” and “optionally substituted heterocyclic group” for Re¹⁰, Re¹¹ or Re¹²/those exemplarily recited as the “optionally substituted hydrocarbon group” and “optionally substituted heterocyclic group”, which are those exemplarily recited as the “substituent” for Re¹, Re², Re⁵, Re⁴, Re⁵, Re⁶ or Re⁷, can be mentioned.

As the “non-aromatic ring” formed by Re^{1l} and Re¹² bonded to each other, a non-aromatic heterocycle, which is exemplarily recited as the “substituent” for Re¹, Re², Re³, Re⁴, Re⁵, Re⁶ or Re⁷, can be mentioned.

The synthesis of compounds Ie-IX, Ie-X and Ie-XI is presented in Scheme 8. Compounds Ie-IX may be prepared by reacting a carboxylic acid EIVd with an amine EIIIb or its salt under conditions commonly employed to form amide bonds, followed by hydrolysis of the ester group. Acids Ie-IX can be transformed to amides Ie-XI by reaction with an amine or its salt (Re¹¹Re¹²NH) under conditions commonly employed to form amide bonds. When large numbers of amides Ie-XI are to be produced, acids Ie-IX and amines or their salt (Re¹¹Re¹²NH) may be treated with polymer supported coupling reagents such as polystyrene supported CDI (PS-CDI) (2-3 equivalents) in solvents such as DMF or THF or a mixture of solvents at a temperature from 20° C. to 80° C. for 1 to 72 hours. In the later conditions, HOBt.H₂O (0.1-0.5 equivalents) may be added and the excess amine and acid may be scavenged from the reaction by addition of polymer supported trisamine (0.5-1 equivalent) and isocyanate (0.5-1 equivalent). A polymer supported base such as polystyrene carbonate (PS-carbonate) (0.1-1 equivalent) may also be added when the amine is used as a salt.

Compounds Ie-X wherein the ring Ae is linked to a 3-substituted-1,2,4-oxadiazole can be prepared by treating an acid Ie-IX, or the corresponding acid chloride, with a substituted hydroxyamidine or its salt in the presence of a reagent such as a base, CDI, DCC/benzotriazole, DCC or TFFH (Synthesis 2004, (15), 2485-2492) in solvents such as DMF, THF, ACN or halogenated hydrocarbons. In the present invention, the acid Ie-IX is preferably treated with TFFH (1-1.5 equivalents) in the presence of a base such as triethylamine or diisopropylethylamine (1-1.5 equivalents) in a solvent such as DMF for 1 to 3 hours then treated with a substituted hydroxyamidine (1-1.5 equivalents) or its salt in a solvent such as DMF for 1 to 24 hours at a temperature of 100° C. according to the procedure described in J. Med. Chem. 2004, 12, 3111.

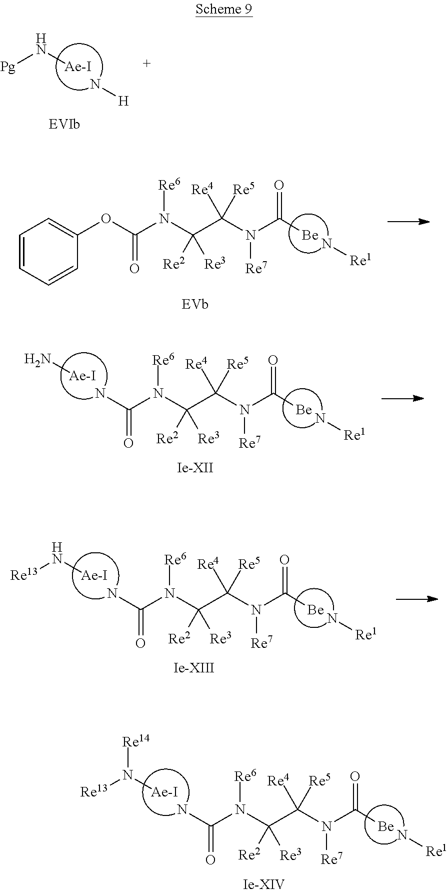

wherein Re¹³ and Re¹⁴ are each independently an optionally substituted hydrocarbon group, an optionally substituted heterocyclic group or an acyl group, or are optionally bonded to each other to form a non-aromatic ring, and other symbols are as defined above.

As the “optionally substituted hydrocarbon group”, “optionally substituted heterocyclic group” and “acyl group” for Re¹³ or Re¹⁴, those exemplarily recited as the “optionally substituted hydrocarbon group”, “optionally substituted heterocyclic group” and “acyl group”, which are those exemplarily recited as the “substituent” for Re¹, Re², Re³, Re⁴, Re⁵, Re⁶ or Re⁷, can be mentioned.

As the “non-aromatic ring” formed by Re¹³ and Re¹⁴ bonded to each other, a non-aromatic heterocycle, which is exemplarily recited as the “substituent” for Re¹, Re², Re³, Re⁴, Re⁵, Re⁶ or Re⁷, can be mentioned.

The synthesis of compounds Ie-XII, Ie-XIII and Ie-XIV is presented in Scheme 9. Compounds Ie-XII can be prepared by reacting a suitably protected (preferably Boc) amino group substituted amine EVIb with a previously described carbamate EVb under conditions mentioned in Scheme 4, followed by deprotection of the amino group. Compounds Ie-XII or their salts can be further transformed to the corresponding substituted amines (e.g., amides, sulfonamides, alkyl, aryl, heteroaryl amines or ureas) (Ie-XIII) under conditions commonly employed such as those described in Scheme 7. In the case of amides, the polymer supported conditions described in Scheme 8 can be applied toward the synthesis of amide libraries. Compounds Ie-XIII can be further transformed to Ie-XIV under conditions commonly employed such as those described in Scheme 7.

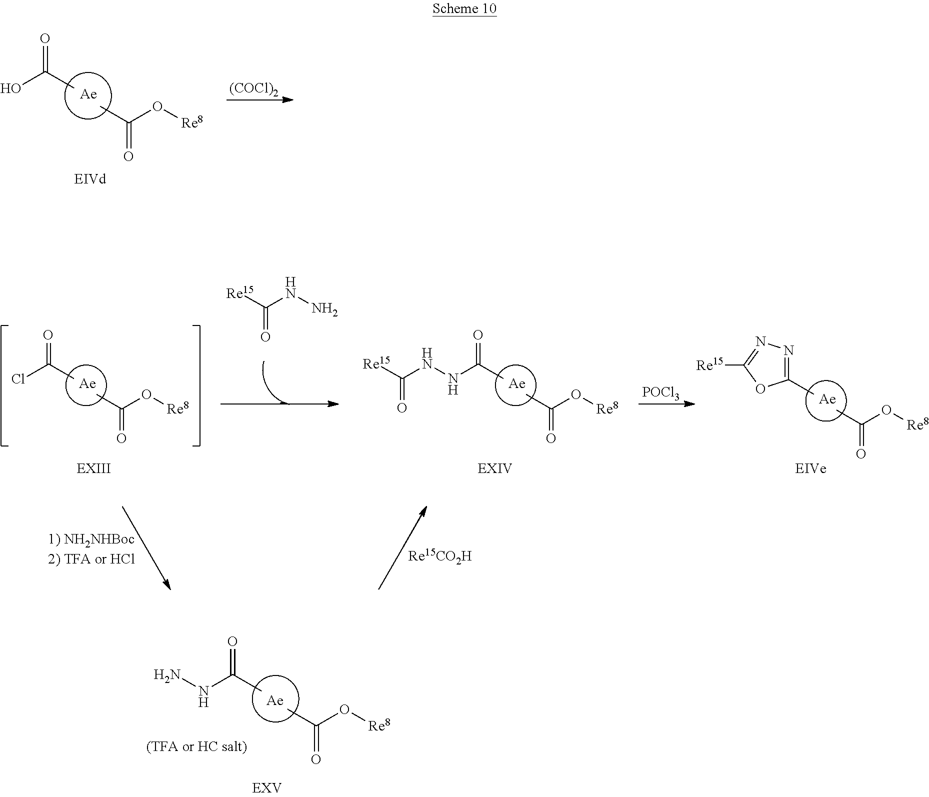

wherein Re¹⁵ is an optionally substituted hydrocarbon group or an optionally substituted heterocyclic group and other symbols are as defined above.

As the “optionally substituted hydrocarbon group” and “optionally substituted heterocyclic group” for Re¹⁵, those exemplarily recited as the “optionally substituted hydrocarbon group” and “optionally substituted heterocyclic group”, which are those exemplarily recited as the “substituent” for Re¹, Re², Re³, Re⁴, Re⁵, Re⁵ or Re⁷, can be mentioned.

Scheme 10 shows methods of preparing intermediates, in which the Ae ring is substituted with a 1,3,4-oxadiazolyl group. Compounds EIVe are suitable for use in preparing compounds of formulas Ie and Ie-I as shown in Schemes 2, 3 and

Compounds EXIII can be prepared by treating an acid EIVd with a reagent such as thionyl chloride or oxalyl chloride (1.1-1.5 equivalents) in a solvent such as halogenated hydrocarbons with a catalytic amount of dimethylformamide (0.01 equivalents) for 30 minutes to 1 hour at a temperature from 0° C. to 50° C. The resulting acid chloride EXIII can be treated with an acyl hydrazide (R¹⁵CONHNH₂) (1.1-1.5 equivalents) in solvents such as halogenated hydrocarbons in the presence of a base such as triethylamine (1.5-3 equivalents) for 1 to 48 hours at a temperature from 0° C. to 25° C. Compounds EXIV can be treated with a reagent such as POCl₃, PhPOCl₂, TFAA/Py or TsCl/Py to form the corresponding substituted 1,3,4-oxadiazoles EIVe. Preferably, compounds EXIV are treated with POCl₃ (1.1-10 equivalents) in a solvent such as acetonitrile or neat at a temperature of 80° C. for 1 to 24 hours.

Another method consists in treating the acid chloride EXIII with a suitably protected hydrazine in a solvent such as halogenated hydrocarbons, THF or benzene in the presence of a base such as triethylamine or diisopropylamine at a temperature from 20° C. to 90° C. for 1 to 24 hours. In the present invention, the protected hydrazine is preferably Boc-hydrazine (1.1-1.5 equivalents). The resulting protected hydrazide may be hydrolyzed in acidic conditions such as TFA or HCl in dioxane (2-10 equivalents) at temperatures from 20° C. to 90° C. for 1 to 24 hours to yield the hydrazide salts EXV. Compounds EXV can be reacted with the corresponding acid under conditions commonly used to form amide bonds to give compounds EXIV, which can be converted to compounds EIVe under the conditions mentioned above. Compounds EIVe may be hydrolyzed to the corresponding acids under conditions commonly employed.

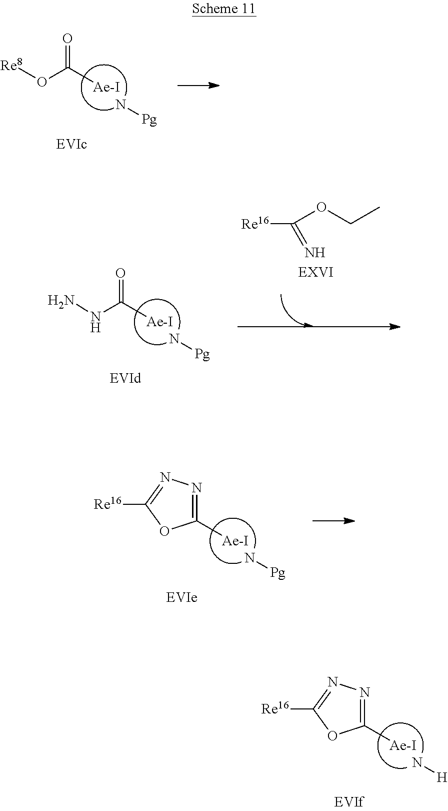

wherein Re¹⁶ is an optionally substituted hydrocarbon group or an optionally substituted heterocyclic group and other symbols are as defined above.

As the “optionally substituted hydrocarbon group” and “optionally substituted heterocyclic group” for Re¹⁶, those exemplarily recited as the “optionally substituted hydrocarbon group” and “optionally substituted heterocyclic group”, which are those exemplarily recited as the “substituent” for Re¹, Re², Re³, Re⁴, Re⁵, Re⁶ or Re⁷, can be mentioned.

Scheme 11 shows methods of preparing intermediates, in which the Ae-I ring is substituted with a 1,3,4-oxadiazolyl group. Compounds EVIf are suitable for use in preparing compounds of formulas Ie-II, Ie-IV and Ie-V as shown in Schemes 4, 5 and 6.

Compounds EVIe can be prepared by treating an ester EVIc with hydrazine (10-50 equivalents) at a temperature of 120° C. followed by reaction with a suitably substituted C₁-C₄ alkyl imidate EXVI (1.5-3 equivalents) in polar protic solvents such as alcohols at temperatures ranging from 80° C. to 130° C. for 1 to 48 hours. Compounds EVIe can be converted to compounds EVIf by deprotection of the amino group under conditions commonly employed.

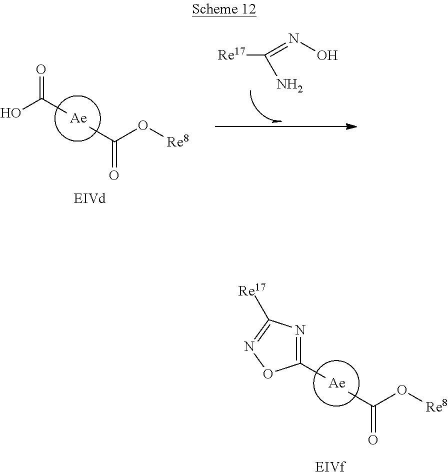

wherein Re¹⁷ is an optionally substituted hydrocarbon group or an optionally substituted heterocyclic group and other symbols are as defined above.

As the “optionally substituted hydrocarbon group” and “optionally substituted heterocyclic group” for Re¹⁷, those exemplarily recited as the “optionally substituted hydrocarbon group” and “optionally substituted heterocyclic group”, which are those exemplarily recited as the “substituent” for Re¹, Re², Re³, Re⁴, Re⁵, Re⁶ or Re⁷, can be mentioned.

Scheme 12 shows methods of preparing intermediates, in which the Ae ring is substituted with a 1,2,4-oxadiazolyl group. Compounds EIVf are suitable for use in preparing compounds of formulas Ie and Ie-I as shown in Schemes 2, 3 and 5.

Compounds EIVf can be prepared from an acid EIVd or the corresponding acid chloride and the corresponding substituted hydroxyamidine or its salt in a presence of a reagent such as a base, CDI, DCC/benzotriazole, DCC or TFFH in solvents such as DMF, THF, ACN or halogenated hydrocarbons. In the present invention, the acid EIVd is preferably treated with TFFH (1-1.5 equivalents) in the presence of a base such as triethylamine or diisopropylethylamine (1-1.5 equivalents) in a solvent such as DMF for 1 to 3 hours, and then treated with the corresponding substituted hydroxyamidine (1-1.5 equivalents) or its salt in a solvent such as DMF for 4 to 24 hours at a temperature of 100° C. according to the procedure described in J. Med. Chem. 2004, 12, 3111. Compounds EIVf can be hydrolyzed to the corresponding acids under conditions commonly employed.

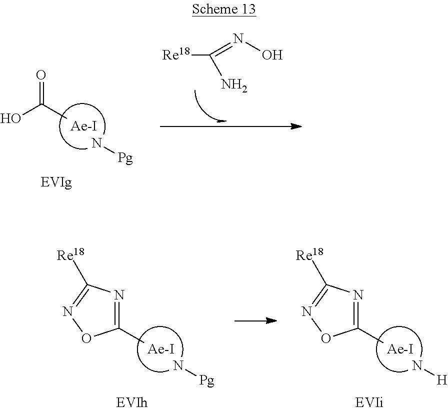

wherein Re¹⁸ is an optionally substituted hydrocarbon group or an optionally substituted heterocyclic group and other symbols are as defined above.

As the “optionally substituted hydrocarbon group” and “optionally substituted heterocyclic group” for Re¹⁸, those exemplarily recited as the “optionally substituted hydrocarbon group” and “optionally substituted heterocyclic group”, which are those exemplarily recited as the “substituent” for Re¹, Re², Re³, Re⁴, Re⁵, Re⁶ or Re⁷, can be mentioned.

Scheme 13 shows methods of preparing intermediates, in which the Ae-I ring is substituted with a 1,2,4-oxadiazolyl group. Compounds EVli are suitable for use in preparing compounds of formulas Ie-II, Ie-IV and Ie-V as shown in Schemes 4, 5 and 6.

Compounds EVlh can be prepared by treating an acid EVlg in similar conditions to those described in Scheme 12. Compounds EVIh can be converted to compounds EVIi by deprotection of the amino group under conditions commonly employed.

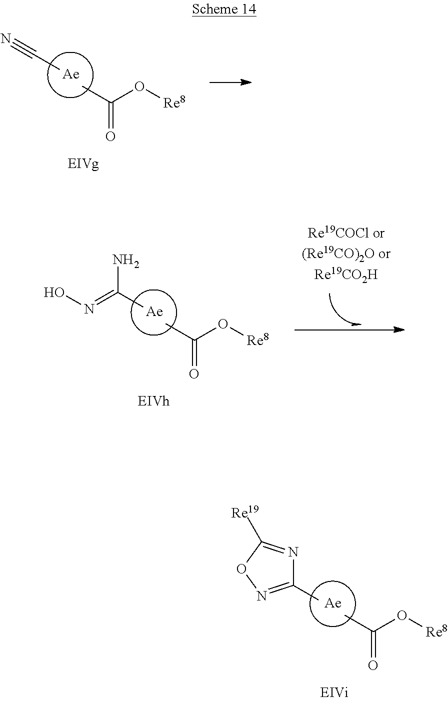

wherein Re¹⁹ is an optionally substituted hydrocarbon group or an optionally substituted heterocyclic group and other symbols are as defined above.

As the “optionally substituted hydrocarbon group” and “optionally substituted heterocyclic group” for Re¹⁹, those exemplarily recited as the “optionally substituted hydrocarbon group” and “optionally substituted heterocyclic group”, which are those exemplarily recited as the “substituent” for Re¹, Re², Re³, Re⁴, Re⁵, Re⁶ or Re⁷, can be mentioned.

Scheme 14 shows methods of preparing intermediates, in which the Ae ring is substituted with a 1,2,4-oxadiazolyl group. Compounds EIVi are suitable for use in preparing compounds of formulas Ie and Ie-I as shown in Schemes 2, 3 and 5.

Compounds EIVi can be prepared by treating a nitrile EIVg (prepared from the corresponding acid following the procedure described in J. Am. Chem. Soc. 1960, 82, 2457) with hydroxylamine hydrochloride (1 equivalent) in solvents such as aqueous alcohol (ethanol) at a temperature of 0° C. to reflux for 1 to 24 hours and reacting the hydroxylamine EIVh with either the corresponding acid chloride or acid anhydride (1.2-2 equivalents) in the presence of a base (1.5 to 3 equivalents), or the corresponding acid in the presence of TFFH as described in Schemes 12 and 13. Compounds EIVi can be hydrolyzed to the corresponding acids under conditions commonly employed.

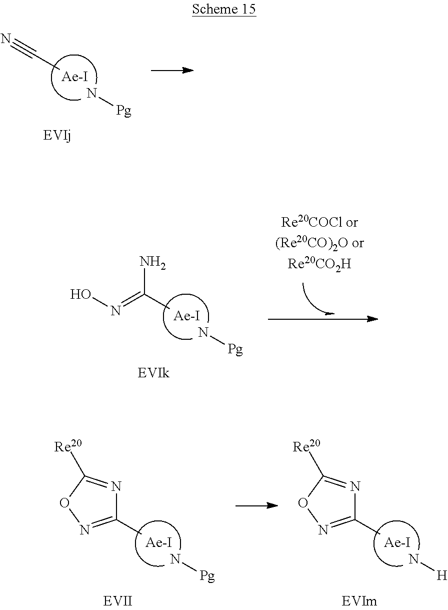

wherein Re²⁰ is an optionally substituted hydrocarbon group or an optionally substituted heterocyclic group and other symbols are as defined above.

As the “optionally substituted hydrocarbon group” and “optionally substituted heterocyclic group” for Re²⁰, those exemplarily recited as the “optionally substituted hydrocarbon group” and “optionally substituted heterocyclic group”, which are those exemplarily recited as the “substituent” for Re¹, Re², Re³, Re⁴, Re⁵, Re⁶ or Re⁷, can be mentioned.

Scheme 15 shows methods of preparing intermediates, in which the Ae-I ring is substituted with a 1,2,4-oxadiazolyl group. Compounds EVIm are suitable for use in preparing compounds of formulas Ie-II, Ie-IV and Ie-V as shown in Schemes 4, 5 and 6.

Compounds EVIl can be prepared by treating a suitably protected (Pg) amine EVIj in similar conditions to those described in Scheme 14. Compounds EVIl can be converted to compounds EVIm by deprotection of the amino group under conditions commonly employed.

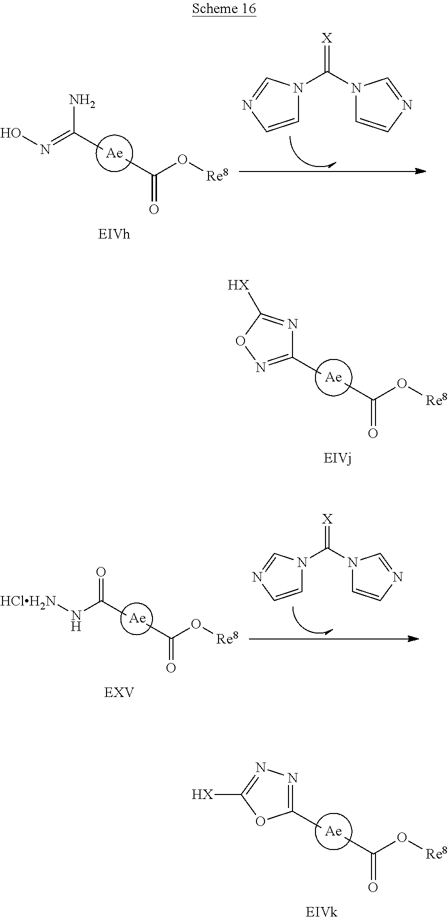

wherein X is an oxygen or a sulfur atom and other symbols are as defined above.

Scheme 16 shows methods of preparing intermediates, in which the Ae ring is substituted with a 5-mercapto (or hydroxy)-1,2,4-oxadiazolyl or 2-mercapto (or hydroxy)-1,3,4-oxadiazolyl group. Compounds EIVj and EIVk are suitable for use in preparing compounds of formulas Ie and Ie-I as shown in Schemes 2, 3 and 5

Compounds EIVj and EIVk can be prepared by treating an hydroxyamidine EIVh or the hydrazide salt EXV with 1,1′-carbonyldiimidazole (X═O) or 1,1′-thiocarbonyldiimidazole (X═S) in an aprotic solvent such as THF or dioxane at a temperature from 0° C. to 100° C. for 30 minutes to 24 hours. Typically 1 equivalent of EIVh or EXV, 1.2-1.5 equivalents of 1,1′-carbonyldiimidazole or 1,1′-thiocarbonyldiimidazole in 1,4-dioxane at reflux for 30 minutes to 3 hours. Compounds EIVj and EIVk can be hydrolyzed to the corresponding acids under conditions commonly employed.

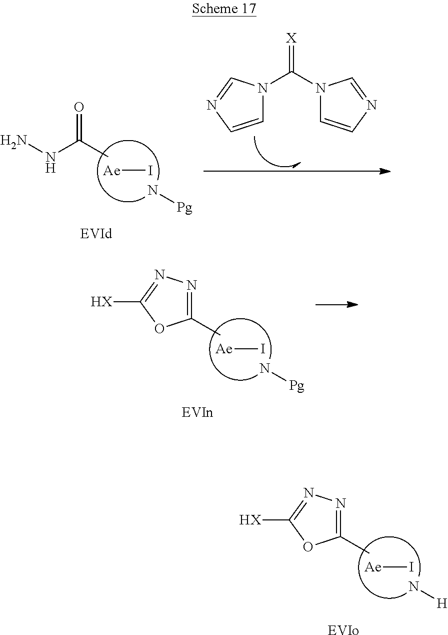

wherein the symbols are as defined above.

Scheme 17 shows methods of preparing intermediates, in which the Ae-I ring is substituted with a 2-mercapto (or hydroxy)-1,3,4-oxadiazolyl group. Compounds EVIo are suitable for use in preparing compounds of formulas Ie-II, Ie-IV and Ie-V as shown in Schemes 4, 5 and 6

Compounds EVIn can be prepared by treating a suitably protected (Pg) amine EVId in similar conditions to those described in Scheme 16. Compounds EVIn can be converted to compounds EVIo by deprotection of the amino group under conditions commonly employed.

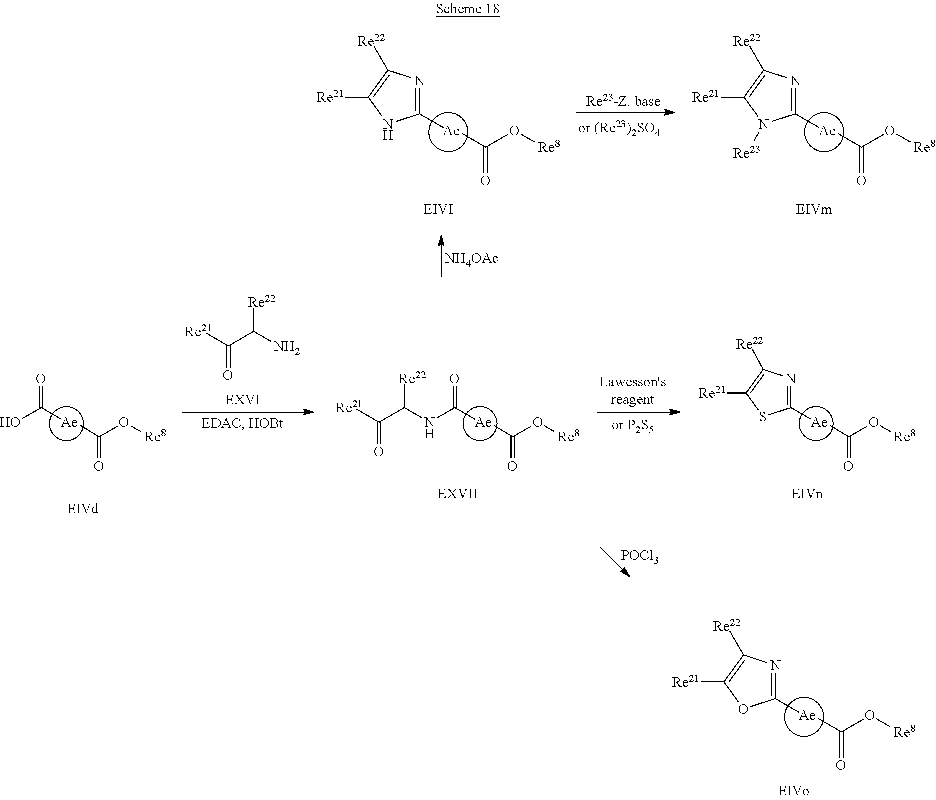

wherein Re²¹ and Re²² are each independently a hydrogen atom, an optionally substituted hydrocarbon group or an optionally substituted heterocyclic group, Re²³ is an optionally substituted C₁-C₄ alkyl group, Z is a leaving group such as a halogen atom or a substituted sulfonyloxy group and other symbols are as defined above.

As the “optionally substituted hydrocarbon group” and “optionally substituted heterocyclic group” for Re²¹ or Re²², those exemplarily recited as the “optionally substituted hydrocarbon group” and “optionally substituted heterocyclic group”, which are those exemplarily recited as the “substituent” for Re¹, Re², Re³, Re⁴, Re⁵, Re⁶ or Re⁷, can be mentioned.

As the “optionally substituted C₁-C₄ alkyl group” for Re²³, C₁-C₄ alkyl group optionally substituted by 1 to 3 substituents selected from a halogen atom, a carboxy group, a hydroxy group, a C₁₋₆ alkoxy-carbonyl group, a C₁₋₆ alkyl-carbonyloxy group (e.g., acetyloxy), a carbamoyl group, a cyano group, and a non-aromatic heterocyclic group (e.g., morpholinyl) can be mentioned.

Scheme 18 shows methods of preparing intermediates, in which the Ae ring is substituted with an imidazolyl, thiazolyl or oxazolyl group. Compounds EIVl, EIVm, EIVn and EIVo are suitable for use in preparing compounds of formulas Ie and Ie-I as shown in Schemes 2, 3 and 5.

Amides EXVII can be prepared by reaction of an acid EIVd with an optionally substituted O-keto amine EXVI under conditions commonly used for amide bond formation. Imidazole EIVl can be prepared by following the procedure reported in J. Med. Chem. 2004, 47(9), 2318. In the present invention, imidazole EIVl is preferably obtained by treating an amide EXVII with NH₄OAc. Imidazoles EIVl can be alkylated under various conditions including, but not limited to, treatment with the corresponding alkyl halide or sulfonate in the presence of an inorganic base such as sodium hydride, potassium carbonate or potassium hydroxide in the presence of a solvent such as DMSO, DMF or acetone at a temperature from 0° C. to 180° C. for 1 to 48 hours. When R²³ is a methyl group, imidazoles EIVl may conveniently be treated with dimethylsulfate (1.1 equivalents) in acetone for 1 to 48 hours at refluxing temperature to afford compound EIVm. Thiazoles EIVn can be prepared by treating an intermediate EXVII with Lawesson's reagent (for a description of Lawesson's reagent and its use, see Tetrahedron 1985, 41, 5061) or similar reagent (0.2-0.8 equivalents) in a solvent such as THF, toluene or xylene at temperatures from 20° C. to 150° C. for 1 to 24 hours. P₂S₅ in the presence of pyridine at a temperature of 100° C. for 30 minutes to 8 hours can also accomplish the desired transformation. Oxazoles EIVo can be prepared by treating an intermediate EXVII with a dehydrating agent such as POCl₃, POBr₃, PCl₅ or PhPOCl₂ neat or in a solvent such as toluene, acetonitrile or DMF at temperatures from 20° C. to 140° C. for 1 to 24 hours. Compounds EIVl, EIVm, EIVn and EIVo can be hydrolyzed to the corresponding acids under conditions commonly employed.

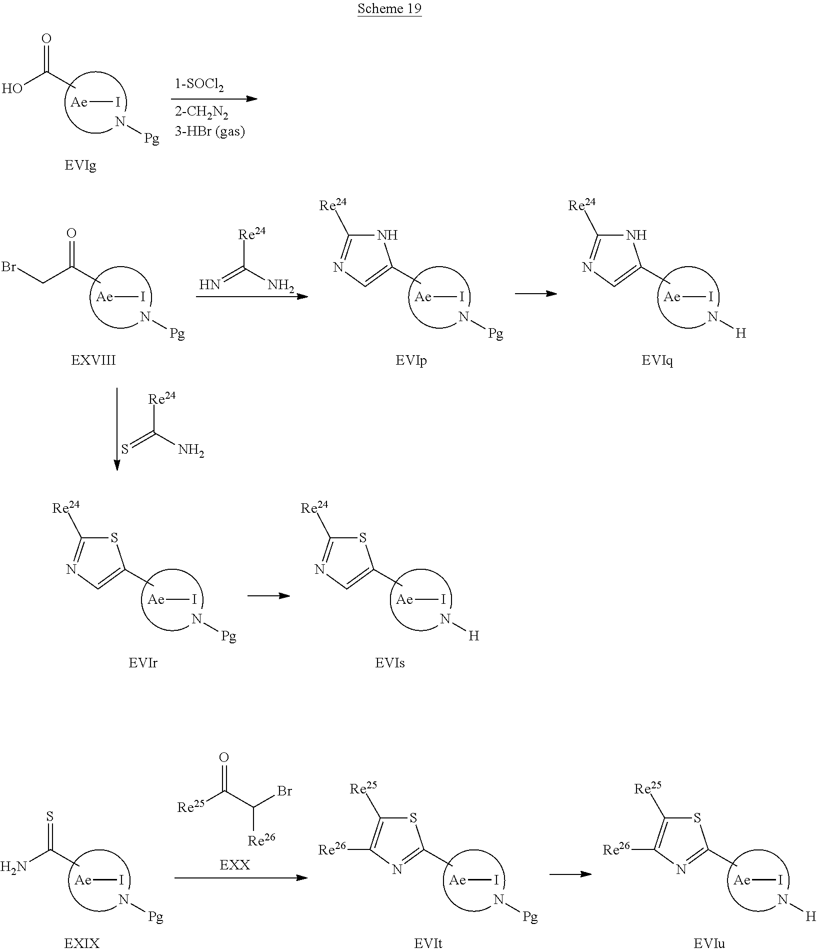

wherein Re²⁴ is an optionally substituted hydrocarbon group or an optionally substituted heterocyclic group, Re²⁵ and Re²⁶ are each independently a hydrogen atom, an optionally substituted hydrocarbon group or an optionally substituted heterocyclic group and other symbols are as defined above.

As the “optionally substituted hydrocarbon group” and “optionally substituted heterocyclic group” for Re²⁴, Re²⁵ or Re²⁶, those exemplarily recited as the “optionally substituted hydrocarbon group” and “optionally substituted heterocyclic group”, which are those exemplarily recited as the “substituent” for Re¹, Re², Re³, Re⁴, Re⁵, Re⁶ or Re⁷, can be mentioned.

Alternatively, Scheme 19 shows methods of preparing intermediates, in which the Ae-I ring is substituted with a thiazolyl or imidazolyl group. Compounds EVIq, EVIs and EVIu are suitable for use in preparing compounds of formulas Ie-IV and Ie-V as shown in Schemes 4, 5 and 6.

Acids EVIg can be elaborated to the α-bromoketones EXVIII which can be reacted with the corresponding amidine to afford the imidazoles EVIp following a procedure described in Bioorg. Med. Chem. Lett., 2004, 14, 3419. α-Bromoketones EXVIII may also be treated with the corresponding thioamide to afford the thiazols EVIr. Thiazole isomers EVIt can be prepared by treating thioamides EXIX with optionally substituted α-bromoketones EXX neat at a temperature of 80° C. to 170° C. or in a solvent such as ethanol or DMF at a temperature of 80° C. to 130° C. for 10 minutes to 24 hours. Compounds EVip, EVIr and EVIt can be converted to compounds EVIq, EVIs and EVIu by deprotection of the amino group under conditions commonly employed.

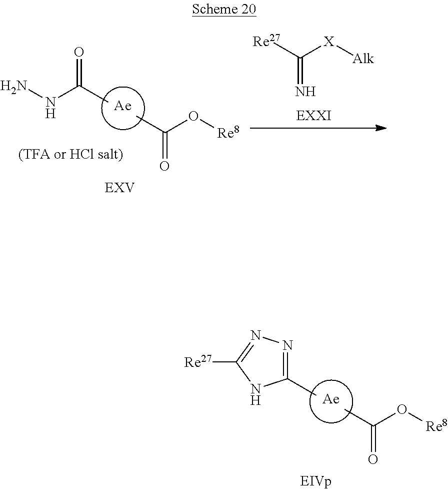

wherein Re²⁷ is an optionally substituted hydrocarbon group or an optionally substituted heterocyclic group, Alk is a C₁-C₆ alkyl group and other symbols are as defined above.

As the “optionally substituted hydrocarbon group” and “optionally substituted heterocyclic group” for Re²⁷, those exemplarily recited as the “optionally substituted hydrocarbon group” and “optionally substituted heterocyclic group”, which are those exemplarily recited as the “substituent” for Re¹, Re², Re³, Re⁴, Re⁵, Re⁶ or Re⁷, can be mentioned.

Scheme 20 shows methods of preparing intermediates, in which the Ae ring is substituted with a triazolyl group. Compounds EIVp are suitable for use in preparing compounds of formulas Ie and Ie-I as shown in Schemes 2, 3 and 5.

Compounds EIVp may be prepared from an optionally substituted hydrazide EXV by treatment with the corresponding alkyl (preferably ethyl or methyl) imidate (X═O, prepared via the Pinner reaction) or imidothioate (X═S) in a solvent such as DMF at a temperature of 70° C. to 150° C. for 1 to 24 hours. Hydrazides EXV can also be treated with the corresponding amidines, thioamides or nitriles in the presence of organic or inorganic bases in polar protic solvents such as alcohols. Typically, hydrazides EXV are treated with an ethyl imidate (1.2-2 equivalents) in refluxing ethanol. Compounds EIVp can be hydrolyzed to the corresponding acids under conditions commonly employed.

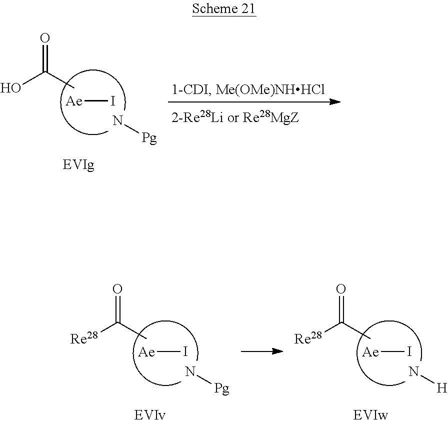

wherein Re²⁸ is an optionally substituted hydrocarbon group or an optionally substituted heterocyclic group and other symbols are as defined above.

As the “optionally substituted hydrocarbon group” and “optionally substituted heterocyclic group” for Re²⁸, those exemplarily recited as the “optionally substituted hydrocarbon group” and “optionally substituted heterocyclic group”, which are those exemplarily recited as the “substituent” for Re¹, Re², Re³, Re⁴, Re⁵, Re⁶ or Re⁷ can be mentioned.

Scheme 21 shows methods of preparing intermediates, in which the Ae-I ring is substituted with a substituted carbonyl group. Compounds EVIw are suitable for use in preparing compounds of formulas Ie-II, Ie-IV and Ie-V as shown in Schemes 4, 5 and 6.

Acids EVIg can be converted to the corresponding Weinreb amides by treatment with CDI in a solvent such as DCM followed by addition of N-methoxy-N-methylamine hydrochloride and a base such as triethylamine or diisopropylethylamine. The corresponding alkyl halide, aryl halide or halogenated heterocycle, can be first metalated with an alkyl lithium reagent such as n-butyl, sec-butyl or tert-butyl lithium, or the corresponding Grignard reagent such as, i-PrMg-halide then reacted with the previously prepared Weinreb amide in a polar aprotic solvent such as THF at a temperature from −78° C. to 80° C. for 1 to 24 hours to afford the corresponding carbonyl substituted intermediate EVIv accordingly to the procedures described in US2002/0151717 and U.S. Pat. No. 6,465,490. Compounds EVIv can be converted to compounds EVIw by deprotection of the amino group under conditions commonly employed.

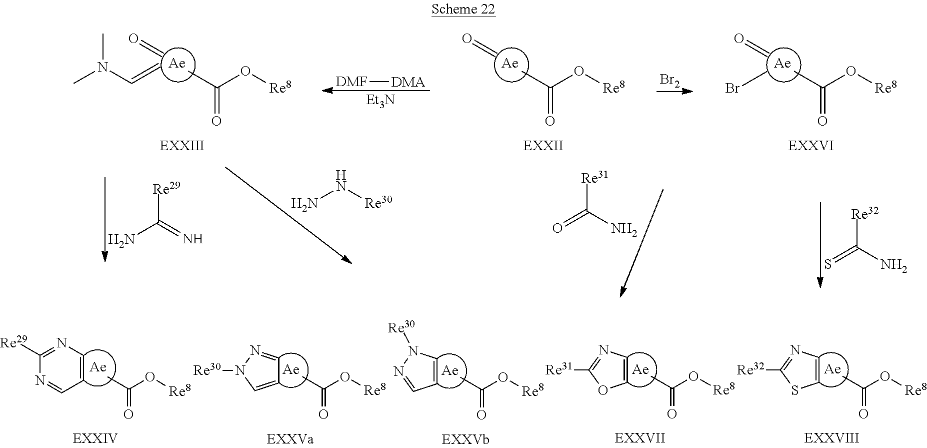

wherein Re²⁹, Re³⁰, Re³¹ and Re³² are each independently a hydrogen atom, an optionally substituted hydrocarbon group or an optionally substituted heterocyclic group and other symbols are as defined above.

As the “optionally substituted hydrocarbon group” and “optionally substituted heterocyclic group” for Re²⁹, Re³⁰, Re³¹ or Re³², those exemplarily recited as the “optionally substituted hydrocarbon group” and “optionally substituted heterocyclic group”, which are those exemplarily recited as the “substituent” for Re¹, Re², Re³, Re⁴, Re⁵, Re⁶ or Re⁷, can be mentioned.

Scheme 22 shows methods of preparing intermediates, in which the Ae ring is an optionally substituted non-aromatic fused heterocyclic ring. Compounds EXXIV, EXXVa, EXXVb, EXXVII and EXXVIII are suitable for use in preparing compounds of formulas Ie and Ie-I as shown in Schemes 2, 3 and 5.

(Dimethylamino)methylene ketones EXXIII can be prepared by treating cyclic ketones EXXII with dimethylformamide dimethylacetal in the presence of triethylamine at 100° C. for 24 hours. Compounds EXXIII can then either be reacted with the corresponding substituted amidine or its salt in a polar protic solvent such as alcohols at a temperature of 60° C. to 80° C. for 4 to 24 hours to afford the fused pyrimidines EXXIV, or be reacted with the corresponding substituted hydrazine or its salts in similar conditions to afford the fused pyrazole isomers EXXVa and EXXVb. α-Bromo ketones EXXVI can be prepared by reacting cyclic ketones EXXII with bromine in a solvent such as ethyl ether at a temperature from 0° C. to 20° C. for 1 to 24 hours according to a procedure similar to the one described in Eur. J. Med. Chem.—Chim. Ther., 1984, 19(5), 457. Compounds EXXVI can either be reacted with the corresponding optionally substituted amide or thioamide (prepared from the corresponding amide accordingly to Eur. J. Med. Chem. 2004, 39(10), 867-872) to afford respectively fused oxazoles EXXVII and fused thiazoles EXXVIII. The reaction is usually conducted neat with one equivalent of each reactant at a temperature from 60° C. to 160° C. for 10 minutes to 24 hours. A solvent such as ethanol or DMF and a base such as potassium carbonate or cesium carbonate may be added to facilitate the reaction. Compounds EXXIV, EXXVa, EXXVb, EXXVII and EXXVIII can be hydrolyzed to the corresponding acids under conditions commonly employed.

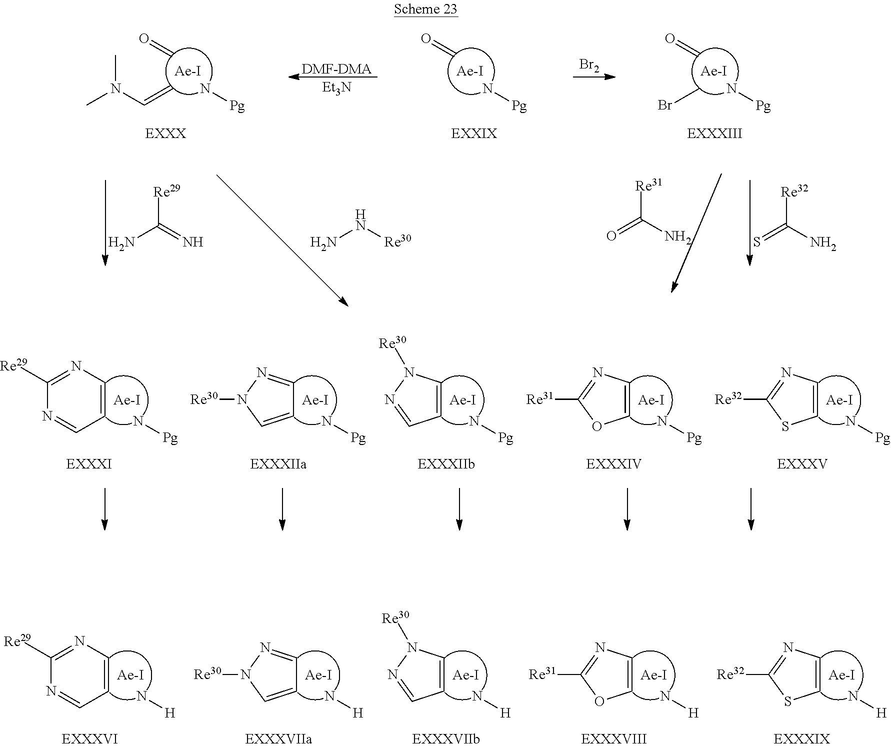

wherein the symbols are as defined above.

Scheme 23 shows methods of preparing intermediates, in which the Ae-I ring is an optionally substituted non-aromatic fused heterocyclic ring. Compounds EXXXVI, EXXXVIIa, EXXXVIIb, EXXXVIII and EXXXIX are suitable for use in preparing compounds of formulas Ie-II, Ie-IV and Ie-V as shown in Schemes 4, 5 and 6.

Pyrimidines EXXXI, pyrazoles EXXXIIa-b, oxazoles EXXXIV and thiazoles EXXXV can be prepared under similar conditions to those described in Scheme 22. Compounds EXXXI, EXXXIIa, EXXXIIb, EXXXIV and EXXXV can be converted to compounds EXXXVI, EXXXVIIa, EXXXVIIb, EXXXVIII and EXXXIX by deprotection of the amino group under conditions commonly employed.

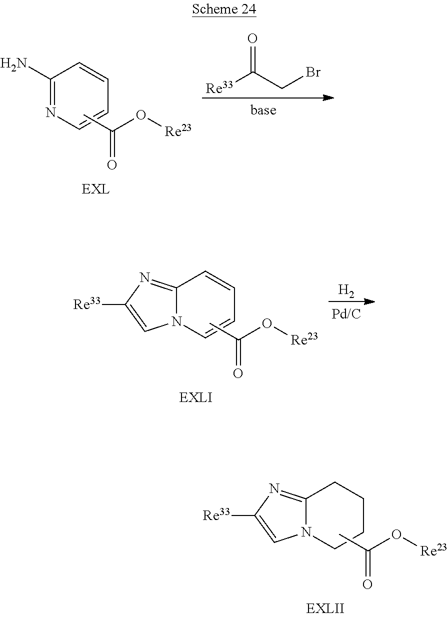

wherein Re³³ is an optionally substituted hydrocarbon group or an optionally substituted heterocyclic (aromatic or non-aromatic) group and other symbols are as defined above.

As the “optionally substituted hydrocarbon group” and “optionally substituted heterocyclic group” for Re³³, those exemplary recited as the “optionally substituted hydrocarbon group” and “optionally substituted heterocyclic group”, which are those exemplarily recited as the “substituent” for Re¹, Re², Re³, Re⁴, Re⁵, Re⁶ or Re⁷, can be mentioned.

Scheme 24 shows methods of preparing bicyclic intermediates. Compounds EXLII are suitable for use in preparing compounds of formula Ie and Ie-I as shown in Schemes 2, 3 and 5. An aminopyridine EXL may be treated with a corresponding optionally substituted α-bromo ketone in the presence of an inorganic base such as sodium bicarbonate in a polar protic solvent such as methanol or ethanol at a temperature of 40° C. to 80° C. for 4 to 24 hours to afford the imidazo[1,2-a]pyridine EXLI. Compounds EXLI may then be reduced to EXLII under hydrogen atmosphere in the presence of a catalyst such as palladium on carbon at a pressure of 12 to 400 psi, preferably 250 psi, at a temperature of 50° C. to 100° C. for 24 to 72 hours. Compounds EXLII can be hydrolyzed to the corresponding acids under conditions commonly employed.

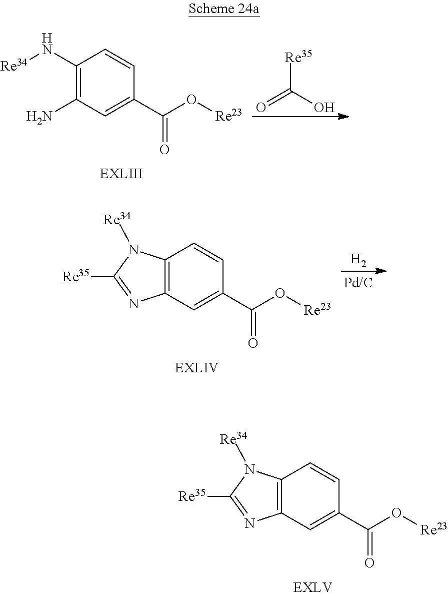

wherein Re³⁴ is a hydrogen atom or an optionally substituted hydrocarbon group, Re³⁵ is an optionally substituted hydrocarbon group or an optionally substituted heterocyclic (aromatic or non-aromatic) group and other symbols are as defined above.

As the “optionally substituted hydrocarbon group” for Re³⁴, those exemplarily recited as the “optionally substituted hydrocarbon group”, which are those exemplarily recited as the “substituent” for Re¹, Re², Re³, Re⁴, Re⁵, Re⁶ or Re⁷, can be mentioned.

As the “optionally substituted hydrocarbon group” and “optionally substituted heterocyclic group” for Re³⁵, those exemplarily recited as the “optionally substituted hydrocarbon group” and “optionally substituted heterocyclic group”, which are those exemplarily recited as the “substituent” for Re¹, Re², Re³, Re⁴, Re⁵, Re⁶ or Re⁷, can be mentioned.

Scheme 24a shows methods of preparing bicyclic intermediates. Compounds EXLV are suitable for use in preparing compounds of formula Ie and Ie-I as shown in Schemes 2, 3 and 5. Compound EXLIII may be treated with a corresponding optionally substituted carboxylic acid in the presence of an inorganic acid such HCl neat at a temperature of 40° C. to 100° C. for 4 to 24 hours to afford the 1H-benzo[d]imidazole EXLIV. Compounds EXLIV may then be reduced, to EXLV under hydrogen atmosphere in the presence of a catalyst such as palladium on carbon at a pressure of 12 to 400 psi, preferably 250 psi, at a temperature of 50° C. to 100° C. for 24 to 72 hours. Compounds EXLV can be hydrolyzed to the corresponding acids under conditions commonly employed.

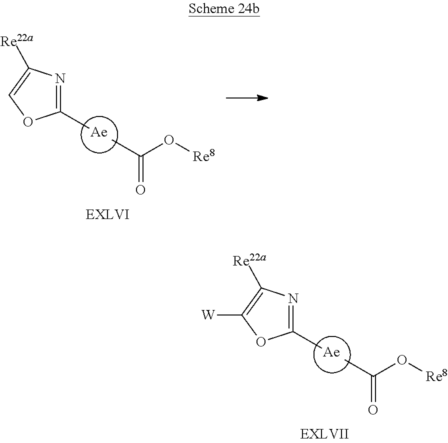

wherein Re^{22a} is an optionally substituted hydrocarbon group or an optionally substituted heterocyclic (aromatic or non-aromatic) group, W is a halogen atom, and other symbols are as defined above.

As the “optionally substituted hydrocarbon group” and “optionally substituted heterocyclic group” for Re^{22a}, those exemplarily recited as the “optionally substituted hydrocarbon group” and “optionally substituted heterocyclic group”, which are those exemplarily recited as the “substituent” for Re¹, Re², Re³, Re⁴, Re⁵, Re⁶ or Re⁷, can be mentioned.

Scheme 24b shows methods for preparing compounds of formula EXLVII where an oxazole ring is substituted with a halogen atom. Compounds EXLVII can be prepared from a halogenating source, such as bromine, in the presence of a solvent, such as DMF or dioxane. Solvents including halogenated hydrocarbons such as dichloromethane or 1,2-dichloroethane may be used as well. In some instances, an acid such as TFA or AcOH may be used. Likewise the reaction may also be performed under basic conditions as well (such as an alkali carbonate or alkali hydroxide base). The reaction is usually performed at temperatures ranging from 0° C. to refluxing conditions. An alternative halogenating source utilizes an N-halosuccinimide. The reaction is usually performed in a solvent, such as DMF or acetonitrile at temperatures ranging from 0° C. to 90° C. In the present invention, N-bromosuccinimide is preferably used to obtain a brominated analog of EXLVII.

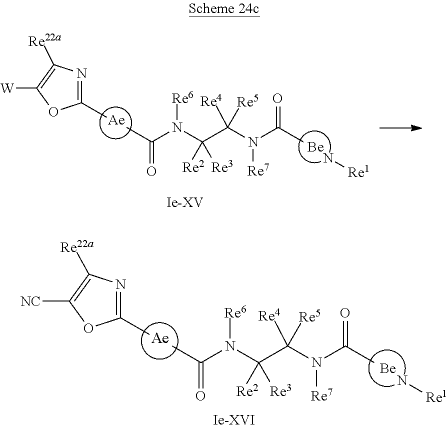

wherein the symbols are as defined above.

Scheme 24c shows methods for preparing compounds of formula Ie-XVI where an oxazole ring is substituted with a cyano group. Compounds Ie-XVI can be prepared by nucleophilic substitution in the presence of nitrile equivalents such as sodium cyanide, potassium cyanide or copper cyanide in solvents such as DMF or DMSO at temperatures from 80° C. to 180° C. for 1 to 48 hours. Compounds Ie-XVI can also be prepared from halides Ie-XV in the presence of zinc cyanide or potassium cyanide and a palladium catalyst such as Pd(PPh₃)₄ or Pd(OAc)₂ and a phosphine in solvents such as DMF at 80° C. to 160° C. for 1 to 24 hours (for a review on Pd-catalyzed cyanation of aryl halides see Eur. J. Inorg. Chem. 2003, 19, 3513). Typically, the reaction is performed using copper cyanide (1 to 2 equivalents) in a solvent such as DMF at a temperature from 130° C. to 160° C. for 16 to 48 hours.

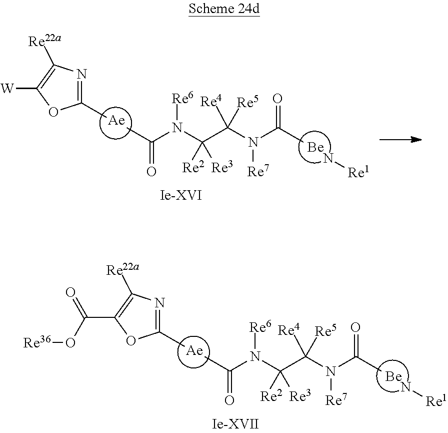

wherein Re³⁶ is a hydrogen atom or an optionally substituted C₁-C₄ alkyl group and other symbols are as defined above.

As the “optionally substituted C₁-C₄ alkyl group” for Re³⁶, those exemplarily recited as the “optionally substituted C₁-C₄ alkyl group” for Re²³ can be mentioned.

Scheme 24d shows methods for preparing compounds of formula Ie-XVII where an oxazole ring is substituted with a carboxylic ester or carboxylic acid group (Re³⁶═H). Compounds Ie-XVII can be prepared from halides Ie-XV using a palladium-ligand catalyst such as (R)-(Binap)PdCl₂, Pd(OAc)₂ or PdCl₂(PPh₃)₂ in the presence of a base such as triethylamine, Hunig's base, alkali carbonates or alkali hydroxides (lithium hydroxide, sodium hydroxide or potassium hydroxide) in solvents such as toluene or alcohols under carbon monoxide atmosphere (J. Organomet. Chem. 2002, 641 (1-2), 30; Synthesis 2002, 15, 2171). In the present invention, halides Ie-XV are preferably treated with Pd(OAc)₂ (0.01 to 0.1 equivalents), 1,3-bis(diphenylphosphino)propane (0.02 to 0.2 equivalents), and K₂CO₃ (1 equivalent) in alcohol (preferably methanol or ethanol) under carbon monoxide pressure (15-100 psi) at a temperature from 20° C. to 100° C. for 24 to 48 hours to obtain the carboxylic ester. In the case of preparing the carboxylic acid Ie-XVII (Re³⁶═H), excess K₂CO₃ (2 to 5 equivalents) is added in a solvent mixture containing alcohol and water, preferably in a 9:1 ratio. Alternatively, carboxylic acid Ie-XVII (Re³⁶═H) can be prepared from a corresponding ester by alkali hydrolysis under conditions commonly employed.

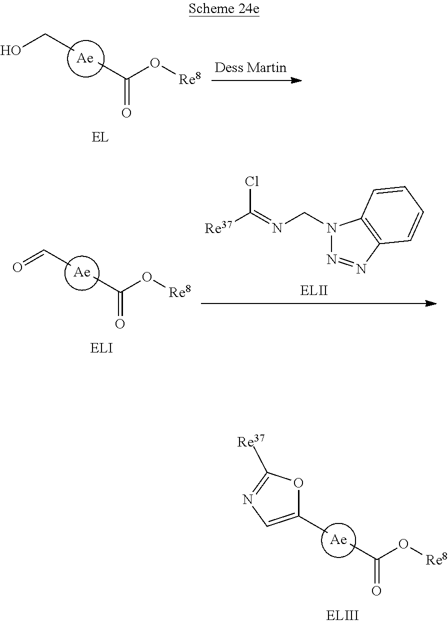

wherein Re³⁷ is an optionally substituted aromatic hydrocarbon group or an optionally substituted aromatic heterocyclic group and other symbols are as defined above.

As the “optionally substituted hydrocarbon group” and “optionally substituted heterocyclic group” for Re³⁷, those exemplarily recited as the “optionally substituted hydrocarbon group” and “optionally substituted heterocyclic group”, which are those exemplarily recited as the “substituent” for Re¹, Re², Re³, Re⁴, Re⁵, Re⁶ or Re⁷ can be mentioned.

Scheme 24e shows a method for preparing the compounds of formula ELIII which contain substituted oxazoles linked to the Ae ring. Alcohols of formula EL may be oxidized using a variety of conditions. Dess Martin Periodinane, Swern (DMSO, oxayl chloride), Collins (CrO₃.pyr), and NaOCl/Tempo represent a few oxidation conditions to form the aldehyde ELI from EL. The preferred conditions to prepare ELI use Dess Martin Periodinane (1.0 equivalent) in dichloromethane. The reaction is typically run at room temperature for 1 to 24 hours. ELIII can be prepared via a 1,3-dipolar cycloaddition of aldehydes ELI with azomethine generated from compounds of formula ELII with added base (Katritzky, J. Het. Chem. 2002, 39, 759-765). Typically, aldehyde ELI (1 equivalent) is added to the chloroimine ELII (1 equivalent) followed by base, preferably potassium t-butoxide (4 equivalents). The reaction is performed at −40° C. with warming to temperatures ranging from 22° C. to 75° C. THF is the preferred solvent of choice. Alternatively, oxazoles of formula ELIII can be prepared by acid catalyzed cyclization of β-ketoamides, using SOCl₂, POCl₃, etc., utilizing the Robinson-Gabriel oxazole synthesis (for example, see Litak. J. Het. Chem. 1994, 31, 457). The cyclization can be performed neat or in a solvent such as acetonitrile at a temperature of 80° C. for 1 to 24 hours. Compounds ELIII can be hydrolyzed to the corresponding acids under conditions commonly employed.

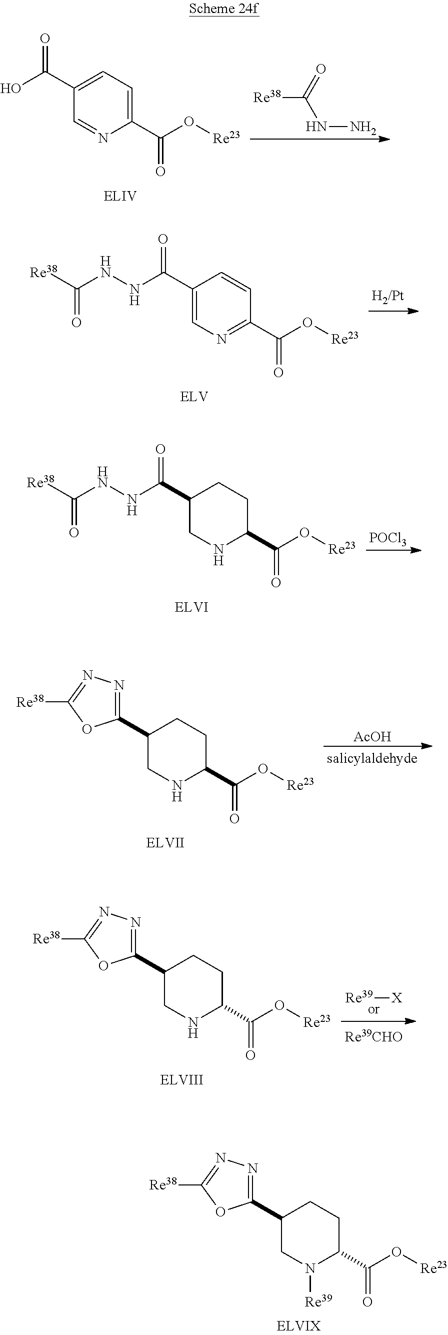

wherein Re³⁸ and Re³⁹ are each independently an optionally substituted hydrocarbon group or an optionally substituted heterocyclic (aromatic or non-aromatic) group and other symbols are as defined above.

As the “optionally substituted hydrocarbon group” and “optionally substituted heterocyclic group” for Re³⁸ or Re³⁹, those exemplarily recited as the “optionally substituted hydrocarbon group” and “optionally substituted heterocyclic group”, which are those exemplarily recited as the “substituent” for Re¹, Re², Re³, Re⁴, Re⁵, Re⁶ or Re⁷, can be mentioned.

Scheme 24f details the preparation of piperidine substituted with oxadiazoles of formula ELVIX. The preparation of compounds of formula ELVIX starts from ELIV, prepared using a procedure from U.S. Pat. No. 4,273,779. ELIV is coupled to a corresponding acyl hydrazine using standard amide coupling conditions as described previously. The preferred method employs the use of EDAc.HCl/HOBt.H₂O as the coupling reagent to form compounds of formula ELV. Reduction of the pyridine ring to the cis-piperidine ring occurs selectivity using a hydrogen atmosphere in the presence of platinum oxide (0.05 to 0.2 equivalents). The hydrogenation is typically performed at 20° C. at a pressure of 50 psi for 3 to 48 hours giving ELVI. The reaction is performed in a solvent such as acetic acid or an alcohol, preferably methanol or ethanol. Alternative methods for reduction to the piperidine ring include hydrogenations using catalytic palladium, Raney nickel, ruthenium oxide, and the like.

Diacyl hydrazines of formula ELVI can be cyclized using POCl₃, PhPOCl₂, TFAA/Py or TsCl/Py to form the corresponding substituted 1,3,4-oxadiazoles ELVII. Compound ELVII can be epimerized by heating in acid or base to form a mixture of cis and trans isomers of formula ELVIII. The trans isomers can be isolated by silica gel column chromatography. The preferred method entails compound ELVII in acetic acid at reflux in the presence of catalytic salicylaldehyde (0.01 to 0.10 equivalents) for 3 to 24 hours according to the method of Urban, J. Het. Chem. 1995, 32, 857. Alkylation or arylation of ELVIII may afford compounds of formula ELVIX using standard alkylation or reductive amination conditions as described previously. N-arylation with an aryl halide may also occur using palladium catalyzed coupling conditions as described by Buchwald (J. Org. Chem. 2000, 65, 1144-1157). Compounds ELVIX can be hydrolyzed to the corresponding acids under conditions commonly employed.

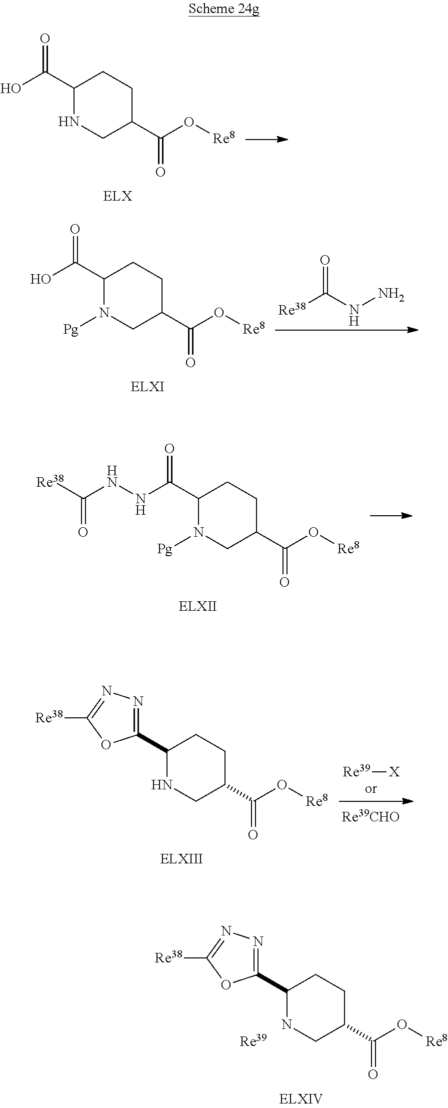

wherein the symbols are as defined above.

Scheme 24 g details the preparation of compounds of formula ELXIV. Piperidine acids of formula ELX may be obtained as a mixture of cis:trans isomers using the procedure of Urban, J. Het. Chem. 1995, 32, 857. Protection of the amine of ELX, preferably using di-tert-butyl dicarbonate when Pg is Boc) gives compounds of formula ELXI which are suitable for amide coupling. The preferred coupling utilizes EDAC.HCl/HOBt.H₂O with the appropriately substituted hydrazine to form compounds ELXII. Compounds of formula ELXII may then be cyclized to the oxadiazoles of formula ELXIII using POCl₃, PhPOCl₂, or other conditions previously described. The preferred condition uses POCl₃ (2.5 equivalents) in acetonitrile at reflux for 5 to 15 hours. Under these conditions, the Boc group is removed.

Subjecting ELXIII to alkylation, reductive amination, or N-arylation conditions as previously described affords compounds of formula ELXIV. Compounds ELXIV can be hydrolyzed to the corresponding acids under conditions commonly employed.

### General Synthetic Methods for Compound (If)

Compounds of formula If of this invention can be prepared by several methods generally known in the art of organic chemistry.

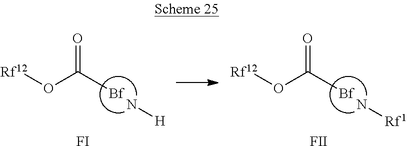

wherein Rf¹² is a C₁-C₄ alkyl or benzyl group and other symbols are as defined above.

Intermediate esters FII, which are suitable for use in preparing compounds of formulas If-I and If-II as shown in schemes 26 to 28, can be prepared under various conditions depending on the nature of the Rf¹ substituent.

In the case wherein Rf¹ is an optionally substituted aryl or heteroaryl group, esters FII can be prepared according to one of the following references: Tetrahedron Lett. 1998, 39, 2941-2944; Eur. J. Org. Chem. 2004, 695-709; Jr. Am. Chem. Soc 2001, 123, 7727-7729; J. Am. Chem. Soc. 2002, 124, 11684-11688; J. Org. Chem. 2004, 69, 5578. Typically, the N-arylation of the Bf ring is performed with the corresponding aryl halide (preferably iodide) (including a heteroaryl halide) or in the presence of copper catalyst such as copper iodide or copper oxide, in the presence of a ligand such as substituted ethylene diamines, salicylaldoximes or other ligands reported in Eur. J. Org. Chem. 2004, 695-709. The reaction requires a base such as potassium phosphate or cesium carbonate and is performed in a degassed solvent such as acetonitrile, toluene or DMF at a temperature of 20° C. to 150° C. for 0.5 to 48 hours under inert atmosphere. Preferably, the N-arylation is conducted according to the method described in J. Org. Chem. 2004, 69, 5578, in toluene with 1 equivalent of FI, 1.1-10 equivalents of aryl halide, 2 equivalents of diamine ligand, 2-3 equivalent of base and 0.05 equivalent of copper(I) iodide or according to the method described in Eur. J. Org. Chem. 2004, 695-709, in DMF with 1 equivalent of FI, 1.5-10 equivalents of aryl halide, 0.2-0.4 equivalents of oxime ligand, 2-3 equivalent of base and 0.05 equivalent of copper(II) oxide.

In the case where Rf¹ is an alkyl or cycloalkyl group, esters FII can be prepared by direct alkylation with the corresponding alkyl halide (including a cycloalkyl halide) or the corresponding alkyl sulfonate (including a cycloalkyl sulfonate) in the presence of a base such as potassium carbonate, cesium carbonate or sodium hydride in a solvent such as DMF at a temperature ranging from 20° C. to 130° C. for 0.5 to 48 hours. In the case of hindered or poorly reactive alkyl halides, the alkyl halide may be used as the solvent at a temperature ranging from 20° C. to 130° C. for 0.5 to 48 hours. Alternatively, esters FII can be prepared from the amine FI by opening of the corresponding epoxide in the presence of a base such as potassium or cesium carbonate in a solvent such as halogenated hydrochlorides or neat at a temperature from 20° C. to 100° C. for 1 to 48 hours. Preferably, the alkylation is run in DMF or halogenated hydrocarbons with 1 equivalent of FI, 1.1-10 equivalents of alkyl halide, alkyl sulfonate or epoxide and 1-5 equivalents of base.

In the case where Rf¹ is an acyl group, esters FII can be prepared with the corresponding acid halides or sulfonyl halides in the presence of a base such as sodium hydride, potassium carbonate, sodium hydroxide or triethylamine in a solvent such as DMF, acetone or halogenated hydrocarbons at a temperature ranging from 0° C. to 130° C. for 0.5 to 24 hours. Preferably, this reaction is run in DMF or halogenated hydrocarbons with 1 equivalent of FI, 1.1-2 equivalents sulfonyl or acyl halide and 1-5 equivalents of base.

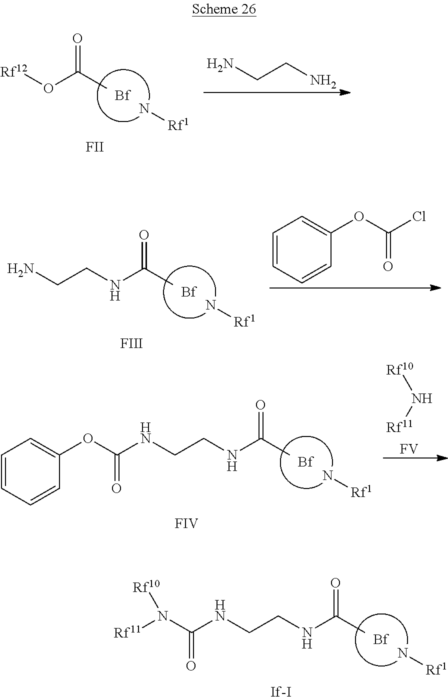

wherein the symbols are as defined above.

Compounds If-I, which is compound If wherein Yf is NH, can be prepared according to Scheme 26. Esters FII can be treated with ethylenediamine at refluxing temperature for 2 to 24 hours to produce amines FIII. Compounds FIV can be prepared by reacting an amine FIII with a chloroformate in an aprotic solvent such as halogenated hydrocarbons in the presence of a base such as triethylamine or pyridine at a temperature from 0° C. to 50° C. for 1 to 24 hours. In the present invention, the chloroformate of choice is phenyl chloroformate and the reaction is preferably run in dichloromethane at 20° C. for 2-3 hours in the presence of triethylamine. Compounds If-I are conveniently prepared by reacting carbamates FIV with an amine FV. The reaction is conducted neat or in a polar protic solvent such as ethanol at a temperature ranging from 120° C. to 170° C. for 15 minutes to 5 hours. A base such as potassium or cesium carbonate may be used. When the amine FV is used a as a salt, the reaction is conducted in a polar protic solvent preferably ethanol at a temperature ranging from 80° C. to 160° C. for 15 minutes to 5 hours in the presence of a base such as potassium carbonate, cesium carbonate, triethylamine, diisopropylethyl amine.

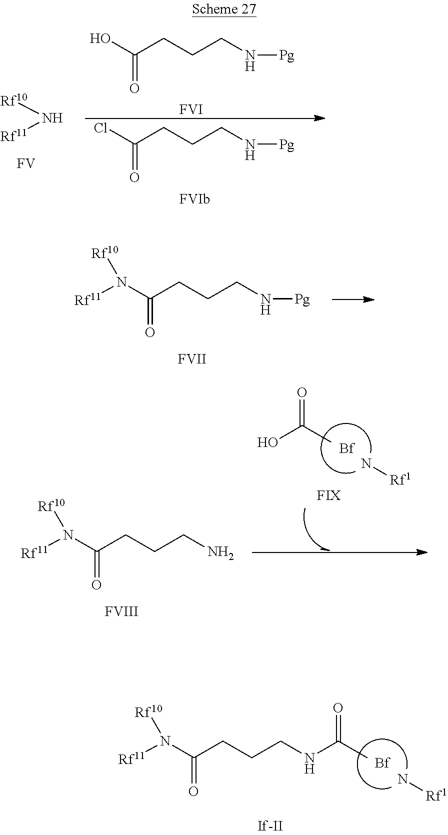

wherein Pg is a protecting group and other symbols are as defined above.

Compounds If-II, which is compound If wherein Yf is CH₂, can be prepared according to Scheme 27. Compounds of formula FVII can be conveniently prepared from a suitably protected acid FVI and amines FV or their salts by reacting both intermediates in the presence of various coupling reagents. Known reagents that effect amide bond formation include N,N-carbonyldiimidazole, halopyridine salts, 2,4,6-trichlorobenzoyl chloride, HATU, BOP—Cl or EDAC.HCl/HOBt.H₂O. In the present invention, the preferred reagent is EDAC.HCl/HOBt.H₂O. The reaction can be conducted in a variety of non-protic solvents such as, but not limited to, halogenated hydrocarbons, acetonitrile or dimethylformamide, or a mixture, at a temperature from 0° C. to 130° C., preferably 20° C. to 70° C., for a time ranging from 1 to 48 hours. A base such as triethylamine or diisopropylethylamine may be used especially if the reacting amine FV is in a salt form. While the amount of reagents vary depending on the coupling reagent used, the following amounts are used preferably with EDAC.HCl/HOBt.H₂O: amine or its salt (1 equivalent), acid (1 equivalent), EDAC.HCl (1 to 2 equivalents), HOBt.H₂O (1 to 2 equivalents) and base (1 to 3 equivalents). Compounds of formula FVII can also be prepared from acid chlorides FVIb and amines FV in the presence of a base such as triethylamine, diisopropylethylamine or pyridine in an aprotic solvent such as THF, benzene or halogenated hydrocarbons at temperatures from 20° C. to 90° C. for 2 to 24 hours. Compounds FVIII can be prepared from the corresponding protected amine FVII by removal of the protecting group (Pg). The methods for introducing and removing these protecting groups are known in the art (T. W. Green, Protective Groups in Organic Synthesis, John Wiley and Sons, Inc., 1981). Examples of protecting groups include benzyloxycarbonyl or t-butoxycarbonyl group and the like, but preferably t-butoxycarbonyl in which case the removal is carried in acidic conditions such as TFA or 4N HCl in dioxane at room temperature for 10 minutes to 24 hours. In these conditions, the amine FVIII is isolated as its hydrochloric or trifluoroacetic salt. Compounds If-II can be prepared in the conditions commonly used to form amide bonds, from the amine FVIII or its salts and acids FIX. Acids FIX can be prepared from the corresponding esters FII by using a base such as lithium hydroxide, sodium hydroxide, alkali carbonates (potassium or cesium) in a polar protic solvent such as methanol, ethanol, water or in mixtures of solvents including the mentioned polar protic solvent or other aprotic solvents. Typically, the hydrolysis is performed in an alcohol (methanol or ethanol) or in a 1:1 mixture of alcohol/THF, with water in the presence of sodium hydroxide (1-10 equivalents) at a temperature ranging from 20° C. to 100° C. for 0.5 to 24 hours. Acids FIX can also be prepared from the corresponding esters FII by acid hydrolysis using an acid such as TFA, HCl, H₂SO₄, AcOH or in a mixture of these acids in neat or aqueous condition at a temperature ranging from 20° C. to 100° C. for 0.5 to 24 hours. In the case wherein Rf¹² is benzyl group, acids FIX can be prepared from FII by hydrogenolysis using catalysts such as palladium on carbon or palladium hydroxide in a protic solvent such as EtOH or aprotic solvent such as EtOAc under hydrogen atmosphere at a pressure of 15 to 150 psi, at a temperature of 20° C. to 100° C. for 1 to 48 hours. Additional conditions for the hydrolysis of ester groups can be found in T. W. Green, Protective Groups in Organic Synthesis, John Wiley and Sons, Inc., 1981.

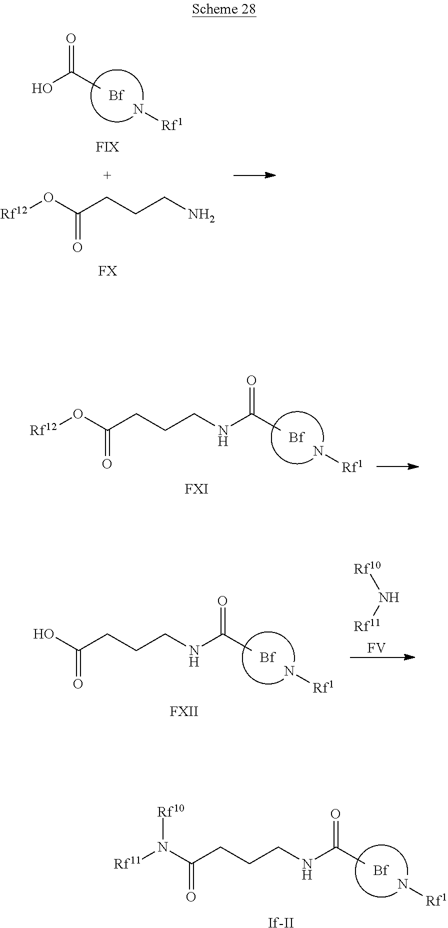

wherein the symbols are as defined above.

Alternatively, compounds If-II can be prepared according to the sequence described in Scheme 28. Acids FIX (obtained from esters FII as described above) can be coupled to an amine FX under conditions commonly employed to form amide bonds, to obtain the corresponding amide FXI. The ester group of the amide FXI can be deprotected (hydrolyzed in the preferred case where Rf¹² is an alkyl group such as methyl or ethyl group) under various conditions known in the art. Compounds If-II can be obtained by coupling the resulting acids FXII with amines FV or its salts following the conditions commonly employed to formamide bonds.

The conditions (solvent, reaction temperature, reaction time, chemical equivalent ratio) for each reaction in each of the above-mentioned production methods can be appropriately determined depending on the compound to be produced, the kind of reaction and the like.

In the thus-obtained compound of the present invention, the functional group in a molecule can also be converted to an objective functional group by combining chemical reactions known per se. As such chemical reactions, oxidation reaction, reduction reaction, alkylation reaction, hydrolysis, amination reaction, esterification reaction, aryl coupling reaction, deprotection and the like can be mentioned.

In the above-mentioned production method, when the starting compound has an amino group, a carboxyl group, a hydroxy group or a carbonyl group as a substituent, a protecting group generally used in peptide chemistry and the like may be introduced into these groups. By eliminating the protecting group as necessary after the reaction, the objective compound can be obtained.

As the amino-protecting group, for example, formyl group, C₁₋₆ alkyl-carbonyl group, C₁₋₆ alkoxy-carbonyl group, benzoyl group, C₇₋₁₀ aralkyl-carbonyl group (e.g., benzylcarbonyl), C₇₋₁₄ aralkyloxy-carbonyl group (e.g., benzyloxycarbonyl, 9-fluorenylmethoxycarbonyl), trityl group, phthaloyl group, N,N-dimethylaminomethylene group, substituted silyl group (e.g., trimethylsilyl, triethylsilyl, dimethylphenylsilyl, tert-butyldimethylsilyl, tert-butyldiethylsilyl), C₂₋₆ alkenyl group (e.g., 1-allyl) and the like can be mentioned. These groups are optionally substituted by 1 to 3 substituents selected from halogen atom, C₁₋₆ alkoxy group and nitro group.

As the carboxyl-protecting group, for example, C₁₋₆ alkyl group, C₇₋₁₁ aralkyl group (e.g., benzyl), phenyl group, trityl group, substituted silyl group (e.g., trimethylsilyl, triethylsilyl, dimethylphenylsilyl, tert-butyldimethylsilyl, tert-butyldiethylsilyl), C₂₋₆ alkenyl group (e.g., 1-allyl) and the like can be mentioned. These groups are optionally substituted by 1 to 3 substituents selected from halogen atom, C₁₋₆ alkoxy group and nitro group.

As the hydroxy-protecting group, for example, C₁₋₆ alkyl group, phenyl group, trityl group, C₇₋₁₀ aralkyl group (e.g., benzyl), formyl group, C₁₋₆ alkyl-carbonyl group, benzoyl group, C₇₋₁₀ aralkyl-carbonyl group (e.g., benzylcarbonyl), 2-tetrahydropyranyl group, 2-tetrahydrofuranyl group, substituted silyl group (e.g., trimethylsilyl, triethylsilyl, dimethylphenylsilyl, tert-butyldimethylsilyl, tert-butyldiethylsilyl), C₂₋₆ alkenyl group (e.g., 1-allyl) and the like can be mentioned. These groups are optionally substituted by 1 to 3 substituents selected from halogen atom, C₁₋₆ alkyl group, C₁₋₆ alkoxy group and nitro group.

As the carbonyl-protecting group, for example, cyclic acetal (e.g., 1,3-dioxane), non-cyclic acetal (e.g., di-C₁₋₆ alkylacetal) and the like can be mentioned.

For elimination of the above-mentioned protecting group, a method known per se, for example, a method described in Protective Groups in Organic Synthesis, John Wiley and Sons (1980) and the like can be mentioned. For example, employed is a method using acid, base, UV light, hydrazine, phenyl hydrazine, sodium N-methyldithiocarbamate, tetrabutylammonium fluoride, palladium acetate, trialkylsilyl halide (e.g., trimethylsilyl iodide, trimethylsilyl bromide and the like) and the like, reduction and the like.

In the above-mentioned production methods, the starting compound may be in the form of a salt. As such salt, those similar to the salts of the aforementioned compound of the present invention can be mentioned.

When the compound of the present invention contains an optical isomer, a stereoisomer, a positional isomer or a rotational isomer, these can be obtained as a single product according to a synthetic method and separation method known per se.

The compound of the present invention may be in the form of a crystal.

The crystal of the compound of the present invention can be produced by crystallization of the compound of the present invention according to a crystallization method known per se.

The crystal of the compound of the present invention is superior in physicochemical properties (e.g., melting point, solubility, stability and the like) and biological properties (e.g., pharmacokinetics (absorption, distribution, metabolism, excretion), efficacy expression and the like), and is extremely useful as a pharmaceutical agent.

### EXAMPLES

The present invention is explained in more detail by referring to the following Examples, Formulation Examples and Experimental Example, which are not to be construed as limitative.

In the examples described below, unless otherwise indicated all temperatures are set forth in degrees Celsius and ambient temperature, or room temperature, is typically 18° C. to 25° C.

Starting materials, the preparation of which are not described, are commercially available or can be readily prepared by known techniques from commercially available starting materials.

Reagents were purchased from commercial suppliers such as Aldrich Chemical Company, Lancaster, Acros international, TCI or Maybridge, and were used without further purification unless otherwise indicated.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe.

Yields are given for illustration only and are not necessarily the maximum attainable.

The intermediates and final products described herein may be isolated and purified by the conventional techniques known to artisans of organic chemistry. These techniques include, but are not limited to, concentration, concentration under reduced pressure, extraction with solvents, crystallization, recrystallization, transfer dissolution and chromatography. Chromatography was performed using glass column and silica gel 60 (230-400 mesh ASTM from EMD) or using medium pressure liquid chromatography (MPLC) Biotage systems (Flash+™ or Horizonrm HPFCT™, manufacturer: Dyax Corporation) using normal phase silica Flash+™ cartridges or reversed phase C18 Flash+™ cartridges. Reversed phase high pressure liquid chromatography (HPLC) was performed on a Parallex Flex™ Biotage system using an Xterra® prep RP18 OBD 10 μM 19×250 mm column from Waters. ¹H NMR spectra were recorded on a Varian instrument operating at 400 MHz. ¹H NMR spectra were obtained as CDCl₃ or DMSO-d₆ solutions (reported in ppm), using chloroform (7.25 ppm) or tetramethylsilane (0.00 ppm) as the reference standards. When peak multiplicities are reported, the following abbreviations are used: s (singlet), d (doublet), t (triplet), m (multiplet), br (broadened), dd (doublet of doublets), dt (doublet of triplets), tt (triplet of triplets). Coupling constants, when given, are reported in Hertz (Hz). LCMS were recorded on a Finnigan LCQduo from Thermoquest equipped with an Agilent Zorbax C18 rapid resolution 4.6×50 mm, 3.5 μm 80 Å column with APCI ionization or on a Surveyor MSQ from ThermoFinnigan direct injection with ESI ionization.

### 1-Phenyl-3-(trifluoromethyl)-1H-pyrazole-4-carboxylic acid

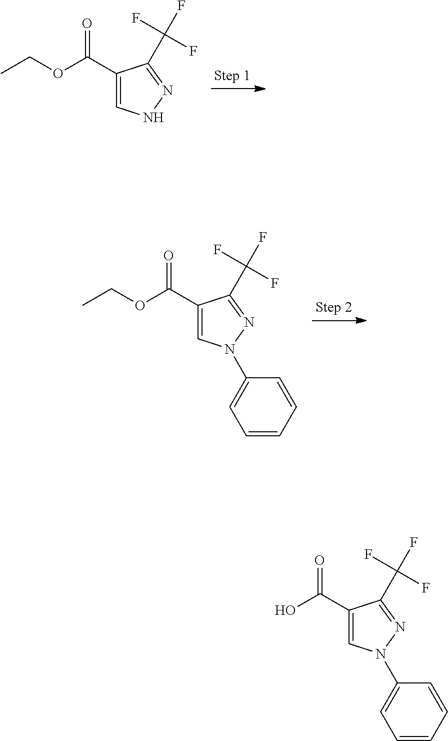

### Step 1

According to the procedure of Buchwald et al. (J. Org. Chem. 2004, 69, 5578), to a 350 mL sealed tube flushed vigorously with nitrogen was added ethyl 3-(trifluoromethyl)-1H-pyrazole-4-carboxylate (20.0 g, 96.1 mmol), 1-iodobenzene (12.9 mL, 115 mmol), potassium carbonate (27.9 g, 202 mmol), copper(I) iodide (0.915 g, 4.80 mmol), and (1S,2S)—N1,N2-dimethylcyclohexane-1,2-diamine (1.37 g, 9.61 mmol), followed by degassed (argon) toluene (100 mL). The mixture was stirred for 24 hours at 110° C., cooled to room temperature, and filtered through a short silica pad which was thoroughly rinsed with toluene and AcOEt. The filtrate was concentrated in vacuo to give ethyl 1-phenyl-3-(trifluoromethyl)-1H-pyrazole-4-carboxylate (21 g, 77%) as a solid: ¹H NMR (400 MHz, CDCl₃) δ 1.39 (t, 3H, J=7.0 Hz), 4.37 (q, 2H, J=7.0 Hz), 7.42 (m, 1H), 7.52 (m, 2H), 7.72 (m, 2H), 8.48 (m, 1H).

### Step 2

To a solution of ethyl 1-phenyl-3-(trifluoromethyl)-1H-pyrazole-4-carboxylate (0.800 g, 2.81 mmol) in EtOH (20 mL) and THF (5 mL) was added 2M aqueous sodium hydroxide (1.69 mL, 8.44 mmol). The mixture was heated to reflux until TLC indicated completion, cooled to room temperature, and concentrated in vacuo. The residue was dissolved in water and washed with ether. The aqueous layer was then adjusted to pH-4 with 10% HCl. The solid was collected by filtration and dried under vacuum to give 1-phenyl-3-(trifluoromethyl)-1H-pyrazole-4-carboxylic acid (0.560 g, 78%) as a white powder: ¹H NMR (400 MHz, DMSO-d₆) δ 7.47 (m, 1H), 7.58 (m, 2H), 7.94 (m, 2H), 9.24 (bs, 1H); m/z (APCI neg) 254.9 (100%)[M−H].

### N-(2-Aminoethyl)-1-phenyl-3-(trifluoromethyl)-1H-pyrazole-4-carboxamide

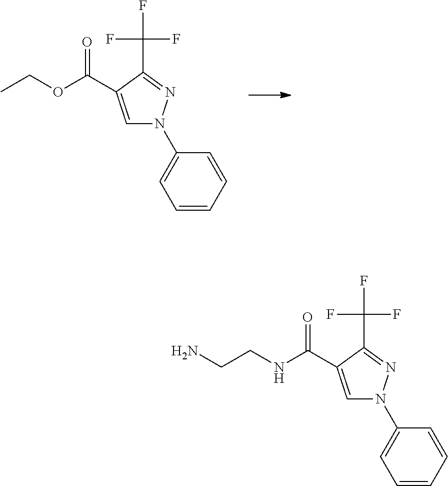

To ethyl 1-phenyl-3-(trifluoromethyl)-1H-pyrazole-4-carboxylate (20.0 g, 70.36 mmol) was added ethylenediamine (188.2 mL, 2815 mmol). The mixture was heated to reflux for 5 hours, cooled to room temperature, and concentrated in vacuo, and the residue was azeotroped several times with toluene to give N-(2-aminoethyl)-1-phenyl-3-(trifluoromethyl)-1H-pyrazole-4-carboxamide (20.84 g, 99%) as a tan solid: ¹H NMR (400 MHz, DMSO-d₆) δ 1.77 (bs, 2H), 2.68 (t, 2H, J=6.5 Hz), 3.23 (q, 2H, J=6.2 Hz), 7.47 (m, 1H), 7.60 (m, 2H), 7.83 (m, 2H), 8.28 (m, 1H), 9.11 (m, 1H); m/z (APCI pos) 299.0 (100%) [M+H].

### 4-Phenoxypiperidin-3-ol hydrochloride and 4-phenylpiperidin-3-ol hydrochloride

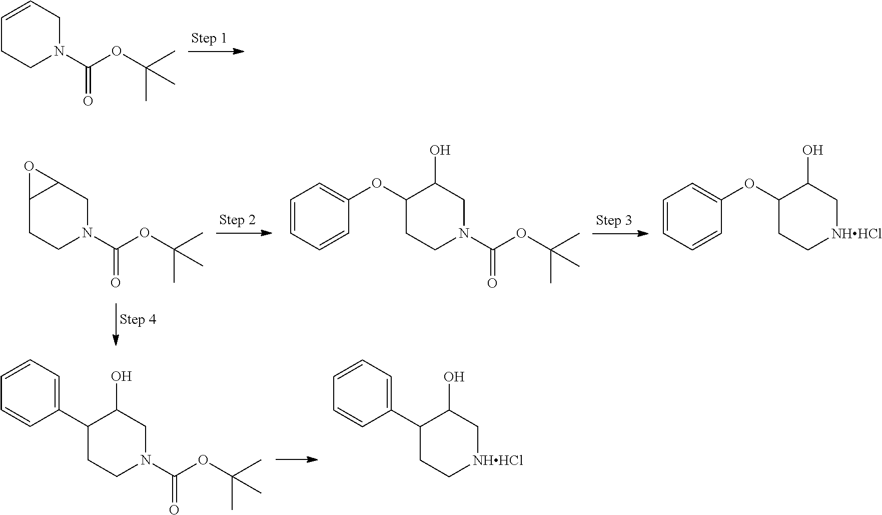

### Step 1

According to WO 97/10212, to a solution of tert-butyl 5,6-dihydropyridine-1(2H)-carboxylate (2.00 g, 10.9 mmol) in DCM was added MCPBA (4.16 g, 18.6 mmol) and the mixture was stirred for 16 hours. It was then washed with saturated aqueous sodium metabisulfite, saturated aqueous sodium bicarbonate, 1N NaOH, and brine. The organic layer was dried (sodium sulfate), filtered, and concentrated in vacuo to give tert-butyl 7-oxa-3-aza-bicyclo[4.1.0]heptane-3-carboxylate (2.04 g, 94%) as an oil: ¹H NMR (400 MHz, CDCl₃) δ 1.45 (s, 9H), 1.91 (m, 1H), 2.04 (br, 1H), 3.11 (m, 1H), 3.21 (br, 1H), 3.29 (m, 1H), 3.45 (br, 1H), 3.70 (br, 1H), 3.88 (br, 1H).

### Step 2

To a solution of tert-butyl 7-oxa-3-aza-bicyclo[4.1.0]heptane-3-carboxylate (3.44 g, 17.3 mmol) in EtOH was added K₂CO₃ (2.39 g, 17.3 mmol) and phenol (1.62 g, 17.3 mmol). The mixture was heated to reflux for 16 hours. The solvent was removed in vacuo and the residue was partitioned between AcOEt and water. The organic layer was dried (sodium sulfate), filtered, and concentrated in vacuo, and the residue was purified by chromatography on silica (15-25% AcOEt/hexanes) to give tert-butyl 3-hydroxy-4-phenoxypiperidine-1-carboxylate (1.5 g, 29%) as a oil that crystallized upon standing: ¹H NMR (400 MHz, DMSO-d₆) δ 1.46 (s, 9H), 1.61 (m, 1H), 1.91 (m, 1H), 2.41 (d, 1H, J=3.5 Hz), 3.10 (br, 2H), 3.82 (br, 2H), 4.06 (m, 1H), 4.21 (br, 1H), 6.96 (m, 3H), 7.29 (m, 2H).

### Step 4

According to Tetrahedron Lett., 1979, 17, 1503, to a stirred solution of phenylmagnesium chloride (0.753 mL, 1.51 mmol) and copper(I) iodide (0.0191 g, 0.100 mmol) in THF (3 mL) at −30° C. was added tert-butyl 7-oxa-3-aza-(0.200 g, 1.00 mmol) in THF (1 mL). The mixture was allowed to warm to room temperature and was stirred for 16 h. It was then quenched with saturated aqueous ammonium chloride and the mixture was extracted with AcOEt. The combined organic layers were dried (sodium sulfate), filtered, and concentrated in vacuo to give tert-butyl 3-hydroxy-4-phenylpiperidine-1-carboxylate (0.261 g, 94%) as an oil: ¹H NMR (400 MHz, DMSO-d₆) δ 1.49 (s, 9H), 1.76 (m, 3H), 2.50-2.78 (m, 3H), 3.70 (m, 1H), 4.30 (bd, 1H), 7.30 (m, 5H).

### Step 3

To a solution of tert-butyl 3-hydroxy-4-phenoxypiperidine-1-carboxylate (70 mg, 0.24 mmol) in DCM was added HCl (1 mL, a 4N solution in dioxane). The mixture was stirred for 2 hours then diluted with ether, and the precipitate was collected by filtration to give 4-phenoxypiperidin-3-ol hydrochloride (56 mg, 0.24 mmol, 100%) as a solid: m/z (APCI pos) 194.1 (100%) [M+H].

Compound of the following structure was prepared from tert-butyl 3-hydroxy-4-phenylpiperidine-1-carboxylate using a similar method to that described above.

| Structure | Name | Physical Data |
|---|---|---|
| 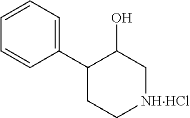 | 4- phenylpiperidin- 3-ol hydrochloride | Solid (100%); ¹H NMR (400 MHz, DMSO-d₆) δ 1.88 (m, 2H), 2.66 (m, 2H), 2.92 (m, 1H), 3.29 (m, 2H), 3.91 (m, 1H), 7.27 (m, 5H), 9.09 (m, 2H); m/z (APCI pos) 194.1 (100%) [M + H]. |

### 3-Ethoxy-4-phenoxypiperidine trifluoroacetate

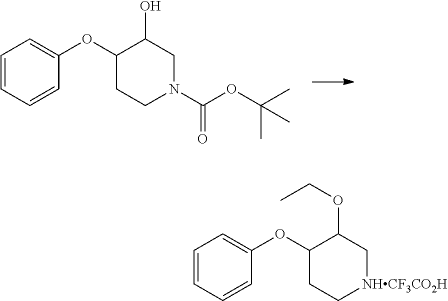

To a solution of tert-butyl 3-hydroxy-4-phenoxypiperidine-1-carboxylate (0.300 g, 1.02 mmol) in DMF was added NaH (49 mg, 1.23 mmol). After the gas evolution ceased, iodoethane (250 μl, 3.07 mmol) was added. The mixture was heated to 80° C. for 3 hours, cooled to room temperature and poured into iced water. The mixture was extracted with AcOEt, and the combined organic layers were dried (sodium sulfate), filtered, and concentrated in vacuo. The crude product was treated with TFA for 1 hour, concentrated in vacuo and dried to give 3-ethoxy-4-phenoxypiperidine trifluoroacetate (0.267 g, 78%) as an oil: ¹H NMR (400 MHz, DMSO-d₆) δ 1.13 (t, 3H, J=7.0 Hz), 1.81 (m, 1H), 2.12 (m, 1H), 3.09 (m, 3H), 3.30 (m, 1H), 3.64 (m, 3H), 4.57 (m, 1H), 6.98 (m, 1H), 7.04 (m, 2H), 7.31 (m, 2H), 8.56 (br, 1H), 8.79 (br, 1H).

### 4-(Propylsulfonyl)piperidine hydrochloride

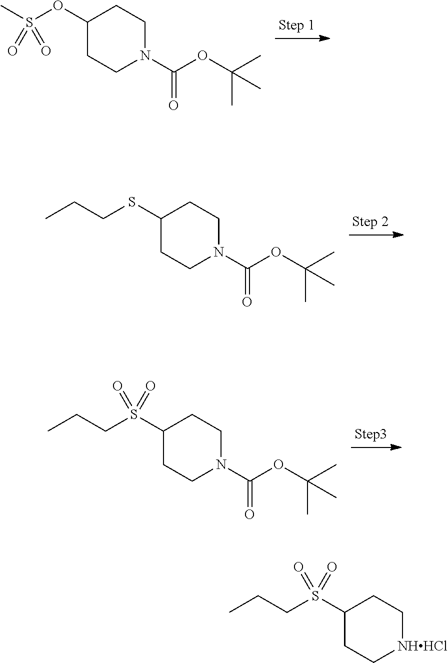

### Step 1

To tert-butyl 4-(methylsulfonyloxy)piperidine-1-carboxylate (1 g, 4 mmol) in DMF (100 mL) was added sodium propane-1-thiolate (0.53 g, 5.4 mmol) and the mixture was stirred at room temperature for 3 days. Water (200 mL) was added and the aqueous layer was extracted with DCM. The combined organic layers were washed with brine then dried over MgSO₄. Concentration yielded tert-butyl 4-(propylthio)piperidine-1-carboxylate as a tan oil (560 mg, 54%): ¹H NMR (400 MHz, CDCl₃) δ 0.99 (t, 3H, J=7.4 Hz), 1.46 (s, 9H), 1.56-1.65 (m, 2H), 1.80-1.95 (m, 2H), 2.50-2.55 (m, 2H), 2.74-2.80 (m, 1H), 2.85-2.95 (m, 2H), 3.80-4.02 (m, 4H).

### Step 2

To tert-butyl 4-(propylthio)piperidine-1-carboxylate (1.7 g, 6.6 mmol) in acetone (200 mL) was added slowly (exothermic reaction) potassium permanganate (2.6 g, 16 mmol). The mixture was stirred for 18 hours at room temperature then filtered through a plug of celite. The filtrate was concentrated to yield tert-butyl 4-(propylsulfonyl)piperidine-1-carboxylate as a clear oil (1.9 g, 80%): ¹H NMR (400 MHz, CDCl) δ 0.99 (t, 3H, J=7.4 Hz), 1.46 (s, 9H), 1.56-1.65 (m, 2H), 1.85-1.95 (m, 4H), 2.05-2.10 (m, 2H), 2.50-2.55 (m, 2H), 2.70-2.81 (m, 3H).

### Step 3

To tert-butyl 4-(propylsulfonyl)piperidine-1-carboxylate (1.5 g, 5.1 mmol) was added 4N HCl in dioxane (10 mL) and the mixture was concentrated to give 4-(propylsulfonyl)piperidine hydrochloride as a white solid (852 mg, 51%): ¹H NMR (400 MHz, DMSO-d₆) δ 1.00 (t, 3H, J=7.4 Hz), 1.60-1.90 (m, 4H), 2.10-2.20 (m, 2H), 2.85-2.98 (m, 3H), 3.08-3.12 (m, 2H), 3.35-3.42 (m, 2H).

### 4-(2,2,2-Trifluoroethoxy)piperidine acetate

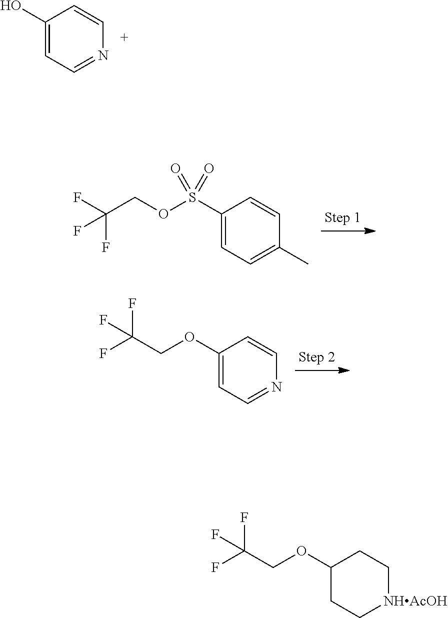

### Step 1

To a mixture of NaH (95%, 0.80 g, 31.5 mmol) and DMF (10 mL) was added dropwise a solution of pyridin-4-ol (3.0 g, 32 mmol) in DMF (20 mL) over a period of 20 min. The mixture was stirred for 1 hour at 40° C. under nitrogen atmosphere then, a solution of 2,2,2-trifluoroethyl p-toluenesulfonate (8.08 g, 31.5 mmol) in DMF (10 mL) was added and the mixture was stirred at 80° C. for 18 h. After cooling to room temperature, the mixture was diluted with AcOEt, washed with water and brine, dried and concentrated in vacuo. The residue was purified by silica gel column chromatography (hexanes/AcOEt 2/1->1/4) to give 4-(2,2,2-trifluoroethoxy)pyridine (0.34 g, 6%) as a pale yellow powder: ¹H NMR (400 MHz, CDCl₃) δ 4.42 (1H, d, J=8.0 Hz), 4.43 (1H, d, J=8.0 Hz), 6.87 (2H, dd, J=1.6, 4.8 Hz), 8.51 (2H, d, J=1.6, 4.8 Hz).

### Step 2

Under H₂ atmosphere, a suspension of 4-(2,2,2-trifluoroethoxy)pyridine (0.34 g, 1.92 mmol), PtO₂ (0.30 g), AcOH (5 mL) and EtOH (5 mL) was stirred for 18 hours at room temperature. The catalyst was removed by filtration and the filtrate was concentrated in vacuo to give 4-(2,2,2-trifluoroethoxy)piperidine acetate (0.24 g, 51%) as colorless crystals: ¹H NMR (400 MHz, CDCl₃) δ 1.68-1.80 (2H, m), 1.94-2.06 (5H, m), 2.82-2.92 (2H, m), 3.10-3.21 (2H, m), 3.68 (1H, m), 3.82 (1H, d, J=8.4 Hz), 3.86 (1H, d, J=8.4 Hz).

### 4-(1-Methyl-4-phenyl-1H-imidazol-5-yl)piperidine

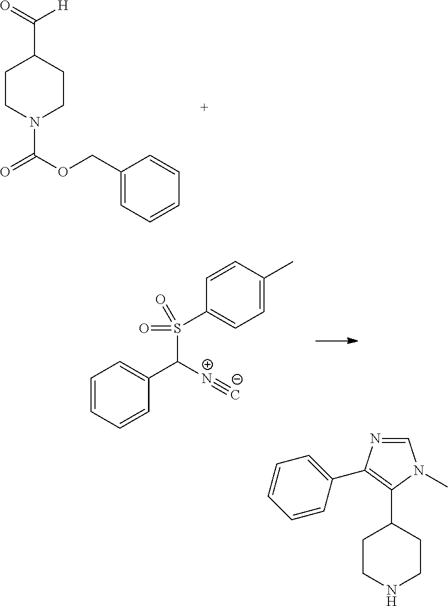

A solution of benzyl 4-formylpiperidine-1-carboxylate (7.4 g, 30 mmol) and methylamine (40 ml of a 1M solution in THF, 40 mmol) in THF was stirred at room temperature for 15 min, at which point 1-(isocyano(phenyl)methylsulfonyl)-4-methylbenzene (5.4 g, 20 mmol) and piperazine (2.2 g, 26 mmol) were added. The solution was stirred an additional 18 h and diluted with AcOEt and water, and the layers separated. The organic layer was stirred with 3 N HCl for 10 min and then made basic with solid K₂CO₃. The organic layer was concentrated then was dissolved in EtOH (100 mL), and 10% Pd/C was added. The reaction solution was purged with hydrogen, then placed under 1 atm of hydrogen. The mixture was stirred overnight at room temperature then the catalyst was filtered off through a pad of celite and the filtrate concentrated to yield 4-(1-methyl-4-phenyl-1H-imidazol-5-yl)piperidine as an off-white solid (3.7 g, 80%): ¹H NMR (400 MHz, CDCl₃) δ 1.70-1.78 (m, 2H), 1.85-1.98 (m, 2H), 2.62-2.70 (m, 2H), 3.00-3.10 (m, 1H), 3.13-3.19 (m, 2H), 3.74 (s, 3H), 7.25-7.31 (m, 1H), 7.36-7.41 (m, 3H), 7.46-7.50 (m, 2H); m/z (APCI pos) 242.3 [M+H].

### (3-Methylpiperidin-4-yl)(pyrrolidin-1-yl)methanone hydrochloride

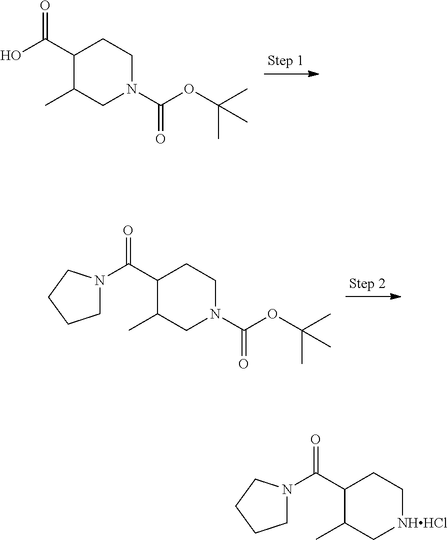

### Step 1

To 1-(tert-butoxycarbonyl)-3-methylpiperidine-4-carboxylic acid (100 mg, 0.4 mmol) in DCM (5 mL) were successively added pyrrolidine (29 mg, 0.4 mmol), EDAC.HCl (102 mg, 0.53 mmol), HOBt.H₂O (78 mg, 0.58 mmol) and triethylamine (125 mg, 1.23 mmol). The mixture was stirred at room temperature for 18 hours then washed with 2N HCl, 10% aqueous K₂CO₃ and brine. The organic layer was dried over MgSO₄ then concentrated to give tert-butyl 3-methyl-4-(pyrrolidine-1-carbonyl) piperidine-1-carboxylate as a clear oil (110 mg, 90%): ¹H NMR (400 MHz, CDCl₃) δ 0.95 (d, 3H, J=7.0 Hz), 1.45 (s, 9H), 1.80-2.01 (m, 6H), 2.65-2.70 (m, 1H), 3.00-3.16 (m, 2H), 3.40-3.54 (m, 4H), 3.75-4.00 (m, 4H).

### Step 2

To tert-butyl 3-methyl-4-(pyrrolidine-1-carbonyl)piperidine-1-carboxylate (100 mg, 0.34 mmol) was added 4N HCl in dioxane (5 mL). The mixture was stirred for 5 minutes then concentrated under vacuum to give (3-methyl piperidin-4-yl)(pyrrolidin-1-yl)methanone hydrochloride as a white solid (80 mg, quant.): ¹H NMR (400 MHz, DMSO-d₆) δ 0.90 (t, 3H, J=7.0 Hz), 1.70-1.90 (m, 8H), 2.89-3.02 (m, 3H), 3.15-3.31 (m, 3H), 3.43-3.48 (m, 2H), 9.0 (br, 1H).

### Piperidin-4-yl(pyridin-2-yl)methanone hydrochloride

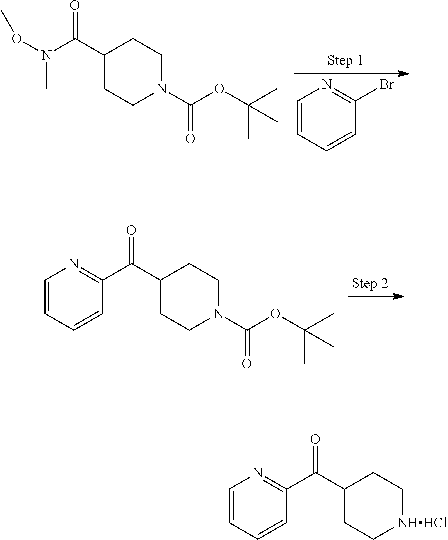

### Step 1

A solution of 2-bromopyridine (0.580 g, 3.67 mmol) in THF (10 mL) was added dropwise to a solution of n-butyllithium (3.01 mL, 7.53 mmol, 2.5 M in hexanes) in THF (10 mL) at −78° C. The mixture was stirred for 30 minutes then was treated with tert-butyl 4-(methoxy(methyl)carbamoyl)piperidine-1-carboxylate (1.00 g, 3.67 mmol) (prepared accordingly to US2002/0151717 or U.S. Pat. No. 6,465,490) as a solution in THF (3 mL). The material was allowed to warm to 0° C. over 15 min and was then quenched with a saturated aqueous NH₄Cl solution. The material was extracted with AcOEt, and the extract was dried and concentrated. Flash chromatography on silica gave tert-butyl 4-picolinoylpiperidine-1-carboxylate (0.542 g, 51%): ¹H NMR (400 MHz, CDCl₃) δ 1.47 (s, 9H), 1.65-1.69 (m, 2H), 1.84-1.93 (m, 2H), 2.92 (t, J=12.0 Hz, 2H), 3.99-4.06 (m, 1H), 4.11-4.17 (m, 2H), 7.45-7.49 (m, 1H), 7.85 (td, J=1.2, 7.6 Hz, 1H), 8.03 (d, J=6.8 Hz, 1H), 8.68 (dd, J=0.8, 4.0 Hz, 1H); m/z (APCI pos) 191.2 [M-Boc+H].

Compounds of the following structures were prepared from the corresponding aryl halides, using a similar method to that described above.

| Structure | Name | Physical Data |
|---|---|---|
| 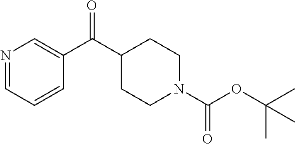 | tert-butyl 4- nicotinoyl- piperidine-1- carboxylate | ¹H NMR (400 MHz, CDCl₃) δ 1.47 (s, 9H), 1.67- 1.76 (m, 2H), 1.85- 1.88 (m, 2H), 2.92 (t, J = 12.0 Hz, 2H), 3.32- 3.41 (m, 1H), 4.11- 4.18 (m, 2H), 7.43- 7.46 (m, 1H), 8.22 (dt, J = 2.0, 8.4 Hz, 1H), 8.79 (dd, J = 1.6, 6.8 Hz, 1H), 9.16 (d, J = 1.6 Hz, 1H); m/z (APCI pos) 191.2 [M − Boc + H]. |
| 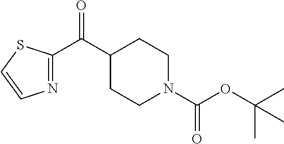 | tert-butyl 4- (thiazole-2- carbonyl) piperidine-1- carboxylate | ¹H NMR (400 MHz, CDCl₃) δ 1.47 (s, 9H), 1.69- 1.73 (m, 2H), 1.95- 1.98 (m, 2H), 2.92 (t, J = 12.0 Hz, 2H), 3.71- 3.79 (m, 1H), 4.11- 4.18 (m, 2H), 7.69 (d, J = 2.4 Hz, 1H), 8.01 (d, J = 3.2 Hz, 1H); m/z (APCI pos) 197.1 [M − Boc + H]. |

### Step 2

To tert-Butyl 4-picolinoylpiperidine-1-carboxylate (0.542 g, 1.87 mmol) was added HCl (9.33 mL, 37.3 mmol, 4M in dioxane). The solution was stirred at room temperature for 1 hour. The material was concentrated to give piperidin-4-yl(pyridin-2-yl)methanone hydrochloride (0.46 g, 94%): m/z (APCI pos) 191.1 [M+H].

Piperidin-4-yl(pyridin-3-yl)methanone hydrochloride and piperidin-4-yl(thiazol-2-yl)methanone hydrochloride were obtained from the corresponding N-Boc analogs by a similar method described above. These compounds were used for following reactions without analyzing chemical properties.

### (1-Methyl-1H-pyrazol-4-yl) (piperidin-4-yl)methanone hydrochloride

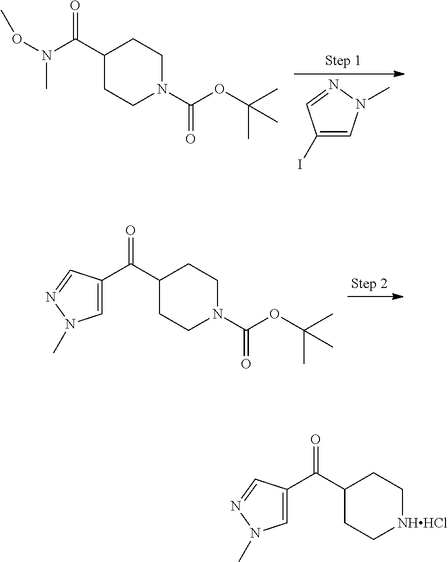

### Step 1

A solution of 4-iodo-1-methyl-1H-pyrazole (0.764 g, 3.67 mmol) in THF (10 mL) was slowly added to a solution of n-butyllithium (3.01 mL, 7.53 mmol, 2.5M in hexanes) in THF (10 mL) at −40° C. The mixture was stirred for 30 minutes, then cooled to −78° C. and tert-butyl 4-(methoxy(methyl)carbamoyl)piperidine-1-carboxylate (1.00 g, 3.67 mmol) as a solution in THF (3 mL) was added. The mixture was allowed to warm to 0° C. over 15 minutes and was quenched with a saturated aqueous NH₄Cl solution. The solution was extracted with AcOEt, and the extract was dried, and concentrated. Flash chromatography on silica gave tert-butyl 4-(1-methyl-1H-pyrazole-4-carbonyl)piperidine-1-carboxylate (0.315 g, 29%): ¹H NMR (400 MHz, CDCl₃) δ 1.46 (s, 9H), 1.66-1.90 (m, 4H), 2.86 (t, J=12.0 Hz, 2H), 2.93-3.02 (m, 1H), 3.94 (s, 3H), 4.10-4.22 (m, 2H), 7.89 (d, J=2.4 Hz, 1H); m/z (APCI pos) 194.2 [M-Boc+H].

The compound of the following structure was prepared from the corresponding aryl halide using a similar method to that described above.

| Structure | Name | Physical Data |
|---|---|---|
| 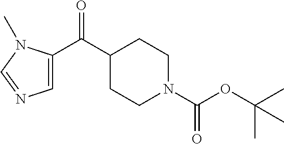 | tert-butyl 4- (1-methyl-1H- imidazole-5- carbonyl) piperidine-1- carboxylate | m/z (APCI pos) 194.1 [M − Boc + H]. |

### Step 2

To tert-Butyl 4-(1-methyl-1H-pyrazole-4-carbonyl)piperidine-1-carboxylate (0.315 g, 1.07 mmol) was added HCl (5.37 mL, 21.5 mmol, 4M in dioxane). The solution was stirred at room temperature for 1 hour then concentrated to provide (1-methyl-1H-pyrazol-4-yl)(piperidin-4-yl)methanone hydrochloride (0.236 g, 96%): m/z (APCI pos) 194.2 [M+H].

(1-Methyl-1H-imidazol-5-yl)(piperidin-4-yl)methanone hydrochloride was obtained from the corresponding N-Boc analog by a similar method described above. This compound was used as is without analyzing chemical properties.

### Phenyl(piperidin-4-yl)methanone hydrochloride

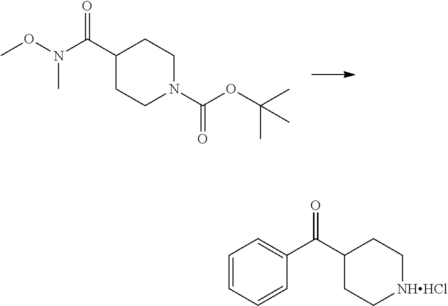

To a stirring solution of tert-butyl 4-(methoxy(methyl)carbamoyl)piperidine-1-carboxylate (0.133 g, 0.488 mmol) in THF (1 mL) at −40° C. was added phenylmagnesium chloride (0.269 mL, 0.537 mmol, 2M in THF) dropwise. The mixture was allowed to warm up to room temperature and was quenched with aqueous saturated ammonium chloride, then extracted with AcOEt (2×). The crude product was purified by chromatography on silica gel (20% AcOEt/hexanes) to give tert-butyl 4-benzoylpiperidine-1-carboxylate (0.030 g, 21%) as an oil: ¹H NMR (400 MHz, CDCl₃) δ 1.47 (s, 9H), 1.72 (m, 2H), 1.84 (m, 2H), 2.90 (m, 2H), 3.41 (m, 1H), 4.16 (br, 2H), 7.48 (m, 2H), 7.58 (m, 1H), 7.94 (m, 2H). To this oil was added HCl (2.59 mL, 10.4 mmol, 4M in dioxane) and the solution was stirred for 30 minutes at room temperature. The solvent was removed in vacuo to give phenyl(piperidin-4-yl)methanone hydrochloride (0.023 g, 98%) as a solid: ¹H NMR (400 MHz, DMSO-d₆) δ 1.75 (m, 2H), 1.95 (m, 2H), 3.04 (m, 2H), 3.48 (m, 2H), 3.69 (m, 2H), 3.77 (m, 1H), 7.56 (m, 2H), 7.68 (m, 1H), 8.00 (m, 2H).

### Phenyl(pyrrolidin-3-yl)methanone hydrochloride

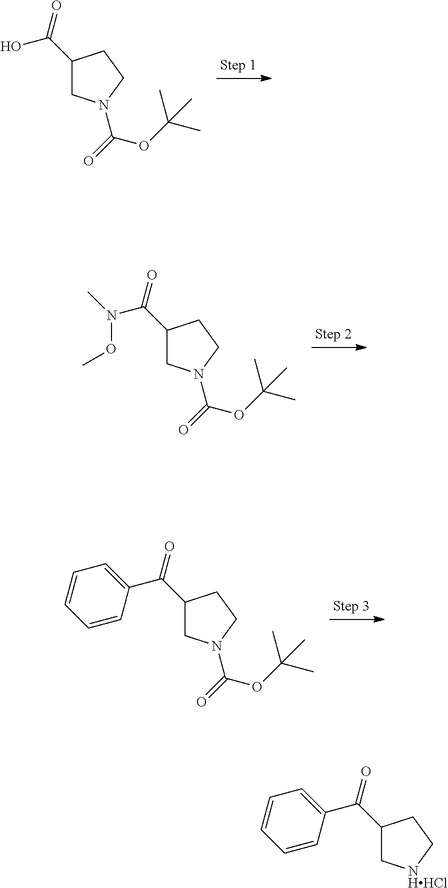

### Step 1

To a solution of 1-(tert-butoxycarbonyl)pyrrolidine-3-carboxylic acid (2.00 g, 9.29 mmol) in DCM (200 mL) was added 1,1′-carbonyldiimidazole (1.66 g, 10.2 mmol) portionwise. The mixture was stirred at room temperature for 2 hours then, N-methoxymethanamine hydrochloride (0.997 g, 10.2 mmol) was added in one portion. The mixture was stirred at room temperature for 18 hours then washed with 1N HCl (100 mL), sat. aqueous NaHCO₃ (100 mL) and brine and dried over Na₂SO₄. Concentration gave tert-butyl 3-(methoxy(methyl)carbamoyl)pyrrolidine-1-carboxylate (2.31 g, 96%) as a tan solid: m/z (APCI pos) 159 (100%) [M+H-Boc].

The compound of the following structure was prepared from 1-(tert-butoxycarbonyl)-4-isopropylpyrrolidine-3-carboxylic acid, using a similar method to that described above.

| Structure | Name | Physical Data |
|---|---|---|
| 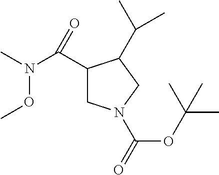 | tert-butyl 3- isopropyl-4- (methoxy (methyl) carbamoyl) pyrrolidine- 1- carboxylate | m/z (APCI pos) 201 (100%) [M + H − Boc]. |

### Step 2

A solution of tert-butyl 3-(methoxy(methyl)carbamoyl)pyrrolidine-1-carboxylate (1.00 g, 3.87 mmol) in THF (15 mL) was added dropwise to phenyllithium 1.8M (2.30 mL, 4.14 mmol) in THF (20 mL) at −78° C. The mixture was allowed to warm to room temperature overnight then it was cooled back to −78° C., and phenyllithium 1.8M (2.5 mL, 4.5 mmol) was added. The mixture was allowed to reach room temperature and was further stirred for 18 hours before being quenched for an hour at room temperature with sat. aqueous NH₄Cl (20 mL). The layers were separated and the aqueous layer was extracted with Et₂O. The combined organic layers were washed with brine, dried over Na₂SO₄ and concentrated to give a residue which was purified by chromatography on silica eluting with 25% AcOEt/hexanes to give tert-butyl 3-benzoylpyrrolidine-1-carboxylate (0.600 g, 56%) as a white solid: m/z (APCI pos) 176 (100%) [M+H-Boc].

The compound of the following structure was prepared from tert-butyl 3-isopropyl-4-(methoxy(methyl)carbamoyl)pyrrolidine-1-carboxylate, using a similar method to that described above.

| Structure | Name | Physical Data |
|---|---|---|
| 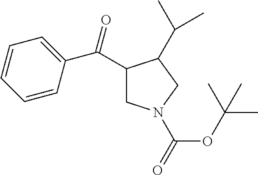 | tert-butyl 3- benzoyl-4- isopropyl- pyrrolidine-1- carboxylate | m/z (APCI pos) 218 (100%) [M + H − Boc]. |

### Step 3

To a solution of tert-butyl 3-benzoylpyrrolidine-1-carboxylate (0.600 g, 2.18 mmol) in DCM (40 mL) was added TFA (10 mL) and the mixture was stirred at room temperature for 1 hour. Concentration yielded a residue which was dissolved in MeOH (10 mL) and 1M HCl in Et₂O (100 ml) was added. The slurry was stirred for 1 hour at room temperature then concentrated to dryness to give phenyl(pyrrolidin-3-yl)methanone hydrochloride (0.435 g, 94%) as a tan solid: m/z (APCI pos) 176 (100%) [M+H].

The compound of the following structure was prepared from tert-butyl 3-benzoyl-4-isopropylpyrrolidine-1-carboxylate, using a similar method to that described above.

| Structure | Name | Physical Data |
|---|---|---|
| 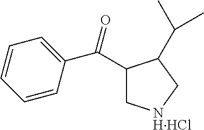 | (4- isopropyl- pyrrolidin-3- yl)(phenyl) methanone hydrochloride | m/z (APCI pos) 218 (100%) [M + H]. |

### 4,5-Dimethyl-2-(piperidin-4-yl)thiazole

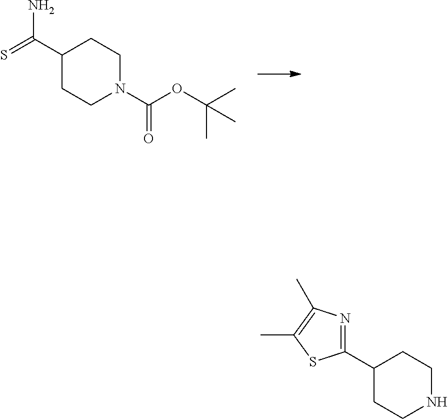

3-Bromobutan-2-one (124 mg, 0.8 mmol) and tert-butyl 4-carbamothioylpiperidine-1-carboxylate (200 mg, 0.8 mmol) in ethanol (1 were heated to 200° C. for 1 minute under microwave irradiation. The black residue was purified by chromatography (DCM/MeOH/NH₄OH 85/15/0.1) to yield 4,5-dimethyl-2-(piperidin-4-yl)thiazole as a brown solid (32 mg, 13%): ¹H NMR (400 MHz, DMSO-d₆) δ 1.76-1.88 (m, 2H), 2.08-2.14 (m, 2H), 2.21 (s, 3H), 2.30 (s, 3H), 2.97-3.05 (m, 2H), 3.18-3.24 (m, 1H), 3.30-3.35 (m, 2H); m/z (APCI pos) 197.1 (100%) [M+H].

### 2-Phenyl-4,5,6,7-tetrahydrothiazolo[5,4-c]pyridine hydrobromide

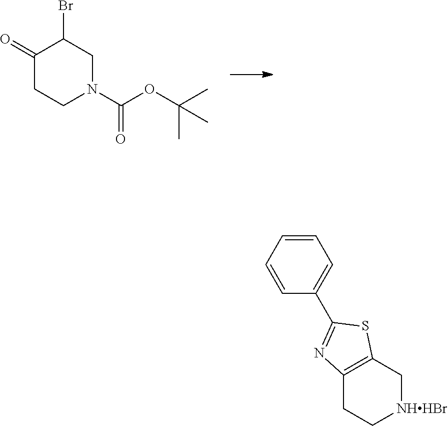

tert-Butyl 3-bromo-4-oxopiperidine-1-carboxylate (1.0 g, 3.6 mmol) and benzothioamide (490 mg, 3.6 mmol) were heated neat at 140° C. for 10 minutes. After cooling down, the resulting brown solid was sonicated in AcOEt for 10 minutes. Filtration and drying yielded 2-phenyl-4,5,6,7-tetrahydrothiazolo[5,4-c]pyridine hydrobromide as a brown solid (829 mg, 78%): ¹H NMR (400 MHz, DMSO-d₆) δ 3.06-3.12 (m, 2H), 3.49-3.57 (m, 2H), 4.46-4.53 (m, 2H), 7.50-7.56 (m, 3H), 7.90-7.94 (m, 2H), 9.35 (br, 2H); m/z (APCI pos) 217.1 (10%) [M+H].

The compound of the following structure was prepared from the corresponding thioamide, using a similar method to that described above.

| Structure | Name | Physical Data |
|---|---|---|
| 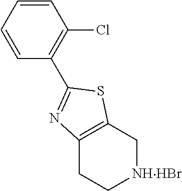 | 2-(2- chlorophenyl)- 4,5,6,7- tetrahydrothiazolo [5,4-c] pyridine hydrobromide | Beige solid (61%); ¹H NMR (400 MHz, DMSO-d₆) δ 3.08-3.15 (m, 2H), 3.52-3.59 (m, 2H), 4.51-4.56 (m, 2H), 7.48-7.56 (m, 2H), 7.64-7.69 (m, 1H), 8.12-8.16 (m, 1H), 9.27 (br, 2H); m/z (APCI pos) 251.0 (10%) [M + H]. |

### 2-Phenyl-5-(piperidin-4-yl)-1,3,4-oxadiazole hydrochloride

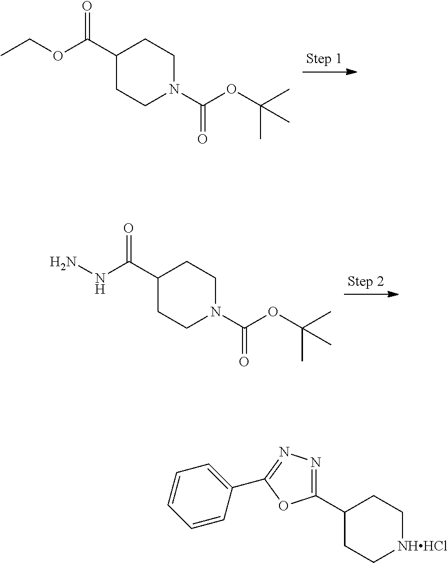

### Step 1

To 1-tert-butyl 4-ethyl piperidine-1,4-dicarboxylate (4.96 g, 19.3 mmol) in methanol (20 mL) was added hydrazine hydrate (5 mL) and the mixture was heated to reflux for 3 days. The mixture was concentrated to dryness to give tert-butyl 4-(hydrazinocarbonyl)piperidine-1-carboxylate as a white crystalline solid (4.2 g, 90%): ¹H NMR (400 MHz, DMSO-d₆) δ 1.39 (s, 9H), 1.39-1.42 (m, 2H), 1.55-1.64 (m, 2H), 2.18-2.27 (m, 1H), 2.63-2.76 (m, 2H), 3.89-3.97 (m, 2H), 9.03 (br, 1H); m/z (APCI pos) 144.1 (100%) [M+H-Boc].

The compound of the following structure was prepared from 1-tert-butyl 4-ethyl 4-methylpiperidine-1,4-dicarboxylate, using a similar method to that described above.

| Structure | Name | Physical Data |
|---|---|---|
| 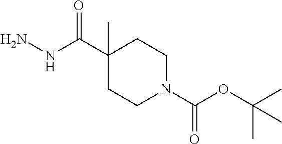 | tert-butyl 4- (hydrazinocarbonyl)- 4- methylpiperidine- 1-carboxylate | Clear paste (96%); ¹H NMR (400 Mhz, DMSO-d₆) δ 1.09 (s, 3H), 1.16-1.30 (m, 2H), 1.33 (s, 9H), 1.80-1.90 (m, 2H), 2.80-3.00 (m, 2H), 3.40-3.58 (m, 2H); m/z (APCI pos) 158.1 (40%) [M + H − Boc]. |

### Step 2

Ethyl benzimidate hydrochloride (954 mg, 5.1 mmol) was added to a solution of tert-butyl 4-(hydrazinocarbonyl)piperidine-1-carboxylate (500 mg, 2.0 mmol) in ethanol (10 mL). The mixture was heated to reflux for 20 hours then cooled down, and filtrated and the filtrate was concentrated. The residue was purified by chromatography (AcOEt/hexanes 30/70) to yield tert-butyl 4-(5-phenyl-1,3,4-oxadiazol-2-yl)piperidine-1-carboxylate as a white solid (437 mg, 65%). To this solid (400 mg, 1.2 mmol) was added 4N HCl in dioxane (10 mL) and the mixture was stirred at room temperature for 2 hours. After concentration, 2-phenyl-5-(piperidin-4-yl)-1,3,4-oxadiazole hydrochloride was isolated as a white solid (303 mg, 94%): ¹H NMR (400 MHz, DMSO-d₆) 2.00-2.10 (m, 2H), 2.22-2.30 (m, 2H), 3.02-3.12 (m, 2H), 3.28-3.36 (m, 2H), 3.40-3.48 (m, 1H), 7.58-7.65 (m, 3H), 7.98-8.01 (m, 2H), 9.04 (br, 1H), 9.22 (br, 1H); m/z (APCI pos) 230.1 (100%) [M+H].

The compound of the following structure was prepared from tert-butyl 4-(hydrazinocarbonyl)-4-methylpiperidine-1-carboxylate, using a similar method to that described above.

| Structure | Name | Physical Data |
|---|---|---|
| 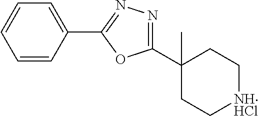 | 2-(4- methylpiperidin- 4-yl)-5- phenyl-1,3,4- oxadiazole hydrochloride | White solid (75%); ¹H NMR (400 MHz, DMSO-d₆) δ 1.44 (s, 3H), 1.97-2.12 (m, 2H), 2.33-2.41 (m, 2H), 2.97-3.07 (m, 2H), 3.20-3.27 (m, 2H), 7.58-7.67 (m, 3H), 8.01-8.04 (m, 2H), 9.20 (br, 1H), 9.30 (br, 1H); m/z (APCI pos) 244.1 (100%) [M + H − Boc]. |

### 5-Isopropyl-3-(piperidin-4-yl)-1,2,4-oxadiazole hydrochloride

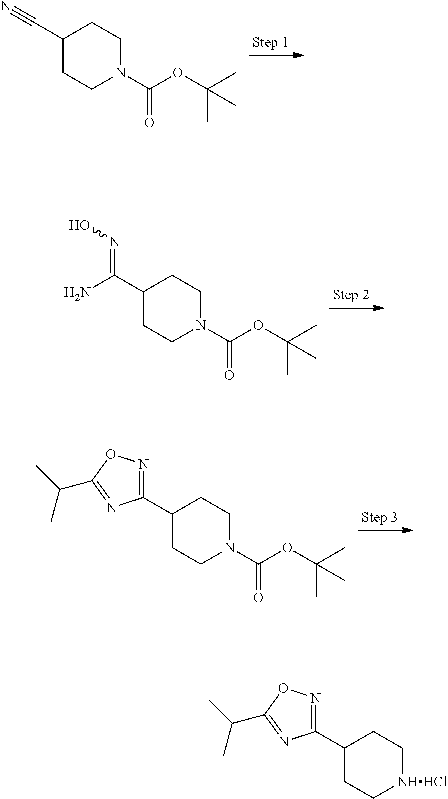

### Step 1

To tert-butyl 4-cyanopiperidine-1-carboxylate (1.5 g, 7.1 mmol) in EtOH (18 mL) and water (4 mL) were added hydroxylamine hydrochloride (1.5 g, 21 mmol) and potassium carbonate (1.6 g, 11 mmol). The mixture was stirred at room temperature for 1 hour then heated to reflux for 18 hours.

After cooling down, the mixture was filtered and the filtrate was concentrated. EtOH (50 mL) was added and the solution boiled for 5 minutes and filtered hot. The filtrate was concentrated to give a clear paste. AcOEt (100 mL) was added and the suspension sonicated for 5 minutes. Filtration yielded tert-butyl 4-(N′-hydroxycarbamimidoyl)piperidine-1-carboxylate as a white solid (1.6 g, 92%): ¹H NMR (400 MHz, CDCl₃) δ 1.37-1.49 (m, 4H), 1.39 (s, 9H), 1.63-1.70 (m, 4H), 2.09-2.18 (m, 1H), 3.90-3.98 (m, 2H), 8.80 (s, 1H).

### Step 2

To isobutyric acid (100 mg, 1.14 mmol) in DMF (5 ml) were added diisopropylethylamine (131 mg, 1.14 mmol) and tetramethylfluoroformamidinium hexafluorophosphate (300 mg, 1.14 mmol) at room temperature. The mixture was stirred at to room temperature for 30 minutes then tert-butyl 4-(N′-hydroxycarbamimidoyl)piperidine-1-carboxylate (276 mg, 1.14 mmol) was added at once. The mixture was stirred at 110° C. for 3 hours then concentrated to dryness. The residue was dissolved in AcOEt (50 mL) and washed with 1N HCl (2×50 mL) and brine. The organic layer was dried over MgSO₄ then concentrated to yield tert-butyl 4-(5-isopropyl-1,2,4-oxadiazol-3-yl)piperidine-1-carboxylate as a clear oil (331 mg, quant.): ¹H NMR (400 MHz, CDCl₃) δ 1.39 (d, 6H, J=7.0 Hz), 1.46 (s, 9H), 1.70-1.82 (m, 2H), 1.95-2.04 (m, 2H), 2.89-2.96 (m, 3H), 3.15-3.24 (m, 1H), 4.14-4.20 (m, 2H); m/z (APCI pos) 196.2 (M-Boc+H).

The compound of the following structure was prepared from the corresponding acid, using a similar method to that described above.

| Structure | Name | Physical Data |
|---|---|---|
| 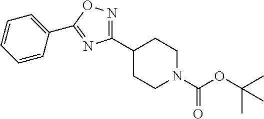 | tert-butyl 4- (5-phenyl- 1,2,4- oxadiazol-3- yl)piperidine- 1-carboxylate | Clear oil (quant.); ¹H NMR (400 MHz, CDCl₃) δ 1.48 (s, 9H), 1.79-1.90 (m, 2H), 2.03-2.10 (m, 2H), 2.90-3.07 (m, 3H), 4.09-4.20 (m, 2H), 7.45-7.61 (m, 3H), 8.10-8.14 (m, 2H); m/z (APCI pos) 230.1 (M − Boc + H) |

### Step 3

To tert-butyl 4-(5-isopropyl-1,2,4-oxadiazol-3-yl)piperidine-1-carboxylate (330 mg, 1.12 mmol) was added 4N HCl in dioxane (10 mL) and the mixture was stirred at room temperature for 1 hour. Concentration yielded 5-isopropyl-3-(piperidin-4-yl)-1,2,4-oxadiazole hydrochloride as a white solid (264 mg, quant.): ¹H NMR (400 MHz, CDCl₃) δ 1.31 (d, 6H, J=7.0 Hz), 1.85-1.96 (m, 2H), 2.05-2.14 (m, 2H), 2.95-3.06 (m, 2H), 3.10-3.20 (m, 1H), 3.24-3.30 (m, 2H); m/z (APCI pos) 296.1 [M+H].

The compound of the following structure was prepared from tert-butyl 4-(5-phenyl-1,2,4-oxadiazol-3-yl)piperidine-1-carboxylate, using a similar method to that described above.

| Structure | Name | Physical Data |
|---|---|---|
| 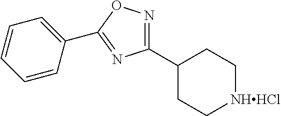 | 5-phenyl-3- (piperidin-4- yl)-1,2,4- oxadiazole hydrochloride | White solid (quant.); ¹H NMR (400 MHz, CDCl₃) δ 1.94-2.05 (m, 2H), 2.15-2.21 (m, 2H), 3.00-3.11 (m, 2H), 3.25-3.35 (m, 3H), 7.61-7.75 (m, 3H), 8.09-8.12 (m, 2H); m/z (APCI pos) 230.1 (M + H) |

### Ethyl 2-(2-methyl-1H-imidazol-4-yl)-4,5,6,7-tetrahydrobenzo[d]thiazole-6-carboxylate

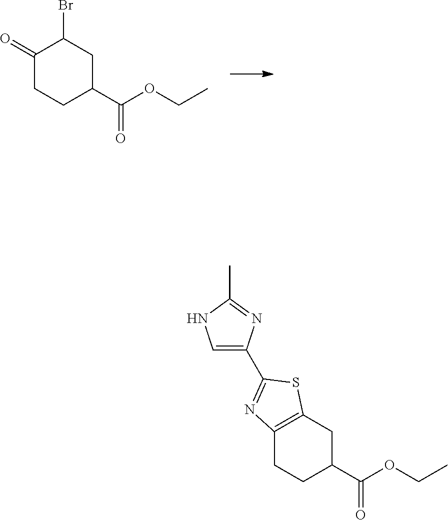

To ethyl 3-bromo-4-oxocyclohexanecarboxylate (1.00 g, 4.01 mmol) (prepared accordingly to Eur. J. Med. Chem.—Chim. Ther. 1984, 19(5), 457) in EtOH (5 mL) was added 2-methyl-1H-imidazole-4-carbothioamide (0.567 g, 4.01 mmol) and the mixture was heated to 120° C. for 15 minutes in an opened vessel. The residue was dried under vacuum and re-crystallized from EtOH/hexanes to provide ethyl 2-(2-methyl-1H-imidazol-4-yl)-4,5,6,7-tetrahydrobenzo[d]thiazole-6-carboxylate (0.500 g, 43%) as a yellow solid: ¹H NMR (400 MHz, DMSO-d₆) δ 1.21 (t, J=7.2 Hz, 3H), 1.90-1.98 (m, 1H), 2.16-2.24 (m 1H), 2.58 (s, 3H), 2.84 (t, J=6.0 Hz, 2H), 2.90-3.04 (m, 2H), 3.10-3.17 (m, 1H), 4.12 (q, J=6.4, 13.6 Hz, 2H), 8.19 (s, 1H); m/z (APCI pos) 292.2 [M+H].

Compounds of the following structures were prepared from the corresponding thioamides, using a similar method to that described above.

| Structure | Name | Physical Data |
|---|---|---|
| 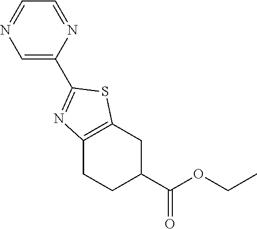 | ethyl 2- (pyrazin-2-yl)- 4,5,6,7- tetrahydrobenzo [d]thiazole-6- carboxylate | m/z (APCI pos) 290.2 [M + H] |
| 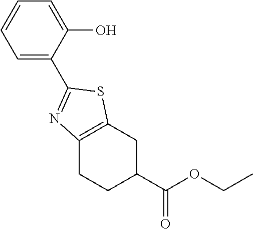 | ethyl 2-(2- hydroxyphenyl)- 4,5,6,7- tetrahydrobenzo [d]thiazole-6- carboxylate | m/z (APCI pos) 304.2 [M + H] |
| 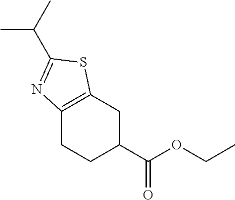 | ethyl 2- isopropyl- 4,5,6,7- tetrahydrobenzo [d]thiazole-6- carboxylate | m/z (APCI pos) 254.2 [M + H] |

### Ethyl 2-phenyl-4,5,6,7-tetrahydrobenzo[d]oxazole-6-carboxylate

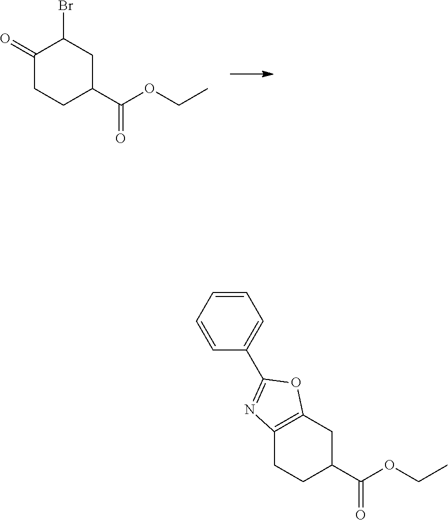

Ethyl 3-bromo-4-oxocyclohexanecarboxylate (1.00 g, 4.01 mmol) and benzamidine (0.482 g, 4.01 mmol) in 1,2-dichloroethane (3 mL) were stirred together in a sealed tube at 120° C. for 1 hour. The solution was cooled, concentrated and purified by flash chromatography to afford ethyl 2-phenyl-4,5,6,7-tetrahydrobenzo[d]oxazole-6-carboxylate (0.382 g, 35%); m/z (APCI pos) 272.2 [M+H].

### 2-tert-Butyl-5,6,7,8-tetrahydroimidazo[1,2-a]pyridine-6-carboxylic acid

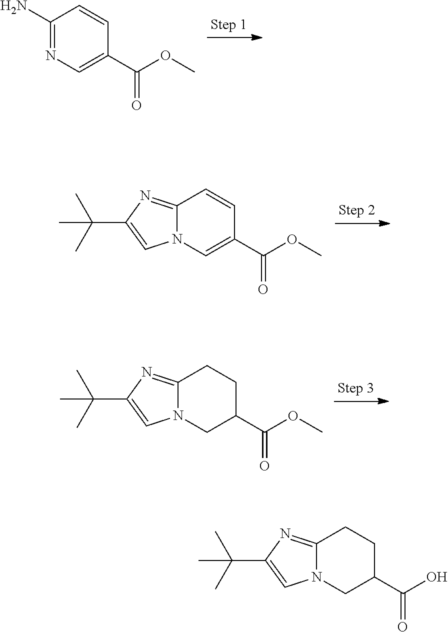

### Step 1

A mixture of methyl 6-aminonicotinate (1.50 g, 9.86 mmol), 1-bromo-3,3-dimethylbutan-2-one (1.77 g, 9.86 mmol), and NaHCO₃ (0.828 g, 9.86 mmol) in MeOH (30 mL) was heated at reflux for 18 hours. The mixture was cooled and concentrated to a residue. The residue was diluted with AcOEt, and the solution was washed with water and brine, dried over Na₂SO₄, and concentrated to a residue. This residue was crystallized from AcOEt/EtOH to afford methyl 2-tert-butylimidazo[1,2-a]pyridine-6-carboxylate (0.690 g, 30%) as a solid: m/e (APCI pos) 233 [M+H].

### Step 2

Methyl 2-tert-butylimidazo[1,2-a]pyridine-6-carboxylate (0.200 g, 0.861 mmol) was dissolved in EtOH (30 mL) and HCl (0.144 mL, 0.861 mmol) and the reaction pressure vessel was purged with nitrogen. Pd/C (0.367 g, 0.172 mmol) was added to the reaction pressure vessel and the vessel was purged with nitrogen three times. The vessel was then flushed with H₂ three times and pressurized to 250 psi. The reaction vessel was heated in an oil bath at 75° C. for 18 hours then cooled down, the catalyst was filtered and the filtrate concentrated to a residue which was purified by chromatography on silica (7% MeOH/DCM) to give methyl 2-tert-butyl-5,6,7,8-tetrahydroimidazo[1,2-a]pyridine-6-carboxylate (0.100 g, 49%) as a white solid: ¹H NMR (400 MHz, CDCl₃) δ 1.27 (s, 9H), 1.99-2.09 (m, 1H), 2.30-2.36 (m, 1H), 2.81-3.05 (m, 3H), 3.76 (s, 3H), 4.03-4.15 (m, 2H), 6.49 (s, 1H); m/z (APCI pos) 236 [M+11].

### Step 3

To methyl 2-tert-butyl-5,6,7,8-tetrahydroimidazo[1,2-a]pyridine-6-carboxylate (0.100 g, 0.423 mmol) in water (3 mL) was added concentrated HCl (0.0705 mL, 0.423 mmol) and the mixture was heated at reflux for 4 hours. The mixture was concentrated to dryness to give 2-tert-butyl-5,6,7,8-tetrahydroimidazo[1,2-a]pyridine-6-carboxylic acid (0.090 g, 96%): m/e (APCI neg) 236 [M−H].

### trans-Methyl 4-(5-phenyl-1,3,4-oxadiazol-2-yl)cyclohexanecarboxylate

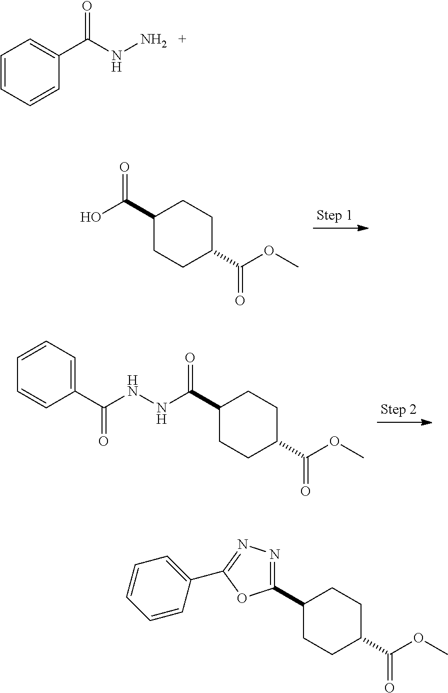

### Step 1

A solution of oxalyl chloride (0.26 mL, 2.95 mmol) in DCM (2 mL) was added dropwise over 20 min to a mixture of trans-4-(methoxycarbonyl)cyclohexanecarboxylic acid (0.50 g, 2.69 mmol), 2 drops of DMF and DCM (5 mL) at room temperature. After stirring for 30 min at room temperature, the mixture was concentrated in vacuo to dryness to give trans-methyl 4-(chlorocarbonyl)cyclohexanecarboxylate as a colorless oil. Then, benzohydrazide (0.41 g, 2.95 mmol) was added to a mixture of this oil, triethylamine (0.75 mL, 5.37 mmol) and DCM (10 mL), and the mixture was stirred for 18 h at room temperature. The precipitate was collected by filtration, washed with DCM and water and dried to give trans-methyl 4-[(2-benzoylhydrazino)carbonyl]cyclohexanecarboxylate (0.45 g) as a colorless powder. The filtrate was separated and the organic layer was washed with saturated aqueous sodium bicarbonate and brine, dried and concentrated in vacuo to give additional amount of the product (0.26 g, total 88%) as a colorless powder: ¹H NMR (400 MHz, CDCl₃) δ 1.40-1.54 (2H, m), 1.54-1.67 (2H, m), 1.98-2.14 (4H, m), 2.24-2.38 (2H, m), 3.68 (3H, s), 7.45 (2H, t, J=7.6 Hz), 7.55 (1H, t, J=7.6 Hz), 7.81 (2H, d, J=7.6 Hz), 8.78 (1H, d, J=6.4 Hz), 9.05 (1H, d, J=6.4 Hz).

The compound of the following structure was prepared from trans-4-(methoxycarbonyl)cyclohexanecarboxylic acid and nicotinohydrazide, using a similar method to that described above.

| Structure | Name | Physical Data |
|---|---|---|
| 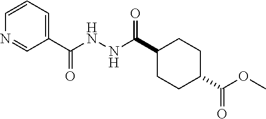 | trans-methyl 4- (N′-(pyridine- 3-carbonyl)- hydrazino- carbonyl) cyclohexane- carboxylate | ¹H NMR (400 MHz, CDCl₃) δ 1.34-1.70 (4H, m), 1.96-2.18 (4H, m), 2.22-2.40 (2H, m), 3.68 (3H, s), 7.40 (1H, dd, J = 4.8, 7.6 Hz), 8.11 (1H, d, J = 7.6 Hz), 8.63 (1H, s), 8.77 (1H, d, J = 4.8 Hz), 9.04 (1H, d, J = 1.6Hz); m/z (APCI pos) 306.1 (100%) [M + H]. |

### Step 2

A mixture of trans-methyl 4-[(2-benzoylhydrazino)carbonyl]cyclohexanecarboxylate (0.71 g, 2.35 mmol), phosphorus oxychloride (1.65 mL, 2.35 mmol) and CH₃CN (20 mL) was stirred at 80° C. for 4 h. After cooling to room temperature, the mixture was concentrated in vacuo. The residue was diluted with AcOEt, and the mixture was washed with saturated aqueous sodium bicarbonate and brine, dried, and concentrated in vacuo. The residue was purified by chromatography on silica (hexanes/AcOEt 2/1) to give trans-methyl 4-(5-phenyl-1,3,4-oxadiazol-2-yl)cyclohexanecarboxylate (0.61 g, 91%) as a pale yellow solid: ¹H NMR (400 MHz, CDCl₃) δ 1.55-1.79 (4H, m), 2.18 (2H, dd, J=3.2, 13.6 Hz), 2.30 (2H, dd, J=3.2, 13.6 Hz), 2.41 (1H, m), 2.97 (1H, m), 3.71 (3H, s), 7.46-7.56 (3H, m), 8.00-8.08 (2H, m); m/z (APCI pos) 287.1 (100%) [M+H].

The compound of the following structure was prepared from the corresponding diacylhydrazine, using a similar method to that described above.

| Structure | Name | Physical Data |
|---|---|---|
| 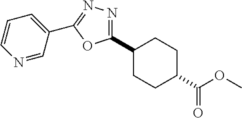 | trans-methyl 4- (5-(pyridin-3- yl)-1,3,4- oxadiazol-2-yl) cyclohexanecarboxylate | ¹H NMR (400 MHz, CDCl₃) δ 1.54-1.80 (4H, m), 2.14-2.24 (2H, m), 2.28-2.36 (2H, m), 2.42 (1H, m), 3.00 (1H, m), 3.71 (3H, s), 7.46 (1H, dd, J = 0.8, 8.0 Hz), 8.34 (1H, dt, J = 2.0, 8.0 Hz), 8.77 (1H, dd, J = 2.0, 4.8 Hz), 9.25 (1H, d, J = 2.0 Hz); m/z (APCI pos) 288.2 (100%) [M + H]. |

### trans-Methyl 4-(5-isopropyl-1,3,4-oxadiazol-2-yl)cyclohexanecarboxylate

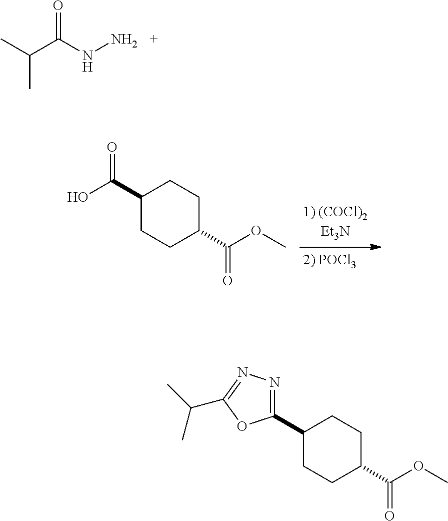

A solution of oxalyl chloride (0.26 mL, 2.95 mmol) in DCM (3 mL) was added dropwise over 20 min to a mixture of trans-4-(methoxycarbonyl)cyclohexanecarboxylic acid (0.50 g, 2.69 mmol), 2 drops of DMF and DCM (10 mL) at room temperature. After stirring for 30 min at room temperature, the mixture was concentrated in vacuo to dryness to give trans-methyl 4-(chlorocarbonyl)cyclohexanecarboxylate as a colorless oil. Then, isobutyrohydrazide (0.30 g, 2.95 mmol) was added to a mixture of this oil, triethylamine (0.75 mL, 5.37 mmol) and DCM (10 mL), and the mixture was stirred for 18 h at room temperature. The mixture was washed with saturated aqueous sodium bicarbonate and brine, dried and concentrated in vacuo. A mixture of the residue, phosphorus oxychloride (0.28 mL, 2.95 mmol) and CH₃CN (10 mL) was stirred at 80° C. for 16 h. After cooling, the mixture was diluted with AcOEt, washed with saturated aqueous sodium bicarbonate and brine, dried and concentrated in vacuo. The residue was purified by chromatography on silica (hexanes/AcOEt 2/1) to give trans-methyl 4-(5-isopropyl-1,3,4-oxadiazol-2-yl)cyclohexanecarboxylate (0.40 g, 60%) as colorless crystals: ¹H NMR (400 MHz, CDCl₃) δ 1.37 (6H, d, J=7.2 Hz), 1.50-1.70 (4H, m), 2.08-2.18 (2H, m), 2.18-2.28 (2H, m), 2.37 (1H, m), 2.85 (1H, m), 3.15 (1H, m), 3.69 (3H, s); m/z (APCI pos) 253.1 (100%) [M+H].

The compound of the following structure was prepared from trans-4-(methoxycarbonyl)cyclohexanecarboxylic acid and picolinohydrazide, using a similar method to that described above.

| Structure | Name | Physical Data |
|---|---|---|
| 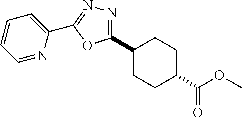 | trans-methyl 4- (5-(pyridin-2- yl)-1,3,4- oxadiazol-2-yl) cyclohexanecarboxylate | ¹H NMR (400 MHz, CDCl₃) δ 1.54-1.68 (2H, m), 1.70-1.84 (2H, m), 2.12-2.22 (2H, m), 2.28-2.38 (2H, m), 2.41 (1H, m), 3.02 (1H, m), 3.70 (3H, s), 7.45 (1H, dd, J = 5.2, 6.8 Hz), 7.88 (1H, t, J = 8.0 Hz), 8.25 (1H, d, J = 8.0 Hz), 8.78 (1H, d, J = 5.2 Hz); m/z (APCI pos) 288.1 (100%) [M + H]. |

### trans-Methyl 4-(5-isopropyl-1,3,4-thiadiazol-2-yl)cyclohexanecarboxylate

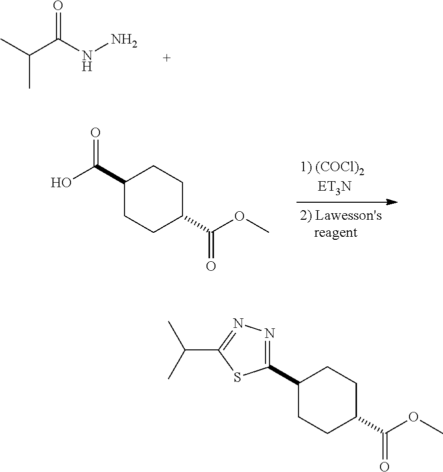

A solution of oxalyl chloride (0.26 mL, 2.95 mmol) in DCM (3 mL) was added dropwise over 20 min to a mixture of trans-4-(methoxycarbonyl)cyclohexanecarboxylic acid (0.50 g, 2.69 mmol), 2 drops of DMF and DCM (10 mL) at room temperature. After stirring for 30 min at room temperature, the mixture was concentrated in vacuo to dryness to give trans-methyl 4-(chlorocarbonyl)cyclohexanecarboxylate as a colorless oil. Isobutyrohydrazide (0.30 g, 2.95 mmol) was added to a mixture of the oil, triethylamine (0.75 mL, 5.37 mmol) and DCM (10 mL), and the mixture was stirred for 18 h at room temperature. The mixture was washed with water and brine, dried and concentrated in vacuo. To the residue, in toluene (10 mL), was added Lawesson's reagent (0.65 g, 1.61 mmol) and the mixture was stirred at 100° C. for 16 h. After cooling, the mixture was diluted with AcOEt, washed with saturated aqueous NaHCO₃ and brine, dried and concentrated in vacuo. The residue was purified by chromatography on silica (hexanes/AcOEt 2/1) to give trans-methyl 4-(5-isopropyl-1,3,4-thiadiazol-2-yl)cyclohexanecarboxylate (0.23 g, 32%) as colorless crystals: ¹H NMR (400 MHz, CDCl₃) δ 1.42 (6H, d, J=6.8 Hz), 1.52-1.68 (4H, m), 2.06-2.20 (2H, m), 2.20-2.32 (2H, m), 2.38 (1H, m), 3.11 (1H, m), 3.43 (1H, m), 3.69 (3H, s).

### trans-Methyl 4-(hydrazinocarbonyl)cyclohexanecarboxylate hydrochloride

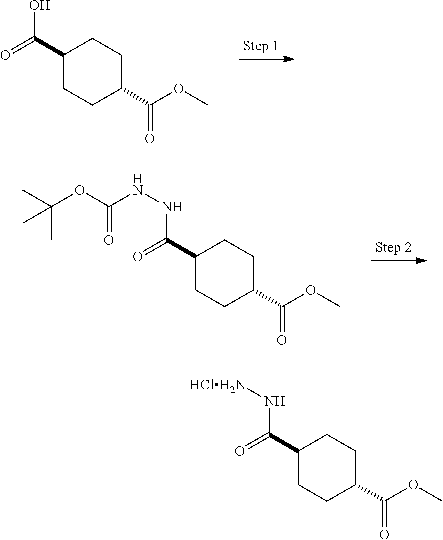

### Step 1

A solution of oxalyl chloride (2.603 mL, 29.54 mmol) in DCM (30 mL) was added dropwise over 20 min to a mixture of trans-4-(methoxycarbonyl)cyclohexanecarboxylic acid (5.00 g, 26.85 mmol), 2 drops of DMF and DCM (100 mL) at room temperature. After stirring for 30 minutes at room temperature, the mixture was concentrated in vacuo to dryness to give trans-methyl 4-(chlorocarbonyl)cyclohexanecarboxylate as a colorless oil. Boc-hydrazine (3.904 g, 29.54 mmol) was added to a mixture of this oil, triethylamine (7.485 mL, 53.70 mmol) and DCM (10 mL), and the mixture was stirred for 18 h at room temperature. The mixture was washed with water and brine, dried and concentrated in vacuo to give trans-tert-butyl 2-((4-(methoxycarbonyl)cyclohexyl)carbonyl)hydrazinecarboxylate as a tan solid.

### Step 2

trans-tert-Butyl 2-((4-(methoxycarbonyl)cyclohexyl)carbonyl)hydrazinecarboxylate from the previous step (8.065 g, 26.85 mmol) was dissolved in DCM (100 mL), and TFA (20 mL) was added. The mixture was stirred at room temperature for 18 hours then it was concentrated to dryness to give a residue which was dissolved in DCM (10 mL). 1N HCl in Et₂O (200 mL) was added and the resulting precipitate was filtered and dried under vacuum to yield trans-methyl 4-(hydrazinocarbonyl)cyclohexanecarboxylate hydrochloride (4.95 g, 78% over 2 steps) as a tan solid.

### trans-Methyl 4-(5-oxo-4,5-dihydro-1,3,4-oxadiazol-2-yl)cyclohexanecarboxylate

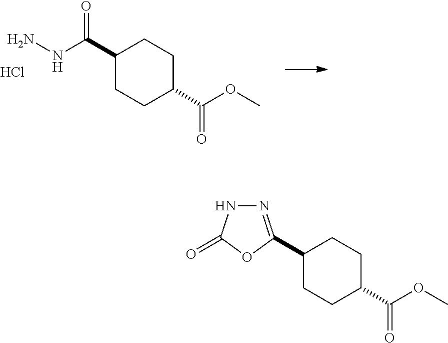

To a mixture of trans-methyl 4-(hydrazinocarbonyl)cyclohexanecarboxylate hydrochloride (0.30 g, 1.27 mmol), triethylamine (0.37 mL, 2.66 mmol) and THF (6 mL) was added 1,1′-carbonyldiimidazole (0.26 g, 1.58 mmol) at room temperature. After being stirred for 18 hours, the mixture was diluted with AcOEt, washed with 1N HCl and brine, dried and concentrated in vacuo. The residue was purified by chromatography on silica (hexanes/AcOEt 4/3) to give trans-methyl 4-(5-oxo-4,5-dihydro-1,3,4-oxadiazol-2-yl)cyclohexanecarboxylate (0.22 g, 76%) as a colorless solid: ¹H NMR (400 MHz, CDCl₃) δ 1.44-1.62 (4H, m), 2.04-2.22 (4H, m), 2.33 (1H, m), 2.57 (1H, m), 3.69 (3H, s), 8.58 (1H, s); m/z (APCI neg) 225.1 (100%) [M−H].

The compound of the following structure was prepared from the corresponding hydrazide, using a similar method to that described above.

| Structure | Name | Physical Data |
|---|---|---|
| 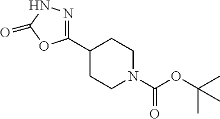 | tert-butyl 4-(5-oxo-4,5- dihydro-1,3,4-oxadiazol-2- yl)piperidine-1-carboxylate | White solid (87%); ¹H NMR (400 MHz, DMSO-d₆) δ 1.39 (s, 9H), 1.38-1.50 (m, 2H), 1.83-1.91 (m, 2H), 2.80-2.95 (m, 3H), 3.84-3.92 (m, 2H), 12.10 (br, 1H); m/z (APCI pos) 170.2 (100%) [M + H]. |

### trans-Methyl 4-(5-phenyloxazol-2-yl)cyclohexanecarboxylate

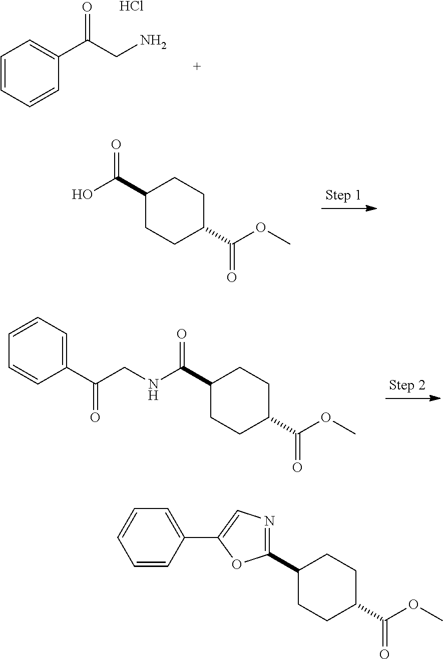

### Step 1

A solution of oxalyl chloride (0.26 mL, 2.95 mmol) in DCM (3 mL) was added dropwise over 20 min to a mixture of trans-4-(methoxycarbonyl)cyclohexanecarboxylic acid (0.50 g, 2.69 mmol), 2 drops of DMF and DCM (10 mL) at room temperature. After stirring for 30 minutes at room temperature, the mixture was concentrated in vacuo to dryness to give trans-methyl 4-(chlorocarbonyl)cyclohexanecarboxylate as a colorless oil. Then, a mixture of this oil, 2-amino-1-phenylethanone hydrochloride (0.51 g, 2.95 mmol) and pyridine (10 mL) was heated to reflux for 30 minutes. After cooling to room temperature, the mixture was concentrated in vacuo and the residue was partitioned between AcOEt and water. The organic layer was washed with brine, dried and concentrated in vacuo. The residue was purified by chromatography on silica (hexanes/AcOEt 2/1->1/2) to give trans-methyl 4-(2-oxo-2-phenylethylcarbamoyl)cyclohexanecarboxylate (0.62 g, 76%) as a yellow solid: ¹H NMR (400 MHz, CDCl₃) δ 1.40-1.66 (4H, m), 2.00-2.14 (4H, m), 2.24 (1H, m), 2.33 (1H, m), 3.68 (3H, s), 4.76 (2H, d, J=4.4 Hz), 6.58 (1H, br), 7.51 (2H, t, J=7.6 Hz), 7.63 (1H, t, J=7.6 Hz), 7.99 (2H, d, J=7.6 Hz); m/z (APCI pos) 304.0 (100%) [M+H].

### Step 2

A mixture of trans-methyl 4-((2-oxo-2-phenylethyl)carbamoyl)cyclohexanecarboxylate (0.62 g, 2.03 mmol), phosphorus oxychloride (0.21 mL, 2.24 mmol) and CH₃CN (10 mL) was heated to 80° C. for 2.5 hours. After cooling to room temperature, the mixture was diluted with AcOEt, washed with saturated aqueous NaHCO₃ and brine, dried and concentrated in vacuo. The residue was purified by chromatography on silica (hexanes/AcOEt 2/1) to give trans-methyl 4-(5-phenyloxazol-2-yl)cyclohexanecarboxylate (0.46 g, 79%) as a pale yellow solid: ¹H NMR (400 MHz, CDCl₃) δ 1.52-1.74 (4H, m), 2.10-2.20 (2H, m), 2.20-2.33 (2H, m), 2.39 (1H, m), 2.85 (1H, m), 3.70 (3H, s), 7.22 (1H, s), 7.31 (1H, t, J=7.6 Hz), 7.41 (2H, t, J=7.6 Hz), 7.61 (2H, d, J=7.6 Hz); m/z (APCI pos) 286.2 (100%) [M+H].

### trans-Methyl 4-(5-(2,2-difluorocyclopropyl)-1,3,4-oxadiazol-2-yl)cyclohexanecarboxylate

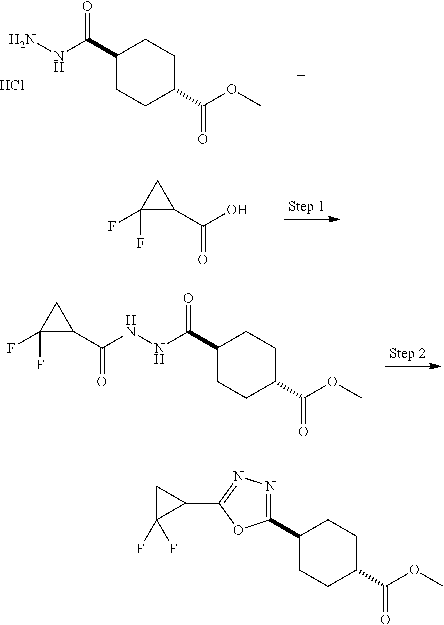

### Step 1

A mixture of trans-methyl 4-(hydrazinocarbonyl)cyclohexanecarboxylate hydrochloride (0.30 g, 1.27 mmol), 2,2-difluorocyclopropanecarboxylic acid (0.18 g, 1.39 mmol), EDAC.HCl (0.36 g, 1.90 mmol), HOBt.H₂O (0.29 g, 1.90 mmol), triethylamine (0.53 mL, 3.8 mmol) and CH₃CN (10 mL) was stirred for 16 hours at room temperature. The mixture was diluted with AcOEt, washed successively with 1N HCl, saturated aqueous NaHCO₃ and brine, dried and concentrated in vacuo to give trans-methyl 4-(N′-(2,2-difluorocyclopropanoyl)hydrazinocarbonyl)cyclohexanecarboxylate (0.36 g, 93%) as a beige solid: ¹H NMR (400 MHz, CDCl₃) δ 1.36-1.70 (4H, m), 1.70-1.88 (2H, m), 1.92-2.50 (7H, m), 3.67 (3H, s), 8.25 (1H, s), 8.71 (1H, s); m/z (APCI neg) 303.2 (100%) [M−H].

Compound of the following structure was prepared from 4,4,4-trifluorobutanoic acid and trans-methyl 4-(hydrazinocarbonyl)cyclohexanecarboxylate hydrochloride, using a similar method to that described above.

| Structure | Name | Physical Data |
|---|---|---|
| 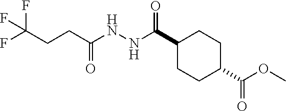 | trans-methyl 4- (N′-(4,4,4- trifluorobutano yl)hydrazino- carbonyl) cyclohexane- carboxylate | ¹H NMR (400 MHz, DMSO- d₆) δ 1.20-1.48 (4H, m), 1.70-1.82 (2H, m), 1.88-1.98 (2H, m), 2.17 (1H, m), 2.29 (1H, m), 2.35-2.58 (4H, m), 3.59 (3H, s), 9.75 (1H, d, J = 2.0 Hz), 9.89 (1H, d, J = 2.0 Hz). |

### Step 2

A mixture of trans-methyl 4-(N′-(2,2-difluorocyclopropanoyl)hydrazinocarbonyl)cyclohexanecarboxylate (0.36 g, 1.18 mmol), phosphorus oxychloride (0.12 mL, 1.30 mmol) and CH₃CN (10 mL) was heated at 80° C. for 16 hours. After cooling to room temperature, the mixture was diluted with AcOEt, washed with saturated aqueous sodium bicarbonate and brine, dried and concentrated in vacuo. The residue was purified by chromatography on silica (hexanes/AcOEt 2/1->1/2) to give trans-methyl 4-(5-(2,2-difluorocyclopropyl)-1,3,4-oxadiazol-2-yl)cyclohexanecarboxylate (0.13 g, 39%) as a pale yellow solid: ¹H NMR (400 MHz, CDCl₃) δ 1.50-1.72 (4H, m), 2.05 (1H, m), 2.10-2.30 (4H, m), 2.38 (1H, m), 2.90 (1H, m), 3.64 (1H, t, J=10.0 Hz), 3.70 (3H, s), 3.87 (1H, t, J=12.0 Hz).

Compound of the following structure was prepared from trans-methyl 4-(N′-(4,4,4-trifluorobutanoyl)hydrazinocarbonyl)cyclohexanecarboxylate, using a similar method to that described above.

| Structure | Name | Physical Data |
|---|---|---|
| 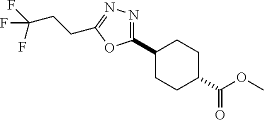 | trans-methyl 4- (5-(3,3,3- trifluoropropyl)- 1,3,4- oxadiazol-2- yl)cyclohexane- carboxylate | ¹H NMR (400 MHz, CDCl₃) δ 1.50-1.70 (4H, m), 2.08-2.18 (2H, m), 2.18-2.28 (2H, m), 2.38 (1H, m), 2.54-2.74 (2H, m), 2.87 (1H, m), 3.04- 3.16 (2H, m), 3.70 (3H, s); m/z (APCI pos) 307.1 (100%) [M + H]. |

### trans-Methyl 4-(5-(tetrahydro-2H-pyran-4-yl)-1,3,4-oxadiazol-2-yl)cyclohexanecarboxylate

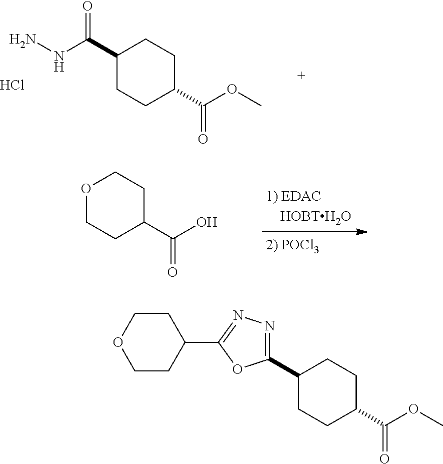

A mixture of trans-methyl 4-(hydrazinocarbonyl)cyclohexanecarboxylate hydrochloride (0.30 g, 1.27 mmol), tetrahydro-2H-pyran-4-carboxylic acid (0.18 g, 1.39 mmol), EDAC.HCl (0.36 g, 1.90 mmol), HOBt.H₂O (0.29 g, 1.90 mmol), triethylamine (0.53 mL, 3.8 mmol) and CH₃CN (10 mL) was stirred for 16 hours at room temperature. The mixture was diluted with CHCl₃/iPrOH (3/1), washed successively with 1N HCl, saturated aqueous sodium bicarbonate and brine, dried and concentrated in vacuo to give a beige solid (0.17 g). A mixture of this solid, phosphorus oxychloride (0.12 mL, 1.27 mmol) and CH₃CN (10 mL) was heated at 80° C. for 16 hours. The mixture was diluted with AcOEt, washed with saturated aqueous sodium bicarbonate and brine, dried and concentrated in vacuo. The residue was purified by chromatography on silica (hexanes/AcOEt 1/2->1/4) to give trans-methyl 4-(5-(tetrahydro-2H-pyran-4-yl)-1,3,4-oxadiazol-2-yl)cyclohexane-carboxylate (0.12 g, 31%) as a colorless solid: ¹H NMR (400 MHz, CDCl₃) δ 1.50-1.70 (4H, m), 1.88-2.04 (4H, m), 2.10-2.18 (2H, m), 2.18-2.27 (2H, m), 2.37 (1H, m), 2.86 (1H, m), 3.13 (1H, m), 3.48-3.60 (2H, m), 3.69 (3H, s), 3.98-4.08 (2H, m); m/z (APCI pos) 295.2 (100%) [M+H].

Compounds of the following structures were prepared from the corresponding acids and trans-methyl 4-(hydrazinocarbonyl)cyclohexanecarboxylate hydrochloride, using a similar method to that described above.

| Structure | Name | Physical Data |
|---|---|---|
| 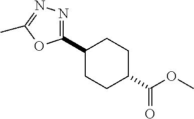 | trans-methyl 4- (5-methyl- 1,3,4- oxadiazol-2-yl) cyclohexanecarboxylate | m/z (APCI pos) 225 [M + H]. |
| 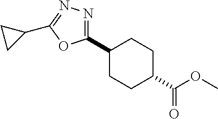 | trans-methyl 4- (5-cyclopropyl- 1,3,4- oxadiazol-2-yl) cyclohexanecarboxylate | ¹H NMR (400 MHz, CDCl₃) δ 1.06-1.14 (m, 4H), 2.05-2.15 (m, 3H), 2.17-2.21 (m, 2H), 2.32-2.40 (m, 1H), 2.77-2.85 (m, 1H), 3.69 (s, 3H); m/z (APCI pos) 267 [M + H]. |
| 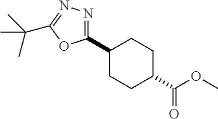 | trans-methyl 4- (5-tert-butyl- 1,3,4- oxadiazol-2-yl) cyclohexanecarboxylate | ¹H NMR (400 MHz, CDCl₃) δ 1.40 (s, 9H), 1.52- 1.68 (m, 4H), 2.12 (br, 1H), 2.15 (br, 1H), 2.21-2.24 (m, 2H), 2.35-2.41 (m, 1H), 2.82-2.88 (m, 1H), 3.69 (s, 3H); m/z (APCI pos) 251 [M + H]. |

### Methyl 1-(5-phenyl-1,3,4-oxadiazol-2-yl)piperidine-4-carboxylate

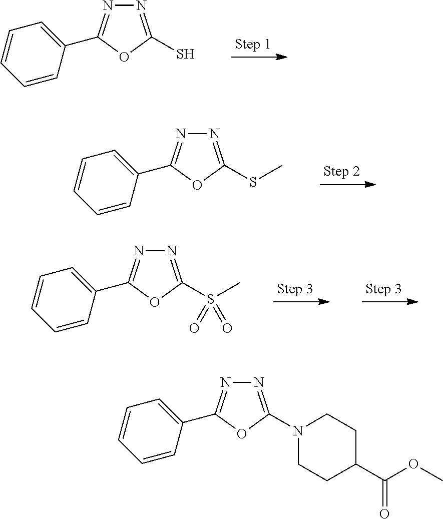

### Step 1

A mixture of 5-phenyl-1,3,4-oxadiazole-2-thiol (3.0 g, 16.8 mmol), potassium carbonate (3.49 g, 25.3 mmol), iodomethane (1.16 mL, 18.52 mmol) and DMF (30 mL) was stirred for 16 hours at room temperature. The mixture was diluted with AcOEt, washed successively with water, 1N HCl and brine, dried and concentrated in vacuo. The residue was purified by chromatography on silica (hexanes/AcOEt 3/1) to give 2-(methylthio)-5-phenyl-1,3,4-oxadiazole (3.06 g, 95%) as colorless crystals: ¹H NMR (400 MHz, CDCl₃) δ 2.79 (3H, s), 7.46-7.56 (3H, m), 7.98-8.04 (2H, m); m/z (APCI pos) 193.0 (100%) [M+H].

### Step 2

To a mixture of 2-(methylthio)-5-phenyl-1,3,4-oxadiazole (3.06 g, 15.9 mmol), formic acid (6.01 mL, 159 mmol) and acetone (100 mL) was added potassium permanganate (6.29 g, 39.8 mmol) in portions at 0° C. After being stirred for 16 hours at room temperature, insoluble materials were removed by filtration. The filtrate was concentrated in vacuo and the residue was purified by chromatography on silica (hexanes/AcOEt 3/1->2/1) to give 2-(methylsulfonyl)-5-phenyl-1,3,4-oxadiazole (0.21 g, 6%) as a colorless solid: ¹H NMR (400 MHz, CDCl₃) δ 3.53 (3H, s), 7.57 (2H, t, J=7.2 Hz), 7.65 (1H, t, J=7.2 Hz), 8.14 (2H, d, J=7.2 Hz).

### Step 3

A mixture of 2-(methylsulfonyl)-5-phenyl-1,3,4-oxadiazole (0.21 g, 0.94 mmol), methyl piperidine-4-carboxylate (0.25 mL, 1.88 mmol) and EtOH (10 mL) was heated to reflux for 4 hours. After cooling to room temperature, the mixture was concentrated in vacuo. The residue was purified by chromatography on silica (hexanes/AcOEt 3/1->1/2) to give methyl 1-(5-phenyl-1,3,4-oxadiazol-2-yl)piperidine-4-carboxylate (0.055 g, 20%) as a colorless oil: ¹H NMR (400 MHz, CDCl₃) δ 1.80-1.90 (2H, m), 2.00-2.10 (2H, m), 2.57 (1H, m), 3.16-3.26 (2H, m), 3.72 (3H, s), 4.05 (2H, dt, J=4.0, 13.2 Hz), 7.38-7.50 (3H, m), 7.88-7.96 (2H, m); m/z (APCI pos) 288.2 (100%) [M+H].

### trans-Methyl 4-(5-phenyl-1,2,4-oxadiazol-3-yl)cyclohexanecarboxylate

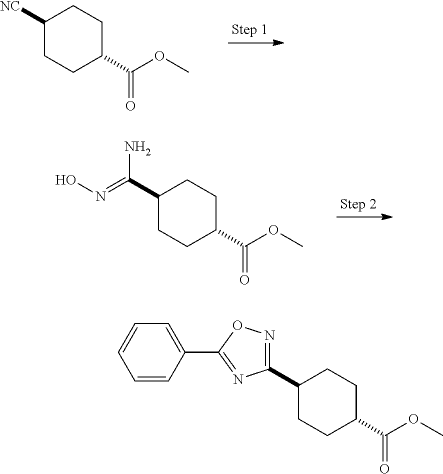

### Step 1

To a solution of sodium bicarbonate (2.0 g, 24 mmol) in water (30 mL) was added dropwise a solution of hydroxylamine hydrochloride (1.7 g, 24 mmol) in water (5 mL). The mixture was added to a solution of trans-methyl 4-cyanocyclohexanecarboxylate (4.0 g, 24 mmol) in 95% EtOH (200 mL) and the reaction mixture was heated to reflux for 16 hours. After cooling to room temperature, the mixture was concentrated in vacuo. The residue was diluted with CHCl₃/iPrOH (3/1), and the mixture was washed with water and brine, dried and concentrated in vacuo to give trans-methyl 4-(N′-hydroxycarbamimidoyl)cyclohexanecarboxylate (4.1 g, 86%) as a colorless solid: ¹H NMR (400 MHz, CDCl₃) δ 1.34-1.70 (4H, m), 1.94-2.54 (6H, m), 3.67 and 3.68 (total 3H, s for each), 4.49 (1H, br); m/z (APCI pos) 201.2 (100%) [M+H].

### Step 2

A mixture of trans-methyl 4-(N′-hydroxycarbamimidoyl)cyclohexanecarboxylate (0.53 g, 2.65 mmol), benzoyl chloride (0.37 mL, 3.18 mmol), pyridine (0.28 mL, 3.44 mmol) and xylene (10 mL) was heated at 140° C. for 1 hour. After cooling to room temperature, the mixture was diluted with AcOEt, washed with saturated aqueous sodium bicarbonate and brine, dried and concentrated in vacuo. The residue was purified by chromatography on silica (hexanes/AcOEt 6/1) to give trans-methyl 4-(5-phenyl-1,2,4-oxadiazol-3-yl)cyclohexanecarboxylate (0.38 g, 50%) as colorless crystals: ¹H NMR (400 MHz, CDCl₃) δ 1.54-1.76 (4H, m), 2.10-2.19 (2H, m), 2.19-2.28 (2H, m), 2.41 (1H, m), 2.86 (1H, m), 3.70 (3H, s), 7.49-7.56 (2H, m), 7.59 (1H, m), 8.09-8.16 (2H, m); m/z (APCI pos) 287.3 (5%) [M+H].

### trans-Methyl 4-(5-oxo-4,5-dihydro-1,2,4-oxadiazol-3-yl)cyclohexanecarboxylate

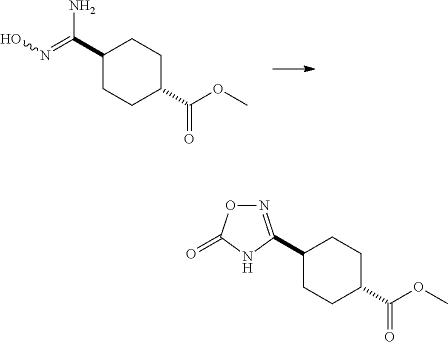

A mixture of trans-methyl 4-(N′-hydroxycarbamimidoyl)cyclohexanecarboxylate (0.50 g, 2.50 mmol), 1,1′-carbonyldiimidazole (0.49 g, 3.00 mmol) and 1,4-dioxane (10 mL) was heated to reflux for 30 minutes. After cooling to room temperature, the mixture was concentrated in vacuo. The residue was diluted with AcOEt, and the mixture was washed with 1N HCl and brine, dried and concentrated in vacuo. The residue was purified by chromatography on silica (hexanes/AcOEt 1/1->1/3) to give trans-methyl 4-(5-oxo-4,5-dihydro-1,2,4-oxadiazol-3-yl)cyclohexanecarboxylate (0.31 g, 55%) as a colorless solid: ¹H NMR (400 MHz, CDCl₃) δ 1.44-1.64 (4H, m), 2.06-2.22 (4H, m), 2.37 (1H, m), 2.63 (1H, m), 3.69 (3H, s), 10.37 (1H, s); m/z (APCI neg) 225.2 (80%) [M−H].

### trans-4-(5-Phenyl-1,3,4-oxadiazol-2-yl)cyclohexanecarboxylic acid

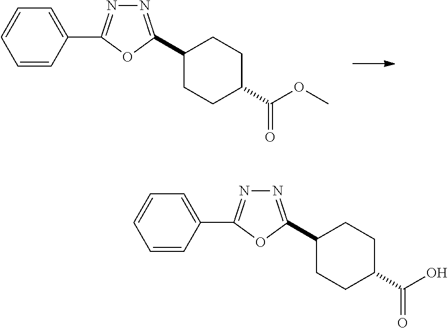

A mixture of trans-methyl 4-(5-phenyl-1,3,4-oxadiazol-2-yl)cyclohexanecarboxylate (0.61 g, 2.1 mmol), 1N NaOH (4.3 mL, 4.3 mmoL) and MeOH (12 mL) was stirred at 50° C. for 1.5 hours. After cooling, the mixture was concentrated in vacuo. The residue was partitioned between AcOEt and 1N HCl. The organic layer was washed with water and brine, dried and concentrated in vacuo. The resulting solid was collected, washed with hexanes/ether (1/1) and dried to give trans-4-(5-phenyl-1,3,4-oxadiazol-2-yl)cyclohexanecarboxylic acid (0.52 g, 89%) as a color less solid: ¹H NMR (400 MHz, CDCl₃) δ 1.58-1.82 (4H, m), 2.18-2.28 (2H, m), 2.29-2.38 (2H, m), 2.47 (1H, m), 2.99 (1H, m), 7.46-7.58 (3H, m), 8.00-8.08 (2H, m); m/z (APCI pos) 273.2 (100%) [M+H].

Compounds of the following structures were prepared from the corresponding esters, using a similar method to that described above.

| Structure | Name | Physical Data |
|---|---|---|
| 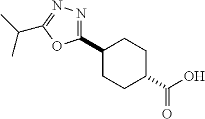 | trans-4-(5- isopropyl- 1,3,4- oxadiazol-2- yl)cyclohexane- carboxylic acid | ¹H NMR (400 MHz, CDCl₃) δ 1.37 (6H, d, J = 6.8 Hz), 1.52-1.72 (4H, m), 2.12-2.30 (4H, m), 2.42 (1H, m), 2.87 (1H, m), 3.16 (1H, m); m/z (APCI pos) 239.2 (100%) [M + H]. |
| 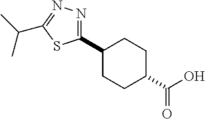 | trans-4-(5- isopropyl- 1,3,4- thiadiazol-2- yl)cyclohexane- carboxylic acid | ¹H NMR (400 MHz, CDCl₃) δ 1.42 (6H, d, J = 6.8 Hz), 1.54-1.72 (4H, m), 2.12-2.24 (2H, m), 2.24-2.34 (2H, m), 2.42 (1H, m), 3.13 (1H, m), 3.44 (1H, m). |
| 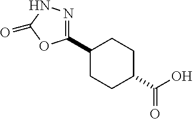 | trans-4-(5-oxo- 4,5-dihydro- 1,3,4- oxadiazol-2- yl)cyclohexane- carboxylic acid | ¹H NMR (400 MHz, DMSO- d₆) δ 1.32-1.50 (4H, m), 1.88-2.04 (4H, m), 2.22 (1H, m), 2.58 (1H, m), 12.05 (1H, s), 12.10 (1H, s); m/z (APCI neg) 211.1 (100%) [M − H]. |
| 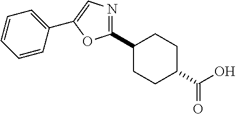 | trans-4-(5- phenyloxazol-2- yl)cyclohexane- carboxylic acid | ¹H NMR (400 MHz, CDCl₃) δ 1.56-1.76 (4H, m), 2.16-2.26 (2H, m), 2.26-2.36 (2H, m), 2.44 (1H, m), 2.87 (1H, m), 7.24 (1H, s), 7.31 (1H, m), 7.38-7.44 (2H, m), 7.59-7.64 (2H, m); m/z (APCI pos) 272.2 (100%) [M + H]. |
| 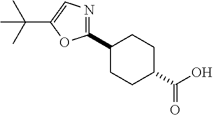 | trans-4-(5- tert- butyloxazol-2- yl)cyclohexane- carboxylic acid | ¹H NMR (400 MHz, CDCl3) δ 1.27 (9H, s), 1.48- 1.70 (4H, m), 2.08-2.28 (4H, m), 2.40 (1H, m), 2.75 (1H, m), 6.59 (1H, s); m/z (APCI pos) 252.2 (100%) [M + H]. |
| 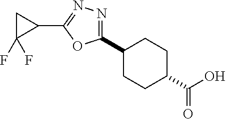 | trans-4-(5- (2,2- difluorocyclo- propyl)-1,3,4- oxadiazol-2- yl)cyclohexane- carboxylic acid | m/z (APCI neg) 271.1 (100%) [M − H]. |
| 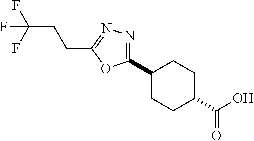 | trans-4-(5- (3,3,3- trifluoropropyl)- 1,3,4- oxadiazol-2- yl)cyclohexane- carboxylic acid | ¹H NMR (400 MHz, CDCl3) δ 1.52-1.72 (4H, m), 2.14-2.30 (4H, m), 2.42 (1H, m), 2.58-2.74 (2H, m), 2.88 (1H, m), 3.10 (2H, t, J = 8.0 Hz), 10.74 (1H, br); m/z (APCI pos) 293.2 (100%) [M + H]. |
| 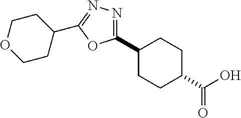 | trans-4-(5- (tetrahydro-2H- pyran-4-yl)- 1,3,4- oxadiazol-2- yl)cyclohexane- carboxylic acid | ¹H NMR (400 MHz, CDCl₃) δ 1.52-1.72 (4H, m), 1.88-2.06 (4H, m), 2.14-2.30 (4H, m), 2.42 (1H, m), 2.88 (1H, m), 3.14 (1H, m), 3.48-3.60 (2H, m), 4.04 (2H, d, J = 11.6 Hz), 10.21 (1H, br); m/z (APCI pos) 281.2 (100%) [M + H]. |
| 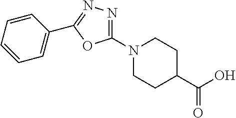 | 1-(5-phenyl- 1,3,4- oxadiazol-2- yl)piperidine- 4-carboxylic acid | ¹H NMR (400 MHz, CDCl₃) δ 1.82-1.96 (2H, m), 2.06-2.14 (2H, m), 2.63 (1H, m), 3.20-3.30 (2H, m), 4.02-4.12 (2H, m), 7.42-7.50 (3H, m), 7.88-7.96 (2H, m); m/z (APCI pos) 274.2 (100%) [M + H]. |
| 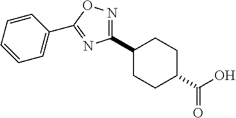 | trans-4-(5- phenyl-1,2,4- oxadiazol-3- yl)cyclohexane- carboxylic acid | ¹H NMR (400 MHz, CDCl₃) δ 1.56-1.80 (4H, m), 2.16-2.30 (4H, m), 2.46 (1H, m), 2.88 (1H, m), 7.48-7.56 (2H, m), 7.59 (1H, m), 8.10-8.16 (2H, m), 10.74 (1H, br); m/z (APCI neg) 271.1 (100%) [M − H]. |
| 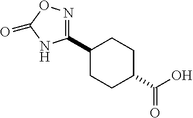 | trans-4-(5-oxo- 4,5-dihydro- 1,2,4- oxadiazol-3- yl)cyclohexane- carboxylic acid | ¹H NMR (400 MHz, DMSO- d₆) δ 1.32-1.50 (4H, m), 1.88-2.04 (4H, m), 2.19 (1H, m), 2.56 (1H, m), 12.13 (1H, s), 12.18 (1H, s); m/z (APCI neg) 211.2 [M − H]. |
| 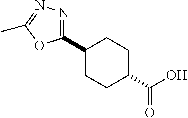 | trans-4-(5- methyl-1,3,4- oxadiazol-2- yl)cyclohexane- carboxylic acid | m/z (APCI pos) 211 [M + H]. |
| 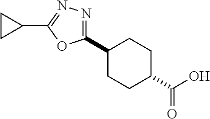 | trans-4-(5- cyclopropyl- 1,3,4- oxadiazol-2- yl)cyclohexane- carboxylic acid | m/z (APCI neg) 251 [M − H]. |
| 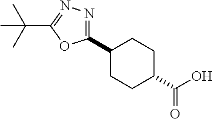 | trans-4-(5- tert-butyl- 1,3,4- oxadiazol-2- yl)cyclohexane- carboxylic acid | m/z (APCI pos) 237 [M + H]. |

### trans-Methyl 4-(5-((dimethylamino)methyl)-1,3,4-oxadiazol-2-yl)cyclohexanecarboxylate

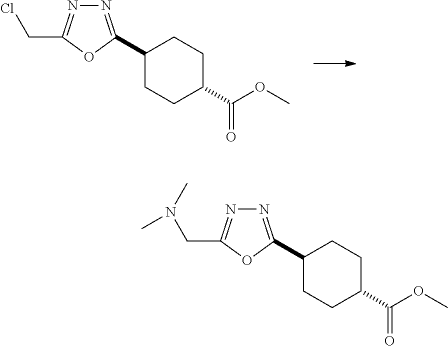

A mixture of trans-methyl 4-(5-(chloromethyl)-1,3,4-oxadiazol-2-yl)cyclohexanecarboxylate (0.27 g, 1.03 mmol), potassium carbonate (0.43 g, 3.08 mmol) and dimethylamine (THF solution, 2M, 3.08 mL, 6.17 mmol) in THF (10 ml) was stirred at 50° C. for 16 h. After cooling to room temperature, the mixture was concentrated in vacuo. The residue was diluted with CHCl₃/iPrOH (3/1), and the mixture was washed successively with saturated aqueous sodium bicarbonate and brine, dried and concentrated in vacuo. The residue was purified by chromatography on silica (AcOEt/MeOH 10/1) to give trans-methyl 4-(5-((dimethylamino)methyl)-1,3,4-oxadiazol-2-yl)cyclohexanecarboxylate (0.16 g, y. 58%) as pale yellow crystals. ¹H NMR (400 MHz, CDCl₃) δ 1.50-1.72 (4H, m), 2.08-2.20 (2H, m), 2.20-2.30 (2H, m), 2.34 (6H, s), 2.37 (1H, m), 2.89 (1H, m), 3.69 (3H, s), 3.72 (2H, s); m/e (APCI pos) 268.0 (100%) (M+H).

### trans-4-(5-((Dimethylamino)methyl)-1,3,4-oxadiazol-2-yl)cyclohexanecarboxylic acid

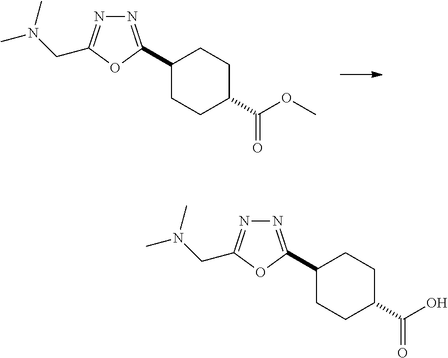

A mixture of trans-methyl 4-(5-((dimethylamino)methyl)-1,3,4-oxadiazol-2-yl)cyclohexanecarboxylate (0.16 g, 0.58 mmol), 1N NaOH (1.2 mL, 1.2 mmol) and MeOH (10 mL) was stirred at 50° C. for 3 h. After cooling to room temperature, the mixture was concentrated in vacuo. The residue was diluted with water, neutralized with 1N) HCl and extracted with CHCl₃/iPrOH (3/1). The Organic layer was washed with brine, dried and concentrated in vacuo to give trans-4-(5-((dimethylamino)methyl)-1,3,4-oxadiazol-2-yl)cyclohexanecarboxylic acid (0.13 g, 85%) as a beige solid. ¹H NMR (400 MHz, CDCl₃) δ 1.52-1.74 (4H, m), 2.14-2.30 (4H, m), 2.36 (6H, s), 2.39 (1H, m), 2.91 (1H, m), 3.76 (2H, s); m/e (APCI pos) 254.0 (100%) (M+H).

Compounds of the following structures were prepared from the corresponding esters, using a similar method to that described above.

| Structure | Name | Physical Data |
|---|---|---|
| 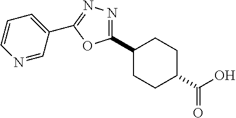 | trans-4-(5- (pyridin-3-yl)- 1,3,4- oxadiazol-2-yl) cyclohexanecarboxylic acid | ¹H NMR (400 MHz, CDCl₃) δ 1.58-1.84 (4H, m), 2.20-2.30 (2H, m), 2.30-2.40 (2H, m), 2.48 (1H, m), 3.02 (1H, m), 7.47 (1H, m), 8.36 (1H, dt, J = 2.0, 8.0 Hz), 8.78 (1H, dd, J = 1.6, 4.8 Hz), 9.25 (1H, dd, J = 0.8, 2.0 Hz); m/z (APCI pos) 274.2 (100%) [M + H]. |
| 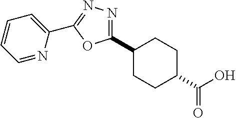 | trans-4-(5- (pyridin-2-yl)- 1,3,4- oxadiazol-2-yl) cyclohexanecarboxylic acid | ¹H NMR (400 MHz, CDCl₃) δ 1.56-1.72 (2H, m), 1.72-1.88 (2H, m), 2.18-2.28 (2H, m), 2.30-2.40 (2H, m), 2.47 (1H, m), 3.05 (1H, m), 7.47 (1H, m), 7.89 (1H, dt, J = 1.6, 8.0 Hz), 8.26 (1H, d, J = 8.0 Hz), 8.79 (1H, dd, J = 1.6, 8.8 Hz); m/z (APCI pos) 274.2 (100%) [M + H]. |

### Example E1

### N-(2-(4-Methoxycyclohexanecarboxamido)ethyl)-1-phenyl-3-(trifluoromethyl)-1H-pyrazole-4-carboxamide

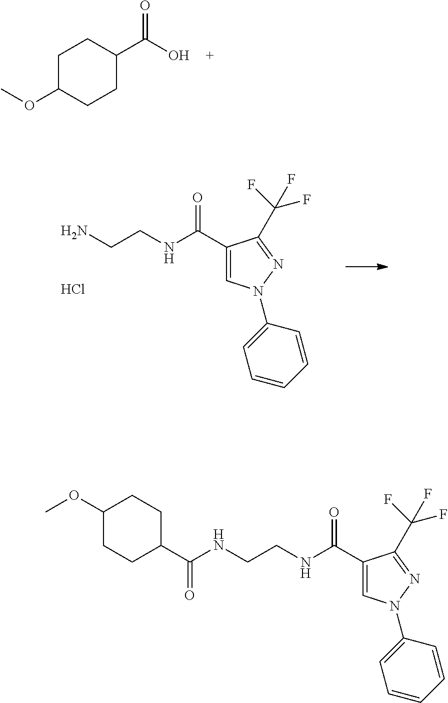

4-Methoxycyclohexanecarboxylic acid (0.095 g, 0.60 mmol), N-(2-aminoethyl)-1-phenyl-3-(trifluoromethyl)-1H-pyrazole-4-carboxamide hydrochloride (0.20 g, 0.60 mmol) and HATU (0.273 g, 0.72 mmol) were suspended in THF (10 mL). Diisopropylethylamine was then added (0.34 mL, 2.0 mmol). The mixture was stirred at room temperature for 5 hours. The solution was then poured over water and the resultant solid was filtered, dried, and recrystallized using EtOAc to afford 0.15 g (56% yield) of a 1:3 mixture (trans:cis) of N-(2-(4-methoxycyclohexanecarboxamido)ethyl)-1-phenyl-3-(trifluoromethyl)-1H-pyrazole-4-carboxamide.

¹H NMR (400 MHz, CDCl₃) δ 1.16-1.96 (m, 8H), 2.04-2.22 (m, 1H), 3.08-3.16 (m, 1H, trans-axial CH—OMe), 3.27 (s, 3H, cis-axial CH₃), 3.34 (s, 3H, trans-equatorial CH₃), 3.42 (t, J=1.6 Hz, 1H, cis-equitorial CH—OMe), 3.47-3.51 (m, 2H), 3.57-3.59 (m, 2H), 6.23 (br, 1H), 6.90 (br, 1H, trans NH) 7.01 (br, 1H, cis NH), 7.39-7.44 (m, 1H), 7.49-7.53 (m, 2H), 7.70-7.73 (m, 2H), 8.43 (s, 1H); m/z (APCI pos) 439.1, (100%) 439.1 [M+H].

### Example E2

### tert-Butyl 3-((2-(1-phenyl-3-(trifluoromethyl)-1H-pyrazole-4-carboxamido)ethyl)carbamoyl)piperidine-1-carboxylate

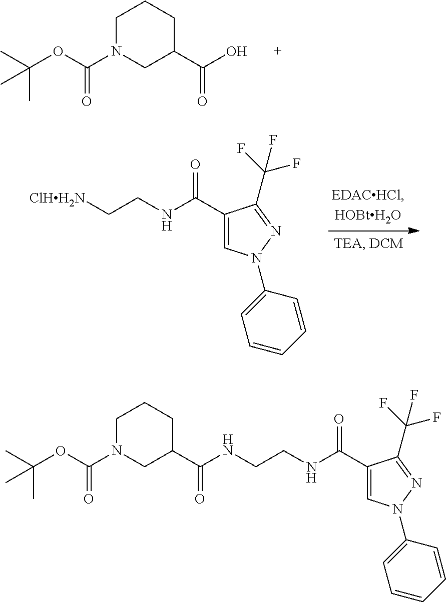

To N-(2-aminoethyl)-1-phenyl-3-(trifluoromethyl)-1H-pyrazole-4-carboxamide hydrochloride (1.17 g, 3.49 mmol) in DCM (200 mL) were added successively 1-(tert-butoxycarbonyl)piperidine-3-carboxylic acid (0.8 g, 3.49 mmol), EDAC.HCl (0.87 g, 4.5 mmol), HOBt.H₂O (0.66 g, 4.9 mmol) and triethylamine (1.06 g, 10.5 mmol). The mixture was stirred at room temperature for 12 hours then DCM (200 mL) was added. The organic layer was washed successively with 2N HCl (200 mL), 10% aqueous K₂CO₃ (200 mL) and brine (200 mL), and dried over MgSO₄. After concentration and purification by MPLC (DCM/MeOH 99/1), tert-butyl 3-((2-(1-phenyl-3-(trifluoromethyl)-1H-pyrazole-4-carboxamido)ethyl)carbamoyl)piperidine-1-carboxylate was isolated as a white solid (1.2 g, 67%): ¹H NMR (400 MHz, DMSO-d₆) δ 1.20-1.35 (m, 2H), 1.37 (s, 9H), 1.44-1.55 (m, 1H), 1.59-1.66 (m, 1H), 1.79-1.86 (m, 1H), 2.15-2.25 (m, 1H), 2.70 (br, 2H), 3.16-3.32 (m, 4H), 3.80-4.00 (m, 1H), 7.47 (t, 1H, J=7.4 Hz), 7.56-7.63 (m, 2H), 7.77-7.84 (m, 2H), 8.00 (t, 1H, J=5.5 Hz), 8.37 (t, 1H, J=5.5 Hz), 9.03 (s, 1H); m/z (APCI pos) 410.2 (100%) [M+H-Boc].

Compounds of the following structures were prepared from the corresponding acids, using a similar method to that described above.

| Ex # | Structure | Name | Physical Data |
|---|---|---|---|
| E3 | 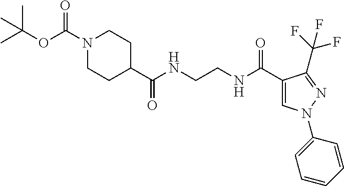 | tert-butyl 4-((2-(1- phenyl-3- (trifluoro- methyl)-1H- pyrazole-4- carboxamido) ethyl)carba- moyl)piperi- dine-1- carboxylate | White solid (72%); ¹H NMR (400 MHz, DMSO-d₆) δ 1.31-1.43 (m, 2H), 1.38 (s, 9H), 1.62- 1.69 (m, 2H), 2.20- 2.30m ( , 1H ), 2.70 (br, 2H), 3.18-3.30 (m, 4H), 3.80-4.00 (m, 2H), 7.48 (t, 1H, J = 7.4 Hz), 7.56-7.63 (m, 2H), 7.77-7.84 (m, 2H), 7.91 (t, 1H, J = 5.5 Hz), 8.35 (t, 1H, J = 5.5 Hz), 9.03 (s, 1H); m/z (APCI pos) 410.2 (100%) [M + H − Boc]. |
| E4 | 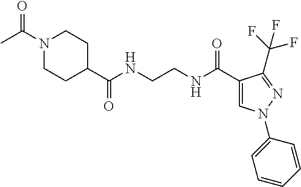 | 1-acetyl-N- (2-(1- phenyl-3- (trifluoro- methyl)-1H- pyrazole-4- carboxamido) ethyl)piperi- dine-4- carboxamide | White solid (80%); ¹H NMR (400 MHz, DMSO-d₆) δ 1.34 (m, 1H), 1.48 (m, 1H), 1.68 (m, 2H), 1.97 (s, 3H), 2.32 (m, 1H), 2.53 (m, 1H), 3.00 (m, 1H), 3.16- 3.32 (m, 4H), 3.78 (d, 1H, J = 13.7 Hz), 4.32 (d, 1H, J = 12.9 Hz), 7.48 (t, 1H, J = 7.4 Hz), 7.56-7.63 (m, 2H), 7.77-7.84 (m, 2H), 7.92 (t, 1H, J = 5.5 Hz), 8.36 (t, 1H, J = 5.5 Hz), 9.03 (s, 1H); m/z (APCI pos) 452.2 (100%) [M + H]. |
| E7 | 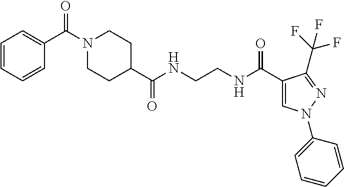 | 1-benzoyl-N- (2-(1- phenyl-3- (trifluoro- methyl)-1H- pyrazole-4- carboxamido) ethyl)piperi- dine-4- carboxamide | White solid (21%); ¹H NMR (400 MHz, CD₃OD) δ 1.52-1.94 (m, 4H), 2.49 (m, 1H), 2.89 (m, 1H), 3.11 (m, 1H), 3.36-3.50 (m, 4H), 3.72 (m, 1H), 4.61 (m, 1H), 7.34-7.38 (m, 2H), 7.40-7.48 (m, 4H), 7.54-7.59 (m, 2H), 7.80-7.84 (m, 2H), 8.71 (s, 1H); m/z (APCI pos) 514.2 (100%) [M + H]. |
| E168 | 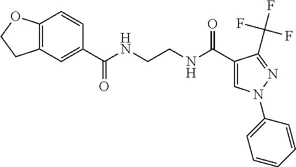 | N-(2-(2,3- dihydro- benzo- furan-5- carboxamido) ethyl)-1- phenyl-3- (trifluoro- methyl)-1H- pyrazole-4- carboxamide | ¹H NMR (400 MHz, CDCl₃) δ 3.21 (t, J = 8.8 Hz, 2H), 3.67 (s, 4H), 4.62 (t, J = 8.8 Hz, 2H), 6.78 (d, J = 8.4 Hz, 1H), 7.00 (br, 1H), 7.18 (br, 1H), 7.38-7.42 (m, 1H), 7.47-7.50 (m, 2H), 7.60-7.62 (m, 1H), 7.66-7.69 (m, 3H), 8.45 (br, 1H); m/z (APCI pos) Found: 445 (M + H). |

### Example E5

### N-(2-(1-Phenyl-3-(trifluoromethyl)-1H-pyrazole-4-carboxamido)ethyl)piperidine-4-carboxamide

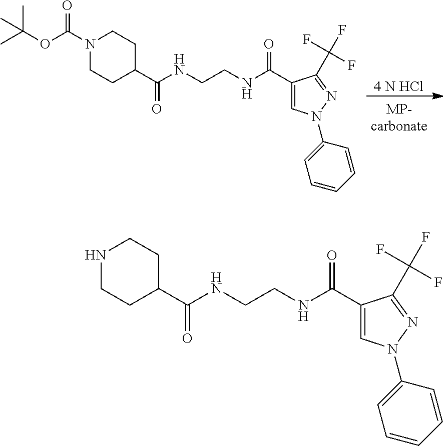

To tert-butyl 4-(2-(1-phenyl-3-(trifluoromethyl)-1H-pyrazole-4-carboxamido) ethylcarbamoyl)piperidine-1-carboxylate (500 mg, 0.998 mmol) in DCM (10 mL) was added 4N HCl in dioxane (3 mL). The mixture was stirred at room temperature for 1 hour then concentrated to dryness to isolate N-(2-(1-phenyl-3-(trifluoromethyl)-1H-pyrazole-4-carboxamido)ethyl)piperidine-4-carboxamide hydrochloride as a white solid (quant.).

The residue was dissolved in MeOH (10 mL) and MP-carbonate was added. After 1 hour, the mixture was filtered and the filtrate concentrated to yield N-(2-(1-phenyl-3-(trifluoromethyl)-1H-pyrazole-4-carboxamido)ethyl)piperidine-4-carboxamide as a white solid (400 mg, quant): ¹H NMR (400 MHz, DMSO-d₆) δ 1.65-1.90 (m, 4H), 2.40 (m, 1H), 2.82-2.94 (m, 2H), 3.18-3.32 (m, 5H), 7.48 (t, 1H, J=7.4 Hz), 7.54-7.63 (m, 2H), 7.80-7.84 (m, 2H), 8.09 (t, 1H, J=5.5 Hz), 8.37 (bd, 1H, J=9.2 Hz), 8.45 (t, 1H, J=5.5 Hz), 8.69 (bd, 1H, J=9.2 Hz), 9.12 (s, 1H); m/z (APCI pos) 410.2 (100%) [M+H].

Compounds of the following structures were prepared as the free base or the HCl salt from the corresponding Boc-protected amines, using a similar method to that described above.

| Ex # | Structure | Name | Physical Data |
|---|---|---|---|
| E8 | 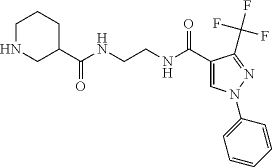 | N-(2-(1- phenyl-3- (trifluoro- methyl)-1H- pyrazole-4- carboxamido) ethyl)piperi- dine-3- carboxamide | White solid (quant.); ¹H NMR (400 MHz, DMSO- d₆) δ 1.30-1.60 (m, 3H), 1.72-1.81 (m, 1H), 2.20-2.30 (m, 1H), 2.44-2.50 (m, 1H), 2.59 (m, 1H), 2.84 (m, 1H), 2.96 (m, 1H), 3.18-3.32 (m, 4H), 3.80 (br, 1H), 7.48 (t, 1H, J = 7.4 Hz), 7.54-7.63 (m, 2H), 7.80-7.84 (m, 2H), 8.00 (t, 1H, J = 5.5 Hz), 8.40 (t, 1H, J = 5.5 Hz), 9.07 (s, 1H); m/z (APCI pos) 410.2 (100%) [M + H]. |
| E237 | 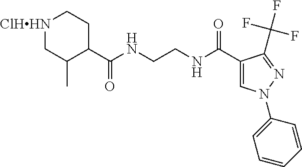 | 3-methyl-N- (2-(1- phenyl-3- (trifluoro- methyl)-1H- pyrazole-4- carboxamido) ethyl)piperi- dine-4- carboxamide hydrochloride | White solid (quant.); ¹H NMR (400 MHz, DMSO- d₆) δ 0.88 (d, 3H, J = 7.0 Hz), 1.78-1.87 (m, 2H), 2.10-2.17 (m, 1H), 2.86-3.45 (m, 9H), 7.45-7.50 (m, 1H), 7.58-7.64 (m, 2H), 7.82-7.85 (m, 2H), 8.30 (t, 1H, J = 5.9 Hz), 8.57 (t, 1H, J = 5.9 Hz), 8.70 (br, 1H), 8.82 (br, 1H), 9.34 (s, 1H); m/z (APCI pos) 424.1 (100%) [M + H]. |
| E238 | 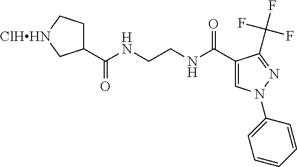 | 1-phenyl-N- (2- (pyrrolidine- 3- carboxamido) ethyl)-3- (trifluoro- methyl)-1H- pyrazole-4- carboxamide hydrochloride | White solid (quant.); ¹H NMR (400 MHz, DMSO- d₆) δ 1.90-2.19 (m, 2H), 3.00-3.34 (m, 9H), 7.45-7.50 (m, 1H), 7.58-7.64 (m, 2H), 7.82-7.85 (m, 2H), 8.46 (t, 1H, J = 5.9 Hz), 8.60 (t, 1H, J = 5.9 Hz), 9.00 (br, 1H), 9.12 (br, 1H), 9.33 (s, 1H); m/z (APCI pos) 396.2 (100%) [M + H]. |
| E239 | 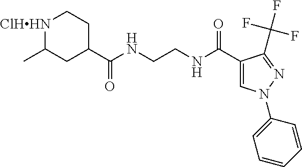 | 2-methyl-N- (2-(1- phenyl-3- (trifluoro- methyl)-1H- pyrazole-4- carboxamido) ethyl)piperi- dine-4- carboxamide hydrochloride | Solid (100%); ¹H NMR (400 MHz, DMSO-d₆) δ 1.20 (d, 3H, J = 6.6 Hz), 1.47 (m, 0.5H), 1.66 (m, 0.5H), 1.87 (m, 2H), 2.43 (m, 1H), 2.84 (m, 1H), 3.11 (m, 1H), 3.25 (m, 6H), 7.48 (m, 1H), 7.61 (m, 2H), 7.82 (m, 2H), 8.14 (m, 1H), 8.51 (m, 1H), 9.23 (s, 1H); m/z (APCI pos) 424.2 (100%) [M + H]. |
| E240 | 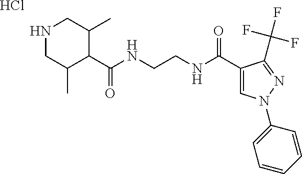 | 2,6- dimethyl-N- (2-(1- phenyl-3- (trifluoro- methyl)-1H- pyrazole-4- carboxamido) ethyl)pipera- zine-1- carboxamide hydrochloride | m/z (APCI pos) 438.9 (50%) [M + H]. |
| E241 | 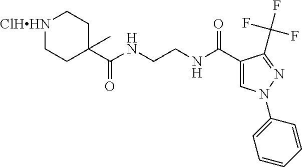 | 4-methyl-N- (2-(1- phenyl-3- (trifluoro- methyl)-1H- pyrazole-4- carboxamido) ethyl)piperi- dine-4- carboxamide hydrochloride | White Solid (100%); ¹H NMR (400 MHz, DMSO-d₆) δ .m, 3H), 1.50-1.60 (m, 2H), 2.13-2.20 (m, 2H), 2.80-2.90 (m, 2H), 3.08-3.14 (m, 2H), 3.25-3.34 (m, 3H), 7.45-7.50 (m, 1H), 7.58-7.64 (m, 2H), 7.82-7.85 (m, 2H), 8.13 (t, 1H, J = 5.9 Hz), 8.60-8.65 (m, 3H), 9.00 (br, 1H), 9.12 (br, 1H), 9.39 (s, 1H). |

### Example E6

### N-(2-(1-Phenyl-3-(trifluoromethyl)-1H-pyrazole-4-carboxamido)ethyl)-1-propylpiperidine-3-carboxamide

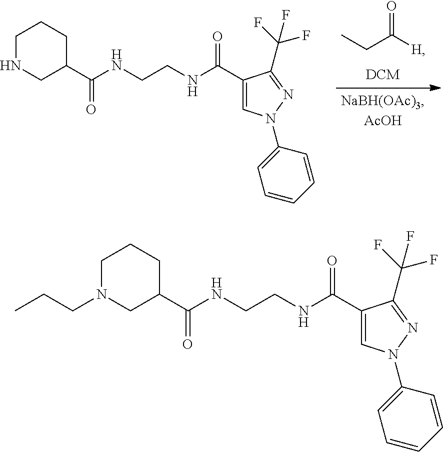

To N-(2-(1-phenyl-3-(trifluoromethyl)-1H-pyrazole-4-carboxamido)ethyl)piperidine-3-carboxamide (50 mg, 0.12 mmol) in DCM (3 mL) was added propionaldehyde (9.2 mg, 0.16 mmol). The mixture was stirred for 2 hours at room temperature then sodium triacetoxyborohydride was added (44 mg, 0.21 mmol). The pH of the mixture was lowered by addition of acetic acid and the mixture was stirred overnight. The mixture was made neutral with addition of 2N NaOH then the aqueous layer was extracted with DCM. The combined organic layers were dried over MgSO₄ then concentrated. Purification by MPLC (DCM/MeOH/NH₄OH 90/10/0.1) yielded N-(2-(1-phenyl-3-(trifluoromethyl)-1H-pyrazole-4-carboxamido)ethyl)-1-propylpiperidine-3-carboxamide as a white powder (35 mg, 63%): ¹H NMR (400 MHz, CD₃OD) δ 0.88 (t, 3H, J=7.4 Hz), 1.48-1.88 (m, 5H), 2.20-2.55 (m, 4H), 2.84-3.00 (m, 2H), 3.28-3.34 (m, 4H), 3.36-3.52 (m, 4H), 7.45 (t, 1H, J=7.4 Hz), 7.54-7.61 (m, 2H), 7.80-7.84 (m, 2H), 8.74 (s, 1H); m/z (APCI pos) 452.3 (100%) [M+H].

### Example E9

### tert-Butyl 4-(2-(1-phenyl-3-(trifluoromethyl)-1H-pyrazole-4-carboxamido)ethylcarbamoyl)piperazine-1-carboxylate

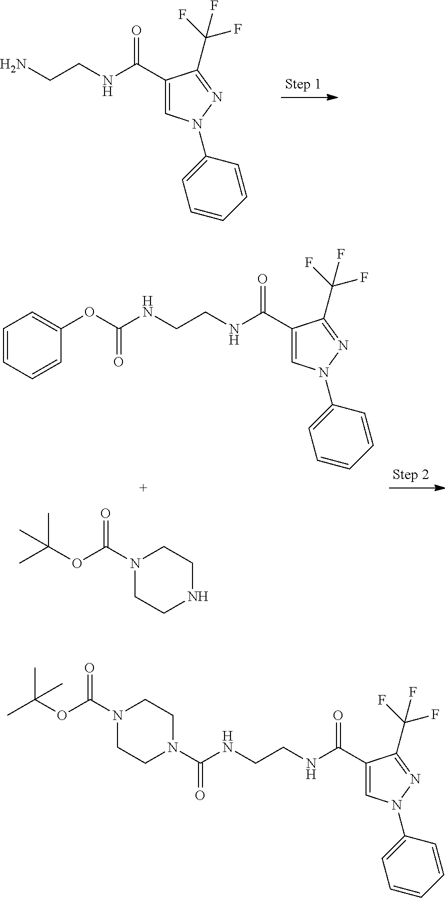

### Step 1

To N-(2-aminoethyl)-1-phenyl-3-(trifluoromethyl)-1H-pyrazole-4-carboxamide (7 g, 23 mmol) and triethylamine (4.7 g, 47 mmol) in DCM (200 mL) at 0° C., was added dropwise phenyl chloroformate (4.4 g, 28 mmol) in DCM (50 mL). The mixture was stirred at room temperature for 2 hours then concentrated to dryness. The residue was purified by MPLC (DCM/MeOH 99/1) to yield phenyl 2-(1-phenyl-3-(trifluoromethyl)-1H-pyrazole-4-carboxamido)ethylcarbamate as a white solid (8.2 g, 84%). Crystallized from AcOEt/hexanes: ¹H NMR (400 MHz, DMSO-d₆) δ 3.20-3.28 (m, 2H), 3.34-3.42 (m, 2H), 7.10 (m, 2H), 7.19 (m, 1H), 7.36 (m, 2H), 7.48 (m, 1H), 7.61 (m, 2H), 7.80 (m, 2H), 7.85 (t, 1H, J=5.9 Hz), 8.47 (t, 1H, J=5.9 Hz), 9.04 (s, 1H); m/e (APCI pos) 419.0 (20%), 299.0 (100%) [M+H].

### Step 2

To phenyl 2-(1-phenyl-3-(trifluoromethyl)-1H-pyrazole-4-carboxamido)ethylcarbamate (200 mg, 0.48 mmol) was added Boc-piperazine in EtOH (1 mL) and the mixture was heated to 150° C. for 10 minutes in an open vessel. The crude mixture was purified by MPLC (DCM/MeOH 100/0->95/5) to yield tert-butyl 4-(2-(1-phenyl-3-(trifluoromethyl)-1H-pyrazole-4-carboxamido)ethylcarbamoyl)piperazine-1-carboxylate as a white solid (156 mg, 64%): ¹H NMR (400 MHz, DMSO-d₆) δ 1.40 (s, 9H), 3.15-3.35 (m, 12H), 6.72 (t, 1H, J=5.5 Hz), 7.48 (m, 1H), 7.61 (m, 2H), 7.81 (m, 2H), 8.40 (t, 1H, J=5.5 Hz), 9.06 (s, 1H); m/z (APCI pos) 411.0 (40%) [M+H-Boc].

Compounds of the following structures were prepared from the corresponding amines, using a similar method to that described above with reaction temperatures ranging from 120° C. to 170° C. and reaction times ranging from 10 minutes to 2 hours.

| Ex# | Structure | Name | Physical Data |
|---|---|---|---|
| E11 | 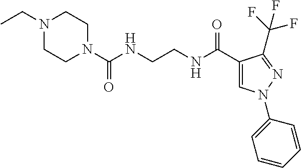 | 4-ethyl-N-(2- (1-phenyl-3- (trifluoro- methyl)-1H- pyrazole-4- carboxamido) ethyl)pipera- zine-1- carboxamide | White solid (62%); ¹H NMR (400 MHz, DMSO-d₆) δ 0.98 (t, 3H, J = 7.0 Hz), 2.24-2.33 (m, 6H), 3.14-3.21 (m, 2H), 3.24-3.32 (m, 6H), 6.64 (t, 1H, J = 5.5 Hz), 7.48 (m, 1H), 7.61 (m, 2H), 7.82 (m, 2H), 8.39 (t, 1H, J = 5.5 Hz), 9.05 (s, 1H); m/z (APCI pos) 439.1 (100%) [M + H]. |
| E13 | 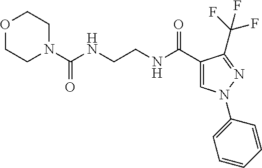 | N-(2-(1- phenyl-3- (trifluoro- methyl)-1H- pyrazole-4- carboxamido) ethyl) morpholine-4- carboxamide | White solid (66%); ¹H NMR (400 MHz, DMSO-d₆) δ 3.16-3.32 (m, 8H), 3.50-3.55 (m, 4H), 6.69 (t, 1H, J = 5.5 Hz), 7.48 (m, 1H), 7.61 (m, 2H), 7.82 (m, 2H), 8.40 (t, 1H, J = 5.5 Hz), 9.06 (s, 1H); m/z (APCI pos) 412.1 (40%) [M + H]. |
| E14 | 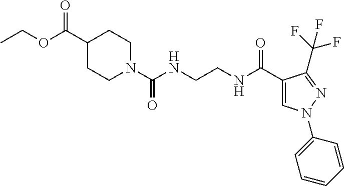 | Ethyl 1-((2- (1-phenyl-3- (trifluoro- methyl)-1H- pyrazole-4- carboxamido) ethyl) carbamoyl) piperidine- 4-carboxylate | White solid (51%),; ¹H NMR (400 MHz, DMSO-d₆) δ 1.17 (t, 3H, J = 7.0 Hz), 1.39 (m, 2H), 1.75 (m, 2H), 2.42- 2.50 (m, 1H), 2.77 (m, 2H), 3.18 (m, 2H), 3.28 (m, 2H), 3.85 (m, 2H), 4.05 (q, 2H, J = 7.0 Hz), 6.63 (t, 1H, J = 5.5 Hz), 7.48 (m, 1H), 7.61 (m, 2H), 7.82 (m, 2H), 8.39 (t, 1H, J = 5.5 Hz), 9.05 (s, 1H); m/z (APCI pos) 482.1 (50%) [M + H]. |
| E15 | 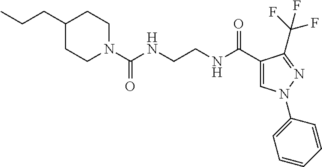 | N-(2-(1- phenyl-3- (trifluoro- methyl)-1H- pyrazole-4- carboxamido) ethyl)-4- propyl- piperidine-1- carboxamide | White solid (63%); ¹H NMR (400 MHz, DMSO-d₆) δ 0.84 (t, 3H, J = 7.4 Hz), 0.88-0.96 (m, 2H), 1.10-1.40 (m, 5H), 1.56 (m, 2H), 2.60 (m, 2H), 3.17 (m, 2H), 3.27 (m, 2H), 2.92 (m, 2H), 6.56 (t, 1H, J = 5.5 Hz), 7.48 (m, 1H), 7.61 (m, 2H), 7.82 (m, 2H), 8.37 (t, 1H, J = 5.5 Hz), 9.05 (s, 1H); m/z (APCI pos) 452.1 (50%) [M + H]. |
| E16 | 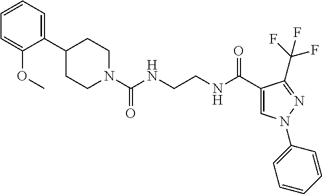 | 4-(2- methoxyphenyl)- N-(2-(1- phenyl-3- (trifluoro- methyl)-1H- pyrazole-4- carboxamido) ethyl) piperidine-1- carboxamide | White solid (46%); ¹H NMR (400 MHz, DMSO-d₆) δ 1.44 (m, 2H), 1.64 (m, 2H), 2.74 (m, 2H), 3.03 (m, 1H), 3.21 (m, 2H), 3.31 (m, 2H), 3.78 (s, 3H), 4.08 (m, 2H), 6.65 (t, 1H, J = 5.5 Hz), 6.87 (m, 1H), 6.92 (m, 1H), 7.10 (m, 1H), 7.17 (m, 1H), 7.47 (m, 1H), 7.60 (m, 2H), 7.82 (m, 2H), 8.40 (t, 1H, J = 5.5 Hz), 9.06 (s, 1H); m/z (APCI pos) 516.1 (60%) [M + H]. |
| E17 | 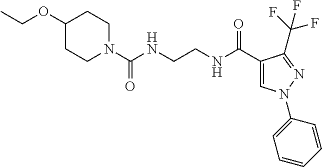 | 4-ethoxy-N- (2-(1-phenyl- 3- (trifluoro- methyl)-1H- pyrazole-4- carboxamido) ethyl) piperidine-1- carboxamide | White solid (80%); ¹H NMR (400 MHz, DMSO-d₆) δ 1.09 (t, 3H, J = 7.0 Hz), 1.26 (m, 2H), 1.76 (m, 2H), 2.92 (m, 2H), 3.17 (m, 2H), 3.29 (m, 2H), 3.40 (m, 1H), 3.44 (q, 2H, J = 7.0 Hz), 3.65 (m, 2H), 6.62 (t, 1H, J = 5.5 Hz), 7.48 (m, 1H), 7.61 (m, 2H), 7.82 (m, 2H), 8.39 (t, 1H, J = 5.5 Hz), 9.05 (s, 1H); m/z (APCI pos) 454.1 (80%) [M + H]. |
| E18 | 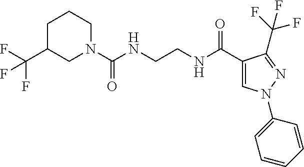 | N-(2-(1- phenyl-3- (trifluoro- methyl)-1H- pyrazole-4- carboxamido) ethyl)-3- (trifluoro- methyl) piperidine-1- carboxamide | White solid (81%); ¹H NMR (400 MHz, DMSO-d₆) δ 1.30-1.50 (m, 2H), 1.60-1.68 (m, 1H), 1.87-1.95 (m, 1H), 2.24-2.36 (m, 1H), 2.60-2.75 (m, 2H), 3.15-3.22 (m, 2H), 3.27-3.34 (m, 2H), 3.82-3.89 (m, 1H), 4.14-4.20 (m, 1H), 6.78 (t, 1H, J = 5.5 Hz), 7.48 (m, 1H), 7.61 (m, 2H), 7.81 (m, 2H), 8.38 (t, 1H, J = 5.5 Hz), 9.05 (s, 1H); m/z (APCI pos) 478.1 (30%) [M + H]. |
| E19 | 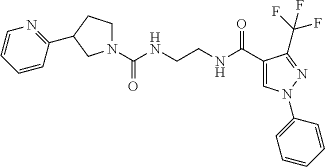 | 1-phenyl-N- (2-(3- (pyridin-2- yl) pyrrolidine-1- carboxamido) ethyl)-3- (trifluoro- methyl)-1H- pyrazole-4- carboxamide | Tan solid (67%); ¹H NMR (400 MHz, DMSO-d₆) δ 2.00-2.11 (m, 1H), 2.18-2.26 (m, 1H), 3.16-3.22 (m, 2H), 3.26-3.42 (m, 4H), 3.45-3.57 (m, 2H), 3.71 (dd, 1H, J = 9.4, 7.8 Hz), 6.51 (t, 1H, J = 5.5 Hz), 7.22-7.26 (m, 1H), 7.31-7.34 (m, 1H), 7.48 (m, 1H), 7.61 (m, 2H), 7.73 (dt, 1H, J = 7.8, 1.9 Hz), 7.81 (m, 2H), 8.42 (t, 1H, J = 5.5 Hz), 8.51 (m, 1H), 9.06 (s, 1H); m/z (APCI pos) 473.1 (20%) [M + H]. |
| E20 | 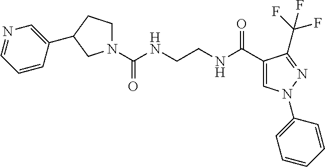 | 1-phenyl-N- (2-(3- (pyridin-3- yl) pyrrolidine-1- carboxamido) ethyl)-3- (trifluoro- methyl)-1H- pyrazole-4- carboxamide | White solid (76%); ¹H NMR (400 MHz, DMSO-d₆) δ 1.90-2.02 (m, 1H), 2.20-2.30 (m, 1H), 3.16-3.24 (m, 3H), 3.26-3.32 (m, 3H), 3.35-3.44 (m, 1H), 3.46-3.52 (m, 1H), 3.75 (dd, 1H, J = 9.8, 7.4 Hz), 6.35 (t, 1H, J = 5.5 Hz), 7.32-7.36 (m, 1H), 7.45-7.50 (m, 1H), 7.60 (m, 2H), 7.73 (dt, 1H, J = 7.8, 2.3 Hz), 7.82 (m, 2H), 8.42 (t, 1H, J = 5.5 Hz), 8.44 (dd, 1H, J = 5.0, 1.6 Hz), 8.51 (d, 1H, J = 2.3 Hz), 9.06 (d, 1H, J = 1.7 Hz); m/z (APCI pos) 473.2 (100%) [M + H]. |
| E21 | 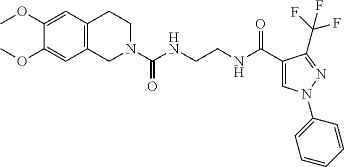 | 6,7- dimethoxy-N- (2-(1-phenyl- 3- (trifluoro- methyl)-1H- pyrazole-4- carboxamido) ethyl)-3,4- dihydroisoqui- noline-2(1H)- carboxamide | White solid (75%); ¹H NMR (400 MHz, DMSO-d₆) δ 2.66 (t, 2H, J = 5.8 Hz), 3.18-3.24 (m, 2H), 3.28-3.34 (m, 2H), 3.51 (t, 2H, J = 5.8 Hz), 3.68 (s, 3H), 3.70 (s, 3H), 4.40 (s, 2H), 6.65-6.72 (m, 3H), 7.45-7.50 (m, 1H), 7.60 (m, 2H), 7.80 (m, 2H), 8.42 (t, 1H, J = 5.5 Hz), 9.04 (d, 1H, J = 1.7 Hz); m/z (APCI pos) 518.0 (100%) [M + H]. |
| E22 | 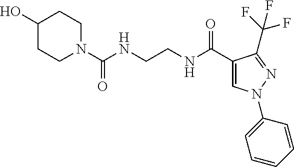 | 4-hydroxy-N- (2-(1-phenyl- 3- (trifluoro- methyl)-1H- pyrazole-4- carboxamido) ethyl) piperidine-1- carboxamide | White solid (78%); ¹H NMR (400 MHz, DMSO-d₆) δ 1.17-1.27 (m, 2H), 1.61-1.70 (m, 2H), 2.88 (ddd, 2H, J = 13.3, 10.1, 3.1, 2.7 Hz), 3.14-3.20 (m, 2H), 3.25-3.31 (m, 2H), 3.55-3.71 (m, 3H), 4.64 (d, 1H, J = 4.3 Hz), 6.60 (t, 1H, J = 5.5 Hz), 7.48 (m, 1H), 7.60 (m, 2H), 7.82 (m, 2H), 8.40 (t, 1H, J = 5.5 Hz), 9.05 (s, 1H); m/z (APCI pos) 426.1 (40%) [M + H]. |
| E23 | 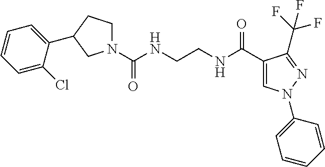 | N-(2-(3-(2- chlorophenyl) pyrrolidine- 1- carboxamido) ethyl)-1- phenyl-3- (trifluoro- methyl)-1H- pyrazole-4- carboxamide | White solid (79%); ¹H NMR (400 MHz, DMSO-d₆) δ 1.95-2.05 (m, 1H), 2.15-2.25 (m, 1H), 3.16-3.25 (m, 3H), 3.28-3.38 (m, 3H), 3.40-3.46 (m, 1H), 3.65-3.75 (m, 2H), 6.36 (t, 1H, J = 5.5 Hz), 7.24-7.35 (m, 2H), 7.37-7.40 (m, 1H), 7.43-7.50 (m, 2H), 7.60 (m, 2H), 7.82 (m, 2H), 8.41 (t, 1H, J = 5.5 Hz), 9.06 (s, 1H); m/z (APCI pos) 506.1 (100%) [M + H]. |
| E24 | 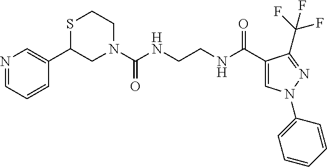 | N-(2-(1- phenyl-3- (trifluoro- methyl)-1H- pyrazole-4- carboxamido) ethyl)-2- (pyridin-3- yl)thiomorpho- line-4- carboxamide | White solid (61%); ¹H NMR (400 MHz, DMSO-d₆) δ 2.60 (m, 1H), 2.81 (m, 1H), 3.04 (m, 1H), 3.22 (m, 2H), 3.30 (m, 3H), 3.96 (m, 1H), 4.29 (m, 2H), 6.82 (m, 1H), 7.34 (m, 1H), 7.48 (m, 1H), 7.60 (m, 2H), 7.79 (m, 3H), 8.39 (m, 1H), 8.48 (m, 1H), 8.57 (m, 1H), 9.05 (s, 1H); m/z (APCI pos) 505.2 (100%) [M + H]. |
| E25 | 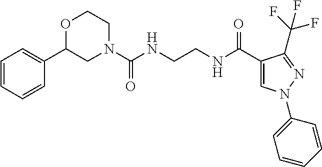 | 2-phenyl-N- (2-(1-phenyl- 3- (trifluoro- methyl)-1H- pyrazole-4- carboxamido) ethyl) morpholine-4- carboxamide | White solid (23%); ¹H NMR (400 MHz, DMSO-d₆) δ 2.85 (m, 1H), 3.09 (m, 1H), 3.44-3.59 (m, 4H), 3.65-3.79 (m, 2H), 3.96-4.08 (m, 2H), 4.41 (m, 1H), 5.57 (m, 1H), 7.27- 7.49 (m, 9H), 7.67 (m, 2H), 8.43 (s, 1H); m/z (APCI pos) 488.1 (100%) [M + H]. |
| E26 | 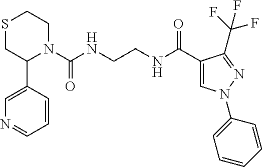 | N-(2-(1- phenyl-3- (trifluoro- methyl)-1H- pyrazole-4- carboxamido) ethyl)-3- (pyridin-3- yl)thiomorpho- line-4- carboxamide | White solid (43%); ¹H NMR (400 MHz, DMSO-d₆) δ 2.40 (m, 1H), 2.71 (m, 1H), 2.92 (m, 1H), 3.08 (m, 1H), 3.22- 3.35 (m, 5H), 4.08 (m, 1H), 5.66 (m, 1H), 6.87 (m, 1H), 7.34 (m, 1H), 7.48 (m, 1H), 7.60 (m, 3H), 7.79 (m, 2H), 8.41 (m, 2H), 8.49 (m, 1H), 9.02 (s, 1H); m/z (APCI pos) 505.1 (100%) [M + H]. |
| E29 | 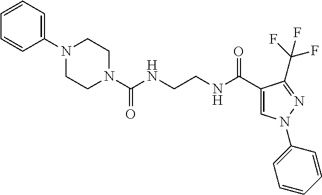 | 4-phenyl-N- (2-(1-phenyl- 3- (trifluoro- methyl)-1H- pyrazole-4- carboxamido) ethyl) piperazine-1- carboxamide | White solid (47%); ¹H NMR (400 MHz, DMSO-d₆) δ 3.10 (m, 4H), 3.21 (m, 2H), 3.31 (m, 2H), 3.45 (m, 4H), 6.77- 6.84 (m, 2H), 6.98 (m, 2H), 7.22 (m, 2H), 7.48 (m, 1H), 7.60 (m, 2H), 7.81 (m, 2H), 8.43 (t, 1H, J = 5.5 Hz), 9.08 (s, 1H); m/z (APCI pos) 487.1 (40%) [M + H]. |
| E30 | 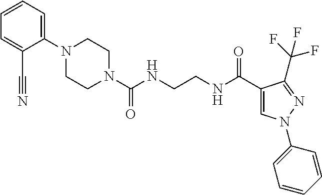 | 4-(2- cyanophenyl)- N-(2-(1- phenyl-3- (trifluoro- methyl)-1H- pyrazole-4- carboxamide) ethyl) piperazine-1- carboxamide | White solid (77%); ¹H NMR (400 MHz, DMSO-d₆) δ 3.06-3.12 (m, 4H), 3.18-3.24 (m, 2H), 3.28-3.34 (m, 2H), 3.46-3.52 (m, 4H), 6.69 (t, 1H, J = 5.5 Hz), 7.08-7.18 (m, 2H), 7.48 (m, 1H), 7.58-7.64 (m, 3H), 7.70-7.74 (m, 1H), 7.80-7.84 (m, 2H), 8.42 (t, 1H, J = 5.5 Hz), 9.07 (s, 1H); m/z (APCI pos) 512.1 (100%) [M + H]. |
| E31 | 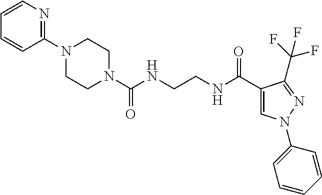 | N-(2-(1- phenyl-3- (trifluoro- methyl)-1H- pyrazole-4- carboxamido) ethyl)-4- (pyridin-2- yl)piperazine- 1- carboxamide | White solid (77%); ¹H NMR (400 MHz, DMSO-d₆) δ 3.18-3.24 (m, 2H), 3.28-3.34 (m, 2H), 3.38-3.48 (m, 8H), 6.62-6.67 (m, 1H), 6.76 (t, 1H, J = 5.5 Hz), 6.82-6.86 (m, 1H), 7.44-7.65 (m, 4H), 7.78-7.84 (m, 2H), 8.10-8.13 (m, 1H), 8.41 (t, 1H, J = 5.5 Hz), 9.06 (s, 1H); m/z (APCI pos) 488.1 (100%) [M + H]. |
| E32 | 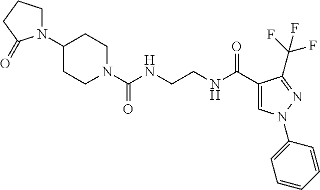 | 4-(2- oxopyrrolidin- 1-yl)-N-(2- (1-phenyl-3- (trifluoro- methyl)-1H- pyrazole-4- carboxamido) ethyl) piperidine-1- carboxamide | Beige solid (66%); ¹H NMR (400 MHz, DMSO-d₆) δ 1.40-1.50 (m, 4H), 1.80-1.90 (m, 2H), 2.19 (t, 2H, J = 8.2 Hz), 2.65-2.74 (m, 2H), 3.16-3.24 (m, 4H), 3.26-3.32 (m, 2H), 3.88 (m, 1H), 4.00-4.06 (m, 2H), 6.67 (t, 1H, J = 5.5 Hz), 7.45-7.50 (m, 1H), 7.58-7.64 (m, 2H), 7.78-7.83 (m, 2H), 8.38 (t, 1H, J = 5.5 Hz), 9.06 (s, 1H); m/z (APCI pos) 493.1 (100%) [M + H]. |
| E33 | 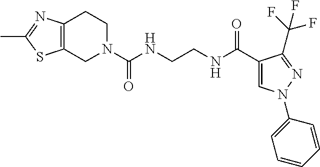 | 2-methyl-N- (2-(1-phenyl- 3- (trifluoro- methyl)-1H- pyrazole-4- carboxamido) ethyl)-6,7- dihydro- thiazolo[5,4- c]pyridine- 5(4H)- carboxamide | Beige solid (37%); ¹H NMR (400 MHz, DMSO-d₆) δ 2.57 (s, 3H), 2.65- 2.70 (m, 2H), 3.18- 3.24 (m, 2H), 3.27- 3.35 (m, 2H), 3.63 (t, 2H, J = 8.9 Hz), 4.52 (s, 2H), 6.86 (t, 1H, J = 5.5 Hz), 7.45-7.50 (m, 1H), 7.58-7.64 (m, 2H), 7.78-7.83 (m, 2H), 8.39 (t, 1H, J = 5.5 Hz), 9.03 (s, 1H); m/z (APCI pos) 479.0 (100%) [M + H]. |
| E35 | 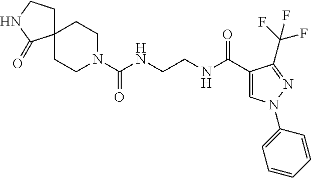 | 4-spiro-[3- (2- pyrrolidinone)]- N-(2-(1- phenyl-3- (trifluoro- methyl)-1H- pyrazole-4- carboxamido) ethyl) piperidine-1- carboxamide | Beige solid (26%); ¹H NMR (400 MHz, DMSO-d₆) δ 1.24-1.30 (m, 2H), 1.48-1.56 (m, 2H), 1.95 (d, 2H, J = 7.0 Hz), 2.80-2.88 (m, 2H), 3.14-3.22 (m, 4H), 3.26-3.32 (m, 2H), 3.83 (m, 2H), 6.64 (t, 1H, J = 5.5 Hz), 7.45-7.50 (m, 1H), 7.54-7.64 (m, 3H), 7.79-7.83 (m, 2H), 8.41 (t, 1H, J = 5.5 Hz), 9.06 (s, 1H); m/z (APCI pos) 479.1 (80%) [M + H]. |
| E36 | 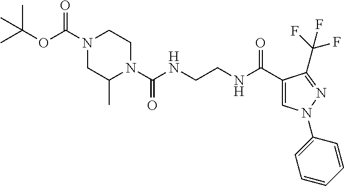 | tert-butyl 3- methyl-4-((2- (1-phenyl-3- (trifluoro- methyl)-1H- pyrazole-4- carboxamido) ethyl) carbamoyl) piperazine-1- carboxylate | White solid (64%); ¹H NMR (400 MHz, DMSO-d₆) δ 0.98 (d, 3H, J = 6.64 Hz), 1.40 (s, 9H), 2.60-3.05 (bm, 3H), 3.16-3.33 (m, 4H), 3.67 (m, 2H), 3.75-4.16 (bm, 2H), 6.64 (t, 1H, J = 5.5 Hz), 7.45-7.50 (m, 1H), 7.58-7.64 (m, 2H), 7.79-7.83 (m, 2H), 8.39 (t, 1H, J = 5.5 Hz), 9.05 (s, 1H); m/z (APCI pos) 469.1 (100%) [M − tBu), 525.1 (5%) [M + H]. |
| E37 | 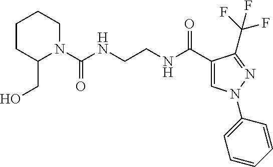 | 2- (hydroxymethyl)- N-(2-(1- phenyl-3- (trifluoro- methyl)-1H- pyrazole-4- carboxamido) ethyl) piperidine-1- carboxamide | Beige solid (43%); ¹H NMR (400 MHz, DMSO-d₆) δ 1.20-1.60 (m, 5H), 1.71-1.77 (m, 1H), 2.70 (dt, 1H), 3.15- 3.21 (m, 2H), 3.25- 3.30 (m, 2H), 3.46 (m, 2H), 3.80 (bd, 1H), 4.03 (m, 1H), 4.65 (t, 1H, J = 5.5 Hz), 6.43 (t, 1H, J = 5.5 Hz), 7.45-7.50 (m, 1H), 7.58-7.64 (m, 2H), 7.80-7.84 (m, 2H), 8.36 (t, 1H, J = 5.5 Hz), 9.04 (s, 1H); m/z (APCI pos) 440.1 (100%) [M + H]. |
| E38 | 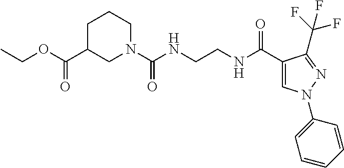 | ethyl 1-((2- (1-phenyl-3- (trifluoro- methyl)-1H- pyrazole-4- carboxamido) ethyl) carbamoyl) piperidine-3- carboxylate | Beige solid (74%); ¹H NMR (400 MHz, DMSO-d₆) δ 1.16 (t, 3H, J = 7.0 Hz), 1.30-1.60 (m, 3H), 1.91 (m, 1H), 2.34 (m, 1H), 2.70- 2.86 (m, 2H), 3.14- 3.20 (m, 2H), 3.26- 3.32 (m, 2H), 3.75 (bd, 1H), 3.99-4.04 (m, 1H), 4.04 (q, 2H, J = 7.0 Hz), 6.67 (t, 1H, J = 5.5 Hz), 7.45- 7.50 (m, 1H), 7.58- 7.64 (m, 2H), 7.80- 7.84 (m, 2H), 8.37 (t, 1H, J = 5.5 Hz), 9.05 (s, 1H); m/z (APCI pos) 482.0 (100%) [M + H]. |
| E49 | 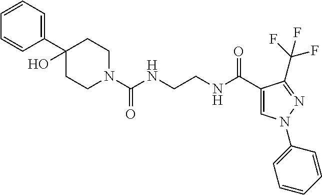 | 4-hydroxy-4- phenyl-N-(2- (1-phenyl-3- (trifluoro- methyl)-1H- pyrazole-4- carboxamido) ethyl) piperidine-1- carboxamide | White solid (73%); ¹H NMR (400 MHz, DMSO-d₆) δ 1.50-1.56 (m, 2H), 1.72-1.80 (m, 2H), 3.05-3.14 (m, 2H), 3.18-3.24 (m, 2H), 3.28-3.34 (m, 2H), 3.82-3.88 (m, 2H), 4.98 (s, 1H), 6.64 (t, 1H, J = 5.5 Hz), 7.18 (m, 1H), 7.26-7.32 (m, 2H), 7.40-7.50 (m, 3H), 7.60 (m, 2H), 7.80 (m, 2H), 8.40 (t, 1H, J = 5.5 Hz), 9.06 (s, 1H); m/z (APCI pos) 484.1 (100%) [M − OH]. |
| E50 | 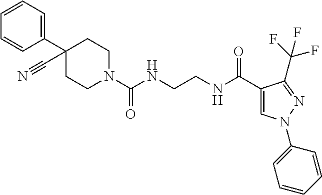 | 4-cyano-4- phenyl-N-(2- (1-phenyl-3- (trifluoro- methyl)-1H- pyrazole-4- carboxamido) ethyl) piperidine-1- carboxamide | White solid (80%); ¹H NMR (400 MHz, DMSO-d₆) δ 1.86-1.94 (m, 2H), 2.07-2.13 (m, 2H), 2.94-3.03 (m, 2H), 3.18-3.24 (m, 2H), 3.28-3.34 (m, 2H), 4.13-4.20 (m, 2H), 6.82 (t, 1H, J = 5.5 Hz), 7.34-7.53 (m, 6H), 7.57-7.62 (m, 2H), 7.78.7.84 (m, 2H), 8.40 (t, 1H, J = 5.5 Hz), 9.06 (s, 1H); m/z (APCI pos) 511.1 (100%) [M + H]. |
| E54 | 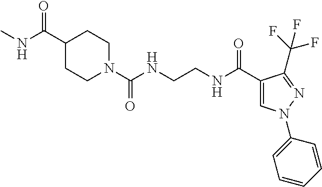 | N4-methyl-N1- (2-(1-phenyl- 3- (trifluoro- methyl)-1H- pyrazole-4- carboxamido) ethyl) piperidine-1,4- dicarboxamide | White solid (63%); ¹H NMR (400 MHz, DMSO-d₆) δ 1.33-1.45 (m, 2H), 1.56-1.64 (m, 2H), 2.18-2.27 (m, 1H), 2.54 (d, 3H, J = 4.6 Hz), 2.60-2.69 (m, 2H), 3.14-3.20 (m, 2H), 3.25-3.32 (m, 2H), 3.90-3.97 (m, 2H), 6.62 (t, 1H, J = 5.3 Hz), 7.48 (m, 1H), 7.58-7.63 (m, 2H), 7.67-7.72 (m, 1H), 7.78-7.82 (m, 2H), 8.41 (t, 1H, J = 5.5 Hz), 9.05 (s, 1H); m/z (APCI pos) 467.1 (100%) [M + H]. |
| E55 | 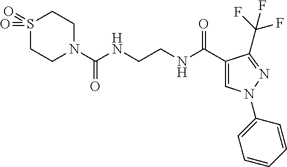 | N-(2-(1- phenyl-3- (trifluoro- methyl)-1H- pyrazole-4- carboxamido) ethyl)(1,1- dioxidothio- morpholin-4- yl)-4- carboxamide | White solid (26%); ¹H NMR (400 MHz, DMSO-d₆) δ 3.03-3.08 (m, 4H), 3.15-3.22 (m, 2H), 3.27-3.32 (m, 2H), 3.73-3.78 (m, 4H), 7.03 (t, 1H, J = 5.3 Hz), 7.48 (m, 1H), 7.58-7.63 (m, 2H), 7.78-7.82 (m, 2H), 8.42 (t, 1H, J = 5.5 Hz), 9.04 (s, 1H); m/z (APCI pos) 460.0 (50%) [M + H]. |
| E56 | 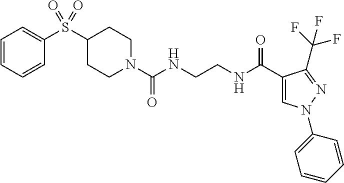 | N-(2-(1- phenyl-3- (trifluoro- methyl)-1H- pyrazole-4- carboxamido) ethyl)-4- (phenyl- sulfonyl) piperidine-1- carboxamide | White solid (26%); ¹H NMR (400 MHz, DMSO-d₆) δ 1.28-1.40 (rn, 2H), 1.73-1.78 (m, 2H), 2.62-2.70 (m, 2H), 3.12-3.18 (m, 2H), 3.23-3.28 (m, 2H), 3.42-3.52 (m, 1H), 3.98-4.06 (m, 2H), 6.71 (t, 1H, J = 5.3 Hz), 7.48 (m, 1H), 7.58-7.70 (m, 4H), 7.75-7.85 (m, 5H), 8.42 (t, 1H, J = 5.5 Hz), 9.06 (s, 1H); m/z (APCI pos) 550.1 (100%) [M + H]. |
| E60 | 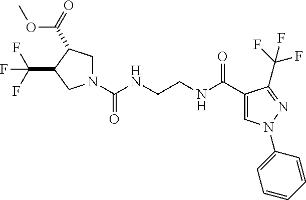 | trans-methyl 1-(2-(1- phenyl-3- (trifluoro- methyl)-1H- pyrazole-4- carboxamido) ethyl- carbamoyl)- 4- (trifluoro- methyl) pyrrolidine-3- carboxylate | White solid (9%); ¹H NMR (400 MHz, DMSO-d₆) δ 3.14-3.20 (m, 2H), 3.26-3.52 (m, 6H), 3.60-3.70 (m, 2H), 3.68 (s, 3H), 6.57 (t, 1H, J = 5.5 Hz), 7.48 (m, 1H), 7.58-7.64 (m, 2H), 7.79-7.83 (m, 2H), 8.39 (t, 1H, J = 5.5 Hz), 9.04 (s, 1H); m/z (APCI pos) 522.1 (100%) [M + H]. |
| E61 | 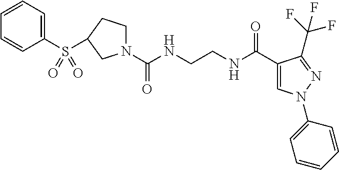 | 1-phenyl-N- (2-(3- (phenyl- sulfonyl) pyrrolidine-1- carboxamido) ethyl)-3- (trifluoro- methyl)-1H- pyrazole-4- carboxamide | White solid (51%); ¹H NMR (400 MHz, DMSO-d₆) δ 2.10-2.22 (m, 2H), 3.12-3.17 (m, 2H), 3.23-3.32 (m, 4H), 3.45 (dd, 1H, J = 8.1, 11.5 Hz), 3.60 (dd, 1H, J = 5.3, 11.5 Hz), 4.12-4.21 (m, 1H), 6.44 (t, 1H, J = 5.5 Hz), 7.48 (m, 1H), 7.56-7.71 (m, 4H), 7.75-7.84 (m, 3H), 7.88-7.92 (m, 2H), 8.39 (t, 1H, J = 5.5 Hz), 9.05 (s, 1H); m/z (APCI pos) 536.1 (100%) [M + H]. |
| E62 | 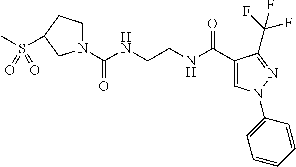 | N-(2-(3- (methyl- sulfonyl) pyrrolidine-1- carboxamido) ethyl)-1- phenyl-3- (trifluoro- methyl)-1H- pyrazole-4- carboxamide | White solid (37%); ¹H NMR (400 MHz, DMSO-d₆) δ 2.21-2.28 (m, 2H), 3.01 (s, 3H), 3.15- 3.21 (m, 2H), 3.26- 3.34 (m, 3H), 3.38- 3.46 (m, 1H), 3.57- 3.64 (m, 2H), 3.90- 3.97 (m, 1H), 6.46 (t, 1H, J = 5.5 Hz), 7.48 (m, 1H), 7.58-7.64 (m, 2H), 7.78-7.84 (m, 2H), 8.42 (t, 1H, J = 5.5 Hz), 9.05 (s, 1H); m/z (APCI pos) 474.1 (100%) [M + H]. |
| E63 | 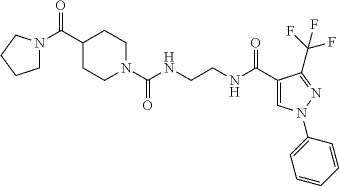 | N-(2-(1- phenyl-3- (trifluoro- methyl)-1H- pyrazole-4- carboxamido) ethyl)-4- (pyrrolidine- 1- carbonyl) piperidine-1- carboxamide | White solid (33%); ¹H NMR (400 MHz, DMSO-d₆ δ 1.34-1.45 (m, 2H), 1.55-1.62 (m, 2H), 1.71-1.79 (m, 2H), 1.83-1.90 (m, 2H), 2.50-2.74 (m, 3H), 3.15-3.31 (m, 6H), 3.47 (t, 2H, J = 6.7 Hz), 3.92-3.99 (m, 2H), 6.61 (t, 1H, J = 5.5 Hz), 7.48 (m, 1H), 7.58-7.64 (m, 2H), 7.78-7.84 (m, 2H), 8.41 (t, 1H, J = 5.5 Hz), 9.05 (s, 1H); m/z (APCI pos) 507.2 (100%) [M + H]. |
| E64 | 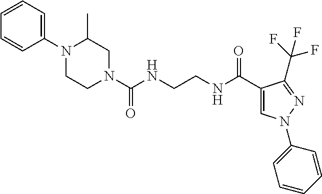 | 3-methyl-4- phenyl-N-(2- (1-phenyl-3- (trifluoro- methyl)-1H- pyrazole-4- carboxamido) ethyl) piperazine-1- carboxamide | White solid (69%); ¹H NMR (400 MHz, DMSO-d₆) δ 0.87 (d, 3H, J = 6.4 Hz), 2.87-3.06 (m, 2H), 3.16-3.25 (m, 4H), 3.28-3.35 (m, 2H), 3.68-3.74 (m, 1H), 3.86-3.94 (m, 2H), 6.70-6.78 (m, 2H), 6.87-6.92 (m, 2H), 7.18-7.23 (m, 2H), 7.48 (m, 1H), 7.58-7.64 (m, 2H), 7.78-7.84 (m, 2H), 8.40 (t, 1H, J = 5.5 Hz), 9.06 (s, 1H); m/z (APCI pos) 501.2 (100%) [M + H]. |
| E67 | 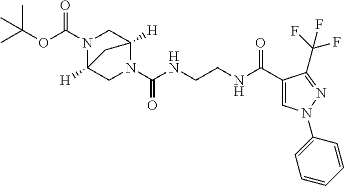 | (1S,4S)-(−)- tert-butyl 5- (2-(1-phenyl- 3- (trifluoro- methyl)-1H- pyrazole-4- carboxamido) ethylcarbamoyl)- 2,5- diazabicyclo [2.2.1]heptane- 2- carboxylate | White solid (88%); ¹H NMR (400 MHz, DMSO-d₆) δ 1.34-1.42 (m, 9H), 1.70-1.81 (m, 2H), 3.12-3.30 (m, 8H), 4.28-4.36 (m, 1H), 4.44 (s, 1H), 6.45 (t, 1H, J = 5.1 Hz), 7.48 (m, 1H), 7.58-7.64 (m, 2H), 7.78-7.84 (m, 2H), 8.36-8.44 (m, 1H), 9.05 (s, 1H); m/z (APCI pos) 423.1 (80%) [M + H − Boc]. |
| E68 | 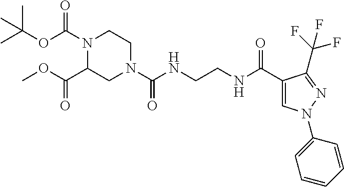 | 1-tert-butyl 2-methyl 4- (2-(1-phenyl- 3- (trifluoro- methyl)-1H- pyrazole-4- carboxamido) ethylcarbamoyl) piperazine- 1,2- dicarboxylate | White solid (76%); ¹H NMR (400 MHz, DMSO-d₆) δ 1.31-1.42 (m, 9H), 2.76-2.92 (m, 1H), 3.02-3.18 (m, 4H), 3.22-3.30 (m, 2H), 3.60 (s, 3H), 3.62- 3.70 (m, 1H), 3.76- 3.82 (m, 1H), 4.24- 4.38 (m, 1H), 4.46- 4.58 (m, 1H), 6.74 (m, 1H), 7.48 (m, 1H), 7.58-7.64 (m, 2H), 7.78-7.84 (m, 2H), 8.38 (t, 1H, J = 5.5 Hz), 9.06 (s, 1H); m/z (APCI pos) 569.1 (20%) [M + H], 469.1 (60%) [M + H − Boc]. |
| E69 | 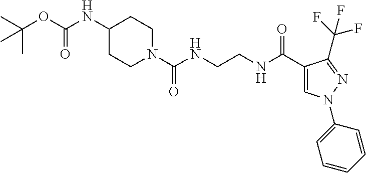 | tert-butyl 1- (2-(1-phenyl- 3- (trifluoro- methyl)-1H- pyrazole-4- carboxamido) ethylcarbamoyl) piperidin-4- ylcarbamate | White solid (84%); ¹H NMR (400 MHz, DMSO-d₆) δ 1.15-1.25 (m, 2H), 1.37 (s, 9H), 1.62- 1.69 (m, 2H), 2.67- 2.76 (m, 2H), 3.13- 3.19 (m, 2H), 3.24- 3.30 (m, 2H), 3.38 (br, 1H), 3.82-3.89 (m, 2H), 6.63 (t, 1H, J = 5.3 Hz), 6.79 (d, 1H, J = 7.8 Hz), 7.48 (m, 1H), 7.58-7.64 (m, 2H), 7.78-7.84 (m, 2H), 8.40 (t, 1H, J = 5.5 Hz), 9.06 (s, 1H); m/z (APCI pos) 425.1 (100%) [M + H − Boc]. |
| E80 | 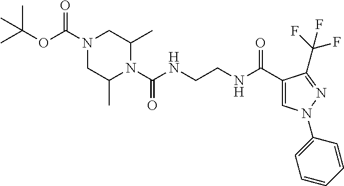 | tert-butyl 3,5-dimethyl- 4-(2-(1- phenyl-3- (trifluoro- methyl)-1H- pyrazole-4- carboxamido) ethylcarbamoyl) piperazine- 1-carboxylate | White solid (69%); ¹H NMR (400 MHz, DMSO-d₆) δ 0.99 (s, 3H), 1.01 (s, 3H), 1.35 (s, 9H), 2.75-2.95 (m, 2H), 3.12-3.28 (m, 4H), 3.70 (br, 2H), 3.90- 4.00 (m, 2H), 6.50 (t, 1H, J = 5.2 Hz), 7.48 (m, 1H), 7.58-7.64 (m, 2H), 7.78-7.84 (m, 2H), 8.32 (t, 1H, J = 5.5 Hz), 9.00 (s, 1H); m/z (APCI pos) 539.0 (60%) [M + H]. |
| E84 | 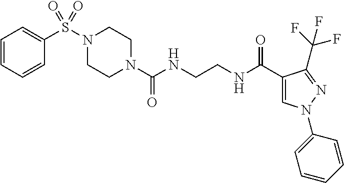 | N-(2-(1- phenyl-3- (trifluoro- methyl)-1H- pyrazole-4- carboxamido) ethyl)-4- (phenyl- sulfonyl) piperazine-1- carboxamide | White solid (78%); ¹H NMR (400 MHz, DMSO-d₆ δ 2.80-2.85 (m, 4H), 3.07-3.25 (m, 4H), 3.35-3.40 (m, 4H), 6.71 (t, 1H, J = 5.3 Hz), 7.45-7.50 (m, 1H), 7.58-7.82 (m, 9H), 8.34 (t, 1H, J = 5.5 Hz), 9.02 (s, 1H); m/z (APCI pos) 551.0 (100%) [M + H]. |
| E85 | 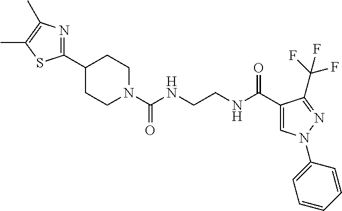 | 4-(4,5- dimethyl- thiazol-2-yl)-N- (2-(1-phenyl- 3- (trifluoro- methyl)-1H- pyrazole-4- carboxamido) ethyl) piperidine-1- carboxamide | White solid (57%); ¹H NMR (400 MHz, DMSO-d₆) δ 1.40-1.50 (m, 2H), 1.86-1.94 (m, 2H), 2.18 (s, 3H), 2.27 (s, 3H), 2.75-2.85 (m, 2H), 2.97-3.06 (m, 1H), 3.15-3.30 (m, 4H), 3.95-4.03 (m, 2H), 6.67 (t, 1H, J = 5.2 Hz), 7.48 (m, 1H), 7.58-7.64 (m, 2H), 7.78-7.84 (m, 2H), 8.39 (t, 1H; J = 5.5 Hz), 9.05 (s, 1H); m/z (APCI pos) 521.0 (100%) [M + H]. |
| E87 | 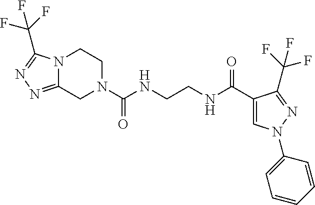 | N-(2-(1- phenyl-3- (trifluoro- methyl)-1H- pyrazole-4- carboxamido) ethyl)-3- (trifluoro- methyl)-5,6- dihydro- [1,2,4] triazolo[4,3- a]pyrazine- 7(8H)- carboxamide | White solid (71%); ¹H NMR (400 MHz, DMSO-d₆) δ 3.19-3.25 (m, 2H), 3.81-3.87 (m, 2H), 4.01-4.07 (m, 2H), 4.11-4.17 (m, 2H), 4.80 (s, 2H), 7.09 (t, 1H, J = 5.3 Hz), 7.48 (m, 1H), 7.58-7.64 (m, 2H), 7.78-7.84 (m, 2H), 8.38 (t, 1H, J = 5.5 Hz), 9.02 (s, 1H); m/z (APCI pos) 517.0 (100%) [M + H]. |
| E93 | 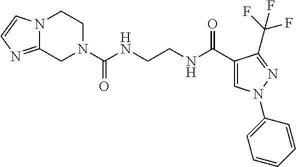 | N-(2-(1- phenyl-3- (trifluoro- methyl)-1H- pyrazole-4- carboxamido) ethyl)-5,6- dihydroimidazo [1,2- a]pyrazine- 7(8H)- carboxamide | White solid (48%); ¹H NMR (400 MHz, DMSO-d₆) δ 3.17-3.34 (m, 4H), 3.69 (m, 2H), 4.01 (m, 2H), 4.55 (s, 2H), 6.70 (s, 1H), 6.89 (t, 1H, J = 5.3 Hz), 7.48 (m, 1H), 7.55 (s, 1H), 7.58-7.64 (m, 2H), 7.78-7.84 (m, 2H), 8.41 (t, 1H, J = 5.5 Hz), 9.04 (s, 1H); m/z (APCI pos) 448.2 (100%) [M + H]. |
| E94 | 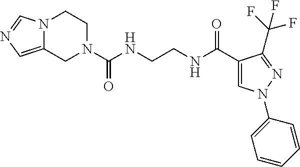 | N-(2-(1- phenyl-3- (trifluoro- methyl)-1H- pyrazole-4- carboxamido) ethyl)-5,6- dihydroimidazo [1,5- a]pyrazine- 7(8H)- carboxamide | White solid (64%); ¹H NMR (400 MHz, DMSO-d₆) δ 3.17-3.32 (m, 4H), 3.75 (m, 2H), 3.95 (m, 2H), 4.53 (s, 2H), 6.85 (s, 1H), 6.95 (t, 1H, J = 5.1 Hz), 7.06 (s, 1H), 7.48 (m, 1H), 7.58-7.64 (m, 2H), 7.78-7.84 (m, 2H), 8.40 (t, 1H, J = 5.5 Hz), 9.04 (s, 1H); m/z (APCI pos) 448.1 (100%) [M + H]. |
| E95 | 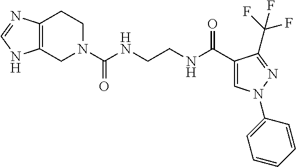 | N-(2-(1- phenyl-3- (trifluoro- methyl)-1H- pyrazole-4- carboxamido) ethyl)-6,7- dihydro-3H- imidazo[4,5- c]pyridine- 5(4H)- carboxamide | White solid (67%); ¹H NMR (400 MHz, DMSO-d₆) δ 2.54 (m, 2H), 3.16- 3.32 (m, 4H), 3.60 (t, 2H, J = 5.5 Hz), 4.30 (s, 2H), 6.75 (t, 1H, J = 5.3 Hz), 7.45-7.50 (m, 1H), 7.46 (s, 1H), 7.58-7.64 (m, 2H), 7.78-7.84 (m, 2H), 8.40 (t, 1H, J = 5.5 Hz), 9.05 (s, 1H), 11.80 (br, 1H); m/z (APCI pos) 448.0 (100%) [M + H]. |
| E96 | 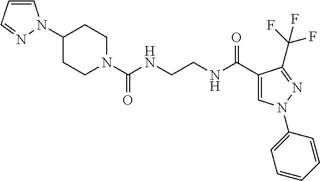 | N-(2-(1- phenyl-3- (trifluoro- methyl)-1H- pyrazole-4- carboxamido) ethyl)-4-(1H- pyrazol-1- yl)piperidine- 1- carboxamide | White solid (70%); ¹H NMR (400 MHz, DMSO-d₆) δ 1.68-1.80 (m, 2H), 1.90-1.98 (m, 2H), 2.78-2.88 (m, 2H), 3.16-3.33 (m, 4H), 4.00-4.09 (m, 2H), 4.28-4.36 (m, 1H), 6.21 (t, 1H, J = 2.0 Hz), 6.74 (t, 1H, J = 5.3 Hz), 7.42 (m, 1H), 7.48 (m, 1H), 7.58- 7.64 (m, 2H), 7.74 (m, 1H), 7.78-7.84 (m, 2H), 8.41 (t, 1H, J = 5.5 Hz), 9.06 (s, 1H); m/z (APCI pos) 476.0 (50%) [M + H]. |
| E101 | 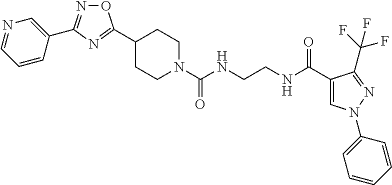 | N-(2-(1- phenyl-3- (trifluoro- methyl)-1H- pyrazole-4- carboxamido) ethyl)-4-(3- (pyridin-3- yl)-1,2,4- oxadiazol-5- yl)piperidine- 1- carboxamide | White solid (90%); ¹H NMR (400 MHz, DMSO-d₆) δ 1.62-1.74 (m, 2H), 2.02-2.10 (m, 2H), 2.90-2.99 (m, 2H), 3.18-3.40 (m, 5H), 3.94-4.00 (m, 2H), 6.72 (t, 1H, J = 5.3 Hz), 7.44-7.50 (m, 1H), 7.57-7.64 (m, 3H), 7.78-7.82 (m, 2H), 8.32-8.36 (m, 1H), 8.40 (t, 1H, J = 5.5 Hz), 8.76-8.79 (m, 1H), 9.05 (s, 1H), 9.15 (m, 1H); m/z (APCI pos) 555.2 (100%) [M + H]. |
| E104 | 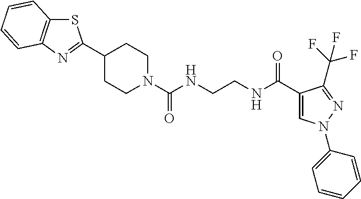 | 4- (benzo[d] thiazol-2-yl)-N- (2-(1-phenyl- 3- (trifluoro- methyl)-1H- pyrazole-4- carboxamido) ethyl) piperidine-1- carboxamide | White solid (85%); ¹H NMR (400 MHz, DMSO-d₆) δ 1.59-1.71 (m, 2H), 2.03-2.10 (m, 2H), 2.85-2.93 (m, 2H), 3.17-3.34 (m 5H), 4.02-4.09 (m, 2H), 6.71 (t, 1H, J = 5.3 Hz), 7.38-7.51 (m, 3H), 7.57-7.63 (m, 2H), 7.77-7.81 (m, 2H), 7.92-7.95 (m, 1H), 8.03-8.07 (m, 1H), 8.41 (t, 1H, J = 5.5 Hz), 9.05 (s, 1H); m/z (APCI pos) 543.2 (100%) [M + H]. |
| E106 | 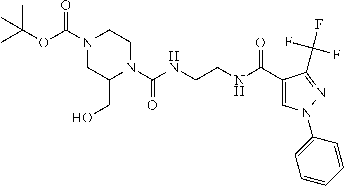 | tert-butyl 3- (hydroxy- methyl)-4-(2-(1- phenyl-3- (trifluoro- methyl)-1H- pyrazole-4- carboxamido) ethylcarbamoyl) piperazine- 1-carboxylate | White solid (55%); ¹H NMR (400 MHz, DMSO-d₆) δ 1.40 (s, 9H), 2.70- 2.95 (m, 3H), 3.15- 3.42 (m, 6H), 3.68- 3.78 (m, 2H), 3.86- 3.98 (m 2H), 4.75 (t, 1H, J = 4.9 Hz), 6.58 (t, 1H, J = 5.3 Hz), 7.46-7.50 (m, 1H), 7.58-7.64 (m, 2H), 7.79-7.83 (m, 2H), 8.38 (t, 1H, J = 5.5 Hz), 9.04 (s, 1H); m/z (APCI pos) 441.2 (24%) [M + H − Boc]. |
| E109 | 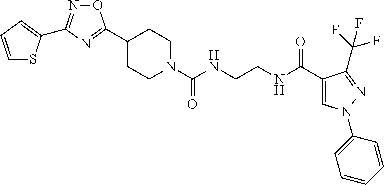 | N-(2-(1- phenyl-3- (trifluoro- methyl)-1H- pyrazole-4- carboxamido) ethyl)-4-(3- (thiophen-2- yl)-1,2,4- oxadiazol-5- yl)piperidine- 1- carboxamide | White solid (95%); ¹H NMR (400 MHz, DMSO-d₆) δ 1.58-1.70 (m, 2H), 1.98-2.07 (m, 2H), 2.87-2.96 (m, 2H), 3.18-3.40 (m, 5H), 3.94-4.00 (m, 2H), 6.72 (t, 1H, J = 5.3 Hz), 7.24-7.28 (m, 1H), 7.44-7.50 (m, 1H), 7.58-7.64 (m, 2H), 7.77-7.84 (m, 3H), 7.86-7.88 (m, 1H), 8.40 (t, 1H, J = 5.5 Hz), 9.05 (s, 1H); m/z (APCI pos) 560.2 (100%) [M + H]. |
| E145 | 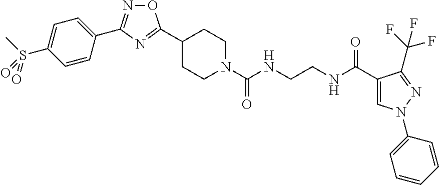 | 4-(3-(4- (methyl- sulfonyl) phenyl)- 1,2,4- oxadiazol-5- yl)-N-(2-(1- phenyl-3- (trifluoro- methyl)-1H- pyrazole-4- carboxamido) ethyl) piperidine-1- carboxamide | Colorless crystals (82%); ¹H NMR (400 MHz, CDCl₃) δ 1.86-1.99 (2H, m), 2.13-2.22 (2H, m), 3.02-3.13 (2H, m), 3.10 (3H, s), 3.23 (1H, m), 3.48- 3.55 (2H, m), 3.56- 3.64 (2H, m), 4.01 (2H, dt, J = 3.6, 10.0 Hz), 5.39 (1H, t, J = 4.8 Hz), 7.04 (1H, br), 7.42 (1H, t, J = 7.2 Hz), 7.47-7.54 (2H, m), 7.68-7.74 (2H, m), 8.06 (2H, d, J = 8.4 Hz), 8.27 (2H, d, J = 8.4 Hz), 8.42 (1H, d, J = 0.8 Hz); m/z (APCI pos) 632.0 (100%) (M + H). |
| E146 | 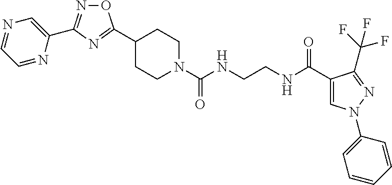 | N-(2-(1- phenyl-3- (trifluoro- methyl)-1H- pyrazole-4- carboxamido) ethyl)-4-(3- (pyrazin-2- yl)-1,2,4- oxadiazol-5- yl)piperidine- 1- carboxamide | ¹H NMR (400 MHz, CDCl₃) δ 1.91-2.06 (2H, m), 2.14-2.25 (2H, m), 3.02-3.13 (2H, m), 3.27 (1H, m), 3.46- 3.55 (2H, m), 3.55- 3.64 (2H, m), 3.96- 4.07 (2H, m), 5.43 (1H, t, J= 4.8 Hz), 7.13 (1H, br) 7.41 (1H, t, J = 7.6 Hz), 7.51 (2H, t, J = 7.6 Hz), 7.71 (2H, d, J = 7.6 Hz), 8.44 (1H, d, J = 0.8 Hz), 8.71-8.78 (2H, m), 9.35 (1H, d, J = 1.6 Hz); m/z (APCI pos) 556.1 (100%) (M + H). |
| E147 | 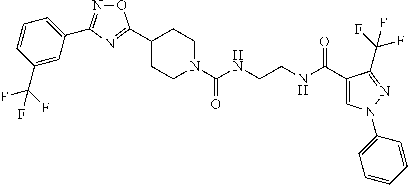 | N-(2-(1- phenyl-3- (trifluoro- methyl)-1H- pyrazole-4- carboxamido) ethyl)-4-(3- (3- (trifluoro- methyl) phenyl)- 1,2,4- oxadiazol-5- yl)piperidine- 1- carboxamide | ¹H NMR (400 MHz, CDCl₃) δ 1.87-2.00 (2H, m), 2.12-2.22 (2H, m), 3.02-3.14 (2H, m), 3.22 (1H, m), 3.46- 3.55 (2H, m), 3.55- 3.64 (2H, m), 3.96- 4.06 (2H, m), 5.39 (1H, t, J = 4.8 Hz), 7.11 (1H, br), 7.41 (1H, t, J = 7.6 Hz), 7.50 (2H, t, J = 7.6 Hz), 7.61 (1H, t, J = 7.6 Hz), 7.71 (2H, d, J = 7.6 Hz), 7.76 (1H, d, J = 7.6 Hz), 8.25 (1H, d, J = 7.6 Hz), 8.34 (1H, s), 8.43 (1H, s); m/z (APCI pos) 622.0 (100%) (M + H). |

### Example E12

### 1-Butyryl-N-(2-(1-phenyl-3-(trifluoromethyl)-1H-pyrazole-4-carboxamido)ethyl)piperidine-4-carboxamide

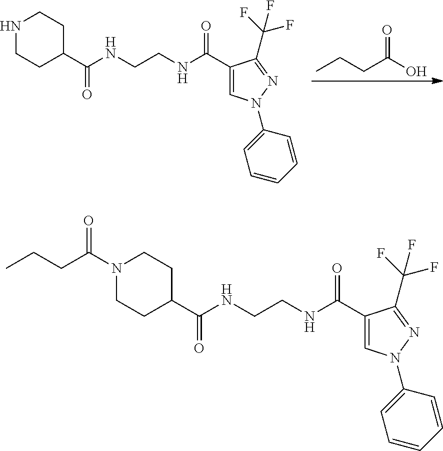

To N-(2-(1-phenyl-3-(trifluoromethyl)-1H-pyrazole-4-carboxamido)ethyl)piperidine-3-carboxamide (200 mg, 0.49 mmol) in DCM (50 mL) were added successively butyric acid (43 mg, 0.49 mmol), EDAC.HCl (122 mg, 0.63 mmol), HOBt.H₂O (93 mg, 0.68 mmol) and triethylamine (99 mg, 0.98 mmol). The mixture was stirred at room temperature for 12 hours then DCM (50 mL) was added. The organic layer was successively washed with 2N HCl (50 mL), 10% aqueous K₂CO₃ (50 mL) and brine (50 mL) then dried over MgSO₄. After concentration and purification by MPLC (DCM/MeOH 98/2), 1-butyryl-N-(2-(1-phenyl-3-(trifluoromethyl)-1H-pyrazole-4-carboxamido)ethyl)piperidine-4-carboxamide was isolated as a white solid (1.2 g, 67%): ¹H NMR (400 MHz, DMSO-d₆) δ 0.86 (t, 3H, J=7.4 Hz), 1.28-1.45 (m, 3H), 1.63-1.75 (m, 2H), 2.24 (dd, 2H, J=7.4, 1.9 Hz), 2.33 (m, 1H), 2.53 (m, 1H), 2.98 (m, 1H), 3.16-3.33 (m, 5H), 3.84 (d, 1H, J=12.9 Hz), 4.34 (d, 1H, J=12.9 Hz), 7.48 (m, 1H), 7.61 (m, 2H), 7.81 (m, 2H), 7.92 (t, 1H, J=5.5 Hz), 8.35 (t, 1H, J=5.5 Hz), 9.03 (s, 1H); m/z (APCI pos) 480.2 (100%) [H+H].

Compounds of the following structures were prepared from the corresponding acids and amines, using a similar method to that described above.

| Ex # | Structure | Name | Physical Data |
|---|---|---|---|
| E10 | 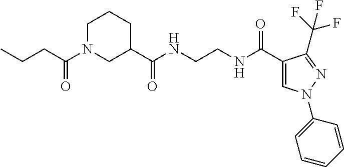 | 1-butyryl-N- (2-(1- phenyl-3- (trifluoro- methyl)- 1H- pyrazole-4- carboxamido) ethyl) piperidine- 3- carboxamide | White solid (52%); ¹H NMR (400 MHz, DMSO-d₆) δ 0.85 (t, 3H, J = 7.4 Hz), 1.22-1.32 (m, 1H), 1.40-1.85 (m, 5H), 2.10-2.30 (m, 3H), 2.50-2.70 (m, 1H), 2.90-3.10 (m, 1H), 3.15-3.34 (m, 4H), 3.79 (d, 1H, J = 13.7 Hz), 4.22 (d, 0.5H, J = 12.1 Hz), 4.37 (d, 0.5H, J = 12.1 Hz), 7.48 (m, 1H), 7.61 (m, 2H), 7.82 (m, 2H), 8.02 (t, 1H, J = 5.0 Hz), 8.38 (m, 1H), 9.04 (d, 1H, J = 3.5 Hz); m/z (APCI pos) 480.2 (100%) [M + H]. |
| E27 | 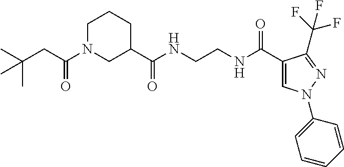 | 1-(3,3- dimethyl- butanoyl)- N-(2- (1-phenyl-3- (trifluoro- methyl)- 1H- pyrazole-4- carboxamido) ethyl) piperidine-3- carboxamide | White solid (33%); ¹H NMR (400 MHz, DMSO-d₆) δ 0.92 (s, 3H), 0.97 (s, 6H), 1.20-1.40 (m, 1H), 1.52-1.72 (m, 2H), 1.78-1.90 (m, 1H), 2.10-2.30 (m, 3H), 2.64 (dd, 1H, J = 10.9, 1.5 Hz), 2.90- 3.34 (m, 5H), 3.86 (d, 1H, J = 12.9 Hz), 4.25-4.45 (m, 1H), 7.48 (m, 1H), 7.60 (m, 2H), 7.81 (m, 2H), 8.01 (t, 1H, J = 5.5 Hz), 8.36 (m, 1H), 9.04 (d, 1H); m/z (APCI pos) 508.2 (100%) [M + H]. |
| E28 | 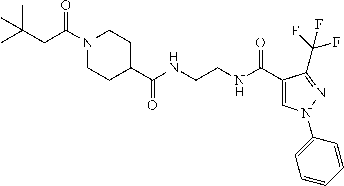 | 1-(3,3- dimethyl- butanoyl)- N-(2- (1-phenyl-3- (trifluoro- methyl)-1H- pyrazole-4- carboxamido) ethyl) piperidine-4- carboxamide | White solid (50%) ; ¹H NMR (400 MHz, DMSO-d₆) δ 0.96 (s, 9H), 1.27- 1.50 (m, 2H), 1.65- 1.74 (m, 2H), 2.20 (s, 2H), 2.34 (m, 1H), 1.98 (t, 1H, J = 12.1 Hz), 3.18-3.34 (m, 5H), 3.94 (d, 1H, J = 13.7 Hz), 4.40 (d, 1H, J = 12.9 Hz), 7.48 (m, 1H), 7.60 (m, 2H), 7.81 (m, 2H), 7.93 (t, 1H, J = 5.5 Hz), 8.36 (t, 1H, J = 5.5 Hz), 9.04 (s, 1H); m/z (APCI pos) 508.2 (100%) [M + H]. |

### Example E39

### N-(2-(1-Phenyl-3-(trifluoromethyl)-1H-pyrazole-4-carboxamido)ethyl)-4-(trifluoromethyl)piperidine-1-carboxamide

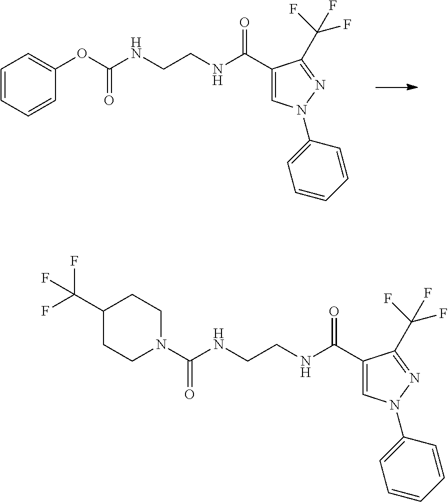

To phenyl 2-(1-phenyl-3-(trifluoromethyl)-1H-pyrazole-4-carboxamido)ethylcarbamate (209 mg, 0.5 mmol) were added 4-(trifluoromethyl)piperidine (77 mg, 0.5 mmol), cesium carbonate (163, 0.5 mmol) and EtOH (0.5 mL). The mixture was heated to 160° C. for 30 minutes then cooled down and purified by MPLC (DCM/MeOH 97/3) to yield N-(2-(1-phenyl-3-(trifluoromethyl)-1H-pyrazole-4-carboxamido)ethyl)-4-(trifluoromethyl)piperidine-1-carboxamide as a white solid (78 mg, 33%). Crystallized from AcOEt/hexanes: ¹H NMR (400 MHz, DMSO-d₆) δ 1.15-1.32 (m, 2H), 1.70-1.80 (m, 2H), 2.64-2.74 (m, 2H), 3.15-3.22 (m, 2H), 3.26-3.33 (m, 4H), 4.00-4.08 (m, 1H), 6.70 (t, 1H, J=5.5 Hz), 7.45-7.50 (m, 1H), 7.58-7.64 (m, 2H), 7.78-7.84 (m, 2H), 8.38 (t, 1H, J=5.5 Hz), 9.05 (s, 1H); m/z (APCI pos) 478.1 (100%) [M+H].

The compound of the following structure was prepared from the corresponding amine, using a similar method to that described above:

| Ex # | Structure | Name | Physical Data |
|---|---|---|---|
| E40 | 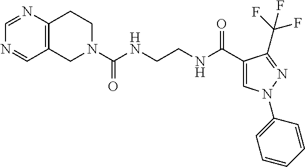 | N-(2-(1- phenyl-3- (trifluoro- methyl)-1H- pyrazole-4- carboxamido) ethyl)-7,8- dihydro pyrido[4,3- d]pyrimidine- 6(5H)- carboxamide | Yellow solid (36%); ¹H NMR (400 MHz, DMSO-d₆) δ 2.84 (t, 2H, J = 5.9 Hz), 3.20-3.34 (m, 4H), 3.67 (t, 2H, J = 5.9 Hz), 4.56 (s, 2H), 6.94 (t, 1H, J = 5.5 Hz), 7.45-7.50 (m, 1H), 7.58-7.64 (m, 2H), 7.78-7.84 (m, 2H), 8.43 (t, 1H, J = 5.5 Hz), 8.55 (s, 1H), 8.92 (s, 1H), 9.05 (s, 1H); m/z (APCI pos) 460.0 (100%) [M + H]. |

### Example E41

### tert-Butyl 3-methyl-4-(2-(1-phenyl-3-(trifluoromethyl)-1H-pyrazole-4-carboxamido)ethylcarbamoyl)piperidine-1-carboxylate

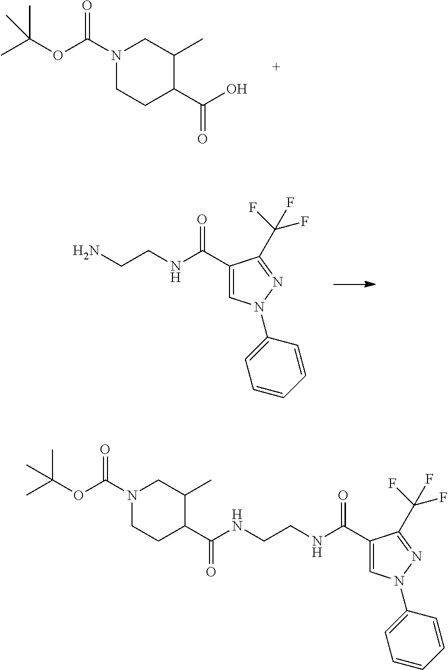

To N-(2-aminoethyl)-1-phenyl-3-(trifluoromethyl)-1H-pyrazole-4-carboxamide (613 mg, 2.06 mmol) in DCM (100 mL) were successively added 1-(tert-butoxycarbonyl)-3-methylpiperidine-4-carboxylic acid (500 mg, 2.06 mmol), EDAC.HCl (512 mg, 2.67 mmol), HOBt.H₂O (389 mg, 2.88 mmol) and triethylamine (416 mg, 4.11 mmol). The mixture was stirred at room temperature for 18 hours then DCM was added (100 mL). The organic layer was successively washed with 1N HCl (100 mL), 10% potassium carbonate aqueous solution (100 mL) and brine (2×200 mL). The organic layer was dried over MgSO₄ then concentrated to yield a white solid. The crude solid was purified by MPLC (DCM/MeOH 99/1) to yield tert-butyl 3-methyl-4-(2-(1-phenyl-3-(trifluoromethyl)-1H-pyrazole-4-carboxamido) ethylcarbamoyl)piperidine-1-carboxylate as a white solid (730 mg, 68%). Crystallized from AcOEt/hexanes: ¹H NMR (400 MHz, DMSO-d₆) δ 0.73 (d, 3H, J=6.9 Hz), 1.37 (s, 9H), 1.38-1.44 (m, 1H), 1.60-1.70 (m, 1H), 1.93-2.03 (m, 1H), 2.37-2.43 (m, 1H), 2.72-3.10 (m, 2H), 3.18-3.30 (m, 4H), 3.64 (dd, 1H, J=3.9, 12.9 Hz), 3.82 (br, 1H), 7.48 (m, 1H), 7.60 (m, 2H), 7.77-7.88 (m, 3H), 8.35 (t, 1H, J=5.5 Hz), 9.04 (s, 1H); m/z (APCI pos) 424.1 (100%) [M+H-Boc].

Compounds of the following structures were prepared from the corresponding acids, using a similar method to that described above.

| Ex # | Structure | Name | Physical Data |
|---|---|---|---|
| E34 | 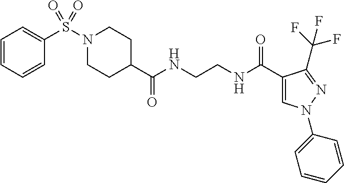 | N-(2-(1- phenyl-3- (trifluoro- methyl)-1H- pyrazole-4- carboxamido) ethyl)-1- (phenyl- sulfonyl) piperidine- 4- carboxamide | White solid (49%); ¹H NMR (400 MHz, DMSO-d₆) δ 1.50-1.60 (m, 2H), 1.72-1.80 (m, 2H), 2.07 (m, 1H), 2.24- 2.32 (m, 2H), 3.14- 3.28 (m, 4H), 3.56- 3.62 (m, 2H), 7.46- 7.50 (m, 1H), 7.58- 7.66 (m, 4H), 7.68- 7.75 (m, 3H), 7.78- 7.82 (m, 2H), 7.85 (t, 1H, J = 5.8 Hz), 8.34 (t, 1H, J = 5.5 Hz), 9.02 (s, 1H); m/z (APCI pos) 550.1 (100%) [M + H]. |
| E45 | 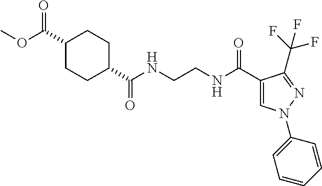 | cis-methyl 4- (2-(1-phenyl- 3- (trifluoro- methyl)-1H- pyrazole-4- carboxamido) ethyl- carbamoyl) cyclohexane- carboxylate | White solid (69%); ¹H NMR (400 MHz, DMSO-d₆) δ 1.45-1.57 (m, 6H), 1.88-1.96 (m, 2H), 2.17 (m, 1H), 2.55 (m, 1H), 3.16-3.30 (m, 4H), 3.58 (s, 3H), 7.46-7.50 (m, 1H), 7.58-7.63 (m, 2H), 7.77-7.83 (m, 3H), 8.34 (t, 1H, J = 5.5 Hz), 9.04 (s, 1H); m/z (APCI pos) 467.1 (100%) [M + H]. |
| E46 | 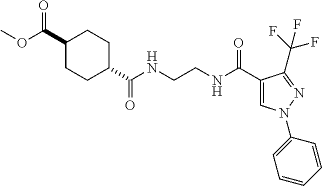 | trans-methyl 4-(2-(1- phenyl-3- (trifluoro- methyl)-1H- pyrazole-4- carboxamido) ethyl- carbamoyl) cyclohexane- carboxylate | White solid (92%); ¹H NMR (400 MHz, DMSO-d₆) δ 1.23-1.43 (m, 4H), 1.72-1.81 (m, 2H), 1.87-1.95 (m, 2H), 2.05 (m, 1H), 2.25 (m, 1H), 3.16-3.30 (m, 4H), 3.58 (s, 3H), 7.46-7.50 (m, 1H), 7.58-7.63 (m, 2H), 7.77-7.83 (m, 3H), 8.36 (t, 1H, J = 5.5 Hz), 9.04 (s, 1H); m/z (APCI pos) 467.1 (100%) [M + H]. |
| E65 | 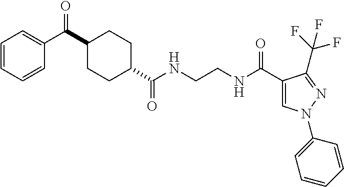 | N-(2-(trans- 4- benzoylcyclo- hexane- carboxamido) ethyl)-1- phenyl-3- (trifluoro- methyl)-1H- pyrazole-4- carboxamide | White solid (24%); ¹H NMR (400 MHz, DMSO-d₆) δ 1.28-1.40 (m, 2H), 1.50-1.63 (m, 2H), 1.78-1.89 (m, 4H), 2.06-2.15 (m, 1H), 3.17-3.30 (m, 4H), 3.34-3.44 (m, 1H), 7.45-7.55 (m, 3H), 7.58-7.65 (m, 3H), 7.79-7.84 (m, 2H), 7.87 (t, 1H, J = 5.5 Hz), 7.95-7.99 (m, 2H), 8.37 (t, 1H, J = 5.5 Hz), 9.04 (s, 1H); m/z (APCI pos) 513.1 (100%) [M + H]. |
| E66 | 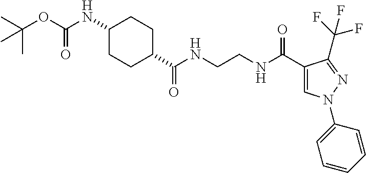 | tert-butyl cis-4-(2-(1- phenyl-3- (trifluoro- methyl)-1H- pyrazole-4- carboxamido) ethyl- carbamoyl) cyclohexyl- carbamate | White solid (65%); ¹H NMR (400 MHz, DMSO-d₆) δ 1.36 (s, 9H), 1.38- 1.47 (m, 4H), 1.56- 1.64 (m, 2H), 1.70- 1.80 (m, 2H), 2.10- 2.16 (m, 1H), 3.17- 3.30 (m, 4H), 3.43 (br, 1H), 6.67 (m, 1H), 7.48 (m, 1H), 7.58-7.64 (m, 2H), 7.76 (t, 1H, J = 5.5 Hz), 7.78-7.84 (m, 2H), 8.37 (t, 1H, J = 5.5 Hz), 9.03 (s, 1H); m/z (APCI pos) 424.2 (100%) [M + H − Boc]. |
| E75 | 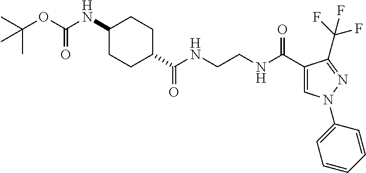 | tert-butyl trans-4-(2- (1-phenyl-3- (trifluoro- methyl)-1H- pyrazole-4- carboxamido) ethyl- carbamoyl) cyclohexyl- carbamate | White solid (88%); ¹H NMR (400 MHz, DMSO-d₆) δ 1.06-1.18 (m, 2H), 1.26-1.34 (m, 2H), 1.37 (s, 9H), 1.68- 1.87 (m, 4H), 1.92- 2.02 (m, 1H), 3.10- 3.30 (m, 5H), 6.68 (m, 1H), 7.48 (m, 1H), 7.58-7.64 (m, 2H), 7.78-7.84 (m, 3H), 8.35 (m, 1H), 9.03 (s, 1H); m/z (APCI pos) 424.0 (100%) [M + H − Boc]. |
| E77 | 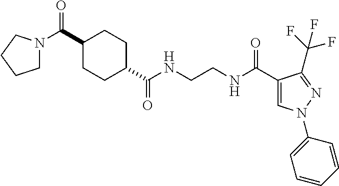 | 1-phenyl-N- (2-(trans-4- (pyrrolidine- 1- carbonyl) cyclohexane- carboxamido) ethyl)- 3- (trifluoro- methyl)-1H- pyrazole-4- carboxamide | White solid (62%); ¹H NMR (400 MHz, DMSO-d₆) δ 1.25-1.45 (m, 4H), 1.65-1.90 (m, 8H), 2.02-2.12 (m, 1H), 2.30-2.40 (m, 1H), 3.14-3.30 (m, 6H), 3.40-3.46 (m, 2H), 7.48 (m, 1H), 7.58- 7.64 (m, 2H), 7.78- 7.84 (m, 3H), 8.40 (t, 1H, J = 5.2 Hz), 9.02 (s, 1H); m/z (APCI pos) 506.1 (100%) [M + H]. |
| E78 | 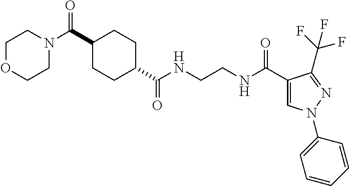 | N-(2-(trans- 4- (morpholine- 4- carbonyl) cyclohexane- carboxamido) ethyl)- 1-phenyl-3- (trifluoro- methyl)-1H- pyrazole-4- carboxamide | White solid (89%); ¹H NMR (400 MHz, DMSO-d₆) δ 1.26-1.50 (m, 4H), 1.42-1.78 (m, 4H), 2.02-2.12 (m, 1H), 2.52-2.58 (m, 1H), 3.15-3.30 (m, 4H), 3.36-3.58 (m, 8H), 7.48 (m, 1H), 7.58- 7.64 (m, 2H), 7.78- 7.84 (m, 3H), 8.40 (m, 1H), 9.02 (s, 1H); m/z (APCI pos) 522.1 (100%) [M + H]. |
| E79 | 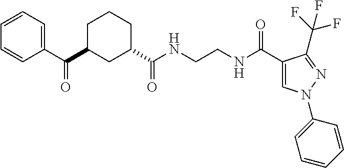 | N-(2-(trans- 3- benzoylcyclo- hexane- carboxamido) ethyl)-1- phenyl-3- (trifluoro- methyl)-1H- pyrazole-4- carboxamide | White solid (24%); ¹H NMR (400 MHz, DMSO-d₆) δ 1.22-1.34 (m, 2H), 1.40-1.52 (m, 2H), 1.75-1.90 (m, 4H), 2.26-2.36 (m, 1H), 3.15-3.30 (m, 4H), 3.38-3.48 (m, 1H), 7.44-7.54 (m, 3H), 7.56-7.66 (m, 3H), 7.77-7.82 (m, 2H), 7.86 (t, 1H, J = 5.5 Hz), 7.94-7.98 (m, 2H), 8.34 (t, 1H, J = 5.5 Hz), 9.00 (s, 1H); m/z (APCI pos) 513.1 (100%) [M + H]. |
| E88 | 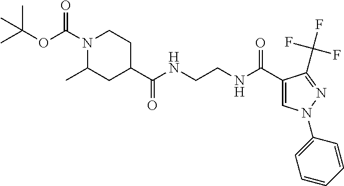 | tert-butyl 2- methyl-4-(2- (1-phenyl-3- (trifluoro- methyl)-1H- pyrazole-4- carboxamido) ethyl- carbamoyl) piperidine- 1-carboxylate | White solid (4%); ¹H NMR (400 MHz, DMSO-d₆) δ 1.11 (d, 3H, J = 6.64 Hz), 1.44 (s, 9H), 1.64 (m, 1H), 1.85 (m, 3H), 2.36 (m, 1H), 3.08 (m, 1H), 3.49 (m, 2H), 3.57 (m, 2H), 3.78 (m, 1H), 3.97 (m, 1H), 6.63 (m, 1H), 7.03 (m, 1H), 7.41 (m, 1H), 7.51 (m, 2H), 7.70 (m, 2H), 8.44 (s, 1H); m/z (APCI pos) 424.1 (100%) [M − BOC + H]. |
| E105 | 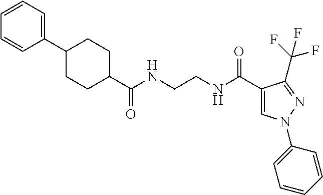 | 1-phenyl-N- (2-(4- phenylcyclo- hexane- carboxamido) ethyl)-3- (trifluoro- methyl)-1H- pyrazole-4- carboxamide | White solid (96%); ¹H NMR (400 MHz, DMSO-d₆) δ 1.35-1.57 (m, 4H), 1.80-1.87 (m, 4H), 2.10-2.20 (m, 1H), 2.43-2.51 (m 1H), 3.18-3.32 (m, 4H), 7.14-7.30 (m, 5H), 7.46-7.50 (m, 1H), 7.58-7.64 (m, 2H), 7.79-7.83 (m, 2H), 7.87 (t, 1H, J = 5.5 Hz), 8.37 (t, 1H, J = 5.5 Hz), 9.05 (s, 1H); m/z (APCI pos) 485.2 (100%) [M + H]. |
| E117 | 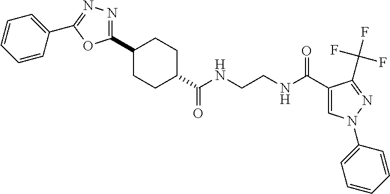 | 1-phenyl-N- (2-(trans-4- (5-phenyl- 1,3,4- oxadiazol-2- yl) cyclohexane- carboxamido) ethyl)-3- (trifluoro- methyl)-1H- pyrazole-4- carboxamide | White solid (38%); ¹H NMR (400 MHz, CDCl₃) δ 1.58-1.78 (4H, m), 2.02-2.12 (2H, m), 2.23 (1H, m), 2.26- 2.36 (2H, m), 2.97 (1H, m), 3.48-3.56 (2H, m), 3.58-3.66 (2H, m), 6.42 (1H, t, J = 4.8 Hz), 6.86 (1H, br), 7.42 (1H, m), 7.46-7.56 (5H, m), 7.72 (2H, d, J = 8.8 Hz), 8.02 (2H, dd, J = 1.6, 8.0 Hz), 8.43 (1H, s); m/z (APCI pos) 553.1 (100%) [M + H]. |
| E156 | 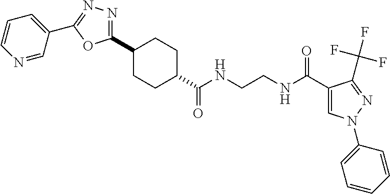 | 1-phenyl-N- (2-(trans-4- (5-(pyridin- 3-yl)-1,3,4- oxadiazol-2- yl) cyclohexane- carboxamido) ethyl)-3- (trifluoro- methyl)-1H- pyrazole-4- carboxamide | ¹H NMR (400 MHz, CDCl₃) δ 1.58-1.80 (4H, m), 2.04-2.12 (2H, m), 2.22 (1H, m), 2.28- 2.36 (2H, m), 3.00 (1H, m), 3.50-3.56 (2H, m), 3.58-3.66 (2H, m), 6.28 (1H, t, J = 5.2 Hz), 6.72 (1H, br), 7.40-7.48 (2H, m), 7.48-7.56 (2H, m), 7.72 (2H, d, J = 7.6 Hz), 8.33 (1H, dt, J = 2.0, 8.0 Hz), 8.42 (1H, s), 8.77 (1H, dd, J = 1.6, 4.8 Hz), 9.24 (1H, d, J = 2.0 Hz); m/z (APCI pos) 554.2 (100%) [M + H]. |
| E159 | 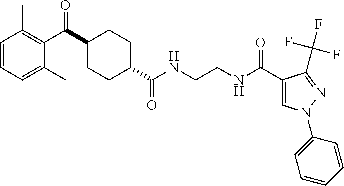 | N-(2-(trans- 4-(2,6- dimethyl- benzoyl) cyclohexane- carboxamido) ethyl)-1- phenyl-3- (trifluoro- methyl)-1H- pyrazole-4- carboxamide | Colorless crystals (73%); ¹H NMR (400 MHz, CDCl₃) δ 1.41-1.56 (4H, m), 1.92-2.17 (5H, m), 2.21 (6H, s), 2.70 (1H, m), 3.45-3.54 (2H, m), 3.55-3.64 (2H, m), 6.20 (1H, t, J = 4.8 Hz), 6.76 (1H, br), 7.01 (2H, d, J = 7.6 Hz), 7.16 (1H, t, J = 7.6 Hz), 7.42 (1H, t, J = 7.6 Hz), 7.51 (2H, t, J = 7.6 Hz), 7.70 (2H, d, J = 7.6 Hz), 8.40 (1H, s); m/z (APCI pos) 541.1 (100%) [M + H]. |
| E160 | 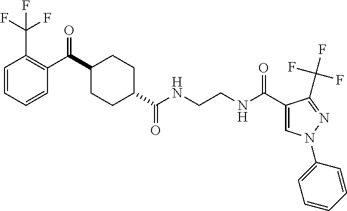 | 1-phenyl-3- (trifluoro- methyl)-N-(2- (trans-4-(2- (trifluoro- methyl) benzoyl) cyclohexane- carboxamido) ethyl)-1H- pyrazole-4- carboxamide | ¹H NMR (400 MHz, CDCl₃) δ 1.40-1.59 (4H, m), 1.94-2.06 (4H, m), 2.11 (1H, m), 2.90 (1H, m), 3.46-3.54 (2H, m), 3.55-3.63 (2H, m), 6.20 (1H, t, J = 5.2 Hz), 6.75 (1H, br), 7.39 (1H, m), 7.43 (1H, m), 7.47- 7.63 (4H, m), 7.67- 7.74 (3H, m), 8.40 (1H, d, J = 0.8 Hz); m/z (APCI pos) 581.1 (100%) [M + H]. |
| E161 | 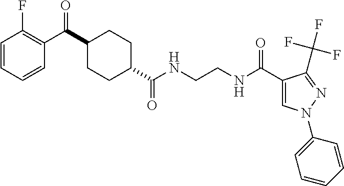 | N-(2-(trans- 4-(2- fluorobenzoyl) cyclohexane- carboxamido) ethyl)-1- phenyl-3- (trifluoro- methyl)-1H- pyrazole-4- carboxamide | ¹H NMR (400 MHz, CDCl₃) δ 1.38-1.52 (2H, m), 1.53-1.66 (2H, m), 1.96-2.19 (5H, m), 3.15 (1H, m), 3.47- 3.55 (2H, m), 3.56- 3.64 (2H, m), 6.19 (1H, t, J = 5.2 Hz), 6.81 (1H, br), 7.11 (1H, dd, J = 8.4, 11.2 Hz), 7.22 (1H, m), 7.42 (1H, t, J = 7.6 Hz), 7.46-7.55 (3H, m), 7.68-7.78 (3H, m), 8.41 (1H, s); m/z (APCI pos) 531.1 (100%) [M + H]. |
| E163 | 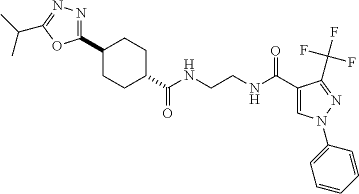 | N-(2-(trans- 4-(5- isopropyl- 1,3,4- oxadiazol-2- yl) cyclohexane- carboxamido) ethyl)-1- phenyl-3- (trifluoro- methyl)-1H- pyrazole-4- carboxamide | ¹H NMR (400 MHz, CDCl₃) δ 1.36 (6H, d, J = 6.8 Hz), 1.53-1.69 (4H, m), 1.96-2.08 (2H, m), 2.12-2.28 (3H, m), 2.85 (1H, m), 3.14 (1H, m), 3.47-3.56 (2H, m), 3.56-3.65 (2H, m), 6.32 (1H, t, J = 5.2 Hz), 6.80 (1H, m), 7.42 (1H, t, J = 7.6 Hz), 7.52 (2H, t, J = 7.6 Hz), 7.71 (2H, d, J = 7.6 Hz), 8.42 (1H, d, J = 0.4 Hz); m/z (APCI pos) 519.2 (100%) [M + H]. |
| E169 | 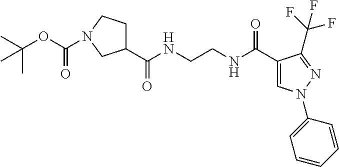 | tert-butyl 3- (2-(1-phenyl- 3- (trifluoro- methyl)-1H- pyrazole-4- carboxamido) ethyl carbamoyl) pyrrolidine-1- carboxylate | White solid (88%); ¹H NMR (400 MHz, DMSO-d₆) δ 1.36 (s, 9H), 1.85- 2.00 (m, 2H), 2.83- 2.94 (m, 1H), 3.10- 3.36 (m, 7H), 3.41 (dd, 1H, J = 10.5, 8.0 Hz), 7.45-7.50 (m, 1H), 7.58-7.65 (m, 2H), 7.78-7.82 (m, 2H), 8.06 (t, 1H, J = 5.6 Hz), 8.36 (m, 1H), 9.03 (s, 1H); m/,z (APCI pos) 396.2 (100%) [M + H − Boc]. |
| E170 | 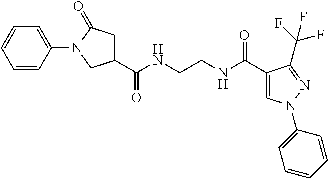 | N-(2-(5-oxo- 1- phenyl- pyrrolidine-3- carboxamido) ethyl)-1- phenyl-3- (trifluoro- methyl)-1H- pyrazole-4- carboxamide | White solid (67%); ¹H NMR (400 MHz, DMSO-d₆) δ 2.62-2.76 (m, 2H), 3.16-3.38 (m, 5H), 3.86 (dd, 1H, J = 9.7, 5.9 Hz), 3.96 (dd, 1H, J = 9.6, 8.7 Hz), 7.11 (m, 1H), 7.30-7.36 (m, 2H), 7.45-7.50 (m, 1H), 7.56-7.65 (m, 4H), 7.76-7.82 (m, 2H), 8.25 (t, 1H, J = 5.6 Hz), 8.39 (t, 1H, J = 5.6 Hz), 9.02 (s, 1H); m/z (APCI pos) 486.1 (100%) [M + H]. |
| E171 | 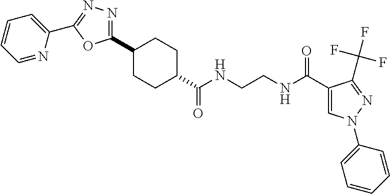 | 1-phenyl-N- (2-(trans-4- (5-(pyridin- 2-yl)-1,3,4- oxadiazol-2- yl) cyclohexane- carboxamido) ethyl)-3- (trifluoro- methyl)-1H- pyrazole-4- carboxamide | ¹H NMR (400 MHz, CDCl₃) δ 1.56-1.82 (4H, m), 2.02-2.11 (2H, m), 2.22 (1H, m), 2.29- 2.38 (2H, m), 3.02 (1H, m), 3.48-3.56 (2H, m), 3.58-3.66 (2H, m), 6.34 (1H, t, J = 4.8 Hz), 6.82 (1H, br), 7.39-7.49 (2H, m), 7.52 (2H, t, J = 7.6 Hz), 7.72 (2H, d, J = 7.6 Hz), 7.88 (1H, dt, J = 1.6, 7.6 Hz), 8.24 (1H, dd, J = 0.8, 7.6 Hz), 8.43 (1H, d, J = 0.8 Hz), 8.77 (1H, m); m/z (APCI pos) 554.2 (100%) [M + H]. |
| E174 | 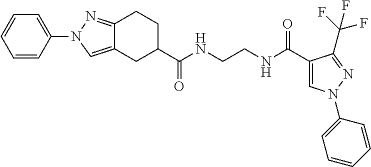 | 2-phenyl-N- (2-(1-phenyl- 3- (trifluoro- methyl)-1H- pyrazole-4- carboxamido) ethyl)- 4,5,6,7- tetrahydro- 2H-indazole- 5-carboxamide | Tan solid (70%); ¹H NMR (400 MHz, DMSO-d₆) δ 1.68-1.82 (m, 1H), 2.01-2.09 (m, 1H), 2.55-2.82 (m, 5H), 3.20-3.35 (m, 4H), 7.20-7.25 (m, 1H), 7.41-7.50 (m, 3H), 7.57-7.64 (m, 2H), 7.71-7.76 (m, 2H), 7.79-7.84 (m, 2H), 8.05 (t, 1H, J = 5.6 Hz), 8.17 (s, 1H), 8.40 (t, 1H, J = 5.6 Hz), 9.05 (s, 1H); m/z (APCI pos) 523.2 (100%) [M + H]. |
| E175 | 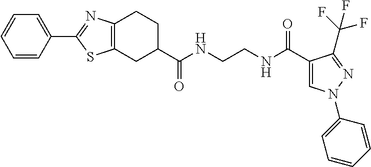 | 2-phenyl-N- (2-(1-phenyl- 3- (trifluoro- methyl)-1H- pyrazole-4- carboxamido) ethyl)- 4,5,6,7- tetrahydro- benzo[d] thiazole-6- carboxamide | White solid (78%); ¹H NMR (400 MHz, DMSO-d₆) δ 1.78-1.90 (m, 1H), 2.04-2.12 (m, 1H), 2.60-3.01 (m, 5H), 3.22-3.37 (m, 4H), 7.41-7.51 (m, 4H), 7.57-7.64 (m, 2H), 7.79-7.87 (m, 4H), 8.10 (t, 1H, J = 5.6 Hz), 8.40 (t, 1H, J = 5.6 Hz), 9.05 (s, 1H); m/z (APCI pos) 540.5 (100%) [M + H]. |
| E179 | 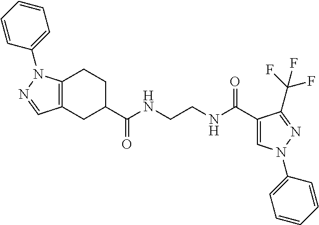 | 1-phenyl-N- (2-(1-phenyl- 3- (trifluoro- methyl)-1H- pyrazole-4- carboxamido) ethyl)- 4,5,6,7- tetrahydro- 1H-indazole- 5-carboxamide | White solid (39%); ¹H NMR (400 MHz, DMSO-d₆) δ 1.64-1.76 (m, 1H), 1.99-2.07 (m, 1H), 2.55-2.85 (m, 5H), 3.22-3.36 (m, 4H), 7.32-7.37 (m, 1H), 7.45-7.56 (m, 6H), 7.58-7.64 (m, 2H), 7.79-7.84 (m, 2H), 8.06 (t, 1H, J = 5.6 Hz), 8.40 (t, 1H, J = 5.6 Hz), 9.05 (s, 1H); m/z (APCI pos) 523.2 (100%) [M + H]. |
| E191 | 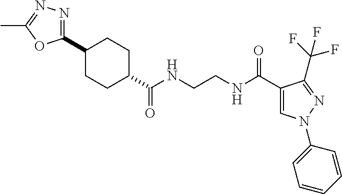 | N-(2-(trans- 4-(5-methyl- 1,3,4- oxadiazol-2- yl) cyclohexane- carboxamido) phenyl-3- (trifluoro- methyl)-1H- pyrazole-4- carboxamide | ¹H NMR (400 MHz, CDCl₃) δ 1.55-1.66 (m, 4H), 2.02-2.07 (m, 2H), 2.17-2.23 (m, 3H), 2.49 (s, 3H), 2.81- 2.85 (m, 1H), 3.49- 3.53 (m, 2H), 3.59- 3.63 (m, 2H), 6.29- 6.31 (m, 1H), 6.77 (br, 1H), 7.43 (t, J = 7.4 Hz, 1H), 7.52 (t, J = 7.9 Hz, 2H), 7.71 (d, J = 7.6 Hz, 2H), 8.42 (s, 1H); m/z (APCI pos) 491 [M + H]. |
| E192 | 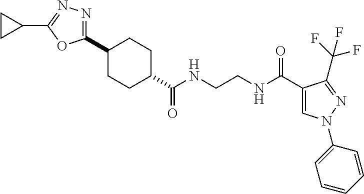 | N-(2-(trans- 4-(5- cyclopropyl- 1,3,4- oxadiazol-2- yl) cyclohexane- carboxamido) ethyl)-1- phenyl-3- (trifluoro- methyl)-1H- pyrazole-4- carboxamide | ¹H NMR (400 MHz, CDCl₃) δ 1.06-1.14 (m, 4H), 1.53-1.65 (m, 4H), 2.01-2.20 (m, 6H), 2.77-2.83 (m, 1H), 3.42-3.53 (m, 2H), 3.59-3.63 (m, 2H), 6.32 (t, J = Hz, 1H), 6.80 (br, 1H), 7.42 (t, J = 7.3 Hz, 1H), 7.52 (t, J = 8.00 Hz, 2H), 7.71 (d, J = 7.4 Hz, 2H), 8.42 (s, 1H); m/z (APCI pos) 517 [M + H]. |
| E194 | 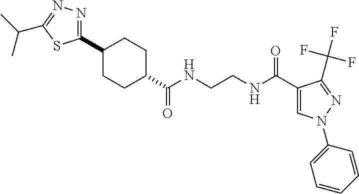 | N-(2-(trans- 4-(5- isopropyl- 1,3,4- thiadiazol-2- yl) cyclohexane- carboxamido) ethyl)-1- phenyl-3- (trifluoro- methyl)-1H- pyrazole-4- carboxamide | ¹H NMR (400 MHz, CDCl₃) δ 1.41 (6H, d, J = 6.8 Hz), 1.52-1.72 (4H, m), 2.00-2.08 (2H, m), 2.14-2.32 (3H, m), 3.10 (1H, m), 3.43 (1H, m), 3.48-3.56 (2H, m), 3.56-3.67 (2H, m), 6.32 (1H, t, J = 5.2 Hz), 6.82 (1H, m), 7.42 (1H, t, J = 7.6 Hz), 7.52 (2H, t, J = 7.6 Hz), 7.71 (2H, d, J = 7.6 Hz), 8.42 (1H, s); m/z (APCI pos) 535.1 (100%) [M + H]. |
| E195 | 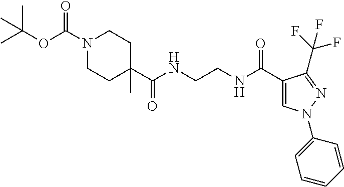 | tert-butyl 4- methyl-4-(2- (1-phenyl-3- (trifluoro- methyl)-1H- pyrazole-4- carboxamido) ethyl- carbamoyl) piperidine- 1-carboxylate | White solid (73%); ¹H NMR (400 MHz, DMSO-d₆) δ 1.8 (s, 3H), 1.19- 1.28 (m, 2H), 1.35 (s, 9H), 1.87-1.95 (m, 2H), 2.95-3.05 (m, 2H), 3.20-3.35 (m, 4H), 3.44-3.51 (m, 2H), 7.45-7.50 (m, 1H), 7.58-7.64 (m, 2H), 7.72 (t, 1H, J = 5.5 Hz), 7.78-7.82 (m, 2H), 8.35 (t, 1H, J = 5.5 Hz), 9.03 (s, 1H); m/z (APCI pos) 424.1 (100%) [M + H − Boc]. |
| E197 | 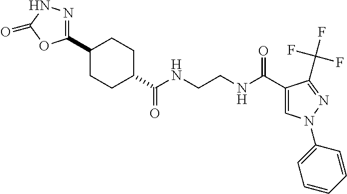 | N-(2-(trans- 4-(5-oxo-4,5- dihydro- 1,3,4- oxadiazol-2- yl) cyclohexane- carboxamido) ethyl)-1- phenyl-3- (trifluoro- methyl)-1H- pyrazole-4- carboxamide | ¹H NMR (400 MHz, DMSO- d₆) δ 1.28-1.52 (4H, m), 1.76-1.86 (2H, m), 1.92-2.02 (2H, m), 2.10 (1H, m), 2.57 (1H, m), 3.14-3.24 (2H, m), 3.24-3.34 (2H, m), 7.48 (1H, t, J = 7.6 Hz), 7.61 (2H, t, J = 7.6 Hz), 7.81 (2H, d, J = 7.6 Hz), 7.87 (1H, t, J = 5.6 Hz), 8.36 (1H, t, J = 5.6 Hz), 9.04 (1H, s), 12.04 (1H, s); m/z (APCI pos) 493.1 (100%) [M + H]. |
| E198 | 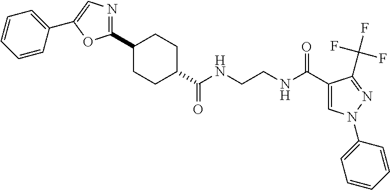 | 1-phenyl-N- (2-(trans-4- (5- phenyloxazol- 2- yl) cyclohexane- carboxamido) ethyl)-3- (trifluoro- methyl)-1H- pyrazole-4- carboxamide | ¹H NMR (400 MHz, CDCl₃) δ 1.54-1.74 (4H, m), 1.98-2.12 (2H, m), 2.20 (1H, m), 2.24- 2.34 (2H, m), 2.85 (1H, m), 3.48-3.56 (2H, m), 3.56-3.66 (2H, m), 6.27 (1H, t, J = 4.8 Hz), 6.83 (1H, br), 7.21 (1H, s), 7.30 (1H, t, J = 7.6 Hz), 7.36-7.46 (3H, m), 7.52 (2H, t, J = 7.6 Hz), 7.57-7.63 (2H, m), 7.69-7.75 (2H, m), 8.42 (1H, s); m/z (APCI pos) 552.2 (100%) [M + H]. |
| E204 | 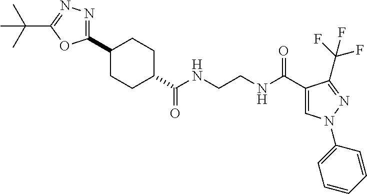 | N-(2-(trans- 4-(5-tert- butyl-1,3,4- oxadiazol-2- yl) cyclohexane- carboxamido) ethyl)-1- phenyl-3- (trifluoro- methyl)-1H- pyrazole-4- carboxamide | ¹H NMR (400 MHz, CDCl₃) δ 1.39 (s, 9H), 1.55- 1.67 (m, 4H), 1.89- 2.03 (m, 2H), 2.21- 2.23 (m, 3H), 2.84 (br, 1H), 3.49-3.53 (m, 2H), 3.59-3.63 (m, 2H), 6.45-6.46 (m, 1H), 6.89 (br, 1H), 7.40-7.44 (m, 1H), 7.49-7.53 (m, 2H), 7.70-7.23 (m, 2H), 8.43 (s, 1H); m/z (APCI pos) 533 [M + H]. |
| E214 | 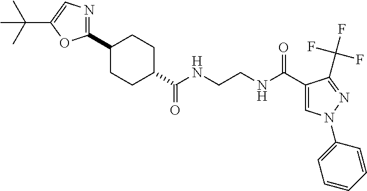 | N-(2-(trans- 4-(5-tert- butyloxazol- 2- yl) cyclohexane- carboxamido) ethyl)-1- phenyl-3- (trifluoro- methyl)-1H- pyrazole-4- carboxamide | ¹H NMR (400 MHz, CDCl₃) δ 1.26 (9H, s), 1.48- 1.66 (4H, m), 1.92- 2.08 (2H, m), 2.10- 2.28 (3H, m), 2.72 (1H, m), 3.46-3.56 (2H, m), 3.56-3.66 (2H, m), 6.27 (1H, t, J = 5.2 Hz), 6.53 (1H, s), 6.87 (1H, br), 7.42 (1H, t, J = 7.6 Hz), 7.52 (2H, t, J = 7.6 Hz), 7.71 (2H, d, J = 7.6 Hz), 8.41 (1H, d, J = 0.8 Hz); m/z (APCI pos) 532.2 (100%) [M + H]. |
| E215 | 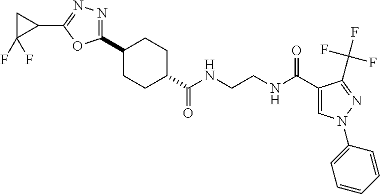 | N-(2-(trans- 4-(5-(2,2- difluoro- cyclopropyl)- 1,3,4- oxadiazol-2- yl) cyclohexane- carboxamido) ethyl)-1- phenyl-3- (trifluoro- methyl)-1H- pyrazole-4- carboxamide | ¹H NMR (400MHz, DMSO- d₆) δ 1.39-1.57 (4H, m), 1.76-1.90 (2H, m), 2.00-2.24 (4H, m), 2.31 (1H, m), 2.90 (1H, m), 3.16-3.24 (2H, m), 3.24-3.34 (2H, m), 3.44 (1H, m), 7.48 (1H, t, J = 7.6 Hz), 7.61 (2H, t, J = 7.6 Hz), 7.81 (2H, d, J = 7.6 Hz), 7.89 (1H, t, J = 5.6 Hz), 8.37 (1H, t, J = 5.6 Hz), 9.04 (1H, s); m/z (APCI pos) 553.1 (100%) [M + H]. |
| E216 | 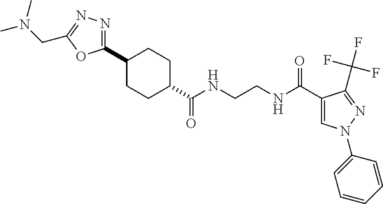 | N-(2-(trans- 4-(5- ((dimethyl- amino)methyl)- 1,3,4- oxadiazol-2- yl) cyclohexane- carboxamido) ethyl)-1- phenyl-3- (trifluoro- methyl)-1H- pyrazole-4- carboxamide | ¹H NMR (400 MHz, CDCl₃) δ 1.52-1.72 (4H, m), 1.96-2.10 (2H, m), 2.12-2.30 (3H, m), 2.34 (6H, s), 2.88 (1H, m), 3.46-3.56 (2H, m), 3.56-3.66 (2H, m), 3.71 (2H, s), 6.34 (1H, t, J = 4.8 Hz), 6.81 (1H, br), 7.42 (1H, m), 7.48- 7.56 (2H, m), 7.68- 7.74 (2H, m), 8.42 (1H, d, J = 0.8 Hz); m/z (APCI pos) 534.2 (100%) [M + H]. |
| E217 | 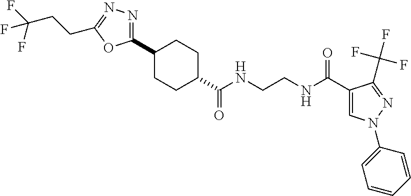 | 1-phenyl-3- (trifluoro- methyl)-N-(2- (trans-4-(5- (3,3,3- trifluoro- propyl)-1,3,4- oxadiazol-2- yl) cyclohexane- carboxamido) ethyl)-1H- pyrazole-4- carboxamide | ¹H NMR (400 MHz, CDCl₃) δ 1.52-1.68 (4H, m), 1.96-2.10 (2H, m), 2.12-2.28 (3H, m), 2.58-2.72 (2H, m), 2.86 (1H, m), 3.09 (2H, t, J = 8.0 Hz), 3.46-3.56 (2H, m), 3.56-3.66 (2H, m), 6.30 (1H, m), 6.75 (1H, br), 7.43 (1H, t, J = 7.6 Hz), 7.52 (2H, t, J = 7.6 Hz), 7.72 (2H, d, J = 7.6 Hz), 8.42 (1H, s); m/z (APCI pos) 573.1 (100%) [M + H]. |
| E222 | 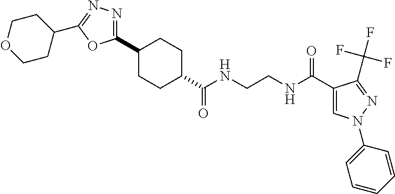 | 1-phenyl-N- (2-(trans-4- (5- (tetrahydro- 2H-pyran-4- yl)-1,3,4- oxadiazol-2- yl) cyclohexane- carboxamido) ethyl)-3- (trifluoro methyl)-1H- pyrazole-4- carboxamide | ¹H NMR (400 MHz, CDCl₃) δ 1.42-1.70 (4H, m), 1.86-2.10 (6H, m), 2.12-2.28 (3H, m), 2.86 (1H, m), 3.13 (1H, m), 3.46-3.66 (6H, m), 3.98-4.08 (2H, m), 6.32 (1H, t, J = 5.2 Hz), 6.77 (1H, br), 7.43 (1H, t, J = 8.0 Hz), 7.52 (2H, t, J = 8.0 Hz), 7.72 (2H, d, J = 8.0 Hz), 8.42 (1H, s); m/z (APCI pos) 561.2 (100%) [M + H]. |
| E223 | 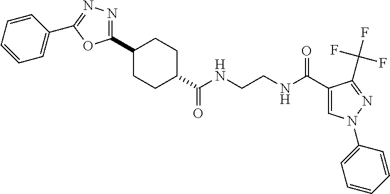 | 1-(5-phenyl- 1,3,4- oxadiazol-2- yl)-N-(2-(1- phenyl-3- (trifluoro- methyl)-1H- pyrazole-4- carboxamido) ethyl) piperidine-4- carboxamide | ¹H NMR (400 MHz, DMSO- d₆) δ 1.56-1.72 (2H, m), 1.76-1.86 (2H, m), 2.38 (1H, m), 3.06- 3.18 (2H, m), 3.18- 3.26 (2H, m), 3.26- 3.34 (2H, m), 3.97 (2H, d, J = 12.8 Hz), 7.47 (1H, t, J = 7.6 Hz), 7.50-7.56 (3H, m), 7.61 (2H, t, J = 7.6 Hz), 7.81 (2H, d, J = 7.6 Hz), 7.84-7.92 (2H, m), 7.98 (1H, t, J = 5.6 Hz), 8.37 (1H, t, J = 5.6 Hz), 9.04 (1H, s); m/z (APCI pos) 554.3 (100%) [M + H]. |
| E224 | 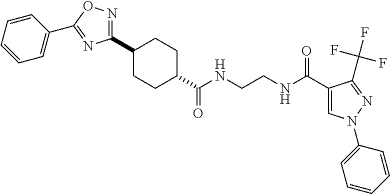 | 1-phenyl-N- (2-(trans-4- (5-phenyl- 1,2,4- oxadiazol-3- yl) cyclohexane- carboxamido) ethyl)-3- (trifluoro- methyl)-1H- pyrazole-4- carboxamide | ¹H NMR (400 MHz, CDCl₃) δ 1.56-1.76 (4H, m), 1.98-2.12 (2H, m), 2.16-2.30 (3H, m), 2.86 (1H, m), 3.48- 3.57 (2H, m), 3.57- 3.66 (2H, m), 6.31 (1H, br), 6.89 (1H, br), 7.42 (1H, t, J = 7.6 Hz), 7.48-7.62 (5H, m), 7.68-7.76 (2H, m), 8.11 (2H, d, J = 7.2 Hz), 8.43 (1H, s); m/z (APCI pos) 553.1 (100%) [M + H]. |
| E235 | 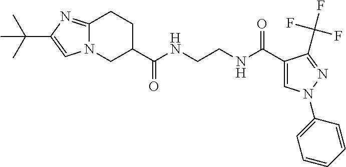 | 2-tert-butyl- 2-(2-(1- phenyl-3- (trifluoro- methyl)-1H- pyrazole-4- carboxamido) ethyl)- 5,6,7,8- tetrahydro- imidazo[1,2- a]pyridine-6- carboxamide | ¹H NMR (400 MHz, CDCl₃) δ 1.25 (s, 9H), 1.99- 2.10 (m, 1H), 2.18- 2.22 (m, 1H), 2.74- 2.85 (m, 2H), 2.98- 3.05 (m, 1H), 3.51- 3.58 (m, 2H), 3.60- 3.68 (m, 2H), 4.06 (d, J = 8.0 Hz, 2H), 6.47 (s, 1H), 6.72 (br, 2H), 7.43 (t, J = 7.4 Hz, 1H), 7.52 (t, J = 7.8 Hz, 2H), 7.71 (d, J = 7.4 Hz, 2H), 8.49 (s, 1H); m/z (APCI pos) 503 [M + H]. |
| E236 | 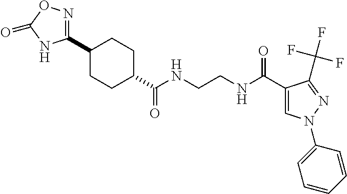 | N-(2-(trans- 4-(5-oxo-4,5- dihydro- 1,2,4- oxadiazol-3- yl) cyclohexane- carboxamido) ethyl)-1- phenyl-3- (trifluoro- methyl)-1H- pyrazole-4- carboxamide | ¹H NMR (400 MHz, DMSO- d₆) δ 1.32-1.50 (4H, m), 1.74-1.88 (2H, m), 1.90-2.02 (2H, m), 2.10 (1H, m), 2.50 (1H, m), 3.14-3.36 (4H, m), 7.48 (1H, t, J = 7.6 Hz), 7.61 (2H, t, J = 7.6 Hz), 7.81 (2H, d, J = 8.0 Hz), 7.89 (1H, t, J = 5.6 Hz), 8.36 (1H, t, J = 5.6 Hz), 9.04 (1H, s), 12.17 (1H, s); m/z (APCI pos) 493.1 (100%) [M + H]. |

### Example E42

### 1-(Cyclopropylsulfonyl)-N-(2-(1-phenyl-3-(trifluoromethyl)-1H-pyrazole-4-carboxamido)ethyl)piperidine-4-carboxamide

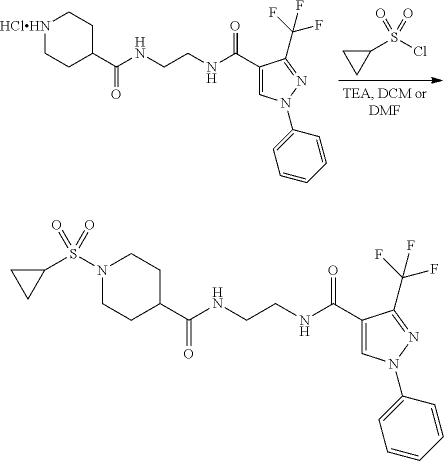

To N-(2-(1-phenyl-3-(trifluoromethyl)-1H-pyrazole-4-carboxamido)ethyl)piperidine-4-carboxamide hydrochloride (200 mg, 0.45 mmol) in DCM (5 mL) and triethylamine (113 mg, 1.1 mmol) was added cyclopropanesulfonyl chloride (63 mg, 0.45 mmol). The mixture was stirred for 18 hours then washed with water and brine, dried and concentrated to dryness. The residue was crystallized from AcOEt (46 mg, 29%): ¹H NMR (400 MHz, DMSO-d₆) δ 0.88-0.98 (m, 4H), 1.50-1.63 (m, 2H), 1.75-1.83 (m, 2H), 2.24 (m, 1H), 2.55 (m, 1H), 2.81 (m, 2H), 3.17-3.32 (m, 4H), 3.55-3.63 (m, 2H), 7.48 (m, 1H), 7.60 (m, 2H), 7.77-7.83 (m, 2H), 7.95 (t, 1H, J=5.5 Hz), 8.37 (t, 1H, J=5.5 Hz), 9.04 (s, 1H); m/z (APCI pos) 514.1 (100%) [M+H].

Compounds of the following structures were prepared from the corresponding sulfonyl chlorides and amines in DCM or DMF, using a similar method to that described above.

| Ex # | Structure | Name | Physical Data |
|---|---|---|---|
| E43 | 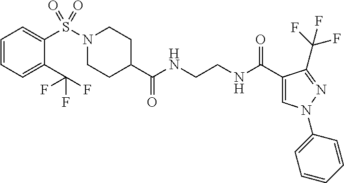 | N-(2-(1- phenyl-3- (trifluoro- methyl)-1H- pyrazole-4- carboxamido) ethyl)-1-(2- (trifluoro- methyl) phenyl- sulfonyl) piperidine-4- carboxamide | White solid (49%); ¹H NMR (400 MHz, DMSO-d₆) δ 1.46-1.60 (m, 2H), 1.73-1.81 (m, 2H), 2.24 (m, 1H), 2.77 (m, 2H), 3.16-3.30 (m, 4H), 3.68 (m, 2H), 7.48 (m, 1H), 7.61 (m, 2H), 7.80 (m, 2H), 7.85-7.95 (m, 3H), 8.00-8.06 (m, 2H), 8.35 (t, 1H, J = 5.5 Hz), 9.04 (s, 1H); m/z (APCI pos) 618.1 (100%) [M + H] |
| E44 | 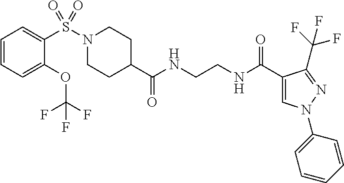 | N-(2-(1- phenyl-3- (trifluoro- methyl)-1H- pyrazole-4- carboxamido) ethyl)-1-(2- (trifluoro- methoxy) phenyl- sulfonyl) piperidine-4- carboxamide | White solid (54%); ¹H NMR (400 MHz, DMSO-d₆) δ 1.44-1.56 (m, 2H), 1.72-1.80 (m, 2H), 2.19 (m, 1H), 2.65 (m, 2H), 3.14-3.30 (m, 4H), 3.68 (m, 2H), 7.48 (m, 1H), 7.57- 7.64 (m, 4H), 7.78- 7.84 (m, 3H), 8.87- 8.96 (m, 2H), 8.34 (t, 1H, J = 5.5 Hz), 9.02 (s, 1H); m/z (APCI pos) 634.1 (100%) [M + H]. |
| E51 | 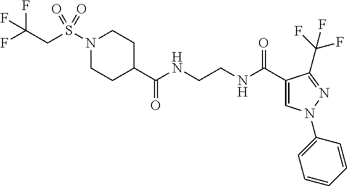 | N-(2-(1- phenyl-3- (trifluoro- methyl)-1H- pyrazole-4- carboxamido) ethyl)-1- (2,2,2- trifluoro- ethylsulfonyl) piperidine-4- carboxamide | White solid (67%); ¹H NMR (400 MHz, DMSO-d₆) δ 1.48-1.60 (m, 2H), 1.75-1.83 (m, 2H), 2.24 (m, 1H), 2.82- 2.90 (m, 2H), 3.18- 3.32 (m, 4H), 3.60- 3.66 (m, 2H), 4.47 (q, 2H, J = 10.1 Hz), 7.48 (m, 1H), 7.61 (m, 2H), 7.80 (m, 2H), 7.96 (t, 1H, J = 5.5 Hz), 8.37 (t, 1H, J = 5.5 Hz), 9.04 (s, 1H); m/z (APCI pos) 556.0 (100%) [M + H]. |
| E52 | 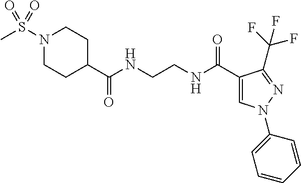 | 1- (methyl- sulfonyl)- N-(2-(1- phenyl-3- (trifluoro- methyl)-1H- pyrazole-4- carboxamido) ethyl) piperidine-4- carboxamide | White solid (80%); ¹H NMR (400 MHz, DMSO-d₆) δ 1.50-1.62 (m, 2H), 1.75-1.83 (m, 2H), 2.21 (m, 1H), 2.65- 2.74 (m, 2H), 2.84 (s, 3H), 3.18-3.30 (m, 4H), 3.50-3.56 (m, 2H), 7.48 (m, 1H), 7.61 (m, 2H), 7.80 (m, 2H), 7.96 (t, 1H, J = 5.5 Hz), 8.37 (t, 1H, J = 5.5 Hz), 9.04 (s, 1H); m/z (APCI pos) 488.1 (100%) [M + H]. |
| E53 | 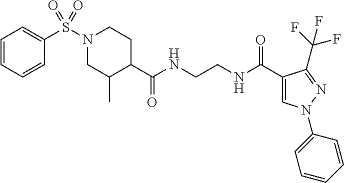 | 3-methyl-N- (2-(1-phenyl- 3- (trifluoro- methyl)-1H- pyrazole-4- carboxamido) ethyl)-1- (phenyl- sulfonyl) piperidine-4- carboxamide | White solid (77%); ¹H NMR (400 MHz, DMSO-d₆) δ 0.85 (d, 3H, J = 9.1 Hz), 1.56-1.65 (m, 1H), 1.73-1.83 (m, 1H), 2.03-2.12 (m, 1H), 2.23-2.28 (m, 1H), 2.57-2.70 (m, 2H), 3.12-3.27 (m, 6H), 7.48 (m, 1H), 7.58-7.65 (m, 4H), 7.68-7.73 (m, 3H), 7.78-7.82 (m, 2H), 7.85 (t, 1H, J = 5.5 Hz), 8.32 (t, 1H, J = 5.5 Hz), 9.03 (s, 1H); m/z (APCI pos) 564.1 (100%) [M + H]. |
| E73 | 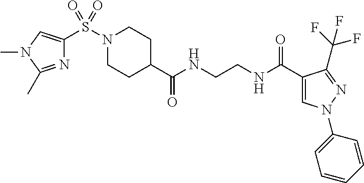 | 1-(1,2- dimethyl-1H- imidazol-4- ylsulfonyl)- N-(2-(1- phenyl-3- (trifluoro- methyl)-1H- pyrazole-4- carboxamido) ethyl) piperidine-4- carboxamide | White solid (32%); ¹H NMR (400 MHz, DMSO-d₆) δ 1.49-1.60 (m, 2H), 1.72-1.79 (m, 2H), 2.04-2.12 (m, 1H), 2.29 (s, 3H), 2.42- 2.48 (m, 2H), 3.15- 3.30 (m, 4H), 3.52- 3.58 (m, 2H), 3.60 (s, 3H), 7.45-7.51 (m, 1H), 7.58-7.64 (m, 2H), 7.70 (s, 1H), 7.78-7.82 (m, 2H), 7.85 (t, 1H, J = 5.6 Hz), 8.36 (t, 1H, J = 5.6 Hz), 9.03 (s, 1H); m/z (APCI pos) 568.1 (100%) [M + H]. |
| E74 | 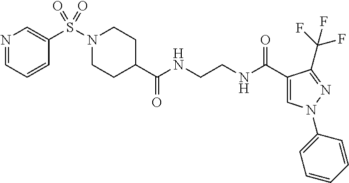 | N-(2-(1- phenyl-3- (trifluoro- methyl)-1H- pyrazole-4- carboxamido) ethyl)-1- (pyridin-3- ylsulfonyl) piperidine-4- carboxamide | White solid (8%); ¹H NMR (400 MHz, DMSO-d₆) δ 1.48-1.60 (m, 2H), 1.73-1.80 (m, 2H), 2.07-2.14 (m, 1H), 2.32-2.42 (m, 2H), 3.13-3.28 (m, 4H), 3.60-3.68 (m, 2H), 7.46-7.51 (m, 1H), 7.58-7.64 (m, 2H), 7.66-7.70 (m, 1H), 7.78-7.82 (m, 2H), 7.86 (t, 1H, J = 5.6 Hz), 8.14-8.18 (m, 1H), 8.34 (t, 1H, J = 5.5 Hz), 8.87-8.92 (m, 2H), 9.02 (s, 1H); m/z (APCI pos) 551.1 (100%) [M + H]. |
| E98 | 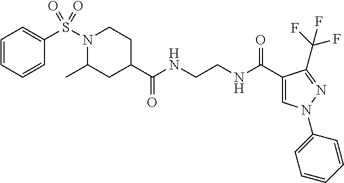 | 2-methyl-N- (2-(1-phenyl- 3- (trifluoro- methyl)-1H- pyrazole-4- carboxamido) ethyl)-1- (phenyl- sulfonyl) piperidine-4- carboxamide | White solid (33%); ¹H NMR (400 MHz, DMSO-d₆) δ 1.18 (d, 3H, J = 6.6 Hz), 1.66-1.92 (m, 4H), 2.22 (m, 1H), 3.20 (m, 1H), 3.44- 3.60 (m, 5H), 3.70 (m, 1H), 6.35 (m, 1H), 6.66 (m, 1H), 7.41- 7.58 (m, 6H), 7.72 (m, 2H), 7.79 (m, 2H), 8.41 (m, 1H); m/z (APCI pos) 564.1 (100%) [M + H]. |
| E173 | 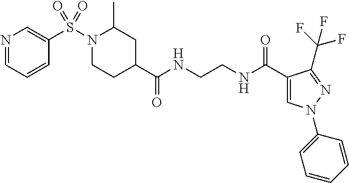 | 2-methyl-N- (2-(1-phenyl- 3- (trifluoro- methyl)-1H- pyrazole-4- carboxamido) ethyl)-1- (pyridin-3- ylsulfonyl) piperidine-4- carboxamide | Solid (9%); ¹H NMR (400 MHz, DMSO-d₆) δ 1.11 (d, 3H, J = 6.6 Hz), 1.61 (m, 2H), 1.72 (m, 2H), 2.21 (m, 1H), 3.14-3.28 (m, 5H), 3.47 (m, 1H), 3.61 (m, 1H), 7.48 (m, 1H), 7.63 (m, 3H), 7.81 (m, 2H), 7.90 (m, 1H), 8.21 (m, 1H), 8.34 (m, 1H), 8.84 (m, 1H), 8.96 (m, 1H), 9.02 (m, 1H); m/z (APCI pos) 565.1 (100%) [M + H]. |
| E178 | 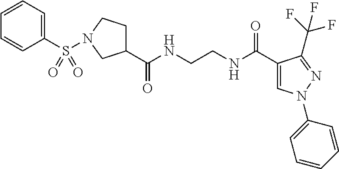 | 1-phenyl-N- (2-(1- (phenyl- sulfonyl) pyrrolidine-3- carboxamido) ethyl)-3- (trifluoro- methyl)-1H- pyrazole-4- carboxamide | White solid (70%); ¹H NMR (400 MHz, DMSO-d₆) δ 1.71-1.81 (m, 1H), 1.85-1.95 (m, 1H), 2.73-2.82 (m, 1H), 3.09-3.27 (m, 7H), 3.38-3.44 (m, 1H), 7.45-7.50 (m, 1H), 7.58-7.65 (m, 4H), 7.68-7.73 (m, 1H), 7.76-7.83 (m, 4H), 8.04 (t, 1H, J = 5.6 Hz), 8.34 (t, 1H, J = 5.6 Hz), 9.01 (s, 1H); m/z (APCI pos) 536.1 (100%) [M + H]. |

### Example E47

### 1-(3,3-Dimethylbutanoyl)-3-methyl-N-(2-(1-phenyl-3-(trifluoromethyl)-1H-pyrazole-4-carboxamido)ethyl)piperidine-4-carboxamide

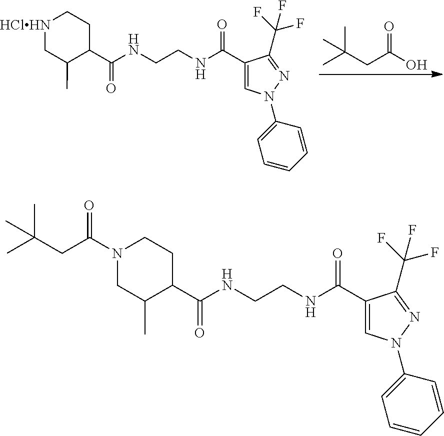

To 3-methyl-N-(2-(1-phenyl-3-(trifluoromethyl)-1H-pyrazole-4-carboxamido)ethyl)piperidine-4-carboxamide hydrochloride (100 mg, 0.22 mmol) in DCM (50 mL) were successively added 3,3-dimethylbutanoic acid (25 mg, 0.22 mmol), EDAC.HCl (54 mg, 0.28 mmol), HOBt.H₂O (41 mg, 0.3 mmol) and triethylamine (44 mg, 0.43 mmol). The mixture was stirred at room temperature for 18 hours then DCM was added (30 mL). The organic layer was successively washed with 1N HCl (50 mL), 10% potassium carbonate aqueous solution (50 mL) and brine (2×50 mL). The organic layer was dried over MgSO₄ then concentrated to yield a white solid. The crude solid was purified by MPLC (DCM/MeOH 99/1) to yield 1-(3,3-dimethylbutanoyl)-3-methyl-N-(2-(1-phenyl-3-(trifluoromethyl)-1H-pyrazole-4-carboxamido)ethyl)piperidine-4-carboxamide as a white solid (96 mg, 84%). Crystallized from AcOEt: ¹H NMR (400 MHz, DMSO-d₆) δ 0.73 (dd, 3H, J=6.9, 24.6 Hz), 0.96 (s, 9H), 1.40-1.75 (m, 2H), 2.00-2.15 (m, 1H), 2.45 (m, 1H), 2.90-2.96 (m, 1H), 3.05-3.13 (m, 1H), 3.18-3.30 (m, 4H), 3.65-4.12 (m, 2H), 5.75 (m, 2H), 7.48 (m, 1H), 7.60 (m, 2H), 7.77-7.90 (m, 3H), 8.35 (m, 1H), 9.04 (s, 1H); m/z (APCI pos) 522.2 (100%) [M+H].

Compounds of the following structures were prepared from the corresponding acids and amines, using a similar method to that described above.

| Ex # | Structure | Name | Physical Data |
|---|---|---|---|
| E48 | 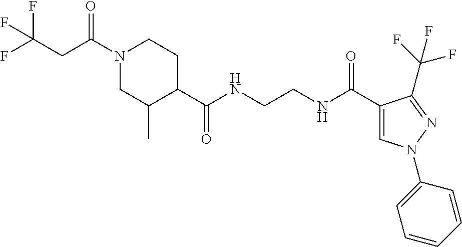 | 3-methyl-N- (2-(1-phenyl- 3- (trifluoromethyl)- 1H- pyrazole-4- carboxamido) ethyl)-1- (3,3,3- trifluoropropanyl) piperidine- 4- carboxamide | White solid (75%); ¹H NMR (400 MHz, DMSO-d₆) δ 0.75 (dd, 3H, J = 6.9, 24.6 Hz), 1.45- 1.55 (m, 1H), 1.60- 1.82 (m, 1H), 2.05 (m, 1H), 2.47 (m, 1H), 3.01-3.30 (m, 6H), 3.45-3.85 (m, 3H), 3.89-3.98 (m, 1H), 7.48 (m, 1H), 7.60 (m, 2H), 7.78-7.83 (m, 2H), 7.90 (t, 1H), J = 5.5 Hz), 8.35 (t, 1H, J = 5.5 Hz), 9.05 (s, 1H); m/z (APCI pos) 534.1 (100%) [M + H]. |
| E89 | 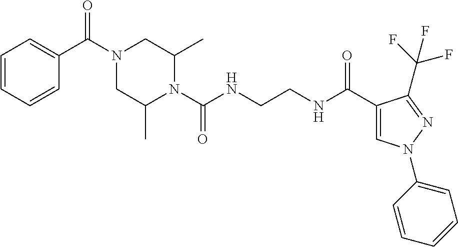 | 4-benzoyl- 2,6-dimethyl- N-(2-(1- phenyl-3- (trifluoromethyl)- 1H- pyrazole-4- carboxamido) ethyl)piperazine- 1- carboxamide | Solid (9%); ¹H NMR (400 MHz, DMSO-d₆) δ 1.26 (m, 8H), 1.61 (m, 2H), 3.51 (m, 2H), 3.59 (m, 2H), 4.12 (s, 2H), 5.39 (m, 1H), 7.04 (m, 1H), 7.42 (m, 6H), 7.51 (m, 2H), 7.70 (m, 2H), 8.41 (s, 1H); m/z (APCI pos) 543.1 (100%) [M + H]. |
| E97 | 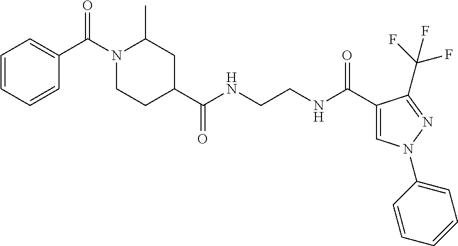 | 1-benzoyl-2- methyl-N-(2- (1-phenyl-3- (trifluoromethyl)- 1H- pyrazole-4- carboxamido) ethyl)piperidine- 4- carboxamide | Solid (61%); ¹H NMR (400 MHz, d₆-DMSO) δ 1.22 (d, 3H, J = 6.64 Hz), 1.75-2.04 (m, 3H), 2.48 (m, 1H), 2.69 (m, 1H), 3.29 (m, 1H), 3.49 (m, 2H), 3.58 (m, 2H), 3.73 (m, 1H), 4.35 (m, 1H), 6.79 (m, 1H), 6.94 (m, 1H), 7.39 (m, 6H), 7.51 (m, 2H), 7.71 (m, 2H), 8.50 (m, 1H); m/z (APCI pos) 528.2 (100%) [M + H]. |

### Example E57

### Ethyl 4-(2-(1-phenyl-3-(trifluoromethyl)-1H-pyrazole-4-carboxamido)ethylcarbamoyl)piperidine-1-carboxylate

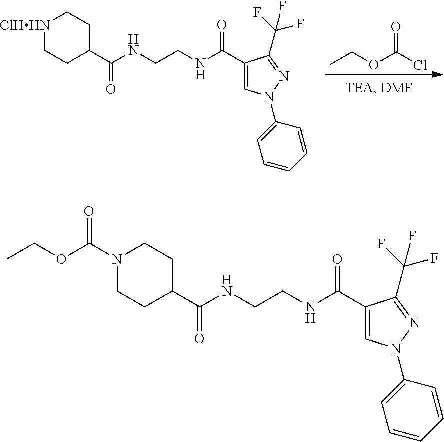

To N-(2-(1-phenyl-3-(trifluoromethyl)-1H-pyrazole-4-carboxamido)ethyl)piperidine-4-carboxamide hydrochloride (200 mg, 0.45 mmol) in DMF (5 mL) was added triethylamine (91 mg, 0.89 mmol) followed by ethyl chloroformate (49 mg, 0.45 mmol). The mixture was stirred at room temperature for 18 hours then concentrated to dryness. The residue was purified by MPLC (DCM/MeOH 95/5) then crystallized from AcOEt/MeOH to yield ethyl 4-(2-(1-phenyl-3-(trifluoromethyl)-1H-pyrazole-4-carboxamido)ethylcarbamoyl)piperidine-1-carboxylate as a white powder (42 mg, 20%): ¹H NMR (400 MHz, DMSO-d₆) δ 1.16 (t, 3H, J=7.1 Hz), 1.33-1.46 (m, 2H), 1.63-1.70 (m, 2H), 2.23-2.34 (m, 1H), 2.77 (br, 2H), 3.17-3.31 (m, 4H), 3.91-3.97 (m, 2H), 4.01 (q, 2H, J=7.1 Hz), 7.48 (m, 1H), 7.58-7.64 (m, 2H), 7.79-7.83 (m, 2H), 7.91 (t, 1H, J=5.5 Hz), 8.36 (t, 1H, J=5.5 Hz), 9.03 (s, 1H); m/z (APCI pos) 482.1 (100%) [M+H].

Compounds of the following structures were prepared from the corresponding chloroformates and amines, using a similar method to that described above.

| Ex # | Structure | Name | Physical Data |
|---|---|---|---|
| E58 | 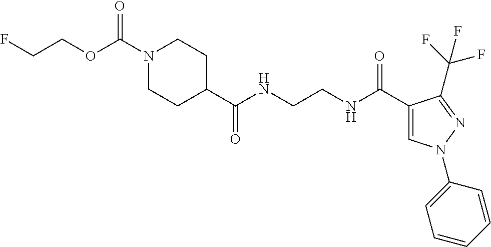 | 2-fluoroethyl 4-(2-(1- phenyl-3- (trifluoromethyl)- 1H- pyrazole-4- carboxamido) ethylcarbamoyl) piperidine- 1-carboxylate | White solid (30%); ¹H NMR (400 MHz, DMSO-d₆) δ 1.36-1.48 (m, 2H), 1.65-1.73 (m, 2H), 2.25-2.34 (m, 1H), 2.83 (br, 2H), 3.17- 3.31 (m, 4H), 3.93- 4.00 (m, 2H), 4.17- 4.21 (m, 1H), 4.25- 4.28 (m, 1H), 4.52 (t, 1H, J = 4.0 Hz), 4.65 (t, 1H, J = 4.0 Hz), 7.48 (m, 1H), 7.58- 7.64 (m, 2H), 7.79- 7.83 (m, 2H), 7.93 (t, 1H, J = 5.5 Hz), 8.36 (t, 1H, J = 5.5 Hz), 9.03 (s, 1H); m/z (APCI pos) 500.1 (100%) [M + H]. |
| E59 | 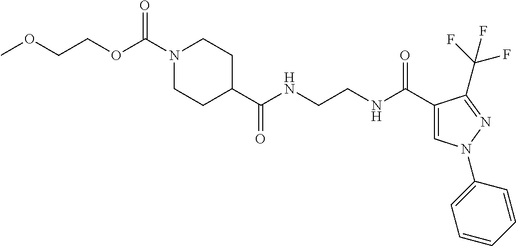 | 2- methoxyethyl 4-(2-(1- phenyl-3- (trifluoromethyl)- 1H- pyrazole-4- carboxamido) ethylcarbamoyl) piperidine- 1-carboxylate | White solid (31%); ¹H NMR (400 MHz, DMSO-d₆) δ 1.35-1.47 (m, 2H), 1.64-1.72 (m, 2H), 2.24-2.34 (m, 1H), 2.79 (br, 2H), 3.17- 3.31 (m, 7H), 3.50 (t, 2H, J = 4.7 Hz), 3.91- 3.97 (m, 2H), 4.09 (t, 2H, J = 4.7 Hz, 7.48 (m, 1H), 7.58-7.64 (m, 2H), 7.79-7.83 (m, 2H), 7.92 (t, 1H, J = 5.5 Hz), 8.36 (t, 1H, J = 5.5 Hz), 9.03 (s, 1H); m/z (APCI pos) 512.2 (100%) [M + H]. |
| E70 | 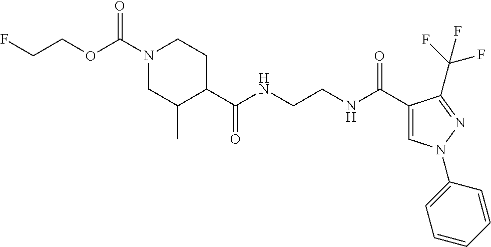 | 2-fluoroethyl 3-methyl-4- (2-(1-phenyl- 3- (trilfuoromethyl)- 1H- pyrazole-4- carboxamido) ethylcarbamoyl) piperidine- 1-carboxylate | White solid (93%); ¹H NMR (400 MHz, DMSO-d₆) δ 0.75 (d, 3H, J = 6.9 Hz), 1.44-1.52 (m, 1H), 1.63-1.74 (m, 1H), 2.0-2.08 (m, 1H), 2.40-2.46 (m, 1H), 2.90-3.30 (m, 6H), 3.63-3.71 (m, 1H), 3.80-3.88 (m, 1H), 4.15-4.29 (m, 2H), 4.49-4.67 (m, 2H), 7.48 (m, 1H), 7.58- 7.64 (m, 2H), 7.78- 7.84 (m, 2H), 7.89 (t, 1H, J = 5.5 Hz), 8.36 (t, 1H, J = 5.5 Hz), 9.04 (s, 1H); m/z (APCI pos) 514.1 (100%) [M + H]. |
| E71 | 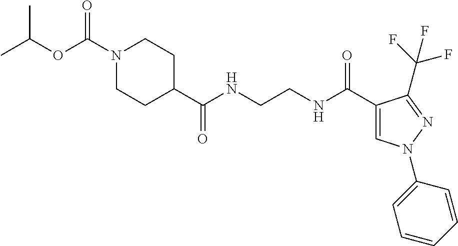 | isopropyl 4- (2-(1-phenyl- 3- (trifluoromethyl)- 1H- pyrazole-4- carboxamido) ethylcarbamoyl) piperidine- 1-carboxylate | White solid (83%); ¹H NMR (400 MHz, DMSO-d₆) δ 1.51 (s, 3H), 1.54 (s, 3H), 1.33-1.44 (m, 2H), 1.63-1.70 (m, 2H), 2.22-2.32 (m, 1H), 2.74 (br, 2H), 3.16-3.30 (m, 4H), 3.90-3.97 (m, 2H), 4.70-4.78 (m, 1H), 7.48 (m, 1H), 7.58- 7.64 (m, 2H), 7.79- 7.83 (m, 2H), 7.91 (t, 1H), J = 5.5 Hz), 8.35 (t, 1H, J = 5.5 Hz), 9.03 (s, 1H); m/z (APCI pos) 496.2 (80%) [M + H]. |
| E72 | 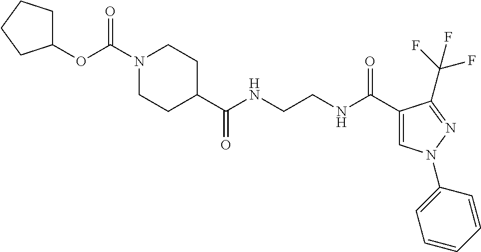 | cyclopentyl 4-(2-(1- phenyl-3- (trifluoromethyl)- 1H- pyrazole-4- carboxamido) ethylcarbamoyl) piperidine- 1-carboxylate | White solid (77%); ¹H NMR (400 MHz, DMSO-d₆) δ 1.32-1.80 (m, 12H), 2.22-2.32 (m, 1H), 2.74 (br, 2H), 3.16- 3.30 (m, 4H), 3.87- 3.97 (m, 2H), 4.93- 4.98 (m, 1H), 7.48 (m, 1H), 7.58-7.64 (m, 2H), 7.79-7.83 (m, 2H), 7.90 (t, 1H, J = 5.5 Hz), 8.35 (t, 1H, J = 5.5 Hz), 9.03 (s, 1H); m/z (APCI pos) 522.2 (50%) [M + H]. |
| E90 | 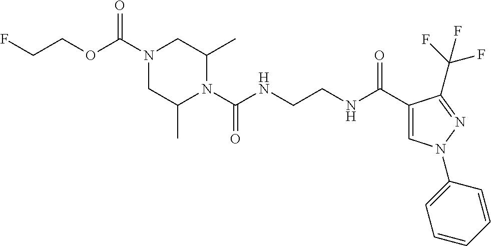 | 2-fluoroethyl 3,5-dimethyl- 4-((2-(1- phenyl-3- (trifluoromethyl)- 1H- pyrazole-4- carboxamido)ethyl) carbamoyl) piperazine- 1-carboxylate | Solid (15%); ¹H NMR (400 MHz, DMSO-d₆) δ 1.24 (d, 6H, J = 7.0 Hz), 3.05 (m, 2H), 3.51 (m, 2H), 3.58 (m, 2H), 4.03 (m, 4H), 4.38 (m, 2H), 4.55 (t, 1H, J = 4.3 Hz), 4.67 (t, 1H, J = 4.3 Hz), 5.38 (m, 1H), 7.18 (m, 1H), 7.42 (m, 1H), 7.51 (m, 2H), 7.71 (m, 2H), 8.44 (m, 1H); m/z (APCI pos) 529.1 (100%) [M + H]. |
| E99 | 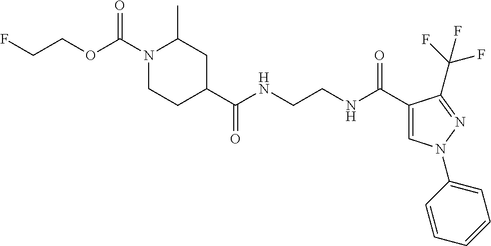 | 2-fluoroethyl 2-methyl-4- ((2-(1- phenyl-3- (trifluoromethyl)- 1H- pyrazole-4- carboxmido)ethyl) carbamoyl) piperidine- 1-carboxylate | Solid (72%); ¹H NMR (400 MHz, DMSO-d₆) δ 1.17 (d, 3H, J = 6.6 Hz), 1.71 (m, 1H), 1.87-1.98 (m, 3H), 2.37 (m, 1H), 3.20 (m, 1H), 3.51 (m, 2H), 3.60 (m, 2H), 3.86 (m, 1H), 4.06 (m, 1H), 4.28 (m, 1H), 4.35 (m, 1H), 4.53 (t, 1H, J = 4.7 Hz), 4.65 (t, 1H, J = 4.7 Hz), 6.33 (m, 1H), 6.69 (m, 1H), 7.43 (m, 1H), 7.52 (m, 2H), 7.72 (m, 2H), 8.42 (m, 1H); m/z (APCI pos) 514.1 (100%) [M + H]. |

### Example E76

### 3-Methyl-N-(2-(1-phenyl-3-(trifluoromethyl)-1H-pyrazole-4-carboxamido)ethyl)-4-(pyrrolidine-1-carbonyl)piperidine-1-carboxamide

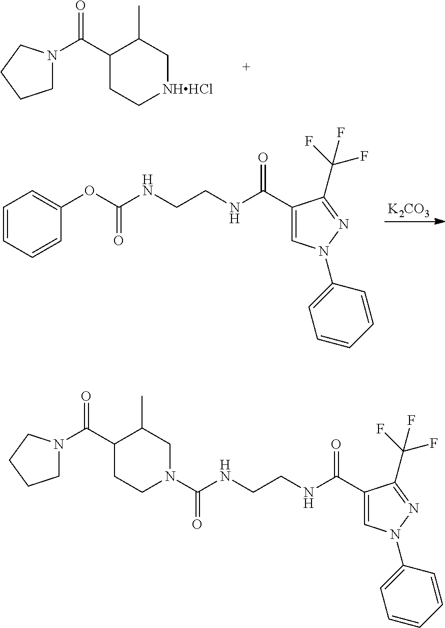

To (3-methylpiperidin-4-yl)(pyrrolidin-1-yl)methanone hydrochloride (80 mg, 0.34 mmol) in EtOH (1 mL) were added potassium carbonate (48 mg, 0.34 mmol) and phenyl 2-(1-phenyl-3-(trifluoromethyl)-1H-pyrazole-4-carboxamido)ethylcarbamate (144 mg, 0.34 mmol). The mixture was heated to 130° C. for 1 hour in a sealed tube then cooled down. Purification of the crude mixture by MPLC (DCM/MeOH 95/5) yielded 3-methyl-N-(2-(1-phenyl-3-(trifluoromethyl)-1H-pyrazole-4-carboxamido)ethyl)-4-(pyrrolidine-1-carbonyl)piperidine-1-carboxamide as a white solid (123 mg. 69%): ¹H NMR (400 MHz, DMSO-d₆) δ 0.74-0.80 (m, 3H), 1.20-1.40 (m, 2H), 1.70-2.00 (m, 6H), 2.70-3.30 (m, 8H), 3.40-3.52 (m, 2H), 3.64-3.80 (m, 2H), 6.54 (m, 1H), 7.48 (m, 1H), 7.58-7.64 (m, 2H), 7.78-7.84 (m, 2H), 8.40 (m, 1H), 9.03 (s, 1H); m/z (APCI pos) 521.0 (20%) [M+H], 197.1 (100%).

Compounds of the following structures were prepared from the corresponding amine salts, using a similar method to that described above with reaction temperatures ranging from 75° C. (refluxing ethanol) to 130° C. in opened or sealed vessels.

| Ex # | Structure | Name | Physical Data |
|---|---|---|---|
| E81 | 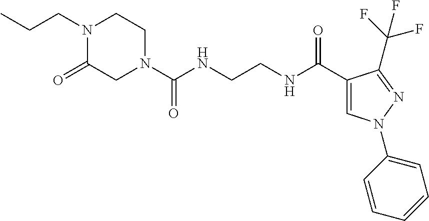 | 3-oxo-N-(2- (1-phenyl-3- (trifluoromethyl)- 1H- pyrazole-4- carboxamido)ethyl)- 4- propylpiperazine- 1- carboxamide | Solid (29%); ¹H NMR (400 MHz, DMSO-d₆) δ 0.81 (m, 3H), 1.46 (m, 2H), 3.17-3.30 (m, 8H), 3.53 (m, 2H), 3.90 (s, 2H), 6.75 (m, 1H), 7.48 (m, 1H), 7.61 (m, 2H), 7.82 (m, 2H), 8.38 (m, 1H), 9.05 (s, 1H); m/z (APCI pos) 467.0 (100%) [M + H]. |
| E82 | 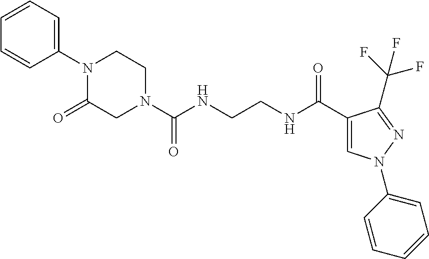 | 3-oxo-4- phenyl-N-(2- (1-phenyl-3- (trifluoromethyl)- 1H- pyrazole-4- carboxamido)ethyl) piperazine- 1-carboxamide | Solid (18%); ¹H NMR (400 MHz, DMSO-d₆) δ 3.22 (m, 2H), 3.34 (m, 2H), 3.70 (m, 4H), 4.11 (s, 2H), 6.83 (m, 1H), 7.24-7.49 (m, 6H), 7.60 (m, 2H), 7.82 (m, 2H), 8.41 (m, 1H), 9.06 (s, 1H); m/z (APCI pos) 501.0 (100%) [M + H]. |
| E83 | 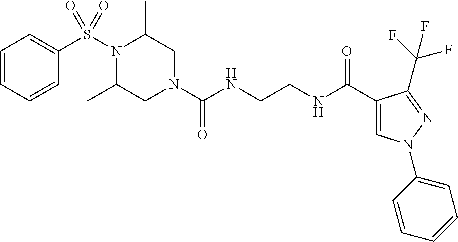 | 3,5-dimethyl- N-(2-(1- phenyl-3- (trifluoromethyl)- 1H- pyrazole-4- carboxamido)ethyl)- 4- (phenylsulfonyl) piperazine- 1- carboxamide | White solid (63%); ¹H NMR (400 MHz, DMSO-d₆) δ 1.18 (s, 3H), 1.20 (s, 3H), 2.53-2.56 (m, 1H), 3.15-3.30 (m, 4H), 3.70-3.75 (m, 2H), 3.95-4.07 (m, 3H), 6.59 (t, 1H, J = 5.3 Hz), 7.48 (m, 1H), 7.56-7.69 (m, 5H), 7.77-7.85 (m, 4H), 8.32 (t, 1H, J = 5.5 Hz), 9.01 (s, 1H); m/z (APCI pos) 579.0 (100%) [M + H]. |
| E86 | 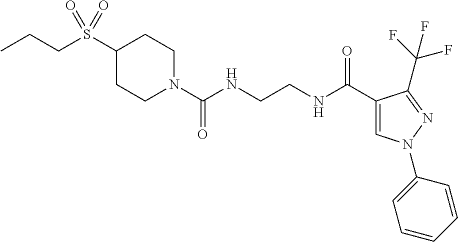 | N-(2-(1- phenyl-3- (trifluoromethyl)- 1H- pyrazole-4- carboxamido)ethyl)- 4- (propylsulfonyl) piperidine- 1- carboxamide | White solid (52%); ¹H NMR (400 MHz, DMSO-d₆) δ 0.99 (t, 3H, J = 7.4 Hz), 1.37-1.49 (m, 2H), 1.65-1.75 (m, 2H), 1.90-1.98 (m, 2H), 2.68-2.77 (m, 2H), 3.00-3.06 (m, 2H), 3.15-3.30 (m, 5H), 4.04-4.11 (m, 2H), 6.73 (t, 1H, J = 5.3 Hz), 7.48 (m, 1H), 7.58-7.64 (m, 2H), 7.78-7.84 (m, 2H), 8.40 (t, 1H, J = 5.5 Hz), 9.05 (s, 1H); m/z (APCI pos) 516.0 (100%) [M + H]. |
| E100 | 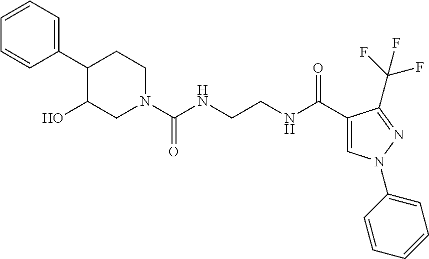 | 3-hydroxy-4- phenyl-N-(2- (1-phenyl-3- (trifluoromethyl)- 1H- pyrazole-4- carboxamido)ethyl) piperidine- 1- carboxamide | Solid (33%); ¹H NMR (400 MHz, d₆-DMSO) δ 1.52 (m, 1H), 1.66 (m, 1H), 2.45 (m, 2H), 2.69 (m, 1H), 3.21 (m, 2H), 3.30 (m, 2H), 3.45 (m, 1H), 3.96 (m, 1H), 4.19 (m, 1H), 4.70 (d, 1H, J = 6.25 Hz), 6.70 (m, 1H), 7.15-7.29 (m, 5H), 7.47 (m, 1H), 7.60 (m, 2H), 7.81 (m, 2H), 8.39 (m, 1H), 9.06 (m, 1H); m/z (APCI pos) 502.1 (100%) [M + H]. |
| E102 | 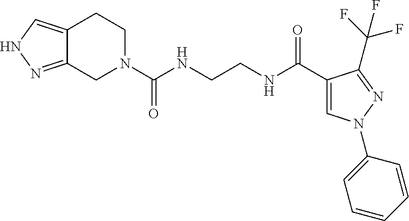 | N-(2-(1- phenyl-3- (trifluoromethyl)- 1H- pyrazole-4- carboxamido)ethyl)- 4,5- dihydro-2H- pyrazolo[3,4- c]pyridine- 6(7H)- carboxamide | White solid (25%); ¹H NMR (400 MHz, DMSO-d₆) δ 2.57-2.65 (m, 2H), 3.16-3.32 (m, 4H), 3.56-3.61 (m, 2H), 4.30-4.39 (m, 2H), 6.75 (t, 1H, J = 5.3 Hz), 7.45-7.50 (m, 2H), 7.58-7.64 (m, 2H), 7.78-7.84 (m, 2H), 8.41 (t, 1H, J = 5.5 Hz), 9.04 (s, 1H), 12.45 (s, 1H); m/z (APCI pos) 448.2 (100%) [M + H]. |
| E103 | 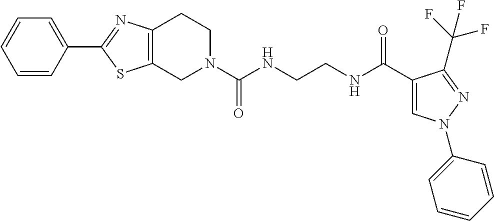 | 2-phenyl-N- (2-(1-phenyl- 3- (trifluoromethyl)- 1H- pyrazole-4- carboxamido)ethyl)- 6,7- dihydrothiazolo [5,4- c]pyridine- 5(4H)- carboxamide | White solid (29%); ¹H NMR (400 MHz, DMSO-d₆) δ 2.78-2.84 (m, 2H), 3.20-3.34 (m, 4H), 3.68-3.72 (m, 2H), 4.65 (s, 2H), 6.93 (t, 1H, J = 5.3 Hz), 7.45- 7.51 (m, 4H), 7.57- 7.63 (m, 2H), 7.77- 7.81 (m, 2H), 7.84- 7.88 (m, 2H), 8.40 (t, 1H, J = 5.5 Hz), 9.03 (s, 1H); m/z (APCI pos) 541.0 (100%) [M + H]. |
| E107 | 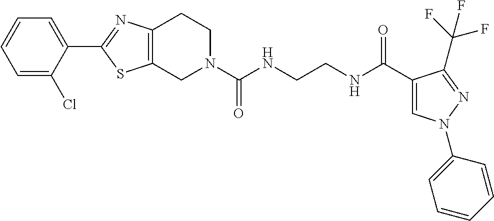 | 2-(2- chlorophenyl)- N-(2-(1- phenyl-3- (trifluoromethyl)- 1H- pyrazole-4- carboxamido) ethyl)-6,7- dihydrothiazolo [5,4- c]pyridine- 5(4H)- carboxamide | White solid (41%); ¹H NMR (400 MHz, DMSO-d₆) δ 2.82-2.87 (m, 2H), 3.20-3.34 (m, 4H), 3.68-3.74 (m, 2H), 4.69 (s, 2H), 6.95 (t, 1H, J = 5.3 Hz), 7.45- 7.51 (m, 3H), 7.57- 7.63 (m, 3H), 7.75- 7.81 (m, 2H), 8.08- 8.12 (m, 1H), 8.40 (t, 1H, J = 5.5 Hz), 9.03 (s, 1H); m/z (APCI pos) 575.1 (100%) [M]. |
| E108 | 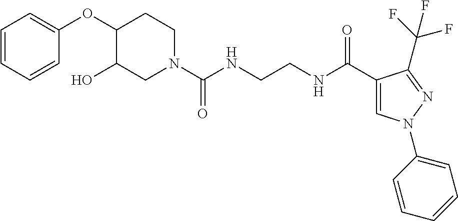 | 3-hydroxy-4- phenoxy-N-(2- (1-phenyl-3- (trifluoromethyl)- 1H- pyrazole-4- carboxamido)ethyl) piperidine- 1-carboxamide | Solid (1%); ¹H NMR (400 MHz, CD₃OD) δ 1.54 (m, 1H), 2.11 (m, 1H), 3.12-.326 (m, 2H), 3.35-3.40 (m, 2H), 3.47 (m, 2H), 3.67 (m, 2H), 3.87 (m, 1H), 4.27 (m, 1H), 6.94 (m, 3H), 7.25 (m, 2H), 7.44 (m, 1H), 7.55 (m, 2H), 7.82 (m, 2H), 8.74 (m, 1H); m/z (APCI pos) 518.1 (100%) [M + H]. |
| E113 | 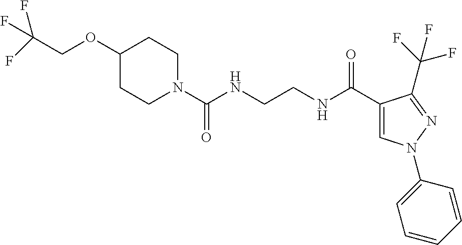 | N-(2-(1- phenyl-3- (trifluoromethyl)- 1H- pyrazole-4- carboxamido)ethyl)- 4- (2,2,2- trifluoroethoxy) piperidine- 1- carboxamide | White solid (23%); ¹H NMR (400 MHz, DMSO-d₆) δ 1.27-1.40 (m, 2H), 1.76-1.84 (m, 2H), 2.91-2.99 (m, 2H), 3.14-3.32 (m, 4H), 3.60-3.69 (m, 3H), 4.06 (q, 2H, J = 9.4 Hz), 6.65 (t, 1H, J = 5.4 Hz), 7.44-7.50 (m, 1H), 7.57-7.63 (m, 2H), 7.80-7.85 (m, 2H), 8.39 (t, 1H, J = 5.5 Hz), 9.06 (s, 1H); m/z (APCI pos) 508.2 (100%) [M + H]. |
| E162 | 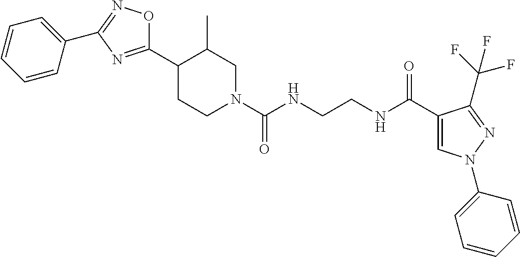 | 3-methyl-4- (3-phenyl- 1,2,4- oxadiazol-5- yl)-N-(2-(1- phenyl-3- (trifluoromethyl)- 1H- pyrazole-4- carboxamido)ethyl) piperidine- 1- carboxamide | White solid (66%); ¹H NMR (400 MHz, DMSO-d₆) δ 0.75 (d, 3H, J = 6.9 Hz), 1.86-1.96 (m, 2H), 2.22-2.29 (m, 1H), 3.10-3.33 (m, 6H), 3.50 (m, 1H), 3.61 (dd, 1H, J = 13.2, 5.4 Hz), 3.85 (m, 1H), 6.68 (t, 1H, J = 5.3 Hz), 7.44-7.50 (m, 1H), 7.54-7.65 (m, 5H), 7.80-7.84 (m, 2H), 7.98-8.04 (m, 2H), 8.39 (t, 1H, J = 5.5 Hz), 9.05 (s, 1H); m/z (APCI pos) 568.1 (100%) [M + H]. |
| E188 | 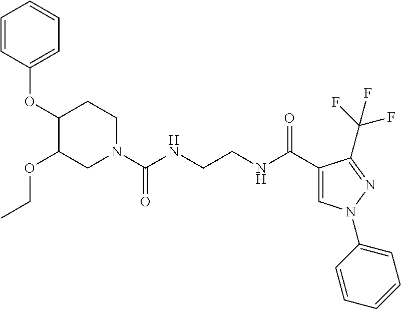 | 3-ethoxy-4- phenoxy-N-(2- (1-phenyl-3- (trifluoromethyl)- 1H- pyrazole-4- carboxamido)ethyl) piperidine- 1- carboxamide | Solid (45%); ¹H NMR (400 MHz, CDCl₃) δ 1.11 (t, 3H, J = 7.0 Hz), 1.72 (m, 1H), 2.06 (m, 1H), 3.33-3.70 (m, 11H), 4.38 (m, 1H), 5.29 (m, 1H), 6.94 (m, 3H), 7.28 (m, 2H), 7.33 (m, 1H), 7.43 (m, 1H), 7.50 (m, 2H), 7.71 (m, 2H), 8.44 (s, 1H); m/z (APCI pos) 546.3 (100%) [M + H]. |
| E189 | 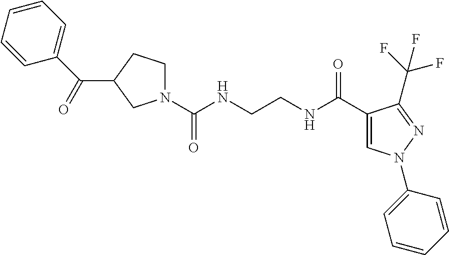 | N-(2-(3- benzoylpyrrolidine- 1- carboxamido)ethyl)- 1- phenyl-3- (trifluoromethyl)- 1H- pyrazole-4- carboxamide | ¹H NMR (400 MHz, CDCl₃) δ 2.25-2.30 (m, 2H), 3.42-3.58 (m, 6H), 3.70 (d, 2H, J = 6.3 Hz), 4.00-4.08 (m, 1H), 4.88 (t, 1H, J = 5.4 Hz), 7.36-7.41 (m, 2H), 7.48 (t, 4H, J = 7.7 Hz), 7.59 (t, 1H, J = 7.3 Hz), 7.71 (t, 2H, J = 8.0 Hz), 7.94 (d, 2H, J = 8.6 Hz), 8.46 (s, 1H); m/z (APCI pos) 500.1 (100%) [M + H]. |
| E190 | 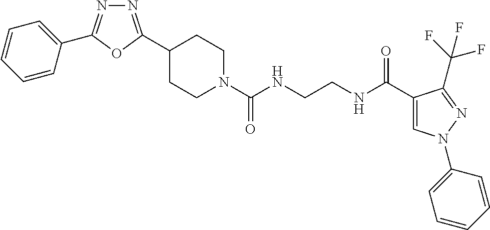 | 4-(5-phenyl- 1,3,4- oxadiazol-2- yl)-N-(2-(1- phenyl-3- (trifluoromethyl)- 1H- pyrazole-4- carboxamido)ethyl) piperidine- 1- carobxamide | White solid (33%); ¹H NMR (400 MHz, DMSO-d₆) δ 1.60-1.72 (m, 2H), 2.00-2.07 (m, 2H), 2.90-3.01 (m, 2H), 3.16-3.35 (m, 5H), 3.92-3.99 (m, 2H), 6.72 (t, 1H, J = 5.5 Hz), 7.45-7.50 (m, 1H), 7.57-7.63 (m, 5H), 7.79-7.83 (m, 2H), 7.97-8.01 (m, 2H), 8.40 (t, 1H, J = 5.6 Hz), 9.05 (s, 1H); m/z (APCI pos) 554.1 (100%) [M + H]. |
| E193 | 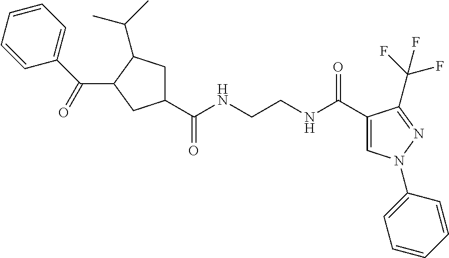 | N-(2-(3- benzoyl-4- isopropyl- pyrrolidine-1- carboxamido)ethyl)- 1- phenyl-3- (trifluouromethyl)- 1H- pyrazole-4- carboxamide | ¹H (400 MHz, CDCl₃) δ 0.85 (d, 3H, J = 6.6 Hz), 0.92 (d, 3H, J = 6.6 Hz), 1.61-1.70 (m, 1H), 2.61-2.70 (m, 1H), 3.20 (t, 1H, J = 8.9 Hz), 3.41 (t, 1H, J = 8.1 Hz), 3.47-3.56 (m, 4H), 3.64 (t, 1H, J = 8.9 Hz), 3.81-3.90 (m, 2H), 4.85-4.87 (t, 1H, J = 5.4 Hz), 7.35- 7.40 (m, 2H), 7.45- 7.50 (m, 2H), 7.60 (t, 1H, J = 6.7 Hz), 7.70 (d, 2H, J = 7.4 Hz), 7.95 (d, 2H, J = 7.2 Hz), 8.45 (s, 1H); m/z (APCI pos) (100%) 542.1 [M + H] |
| E199 | 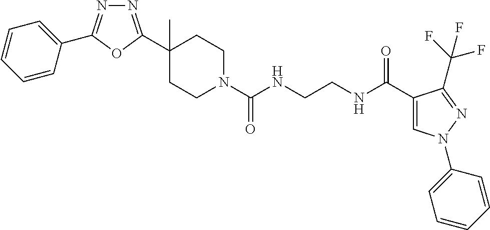 | 4-methyl-4- (5-phenyl- 1,3,4- oxaidazol-2- yl)-N-(2-(1- phenyl-3- (trifluoromethyl)- 1H- pyrazole-4- carboxamido)ethyl) piperidine- 1- carboxamide | White Solid (33%); ¹H NMR (400 MHz, DMSO-d₆) δ 1.38 (s, 3H), 1.57- 1.66 (m, 2H), 2.08-2.16 (m, 2H), 3.05-3.13 (m, 2H), 3.16-3.23 (m, 2H), 3.27-3.33 (m, 2H), 3.63-3.70 (m, 2H), 6.69 (t, 1H, J = 5.5 Hz), 7.45-7.50 (m, 1H), 7.57-7.64 (m, 5H), 7.79-7.83 (m, 2H), 7.99-8.03 (m, 2H), 8.39 (t, 1H, J = 5.5 Hz), 9.05 (s, 1H); m/z (APCI pos) (100%) 568.1 [M + H]. |
| E201 | 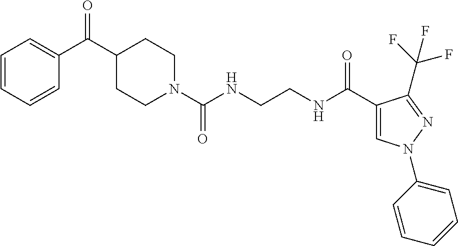 | 4-benzoyl-N- (2-(1-phenyl- 3- (trifluoromethyl)- 1H- pyrazole-4- carboxamido)ethyl) piperidine- 1- carboxamide | Solid (57%); ¹H NMR (400 MHz, DMSO-d₆) δ 1.36-1.48 (m, 2H), 1.70-1.78 (m, 2H), 2.82-2.92 (m, 2H), 3.16-3.22 (m, 2H), 3.26-3.32 (m, 2H), 3.57-3.66 (m, 1H), 3.95-4.03 (m, 2H), 6.66 (t, 1H, J = 5.5 Hz), 7.44-7.68 (m, 6H), 7.80-7.84 (m, 2H), 7.97-8.01 (m, 2H), 8.40 (t, 1H, J = 5.5 Hz), 9.05 (s, 1H); m/z 514.1 (APCI pos) (100%) [M + H]. |
| E205 | 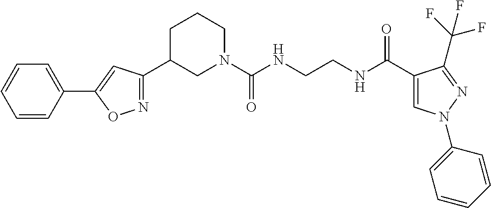 | N-(2-(1- phenyl-3- (trifluoromethyl)- 1H- pyrazole-4- carboxamido)ethyl)- 3-(5- phenylisoxazol- 3- yl)piperidine- 1- carboxamide | Solid (71%); ¹H NMR (400 MHz, DMSO-d₆) δ 1.40-1.72 (m, 3H), 2.03-2.09 (m, 1H), 2.72-2.93 (m, 3H), 3.17-3.35 (m, 4H), 3.92-3.99 (m, 1H), 4.16-4.22 (m, 1H), 6.75 (t, 1H, J = 5.5 Hz), 6.99 (s, 1H), 7.43-7.62 (m, 6H), 7.78-7.83 (m, 4H), 8.40 (t, 1H, J = 5.5 Hz), 9.06 (s, 1H); m/z 553.0 (APCI pos) (50%) [M + H]. |
| E206 | 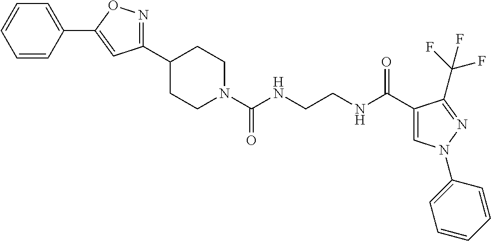 | N-(2-(1- phenyl-3- (trifluoromethyl)- 1H- pyrazole-4- carboxamido)ethyl)- 4-(5- phenylisoxazol- 3- yl)piperidine- 1- carboxamide | Solid (80%); ¹H NMR (400 MHz, DMSO-d₆) δ 1.49-1.60 (m, 2H), 1.85-1.92 (m, 2H), 2.81-2.90 (m, 2H), 2.91-3.00 (m, 1H), 3.17-3.24 (m, 2H), 3.28-3.35 (m, 2H), 3.99-4.05 (m, 2H), 6.70 (t, 1H, J = 5.5 Hz), 7.01 (s, 1H), 7.45-7.64 (m, 6H), 7.79-7.84 (m, 4H), 8.41 (t, 1H, J = 5.5 Hz), 9.06 (s, 1H); ); m/z 553.1 (APCI pos) (50%) [M + H]. |
| E207 | 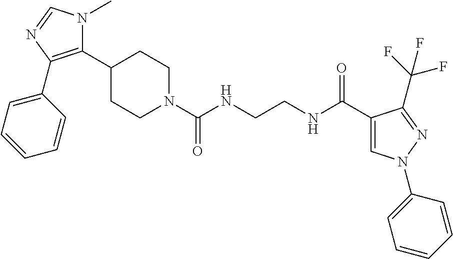 | 4-(1-methyl- 4-phenyl-1H- imidazol-5- yl)-N-(2-(1- phenyl-3- (trifluoromethyl)- 1H- pyrazole-4- carboxamido)ethyl) piperidine- 1- carboxamide | Solid (46%); ¹H NMR (400 MHz, DMSO-d₆) δ 1.60-1.80 (m, 4H), 2.64-2.73 (m, 2H), 2.99-3.09 (m, 1H), 3.16-3.33 (m, 4H), 3.64 (s, 3H), 4.00- 4.08 (m, 2H), 6.66 (t, 1H, J = 5.5 Hz), 7.22- 7.28 (m, 1H), 7.33- 7.42 (m, 4H), 7.45- 7.50 (m, 1H), 7.53 (s, 1H), 7.58-7.63 (m, 2H), 7.80-7.83 (m, 2H), 8.40 (t, 1H, J = 5.5 Hz), 9.06 (s, 1H); m/z 566.3 (APCI pos) (50%) [M + H]. |
| E208 | 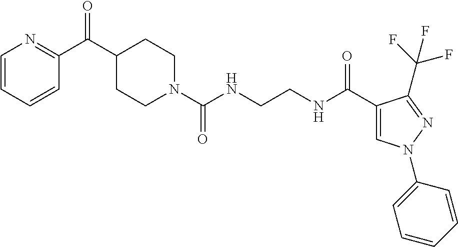 | N-(2-(1- phenyl-3- (trifluoromethyl)- 1H- pyrazole-4- carboxamido) ethyl)-4- picolinoyl- piperidine-1- carboxamide | Solid (41%); ¹H NMR (400 MHz, CDCl₃) δ 1.66- 1.70 (m, 2H), 1.93- 1.96 (m, 2H), 3.03 (t, J = 13.6 Hz, 2H), 3.50- 3.58 (m, 4H), 4.00- 4.06 (m, 3H), 5.33 (br, 1H), 7.39 (t, J = 7.2 Hz, 2H), 7.46- 7.51 (m, 3H), 7.72 (d, J = 7.6 Hz, 2H), 7.85 (t, J = 7.6 Hz, 1H), 8.01 (d, J = 8.0 Hz, 1H), 8.48 (s, 1H), 8.66-8.88 (m, 1H); m/z (APCI pos) 515.1 [M + H]. |
| E209 | 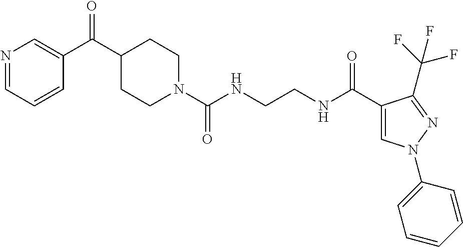 | 4-nicotinoyl- N-(2-(1- phenyl-3- (trifluoromethyl)- 1H- pyrazole-4- carboxamido)ethyl) piperidine- 1- carboxamide | ¹H NMR (400 MHz, CDCl₃) δ 1.72-1.78 (m, 2H), 1.89-1.93 (m, 2H), 3.01 (t, J = 14.4 Hz, 2H), 3.38-3.43 (m, 1H), 3.48-3.50 (m, 2H), 3.55-3.59 (m, 2H), 4.03 (d, J = 13.2 Hz, 2H), 5.42 (t, J = 4.8 Hz, 1H), 7.25 (br, 1H), 7.38-7.52 (m, 4H), 7.71 (d, J = 7.2 Hz, 2H), 8.21 (d, J = 8.0 Hz, 1H), 8.47 (s, 1H), 8.80 (d, J = 3.6 Hz, 1H), 9.15 (s, 1H); m/z (APCI pos) 515.1 [M + H]. |
| E210 | 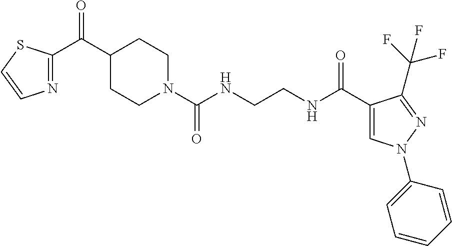 | N-(2-(1- phenyl-3- (trifluoromethyl)- 1H- pyrazole-4- carboxamido)ethyl)- 4- (thiazole-2- carbonyl) piperidine-1- carboxamide | ¹H NMR (400 MHz, CDCl₃) δ 1.70-1.80 (m, 2H), 1.99-2.04 (m, 2H), 3.01 (t, J = 14.4 Hz, 2H), 3.49-3.58 (m, 4H), 3.75-3.79 (m, 1H), 4.02-4.06 (m, 2H), 5.38 (br, 1H), 7.31 (br, 1H), 7.40 (t, J = 7.6 Hz, 1H), 7.50 (t, J = 8.4 Hz, 2H), 7.69 (d, J = 3.2 Hz, 1H), 7.72 (d, J = 7.6 Hz, 2H), 8.01 (d, J = 3.2 Hz, 1H), 8.48 (s, 1H). m/z (APCI pos) 521.0 (M + H). |
| E211 | 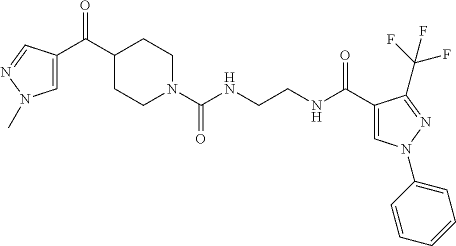 | 4-(1-methyl- 1H-pyrazole- 4-carbonyl)- N-(2-(1- phenyl-3- (trifluoromethyl)- 1H- pyrazole-4- carboxamido)ethyl) piperidine- 1- carboxamide | ¹H NMR (400 MHz, CDCl₃) δ 1.67-1.84 (m, 4H), 2.92-3.01 (m, 3H), 3.49 (br, 2H), 3.57 (br, 2H), 3.94 (s, 3H), 3.98 (br, 1H), 4.02 (br, 1H), 5.38 (br, 1H), 7.30 (br, 1H), 7.40 (t, J = 6.8 Hz, 1H), 7.50 (t, J = 8.4 Hz, 2H), 7.72 (d, J = 8.0 Hz, 2H), 7.87 (d, J = 4.8 Hz, 2H), 8.48 (s, 1H); m/z (APCI pos) 518.1 [M + H]. |
| E212 | 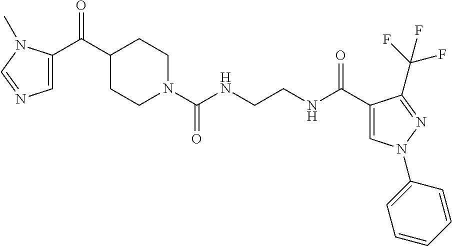 | 4-(1-methyl- 1H-imidazole- 5-carbonyl)- N-(2-(1- phenyl-3- (trifluoromethyl)- 1H- pyrazole-4- carboxamido)ethyl) piperidine- 1- carboxamide | ¹H NMR (400 MHz, CDCl₃) δ 1.64-1.72 (m, 2H), 1.93 (d, J = 13.6 Hz, 2H), 2.98 (t, J = 12.8 Hz, 2H), 3.48-3.57 (m, 4H), 3.86 (tt, J = 3.6, 12.0 Hz, 1H), 3.98 (s, 3H), 4.01 (br, 1H), 4.06 (br, 1H), 5.28 (br, 1H), 7.05 (s, 1H), 7.16 (s, 1H), 7.37 (br, 1H), 7.39 (t, J = 7.2 Hz, 1H), 7.50 (t, J = 8.0 Hz, 2H), 7.72 (d, J = 8.4 Hz, 2H), 8.46 (s, 1H); m/z (APCI pos) 518.1 [M + H]. |
| E225 | 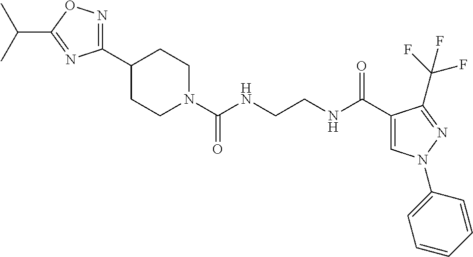 | 4-(5- isopropyl- 1,2,4- oxadiazol-3- yl)-N-(2-(1- phenyl-3- (trifluoromethyl)- 1H- pyrazole-4- carboxamido)ethyl) piperidine- 1- carboxamide | White solid (69%); ¹H NMR (400 MHz, DMSO-d₆) δ 1.29 (d, 6H, J = 7.0 Hz), 1.45-1.58 (m, 2H), 1.84-1.91 (m, 2H), 2.81-2.90 (m, 2H), 2.92-3.01 (m, 1H), 3.16-3.38 (m, 5H), 3.91-3.99 (m, 2H), 6.66 (t, 1H, J = 5.5 Hz), 7.45-7.50 (m, 1H), 7.58-7.64 (m, 2H), 7.79-7.83 (m, 2H), 8.40 (t, 1H, J = 5.5 Hz), 9.05 (s, 1H); m/z (APCI pos) 520.1 (100%). |
| E226 | 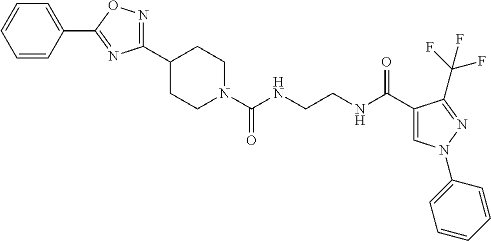 | 4-(5-phenyl- 1,2,4- oxadiazol-3- yl)-N-(2-(1- phenyl-3- (trifluoromethyl)- 1H- pyrazole-4- carboxamido)ethyl) piperidine- 1- carboxamide | White solid (79%); ¹H NMR (400 MHz, DMSO-d₆) δ 1.56-1.67 (m, 2H), 1.92-1.99 (m, 2H), 2.87-2.95 (m, 2H), 3.05-3.13 (m, 1H), 3.18-3.24 (m, 2H), 3.28-3.34 (m, 2H), 3.95-4.03 (m, 2H), 6.70 (t, 1H, J = 5.5 Hz), 7.44-7.49 (m, 1H), 7.57-7.65 (m, 4H), 7.68-7.73 (m, 1H), 7.79-7.83 (m, 2H), 8.07-8.10 (m, 2H), 8.40 (t, 1H, J = 5.5 Hz), 9.05 (s, 1H); m/z (APCI pos) (100%) 554.1 [M + H]. |
| E227 | 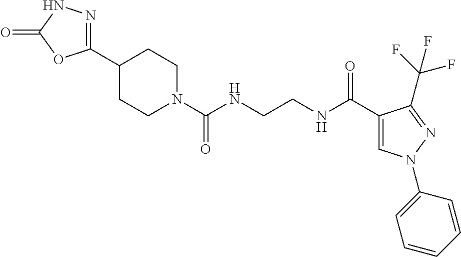 | 4-(5-oxo-4,5- dihydro- 1,3,4- oxadiazol-2- yl)-N-(2-(1- phenyl-3- (trifluoromethyl)- 1H- pyrazole-4- carboxamido)ethyl) piperidine- 1- carboxamide | White solid (61%); ¹H NMR (400 MHz, DMSO-d₆) δ 1.40-1.52 (m, 2H), 1.80-1.88 (m, 2H), 2.80-2.89 (m, 3H), 3.15-3.22 (m, 2H), 3.26-3.32 (m, 2H), 3.96-3.93 (m, 2H), 6.68 (t, 1H, J = 5.5 Hz), 7.45-7.50 (m, 1H), 7.58-7.64 (m, 2H), 7.78-7.83 (m, 2H), 8.40 (t, 1H), J = 5.5 Hz), 9.05 (s, 1H), 12.09 (s, 1H); m/z (APCI pos) (100%) 494.1 [M + H]. |

### Example E91

### N-(2-(trans-4-Benzamidocyclohexanecarboxamido) ethyl)-1-phenyl-3-(trifluoromethyl)-1H-pyrazole-4-carboxamide

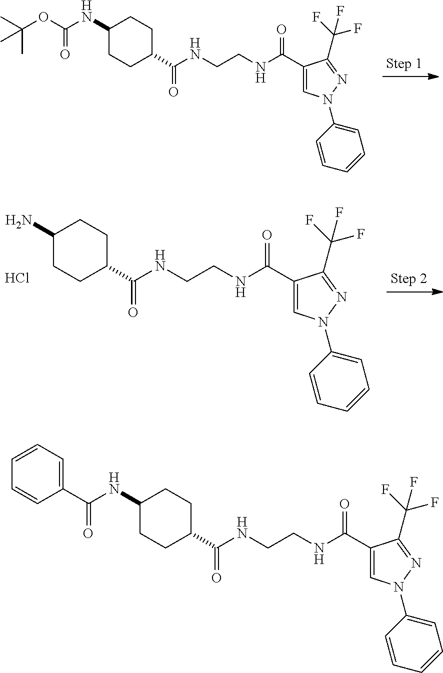

### Step 1

Following the procedure described in example E5, N-(2-(trans-4-aminocyclohexanecarboxamido)ethyl)-1-phenyl-3-(trifluoromethyl)-1H-pyrazole-4-carboxamide hydrochloride was obtained as a white solid (98%) from tert-butyl trans-4-(2-(1-phenyl-3-(trifluoromethyl)-1H-pyrazole-4-carboxamido)ethylcarbamoyl)cyclohexylcarbamate: 1H NMR (400 MHz, DMSO-d₆) δ 1.22-1.46 (m, 4H), 1.75 (m, 2H), 1.92 (m, 2H), 2.03-2.11 (m, 1H), 2.90-3.00 (m, 1H), 3.18-3.31 (m, 4H), 4.76 (br, 2H), 7.45-7.50 (m, 1H), 7.58-7.64 (m, 2H), 7.81-7.86 (m, 2H), 8.04 (t, 1H, J=5.5 Hz), 8.56 (t, 1H, J=5.5 Hz), 9.30 (s, 1H); m/z (APCI pos) 424.0 (100%) [M+H].

### Step 2

To N-(2-(trans-4-aminocyclohexanecarboxamido)ethyl)-1-phenyl-3-(trifluoromethyl)-1H-pyrazole-4-carboxamide hydrochloride (100 mg, 0.22 mmol) in DCM (10 mL) were successively added benzoic acid (27 mg, 0.22 mmol), EDAC.HCl (54 mg, 0.28 mmol), HOBt.H₂O (41 mg, 0.30 mmol) and triethylamine (44 mg, 0.43 mmol). The mixture was stirred at room temperature for 18 hours then DCM was added (100 mL). The organic layer was successively washed with 2N HCl (50 mL), 10% potassium carbonate aqueous solution (50 mL) and brine (2×50 mL). The organic layer was dried over MgSO₄ then concentrated. The residue was purified by MPLC (DCM/MeOH 95/5->90/10) to yield N-(2-(trans-4-benzamidocyclohexanecarboxamido)ethyl)-1-phenyl-3-(trifluoromethyl)-1H-pyrazole-4-carboxamide (76 mg, 67%): ¹H NMR (400 MHz, DMSO-d₆) δ 1.28-1.50 (m, 4H), 1.76-1.90 (m, 4H), 2.02-2.10 (m, 1H), 3.16-3.34 (m, 4H), 3.67-3.78 (m, 1H), 7.40-7.53 (m, 4H), 7.58-7.64 (m, 2H), 7.80-7.88 (m, 5H), 8.09 (d, 1H, J=7.9 Hz), 8.37 (t, 1H, J=5.5 Hz), 9.02 (s, 1H); m/z (APCI pos) 528.1 (100%) [M+H].

The compound of the following structure was prepared from the corresponding amine hydrochloride, using a similar method to that described above.

| Ex # | Structure | Name | Physical Data |
|---|---|---|---|
| E92 | 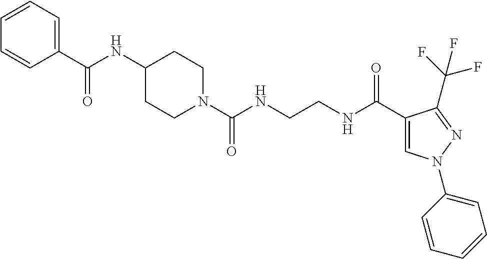 | 4-benzamido- N-(2-(1- phenyl-3- (trifluoromethyl)- 1H- pyrazole-4- carboxamido) ethyl)piperidine- 1- carboxamide | White solid (40%); ¹H NMR (400 MHz, DMSO-d₆) δ 1.35-1.46 (m, 2H), 1.72-1.79 (m, 2H), 2.74-2.83 (m, 2H), 3.15-3.34 (m, 5H), 3.90-4.00 (m, 2H), 6.70 (t, 1H, J = 5.3 Hz), 7.42-7.54 (m, 4H), 7.57-7.63 (m, 2H), 7.80-7.86 (m, 4H), 8.24 (d, 1H, J = 7.8 Hz), 8.42 (t, 1H, J = 5.5 Hz), 9.06 (s, 1H); m/z (APCI pos) 529.0 (100%) [M + H]. |

### Example E110

### N-(2-(trans-4-benzoyl-N-methylcyclohexanecarboxamido)ethyl)-1-phenyl-3-(trifluoromethyl)-1H-pyrazole-4-carboxamide

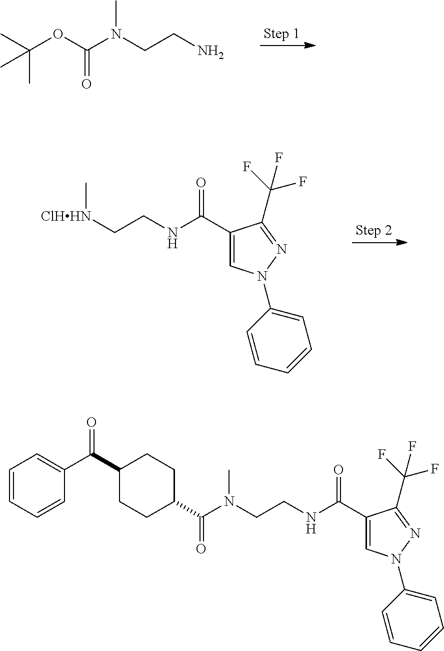

### Step 1

To 1-phenyl-3-(trifluoromethyl)-1H-pyrazole-4-carboxylic acid (500 mg, 1.95 mmol) in DCM (70 mL) were successively added tert-butyl 2-aminoethyl(methyl)carbamate (340 mg, 1.95 mmol), HOBt.H₂O (369 mg, 2.73 mmol), EDAC.HCl (486 mg, 2.54 mmol) and triethylamine (395 mg, 3.90 mmol). The mixture was stirred at room temperature for 18 hours then diluted with DCM (70 mL). The organic layer was successively washed with 2N HCl, 10% aqueous potassium carbonate and brine. The organic layer was dried over MgSO₄ then concentrated to yield a white solid (671 mg, 84%). To this solid (650 mg, 1.58 mmol) was added 2N HCl in dioxane (10 mL) and the mixture was stirred for 10 minutes. Concentration yielded N-(2-(methylamino)ethyl)-1-phenyl-3-(trifluoromethyl)-1H-pyrazole-4-carboxamide hydrochloride as a white solid (550 mg, quant.): ¹H NMR (400 MHz, DMSO-d₆) δ 2.60 (s, 3H), 3.40-3.68 (m, 4H), 7.45-7.50 (m, 1H), 7.58-7.64 (m, 2H), 7.82-7.86 (m, 2H), 8.94 (t, 1H, J=5.5 Hz), 9.00 (br, 2H), 9.53 (s, 1H).

### Step 2

Following the procedure described above for the amide formation, N-(2-(trans-4-benzoyl-N-methylcyclohexanecarboxamido)ethyl)-1-phenyl-3-(trifluoromethyl)-1H-pyrazole-4-carboxamide was obtained as a white solid (32%) from N-(2-(methylamino)ethyl)-1-phenyl-3-(trifluoromethyl)-1H-pyrazole-4-carboxamide hydrochloride and trans-4-benzoylcyclohexanecarboxylic acid: mixture of cis and trans amides: ¹H NMR (400 MHz, DMSO-d₆) δ 1.20-1.90 (m, 8H), 2.55-2.64 (m, 1H), 2.86 (s, 1.4H), 3.07 (s, 1.6H), 3.32-3.54 (m, 5H), 7.44-7.66 (m, 6H), 7.76-7.83 (m, 2H), 7.93-7.99 (m, 2H), 8.38 (t, 0.6H, J=5.5 Hz), 8.53 (t, 0.4H, J=5.5 Hz), 8.99 (s, 0.6H), 9.05 (s, 0.4H); m/z (APCI pos) 527.2 (100%) [M+H].

### Example E111

### Ethyl 1-(4-(1-phenyl-3-(trifluoromethyl)-1H-pyrazole-4-carboxamido)butanoyl)piperidine-4-carboxylate

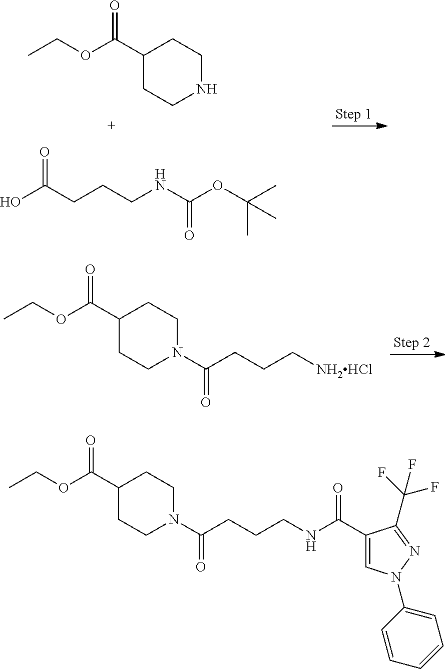

Step 1

To ethyl piperidine-4-carboxylate (500 mg, 3.18 mmol) in DCM (70 mL) were successively added 4-(tert-butoxycarbonylamino)butanoic acid (646 mg, 3.18 mmol), HOBt.H₂O (602 mg, 4.45 mmol), EDAC.HCl (793 mg, 4.13 mmol) and triethylamine (644 mg, 6.36 mmol). The mixture was stirred at room temperature for 18 hours then diluted with DCM (70 mL). The organic layer was successively washed with 2N HCl, 10% aqueous potassium carbonate and brine. The organic layer was dried over MgSO₄ then concentrated to yield a clear paste (792 mg, 73%). To this paste (770 mg, 2.25 mmol) was added 2N HCl in dioxane (10 mL) and the mixture was stirred for 10 minutes. Concentration yielded ethyl 1-(4-aminobutanoyl)piperidine-4-carboxylate hydrochloride as a white solid (620 mg, quant.): ¹H NMR (400 MHz, DMSO-d₆) δ 1.18 (t, 3H, J=7.0 Hz), 1.75-1.85 (m, 4H), 2.35-2.81 (m, 7H), 3.00-3.11 (m, 1H), 3.65-3.85 (m, 2H), 4.07 (q, 2H, J=7.0 Hz), 4.19-4.26 (m, 1H).

The compound of the following structure was prepared from the corresponding amine, using a similar method to that described above.

| Structure | Name | Physical Data |
|---|---|---|
| 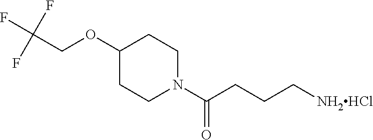 | 4-amino-1- (4-(2,2,2- trifluoro- ethoxy) piperidin-1- yl)butan-1- one hydrochloride | Clear paste (quant.); ¹H NMR (400 MHz, DMSO-d₆) δ 1.30-1.50 (m, 2H), 1.70- 1.90 (m, 3H), 2.40-2.45 (m, 1H), 2.75-2.81 (m, 1H), 3.00-3.22 (m, 2H), 3.44- 3.52 (m, 2H), 3.60-3.90 (m, 4H), 4.05-4.20 (m 2H). |

### Step 2

Following the procedure described above for the amide formation, ethyl 1-(4-(1-phenyl-3-(trifluoromethyl)-1H-pyrazole-4-carboxamido)butanoyl)piperidine-4-carboxylate was obtained as a clear solid (56%) from ethyl 1-(4-aminobutanoyl)piperidine-4-carboxylate hydrochloride and 1-phenyl-3-(trifluoromethyl)-1H-pyrazole-4-carboxylic acid: ¹H NMR (400 MHz, DMSO-d₆) δ 1.17 (t, 3H, J=7.1 Hz), 1.30-1.54 (m, 2H), 1.70-1.88 (m, 4H), 2.34-2.41 (m, 2H), 2.53-2.62 (m, 1H), 2.64-2.74 (m, 1H), 3.03-3.12 (m, 1H), 3.21-3.28 (m, 2H), 3.75-3.83 (m, 1H), 4.06 (q, 2H, J=7.1 Hz), 4.20-4.27 (m, 1H), 7.44-7.50 (m, 1H), 7.57-7.63 (m, 2H), 7.80-7.85 (m, 2H), 8.32 (t, 1H, J=5.5 Hz), 9.06 (s, 1H); m/z (APCI pos) 481.3 (100%) [M+H].

The compound of the following structure was prepared from the corresponding amine, using a similar method to that described above.

| Ex # | Structure | Name | Physical Data |
|---|---|---|---|
| E112 | 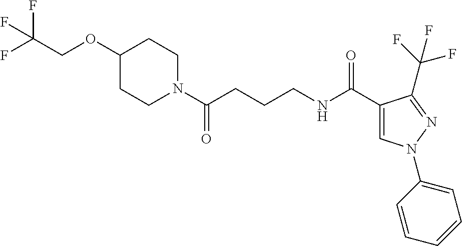 | N-(4-oxo-4- (4-(2,2,2- trifluoro- ethoxy) piperidin-1- yl)butyl)- 1-phenyl-3- (trifluoro- methyl)-1H- pyrazole-4- carboxamide | Clear paste (51%); ¹H NMR (400 MHz, DMSO-d₆) δ 1.30-1.50 (m, 2H), 1.70- 1.90 (m, 4H), 2.35-2.42 (m, 2H), 3.03-3.29 (m, 4H), 3.61-3.75 (m, 2H), 3.81-3.90 (m, 1H), 4.07 (q, 2H, J = 9.4 Hz), 7.44-7.50 (m, 1H), 7.57- 7.63 (m, 2H), 7.80-7.85 (m, 2H), 8.32 (t, 1H, J = 5.5 Hz), 9.06 (s, 1H); m/z (APCI pos) 507.3 (100%) [M + H]. |

### Example E114

### N-(2-(1-Phenyl-3-(trifluoromethyl)-1H-pyrazole-4-carboxamido)ethyl)-4-(phenylsulfonamido)piperidine-1-carboxamide

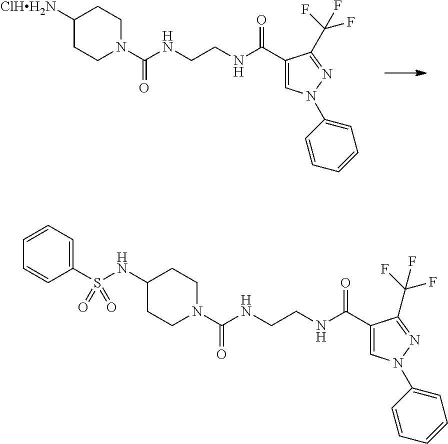

To 4-amino-N-(2-(1-phenyl-3-(trifluoromethyl)-1H-pyrazole-4-carboxamido)ethyl)piperidine-1-carboxamide hydrochloride (60 mg, 0.13 mmol) in THF (2 mL) at 0° C. were added triethylamine (26 mg, 0.26 mmol) and phenylsulfonyl chloride (23 mg, 0.13 mmol). The mixture was stirred at 0° C. for 3 hours then at room temperature for 18 hours. Concentration yielded a residue which was purified by MPLC (DCM/MeOH 95/5) to give N-(2-(1-phenyl-3-(trifluoromethyl)-1H-pyrazole-4-carboxamido)ethyl)-4-(phenylsulfonamido)piperidine-1-carboxamide. White solid (56 mg, 76%): ¹H NMR (400 MHz, d₆-DMSO) δ 1.12-1.30 (m, 4H), 1.45-1.53 (m, 2H), 2.66-2.75 (m, 2H), 3.10-3.28 (m, 4H), 3.67-3.75 (m, 2H), 6.58 (t, 1H, J=5.4 Hz), 7.44-7.50 (m, 1H), 7.57-7.63 (m, 4H), 7.73-7.85 (m, 5H), 8.32 (t, 1H, J=5.5 Hz), 9.03 (s, 1H); m/z (APCI pos) 565.2 (100%). [M+H].

### Example E122

### trans-N-1-Methyl-N4-(2-(1-phenyl-3-(trifluoromethyl)-1H-pyrazole-4-carboxamido)ethyl)-N-1-(pyridin-3-ylmethyl)cyclohexane-1,4-dicarboxamide

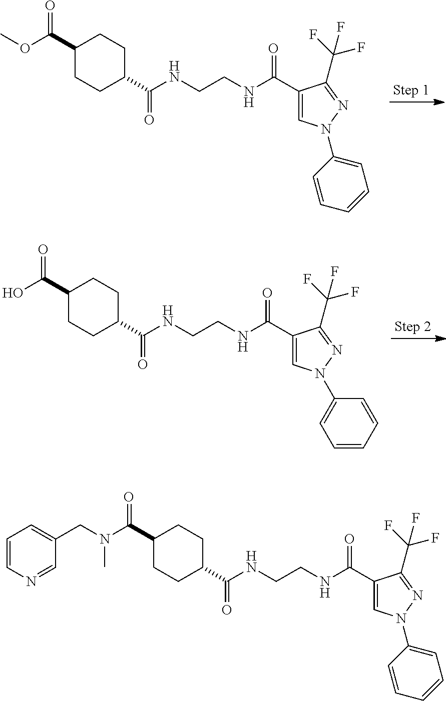

### Step 1

To a solution of trans-methyl 4-(2-(1-phenyl-3-(trifluoromethyl)-1H-pyrazole-4-carboxamido)ethylcarbamoyl)cyclohexanecarboxylate (3.94 g, 8.45 mmol) in THF/water (1:1, 50 mL) was added lithium hydroxide monohydrate (1.06 g, 25.3 mmol). The mixture was stirred for 16 hours, the water removed under vacuum, and the resulting mixture diluted with water and acidified with 4N HCl. The resulting suspension was filtered, the solid was dried under vacuum and triturated with 10% MeOH/ether to give trans-4-(2-(1-phenyl-3-(trifluoromethyl)-1H-pyrazole-4-carboxamido)ethylcarbamoyl)cyclohexanecarboxylic acid (3.77 g, 99% yield) as a solid: ¹H NMR (400 MHz, DMSO-d₆) δ 1.31 (m, 4H), 1.76 (m, 2H), 1.90 (m, 2H), 2.09 (m, 2H), 3.20 (m, 2H), 3.26 (m, 2H), 7.48 (m, 1H), 7.61 (m, 2H), 7.82 (m, 3H), 8.37 (m, 1H), 9.05 (m, 1H), 12.03 (br, 1H); m/z (APCI neg) 451.2 (100%) [M−H].

### Step 2

### General Procedure for Preparation of Amide Library

To a Jones tube was added PS-carbodiimide (0.384 g, 0.510 mmol) (pre-swelled in 2 mL of THF), followed by HOBt.H₂O (0.005 g, 0.0370 mmol) and an amine (0.170 mmol) in THF (100 μl). trans-4-(2-(1-Phenyl-3-(trifluoromethyl)-1H-pyrazole-4-carboxamido)ethylcarbamoyl)cyclohexanecarboxylic acid (0.100 g, 0.221 mmol) was then added as a solution in 300 μl of 1:1 THF/DMF. For the cases where the amine was a hydrochloride, DIPEA (0.0592 mL, 0.340 mmol) and MP-carbonate (0.108 g, 0.340 mmol) were also added to the tube. The tube was placed on an orbital shaker and agitated at 60 rpm for 16 hours. Each tube was then treated with PS-trisamine (0.00910 g, 0.0374 mmol) and PS-isocyanate (0.0950 g, 0.170 mmol) and agitated further for 4 hours. The tubes were then drained, the resin washed with THF repeatedly, and the solvent removed using a Genevac vacuum centrifuge. All compounds were purified, if necessary, using silica gel chromatography to >95% purity.

Compounds of the following structures were prepared from the corresponding primary or secondary amines, using a similar method to that described above.

| Ex # | Structure | Name | Physical Data |
|---|---|---|---|
| E122 | 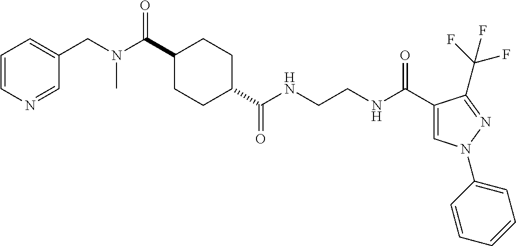 | trans-N1- methyl-N4-(2- (1-phenyl-3- (trifluoromethyl)- 1H- pyrazole-4- carboxamido)- ethyl)-N1- (pyridin-3- ylmethyl)cyclo- hexane-1,4- dicarboxamide | Solid (30%); ¹H NMR (400 MHz, DMSO-d₆) δ 1.41 (m, 4H), 1.72 (m, 4H), 2.10 (m, 1H), 2.63 (m, 1H), 2.80 (s, 0.8H), 2.99 (s, 2.2H), 3.24 (m, 4H), 4.51 (s, 1.5H), 4.67 (s, 0.5H), 7.37 (m, 1H), 7.48 (m, 1H), 7.59 (m, 3H), 7.82 (m, 3H), 8.37 (m, 1H), 8.47 (m, 2H), 9.03 (m, 1H); m/z 557.2 (APCI pos) (100%) [M + H]. |
| E115 | 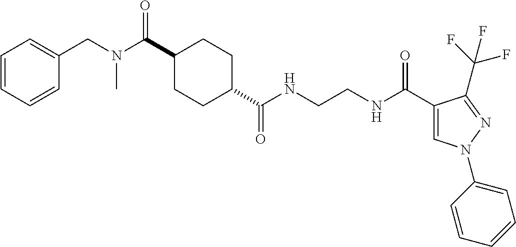 | trans-N1- benzyl-N1- methyl-N4- (2-(1- phenyl-3- (trifluoromethyl)- 1H- pyrazole-4- carboxamido) ethyl)cyclo- hexane-1,4- dicarboxamide | Solid (25%); ¹H NMR (400 MHz, DMSO-d₆) δ 1.41 (m, 4H), 1.71 (m, 4H), 2.07 (m, 1H), 2.63 (m, 1H), 2.79 (s, 1H (rotamer)), 2.94 (s, 2H (rotamer)), 3.23 (m, 4H), 4.48 (m, 1.3H (rotamer)), 4.61 (m, 0.7 H (rotamer)), 7.17 (m, 2H), 7.22- 7.40 (m, 3H), 7.48 (m, 1H), 7.61 (m, 2H), 7.82 (m, 3H), 8.36 (m, 1H), 9.03 (m, 1H); m/z (APCI pos) 556.1 (100%) [M + H]. |
| E116 | 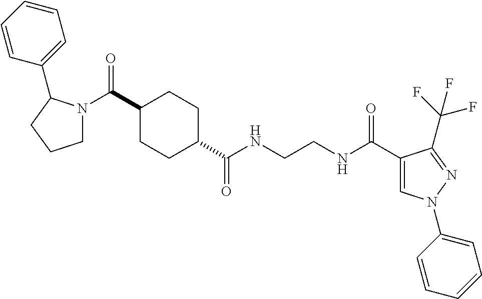 | 1-phenyl-N- (2-(trans-4- (2- phenyl- pyrrolidine-1- carbonyl)cyclo- hexanecar- boxamido)ethyl)- 3- (trifluoromethyl)- 1H- pyrazole-4- carboxamide | Solid (9%); ¹H NMR (400 MHz, DMSO-d₆) δ 0.84 (m, 1H), 1.35 (m, 3H), 1.37-2.33 (m, 8H), 3.24 (6H), 3.58 (m, 2H), 5.05 (m, 1H), 7.09-7.36 (m, 5H), 7.48 (m, 1H), 7.61 (m, 2H), 7.80 (m, 3H), 8.33 (m, 1H), 9.02 (d, 1H, rotamers); m/z (APCI pos) 582.1 (100%) [M + H]. |
| E118 | 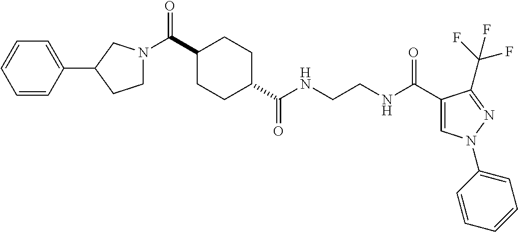 | 1-phenyl-N- (2-(trans-4- (3- phenyl- pyrrolidine-1- carbonyl)cyclo- hexanecar- boxamido)ethyl)- 3- (trifluoromethyl)- 1H- pyrazole-4- carboxamide | Solid (1%); ¹H NMR (400 MHz, DMSO-d₆) δ 1.37 (m, 4H), 1.75 (m, 4H), 1.90 (m, 0.5H), 2.08 (m, 1H), 2.19 (m, 0.5H), 2.28 (m, 0.5H), 2.41 (m, 1H), 3.19 (m, 2H), 3.27 (m, 2H), 3.39 (m, 1H), 3.55 (m, 0.5H), 3.71 (m, 1H), 3.79 (m, 1H), 3.97 (m, 1H), 7.28 (m, 5H), 7.48 (m, 1H), 7.60 (m, 2H), 7.81 (m, 3H), 8.35 (m, 1H), 9.03 (m, 1H); m/z 582.2 (APCI pos) (100%) [M + H]. |
| E123 | 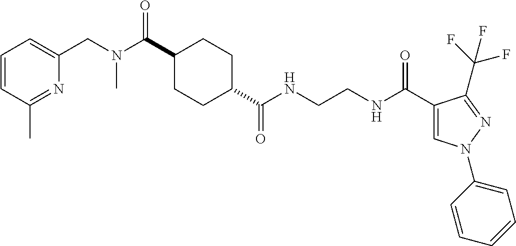 | trans-N1- methyl-N1- ((6- methylpyridin- 2- yl)methyl)- N4-(2-(1- phenyl-3- (trifluoromethyl)- 1H- pyrazole-4- carboxamido) ethyl)cyclo- hexane-1,4- dicarboxamide | Solid (37%); ¹H NMR (400 MHz, CD₃OD) δ 1.42 (m, 4H), 1.83 (m, 4H), 2.21 (m, 1H), 2.49 (m, 3H), 2.71 (m, 1H), 3.12 (m, 2H), 3.43 (m, 4H), 4.63 (s, 2H), 4.67 (s, 1H), 7.00 (m, 1H), 7.16 (m, 1H), 7.44 (m, 1H), 7.56 (m, 2H), 7.66 (m, 1H), 7.81 (m, 2H), 8.71 (m, 1H); m/z 571.2 (APCI pos) (100%) [M + H]. |
| E124 | 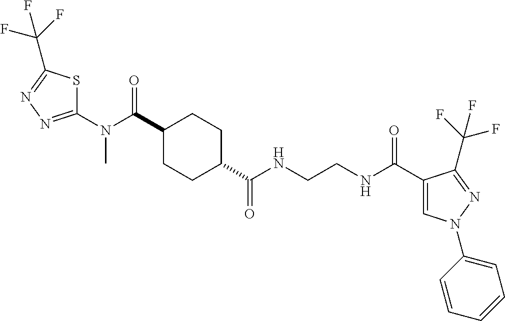 | trans-N1- methyl-N4- (2-(1- phenyl-3- (trifluoromethyl)- 1H- pyrazole-4- carboxamido) ethyl)-N1- (5- (trifluoromethyl)- 1,3,4- thiadiazol- 2- yl)cyclohexane- 1,4- dicarboxamide | Solid (6%); ¹H NMR (400 MHz, CD₃OD) δ 1.51 (m, 4H), 1.96 (m, 4H), 2.26 (m, 1H), 3.04 (m, 1H), 3.44 (m, 4H), 3.87 (s, 3H), 7.45 (m, 1H), 7.57 (m, 2H), 7.83 (m, 2H), 8.72 (m, 1H); m/z 618.0 (APCI pos) (100%) [M + H]. |
| E125 | 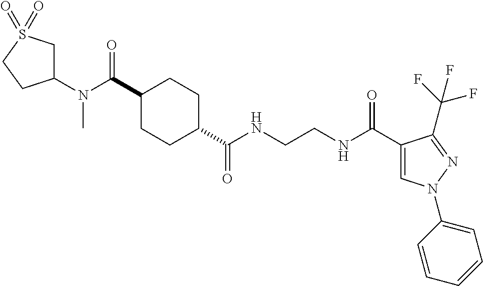 | trans-N1- (1,1- dioxidotetra hydro- thiophen-3- yl)-N1- methyl-N4- (2-(1- phenyl-3- (trifluoromethyl)- 1H- pyrazole-4- carboxamido) ethyl)- cyclohexane- 1,4- dicarboxamide | Solid (32%); ¹H NMR (400 MHz, DMSO-d₆) δ 1.38 (m, 4H), 1.73 (m, 4H), 2.13 (m, 3H), 2.69 (s, 2H), 2.93 (s, 2H), 3.09 (m, 2H), 3.23 (m, 7H), 7.48 (m, 1H), 7.61 (m, 2H), 7.82 (m, 3H), 8.36 (m, 1H), 9.04 (s, 1H); m/z 584.0 (APCI pos) (100%) [M + H]. |
| E126 | 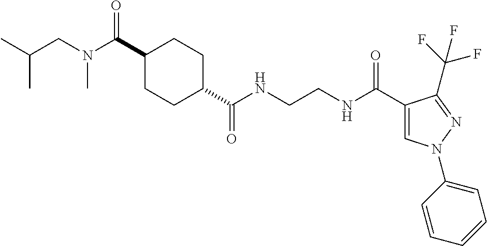 | trans-N1- isobutyl-N1- methyl-N4- (2-(1- phenyl-3- (trifluoromethyl)- 1H- pyrazole-4- carboxamido) ethyl)cyclo- hexane-1,4- dicarboxamide | Solid (31%); ¹H NMR (400 MHz, CD₃OD) δ 0.86 (d, 3H, J = 7.0 Hz), 0.92 (d, 3H, J = 6.6 Hz), 1.50 (m, 4H), 1.83 (m, 5H), 2.20 (m, 1H), 2.65 (m, 1H), 2.88 (s, 1H), 3.07 (s, 2H), 3.20 (m, 2H), 3.38 (m, 2H), 3.46 (m, 2H), 7.45 (m, 1H), 7.56 (m, 2H), 7.82 (m, 2H), 8.72 (s, 1H); m/z 522.1 (APCI pos) (100%) [M + H]. |
| E127 | 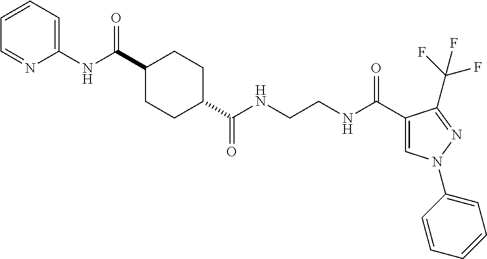 | trans-N1-(2- (1-phenyl-3- (trifluoromethyl)- 1H- pyrazole-4- carboxamido) ethyl)-N4- (pyridin-2- yl)cyclohexane- 1,4- dicarboxamide | Solid (55%); ¹H NMR (400 MHz, CD₃OD) δ 1.55 (m, 4H), 1.95 (m, 4H), 2.22 (m ,1H), 2.45 (m, 1H), 3.40 (m, 2H), 3.45 (m, 2H), 6.58 (m, 1H), 7.08 (m, 1H), 7.45 (m, 1H), 7.57 (m, 2H), 7.74 (m, 1H), 7.83 (m, 1H), 8.05 (m, 1H), 8.26 (m, 1H), 8.72 (m, 1H); m/z 529.2 (APCI pos) (100%) [M + H]. |
| E128 | 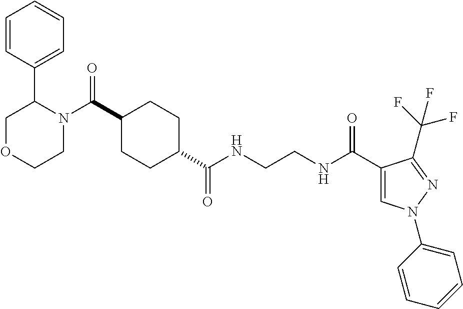 | 1-phenyl-N- (2-(trans-4- (3- phenylmorpholine- 4- carbonyl)cyclo- hexanecarbox- amido)ethyl)- 3- (trifluoromethyl)- 1H- pyrazole-4- carboxamide | Solid (11%); ¹H NMR (400 MHz, CD₃OD (δ 1.55 (m, 5H), 1.82 (m, 4H), 2.20 (m, 1H), 2.66 (m, 1H), 3.38 (m, 2H), 3.46 (m, 2H), 3.57 (m, 1H), 3.81 (m, 3H), 4.47 (m, 1H), 5.59 (m, 1H), 7.37 (m, 6H), 7.56 (m, 2H), 7.82 (m, 2H), 8.71 (s, 1H); m/z 598.3 (APCI pos) (100%) [M + H]. |
| E129 | 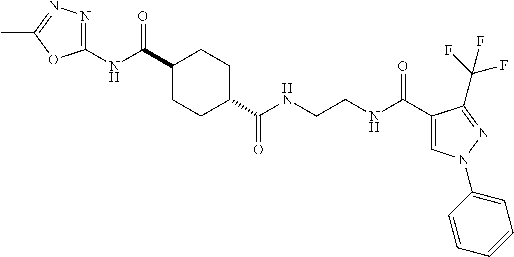 | trans-N1-(5- methyl- 1,3,4- oxadiazol-2- yl)-N4-(2- (1-phenyl-3- (trifluoromethyl)- 1H- pyrazole-4- carobxamido) ethyl)cyclo- hexane-1,4- dicarboxamide | Solid (30%); ¹H NMR (400 MHz, DMSO-d₆) δ 1.37 (m, 4H), 1.84 (m, 4H), 2.09 (m, 1H), 2.38 (m, 1H), 2.43 (s, 3H), 3.20 (m, 2H), 3.27 (m, 2H), 7.48 (m, 1H), 7.61 (m, 2H), 7.83 (m, 3H), 8.36 (m, 1H), 9.04 (s, 1H); m/z 534.1 (APCI pos) (100%) [M + H]. |
| E130 | 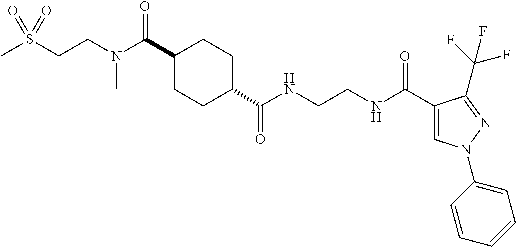 | trans-N1- methyl-N1- (2- methylsulfonyl) ethyl)- N4-(2-(1- phenyl-3- (trifluoromethyl)- 1H- pyrazole-4- carboxamido) ethyl)cyclo- hexane-1,4- dicarboxamide | Solid (45%); ¹H NMR (400 MHz, DMSO-d₆) δ 1.37 (m, 5H), 1.72 (m, 5H), 2.07 (m, 1H), 2.99 (s, 2H), 3.03 (s, 3H), 3.16 (m, 2H), 3.18 (m, 2H), 3.26 (m, 2H), 3.47 (m, 0.5H), 3.65 (m, 1H), 3.73 (m, 0.5H), 7.48 (m, 1H), 7.61 (m, 2H), 7.82 (m, 3H), 8.36 (m, 3H), 9.04 (s, 1H); m/z 572.0 (APCI pos) (100%) [M + H]. |
| E131 | 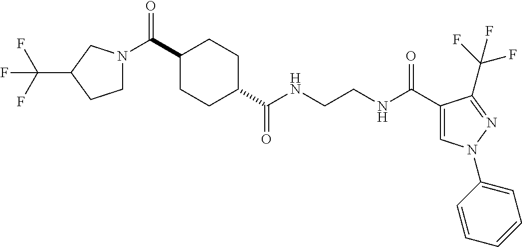 | 1-phenyl-3- (trifluoromethyl)- N-(2- (trans-4-(3- (trifluoromethyl)- pyrrolidine-1- carbonyl)cyclo- hexanecarbox- amido)ethyl)- 1H- pyrazole-4- carboxamide | Solid (12%); ¹H NMR (400 MHz, CD₃OD) δ 1.17 (m, 1H), 1.29 (s, 1H), 1.50 (m, 4H), 1.85 (m, 4H), 2.02- 2.30 (m, 3H), 2.48 (m, 1H), 3.39 (m, 2H), 3.48 (m, 3H), 3.66 (m, 2H), 7.45 (m, 1H), 7.56 (m, 2H), 7.81 (m, 2H), 8.72 (s, 1H); m/z 574.3 (APCI pos) (100%) [M + H]. |
| E132 | 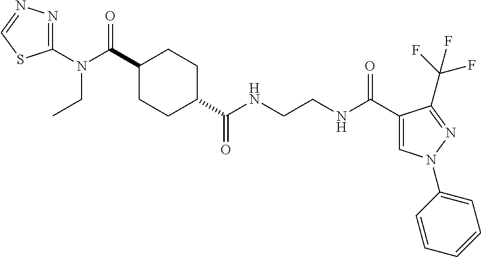 | trans-N1- ethyl-N4-(2- (1-phenyl-3- (trifluoromethyl)- 1H- pyrazole-4- carboxamido) ethyl)-N1- (1,3,4- thiadiazol- 2- yl)cyclohexane- 1,4- dicarboxamide | Solid (37%); ¹H NMR NMR (400 MHz, DMSO-d₆) δ 1.32 (t, 3H, J = 7.03 Hz), 1.52 (m, 4H), 1.84 (m, 4H), 2.16 (m, 1H), 2.91 (m, 1H), 3.25 (m, 4H), 4.32 (q, 2H, J = 7.03 Hz), 7.48 (m, 1H), 7.61 (m, 2H), 7.82 (m, 2H), 7.89 (m, 1H), 8.37 (m, 1H), 8.60 (s, 1H), 9.04 (s, 1H); m/z 564.0 (APCI pos) (100%) [M + H]. |
| E133 | 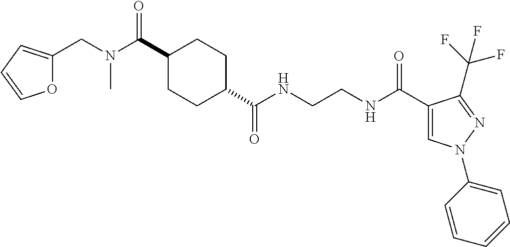 | trans-N1- (furan-2- ylmethyl)- N1-methyl- N4-(2-(1- phenyl-3- (trifluoromethyl)- 1H- pyrazole-4- carboxamido) ethyl)cyclo- hexane-1,4- dicarboxamide | Solid (39%); ¹H NMR (400 MHz, CD₃OD) δ 1.52 (m, 4H), 1.82 (m, 4H), 2.21 (m, 1H), 2.66 (m, 0.5H), 2.83 (m, 0.5H), 2.90 (s, 1.5H), 3.06 (s, 1.5H), 3.39 (m, 2H), 3.46 (m, 2H), 4.54 (s, 1H), 4.59 (s, 1H), 6.29 (m, 2H), 7.45 (m, 2H), 7.56 (m, 2H), 7.82 (m, 2H), 8.71 (s, 1H); m/z 546.3 (APCI pos) (100%) [M + H]. |
| E134 | 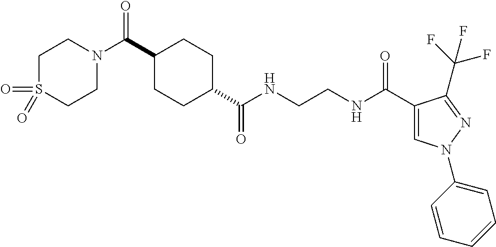 | N-(2-(trans- 4-((1,1- dioxidothiomor- pholin-4- yl)carbonyl) cyclohexanecar- boxamido)ethyl)- 1- phenyl-3- (trifluoromethyl)- 1H- pyrazole-4- carboxamide | Solid (55%); ¹H NMR (400 MHz, DMSO-d₆) δ 1.38 (m, 4H), 1.73 (m, 4H), 2.07 (m ,1H), 2.64 (m, 1H), 3.07 (br, 2H), 3.18 (m, 4H), 3.26 (m, 2H), 3.87 (m, 4H), 7.48 (m, 1H), 7.61 (m, 2H), 7.82 (m, 3H), 8.35 (m, 1H), 9.04 (s, 1H); m/z 570.1 (APCI pos) (100%) [M + H]. |
| E165 | 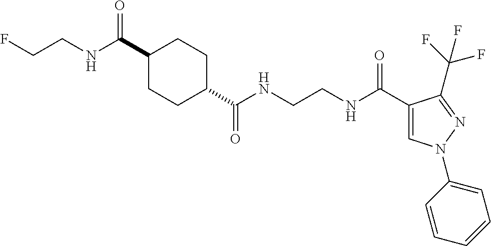 | trans-N1-(2- fluoroethyl)- N4-(2-(1- phenyl-3- (trifluoromethyl)- 1H- pyrazole-4- carboxamido) ethyl)cyclo- hexane-1,4- dicarboxamide | Solid (39%); ¹H NMR (400 MHz, DMSO-d₆) δ 1.33 (m, 4H), 1.74 (m, 4H), 2.06 (m, 1H), 3.20 (m, 2H), 3.27 (m, 4H), 3.34 (m, 1H), 4.33 (t, 1H, J = 5.1 Hz), 4.44 (t, 1H, J = 5.1 Hz), 7.48 (m, 1H), 7.61 (m, 2H), 7.82 (m, 3H), 7.98 (m, 1H), 8.35 (m, 1H), 9.04 (s, 1H); m/z (APCI pos) 498.1 (10%) [M + H]. |
| E166 | 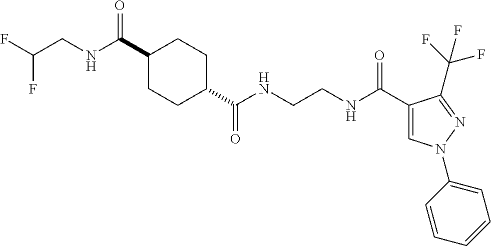 | trans-N1- (2,2- difluoroethyl)- N4-(2-(1- phenyl-3- (trifluoromethyl)- 1H- pyrazole-4- carboxamido) ethyl)cyclo- hexane-1,4- dicarboxamide | Solid (37%); ¹H NMR (400 MHz, DMSO-d₆) δ 1.33 (m, 4H), 1.75 (m, 4H), 2.08 (m, 2H), 3.19 (m, 2H), 3.26 (m, 2H), 3.43 (m, 2H), 5.82 (m, 0.2H), 5.96 (m, 0.6H), 6.10 (m, 0.2H), 7.48 (m, 1H), 7.61 (m, 2H), 7.82 (m, 3H), 8.14 (m, 1H), 8.35 (m, 1H), 9.03 (m, 1H); m/z (APCI pos) 516.1 (40%) [M + H]. |
| E177 | 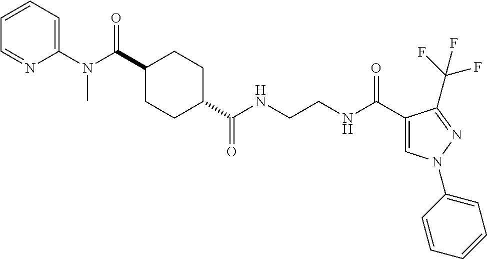 | trans-N1- methyl-N4- (2-(1- phenyl-3- (trifluoromethyl)- 1H- pyrazole-4- carboxamido) ethyl)-N1- (pyridin-2- yl)cyclohexane- 1,4- dicarboxamide | Solid (29%); ¹H NMR (400 MHz, DMSO-d₆) δ 1.13 (m, 2H), 1.37 (m, 2H), 1.70 (m, 4H), 2.04 (m, 1H), 2.31 (m, 1H), 3.16 (m, 2H), 3.21 (s, 3H), 3.23 (m, 2H), 7.34 (m, 1H), 7.46 (m, 2H), 7.61 (m, 2H), 7.79 (m, 3H), 7.91 (m, 1H), 8.32 (m, 1H), 8.49 (m, 1H), 9.01 (s, 1H); m/z (APCI pos) 543.2 (100%) [M + H]. |

### Example E135

### 4-(Cyclopropanecarboxamido)-N-(2-(1-phenyl-3-(trifluoromethyl)-1H-pyrazole-4-carboxamido)ethyl)piperidine-1-carboxamide

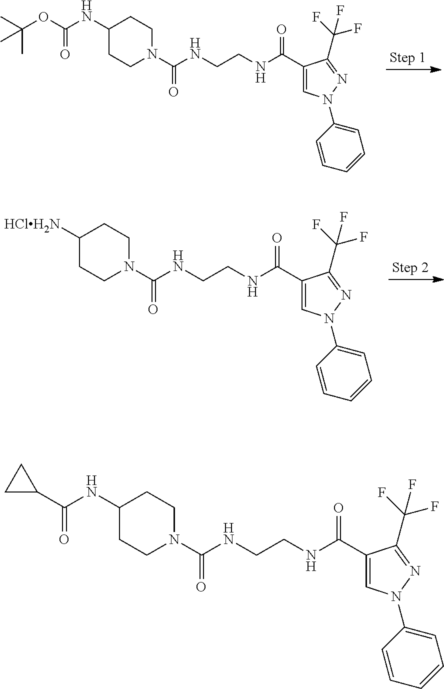

### Step 1

Following the procedure described in example E5,4-amino-N-(2-(1-phenyl-3-(trifluoromethyl)-1H-pyrazole-4-carboxamido)ethyl)piperidine-1-carboxamide hydrochloride was obtained as a white solid (84%) from tert-butyl 1-(2-(1-phenyl-3-(trifluoromethyl)-1H-pyrazole-4-carboxamido)ethylcarbamoyl)piperidin-4-ylcarbamate: ¹H NMR (400 MHz, DMSO-d₆) δ 1.38 (m, 2H), 1.83 (m, 2H), 2.73 (m, 2H), 3.20 (m, 3H), 3.28 (m, 2H), 3.99 (m, 2H), 6.85 (m, 1H), 7.48 (m, 1H), 7.61 (m, 2H), 7.83 (m, 2H), 8.00 (br, 2H), 8.56 (m, 1H), 9.28 (s, 1H); m/z (APCI pos) 425.1 (100%) [M+H].

### Step 2

### General Procedure for Preparation of TFP Library

To a Jones tube were added the appropriate TFP resin (1.2 eq for amine) and MP-carbonate (0.0829 g, 0.260 mmol) followed by 4-amino-N-(2-(1-phenyl-3-(trifluoromethyl)-1H-pyrazole-4-carboxamido)ethyl)piperidine-1-carboxamide hydrochloride (0.100 g, 0.217 mmol) and DIPEA (0.151 mL, 0.868 mmol) in THF/DMF (1:1, 400 μl). The tube was placed on an orbital shaker and agitated at 70 rpm for 48 hours. The tubes were then drained into another Jones Tube containing ˜2 mL of silica gel to remove the ubiquitous baseline impurity. The pad was then rinsed with 10% MeOH/DCM until eluant showed no UV abrorbance. The tubes were then concentrated on a Genevac vacuum centrifuge. The crude products were then purified by chromatography on silica gel (5% MeOH/DCM).

Compounds of the following structures were prepared from the corresponding TFP resins (carboxylates or sulfonyls), using a similar method to that described above.

| Ex # | Structure | Name | Physical Data |
|---|---|---|---|
| E135 | 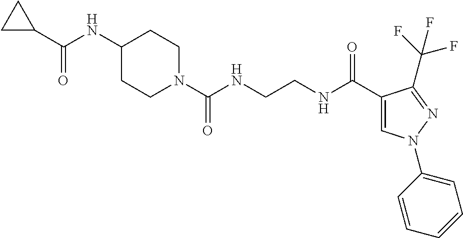 | 4- (cyclopropane carboxamido)- N-(2-(1- phenyl-3- (trifluorometh- yl)-1H- pyrazole-4- carboxamido) ethyl)piper- idine-1- carboxamide | Solid (51%); ¹H NMR (400 MHz, DMSO-d₆) δ 0.64 (m, 4H), 1.27 (m, 4H), 1.50 (m, 1H), 1.68 (m, 2H), 3.13 (m, 1H), 3.17 (m, 2H), 3.28 (m, 2H), 3.87 (m, 2H), 6.70 (m, 1H), 7.48 (m, 1H), 7.61 (m, 2H), 7.83 (m, 2H), 7.98 (m, 1H), 8.47 (m, 1H), 9.14 (s, 1H); m/z 493.1 (APCI pos) (100%) [M + H]. |
| E119 | 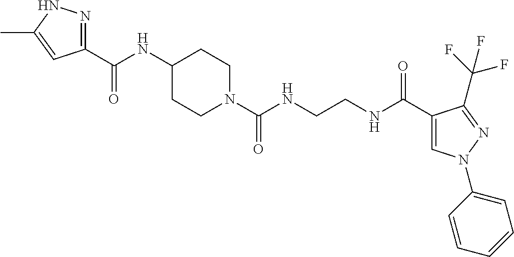 | 4-(5-methyl- 1H-pyrazole- 3- carboxamido)- N-(2-(1- phenyl-3- (trifluorometh- yl)-1H- pyrazole-4- carboxamido) ethyl)piper- idine-1- carboxamide | Solid (31%); ¹H NMR (400 MHz, DMSO-d₆) δ 1.44 (m, 2H), 1.68 (m, 2H), 2.25 (s, 3H), 2.75 (m, 2H), 3.19 (m, 2H), 3.29 (m, 2H), 3.93 (m, 3H), 6.35 (s, 1H), 6.67 (m, 1H), 7.47 (m, 1H), 7.59 (m, 2H), 7.81 (m, 3H), 8.41 (m, 1H), 9.06 (s, 1H); m/z 533.3 (APCI pos) (100%) [M + H]. |
| E120 | 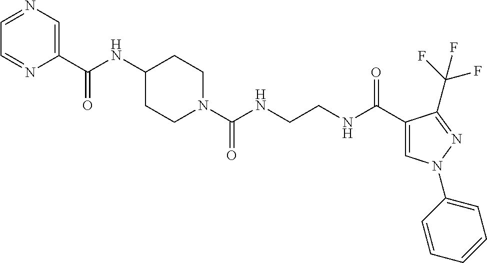 | N-(1-((2-(1- phenyl-3- (trifluorometh- yl)-1H- pyrazole-4- carboxamido) ethyl)carbamoyl) piperidin- 4- yl)pyrazine- 2-carboxamide | Solid (26%); ¹H NMR (400 MHz, CD₃OD) δ 1.56 (m, 2H), 1.92 (m, 2H), 2.96 (m, 2H), 3.38 (m, 2H), 3.47 (m, 2H), 4.06 (m, 3H), 7.44 (m, 1H), 7.55 (m, 2H), 7.81 (m, 2H), 8.66 (m, 1H), 8.74 (m, 1H), 8.76 (m, 1H), 9.21 (m, 1H); m/z 531.0 (APCI pos) (60%) [M + H]. |
| E121 | 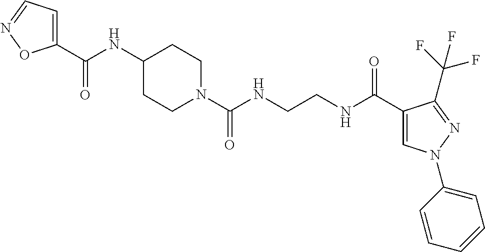 | 4-(isoxazole- 5- carboxamido)- N-(2-(1- phenyl-3- (trifluorometh- yl)-1H- pyrazole-4- carboxamido) ethyl)piper- idine-1- carboxamide | Solid (51%); ¹H NMR (400 MHz, CD₃OD) δ 1.53 (m, 2H), 1.91 (m, 2H), 2.92 (m, 2H), 3.38 (m, 2H), 3.47 (m, 2H), 4.06 (m, 3H), 6.94 (d, 1H, J = 1.95 Hz), 7.45 (m, 1H), 7.56 (m, 2H), 7.82 (m, 2H), 8.49 (d, 1H, J = 1.95 Hz), 8.74 (m, 1H); m/z 520.1 (APCI pos) (100%) [M + H]. |
| E136 | 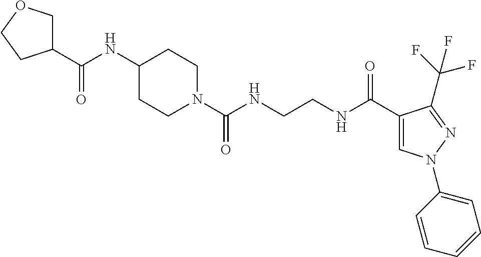 | N-(2-(1- phenyl-3- (trifluorometh- yl)-1H- pyrazole-4- carboxamido) ethyl)-4- (tetrahydro- furan-3- carboxamido) piperidine-1- carboxamide | Solid (78%); ¹H NMR (400 MHz, DMSO-d₆) δ 1.26 (m, 4H), 1.66 (m, 2H), 1.99 (m, 2H), 2.78 (m, 3H), 2.87 (m, 1H), 3.16 (m, 3H), 3.28 (m, 2H), 3.68 (m, 1H), 3.85 (m, 2H), 6.74 (m, 1H), 7.47 (m, 1H), 7.61 (m, 2H), 7.83 (m, 2H), 7.88 (m, 1H), 8.51 (m, 1H), 9.21 (s, 1H); m/z 523.1 (APCI pos) (100%) [M + H]. |
| E137 | 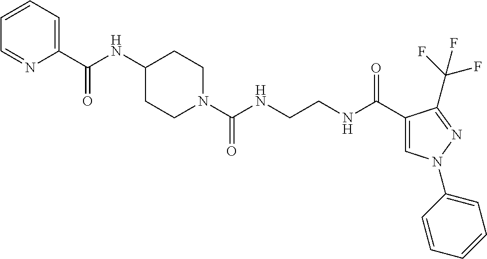 | N-(1-((2-(1- phenyl-3- (trifluorometh- yl)-1H- pyrazole-4- carboxamido) ethyl)carbamoyl) piperidin- 4- yl)picolinamide | Solid (16%); ¹H NMR (400 MHz, CD₃OD) δ 1.56 (m, 2H), 1.92 (m, 2H), 2.97 (m, 2H), 3.39 (m, 2H), 3.48 (m, 2H), 4.05 (m, 3H), 7.44 (m, 1H), 7.54 (m, 3H), 7.80 (m, 2H), 7.94 (m, 1H), 8.07 (m, 1H), 8.60 (m, 1H), 8.75 (s, 1H); m/z 530.3 (APCI pos) (100%) [M + H]. |
| E138 | 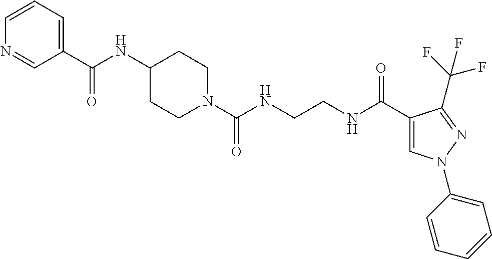 | N-(1-((2-(1- phenyl-3- (trifluorometh- yl)-1H- pyrazole-4- carboxamido) ethyl)carbamoyl) piperidin- 4- yl)nicotinamide | Solid (15%); ¹H NMR (400 MHz, CD₃OD) δ 1.53 (m, 2H), 1.95 (m, 2H), 2.94 (m, 2H), 3.38 (m, 2H), 3.47 (m, 2H), 4.07 (m, 3H), 7.45 (m, 1H), 7.54 (m, 3H), 7.82 (m, 2H), 8.22 (m, 1H), 8.67 (m, 1H), 8.75 (m, 1H), 8.94 (m, 1H); m/z 530.1 (APCI pos) (100%) [M + H]. |
| E139 | 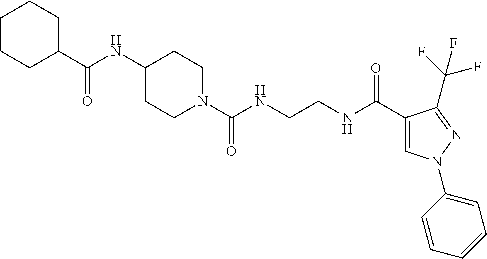 | 4- (cyclohexane- carboxamido)- N-(2-(1- phenyl-3- (trifluorometh- yl)-1H- pyrazole-4- carboxamido) ethyl)piper- idine-1- carboxamide | Solid (38%); ¹H NMR (400 MHz, DMSO-d₆) δ 1.24 (m, 6H), 1.65 (m, 7H), 2.04 (m, 1H), 2.77 (m, 2H), 3.18 (m, 2H), 3.13 (m, 1H), 3.28 (m, 2H), 3.66 (m, 1H), 3.85 (m, 2H), 6.68 (m, 1H), 7.48 (m, 1H), 7.60 (m, 3H), 7.82 (m, 2H), 8.45 (m, 1H), 9.11 (m, 1H); m/z 535.3 (APCI pos) (100%) [M − H]. |
| E140 | 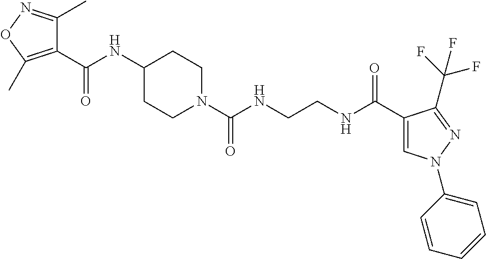 | 4-(3,5- dimethylisoxa- zole-4- carboxamido)- N-(2-(1- phenyl-3- (trifluorometh- yl)-1H- pyrazole-4- carboxamido) ethyl)piper- idine-1- carboxamide | Solid (61%); ¹H NMR (400 MHz, DMSO-d₆) δ 1.36 (m, 2H), 1.75 (m, 2H), 2.73 (s, 3H), 2.83 (m, 2H), 2.89 (s, 3H), 3.20 (m, 2H), 3.29 (m, 2H), 3.89 (m, 3H), 6.76 (m, 1H), 7.47 (m, 1H), 7.61 (m, 2H), 7.82 (m, 2H), 7.90 (m, 1H), 8.50 (m, 1H), 9.19 (s, 1H); m/z 548.3 (APCI pos) (100%) [M + H]. |
| E141 | 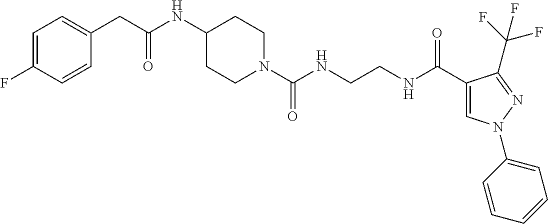 | 4-(2-(4- fluorophenyl) acetamido)-N- (2-(1-phenyl- 3- (trifluorometh- yl)-1H- pyrazole-4- carboxamido) ethyl)piper- idine-1- carboxamide | Solid (45%); ¹H NMR (400 MHz, DMSO-d₆) δ 1.23 (m, 2H), 1.67 (m, 2H), 2.79 (m, 2H), 3.18 (m, 2H), 3.28 (m, 2H), 3.32 (s, 2H), 3.68 (m, 1H), 3.83 (m, 2H), 6.65 (m, 1H), 7.11 (m, 2H), 7.26 (m, 2H), 7.48 (m, 1H), 7.61 (m, 2H), 7.81 (m, 2H), 8.01 (m, 1H), 8.40 (m, 1H), 9.06 (s, 1H); m/z 561.1 (APCI pos) (100%) [M + H]. |
| E142 | 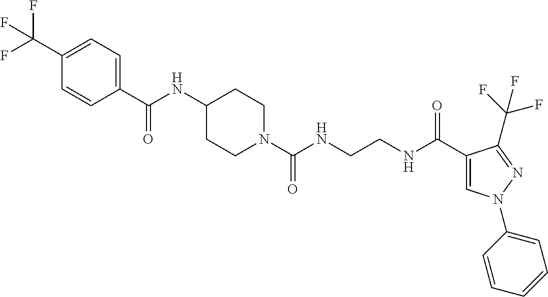 | N-(2-(1- phenyl-3- (trifluorometh- yl)-1H- pyrazole-4- carboxamido) ethyl)-4-(4- (trifluorometh- yl)benzamido) piperidine- 1-carboxamide | Solid (44%); ¹H NMR (400 MHz, DMSO-d₆) δ 1.46 (m, 2H), 1.78 (m, 2H), 2.81 (m, 2H), 3.22 (m, 2H), 3.30 (m, 2H), 3.98 (m, 3H), 6.79 (m, 1H), 7.47 (m, 1H), 7.60 (m, 2H), 7.84 (m, 4H), 8.04 (m, 2H), 8.53 (m, 2H), 9.22 (s, 1H); m/z 597.1 (APCI pos) (100%) [M + H]. |
| E143 | 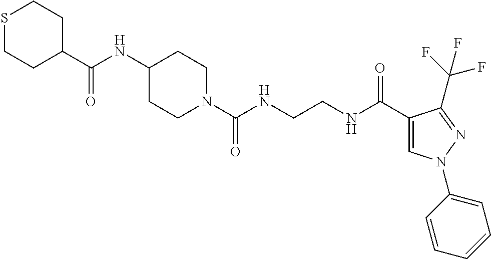 | N-(2-(1- phenyl-3- (trifluorometh- yl)-1H- pyrazole-4- carboxamido) ethyl)-4- (tetrahydro- 2H-thiopyran- 4- carboxamido) piperidine-1- carboxamide | Solid (48%); ¹H NMR (400 MHz, DMSO-d₆) δ 1.63 (m, 4H), 1.92 (m, 2H), 2.13 (m, 1H), 2.58 (m, 4H), 2.77 (m, 2H), 3.16 (m, 3H), 3.27 (m, 2H), 3.63 (m, 2H), 3.83 (m, 2H), 6.67 (m, 1H), 7.48 (m, 1H), 7.63 (m, 3H), 7.81 (m, 2H), 8.44 (m, 1H), 9.11 (s, 1H); m/z 553.1 (APCI pos) (100%) [M + H]. |
| E144 | 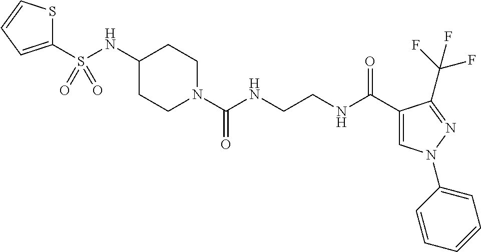 | N-(2-(1- phenyl-3- (trifluorometh- yl)-1H- pyrazole-4- carboxamido) ethyl)-4- (thiophene-2- sulfonamido) piperidine-1- carboxamide | Solid (1%); ¹H NMR (400 MHz, CD₃OD) δ 1.37 (m, 2H), 1.69 (m, 2H), 2.86 (m, 2H), 3.34 (m, 3H), 3.43 (m, 2H), 3.82 (m, 2H), 7.12 (m, 1H), 7.45 (m, 1H), 7.56 (m, 2H), 7.60 (m, 1H), 7.75 (m, 1H), 7.80 (m, 2H), 8.71 (m, 1H); m/z 571.0 (APCI pos) (100%) [M + H]. |

### Example E148

### N-(2-(trans-4-Benzoylcyclohexanecarboxamido)ethyl)-1-(3-fluorophenyl)-3-(trifluoromethyl)-1H-pyrazole-4-carboxamide

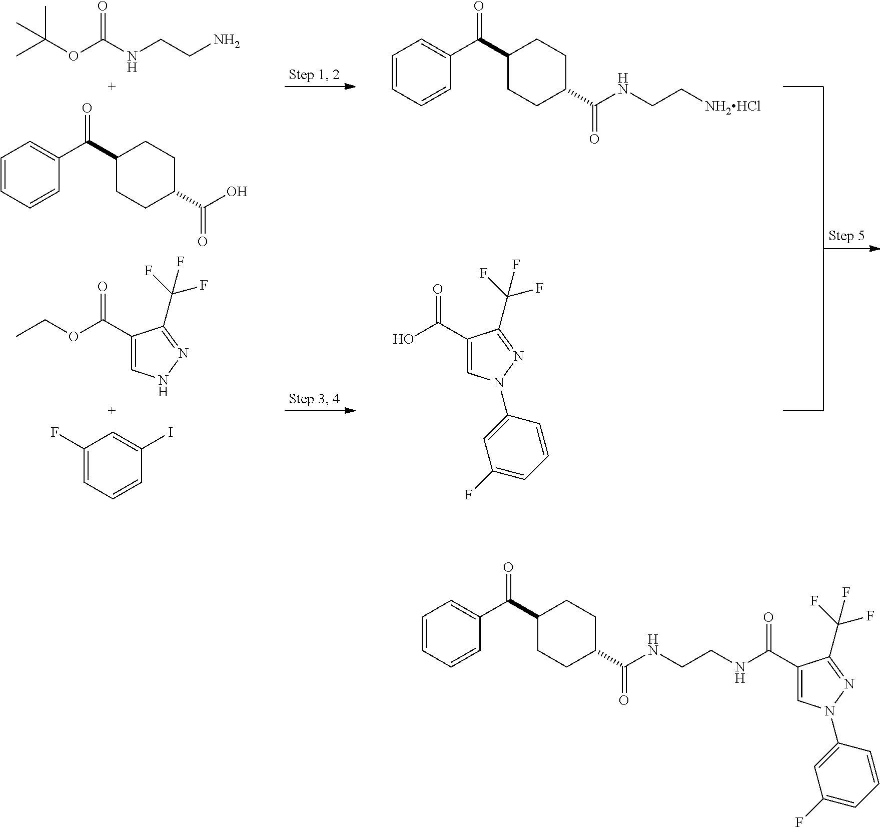

### Step 1

To a solution of trans-4-benzoylcyclohexanecarboxylic acid (1.0 g, 4.3 mmol), EDAC.HCl (0.91 g, 4.7 mmol), and HOBt.H₂O (0.73 g, 4.7 mmol) in DMF (20 mL) was added tert-butyl 2-aminoethylcarbamate (0.76 g, 4.7 mmol). The mixture was stirred for 16 hours, the solvent was removed under vacuum, and the residue was partitioned between AcOEt and water. The organic layer was washed with brine, dried (sodium sulfate), filtered and concentrated in vacuo, and the crude product was purified by chromatography on silica gel (3% MeOH/DCM) to give tert-butyl 2-(trans-4-benzoylcyclohexanecarboxamido)ethylcarbamate (1.4 g, 87% yield) as a solid: ¹H NMR (400 MHz, DMSO-d₆) δ 1.45 (s, 9H), 1.59 (m, 6H), 2.05 (m, 4H), 3.32 (m, 4H), 7.47 (m, 2H), 7.56 (m, 1H), 7.93 (m, 2H).

### Step 2

To a solution of tert-butyl 2-(trans-4-benzoylcyclohexanecarboxamido)ethylcarbamate (1.4 g, 3.74 mmol) in DCM was added HCl (0.935 mL, 3.74 mmol) (4M in dioxane). The mixture was stirred for 3 hours, and concentrated in vacuo, and the residue was suspended in ether and filtered to give trans-N-(2-aminoethyl)-4-benzoylcyclohexanecarboxamide hydrochloride (1.26 g, quant.) as a solid: ¹H NMR (400 MHz, DMSO-d₆) δ 1.36 (m, 2H), 1.55 (m, 2H), 1.87 (m, 4H), 2.13 (m, 1H), 2.84 (m, 2H), 3.29 (m, 2H), 3.41 (m, 1H), 7.53 (m, 2H), 7.64 (m, 1H), 7.97 (m, 4H), 8.07 (m, 1H); m/z (APCI pos) 275.1 (100%) [M+H].

### Step 3

General procedure (No. 1) for Copper-Mediated Coupling of Ethyl 3-(trifluoromethyl)-1H-pyrazole-4-carboxylate and Aryl Halides

According to the method of Cristau et al. (Eur. J. Org. Chem. 2004, 695-709), to a 350 mL sealed tube flushed vigorously with nitrogen were added ethyl 3-(trifluoromethyl)-1H-pyrazole-4-carboxylate (416 mg, 2.0 mmol), 1-fluoro-3-iodobenzene (667 mg, 3.0 mmol), cesium carbonate (1.3 g, 4.0 mmol), copper(II) oxide (14 mg, 0.1 mmol), and 2-hydroxybenzaldehyde oxime (55 mg, 0.4 mmol), followed by degassed (argon) DMF (1.2 mL). The mixture was stirred for 24 hours at 110° C., cooled to room temperature, and filtered through a short silica pad which was rinsed with AcOEt. The filtrate was concentrated in vacuo to give ethyl 1-(3-fluorophenyl)-3-(trifluoromethyl)-1H-pyrazole-4-carboxylate (0.480 g, 79%) as a solid: ¹H NMR (400 MHz, CDCl₃) δ 1.39 (t, 3H, J=7.0 Hz), 4.37 (q, 2H, J=7.0 Hz), 7.13 (m, 1H), 7.51 (m, 3H), 8.49 (m, 1H).

Compounds of the following structures were prepared from the corresponding aryl halides, using a similar method to that described above.

| Structure | Name | Physical Data |
|---|---|---|
| 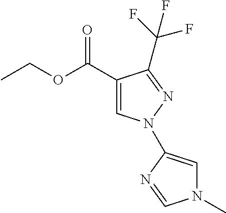 | ethyl 1-(1- methyl-1H- imidazol-4- yl)-3- (trifluorometh- yl)-1H- pyrazole-4- carboxylate | Solid (82%); ¹H NMR (400 MHz, CDCl₃) δ 1.36 (m, 3H), 3.71 (s, 1H), 3.76 (s, 2H), 4.34 (m, 2H), 7.37 (bs, 1H), 8.25 (br, 1H), 8.67 (br, 1H). |
| 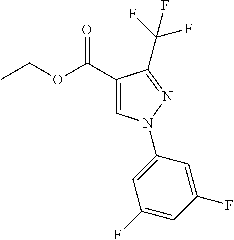 | ethyl 1-(3,5- difluorophenyl)- 3- (trifluorometh- yl)-1H- pyrazole-4- carboxylate | Solid (69%); ¹H NMR (400 MHz, CDCl₃) δ 1.39 (m, 3H), 4.38 (m, 2H), 6.88 (m, 1H), 7.26 (m, 1H), 7.34 (m, 1H), 8.52 (br, 1H). |
| 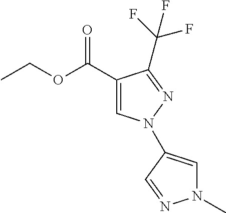 | ethyl 1-(1- methyl-1H- pyrazol-4- yl)-3- (trifluorometh- yl)-1H- pyrazole-4- carboxylate | Solid (100%); ¹H NMR (400 MHz, CDCl₃) δ 1.38 (t, 3H, J = 7.0 Hz), 3.92 (s, 3H), 4.36 (q, 2H), J = 7.0 Hz), 7.52 (m, 2H), 8.23 (m, 1H). |
| 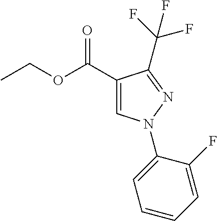 | ethyl 1-(2- fluorophenyl)- 3- (trifluorometh- yl)-1H- pyrazole-4- carboxylate | Solid (25%); ¹H NMR (400 MHz, CDCl₃) δ 1.39 (t, 3H, J = 7.0 Hz), 4.37 (q, 2H, J = 7.0 Hz), 7.13 (m, 1H), 7.51 (m, 3H), 8.49 (m, 1H). |
| 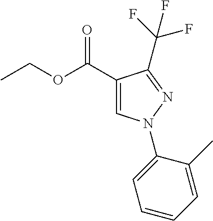 | ethyl 1-o- tolyl-3- (trifluorometh- yl)-1H- pyrazole-4- carboxylate | Solid (9%); ¹H NMR (400 MHz, CDCl₃) δ 1.38 (m, 3H), 2.25 (s, 3H), 4.36 (m, 2H), 6.78 (m, 1H), 7.08 (m, 1H), 7.34 (m, 1H), 7.38 (m, 1H), 8.17 (m, 1H), |
| 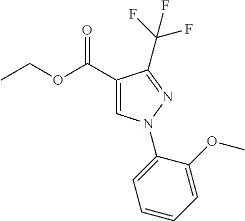 | ethyl 1-(2- methoxyphenyl)- 3- (trifluorometh- yl)-1H- pyrazole-4- carboxylate | Solid (4%); ¹H NMR (400 MHz, CDCl₃) δ 1.38 (t, 3H, J = 7.0 Hz), 3.92 (s, 3H), 4.36 (q, 2H, J = 7.0 Hz), 7.09 (m, 2H), 7.40 (m, 1H), 7.72 (m, 1H), 8.56 (m, 1H). |
| 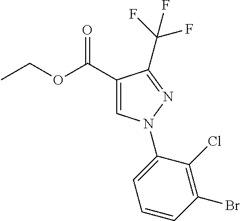 | ethyl 1-(3- bromo-2- chlorophenyl)- 3- (trifluorometh- yl)-1H- pyrazole-4- carboxylate | Solid (46%); ¹H NMR (400 MHz, CDCl₃) δ 1.38 (t, 3H, J = 7.0 Hz), 4.37 (q, 2H, J = 7.0 Hz), 7.31 (t, 1H, J = 7.8 Hz), 7.54 (m, 1H), 7.80 (m, 1H), 8.38 (m, 1H). |
| 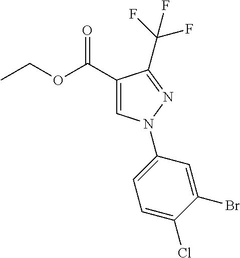 | ethyl 1-(3- bromo-4- chlorophenyl)- 3- (trifluorometh- yl)-1H- pyrazole-4- carboxylate | Solid (34%); ¹H NMR (400 MHz, CDCl₃) δ 1.39 (t, 3H, J = 7.0 Hz), 4.37 (q, 2H, J = 7.0 Hz), 6.99 (m, 1H), 7.40 (m, 2H), 8.46 (m, 1H). |

### Step 3

General Procedure (No. 2) for Copper-Mediated Coupling of ethyl 3-(trifluoromethyl)-1H-pyrazole-4-carboxylate and Aryl Halides

According to the procedure of Buchwald et al. (J. Org. Chem. 2004, 69, 5578), to a 50 mL sealed tube flushed with argon were added copper(I) iodide (0.02288 g, 0.1201 mmol), K₂CO₃ (0.6972 g, 5.045 mmol), and ethyl 3-(trifluoromethyl)-1H-pyrazole-4-carboxylate (0.500 g, 2.402 mmol). (1S,2S)−N1,N2-Dimethylcyclohexane-1,2-diamine (0.06834 g, 0.4805 mmol) and 2-bromophenol (0.3343 mL, 2.883 mmol) were then added along with 3 mL of toluene (degassed under argon). The tube was sealed and heated to 110° C. overnight, then cooled to room temperature and the mixture was filtered through celite to remove the solid. The filtrate was concentrated under vacuum and the residue was purified by chromatography (10% ether/DCM) to give ethyl 1-(2-hydroxyphenyl)-3-(trifluoromethyl)-1H-pyrazole-4-carboxylate (0.660 g, 91%) as a solid: ¹H NMR (400 MHz, CDCl₃) δ 1.40 (t, 3H, J=7.0 Hz), 4.39 (q, 2H, J=7.0 Hz), 7.00 (m, 1H), 7.16 (m, 1H), 7.31 (m, 1H), 7.42 (m, 1H), 8.56 (m, 1H).

Compounds of the following structures were prepared from the corresponding aryl halides, using a similar method to that described above.

| Structure | Name | Physical Data |
|---|---|---|
| 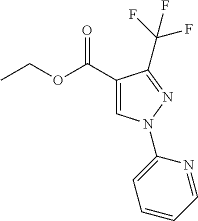 | ethyl 1- (pyridin-2- yl)-3- (trifluorometh- yl)-1H- pyrazole-4- carboxylate | Solid (87%); ¹H NMR (400 MHz, CDCl₃) δ 1.39 (t, 3H, J = 7.4 Hz), 4.37 (q, 2H, J = 7.0 Hz), 7.34 (m, 1H), 7.90 (m, 1H), 8.06 (m, 1H), 8.48 (m, 1H), 9.13 (m, 1H). |
| 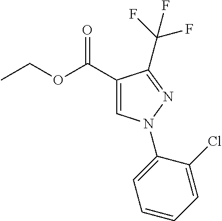 | ethyl 1-(2- chlorophenyl)- 3- (trifluorometh- yl)-1H- pyrazole-4- carboxylate | Solid (14%); ¹H NMR (400 MHz, CDCl₃) δ 1.38 (t, 3H, J = 7.0 Hz), 4.37 (q, 2H, J = 7.0 Hz), 7.18 (m, 1H), 7.31 (m, 1H), 7.44 (m, 1H), 7.58 (m, 1H), 8.41 (m, 1H). |

### Step 4

To a solution of ethyl 1-(3-fluorophenyl)-3-(trifluoromethyl)-1H-pyrazole-4-carboxylate (480 mg, 1.6 mmol) in EtOH (2 mL) and THF (2 mL) was added 2M sodium hydroxide (2.4 mL, 2.8 mmol). The mixture was stirred at room temperature for 4 hours then concentrated in vacuo. The residue was dissolved in water and washed with ether. The aqueous layer was then adjusted to pH ˜4 with 10% HCl. The resulting precipitate was collected by filtration and dried under vacuum to give 1-(3-fluorophenyl)-3-(trifluoromethyl)-1H-pyrazole-4-carboxylic acid (270 mg, 62% yield) as a powder: ¹H NMR (400 MHz, DMSO-d₆) δ 7.33 (m, H), 7.62 (m, 1H), 7.83 (m, 1H), 7.88 (m, 1H), 9.30 (m, 1H); m/z 272.9 (APCI neg) (100%) [M−H].

Compounds of the following structures were prepared from the corresponding esters, using a similar method to that described above.

| Structure | Name | Physical Data |
|---|---|---|
| 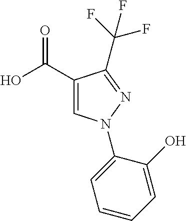 | 1-(2- hydroxyphenyl)- 3- (trifluorometh- yl)-1H- pyrazole-4- carboxylic acid | Solid (82%); ¹H NMR (400 MHz, DMSO- d₆) δ 6.97 (m, 1H), 7.10 (m, 1H), 7.32 (m, 1H), 7.58 (m, 1H), 8.78 (m, 1H), 10.63 (s, 1H), 13.10 (br, 1H). |
| 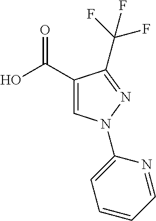 | 1-(pyridin-2- yl)-3- (trifluorometh- yl)-1H- pyrazole-4- carboxylic acid | Solid (94%); ¹H NMR (400 MHz, DMSO- d₆) δ 7.55 (m, 1H), 7.99 (d, 1H, J = 8.2 Hz), 8.11 (m, 1H), 8.59 (m, 1H), 9.09 (m, 1H), 13.33 (br, 1H). |
| 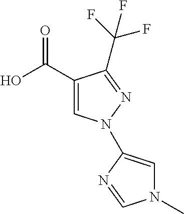 | 1-(1-methyl- 1H-imidazol- 4-yl)-3- (trifluorometh- yl)-1H- pyrazole-4- carboxylic acid | Solid (29%); ¹H NMR (400 MHz, DMSO- d₆) δ 3.72 (s, 3H), 7.60 (br, 1H), 7.70 (br, 1H), 8.67 (br, 1H), 13.14 (br, 1H); m/z 259.1 (APCI neg) (10%) [M − H]. |
| 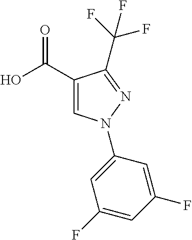 | 1-(3,5- difluorophenyl)- 3- (trifluorometh- yl)-1H- pyrazole-4- carboxylic acid | Solid (10%); m/z 290.9 (APCI neg) (100%) [M − H]. |
| 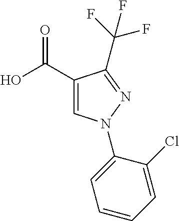 | 1-(2- chlorophenyl)- 3- (trifluorometh- yl)-1H- pyrazole-4- carboxylic acid | Solid (64%); ¹H NMR (400 MHz, DMSO- d₆) δ 7.61 (m, 2H), 7.75 (m, 2H), 8.91 (m, 1H); m/z 288.9 (APCI neg) (100%) [M − H]. |
| 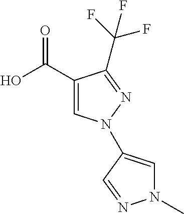 | 1-(1-methyl- 1H-pyrazol-4- yl)-3- (trifluorometh- yl)-1H- pyrazole-4- carboxylic acid | Solid (53%); m/z 258.9 (APCI neg) (100%) [M − H]. |
| 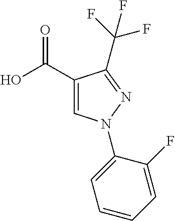 | 1-(2- fluorophenyl)- 3- (trifluorometh- yl)-1H- pyrazole-4- carboxylic acid | Solid (92%); ¹H NMR (400 MHz, DMSO- d₆) δ 7.32 (m, 1H), 7.62 (m, 1H), 7.85 (m, 2H), 9.30 (s, 1H); m/z 272.9 (APCI neg) (80%) [M − H]. |
| 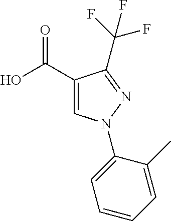 | 1-o-tolyl-3- (trifluorometh- yl)-1H- pyrazole-4- carboxylic acid | Solid (61%); ¹H NMR (400 MHz, DMSO- d₆) δ 2.23 (s, 3H), 7.41 (m, 4H), 8.50 (m, 1H); m/z 268.9 (APCI neg) (100%) [M − H]. |
| 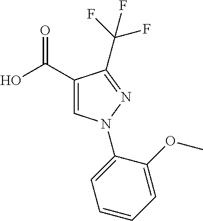 | 1-(2- methoxypheny)- 3- (trifluorometh- yl)-1H- pyrazole-4- carboxylic acid | Solid (44%); ¹H NMR (400 MHz, DMSO- d₆) δ 3.94 (s, 3H), 7.12 (m, 1H), 7.26 (m, 1H), 7.47 (m, 1H), 7.65 (m, 1H), 8.64 (m, 1H); m/z 284.9 (APCI neg) (100%) [M − H]. |
| 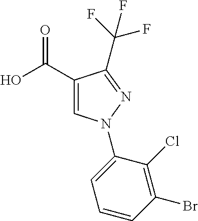 | 1-(3-bromo-2- chlorophenyl)- 3- (trifluorometh- yl)-1H- pyrazole-4- carboxylic acid | Solid (54%); ¹H NMR (400 MHz, DMSO- d₆) δ 7.44 (t, 1H, J = 8.2 Hz), 7.63 (m, 1H), 7.93 (m, 1H), 8.64 (m, 1H); m/z 368.8 (APCI neg) (100%) [M − H]. |
| 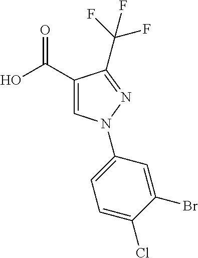 | 1-(3-bromo-4- chlorophenyl)- 3- (trifluorometh- yl)-1H- pyrazole-4- carboxylic acid | Solid (40%); ¹H NMR (400 MHz, DMSO- d₆) δ 7.70 (m, 1H), 7.88 (m, 1H), 8.27 (m, 1H), 8.97 (br, 1H); m/z 368.7 (APCI neg) (100%) [M − H]. |

### Step 5

To a solution of 1-(3-fluorophenyl)-3-(trifluoromethyl)-1H-pyrazole-4-carboxylic acid (64 mg, 0.23 mmol), EDAC.HCl (44 mg, 0.23 mmol), and HOBt.H₂O (31 mg, 0.23 mmol) in DMF were added trans-N-(2-aminoethyl)-4-benzoylcyclohexanecarboxamide hydrochloride (60 mg, 0.19 mmol) and DIPEA (0.067 mL, 0.39 mmol). The mixture was stirred for 16 hours, the solvent was removed under vacuum, and the residue was partitioned between AcOEt and water. The organic layer was washed with brine, dried (sodium sulfate), filtered and concentrated in vacuo, and the crude product was purified by chromatography on silica gel (3% MeOH/DCM) to give N-(2-(trans-4-benzoylcyclohexanecarboxamido)ethyl)-1-(3-fluorophenyl)-3-(trifluoromethyl)-1H-pyrazole-4-carboxamide (45 mg, 44%) as a solid: ¹H NMR (400 MHz, DMSO-d₆) δ 1.34 (m, 2H), 1.56 (m, 2H), 1.84 (m, 4H), 2.11 (m, 1H), 3.21 (m, 2H), 3.28 (m, 2H), 3.39 (m, 1H), 7.35 (m, 1H), 7.53 (m, 3H), 7.67 (m, 3H), 7.87 (m, 1H), 7.97 (m, 2H), 8.37 (m, 1H), 9.09 (s, 1H); m/z (APCI pos) 531.1 (100%) [M+H].

Compounds of the following structures were prepared from the corresponding acids, using a similar method to that described above.

| Ex # | Structure | Name | Physical Data |
|---|---|---|---|
| E149 | 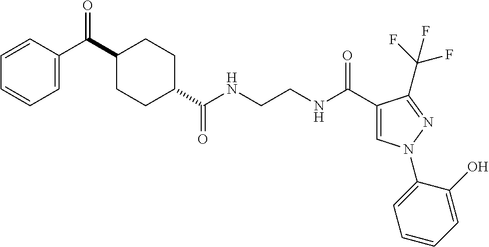 | N-(2-(trans- 4- benzoylcyclo- hexanecarbox- amido)ethyl)-1- (2- hydroxyphenyl)- 3- (trifluorometh- yl)-1H- pyrazole-4- carboxamide | Solid (10%); ¹H NMR (400 MHz, CD₃OD) δ 1.49 (m, 2H), 1.63 (m, 2H), 1.94 (m, 4H), 2.22 (m, 1H), 3.42 (m, 5H), 6.99 (m, 1H), 7.05 (m, 1H), 7.28 (m, 1H), 7.49 (m, 2H), 7.61 (m, 2H), 7.64 (m, 2H), 8.72 (m, 1H); m/z (APCI pos) 529.1 (100%) [M + H]. |
| E150 | 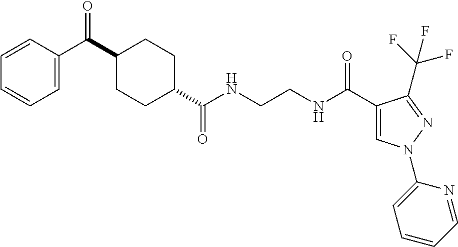 | N-(2-(trans- 4- benzoylcyclo- hexanecarbox- amido)ethyl)-1- (pyridin-2- yl)-3- (trifluorometh- yl)-1H- pyrazole-4- carboxamide | Solid (10%); ¹H NMR (400 MHz, DMSO-d₆) δ 1.37 (m, 2H), 1.59 (m, 2H), 1.84 (m, 4H), 2.12 (m, 1H), 2.51 (s, 2H), 3.25 (m, 4H), 3.40 (m, 1H), 7.53 (m, 3H), 7.63 (m, 1H), 7.89 (m, 1H), 7.98 (m, 2H), 8.11 (m, 1H), 8.58 (m, 1H), 9.38 (s, 1H); m/z (APCI pos) 514.1 (100%) [M + H]. |
| E157 | 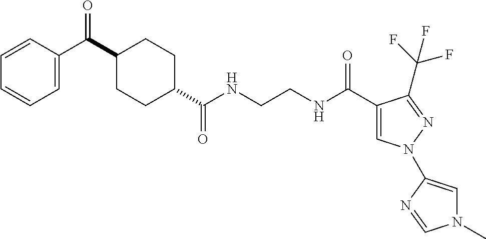 | N-(2-(trans- 4- benzoylcyclo- hexanecarbox- amido)ethyl)-1- (1-methyl-1H- imidazol-4- yl)-3- (trifluorometh- yl)-1H- pyrazole-4- carboxamide | ¹H NMR (400 MHz, DMSO- d₆) δ 1.35 (m, 2H), 1.55 (m, 2H), 1.83 (m, 4H), 2.11 (m, 1H), 3.22 (m, 4H), 3.38 (m, 1H), 3.72 (s, 3H), 7.51-7.56 (m, 3H), 7.63 (m, 1H), 7.70 (m, 1H), 7.86 (t, 1H, J = 5.9 Hz), 7.96 (m, 2H), 8.42 (t, 1H, J = 5.5 Hz), 8.88 (m, 1H); m/z (APCI pos) 517.1 (100%) [M + H]. |
| E158 | 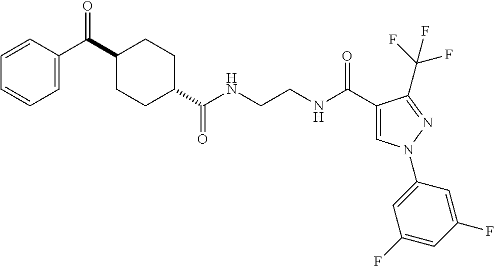 | N-(2-(trans- 4- benzoylcyclo- hexanecarbox- amido)ethyl)-1- (3,5- difluorophenyl)- 3- (trifluorometh- yl)-1H- pyrazole-4- carboxamide | ¹H NMR (400 MHz, DMSO- d₆) δ 1.33 (m, 2H), 1.55 (m, 2H), 1.84 (m, 4H), 2.11 (m, 1H), 3.21 (m, 2H), 3.28 (m, 2H), 3.40 (m, 1H), 7.43 (m, 1H), 7.53 (m, 2H), 7.63 (m, 3H), 7.87 (m, 1H), 7.96 (m, 2H), 8.37 (t, 1H, J = 5.5 Hz), 9.13 (s, 1H); m/z (APCI pos) 549.1 (100%) [M + H]. |
| E164 | 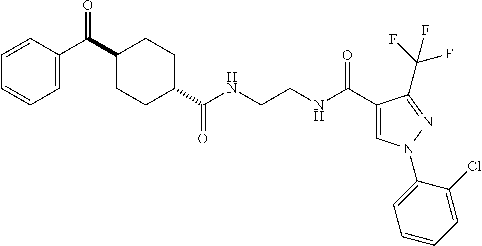 | N-(2-(trans- 4- benzoylcyclo- hexanecarbox- amido)ethyl)-1- (2- chlorophenyl)- 3- (trifluorometh- yl)-1H- pyrazole-4- carboxamide | ¹H NMR (400 MHz, CD₃OD) δ 1.46 (m, 2H), 1.62 (m, 2H), 1.93 (m, 4H), 2.21 (m, 1H), 3.36 (m, 1H), 3.39 (m, 2H), 3.47 (m, 2H), 7.48- 7.69 (m, 7H), 7.96 (m, 2H), 8.47 (m, 1H); m/z (APCI pos) 547.1 (100%) [M + H]. |
| E176 | 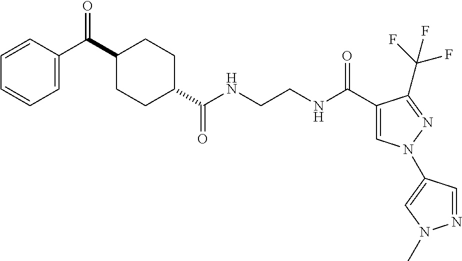 | N-(2-(trans- 4- benzoylcyclo- hexanecarbox- amido)ethyl)-1- (1-methyl-1H- pyrazol-4- yl)-3- (trifluorometh- yl)-1H- pyrazole-4- carboxamide | ¹H NMR (400 MHz, DMSO- d₆) δ 1.36 (m, 2H), 1.57 (m, 2H), 1.83 (m, 4H), 2.10 (m, 1H), 3.23 (m, 4H), 3.40 (m, 1H), 3.90 (s, 3H), 7.53 (m, 2H), 7.63 (m, 1H), 7.86 (m, 2H), 7.97 (m, 2H), 8.29 (s, 1H), 8.35 (m, 1H), 8.69 (s, 1H); m/z (APCI pos) 517.1 (100%) [M + H]. |
| E182 | 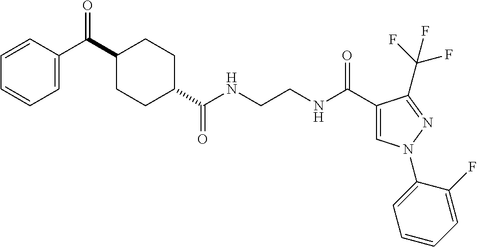 | N-(2-(trans- 4- benzoylcyclo- hexanecarbox- amido)ethyl)-1- (2- fluorophenyl)- 3- (trifluorometh- yl)-1H- pyrazole-4- carboxamide | ¹H NMR (400 MHz, DMSO- d₆) δ 1.35 (m, 2H), 1.56 (m, 2H), 1.83 (m, 4H), 2.11 (m, 1H), 3.22 (m, 2H), 3.28 (m, 2H), 3.40 (m, 1H), 7.35 (m, 1H), 7.53 (m, 2H), 7.61-7.71 (m, 4H), 7.87 (t, 1H, J = 5.5 Hz), 7.96 (m, 2H), 8.37 (t, 1H, J = 5.5 Hz), 9.09 (m, 1H); m/z (APCI pos) 531.1 (100%) [M + H]. |
| E184 | 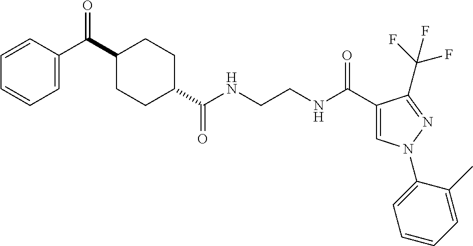 | N-(2-(trans- 4- benzoylcyclo- hexanecarbox- amido)ethyl)-1- o-tolyl-3- (trifluorometh- yl)-1H- pyrazole-4- carboxamide | ¹H NMR (400 MHz, DMSO- d₆) δ 1.35 (m, 2H), 1.56 (m, 2H), 1.82 (m, 4H), 2.10 (m, 1H), 2.22 (s, 3H), 3.19 (m, 2H), 3.27 (m, 2H), 3.38 (m, 1H), 7.39- 7.55 (m, 6H), 7.63 (m, 1H), 7.87 (m, 1H), 7.97 (m, 2H), 8.34 (m, 1H), 8.59 (s, 1H); m/z (APCI pos) 527.1 (100%) [M + H]. |
| E185 | 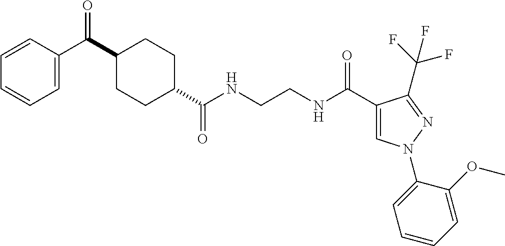 | N-(2-(trans- 4- benzoylcyclo- hexanecarbox- amido)ethyl)-1- (2- methoxyphenyl)- 3- (trifluorometh- yl)-1H- pyrazole-4- carboxamide | ¹H NMR (400 MHz, DMSO- d₆) δ 1.35 (m, 2H), 1.57 (m, 2H), 1.83 (m, 4H), 2.11 (m, 1H), 3.21 (m, 2H), 3.26 (m, 2H), 3.39 (m, 1H), 3.91 (s, 3H), 7.14 (m, 1H), 7.33 (m, 1H), 7.53 (m, 3H), 7.63 (m, 2H), 7.87 (t, 1H, J = 5.9 Hz), 7.97 (m, 2H), 8.38 (t, 1H, J = 5.5 Hz), 8.73 (s, 1H); m/z (APCI pos) 543.1 (100%) [M + H]. |
| E186 | 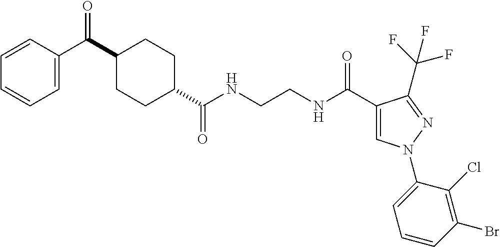 | N-(2-(trans- 4- benzoylcyclo- hexanecarbox- amido)ethyl)-1- (3-bromo-2- chlorophenyl)- 3- (trifluorometh- yl)-1H- pyrazole-4- carboxamide | ¹H NMR (400 MHz, DMSO- d₆) δ 1.35 (m, 2H), 1.57 (m, 2H), 1.83 (m, 4H), 2.10 (m, 1H), 3.21 (m, 2H), 3.26 (m, 2H), 3.39 (m, 1H), 7.53 (m, 3H), 7.63 (m, 1H), 7.74 (m, 1H), 7.86 (t, 1H, J = 5.5 Hz), 7.96 (m, 2H), 8.04 (m, 1H), 8.41 (t, 1H, J = 5.5 Hz), 8.71 (m, 1H); m/z (APCI pos) 627.0 (50%) [M + H]. |
| E187 | 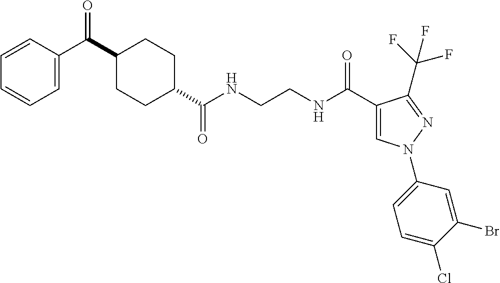 | N-(2-(trans- 4- benzoylcyclo- hexanecarbox- amido)ethyl)-1- (3-bromo-4- chlorophenyl)- 3- (trifluorometh- yl)-1H- pyrazole-4- carboxamide | ¹H NMR (400 MHz, DMSO- d₆) δ 1.36 (m, 2H), 1.58 (m, 2H), 1.84 (m, 4H), 2.11 (m, 1H), 3.21 (m, 2H), 3.28 (m, 2H), 3.40 (m, 1H), 7.53 (m, 2H), 7.63 (m, 1H), 7.88 (m, 3H), 7.96 (m, 2H), 8.23 (m, 1H), 8.35 (t, 1H, J = 5.5 Hz), 9.13 (m, 1H); m/z (APCI pos) 626.9 (50%) [M + H]. |

### Example E151

### N-(2-(trans-4-(3-Cyclopropyl-1,2,4-oxadiazol-5-yl)cyclohexanecarboxamido)ethyl)-1-phenyl-3-(trifluoromethyl)-1H-pyrazole-4-carboxamide

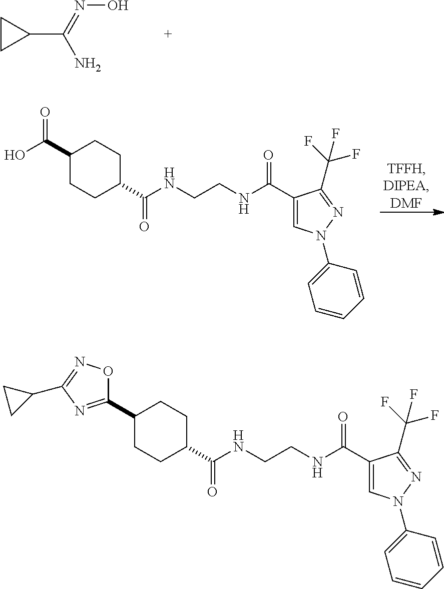

To trans-4-(2-(1-phenyl-3-(trifluoromethyl)-1H-pyrazole-4-carboxamido)ethylcarbamoyl)cyclohexanecarboxylic acid (100 mg, 0.21 mmol) in DMF (5 mL) were added DIPEA (32 mg, 0.24 mmol) and TFFH (64 mg, 0.24 mmol) at room temperature. The mixture was stirred at room temperature for 30 minutes then N′-hydroxycyclopropanecarboxamidine (24 mg, 0.24 mmol) was added at once. The mixture was stirred at 110° C. for 3 hours then concentrated to dryness. Purification of the residue by chromatography (DCM/MeOH 96/4) yielded a mixture of N-(2-(trans-4-(3-cyclopropyl-1,2,4-oxadiazol-5-yl)cyclohexanecarboxamido)ethyl)-1-phenyl-3-(trifluoromethyl)-1H-pyrazole-4-carboxamide and the reacted TFFH. This mixture was purified by recrystallization (AcOEt) to yield N-(2-(trans-4-(3-cyclopropyl-1,2,4-oxadiazol-5-yl)cyclohexanecarboxamido)ethyl)-1-phenyl-3-(trifluoromethyl)-1H-pyrazole-4-carboxamide as a white solid (89 mg, 78%): ¹H NMR (400 MHz, DMSO-d₆) δ 0.83-0.88 (m, 2H), 1.01-1.06 (m, 2H), 1.39-1.54 (m, 4H), 1.80-1.85 (m, 2H), 2.02-2.16 (m, 4H), 2.92 (m, 1H), 3.17-3.30 (m, 4H), 7.44-7.50 (m, 1H), 7.57-7.64 (m, 2H), 7.79-7.85 (m, 2H), 7.92 (t, 1H, J=5.5 Hz), 8.40 (t, 1H, J=5.5 Hz), 9.05 (s, 1H); m/z (APCI pos) 517.1 (100%) [M+H].

Compounds of the following structures were prepared from the corresponding hydroxylamidines, using a similar method to that described above.

| Ex # | Structure | Name | Physical Data |
|---|---|---|---|
| E152 | 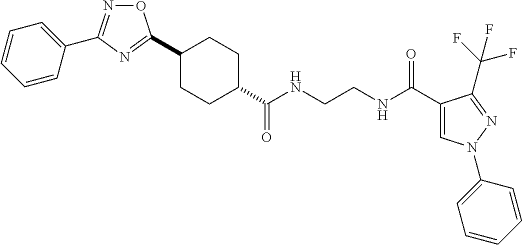 | 1-phenyl-N- (2-(trans-4- (3-phenyl- 1,2,4- oxadiazol-5- yl)cyclohexane- carboxamido) ethyl)-3- (trifluorometh- yl)-1H- pyrazole-4- carboxamide | White solid (63%); ¹H NMR (400 MHz, DMSO- d₆) δ 1.48-1.64 (m, 4H), 1.84-1.90 (m, 2H), 2.15-2.22 (m, 3H), 3.08 (m, 1H), 3.17- 3.32 (m, 4H), 7.45- 7.65 (m, 6H), 7.80- 7.84 (m, 2H), 7.92 (t, 1H, J = 5.5 Hz), 7.98- 8.03 (m, 2H), 8.38 (t, 1H, J = 5.5 Hz), 9.05 (s, 1H); m/z (APCI pos) 553.1 (100%) [M + H]. |
| E153 | 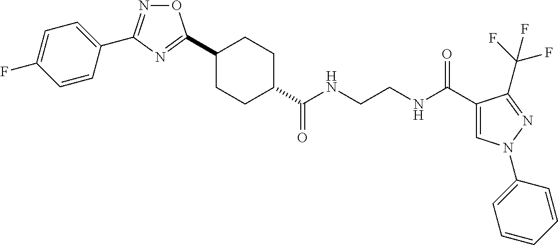 | N-(2-(trans- 4-(3-(4- fluorophenyl)- 1,2,4- oxadiazol-5- yl)cyclohexane- carboxamido) ethyl)-1- phenyl-3- (trifluorometh- yl)-1H- pyrazole-4- carboxamide | White solid (49%); ¹H NMR (400 MHz, DMSO- d₆) δ 1.48-1.64 (m, 4H), 1.84-1.90 (m, 2H), 2.15-2.22 (m, 3H), 3.08 (m, 1H), 3.17- 3.32 (m, 4H), 7.37- 7.43 (m, 2H), 7.46- 7.51 (m, 1H), 7.58- 7.64 (m, 2H), 7.80- 7.83 (m, 2H), 7.91 (t, 1H, J = 5.5 Hz), 8.03- 8.08 (m, 2H), 8.37 (t, 1H, J = 5.5 Hz), 9.05 (s, 1H); m/z (APCI pos) 571.1 (100%) [M + H]. |
| E154 | 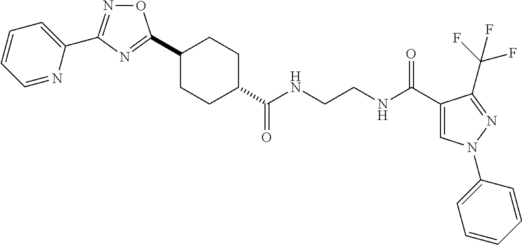 | 1-phenyl-N- (2-(trans-4- (3-(pyridin- 2-yl)-1,2,4- oxadiazol-5- yl)cyclohexane- carboxamido) ethyl)-3- (trifluorometh- yl)-1H- pyrazole-4- carboxamide | White solid (61%); ¹H NMR (400 MHz, DMSO- d₆) δ 1.48-1.64 (m, 4H), 1.84-1.90 (m, 2H), 2.15-2.22 (m, 3H), 3.11 (m, 1H), 3.17- 3.32 (m, 4H), 7.46- 7.51 (m, 1H), 7.58- 7.64 (m, 3H), 7.80- 7.83 (m, 2H), 7.95 (t, 1H, J = 5.5 Hz), 7.99- 8.08 (m, 2H), 8.41 (t, 1H, J = 5.5 Hz), 8.74- 8.77 (m, 1H), 9.06 (s, 1H); m/z (APCI pos) 554.2 (100%) [M + H]. |
| E155 | 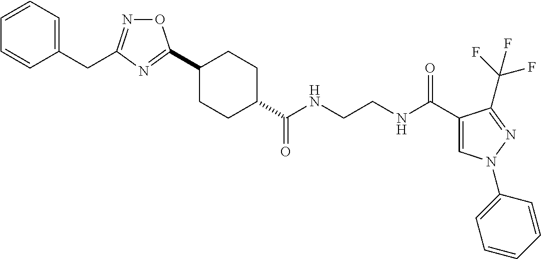 | N-(2-(trans- 4-(3-benzyl- 1,2,4- oxadiazol-5- yl)cyclohexane- carboxamido) ethyl)-1- phenyl-3- (trifluorometh- yl)-1H- pyrazole-4- carboxamide | White solid (28%); ¹H NMR (400 MHz, DMSO- d₆) δ 1.40-1.54 (m, 4H), 1.80-1.85 (m, 2H), 2.04-2.17 (m, 3H), 2.96 (m, 1H), 3.17- 3.30 (m, 4H), 4.05 (s, 2H), 7.23-7.35 (m, 5H), 7.45-7.50 (m, 1H), 7.58-7.64 (m, 2H), 7.80-7.83 (m, 2H), 7.87 (t, 1H, J = 5.5 Hz), 8.36 (t, 1H, J = 5.5 Hz), 9.03 (s, 1H); m/z (APCI pos) 567.1 (100%) [M + H]. |

### Example E167

### N1-Phenyl-N4-(2-(1-phenyl-3-(trifluoromethyl)-1H-pyrazole-4-carboxamido)ethyl)piperidine-1,4-dicarboxamide

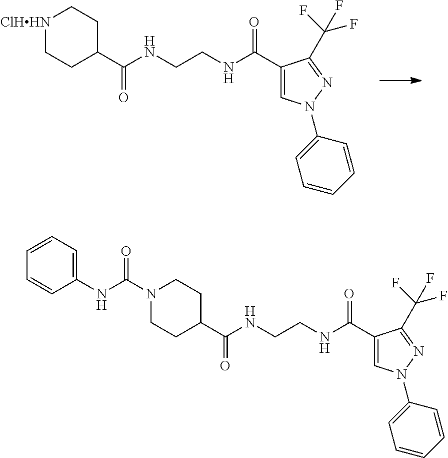

To a solution of N-(2-(1-phenyl-3-(trifluoromethyl)-1H-pyrazole-4-carboxamido)ethyl)piperidine-4-carboxamide hydrochloride (0.070 g, 0.1570 mmol) and DIPEA (0.05469 mL, 0.3140 mmol) in DMA (5 mL) was added phenylisocyanate (0.01706 mL, 0.1570 mmol). The mixture was stirred for 3 hours. The solvent was removed in vacuo and the residue was dissolved in AcOEt and left to a precipitate. The precipitate was collected by filtration and dried to give N1-phenyl-N4-(2-(1-phenyl-3-(trifluoromethyl)-1H-pyrazole-4-carboxamido)ethyl)piperidine-1,4-dicarboxamide (0.063 g, 76% yield) as a solid: ¹H NMR (400 MHz, CD₃OD) δ 1.47 (m, 2H), 1.69 (m, 2H), 2.32 (m, 1H), 2.78 (m, 2H), 3.22 (m, 2H), 3.28 (m, 2H), 4.11 (m, 2H), 6.91 (m, 1H), 7.21 (m, 2H), 7.46 (m, 3H), 7.61 (m, 2H), 7.81 (m, 2H), 7.93 (m, 1H), 8.37 (m, 1H), 8.45 (s, 1H), 9.04 (s, 1H); m/z (APCI pos) 529.1 (100%) [M+11].

### Example E172

### 1-Phenyl-N-(2-(trans-4-(4-phenyl-1H-imidazol-2-yl)cyclohexanecarboxamido)ethyl)-3-(trifluoromethyl)-1H-pyrazole-4-carboxamide

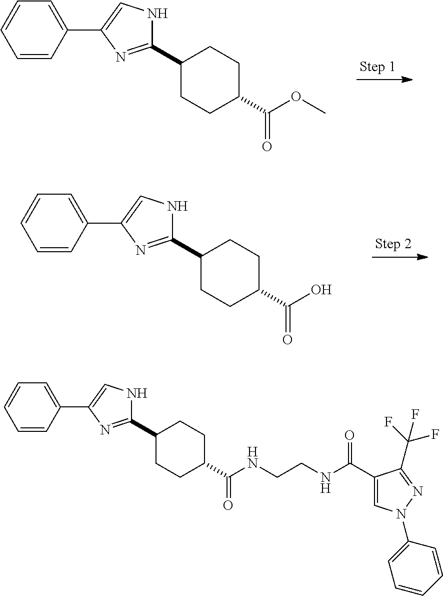

### Step 1

To trans-methyl 4-(4-phenyl-1H-imidazol-2-yl)cyclohexanecarboxylate (1.0 g, 3.5 mmol) in THF:H₂O (1:1, 100 mL) was added lithium hydroxide monohydrate (590 mg, 14 mmol) and the mixture was stirred at room temperature for 18 hours. After concentration, the residue was diluted with water and acidified to pH 5-6. The aqueous layer was extracted with EtOAc (4×100 mL) and the combined organic layers were washed with brine and dried over MgSO₄ to yield trans-4-(4-phenyl-1H-imidazol-2-yl)cyclohexanecarboxylic acid as a white solid (610 mg, 64%): ¹H NMR (400 MHz, DMSO-d₆) δ 1.30-1.60 (m, 4H), 1.90-2.05 (m, 4H), 2.20-2.28 (m, 1H), 2.60-2.69 (m, 1H), 7.12-7.17 (m, 1H), 7.29-7.34 (m, 2H), 7.40 (s, 1H), 7.68-7.72 (m, 2H): m/z (APCI pos) 271.1 (100%) [M+H].

### Step 2

To trans-4-(4-phenyl-1H-imidazol-2-yl)cyclohexanecarboxylic acid (136 mg, 0.5 mmol) in DCM (50 mL) were successively added N-(2-aminoethyl)-1-phenyl-3-(trifluoromethyl)-1H-pyrazole-4-carboxamide (150 mg, 0.5 mmol), HOBt.H₂O (95 mg, 0.7 mmol), EDAC.HCl (125 mg, 0.65 mmol) and triethylamine (102 mg, 1.0 mmol). The mixture was stirred at room temperature for 18 hours then DCM was added (50 mL), and the organic layer was successively washed with 2N HCl, 10% aqueous K₂CO₃ and brine. After drying over MgSO₄ and concentration, the residue was purified by MPLC (DCM/MeOH 95/5). Crystallization from AcOEt/MeOH yielded a white solid which proved to be an impurity. A second crystallization in the same conditions completely removed the impurity. Final crystallization from AcOEt/hexane yielded 1-phenyl-N-(2-(trans-4-(4-phenyl-1H-imidazol-2-yl)cyclohexanecarboxamido) ethyl)-3-(trifluoromethyl)-1H-pyrazole-4-carboxamide as a white solid (89 mg, 32%): ¹H NMR (400 MHz, DMSO-d₆) δ 1.42-1.55 (m, 4H), 1.80-2.05 (m, 4H), 2.10-2.18 (m, 1H), 2.60-2.66 (m, 1H), 3.15-3.32 (m, 4H), 7.12-7.17 (m, 1H), 7.29-7.34 (m, 2H), 7.40 (br, 1H), 7.45-7.50 (m, 1H), 7.59-7.64 (m, 2H), 7.68-7.72 (m, 2H), 7.79-7.84 (m, 2H), 7.89 (t, 1H, J=5.9 Hz), 8.38 (t, 1H, J=5.5 Hz), 9.06 (s, 1H), 11.80 (br, 1H); m/z (APCI pos) 551.3 (100%) [M+H].

### Example E180

### N-(2-(1-Benzoylpyrrolidine-3-carboxamido)ethyl)-1-phenyl-3-(trifluoromethyl)-1H-pyrazole-4-carboxamide

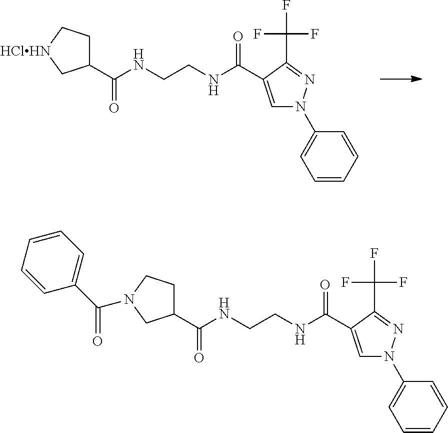

Benzoic acid (0.03111 g, 0.2547 mmol), HOBt.H₂O (0.04381 g, 0.3242 mmol), EDAC.HCl (0.05771 g, 0.3010 mmol) and triethylamine (0.06455 mL, 0.4631 mmol) were combined in DCM (20 mL) and the mixture was stirred for 30 minutes at room temperature, then 1-phenyl-N-(2-(pyrrolidine-3-carboxamido)ethyl)-3-(trifluoromethyl)-1H-pyrazole-4-carboxamide hydrochloride (0.100 g, 0.2316 mmol) was added. The mixture was stirred at room temperature for 18 hours then diluted with DCM, washed with 2N HCl, 10% wt aqueous K₂CO₃ and brine and dried over Na₂SO₄. Concentration yielded a residue which was crystallized from AcOEt/hexanes to give N-(2-(1-benzoylpyrrolidine-3-carboxamido)ethyl)-1-phenyl-3-(trifluoromethyl)-1H-pyrazole-4-carboxamide (0.090 g, 78%) as white a solid: ¹H NMR (400 MHz, DMSO-d₆) δ 1.89-2.10 (m, 2H), 2.87-3.10 (m, 1H), 3.18-3.30 (m, 4H), 3.41-3.71 (m, 4H), 7.40-7.50 (m, 6H), 7.61 (t, J=7.9 Hz, 2H), 7.81 (t, J=6.8 Hz, 2H), 8.05-8.16 (m, 1H), 8.34-8.40 (m, 1H), 9.00 (s, 0.5H), 9.04 (s, 0.5H); m/z (APCI pos) 500 [M+H].

The compound of the following structure was prepared from the corresponding acid, using a similar method to that described above

| Ex # | Structure | Name | Physical Data |
|---|---|---|---|
| E181 | 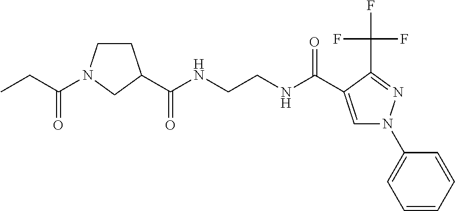 | 1-phenyl-N- (2-(1- propionyl- pyrrolidine-4- carboxamido) ethyl)-3- (trifluorometh- yl)-1H- pyrazole-4- carboxamide | ¹H NMR (400 MHz, CDCl₃) δ 1.12 (t, J = 7.4 Hz, 3H), 1.26-2.33 (m, 4H), 2.83-3.01 (m, 1H), 3.38-3.80 (m, 8H), 6.48-6.70 (m, 2H), 7.39-7.45 (m, 1H), 7.49-7.54 (m, 2H), 7.71-7.77 (m, 2H), 8.45 (s, 0.5 H), 8.55 (s, 0.5 H); m/z (APCI pos) MS Calcd.: 451; Found: 452 (M + H). (100%) [M + H]. |

### Example E183

### 2-Methyl-N-(2-(1-phenyl-3-(trifluoromethyl)-1H-pyrazole-4-carboxamido)ethyl)-1-(pyrrolidine-1-carbonyl)piperidine-4-carboxamide

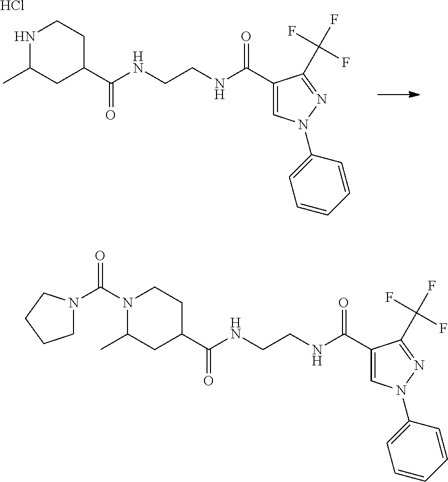

A mixture of 2-methyl-N-(2-(1-phenyl-3-(trifluoromethyl)-1H-pyrazole-4-carboxamido)ethyl)piperidine-4-carboxamide hydrochloride (0.20 g, 0.44 mmol), pyrrolidine-1-carbonyl chloride (0.064 g, 0.48 mmol), N,N′-diisopropylethylamine (0.17 mL, 0.96 mmol) and DMF (3 mL) was heated at 70° C. for 24 hours. After cooling, the mixture was diluted with AcOEt, washed with water and brine, dried and concentrated in vacuo. The residue was purified by chromatography on silica (AcOEt->AcOEt/MeOH 10/1->AcOEt/MeOH/NH₄OH 20/2/1) to give 2-methyl-N-(2-(1-phenyl-3-(trifluoromethyl)-1H-pyrazole-4-carboxamido)ethyl)-1-(pyrrolidine-1-carbonyl)piperidine-4-carboxamide (0.035 g, 16%) as colorless crystals: ¹H NMR (400 MHz, CDCl₃) δ 1.14 (3H, d, J=6.4 Hz), 1.56 (1H, m), 1.71 (1H, m), 1.76-1.90 (6H, m), 2.28 (1H, m), 2.84 (1H, m), 3.14-3.28 (2H, m), 3.28-3.44 (4H, m), 3.36-3.54 (2H, m), 3.56-3.64 (2H, m), 6.35 (1H, t, J=5.2 Hz), 6.85 (1H, br), 7.40 (1H, t, J=7.6 Hz), 7.51 (2H, t, J=7.6 Hz), 7.72 (2H, d, J=7.6 Hz), 8.43 (1H, s); m/z (APCI pos) 521.1 (100%).

### Example E196

### 2-Isopropyl-N-(2-(1-phenyl-3-(trifluoromethyl)-1H-pyrazole-4-carboxamido)ethyl)-5,6,7,8-tetrahydroquinazoline-6-carboxamide

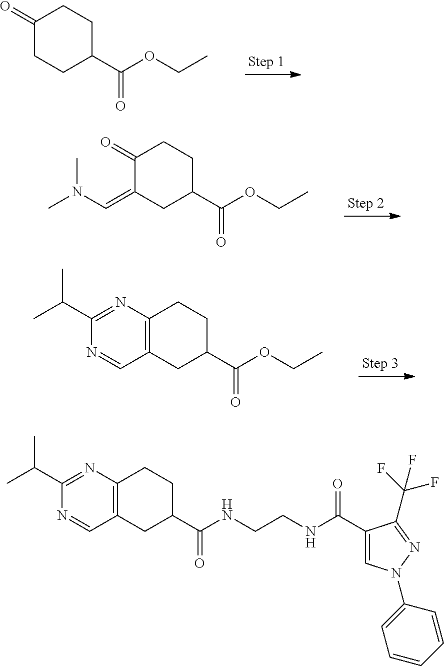

### Step 1

Ethyl 4-oxocyclohexanecarboxylate (4.0 g, 24 mmol), dimethoxy-N,N-dimethylmethanamine (2.8 g; 24 mmol) and triethylamine (4.8 g, 48 mmol) were heated to 100° C. in a sealed tube for 18 hours. After cooling down, the mixture was concentrated and purified by MPLC (AcOEt/hexanes 50/50->100/0) to give ethyl 3-((dimethylamino)methylene)-4-oxocyclohexanecarboxylate as a clear oil (3.8 g, 72%): ¹H NMR (400 MHz, CDCl₃) δ 1.28 (t, 3H, J=7.0 Hz), 1.80-1.92 (m, 1H), 2.08-2.16 (m, 1H), 2.30-2.62 (m, 3H), 2.77-2.88 (m, 1H), 2.98-3.05 (m, 1H), 3.11 (s, 6H), 4.17 (q, 2H, J=7.0 Hz), 7.53 (s, 1H).

### Step 2

To ethyl 3-((dimethylamino)methylene)-4-oxocyclohexanecarboxylate (700 mg, 3.1 mmol) in EtOH (10 mL) was added isobutyramidine hydrochloride (1.9 g, 15.5 mmol) and the mixture was heated to reflux for 18 hours. After cooling down, the mixture was concentrated to dryness and water (30 mL) was added. The aqueous layer was extracted with DCM (3×30 mL) and the combined organic layers were washed with brine and dried over MgSO₄. Concentration yielded ethyl 2-isopropyl-5,6,7,8-tetrahydroquinazoline-6-carboxylate as a yellow oil (750 mg, 98%): ¹H NMR (400 MHz, CDCl₃) δ 1.29 (t, 3H, J=7.0 Hz), 1.31 (s, 3H), 1.33 (s, 3H), 1.90-2.01 (m, 1H), 2.25-3.00 (m, 6H), 3.10-3.17 (m, 1H), 4.19 (q, 2H, J=7 Hz), 8.39 (s, 1H); m/z (APCI pos) 249.2 (100%) [M+H].

### Step 3

To ethyl 2-isopropyl-5,6,7,8-tetrahydroquinazoline-6-carboxylate (300 mg, 1 mmol) in MeOH (30 mL) was added 10% NaOH (4 mL) and the mixture was heated to reflux for 2 hours. After cooling down, the mixture was concentrated to dryness to yield the corresponding sodium carboxylate. To sodium 2-isopropyl-5,6,7,8-tetrahydroquinazoline-6-carboxylate (650 mg, 2.7 mmol) in DCM (100 mL)/DMF (20 mL) were successively added N-(2-aminoethyl)-1-phenyl-3-(trifluoromethyl)-1H-pyrazole-4-carboxamide (800 mg, 2.7 mmol), EDAC.HCl (669 mg, 3.5 mmol), HOBt.H₂O (508 mg, 3.8 mmol) and triethylamine (543 mg, 5.37 mmol). The mixture was stirred at room temperature for 18 hours then concentrated to dryness. DCM was added (100 mL). The organic layer was successively washed with 0.1N HCl (50 mL), 10% potassium carbonate aqueous solution (50 mL) and brine (2×50 mL). The organic layer was dried over MgSO₄ then concentrated to yield a yellow solid which was purified by reverse phase HPLC (Paraflex Biotage system. XTerra prep RP18 OBD 30×150 mm column. Method 20 min 5-95% water/ACN) to yield 2-isopropyl-N-(2-(1-phenyl-3-(trifluoromethyl)-1H-pyrazole-4-carboxamido)ethyl)-5,6,7,8-tetrahydroquinazoline-6-carboxamide as a white solid (359 mg, 27%): ¹H NMR (400 MHz, DMSO-d₆) δ 1.16 (s, 3H), 1.18 (s, 3H), 1.69-1.80 (m, 1H), 1.94-2.01 (m, 1H), 2.45-2.55 (m, 1H), 2.69-2.76 (m, 4H), 2.92-3.00 (m, 1H), 3.15-3.30 (m, 4H), 7.40-7.45 (m, 1H), 7.52-7.59 (m, 2H), 7.73-7.78 (m, 2H), 8.03 (t, 1H, J=5.5 Hz), 8.33 (s, 1H), 8.34 (t, 1H, J=5.5 Hz), 8.99 (s, 1H); m/z (APCI pos) 501.4 (100%) [M+H].

### Example E200

### N-(2-(trans-4-(1-Methyl-4-phenyl-1H-imidazol-2-yl)cyclohexanecarboxamido)ethyl)-1-phenyl-3-(trifluoromethyl)-1H-pyrazole-4-carboxamide

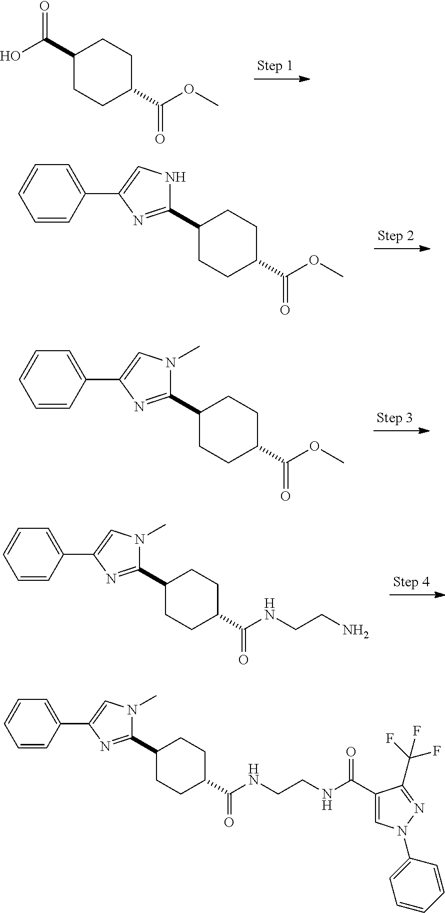

### Step 1

To trans-4-(methoxycarbonyl)cyclohexanecarboxylic acid (1.0 g, 5.4 mmol) in DCM (100 mL) were successively added 2-amino-1-phenylethanone hydrochloride (0.92 g, 5.4 mmol), HOBt.H₂O (1.0 g, 7.5 mmol), EDAC.HCl (1.3 g, 7.0 mmol) and triethylamine (1.6 g, 16 mmol). The mixture was stirred at room temperature for 18 hours then DCM was added (100 mL) and the organic layer was successively washed with 2N HCl, 10% aqueous K₂CO₃ and brine. After drying over MgSO₄ and concentration, the resulting tan solid (1.4 g, 86%) was dissolved in AcOH (20 mL). Ammonium acetate (3.3 g, 46 mmol) was added and the mixture was heated to reflux for 20 hours. After concentration, the residue was partitioned between 10% NaOH and AcOEt (1:1) and the organic layer was washed 3 times with 10% NaOH and brine. After drying on MgSO₄ and concentration, trans-methyl 4-(4-phenyl-1H-imidazol-2-yl)cyclohexanecarboxylate was isolated as a brown solid (900 mg, 69%): ¹H NMR (400 MHz, CDCl₃) δ 1.50-1.64 (m, 4H), 2.04-2.20 (m, 4H), 2.30-2.40 (m, 1H), 2.70-2.80 (m, 1H), 3.68 (s, 3H), 7.18-7.25 (m, 2H), 7.32-7.38 (m, 2H), 7.48-7.54 (m, 1H), 7.64-7.70 (m, 1H); m/z (APCI pos) 285.3 (100%) [M+H].

### Step 2

To trans-methyl 4-(4-phenyl-1H-imidazol-2-yl)cyclohexanecarboxylate (900 mg, 3.2 mmol) in acetone (50 mL) was added potassium carbonate (480 mg, 3.5 mmol). The mixture was heated to 50° C. then dimethyl sulfate (400 mg, 3.2 mmol) was added at once. The mixture was stirred at 50° C. for 20 hours then concentrated and purified by MPLC (DCM/MeOH 98/2) to yield trans-methyl 4-(1-methyl-4-phenyl-1H-imidazol-2-yl)cyclohexanecarboxylate as an orange solid (510 mg, 56%): ¹H NMR (400 MHz, DMSO-d₆) δ 1.35-1.66 (m, 4H), 1.87-2.03 (m, 4H), 2.35-2.45 (m, 1H), 2.70-2.79 (m, 1H), 3.61 (s, 3H), 3.62 (s, 3H), 7.11-7.16 (m, 1H), 7.27-7.33 (m, 2H), 7.45 (s, 1H), 7.66-7.70 (m, 2H); m/z (APCI pos) 299.2 (100%) [M+H].

### Step 3

trans-Methyl 4-(1-methyl-4-phenyl-1H-imidazol-2-yl)cyclohexanecarboxylate (500 mg, 1.68 mmol) was heated to reflux in ethylenediamine (10 mL) for 3 hours. The mixture was concentrated to dryness then toluene was added, and the solution concentrated to dryness. The process was repeated 2 more times before leaving trans-N-(2-aminoethyl)-4-(1-methyl-4-phenyl-1H-imidazol-2-yl)cyclohexanecarboxamide as a brown solid (540 mg, quant): ¹H NMR (400 MHz, DMSO-d₆) δ 1.50-1.60 (m, 4H), 1.79-1.92 (m, 4H), 2.52-2.60 (m, 1H), 2.69-2.75 (m, 1H), 3.00-3.30 (m, 4H), 3.61 (s, 3H), 7.10-7.20 (m, 1H), 7.28-7.32 (m, 2H), 7.44 (s, 1H), 7.68-7.70 (m, 2H), 7.75 (t, 1H, J=5.5 Hz); m/z (APCI pos) 327.2 (100%) [M+H].

### Step 4

To 1-phenyl-3-(trifluoromethyl)-1H-pyrazole-4-carboxylic acid (275 mg, 1.07 mmol) in DCM (100 mL)/DMF (20 mL) were successively added trans-N-(2-aminoethyl)-4-(1-methyl-4-phenyl-1H-imidazol-2-yl)cyclohexanecarboxamide (267 mg, 1.07 mmol), EDAC.HCl (267 mg, 1.4 mmol), HOBt.H₂O (203 mg, 1.5 mmol) and triethylamine (217 mg, 2.1 mmol). The mixture was stirred at room temperature for 18 hours then it was concentrated to dryness. DCM was added (100 mL). The organic layer was successively washed with 0.1N HCl (50 mL), 10% potassium carbonate aqueous solution (50 mL) and brine (2×50 mL). The organic layer was dried over MgSO₄ then concentrated and purified by reverse phase HPLC (Paraflex Biotage system. XTerra prep RP18 OBD 30×150 mm column. Method 20 min 5-95% water/CH₃CN) to yield N-(2-(trans-4-(1-methyl-4-phenyl-1H-imidazol-2-yl)cyclohexanecarboxamido)ethyl)-1-phenyl-3-(trifluoromethyl)-1H-pyrazole-4-carboxamide as a white solid (173 mg, 29%): ¹H NMR (400 MHz, DMSO-d₆) δ 1.50-1.60 (m, 4H), 1.80-1.93 (m, 4H), 2.14-2.23 (m, 1H), 2.68-2.76 (m, 1H), 3.20-3.35 (m, 4H), 3.60 (s, 3H), 7.12-7.17 (m, 1H), 7.28-7.33 (m, 2H), 7.44 (s, 1H), 7.45-7.50 (m, 1H), 7.58-7.64 (m, 2H), 7.66-7.70 (m, 2H), 7.80-7.84 (m, 2H), 7.89 (t, 1H, J=5.5 Hz), 8.38 (t, 1H, J=5.5 Hz), 9.05 (s, 1H); m/z (APCI pos) 565.3 (100%) [M+H].

### Example E202 and E203

### N-(2-(trans-4-(5-(Chloromethyl)-1,3,4-oxadiazol-2-yl)cyclohexanecarboxamido)ethyl)-1-phenyl-3-(trifluoromethyl)-1H-pyrazole-4-carboxamide and N-(2-(trans-4-(5-(methoxymethyl)-1,3,4-oxadiazol-2-yl)cyclohexanecarboxamido)ethyl)-1-phenyl-3-(trifluoromethyl)-1H-pyrazole-4-carboxamide

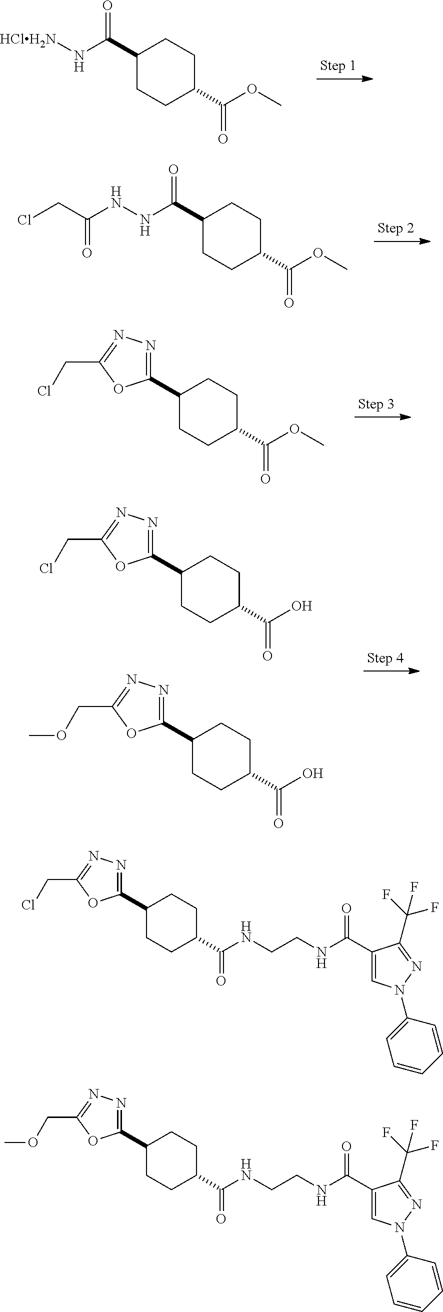

### Step 1

To a solution of N-methylmorpholine (0.5109 mL, 4.647 mmol) and trans-methyl 4-(hydrazinocarbonyl)cyclohexanecarboxylate hydrochloride (0.500 g, 2.112 mmol) in DCM (10 mL) at 0-5° C., was added a solution of 2-chloroacetyl chloride (0.1681 mL, 2.112 mmol) in DCM (5 mL). The mixture was stirred at room temperature for 18 hours then it was partitioned between a saturated aqueous NaHCO₃ and 25% iPrOH/DCM. The organic layer was dried over Na₂SO₄, filtered and concentrated to give trans-methyl 4-(N′-(2-chloroacetyl)hydrazinocarbonyl)cyclohexanecarboxylate (0.408 g, 70%): m/e (APCI neg) 275 [M−H].

### Step 2

A mixture of trans-methyl 4-(N′-(2-chloroacetyl)hydrazinocarbonyl)cyclohexanecarboxylate (0.300 g, 1.08 mmol) and POCl₃ (0.397 mL, 4.34 mmol) in CH₃CN (20 mL) was stirred at 80° C. for 16 hours. The mixture was concentrated to a residue which was dissolved in DCM, and washed with a saturated aqueous NaHCO₃ and then water. The organic layer was dried over Na₂SO₄ and concentrated to give trans-methyl 4-(5-(chloromethyl)-1,3,4-oxadiazol-2-yl)cyclohexanecarboxylate (0.286 g, quant.): ¹H NMR (400 MHz, DMSO-d₆) δ 1.53-1.71 (m, 4H), 2.11-2.17 (m, 2H), 2.24-2.27 (m, 2H), 2.35-2.42 (m, 1H), 2.88-2.94 (m, 1H), 3.70 (s, 3H), 4.68 (s, 2H).

### Step 3

A mixture of trans-methyl 4-(5-(chloromethyl)-1,3,4-oxadiazol-2-yl)cyclohexane carboxylate (0.080 g, 0.309 mmol) and 2N NaOH (0.618 mL, 0.618 mmol) in MeOH (20 mL) was stirred at 50° C. for 4 hours. The mixture was concentrated to dryness and the residue diluted with water and washed with DCM. The aqueous layer was made acidic with 1N HCl and was extracted with AcOEt. The combined organic layers were washed with water, dried over Na₂SO₄ and concentrated to give a mixture of trans-4-(5-(chloromethyl)-1,3,4-oxadiazol-2-yl)cyclohexanecarboxylic acid (minor) and trans-4-(5-(methoxymethyl)-1,3,4-oxadiazol-2-yl)cyclohexanecarboxylic acid (major) (56 mg total).

### Step 4

To the previously isolated mixture of trans-4-(5-(chloromethyl)-1,3,4-oxadiazol-2-yl)cyclohexanecarboxylic acid (minor) and trans-4-(5-(methoxymethyl)-1,3,4-oxadiazol-2-yl)cyclohexanecarboxylic acid (major) (56 mg total) in CH₃CN (20 mL), were added N-(2-aminoethyl)-1-phenyl-3-(trifluoromethyl)-1H-pyrazole-4-carboxamide (0.0751 g, 0.252 mmol), EDAC.HCl (0.0658 g, 0.343 mmol) and HOBt.H₂O (0.0526 g, 0.343 mmol). The mixture was stirred at room temperature for 18 hours then it was concentrated to dryness and dissolved in DCM (50 mL). The organic layer was washed with 1N HCl and saturated aqueous NaHCO₃, dried over Na₂SO₄ and concentrated to a residue. The residue was purified by reverse phase HPLC to give 2 compounds.

N-(2-(trans-4-(5-(chloromethyl)-1,3,4-oxadiazol-2-yl)cyclohexanecarboxamido)ethyl)-1-phenyl-3-(trifluoromethyl)-1H-pyrazole-4-carboxamide (0.010 g, 8.32% yield) as a white solid: ¹H NMR (400 MHz, CDCl₃) δ 1.57-1.70 (m, 4H), 2.01-2.06 (m, 2H), 2.18-2.27 (m, 3H), 2.91 (br, 1H), 3.50-3.54 (m, 2H), 3.60-3.64 (m, 2H), 4.67 (s, 2H), 6.24 (br, 1H), 6.69 (br, 1H), 7.43 (t, J=7.4 Hz, 1H), 7.52 (t, J=7.9 Hz, 2H), 7.72 (d, J=7.4 Hz, 2H), 8.41 (s, 1H); m/e (APCI pos) 525 [M+H].

N-(2-(trans-4-(5-(methoxymethyl)-1,3,4-oxadiazol-2-yl)cyclohexane carboxamido)ethyl)-1-phenyl-3-(trifluoromethyl)-1H-pyrazole-4-carboxamide (0.058 g, 48.7% yield) as a white solid: ¹H NMR (400 MHz, CDCl₃) δ 1.55-1.70 (m, 4H), 2.02-2.05 (m, 2H), 2.17-2.26 (m, 3H), 2.87-2.92 (m, 1H), 3.45 (s, 3H), 3.47-3.53 (m, 2H), 3.59-3.63 (m, 2H), 4.61 (s, 2H), 6.35 (t, J=5.1 Hz, 1H), 6.81 (br, 1H), 7.42 (t, J=7.3 Hz, 1H), 7.52 (t, J=7.8 Hz, 2H), 7.71 (d, J=7.4 Hz, 2H), 8.42 (s, 1H). m/e (APCI pos) 521 [M+H].

### Example E213

### Methyl 2-(5-(trans-4-(2-(1-phenyl-3-(trifluoromethyl)-1H-pyrazole-4-carboxamido)ethylcarbamoyl)cyclohexyl)-1,3,4-oxadiazol-2-yl)acetate

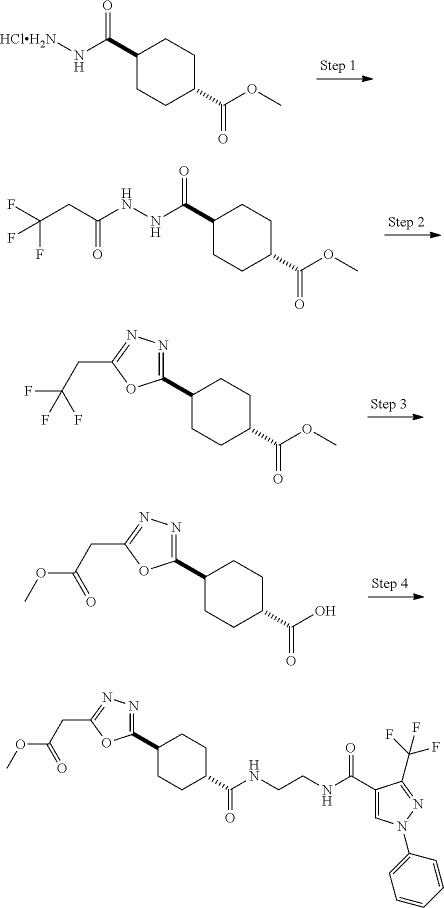

### Step 1

A mixture of 3,3,3-trifluoropropanoic acid (0.08208 mL, 0.9295 mmol), EDAC.HCl (0.2430 g, 1.267 mmol) and HOBt.H₂O (0.1941 g, 1.267 mmol) in CH₃CN was stirred for 30 minutes at room temperature, then triethylamine (0.1531 mL, 1.098 mmol) and trans-methyl 4-(hydrazinocarbonyl)cyclohexanecarboxylate hydrochloride (0.200 g, 0.8450 mmol) were added. The mixture was stirred at room temperature for 18 hours then it was concentrated to dryness and dissolved in 25% iPrOH/DCM. The organic layer was washed with 1N HCl, saturated aqueous NaHCO₃, dried over Na₂SO₄ and concentrated to a residue. The residue was purified by chromatography on silica (5% MeOH/DCM) to give trans-methyl 4-(N′-(3,3,3-trifluoropropanoyl)hydrazinocarbonyl)cyclohexanecarboxylate (0.220 g, 84%) as a tan solid: ¹H NMR (400 MHz, DMSO-d₆) δ 1.27-1.46 (m, 4H), 1.77 (d, J=12.9 Hz, 2H), 1.94 (d, J=12.5 Hz, 2H), 2.12-2.20 (m, 1H), 2.26-2.33 (m, 1H), 3.22-3.39 (m, 2H), 3.59 (s, 3H), 10.18 (s, 1H), 10.18 (s, 1H).

### Step 2

A mixture of trans-methyl 4-(N′-(3,3,3-trifluoropropanoyl)hydrazinocarbonyl)cyclohexanecarboxylate (0.200 g, 0.645 mmol) in POCl₃ (20 mL, 218 mmol) was heated to 100° C. for 18 hours. The mixture was then concentrated to a residue which was dissolved in DCM. The organic layer was washed with saturated aqueous NaHCO₃, dried over Na₂SO₄ and concentrated to a residue. This residue was purified by chromatography on silica (40% AcOEt/hexanes then 5% MeOH/DCM) to give trans-methyl 4-(5-(2,2,2-trifluoroethyl)-1,3,4-oxadiazol-2-yl)cyclohexanecarboxylate (0.095 g, 50%): ¹H NMR (400 MHz, CDCl₃) δ 1.53-1.70 (m, 4H), 2.15 (d, J=11.1 Hz, 2H), 2.24 (d, J=10.7 Hz, 2H), 2.35-2.42 (m, 1H), 2.87-2.94 (m, 1H), 3.69-3.88 (m, 5H).

### Step 3

A mixture of trans-methyl 4-(5-(2,2,2-trifluoroethyl)-1,3,4-oxadiazol-2-yl)cyclohexanecarboxylate (0.090 g, 0.308 mmol) and 2N NaOH (0.616 mL, 0.616 mmol) in MeOH (10 mL) was stirred at 50° C. for 4 hours. The mixture was concentrated to dryness and diluted with water then washed with DCM. The aqueous layer was made acidic with 1N HCl and extracted with AcOEt. The combined organic layers were washed with water, dried over Na₂SO₄ and concentrated to give trans-4-(5-(2-methoxy-2-oxoethyl)-1,3,4-oxadiazol-2-yl)cyclohexanecarboxylic acid (0.040 g, 51% yield).

### Step 4

A mixture of N-(2-aminoethyl)-1-phenyl-3-(trifluoromethyl)-1H-pyrazole-4-carboxamide (0.0472 g, 0.158 mmol), EDAC.HCl (0.0413 g, 0.216 mmol), HOBt.H₂O (0.0330 g, 0.216 mmol) and trans-4-(5-(2-methoxy-2-oxoethyl)-1,3,4-oxadiazol-2-yl)cyclohexanecarboxylic acid (0.040 g, 0.144 mmol) in CH₃CN (25 mL) was stirred at room temperature for 18 hours. The mixture was concentrated to a residue, the residue was diluted with AcOEt, and the mixture was washed with 1N HCl and saturated aqueous NaHCO₃, dried over Na₂SO₄ and concentrated to a residue. The residue was purified by reverse phase HPLC to give methyl 2-(5-(trans-4-((2-(1-phenyl-3-(trifluoromethyl)-1H-pyrazole-4-carboxamido)ethyl)carbamoyl)cyclohexyl)-1,3,4-oxadiazol-2-yl)acetate (0.055 g, 69.7% yield) as a white solid: ¹H NMR (400 MHz, CDCl₃) δ 1.55-1.69 (m, 4H), 2.01-2.04 (m, 2H), 2.18-2.26 (m, 3H), 2.88-2.90 (m, 1H), 3.49-3.53 (m, 2H), 3.59-3.63 (m, 2H), 3.77 (s, 3H), 3.93 (s, 2H), 6.31 (br, 1H), 6.78 (br, 1H), 7.43 (t, J=7.4 Hz, 1H), 7.52 (t, J=7.8 Hz, 2H), 7.71 (d, J=7.6 Hz, 2H), 8.42 (s, 1H); m/e (APCI pos) 549 [M+11].

### Example E218

### N-(2-(trans-4-(Hydroxy(phenyl)methyl)cyclohexanecarboxamido)ethyl)-1-phenyl-3-(trifluoromethyl)-1H-pyrazole-4-carboxamide

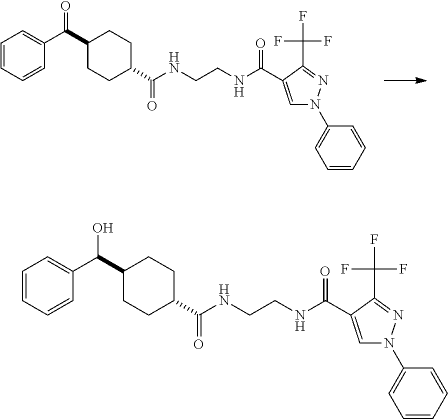

To N-(2-(trans-4-benzoylcyclohexanecarboxamido)ethyl)-1-phenyl-3-(trifluoromethyl)-1H-pyrazole-4-carboxamide (400 mg, 0.78 mmol) in MeOH (20 mL) was added sodium borohydride (30 mg, 0.78 mmol). The mixture was stirred at room temperature for 20 hours then water (30 mL) was added, and the mixture was concentrated. The aqueous layer was extracted with DCM (4×50 mL) and the combined organic layers were washed with brine and dried over MgSO₄. Concentration and purification by MPLC (DCM/MeOH 96/4) yielded N-(2-(trans-4-(hydroxy(phenyl)methyl)cyclohexanecarboxamido)ethyl)-1-phenyl-3-(trifluoromethyl)-1H-pyrazole-4-carboxamide as a white solid (289 mg, 72%). Crystallized from AcOEt/hexanes: ¹H NMR (400 MHz, DMSO-d₆) δ 0.91-1.04 (m, 2H), 1.15-1.47 (m, 4H), 1.63-1.77 (m, 2H), 1.83-1.90 (m, 1H), 1.92-2.00 (m, 1H), 3.14-3.29 (m, 4H), 4.23 (t, 1H, J=5.5 Hz), 5.07 (d, 1H, J=4.3 Hz), 7.18-7.32 (m, 5H), 7.45-7.50 (m, 1H), 7.57-7.64 (m, 2H), 7.74-7.83 (m, 3H), 8.34 (t, 1H, J=5.5 Hz), 9.02 (s, 1H); m/z (APCI pos) 497.1 (100%) [M+H−H₂O], 516.0 (20%) [M+H].

### Example E219

### 1-(3,5-Difluorophenyl)-N-(2-(trans-4-(5-isopropyl-1,3,4-oxadiazol-2-yl)cyclohexanecarboxamido)ethyl)-3-(trifluoromethyl)-1H-pyrazole-4-carboxamide

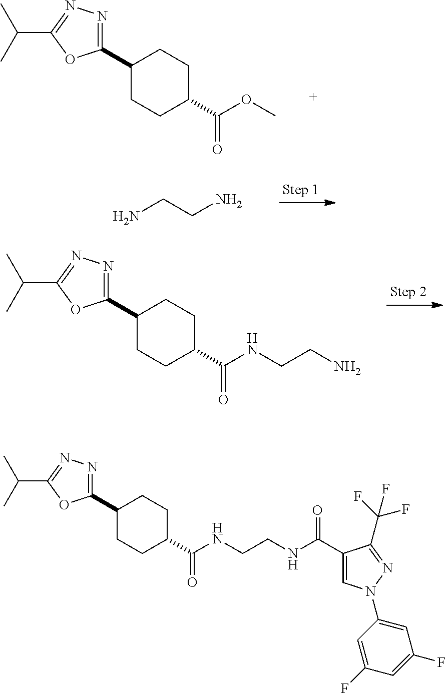

### Step 1

A solution of trans-methyl 4-(5-isopropyl-1,3,4-oxadiazol-2-yl)cyclohexanecarboxylate (0.200 g, 0.793 mmol) in ethylenediamine (2.65 mL, 39.6 mmol) was heated to reflux for 16 h. The ethylenediamine was removed under vacuum to give trans-N-(2-aminoethyl)-4-(5-isopropyl-1,3,4-oxadiazol-2-yl)cyclohexanecarboxamide (0.222 g, quant.) as an oil: ¹H NMR (400 MHz, DMSO-d₆) δ 1.27 (d, 6H, J=7.0 Hz), 1.47 (m, 4H), 1.81 (m, 2H), 2.09 (m, 3H), 2.86 (m, 2H), 3.03 (m, 2H), 3.14 (m, 1H), 7.25 (m, 2H), 7.76 (m, 1H); m/z (APCI pos) 281.1 (100%) [M+H].

### Step 2

To a solution of EDAC.HCl (0.115 g, 0.599 mmol), HOBt.H₂O (0.0918 g, 0.599 mmol), and 1-(3,5-difluorophenyl)-3-(trifluoromethyl)-1H-pyrazole-4-carboxylic acid (0.175 g, 0.599 mmol) in DMF was added trans-N-(2-aminoethyl)-4-(5-isopropyl-1,3,4-oxadiazol-2-yl)cyclohexanecarboxamide (0.140 g, 0.499 mmol). The mixture was stirred for 16 hours at room temperature. The solvent was removed and the residue partitioned between AcOEt and water. The organic layer was dried (sodium sulfate), filtered, and concentrated in vacuo. The residue was triturated with ether/methanol, then purified by chromatography on silica gel (5% MeOH/DCM) to give 1-(3,5-difluorophenyl)-N-(2-(trans-4-(5-isopropyl-1,3,4-oxadiazol-2-yl)cyclohexanecarboxamido) ethyl)-3-(trifluoromethyl)-1H-pyrazole-4-carboxamide (0.068 g, 25% yield) as a solid: ¹H NMR (400 MHz, DMSO-d₆) δ 1.27 (d, 6H, J=7.0 Hz), 1.47 (m, 4H), 1.83 (m, 2H), 2.09 (m, 3H), 2.86 (m, 1H), 3.14 (m, 1H), 3.21 (m, 2H), 3.28 (m, 2H), 7.43 (m, 1H), 7.62 (m, 2H), 7.88 (m, 1H), 8.37 (m, 1H), 9.13 (m, 1H); m/z (APCI pos) 555.1 (100%) [M+H].

Compounds of the following structures were prepared from the corresponding acids and trans-N-(2-aminoethyl)-4-(5-isopropyl-1,3,4-oxadiazol-2-yl)cyclohexanecarboxamide, using a similar method to that described above.

| Ex # | Structure | Name | Physical Data |
|---|---|---|---|
| E220 | 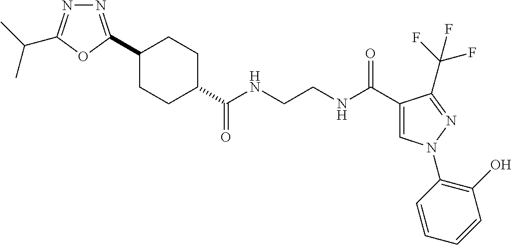 | 1-(2- hydroxyphenyl)- N-(2- (trans-4-(5- isopropyl- 1,3,4- oxadiazol-2- yl)cyclohexane- carboxamido) ethyl)-3- (trifluoro- methyl)-1H- pyrazole-4- carboxamide | ¹H NMR (400 MHz, CD₃OD) δ 1.35 (d, 6H, J = 6.6 Hz), 1.59 (m, 4H), 1.96 (m, 2H), 2.22 (m, 3H), 2.90 (m, 1H), 3.17 (m, 1H), 3.39 (m, 2H), 3.46 (m, 2H), 6.98 (m, 1H), 7.05 (m, 1H), 7.28 (m, 1H), 7.64 (m, 1H), 8.72 (s, 1H); m/z )APCI pos) 535.2 (100%) [M + 1]. |
| E234 | 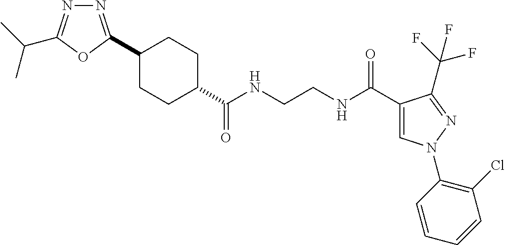 | 1-(2- chlorophenyl)- N-(2- (trans-4-(5- isopropyl- 1,3,4- oxadiazol-2- yl)cyclohexane- carboxamido) ethyl)-3- (trifluoro- methyl)-1H- pyrazole-4- carboxamide | ¹H NMR (400 MHz, CDCl₃) δ 1.37 (d, 6H, J = 7.03 Hz), 1.59 (m, 4H), 2.02 (m, 2H), 2.20 (m, 3H), 2.84 (m, 1H), 3.15 (m, 1H), 3.52 (m, 2H), 3.61 (m, 2H), 6.24 (m, 1H), 6.79 (m, 1H), 7.45 (m, 2H), 7.58 (m, 2H), 8.31 (m, 1H); m/z (APCI pos) 553.1 (100%) (M + H). |

### Example E228

### N-(2-(trans-4-(5-Isopropyl-1,2,4-oxadiazol-3-yl)cyclohexanecarboxamido)ethyl)-1-phenyl-3-(trifluoromethyl)-1H-pyrazole-4-carboxamide

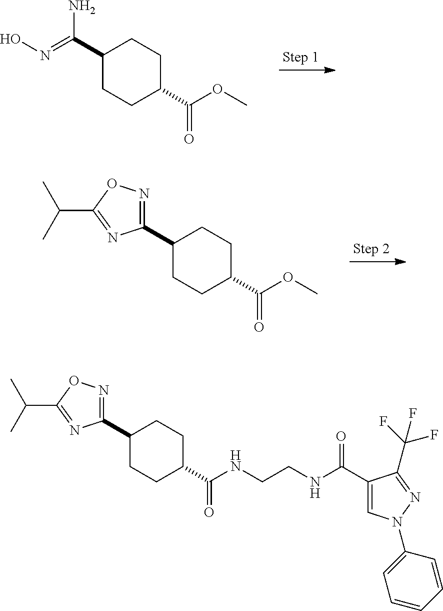

### Step 1

To isobutyric acid (132 mg, 1.50 mmol) in DMF (5 mL) were added diisopropylethylamine (173 mg, 1.50 mmol) and tetramethylfluoroformamidinium hexafluorophosphate (396 mg, 1.50 mmol) at room temperature. The mixture was stirred at room temperature for 30 minutes then trans-methyl 4-(N′-hydroxycarbamimidoyl)cyclohexanecarboxylate (300 mg, 1.50 mmol) was added at once. The mixture was stirred at 110° C. for 3 hours then concentrated to dryness. The residue was dissolved in AcOEt (50 mL) and washed with 1N HCl (2×50 mL) and brine. The organic layer was dried over MgSO₄ then concentrated to yield trans-methyl 4-(5-isopropyl-1,2,4-oxadiazol-3-yl)cyclohexanecarboxylate as a clear oil (397 mg, quant.): ¹H NMR (400 MHz, CDCl₃) δ 1.40 (d, 6H, J=7.0 Hz), 1.50-1.66 (m, 4H), 2.05-2.20 (m, 4H), 2.34-2.41 (m, 1H), 2.72-2.81 (m, 1H), 3.17-3.26 (m, 1H), 3.69 (s, 3H).

### Step 2

trans-Methyl 4-(5-isopropyl-1,2,4-oxadiazol-3-yl)cyclohexanecarboxylate (389 mg, 1.54 mmol) was heated to reflux in ethylenediamine (10 mL) for 18 hours then concentrated to dry ness. Toluene (20 mL) was added and the suspension was concentrated to dryness under high vacuum to give trans-N-(2-aminoethyl)-4-(5-isopropyl-1,2,4-oxadiazol-3-yl)cyclohexanecarboxamide as a beige solid (410 mg, 95%): m/z (APCI pos) (100%) 281.1 [M+H]. To trans-N-(2-aminoethyl)-4-(5-isopropyl-1,2,4-oxadiazol-3-yl)cyclohexanecarboxamide (400 mg, 1.43 mmol) in DCM/DMF (100 mL/10 mL) were successively added 1-phenyl-3-(trifluoromethyl)-1H-pyrazole-4-carboxylic acid (365 mg, 1.43 mmol), EDAC.HCl (356 mg, 1.85 mmol), HOBt.H₂O (270 mg, 2.00 mmol) and triethylamine (289 mg, 2.85 mmol). The mixture was stirred at room temperature for 18 hours then it was concentrated to dryness, and DCM was added (200 mL). The organic layer was successively washed with 2N HCl (100 mL), 10% potassium carbonate aqueous solution (100 mL) and brine (2×200 mL). The organic layer was dried over MgSO₄ then concentrated to yield 546 mg of a white solid which was purified by MPLC: Biotage C18HS 25+M. Method 50-80% linear, 12 mL fractions over 900 ml, flow ˜38 mL/min. Water+1% iPrOH+10 mM NH₄OAc; CH₃CN+1% water+1% iPrOH+10 mM NH₄OAc. 546 mg dissolved in 3 mL hot iPrOH and added on a Samplet™ (precolumn, Biotage, KP-C18-HS). Column pre-equilibrated with 264 mL of 5/95. 142 mg of a white solid were isolated. Crystallized from AcOEt: ¹H NMR (400 MHz, DMSO-d₆) δ 1.29 (d, 3H, J=7.0 Hz), 1.30 (d, 3H, J=6.7 Hz), 1.39-1.55 (m, 4H), 1.80-1.87 (m, 2H), 1.98-2.04 (m, 2H), 2.10-2.17 (m, 1H), 2.65-2.75 (m, 2H), 3.17-3.32 (m, 4H), 7.45-7.50 (m, 1H), 7.59-7.64 (m, 2H), 7.79-7.83 (m, 2H), 7.87 (t, 1H, J=5.5 Hz), 8.37 (t, 1H, J=5.5 Hz), 9.04 (s, 1H); m/z (APCI pos) (100%) 519.1 [M+H].

### Example E229

### 1-Phenyl-N-(2-(trans-4-(5-phenyl-4H-1,2,4-triazol-3-yl)cyclohexanecarboxamido)ethyl)-3-(trifluoromethyl)-1H-pyrazole-4-carboxamide

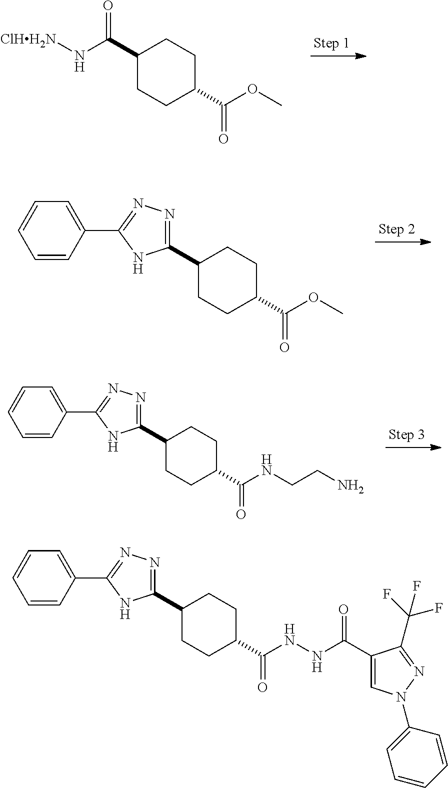

### Step 1

To trans-methyl 4-(hydrazinocarbonyl)cyclohexanecarboxylate hydrochloride (500 mg, 2.11 mmol) in DMF (3 mL) was added benzamidine (305 mg, 2.53 mmol) and the mixture was stirred at 130° C. in a sealed tube for 2 hours. The solvent was evaporated and the residue was purified by MPLC (Biotage Horizon, column C18 25+, 20%->60% A. A: ACN+1% H₂O+1% iPrOH+10 mM NH₄OAc B: H₂O+1% iPrOH+10 mM NH₄OAc) to yield trans-methyl 4-(5-phenyl-4H-1,2,4-triazol-3-yl)cyclohexanecarboxylate as a white solid (200 mg, 26%): ¹H NMR (400 MHz, DMSO-d₆) δ 1.42-1.62 (m, 4H), 1.98-2.12 (m, 4H), 2.34-2.43 (m, 1H), 2.72-2.82 (m, 1H), 3.62 (s, 3H), 7.38-7.48 (m, 3H), 7.96-7.99 (m, 2H); m/z (APCI pos) 286.3 (100%) (M+H).

### Step 2

trans-methyl 4-(5-phenyl-4H-1,2,4-triazol-3-yl)cyclohexanecarboxylate (200 mg, 0.701 mmol) in ethylenediamine (4 mL) was heated to reflux for 18 hours then concentrated to yield trans-N-(2-aminoethyl)-4-(5-phenyl-4H-1,2,4-triazol-3-yl)cyclohexanecarboxamide as a brown solid (244 mg, 89%): m/z (APCI pos) 314.1 (100%) [M+H].

### Step 3

To trans-N-(2-aminoethyl)-4-(5-phenyl-4H-1,2,4-triazol-3-yl)cyclohexanecarboxamide (220 mg, 0.702 mmol) in DCM/DMF (100 mL/10 ml) were successively added 1-phenyl-3-(trifluoromethyl)-1H-pyrazole-4-carboxylic acid (180 mg, 0.702 mmol), EDAC.HCl (175 mg, 0.913 mmol), HOBt.H₂O (133 mg, 0.983 mmol) and triethylamine (142 mg, 1.40 mmol). The mixture was stirred at room temperature for 18 hours then it was concentrated to dryness and purified by MPLC: Biotage C18HS 12+M. Method 50-80% linear, 12 mL fractions over 900 mL, flow ˜38 mL/min. Water+1% iPrOH+10 mM NH₄OAc; CH₃CN+1% water+1% iPrOH+10 mM NH₄OAc. 546 mg dissolved in 3 mL hot iPrOH and added on a Samplet™. Column pre-equilibrated with 264 mL of 5/95. 164 mg of a white solid were isolated. Crystallized from AcOEt: ¹H NMR (400 MHz, DMSO-d₆) δ 1.44-1.59 (m, 4H), 1.83-1.89 (m, 2H), 2.04-2.10 (m, 2H), 2.13-2.21 (m, 1H), 2.71-2.79 (m, 1H), 3.19-3.32 (m, 4H), 7.36-7.51 (m, 4H), 7.59-7.64 (m, 2H), 7.80-7.84 (m, 2H), 7.92 (t, 1H, J=5.5 Hz), 7.95-7.99 (m, 2H), 8.40 (t, 1H, J=5.5 Hz), 9.06 (s, 1H), 13.70 (s, 1H); m/z (APCI pos) (100%) 552.2 [M+H].

### Example E231

### 2-(2-Methyl-1H-imidazol-4-yl)-N-(2-(1-phenyl-3-(trifluoromethyl)-1H-pyrazole-4-carboxamido)ethyl)-4,5,6,7-tetrahydrobenzo[d]thiazole-6-carboxamide

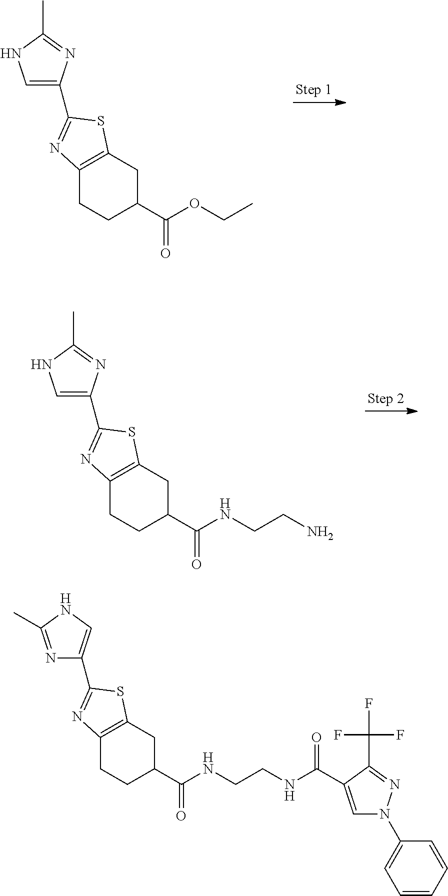

### Step 1

Ethyl 2-(2-methyl-1H-imidazol-4-yl)-4,5,6,7-tetrahydrobenzo[d]thiazole-6-carboxylate (0.200 g, 0.686 mmol) and ethylenediamine (3 mL) were stirred at 75° C. for 4 hours until complete consumption of the starting ester. The solution was concentrated and the residual ethylenediamine was azeotroped off with toluene to afford the crude N-(2-aminoethyl)-2-(2-methyl-1H-imidazol-4-yl)-4,5,6,7-tetrahydrobenzo[d]thiazole-6-carboxamide (0.312 g): m/z (APCI pos) 306.2 [M+H].

Compounds of the following structures were prepared from the corresponding esters, using a similar method to that to described above.

| Structure | Name | Physical Data |
|---|---|---|
| 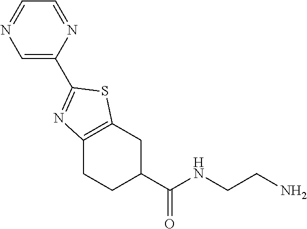 | N-(2- aminoethyl)-2- (pyrazin-2- yl)-4,5,6,7- tetrahydrobenzo [d]thiazole- 6-carboxamide | m/z (APCI pos) 304.1 [M + H] |
| 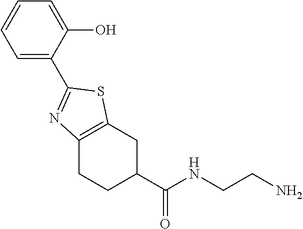 | N-(2- aminoethyl)-2- (2- hydroxyphenyl)- 4,5,6,7- tetrahydrobenzo [d]thiazole- 6-carboxamide | m/z (APCI pos) 318.2 [M + H] |
| 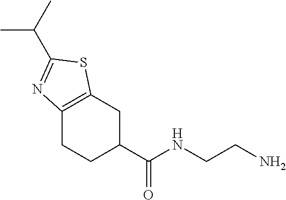 | N-(2- aminoethyl)-2- isopropyl- 4,5,6,7- tetrahydrobenzo [d]thiazole- 6-carboxamide | m/z (APCI pos) 268.1 [M + H] |
| 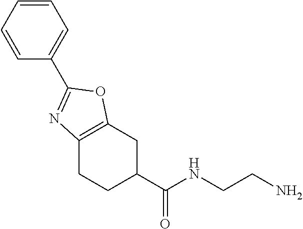 | N-(2- aminoethyl)-2- phenyl- 4,5,6,7- tetrahydrobenzo [d]oxazole-6- carboxamide | m/z (APCI pos) 286.1 [M + H]. |

### Step 2

To N-(2-aminoethyl)-2-(2-methyl-1H-imidazol-4-yl)-4,5,6,7-tetrahydrobenzo[d]thiazole-6-carboxamide (0.312 g, 1 mmol) in DMF (2 mL) were added 1-phenyl-3-(trifluoromethyl)-1H-pyrazole-4-carboxylic acid (0.314 g, 1.23 mmol), HOBt.H₂O (0.188 g, 1.23 mmol) and EDAC.HCl (0.235 g, 1.23 mmol) followed by DIPEA (0.534 mL, 3.06 mmol). The mixture was stirred at room temperature for 18 hours, then quenched with water, and extracted with AcOEt, and the extract was dried and concentrated. Purification by reverse phase HPLC gave 2-(2-methyl-1H-imidazol-4-yl)-N-(2-(1-phenyl-3-(trifluoromethyl)-1H-pyrazole-4-carboxamido)ethyl)-4,5,6,7-tetrahydrobenzo[d]thiazole-6-carboxamide which was crystallized to give a white solid (0.040 g, 7%): ¹H NMR (400 MHz, DMSO-d₆) δ 1.76-1.81 (m, 1H), 2.02-2.08 (m, 1H), 2.28 (s, 3H), 2.56-2.68 (m, 2H), 2.72-2.92 (m, 3H), 3.23-3.35 (m, 4H), 7.46 (d, J=2.4 Hz, 1H), 7.48 (t, J=7.6 Hz, 1H), 7.61 (t, J=8.4 Hz, 2H), 7.81 (d, J=7.2 Hz, 2H), 8.06 (t, 1H), 8.39 (t, 1H), 9.05 (s, 1H), 12.07 (br, 1H); m/z (APCI pos) 544.3 [M+H].

Compounds of the following structures were prepared from the corresponding amines, using a similar method to that described above.

| Ex # | Structure | Name | Physical Data |
|---|---|---|---|
| E221 | 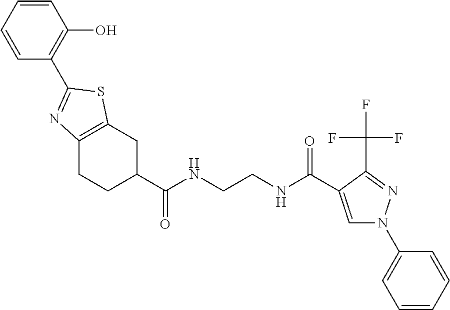 | 2-(2-hydroxyphenyl)-N-(2-(1- phenyl-3-(trifluoromethyl)-1H- pyrazole-4-carboxamido)ethyl)- 4,5,6,7- tetrahydrobenzo[d]thiazole- 6-carboxamide | ¹H NMR (400 MHz, DMSO-d₆) δ 1.80-1.88 (m, 1H), 2.05-2.12 (m, 1H), 2.60-2.69 (m, 2H), 2.83-3.02 (m, 3H), 3.24-3.36 (m, 4H), 6.92 (t, J = 7.6 Hz, 1H), 6.90 (d, J = 8.0 Hz, 1H), 7.26 (t, J = 6.8 Hz, 1H), 7.48 (t, J = 7.6 Hz, 1H), 7.61 (t, J = 8.0 Hz, 2H), 7.81 (d, J = 7.2 Hz, 2H), 7.86 (d, J = 6.8 Hz, 1H), 8.11 (t, J = 5.6 Hz, 1H), 8.40 (t, J = 5.2 H, 1H), 9.05 (s, 1H), 11.45 (s, 1H); m/z (APCI pos) 556.2 [M + H]. |
| E230 | 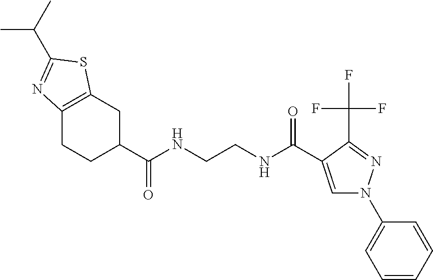 | 2-isopropyl-N-(2-(1-phenyl-3- (trifluoromethyl)-1H-pyrazole- 4-carboxamido)ethyl)-4,5,6,7- tetrahydrobenzo[d]thiazole- 6-carboxamide | ¹H NMR (400 MHz, DMSO-d₆) δ 1.27 (d, J = 6.8 Hz, 6H), 1.73-1.81 (m, 1H), 2.00-2.03 (m, 1H), 2.48-2.68 (m, 2H), 2.71-2.88 (m, 3H), 3.14-3.17 (m, 1H), 3.23-3.38 (m, 4H), 7.47 (t, J = 7.2 Hz, 1H), 7.61 (t, J = 8.0 Hz, 2H), 7.81 (d, J = 7.6 Hz, 2H), 8.05 (t, J = 5.2 Hz, 1H), 8.38 (t, J = 5.6 Hz, 1H), 9.04 (s, 1H); m/z (APCI pos) 506.2 [M + H]. |
| E232 | 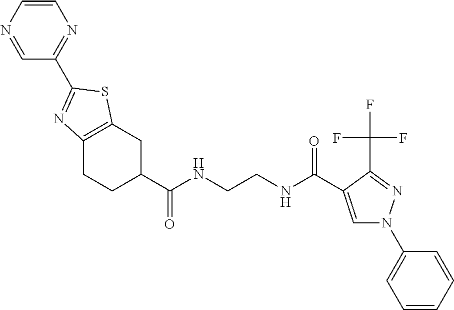 | N-(2-(1-phenyl-3- (trifluoromethyl)-1H-pyrazole- 4-carboxamido)ethyl)-2- (pyrazin-2-yl)-4,5,6,7- tetrahydrobenzo[d]thiazole- 6-carboxamide | ¹H NMR (400 MHz, DMSO-d₆) δ 1.80-1.92 (m, 1H), 2.06-2.14 (m, 1H), 2.62-2.72 (m, 1H), 2.72-2.84 (m, 1H), 2.88-3.08 (m, 3H), 3.24-3.36 (m, 4H), 7.47 (t, J = 7.2 Hz, 1H), 7.60 (t, J = 8.4 Hz, 2H), 7.79 (d, J = 7.6 Hz, 2H), 8.11 (t, J = 5.2 Hz, 1H), 8.38 (t, J = 5.2 Hz, 1H), 8.66-8.69 (m, 2H), 9.04 (s, 1H), 9.22 (d, J = 1.6 Hz, 1H); m/z (APCI pos) 542.2 [M + H]. |
| E233 | 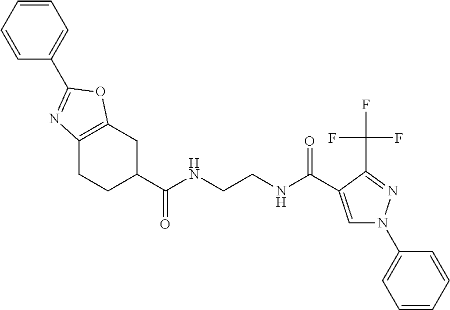 | 2-phenyl-N-(2-(1-phenyl-3- (trifluoromethyl)-1H-pyrazole- 4-carboxamido)ethyl)-4,5,6,7- tetrahydrobenzo[d]oxazole-6- carboxamide | ¹H NMR (400 MHz, DMSO-d₆) δ 1.76-1.83 (m, 1H), 2.00-2.04 (m, 1H), 2.52-2.56 (m, 2H), 2.65-2.75 (m, 1H), 2.82-2.88 (m, 2H), 3.24-3.36 (m, 4H), 7.47-7.53 (m, 4H), 7.61 (t, J = 8.4 Hz, 2H), 7.81 (d, J = 7.6 Hz, 2H), 7.89-7.92 (m, 2H), 8.11 (t, J = 5.2 Hz, 1H), 8.39 (t, J = 5.2 Hz, 1H), 9.05 (s, 1H); m/z (APCI pos) 524.2 [M + H]. |

### Example E242

### 2-Isopropyl-N-(2-(1-phenyl-3-(trifluoromethyl)-1H-pyrazole-4-carboxamido)ethyl)-4,5,6,7-tetrahydrobenzo[d]thiazole-6-carboxamide

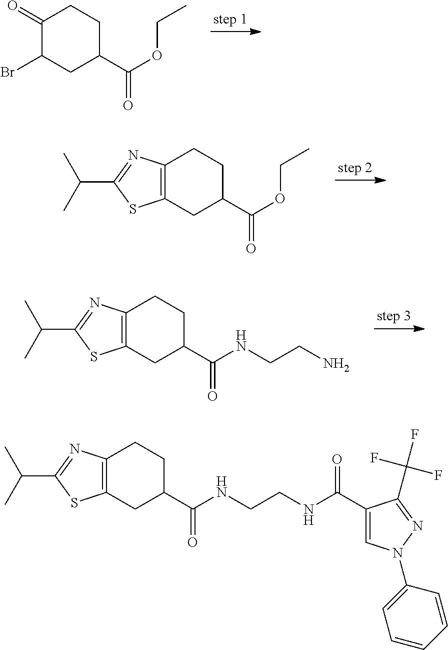

### Step 1

Ethyl 3-bromo-4-oxocyclohexanecarboxylate (1.50 g, 6.02 mmol) in EtOH (7 mL) was charged with 2-methylpropanethioamide (0.62 g, 6.02 mmol, prepared according to Eur. J. Med. Chem. 2004, 39, 867-872.) and the mixture was heated at 120° C. for 15 minutes in open air during which time the solvent evaporated. The solution was cooled and concentrated. The residue was purified by silica gel chromatography to give ethyl 2-isopropyl-4,5,6,7-tetrahydrobenzo[d]thiazole-6-carboxylate (1.04 g, 68%): m/z (APCI pos) 254.2 (100%) (M+H).

The following compound was prepared using the same procedure as described above with isobutyramide as the coupling partner.

| Structure | Name | Physical Data |
|---|---|---|
| 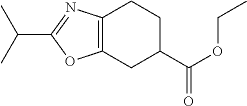 | ethyl 2- isopropyl- 4,5,6,7- tetrahydrobenzo [d]oxazole-6- carboxylate | m/z (APCI pos) 238.2 (100%) (M + H) |

### Step 2

Ethyl 2-isopropyl-4,5,6,7-tetrahydrobenzo[d]thiazole-6-carboxylate (0.30 g, 1.18 mmol) in ethylenediamine (3 mL) was heated at 80° C. for 4 hours. The solution was cooled and azeotroped with toluene to provide the crude N-(2-aminoethyl)-2-isopropyl-4,5,6,7-tetrahydrobenzo[d]thiazole-6-carboxamide (0.32 g, 100%): m/z (APCI pos) 268.1 (100%) (M+H).

The following compound was prepared using the same procedure as described above from ethyl 2-isopropyl-4,5,6,7-tetrahydrobenzo[d]oxazole-6-carboxylate.

| Structure | Name | Physical Data |
|---|---|---|
| 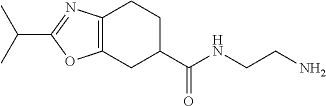 | N-(2-aminoethyl)-2-isopropyl- 4,5,6,7-tetrahydrobenzo[d]oxazole- 6-carboxamide | m/z (APCI pos) 252.1 (100%) (M + H) |

### Step 3

### N-(2-Aminoethyl)-2-isopropyl-4,5,6,7-tetrahydrobenzo[d]thiazole-6-carboxamide (0.19 g, 0.72 mmol), 1-phenyl-3-(trifluoromethyl)-1H-pyrazole-4-carboxylic acid (0.22 g, 0.86 mmol), HOBt.H₂O (0.13 g, 0.86 mmol) and EDAC.HCl (0.16 g, 0.86 mmol) were dissolved in DMF (2 mL). Diisopropylethylamine (0.37 ml, 2.15 mmol) was added last. The mixture was stirred at room temperature overnight. The solution was quenched with water, and extracted with AcOEt, and the extract was dried, and concentrated. Purification by reverse phase HPLC gave 2-isopropyl-N-(2-(1-phenyl-3-(trifluoromethyl)-1H-pyrazole-4-carboxamido)ethyl)-4,5,6,7-tetrahydrobenzo[d]thiazole-6-carboxamide (0.007 g, 1.9%): m/z (APCI pos) 506.2 (M+H); ¹H NMR (400 MHz, DMSO-d₆) δ 1.27 (d, J=6.8 Hz, 6H), 1.73-1.81 (m, 1H), 2.00-2.03 (m, 1H), 2.48-2.68 (m, 2H), 2.71-2.88 (m, 3H), 3.14-3.17 (m, 1H), 3.23-3.38 (m, 4H), 7.47 (t, J=7.2 Hz, 1H), 7.61 (t, J=8.0 Hz, 2H), 7.81 (d, J=7.6 Hz, 2H), 8.05 (t, J=5.2 Hz, 1H), 8.38 (t, J=5.6 Hz, 1H), 9.04 (s, 1H).

The following compound was prepared using the same procedure as described above from N-(2-aminoethyl)-2-isopropyl-4,5,6,7-tetrahydrobenzo[d]oxazole-6-carboxamide.

| Ex # | Structure | Name | Physical Data |
|---|---|---|---|
| E243 | 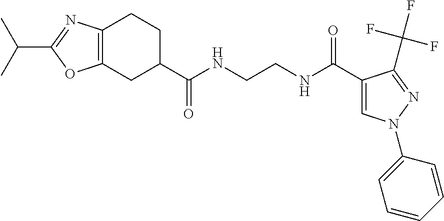 | 2-isopropyl-N-(2-(1-phenyl-3- (trifluoromethyl)-1H-pyrazole- 4-carboxamido)ethyl)-4,5,6,7- tetrahydrobenzo[d]oxazole-6- carboxamide | ¹H NMR (400 MHz, DMSO-d₆) δ 1.22 (d, J = 6.8 Hz, 6H), 1.65-1.76 (m, 1H), 1.94-2.00 (m, 1H), 2.38-2.44 (m, 2H), 2.56-2.64 (m, 1H), 2.67-2.74 (m, 2H), 2.94-3.01 (m, 1H), 3.20-3.36 (m, 4H), 7.48 (t, J = 7.2 Hz, 1H), 7.61 (t, J = 7.6 Hz, 2H), 7.81 (d, J = 7.2 Hz, 2H), 8.05 (t, J = 5.6 Hz, 1H), 8.38 (t, J = 4.8 Hz, 1H), 9.04 (s, 1H); m/z (APCI pos) 490.2 (100%) [M + H] |

### Example E245

### N-(2-(trans-4-(4-Bromo-5-phenyloxazol-2-yl)cyclohexanecarboxamido)ethyl)-1-phenyl-3-(trifluoromethyl)-1H-pyrazole-4-carboxamide

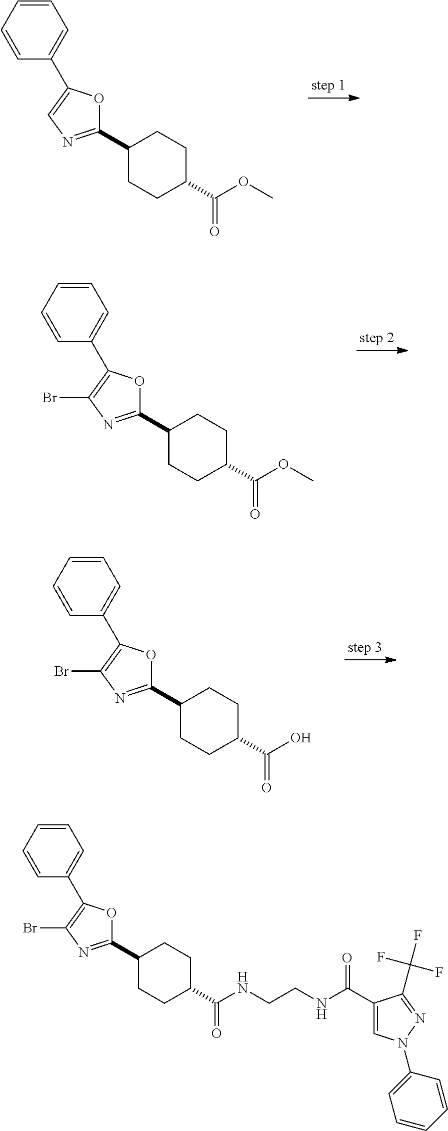

### Step 1

trans-Methyl 4-(5-phenyloxazol-2-yl)cyclohexanecarboxylate (4.23 g, 14.8 mmol) was dissolved in DMF (200 mL). N-Bromosuccinimide (2.77 g, 15.6 mmol) was added and the solution was stirred at 85° C. for 2 hours. The solution was cooled, diluted with water, and extracted with ether, and the extract was dried, and concentrated. The material was recrystallized from AcOEt/hexanes to afford trans-methyl 4-(4-bromo-5-phenyloxazol-2-yl)cyclohexanecarboxylate compound as a pale yellow solid (3.0 g, 55%): m/z (APCI pos) 364.0 (35%) (M+H); ¹H NMR (400 MHz, CDCl₃) δ 1.55-1.67 (m, 4H, 2.14 (d, J=12.0 Hz, 2H), 2.25 (d, J=11.6 Hz, 2H), 2.37 (tt, J=3.2, 11.6 Hz, 1H), 2.82 (tt, J=32, 11.6 Hz, 1H), 3.69 (s, 3H), 7.35 (t, J=7.2 Hz, 1H), 7.44 (t, J=7.6 Hz, 2H), 7.88 (d, J=7.2 Hz, 2H).

### Step 2

NaOH (6.8 ml, 6.8 mmol, 1M) was dissolved in NeOH (2 mL). trans-Methyl 4-(4-bromo-5-phenyloxazol-2-yl)cyclohexanecarboxylate (1.00 g, 2.75 mmol) was added and the mixture was stirred at 70° C. for 90 minutes. The solution was cooled and diluted with saturated aqueous NaHCO₃. The solution was extracted with AcOEt and the aqueous layer was acidified to pH 3. The aqueous solution was then extracted with AcOEt and the organic layer was dried and concentrated to give trans-4-(4-bromo-5-phenyloxazol-2-yl)cyclohexanecarboxylic acid (0.86 g, 89%) as a white solid: m/z (APCI pos) 350.1 (15%) (M+H).

### Step 3

N-(2-(trans-4-(4-Bromo-5-phenyloxazol-2-yl)cyclohexanecarboxamido)ethyl)-1-phenyl-3-(trifluoromethyl)-1H-pyrazole-4-carboxamide was prepared using EDAC.HCl/HOBt.H₂O coupling conditions as described previously from trans-4-(4-bromo-5-phenyloxazol-2-yl)cyclohexanecarboxylic acid and N-(2-aminoethyl)-1-phenyl-3-(trifluoromethyl)-1H-pyrazole-4-carboxamide: m/z (APCI pos) 630.1 (60%) (11+H); ¹H NMR (400 MHz, CDCl₃) δ 1.56-1.72 (m, 4H), 2.00-2.08 (m, 2H), 2.14-2.24 (m, 1H), 2.25-2.32 (m, 2H), 2.78-2.88 (m, 1H), 2.49-3.56 (m, 2H), 3.58-3.64 (m, 2H), 6.72 (br, 1H), 6.79 (br, 1H), 7.34-7.40 (m 1H), 7.44 (t, J=8.0 Hz, 3H), 7.52 (t, J=8.0 Hz, 2H), 7.72 (d, J=9.2 Hz, 2H), 7.79 (d, J=8.4 Hz, 2H), 8.42 (s, 1H).

### Example E246

### 1-Phenyl-N-(2-(trans-4-(2-phenyloxazol-5-yl)cyclohexanecarboxamido)ethyl)-3-(trifluoromethyl)-1H-pyrazole-4-carboxamide

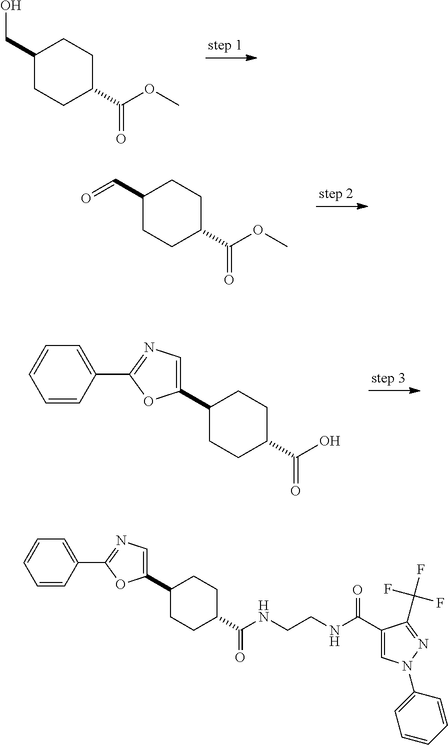

### Step 1

trans-Methyl 4-(hydroxymethyl)cyclohexanecarboxylate (0.750 g, 4.35 mmol) was dissolved in dichloromethane (12 mL). Dess-Martin reagant (1.85 g, 4.35 mmol) was then added. The mixture was stirred for 1 hour at room temperature. The mixture was filtered and the filtrate was diluted with diethyl ether and washed with saturated aqueous NaHCO₃ followed by 10% aqueous sodium thiosulfate. The organic layer was washed with water, brine, dried, and concentrated. The residue was purified by silica gel column chromatography (20% AcOEt/hexanes) to give trans-methyl 4-formylcyclohexanecarboxylate (0.69 g, 93%) as a clear oil. 1H NMR (400 MHz, CDCl₃) δ 1.22-1.38 (m, 2H), 1.43-1.58 (m, 2H), 2.04-2.17 (m, 4H), 2.20-2.35 (m, 2H), 3.68 (s, 3H), 9.63 (s, 1H).

### Step 2

N-((1H-Benzo[d][1,2,3]triazol-1-yl)methyl)benzamide (0.400 g, 1.59 mmol, prepared according to Katritzky, J. Het. Chem. 2002, 39, 759-765.) was dissolved in toluene (5 mL). Phosphorus'pentachloride (0.33 g, 1.6 mmol) was added and the mixture was stirred at 85° C. for 4 hours. The reaction mixture was concentrated in vacuo. The residue was then dissolved in THF (15 mL) and cooled to −40° C. trans-Methyl 4-formylcyclohexanecarboxylate (0.27 g, 1.6 mmol) in THF (3 mL) was added followed by potassium tert-butoxide (0.71 g, 6.3 mmol). The mixture was stirred and allowed to warm up to room temperature. The mixture was then refluxed for an additional 2 hours. The solution was cooled, diluted with water and extracted with AcOEt, and the extract was dried and concentrated to give the crude trans-4-(2-phenyloxazol-5-yl)cyclohexanecarboxylic acid (0.62 g) as a brown residue. The material was used as is in the next step without further purification: m/z (APCI pos) 272.2 (100%) (M+H).

### Step 3

1-Phenyl-N-(2-(trans-4-(2-phenyloxazol-5-yl)cyclohexanecarboxamido)ethyl)-3-(trifluoromethyl)-1H-pyrazole-4-carboxamide was prepared from trans-4-(2-phenyloxazol-5-yl)cyclohexanecarboxylic and N-(2-aminoethyl)-1-phenyl-3-(trifluoromethyl)-1H-pyrazole-4-carboxamide using EDAC.HCl/HOBt.H₂O coupling conditions as described previously: m/z (APCI pos) 552.1 (100%) (M+H); ¹H NMR (400 MHz, CDCl₃) δ 1.56-1.74 (m, 4H), 2.00 (t, J=14.4 Hz, 4H), 2.16 (dt, J=3.2, 12.0 Hz, 1H), 3.03 (dt, J=3.2, 12.0 Hz, 1H), 3.49-3.53 (m, 2H), 3.59-3.63 (m, 2H), 6.27 (br, 1H), 6.79 (br, 1H), 7.30-7.34 (m, 1H), 7.40-7.44 (m, 3H), 7.51 (t, J=7.6 Hz, 2H), 7.59 (d, J=7.6 Hz, 2H), 7.71 (d, J=8.0 Hz, 2H), 7.80 (s, 1H), 8.41 (s, 1H).

### Example E250

### Ethyl 4-fluoro-1-((2-(1-phenyl-3-(trifluoromethyl)-1H-pyrazole-4-carboxamido)ethyl)carbamoyl)piperidine-4-carboxylate

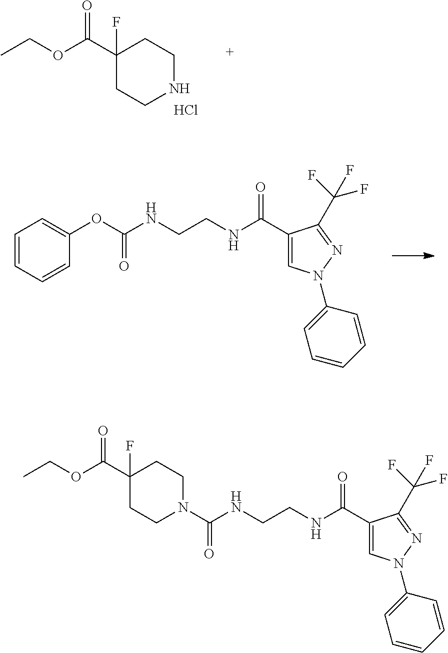

To phenyl 2-(1-phenyl-3-(trifluoromethyl)-1H-pyrazole-4-carboxamido)ethylcarbamate (300 mg, 0.717 mmol) in EtOH (10 mL) were added potassium carbonate (99.1 mg, 0.717 mmol) and ethyl 4-fluoropiperidine-4-carboxylate hydrochloride (304 mg, 1.43 mmol, this compound was obtained from commercially available Boc protected analog and 4N HCl/dioxane) and the mixture was heated to reflux for 24 hours. After cooling down, the mixture was concentrated and the residue was dissolved in DCM and washed successively with 0.1N HCl and saturated aqueous sodium bicarbonate. The organic phase was dried over MgSO₄ and concentrated to give ethyl 4-fluoro-1-((2-(1-phenyl-3-(trifluoromethyl)-1H-pyrazole-4-carboxamido)ethyl)carbamoyl)piperidine-4-carboxylate (287 mg, 80% yield) as a white solid which was crystallized from AcOEt/hexanes: ¹H NMR (400 MHz, DMSO-d₆) δ 1.21 (t, 3H, J=7.0 Hz), 1.78-1.93 (m, 4H), 2.94-3.02 (m, 2H), 3.16-3.23 (m, 2H), 3.25-3.32 (m, 2H), 3.81-3.89 (m, 2H), 4.17 (q, 2H, J=7.0 Hz), 6.77 (t, 1H, J=5.5 Hz), 7.45-7.50 (m, 1H), 7.58-7.64 (m, 2H), 7.79-7.83 (m, 2H), 8.39 (t, 1H, J=5.5 Hz), 9.05 (s, 1H); m/z (APCI pos) 500.1 [M+H].

### Example E251

### 4-Fluoro-1-((2-(1-phenyl-3-(trifluoromethyl)-1H-pyrazole-4-carboxamido)ethyl)carbamoyl)piperidine-4-carboxylic acid

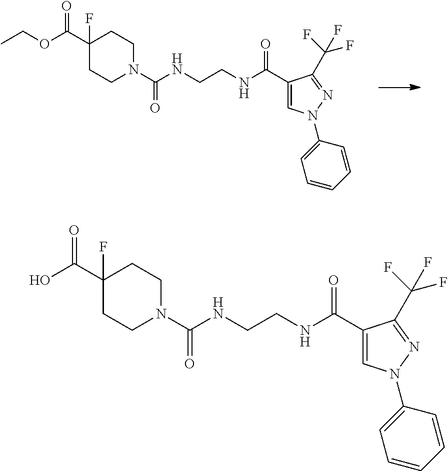

To ethyl 4-fluoro-1-((2-(1-phenyl-3-(trifluoromethyl)-1H-pyrazole-4-carboxamido)ethyl)carbamoyl)piperidine-4-carboxylate (100 mg, 0.200 mmol) in THF/water (1:1 mixture, 10 mL) was added lithium hydroxide hydrate (16.8 mg, 0.400 mmol) and the mixture was stirred at room temperature for 20 hours. The mixture was concentrated to dryness then diluted with water (10 mL) and made acidic with the addition of 2N HCl. The aqueous phase was extracted with AcOEt (4×10 mL) and the combined organic phases were dried over magnesium sulfate, filtered and concentrated to yield 4-fluoro-1-((2-(1-phenyl-3-(trifluoromethyl)-1H-pyrazole-4-carboxamido)ethyl)carbamoyl)piperidine-4-carboxylic acid (93 mg, 98% yield) as a white solid: ¹H NMR (400 MHz, DMSO-d₆) δ 1.76-1.85 (m, 2H), 2.92-3.00 (m, 2H), 3.16-3.22 (m, 2H), 3.26-3.32 (m, 4H), 3.80-3.87 (m, 2H), 6.77 (t, 1H, J=5.5 Hz), 7.45-7.50 (m, 1H), 7.58-7.63 (m, 2H), 7.89-7.83 (m, 2H), 8.41 (t, 1H, J=5.5 Hz), 9.06 (s, 1H); m/z (APCI pos) 472.1 [M+H].

### Example E252

### 1-(2-Fluorophenyl)-N-(2-(trans-4-(5-isopropyl-1,3,4-oxadiazol-2-yl)cyclohexanecarboxamido)ethyl)-3-(trifluoromethyl)-1H-pyrazole-4-carboxamide

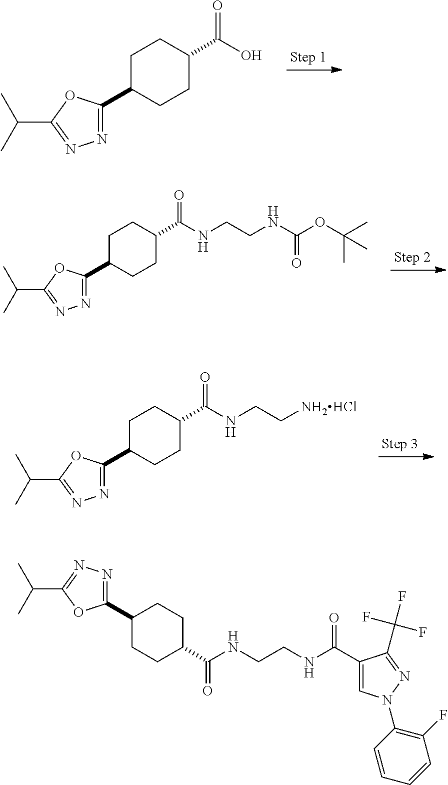

### Step 1

To a solution of trans-4-(5-isopropyl-1,3,4-oxadiazol-2-yl)cyclohexanecarboxylic acid (0.213 g, 0.894 mmol), EDAC.HCl (0.188 g, 0.983 mmol) and HOBt.H₂O (0.151 g, 0.983 mmol) in DMF (1 mL) was added tert-butyl 2-aminoethylcarbamate (0.158 g, 0.983 mmol) in DMF (1 mL). The mixture was stirred for 16 hours at room temperature, diluted with AcOEt, and washed with water, saturated NaHCO₃, and brine. The organic layer was dried (sodium sulfate), filtered, and concentrated in vacuo to give tert-butyl 2-(trans-4-(5-isopropyl-1,3,4-oxadiazol-2-yl)cyclohexanecarboxamido)ethylcarbamate (0.280 g, 82.3% yield) as a solid: ¹H NMR (CDCl₃, 400 MHz) δ 1.37 (d, 6H, J=6.64 Hz), 1.45 (s, 9H), 1.61 (m, 4H), 2.05 (m, 2H), 2.15 (m, 1H), 2.23 (m, 2H), 2.85 (s, 1H), 3.15 (m, 1H), 3.31 (m, 2H), 3.36 (m, 2H), 4.87 (bs, 1H), 6.32 (bs, 1H); m/z (APCI pos) 281.1 [M+H-Boc].

### Step 2

To a solution of tert-butyl 2-(trans-4-(5-isopropyl-1,3,4-oxadiazol-2-yl)cyclohexanecarboxamido)ethylcarbamate (0.280 g, 0.736 mmol) in DCM was added HCl in ether (1M, 1.47 ml, 1.47 mmol). The mixture was stirred for 30 minutes at room temperature, diluted with ether, and filtered. The resulting solid was dried in vacuo to give trans-N-(2-aminoethyl)-4-(5-isipropyl-1,3,4-oxadiazol-2-yl)cyclohexanecarboxamide hydrochloride (0.243 g, quant.) as a solid: ¹H NMR (DMSO-d₆, 400 MHz) δ 1.27 (d, 6H, J=6.64 Hz), 1.48 (m, 4H), 1.81 (m, 4H), 2.84 (m, 2H), 2.95 (m, 1H), 3.04 (m, 1H), 3.14 (m, 1H), 3.26 (m, 2H); m/z (APCI pos) 281.1 [M+H].

### Step 3

To a solution of 1-(2-fluorophenyl)-3-(trifluoromethyl)-1H-pyrazole-4-carboxylic acid (0.078 g, 0.28 mmol) in DMF were added EDAC.HCl (0.055 g, 0.28 mmol) and HOBt.H₂O (0.038 g, 0.28 mmol). Then trans-N-(2-aminoethyl)-4-(5-isopropyl-1,3,4-oxadiazol-2-yl)cyclohexanecarboxamide hydrochloride (0.082 g, 0.26 mmol) and TEA (0.11 ml, 0.78 mmol) were added and the mixture was stirred for 16 h at room temperature. The mixture was diluted with AcOEt and washed with water, saturated sodium bicarbonate, and brine. The organic layers were dried (sodium sulfate), filtered, and concentrated in vacuo. The residue was purified by silica gel column chromatography (3% MeOH/DCM) to give 1-(2-fluorophenyl)-N-(2-(trans-4-(5-isopropyl-1,3,4-oxadiazol-2-yl)cyclohexanecarboxamido)ethyl)-3-(trifluoromethyl)-1H-pyrazole-4-carboxamide (0.008 g, 5.8% yield) as a solid: ¹H NMR (CD₃CN, 400 MHz) δ1.29 (d, 6H, J=7.03 Hz), 1.39 (s, 2H), 1.52 (m, 4H), 1.98 (m, 1H), 2.09 (m, 2H), 3.06-3.18 (m, 2H), 3.33 (m, 2H), 3.39 (m, 2H), 6.60 (m, 1H), 7.09 (m, 1H), 7.21 (m, 1H), 7.57 (m, 3H), 8.53 (s, 1H); m/z (APCI pos) 537.1 [M+H].

### Example E253

### N-(2-(trans-4-(4-Cyano-5-phenyloxazol-2-yl)cyclohexanecarboxamido)ethyl)-1-phenyl-3-(trifluoromethyl)-1H-pyrazole-4-carboxamide

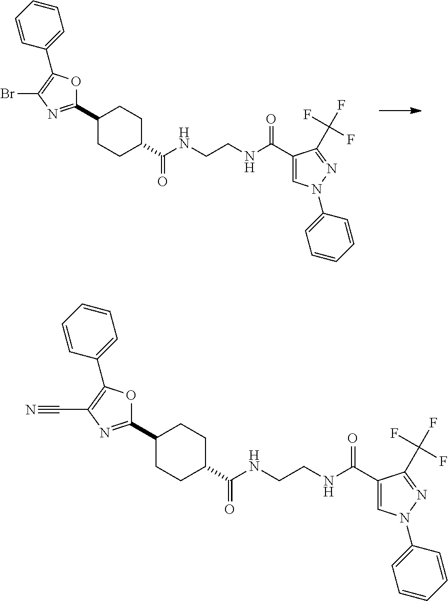

To copper(I) cyanide (0.021 g, 0.24 mmol) in DMF (1 mL) was added N-(2-(trans-4-(4-bromo-5-phenyloxazol-2-yl)cyclohexanecarboxamido)ethyl)-1-phenyl-3-(trifluoromethyl)-1H-pyrazole-4-carboxamide (0.10 g, 0.16 mmol). The reaction was stirred at 150° C. overnight. The solution was cooled, diluted with saturated aqueous NaHCO₃, and extracted with AcOEt, and the extract was dried, and concentrated. Purification by reverse phase HPLC afforded N-(2-(trans-4-(4-cyano-5-phenyloxazol-2-yl)cyclohexanecarboxamido)ethyl)-1-phenyl-3-(trifluoromethyl)-1H-pyrazole-4-carboxamide (0.015 g, 16%) as a white solid: m/z (APCI pos) 577.1 (100%) (M+H); ¹H NMR (400 MHz, CDCl₃) δ 1.60-1.67 (m, 4H), 2.04-2.06 (m, 2H), 2.16-2.19 (m, 1H), 2.25-2.29 (m, 2H), 2.83-2.92 (m, 1H), 3.49-3.56 (m, 2H), 3.58-3.67 (m, 2H), 6.26 (br, 1H), 6.73 (br, 1H), 7.44 (t, J=7.2 Hz, 1H), 7.49-7.54 (m, 5H), 7.71 (d, J=7.6 Hz, 2H), 7.91 (dd, J=1.2, 7.6 Hz, 2H), 8.42 (s, 1H).

### Example E254

### Methyl 5-phenyl-2-(trans-4-(2-(1-phenyl-3-(trifluoromethyl)-1H-pyrazole-4-carboxamido)ethylcarbamoyl)cyclohexyl)oxazole-4-carboxylate

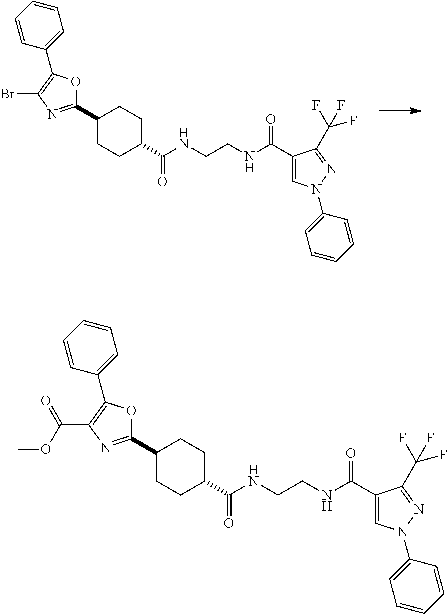

N-(2-(trans-4-(4-Bromo-5-phenyloxazol-2-yl)cyclohexanecarboxamido)ethyl)-1-phenyl-3-(trifluoromethyl)-1H-pyrazole-4-carboxamide (0.10 g, 0.16 mmol), Pd(OAc)₂ (0.005 g, 0.024 mmol), 1,3-bis(diphenylphosphino)propane (dppp) (0.009 g, 0.024 mmol), and K₂CO₃ (0.022 g, 0.16 mmol) were dissolved in MeOH (5 mL). The solution was evacuated and purged with carbon monoxide gas at a balloon pressure. The solution was then heated at 50° C. under the carbon monoxide atmosphere overnight. The solution was cooled, and filtered through celite, and the filtrate was purified by reverse phase HPLC chromatography and the obtained crystals were recrystallized in EtOH/AcOEt to afford methyl 5-phenyl-2-(trans-4-(2-(1-phenyl-3-(trifluoromethyl)-1H-pyrazole-4-carboxamido)ethylcarbamoyl)cyclohexyl)oxazole-4-carboxylate (0.027 g, 27%) as a white solid: m/z (APCI pos) 610.2 (100%) (M+H); ¹H NMR (400 MHz, CDCl₃) δ 1.60-1.76 (m, 4H), 2.03-2.07 (m, 2H), 2.18 (tt, J=3.6, 12.0 Hz, 1H), 2.26-2.29 (m, 2H), 2.89 (tt, J=3.6, 12.0 Hz, 1H), 3.51-3.54 (m, 2H), 3.60-3.63 (m, 2H), 3.92 (s, 3H), 6.23 (br, 1H), 6.76 (br, 1H), 7.42-7.54 (m, 6H), 7.72 (d, J=7.2 Hz, 2H), 8.02-8.05 (m, 2H), 8.41 (s, 1H).

### Example E255

### N-(2-(trans-4-(5-Isopropyl-1,3,4-oxadiazol-2-yl)cyclohexanecarboxamido)ethyl)-1-phenyl-1H-indole-3-carboxamide

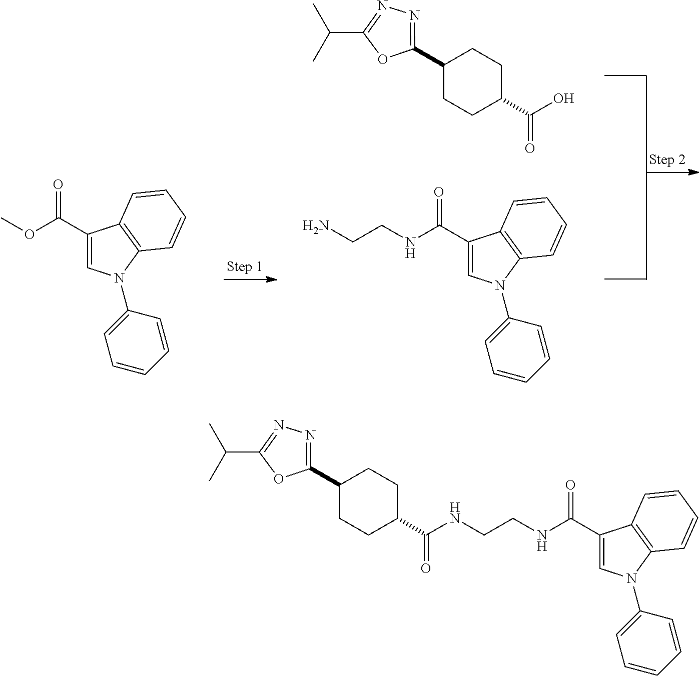

### Step 1

To methyl 1-phenyl-1H-indole-3-carboxylate (500 mg, 1.99 mmol) (prepared according to J. Am. Chem. Soc. 2002, 124(39), 1684) was added ethane-1,2-diamine (120 mg, 1.99 mmol) and the mixture was stirred at 90° C. for 20 hours. The reaction was concentrated to dryness and further dried under high vacuum to give N-(2-aminoethyl)-1-phenyl-1H-indole-3-carboxamide (604 mg, 109% yield) as a tan solid: m/z (APCI pos) 280.0 [M+H].

Compound of the following structure was prepared from methyl 1-(2-fluorophenyl)-1H-indole-3-carboxylate using a similar method to that described above. This compound was treated with 1N HCl/Et₂O and obtained as hydrochloride.

| Structure | Name | Physical Data |
|---|---|---|
| 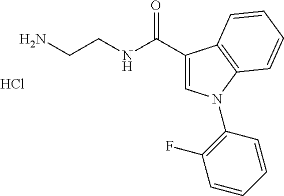 | N-(2-aminoethyl)-1- (2-fluorophenyl)-1H- indole-3-carboxamide hydrochloride | Solid (100%); m/z (APCI pos) 298.0 (100%) [M + H]. |

### Step 2

To trans-4-(5-isopropyl-1,3,4-oxadiazol-2-yl)cyclohexanecarboxylic acid (256 mg, 1.07 mmol) in DCM/DMF (100 mL/10 mL) were successively added N-(2-aminoethyl)-1-phenyl-1H-indole-3-carboxamide (300 mg, 1.07 mmol), EDAC.HCl (206 mg, 1.07 mmol), HOBT.H₂O (145 mg, 1.07 mmol) and triethylamine (109 mg, 1.07 mmol). The mixture was stirred at room temperature for 18 hours then it was concentrated to dryness and the residue was purified by chromatography: Biotage C18HS12+M. Method 20-80% linear, 12 mL fractions over 900 mL, flow ˜38 mL/min. Water+1% IPA+10 mM NH₄OAc; MeCN+1% water+1% IPA+10 mM NH₄OAc. 44 mg of a white solid were isolated. Crystallized from AcOEt/hexanes: ¹H NMR (400 MHz, DMSO-d₆) 1.27 (d, 6H, J=7.0 Hz), 1.40-1.55 (m, 4H), 1.82-1.88 (m, 2H), 2.03-2.10 (m, 2H), 2.11-2.19 (m, 1H), 2.81-2.89 (m, 1H), 3.10-3.18 (rt, 1H), 3.20-3.35 (m, 4H), 7.21-7.29 (m, 2H), 7.46-7.50 (m, 2H), 7.62-7.66 (m, 4H), 7.91 (t, 1H, J=5.5 Hz), 8.10 (t, 1H, J=5.5 Hz), 8.24-8.28 (m, 2H); m/z (APCI pos) 500.2 [M+H].

Compound of the following structure was prepared from N-(2-aminoethyl)-1-(2-fluorophenyl)-1H-indole-3-carboxamide using a similar method to that described above.

| Ex # | Structure | Name | Physical Data |
|---|---|---|---|
| E265 | 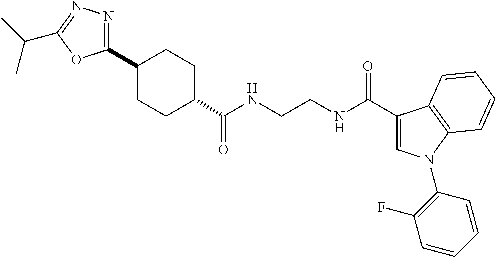 | 1-(2-fluorophenyl)-N-(2- (trans-4-(5-isopropyl- 1,3,4-oxadiazol-2-yl) cyclohexanecarboxamido) ethyl)-1H-indole-3- carboxamide | ¹H NMR (400 MHz, DMSO-d₆) δ 1.27 (d, 6H, J = 6.6 Hz), 1.40-1.53 (m, 4H), 1.81-1.87 (m, 2H), 2.03-2.19 (m, 3H), 2.80-2.90 (m, 1H), 3.10-3.18 (m, 1H), 3.20-3.35 (m, 4H), 7.21-7.27 (m, 3H), 7.44-7.49 (m, 1H), 7.55-7.62 (m, 2H), 7.66-7.72 (m, 1H), 7.91 (t, 1H, J = 5.5 Hz), 8.10 (t, 1H, J = 5.5 Hz), 8.17 (s, 1H), 8.23-8.27 (m, 1H); m/z (APCI pos) 518.2 [M + H]. |

### Example E256

### 1-Phenyl-N-(2-(3-(5-phenyl-1,3,4-oxadiazol-2-yl)cyclopentanecarboxamido)ethyl)-3-(trifluoromethyl)-1H-pyrazole-4-carboxamide

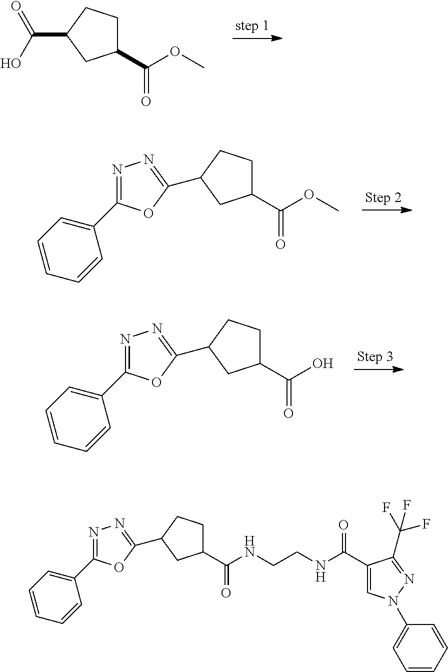

### Step 1

A solution of oxalyl chloride (0.81 g, 6.4 mmol) in DCM (100 ml) was added dropwise to a solution of cis-3-(methoxycarbonyl)cyclopentanecarboxylic acid (1.0 g, 5.8 mmol) and DMF (2 drops) in DCM (50 mL) at room temperature. After stirring for 30 minutes at the same temperature, the solution was concentrated under vacuum to give the acid chloride as an oil. To this oil in DCM (100 mL) were added triethylamine (1.2 g, 12 mmol) and benzohydrazide (0.87 g, 6.4 mmol) and the mixture was stirred at room temperature for 18 hours. The precipitate was collected, and washed with DCM and water. The filtrate was washed with 1N HCl, water and brine then dried over MgSO₄ and concentrated under vacuum to yield cis-methyl 3-(2-benzoylhydrazinocarbonyl)cyclopentanecarboxylate (1.4 g, 83% yield) as a colorless powder. To this powder (1.4 g, 4.8 mmol) in MeCN (20 mL) was added phosphorus oxychloride (0.74 g, 4.8 mmol) and the mixture was stirred at 80° C. for 6 hours. After concentration, the residue was dissolved in AcOEt and the organic phase was washed with saturated aqueous sodium bicarbonate and brine. After drying and concentration, the brown residue was purified by chromatography (DCM/MeOH 98/2) to yield methyl 3-(5-phenyl-1,3,4-oxadiazol-2-yl)cyclopentanecarboxylate as a clear syrup (0.8 g, 61%): ¹H NMR (400 MHz, CDCl₃) δ 1.96-2.58 (m, 6H), 2.95-3.15 (m, 1H), 3.43-3.63 (m, 1H), 3.71 (s, 1.5H), 3.72 (s, 1.5H), 7.47-7.54 (m, 3H), 8.01-8.06 (m, 2H); m/z (APCI pos) 273.1 [M+H].

### Step 2

To methyl 3-(5-phenyl-1,3,4-oxadiazol-2-yl)cyclopentanecarboxylate (0.5 g, 1.84 mmol) in methanol/water (1:1 mixture, 10 mL) was added 2N NaOH (2 mL) and the mixture was stirred at 60° C. for 2 hours. The mixture was concentrated under vacuum, water was added and the pH was brought to 1 by addition of 10% HCl. The aqueous were extracted with AcOEt (3×50 mL) and the combined organic phases were dried over MgSO₄. Filtration and concentration gave 3-(5-phenyl-1,3,4-oxadiazol-2-yl)cyclopentanecarboxylic acid (360 mg, 76% yield) as a clear oil: ¹H NMR (400 MHz, DMSO-d₆) 1.99-2.60 (m, 6H), 2.82-3.19 (m, 1H), 3.43-3.63 (m, 1H), 7.40-7.58 (m, 3H), 8.01-8.06 (m, 2H); m/z (APCI pos) 259.1 [M+H].

### Step 3

To N-(2-aminoethyl)-1-phenyl-3-(trifluoromethyl)-1H-pyrazole-4-carboxamide (346 mg, 1.16 mmol) in DCM/DMF (100 mL/10 mL) were successively added 3-(5-phenyl-1,3,4-oxadiazol-2-yl)cyclopentanecarboxylic acid (300 mg, 1.16 mmol), EDAC.HCl (247 mg, 1.51 mmol) and HOBT.H₂O (220 mg, 1.63 mmol). The mixture was stirred at room temperature for 18 hours then it was concentrated to dryness. DCM (100 mL) was added and the organic phase was successively washed with 2N HCl, 10% aqueous potassium carbonate and brine. After drying and concentration, the residue was purified by chromatography (DCM/MeOH 97/3) to yield 1-phenyl-N-(2-(3-(5-phenyl-1,3,4-oxadiazol-2-yl)cyclopentanecarboxamido)ethyl)-3-(trifluoromethyl)-1H-pyrazole-4-carboxamide (459 mg, 73% yield) as a white solid.

Crystallized from AcOEt/hexanes: ¹H NMR (400 MHz, DMSO-d₆) δ 1.78-2.36 (m, 6H), 2.74-2.90 (m, 1H), 2.30-2.35 (m, 4H), 3.43-3.63 (m, 1H), 7.44-7.50 (m, 1H), 7.56-7.63 (m, 5H), 7.78-7.83 (m, 2H), 7.95-8.03 (m, 3H), 8.38 (t, 1H, J=5.5 Hz), 9.02-9.06 (m, 1H); m/z (APCI pos) 539.2 [M+H].

### Example E257

### 1-(2-Chlorophenyl)-N-(2-(trans-4-(5-isopropyl-1,2,4-oxadiazol-3-yl)cyclohexanecarboxamido)ethyl)-3-(trifluoromethyl)-1H-pyrazole-4-carboxamide

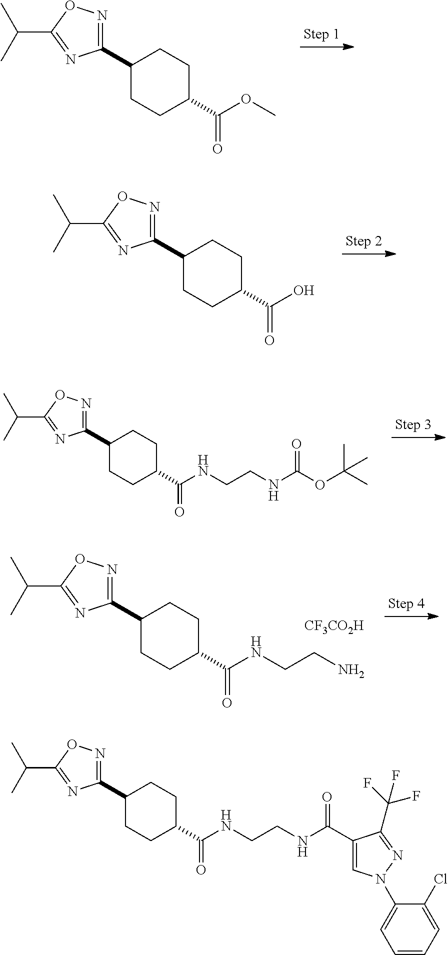

### Step 1

A mixture of trans-methyl 4-(5-isopropyl-1,2,4-oxadiazol-3-yl)cyclohexanecarboxylate (1.0 g, 4.0 mmol), 1N NaOH (7.9 mL, 7.9 mmol) and MeOH (20 mL) was stirred at 50° C. for 16 h. After cooling to room temperature, the mixture was concentrated in vacuo. The residue was diluted with AcOEt, washed successively with 1N HCl and brine, dried and concentrated in vacuo to give trans-4-(5-isopropyl-1,2,4-oxadiazol-3-yl)cyclohexanecarboxylic acid (0.94 g, 100%) as a colorless solid. ¹H NMR (400 MHz, CDCl₃) δ 1.39 (6H, d, J=7.2 Hz), 1.52-1.68 (4H, m), 2.12-2.24 (4H, m), 2.41 (1H, m), 2.77 (1H, m), 3.20 (1H, m); m/z (APCI neg) 237.1 (40%) (M−H).

### Step 2

A mixture of trans-4-(5-isopropyl-1,2,4-oxadiazol-3-yl)cyclohexanecarboxylic acid (0.94 g, 3.95 mmol), tert-butyl 2-aminoethylcarbamate (0.70 g, 4.34 mmol), EDAC.HCl (1.13 g, 5.92 mmol), HOBt.H₂O (0.91 g, 5.92 mmol) and triethylamine (0.68 mL, 4.34 mmol) in CH₃CN (30 mL) was stirred for 16 h at room temperature. The mixture was diluted with AcOEt, washed successively with 1N HCl, saturated aqueous NaHCO₃ and brine, dried and concentrated in vacuo. The residue was purified by silica gel column chromatography (AcOEt) to give tert-butyl 2-(trans-4-(5-isopropyl-1,2,4-oxadiazol-3-yl)cyclohexanecarboxamido)ethylcarbamate (1.35 g, 90%) as colorless crystals. ¹H NMR (400 MHz, CDCl₃) δ 1.39 (6H, d, J=7.2 Hz), 1.44 (9H, s), 1.52-1.67 (4H, m), 1.96-2.08 (2H, m), 2.10-2.24 (3H, m), 2.77 (1H, m), 3.19 (1H, m), 3.24-3.40 (4H, m), 4.88 (1H, br), 6.29 (1H, br); m/z (APCI pos) 281.1 (100%) (M+H-Boc).

### Step 3

To a solution of tert-butyl 2-(trans-4-(5-isopropyl-1,2,4-oxadiazol-3-yl)cyclohexanecarboxamido)ethylcarbamate (1.30 g, 3.42 mmol) in DCM (10 mL) was added TFA (10 mL) and the mixture was stirred for 2 h at room temperature. The mixture was concentrated in vacuo and the excess TFA was removed by azeotropic distillation with toluene to give trans-N-(2-aminoethyl)-4-(5-isopropyl-1,2,4-oxadiazol-3-yl)cyclohexanecarboxamide trifluoroacetate (1.30 g, 97%) as pale yellow crystals. ¹H NMR (400 MHz, DMSO-d₆) δ 1.30 (6H, d, J=7.2 Hz), 1.38-1.56 (4H, m), 1.82-1.94 (2H, m), 1.98-2.10 (2H, m), 2.15 (1H, m), 2.72 (1H, m), 2.80-2.90 (2H, m), 3.24-3.32 (2H, m), 7.82 (3H, br), 7.98 (1H, t, J=5.6 Hz); m/z (APCI pos) 281.1 (100%) (M+H—CF₃CO₂H).

### Step 4

A mixture of trans-N-(2-aminoethyl)-4-(5-isopropyl-1,2,4-oxadiazol-3-yl)cyclohexanecarboxamide trifluoroacetate (0.18 g, 0.44 mmol), 1-(2-chlorophenyl)-3-(trifluoromethyl)-1H-pyrazole-4-carboxylic acid (0.086 g, 0.30 mmol), EDAC.HCl (0.085 g, 0.44 mmol), HOBt.H₂O (0.068 g, 0.44 mmol) and triethylamine (0.12 mL, 0.89 mmol) in CH₃CN (10 mL) was stirred for 16 h at room temperature. The mixture was diluted with AcOEt, washed successively with 1N HCl, saturated aqueous NaHCO₃ and brine, dried and concentrated in vacuo. The residue was purified by silica gel column chromatography (AcOEt—AcOEt/MeOH=10/1) to give 1-(2-chlorophenyl)-N-(2-(trans-4-(5-isopropyl-1,2,4-oxadiazol-3-yl)cyclohexanecarboxamido)ethyl)-3-(trifluoromethyl)-1H-pyrazole-4-carboxamide. Recrystallization from hexanes/AcOEt gave the product (0.051 g, 31%) as colorless crystals. ¹H NMR (400 MHz, CDCl₃) δ 1.39 (6H, d, J=6.8 Hz), 1.50-1.66 (4H, m), 1.94-2.08 (2H, m), 2.10-2.24 (3H, m), 2.75 (1H, m), 3.19 (1H, m), 3.46-3.56 (2H, m), 3.56-3.66 (2H, m), 6.21 (1H, br), 6.88 (1H, br), 7.40-7.49 (2H, m), 7.54-7.62 (2H, m), 8.31 (1H, s); m/z (APCI pos) 553.2 (100%) (M+H).

Compounds of the following structures were prepared from the corresponding acids, using a similar method to that described above.

| Ex # | Structure | Name | Physical Data |
|---|---|---|---|
| E258 | 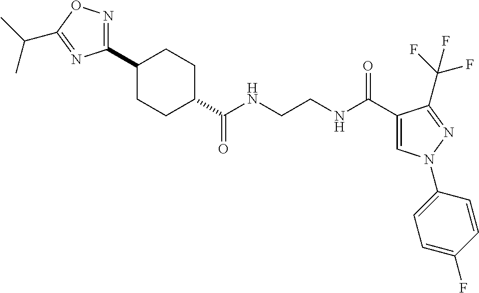 | 1-(4-fluorophenyl)-N-(2- (trans-4-(5-isopropyl-1,2,4- oxadiazol-3-yl) cyclohexanecarboxamido) ethyl)-3-(trifluoromethyl)- 1H-pyrazole-4-carboxamide | ¹H NMR (400MHz, CDCl₃) δ 1.38 (6H, d, J = 6.8 Hz), 1.51- 1.65 (4H, m), 1.92- 2.08 (2H, m), 2.08- 2.26 (3H, m), 2.75 (1H, m), 3.19 (1H, m), 3.44-3.55 (2H, m), 3.55-3.66 (2H, m), 6.29 (1H, t, J = 5.2 Hz), 6.91 (1H, br), 7.21 (2H, t, J = 8.4 Hz), 7.64-7.74 (2H, m), 8.37 (1H, s); m/z (APCI pos) 537.2 (100 %) (M + H). |
| E259 | 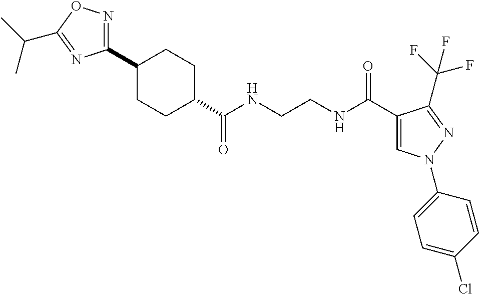 | 1-(4-chlorophenyl)-N-(2- (trans-4-(5-isopropyl-1,2,4- oxadiazol-3-yl) cyclohexanecarboxamido) ethyl)-3-(trifluoromethyl)- 1H-pyrazole-4-carboxamide | ¹H NMR (400MHz, CDCl₃) δ 1.38 (6H, d, J = 6.8 Hz), 1.50-1.66 (4H, m), 1.94-2.08 (2H, m), 2.10-2.24 (3H, m), 2.76 (1H, m), 3.19 (1H, m), 3.46-3.55 (2H, m), 3.55-3.64 (2H, m), 6.21 (1H, t, J = 5.2 Hz), 6.86 (1H, br), 7.49 (2H, d, J = 8.8 Hz), 7.68 (2H, d, J = 8.8 Hz), 8.39 (1H, s); m/z (APCI pos) 553.2 (100%) (M + H). |
| E260 | 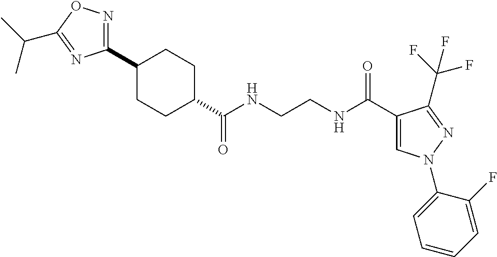 | 1-(2-fluorophenyl)-N-(2- (trans-4-(5-isopropyl-1,2,4- oxadiazol-3-yl) cyclohexanecarboxamido) ethyl)-3-(trifluoromethyl)- 1H-pyrazole-4-carboxamide | ¹H NMR (400MHz, CDCl₃) δ 1.38 (6H, d, J = 6.8 Hz), 1.53-1.66 (4H, m), 1.94-2.06 (2H, m), 2.06-2.26 (3H, m), 2.76 (1H, m), 3.19 (1H, m), 3.46-3.56 (2H, m), 3.56-3.66 (2H, m), 6.26 (1H, t, J = 5.6 Hz), 6.92 (1H, br), 7.28-7.36 (2H, m), 7.41 (1H, m), 7.88 (1H, m), 8.44 (1H, d, J = 1.2 Hz); m/z (APCI pos) 537.2 (100%) (M + H). |

### Example E261

### N-(2-(trans-4-(5-((Cyclopropylmethoxy)methyl)-1,3,4-oxadiazol-2-yl)cyclohexanecarboxamido)ethyl)-1-phenyl-3-(trifluoromethyl)-1H-pyrazole-4-carboxamide

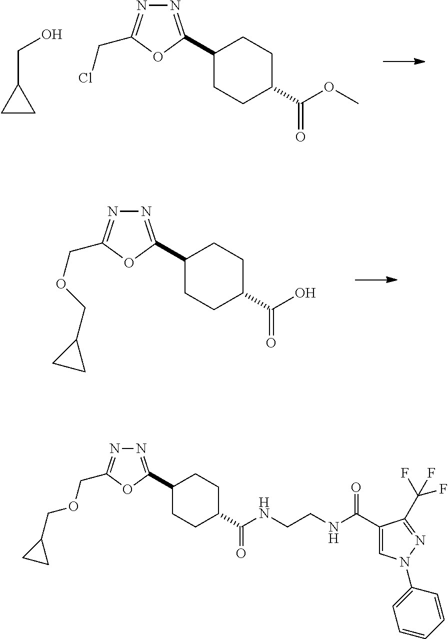

### Step 1

To a mixture of cyclopropylmethanol (0.50 mL, 0.39 mmol) and NaOH (0.031 g, 0.77 mmol) was added trans-methyl 4-(5-(chloromethyl)-1,3,4-oxadiazol-2-yl)cyclohexanecarboxylate (0.10 g, 0.39 mmol) and the mixture was heated at 50° C. overnight. The mixture was concentrated to a residue and dried under high vacuum overnight. The residue was then diluted with water and washed with DCM. The aqueous phase was made acidic with 1N HCl and extracted with AcOEt. The AcOEt extracts were washed with water, dried and concentrated to give trans-4-(5-((cyclopropylmethoxy)methyl)-1,3,4-oxadiazol-2-yl)cyclohexanecarboxylic acid as a residue that was used directly in the next step.

### Step 2

A mixture of N-(2-aminoethyl)-1-phenyl-3-(trifluoromethyl)-1H-pyrazole-4-carboxamide (0.11 g, 0.36 mmol), EDAC.HCl (0.068 g, 0.36 mmol), HOBt.H₂O (0.05463 g, 0.3567 mmol), trans-4-(5-((cyclopropylmethoxyl)methyl)-1,3,4-oxadiazol-2-yl)cyclohexanecarboxylic acid (0.10 g, 0.36 mmol) and DIPEA (0.062 mL, 0.36 mmol) in MeCN (5 mL) was stirred overnight at room temperature. The mixture was concentrated and diluted with DCM, washed with saturated NaHCO₃, dried and concentrated to a residue. The residue was purified by preparative HPLC to give N-(2-(trans-4-(5-((cyclopropylmethoxy)methyl)-1,3,4-oxadiazol-2-yl)cyclohexanecarboxamido)ethyl)-1-phenyl-3-(trifluoromethyl)-1H-pyrazole-4-carboxamide (31.2 mg, 16% (2 steps)) as a light yellow solid: ¹H NMR (400 MHz, DMSO-d₆) δ 0.15-0.19 (m, 2H), 0.45-0.49 (m, 2H), 0.96-1.04 (m, 1H), 1.43-1.56 (m, 4H), 1.81-1.86 (m, 2H), 2.08-2.16 (m, 3H), 2.91-2.96 (m, 1H), 3.22 (t, J=5.9 Hz, 2H), 3.28 (t, J=5.9 Hz, 2H), 3.32-3.38 (m, 2H), 4.66 (s, 2H), 7.48 (t, J=7.3 Hz, 1H), 7.61 (t, J=7.9 Hz, 2H), 7.81 (d, J=7.6 Hz, 2H), 7.90 (t, J=5.6 Hz, 1H), 8.38 (t, J=5.6 Hz, 1H), 9.05 (s, 1H); m/z (APCI pos) 561 (M+H).

Compounds of the following structures were prepared from the corresponding starting materials using a similar method to that described in step 2 of example E261.

| Ex # | Structure | Name | Physical Data |
|---|---|---|---|
| E248 | 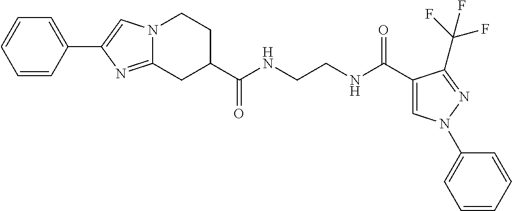 | 2-phenyl-N-(2-(1-phenyl-3- (trifluoromethyl)-1H- pyrazole-4-carboxamido)ethyl)- 5,6,7,8-tetrahydroimidazo[1,2- a]pyridine-7-carboxamide | ¹H NMR (400 MHz, DMSO-d₆) δ 1.90- 2.00 (m, 1H), 2.10-2.16 (m, 1H), 2.76-2.86 (m, 2H), 2.93-2.98 (m, 1H), 3.21-3.37 (m, 4H), 3.86-3.93 (m, 1H), 4.02-4.10 (m, 1H), 7.15 (t, J = 7.4 Hz, 1H), 7.31 (t, J = 7.7 Hz, 2H), 7.40-7.50 (m, 2H), 7.61 (t, J = 7.9 Hz, 2H), 7.69 (d, J = 7.0 Hz, 2H), 7.81 (d, J = 7.6 Hz, 2H), 7.15 (t, J = 5.7 Hz, 1H), 8.41 (t, J = 5.3 Hz, 1H), 9.05 (s, 1H); m/z (APCI pos) 523 (M + H). |
| E247 | 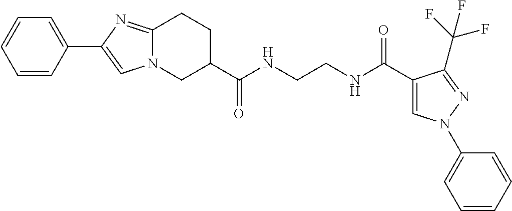 | 2-phenyl-N-(2-(1-phenyl-3- (trifluoromethyl)-1H-pyrazole-4- carboxamido)ethyl)-5,6,7,8- tetrahydroimidazo[1,2-a]pyridine- 6-carboxamide | ¹H NMR (400 MHz, DMSO-d₆) δ 1.88-1.99 (m, 1H), 2.12-2.17 (m, 1H), 2.68-2.76 (m, 1H), 2.82-2.89 (m, 2H), 3.22-3.36 (m, 4H), 3.97-4.06 (m, 1H), 4.11-4.15 (m, 1H), 7.15 (t, J = 7.3 Hz, 1H), 7.31 (t, J = 7.8 Hz, 2H), 7.45-7.50 (m, 2H), 7.61 (t, J = 7.9 Hz, 2H), 7.69 (d, J = 7.0 Hz, 2H), 7.81 (d, J = 7.6 Hz, 2H), 8.23 (t, J = 5.3 Hz, 1H), 8.42 (t, J = 5.4 Hz, 1H), 9.06 (s, 1H); m/z (APCI pos) 523 (M + H). |
| E244 | 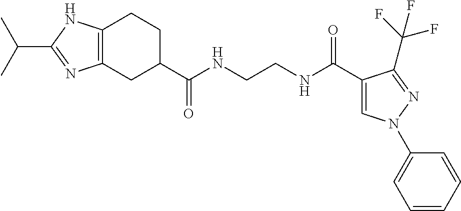 | 2-isopropyl-N-(2-(1-phenyl-3- (trifluoromethyl)-1H-pyrazole-4- carboxamido)ethyl)-4,5,6,7- tetrahydro-1H-benzo[d]imidazole- 5-carboxamide | ¹H NMR (400 MHz, CDCl₃) δ 1.27 (s, 3H), 1.29 (s, 3H), 1.90 (br s, 1H), 2.05-2.08 (m, 1H), 2.55-2.61 (m, 3H), 2.74-2.77 (m, 2H), 2.92-2.99 (m, 1H), 3.49 (d, J = 4.3 Hz, 2H), 3.55 (d, J = 5.1 Hz, 2H), 7.39 (t, J = 7.4 Hz, 1H), 7.49 (t, J = 7.8 Hz, 2H), 7.68 (d, J = 7.8 Hz, 2H), 8.48 (s, 1H); m/z (APCI pos) 489 (M + H). |
| E262 | 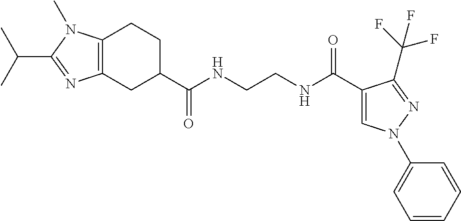 | 2-isopropyl-1-methyl-N-(2-(1- phenyl-3-(trifluoromethyl)-1H- pyrazole-4-carboxamido)ethyl)- 4,5,6,7-tetrahydro-1H- benzo[d]imidazole-5-carboxamide | ¹H NMR (400 MHz, CDCl₃) δ 1.27-1.29 (m, 6H), 1.87-1.97 (m, 1H), 2.10-2.13 (m, 1H), 2.43-2.59 (m, 3H), 2.73-2.83 (m, 2H), 2.93-2.99 (m, 1H), 3.38 (s, 3H), 3.44-3.55 (m, 4H), 6.58 (br s, 1H), 7.20 (br s, 1H), 7.40 (t, J = 7.4 Hz, 1H), 7.49 (t, J = 7.8 Hz, 2H), 7.70 (d, J = 7.8 Hz, 2H), 8.48 (s, 1H) ; m/z (APCI pos) 503 (M + H). |
| E249 | 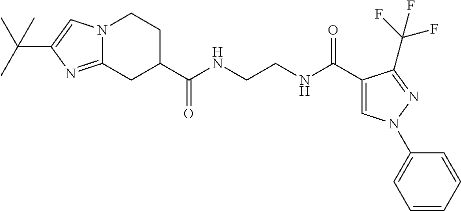 | 2-tert-butyl-N-(2-(1-phenyl-3- (trifluoromethyl)-1H-pyrazole-4- carboxamido)ethyl)-5,6,7,8- tetrahydroimidazo[1,2-a]pyridine- 7-carboxamide | ¹H NMR (400 MHz, DMSO-d₆) δ 1.61 (s, 9H), 1.83-1.99 (m, 1H), 2.06-2.10 (m, 1H), 2.66-2.76 (m, 2H), 2.82-2.88 (m, 1H), 3.20-3.32 (m, 4H), 3.74-3.81 (m, 1H), 3.94-3.98 (m, 1H), 6.63(s, 1H), 7.48 (t, J = 7.4 Hz, 1H), 7.61 (t, J = 7.9 Hz, 2H), 7.82 (d, J = 7.6 Hz, 2H), 8.10 (s, 1H), 8.41 (s, 1H), 9.06 (s, 1H); m/z (APCI pos) 503 (M + H). |

### Example E263

### trans-6-(5-Isopropyl-1,3,4-oxadiazol-2-yl)-1-methyl-N-(2-(1-phenyl-3-(trifluoromethyl)-1H-pyrazole-4-carboxamido)ethyl)piperidine-3-carboxamide

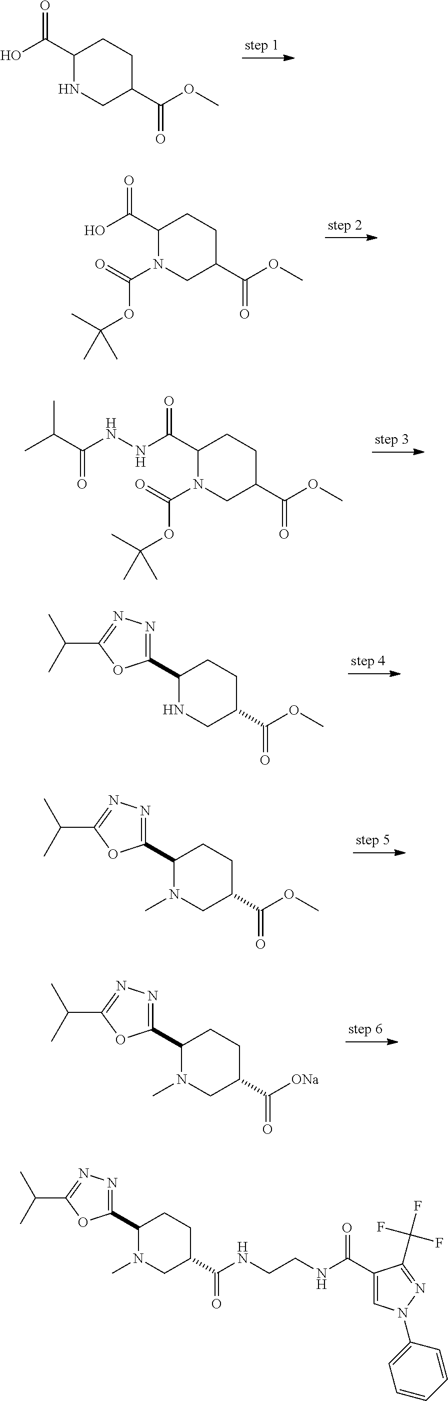

### Step 1

5-(Methoxycarbonyl)piperidine-2-carboxylic acid (6.5 g, 34.7 mmol, prepared as a 3:1 mixture of trans:cis isomers according to Urban, J. Het. Chem. 1995, 32, 857-861) was dissolved in water (100 mL) and the solution was cooled to 0° C. NaOH (36.5 ml, 36.5 mmol, 1M in H₂O) was added followed by di-tert-butyl dicarbonate (11.4 g, 52.1 mmol) in dioxane (125 mL). The reaction was continued to stir at 0° C. for 15 min and then at room temperature overnight. The solution was washed with AcOEt one time. The aqueous layer was acidified to pH=3 using citric acid. The acidified aqueous solution was then extracted with AcOEt, and the extract was dried, and concentrated to provide 1-(tert-butoxycarbonyl)-5-(methoxycarbonyl)piperidine-2-carboxylic acid (10.1 g, 100% yield) as a clear oil: m/z (APCI pos) 188.1 (100%) (M−(Boc)+H).

### Step 2

1-(tert-Butoxycarbonyl)-5-(methoxycarbonyl)piperidine-2-carboxylic acid (10.8 g, 37.8 mmol) was dissolved in acetonitrile (200 mL). Isobutyrohydrazide (4.06 g, 39.7 mmol), EDAC.HCl (7.6 g, 39.7 mmol) and HOBt.H₂O (6.08 g, 39.7 mmol) were added. The mixture was stirred at room temperature overnight. The solution was quenched with 5% citric acid solution, and extracted with AcOEt. The organic layer was washed with saturated aqueous NaHCO₃ and dried and concentrated to afford 1-tert-butyl 3-methyl 6-(2-isobutyrylhydrazinocarbonyl)piperidine-1,3-dicarboxylate as a white solid (10.3 g, 73% yield): m/z (APCI pos) 272.1 (100%) (M-(Boc)+H).

### Step 3

1-tert-Butyl 3-methyl 6-(2-isobutyrylhydrazinocarbonyl)piperidine-1,3-dicarboxylate (4.5 g, 12.1 mmol) was suspended in acetonitrile (120 mL). Phosphorus oxychloride (2.77 ml, 30.3 mmol) was added and the mixture was stirred at 85° C. for 15 hours. The solution was cooled, neutralized with saturated aqueous NaHCO₃, and extracted with chloroform, and the extract was dried, and concentrated. The residue was purified by silica gel column chromatography (5% MeOH/AcOEt) to give trans-methyl 6-(5-isopropyl-1,3,4-oxadiazol-2-yl)piperidine-3-carboxylate as a brown oil (0.50 g, 16% yield): ¹H NMR (400 MHz, CDCl₃) δ 1.38 (d, J=7.2 Hz, 6H), 1.70-1.84 (m, 2H), 2.15-2.28 (m, 2H), 2.61 (tt, J=4.0, 11.0 Hz, 1H-axial CHCO₂Me), 2.91 (t, J=12.4 Hz, 1H), 3.12-3.21 (m, 1H), 3.44-3.50 (m, 1H), 3.70 (s, 3H), 4.01 (dd, J=2.4, 10.4 Hz, 1H-axial CH-oxadiazole); m/z (APCI pos) 253.9 (100%) (M+H).

### Step 4

trans-Methyl 6-(5-isopropyl-1,3,4-oxadiazol-2-yl)piperidine-3-carboxylate (0.18 g, 0.72 mmol) was dissolved in 1,2-dichloroethane and acetonitrile (1:1 mixture, 6 mL). Formaldehyde (0.13 ml, 1.8 mmol, 37% in H₂O) was added followed by acetic acid (0.12 ml, 2.1 mmol). After 15 minutes, sodium triacetoxyborohydride (0.45 g, 2.1 mmol) was added and the mixture was stirred for 15 minutes. The solution was quenched with saturated aqueous NaHCO₃, and extracted with AcOEt, and the extract was dried, and concentrated to afford trans-methyl 6-(5-isopropyl-1,3,4-oxadiazol-2-yl)-1-methylpiperidine-3-carboxylate (0.19 g, 98%) as a brown oil: ¹H NMR (400 MHz, CDCl₃) δ 1.40 (dd, J=3.2, 7.2 Hz, 6H), 1.51-1.62 (m, 1H), 1.82-2.00 (m, 2H), 2.17 (s, 3H), 2.32 (t, J=11.6 Hz, 1H), 2.77 (tt, J=3.2, 11.6 Hz, 1H), 3.15-3.24 (m, 1H), 3.25 (dd, J=2.0, 12.0 Hz, 1H), 3.44 (dd, J=3.2, 10.8 Hz, 1H), 3.71 (s, 3H), 3.64-3.76 (m, 1H); m/z (APCI pos) 268.0 (100%) (M+H).

The following compound was prepared from trans-ethyl 5-(5-isopropyl-1,3,4-oxadiazol-2-yl)piperidine-2-carboxylate using similar conditions as described above.

| Structure | Name | Physical Data |
|---|---|---|
| 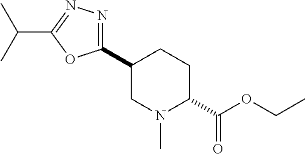 | trans-ethyl 5- (5-isopropyl-1,3,4- oxadiazol-2-yl)-1- methylpiperidine-2- carboxylate | ¹H NMR (400 MHz, CDCl₃) δ 1.31 (t, J = 7.2 Hz, 3H), 1.37 (d, J = 7.2 Hz, 6H), 1.58-1.68 (m, 1H), 1.77-1.86 (m, 1H), 2.03-2.08 (m, 1H) 2.24-2.28 (m, 1H), 2.34 (s, 3H) 2.36-2.40 (m, 1H), 2.76 (d, J = 9.6 Hz, 1H), 3.12-3.19 (m, 1H), 3.20-3.28 (m, 1H), 3.40 (d, J = 11.2 Hz, 1H), 4.25 (q, J = 7.2, 14.4 Hz, 2H); m/z (APCI pos) 282.2 (100%) (M + H) |

### Step 5

trans-Methyl 6-(5-isopropyl-1,3,4-oxadiazol-2-yl)-1-methylpiperidine-3-carboxylate (0.045 g, 0.17 mmol) was dissolved in MeOH. NaOH (0.19 ml, 0.19 mmol, 1M in H₂O) was then added. The mixture was stirred at 65° C. for 3 hours. The solution was cooled, and concentrated, and the residual water was removed by azeotropic distillation with toluene. Sodium trans-6-(5-isopropyl-1,3,4-oxadiazol-2-yl)-1-methylpiperidine-3-carboxylate (0.046 g, 100%) was obtained as a brown solid and used without further purification: m/z (APCI pos) 254.0 (100%) (M-(Na)+H).

The following compound was prepared from the corresponding ester using similar conditions as described above.

| | | |
|---|---|---|
| 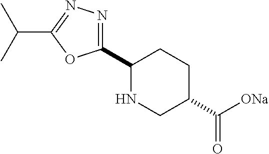 | sodium trans- 6-(5- isopropyl- 1,3,4- yl)piperidine- 3-carboxylate | m/z (APCI pos) 240.0 (100%) (M − (Na) + H) |

### Step 6

trans-6-(5-Isopropyl-1,3,4-oxadiazol-2-yl)-1-methyl-N-(2-(1-phenyl-3-(trifluoromethyl)-1H-pyrazole-4-carboxamido)ethyl)piperidine-3-carboxamide was obtained from N-(2-aminoethyl)-1-phenyl-3-(trifluoromethyl)-1H-pyrazole-4-carboxamide and sodium trans-6-(5-isopropyl-1,3,4-oxadiazol-2-yl)-1-methylpiperidine-3-carboxylate using EDAC.HCl/HOBt.H₂O coupling conditions as described previously: m/z (APCI pos) 534.1 (60%) (M+H); ¹H NMR (400 MHz, CDCl₃) δ 1.39 (dd, J=2.0, 6.8 Hz, 6H), 1.64-1.75 (m, 1H), 1.82-1.92 (m, 1H), 1.97-2.08 (m, 2H), 2.20 (s, 3H), 2.43-2.49 (m, 1H), 2.56-2.64 (m, 1H), 3.12 (dd, J=3.6, 12.0 Hz, 1H), 3.14-3.20 (m, 1H), 3.51-3.54 (m, 2H), 3.61-3.67 (m, 3H), 6.81 (br, 1H), 7.00 (br, 1H), 7.41 (t, J=7.6 Hz, 1H), 7.52 (t, J=7.6 Hz, 2H), 7.71 (d, J=7.6 Hz, 2H), 8.42 (s, 1H).

### Example E264

### N-(2-(3-(5-Isopropyl-1,3,4-oxadiazol-2-yl)pyrrolidine-1-carboxamido)ethyl)-1-phenyl-3-(trifluoromethyl)-1H-pyrazole-4-carboxamide

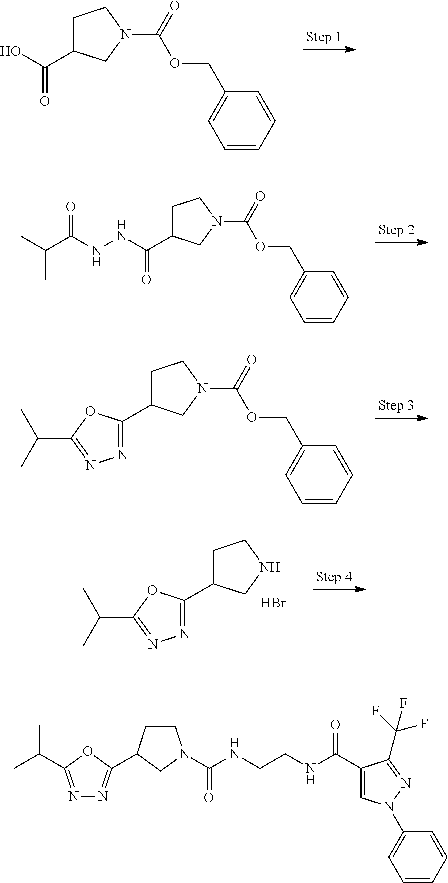

### Step 1

A mixture of isobutyrohydrazide (1.80 g, 17.6 mmol), 1-(benzyloxycarbonyl)pyrrolidine-3-carboxylic acid (4.83 g, 19.4 mmol), EDAC.HCl (4.39 g, 22.9 mmol), HOBt.H₂O (3.51 g, 22.9 mmol) and triethylamine (7.37 mL, 52.9 mmol) in CH₃CN (100 mL) was stirred for 16 h at room temperature. The mixture was diluted with AcOEt, washed successively with 1N HCl, saturated aqueous NaHCO₃ and brine, dried and concentrated in vacuo to give benzyl 3-(2-isobutyrylhydrazinocarbonyl)pyrrolidine-1-carboxylate (4.8 g, 82%) as beige solid. ¹H NMR (400 MHz, CDCl₃) δ 1.18 (6H, d, J=6.8 Hz), 2.06-2.26 (2H, m), 2.49 (1H, m), 3.04 (1H, m), 3.42 (1H, m), 3.52-3.76 (3H, m), 5.13 (2H, d, J=3.2 Hz), 7.27-7.40 (5H, m), 8.62 (1H, br), 9.13 (1H, br); m/z (APCI neg) 332.3 (100%) (M−H).

### Step 2

A mixture of benzyl 3-(2-isobutyrylhydrazinocarbonyl)pyrrolidine-1-carboxylate (2.0 g, 6.00 mmol) and phosphorus oxychloride (0.56 mL, 6.00 mmol) in CH₃CN (40 mL) was heated to reflux for 2 days. After cooling to room temperature, the mixture was diluted with AcOEt, washed successively with saturated aqueous NaHCO₃ and brine, dried and concentrated in vacuo. The residue was purified by silica gel column chromatography (hexanes/AcOEt=1/1-1/2) to give benzyl 3-(5-isopropyl-1,3,4-oxadiazol-2-yl)pyrrolidine-1-carboxylate (0.60 g, 32%) as a colorless oil. ¹H NMR (400 MHz, CDCl₃) δ 1.37 (6H, d, J=6.8 Hz), 2.22-2.42 (2H, m), 3.16 (1H, m), 3.48-3.78 (4H, m), 3.89 (1H, m), 5.15 (2H, d, J=2.0 Hz), 7.28-7.42 (5H, m); m/z (APCI pos) 316.1 (100%) (4+H).

### Step 3

A mixture of benzyl 3-(5-isopropyl-1,3,4-oxadiazol-2-yl)pyrrolidine-1-carboxylate (0.60 g, 1.9 mmol) and hydrogen bromide (33 wt % in AcOH, 10 mL) was stirred for 2 h at room temperature. The mixture was concentrated in vacuo and the excess hydrogen bromide and AcOH were removed by azeotropic distillation with toluene to give 2-isopropyl-5-(pyrrolidin-3-yl)-1,3,4-oxadiazole hydrobromide (0.50 g, 100) as brown amorphous powder. m/z (APCI pos) 182.1 (100%) (M+H—HBr).

### Step 4

A mixture of 2-isopropyl-5-(pyrrolidin-3-yl)-1,3,4-oxadiazole hydrobromide (0.30 g, 1.1 mmol), phenyl 2-(1-phenyl-3-(trifluoromethyl)-1H-pyrazole-4-carboxamido)ethylcarbamate (0.37 g, 0.88 mmol) and cesium carbonate (0.37 g, 1.3 mmol) in EtOH (20 mL) was heated to reflux for 3 h. After cooling to room temperature, the mixture was concentrated in vacuo. The residue was diluted with AcOEt, washed with water and brine, dried and concentrated in vacuo. The residue was purified by silica gel column chromatography (hexanes/AcOEt=2/1-AcOEt-AcOEt/MeOH=10/1) to give N-(2-(3-(5-isopropyl-1,3,4-oxadiazol-2-yl)pyrrolidine-1-carboxamido)ethyl)-1-phenyl-3-(trifluoromethyl)-1H-pyrazole-4-carboxamide. Recrystallization from hexanes/AcOEt gave the product (0.072 g, 16%) as colorless crystals. ¹H NMR (400 MHz, CDCl₃) δ 1.36 (6H, d, J=7.2 Hz), 2.32-2.46 (2H, m), 3.14 (1H, m), 3.42-3.76 (8H, m), 3.82 (1H, m), 5.03 (1H, t, J=5.2 Hz), 7.28 (1H, br), 7.41 (1H, t, J=7.6 Hz), 7.51 (2H, t, J=7.6 Hz), 7.72 (2H, d, J=7.6 Hz), 8.47 (1H, s); m/e (APCI pos) 506.1 (100%) (M+H).

### Example E266

### trans-6-(5-Isopropyl-1,3,4-oxadiazol-2-yl)-N-(2-(1-phenyl-3-(trifluoromethyl)-1H-pyrazole-4-carboxamido)ethyl)piperidine-3-carboxamide

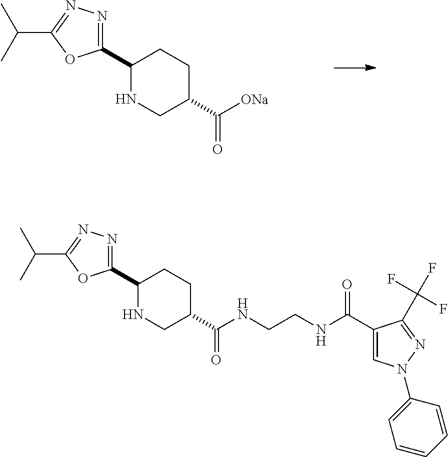

trans-6-(5-Isopropyl-1,3,4-oxadiazol-2-yl)-N-(2-(1-phenyl-3-(trifluoromethyl)-1H-pyrazole-4-carboxamido)ethyl)piperidine-3-carboxamide was prepared from sodium trans-6-(5-isopropyl-1,3,4-oxadiazol-2-yl)piperidine-3-carboxylate and N-(2-aminoethyl)-1-phenyl-3-(trifluoromethyl)-1H-pyrazole-4-carboxamide using similar conditions as described above: m/z (APCI pos) 520.1 (100%) (M+H); ¹H NMR (400 MHz CDCl₃) δ 1.37 (d, J=7.2 Hz, 6H), 1.80-1.85 (M, 3H), 2.00-2.06 (m, 1H), 2.18-2.18 (m, 1H), 2.38-2.45 (m, 1H) 2.95 (dd, J=8.4, 12.0 Hz, 1H), 3.12-3.18 (m, 1H), 3.29 (dd, J=3.6, 12.4 Hz, 1H), 3.50-3.54 (m, 2H), 3.59-3.64 (m, 2H), 4.08 (dd, J=3.6, 8.8 Hz, 1H), 6.84 (br, 1H), 7.07 (br, 1H), 7.42 (t, J=7.2 Hz, 1H), 7.51 (t, J=7.2 Hz, 2H), 7.71 (d, J=8.4 Hz, 2H), 8.44 (s, 1H).

### Example E267

### trans-5-(5-Isopropyl-1,3,4-oxadiazol-2-yl)-1-methyl-N-(2-(1-phenyl-3-(trifluoromethyl)-1H-pyrazole-4-carboxamido)ethyl)piperidine-2-carboxamide

### Example E268

### cis-5-(5-Isopropyl-1,3,4-oxadiazol-2-yl)-1-methyl-N-(2-(1-phenyl-3-(trifluoromethyl)-1H-pyrazole-4-carboxamido)ethyl)piperidine-2-carboxamide

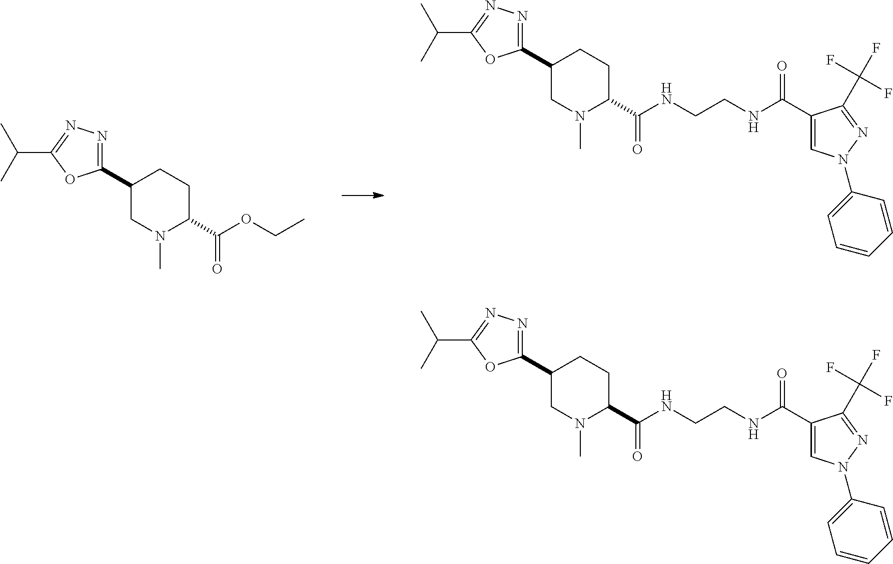

trans-Ethyl 5-(5-isopropyl-1,3,4-oxadiazol-2-yl)-1-methylpiperidine-2-carboxylate (0.10 g, 0.36 mmol) was dissolved in ethanol (2 mL). NaOH (0.36 mL, 0.36 mmol, 1M in H₂O) was added and the mixture was stirred at 65° C. for 24 hours. An additional amount of NaOH (0.090 mL, 0.09 mmol) was then added and the mixture was continued to stir at 65° C. for 2 more hours. The solution was concentrated in vacuo to give the crude sodium 5-(5-isopropyl-1,3,4-oxadiazol-2-yl)-1-methylpiperidine-2-carboxylate salt (0.098 g) as a mixture of cis and trans isomers; m/z (APCI pos) 254.2 (100%) (M-(Na)+H); two peaks. This material was then charged with N-(2-aminoethyl)-1-phenyl-3-(trifluoromethyl)-1H-pyrazole-4-carboxamide (0.14 g, 0.46 mmol), EDAC.HCl (0.89 g, 0.46 mmol) and HOBt.H₂O (0.071 g, 0.46 mmol). The mixture was dissolved in DMF (5 mL) and stirred at room temperature overnight. The solution was quenched with saturated aqueous NaHCO₃, and extracted with AcOEt, and the extract was dried, and concentrated. Purification by reverse phase HPLC gave the trans-5-(5-isopropyl-1,3,4-oxadiazol-2-yl)-1-methyl-N-(2-(1-phenyl-3-(trifluoromethyl)-1H-pyrazole-4-carboxamido)ethyl)piperidine-2-carboxamide isomer (0.025 g, 13%) along with the cis-5-(5-isopropyl-1,3,4-oxadiazol-2-yl)-1-methyl-N-(2-(1-phenyl-3-(trifluoromethyl)-1H-pyrazole-4-carboxamido)ethyl)piperidine-2-carboxamide (0.010 g, 5%). Both isomers were isolated as a white solid: trans isomer: ¹H NMR (400 MHz, CDCl₃) δ 1.37 (d, J=7.2 Hz, 6H), 1.46 (t, J=7.6 Hz, 1H), 1.60 1.72 (m, 1H), 2.10-2.15 (m, 1H), 2.21-2.25 (m, 1H), 2.32 (s, 3H), 2.36-2.41 (m, 2H), 2.66-2.69 (m, 1H), 3.14-3.19 (m, 1H), 3.34 (d, J=12.4 Hz, 1H), 3.56-3.65 (m, 4H), 6.86 (br, 1H), 7.27 (br, 1H), 7.41 (t, J=7.6 Hz, 1H), 7.50 (t, J=7.6 Hz, 2H), 7.70 (d, J=8.4 Hz, 2H), 8.42 (s, 1H); m/z (APCI pos) 534.2 (100%) (M+H); cis isomer: ¹H NMR (400 MHz, CDCl₃) δ 1.35 (d, J=6.8 Hz, 6H), 1.79-2.02 (m, 2H), 2.09-2.14 (m, 1H), 2.34 (s, 3H), 2.64 (d, J=9.2 Hz, 1H), 2.81 (br, 1H), 3.10-3.18 (m, 1H), 3.23-3.28 (m, 2H), 3.48-3.62 (m, 4H), 6.92 (br, 1H), 7.26 (br, 1H), 7.40 (t, J=7.6 Hz, 1H), 7.50 (t, J=7.6 Hz, 2H), 7.76 (d, J=7.6 Hz, 2H), 8.53 (s, 1H); m/z (APCI pos) 534.2 (100%) (M+H).

### Example E269

### 5-Phenyl-2-(trans-4-(2-(1-phenyl-3-(trifluoromethyl)-1H-pyrazole-4-carboxamido)ethylcarbamoyl)cyclohexyl)oxazole-4-carboxylic acid

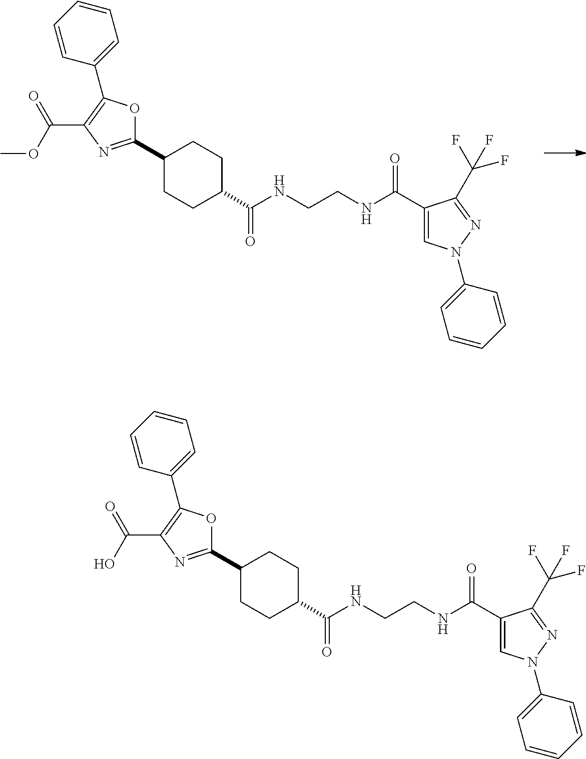

Methyl 5-phenyl-2-(trans-4-(2-(1-phenyl-3-(trifluoromethyl)-1H-pyrazole-4-carboxamido)ethylcarbamoyl)cyclohexyl)oxazole-4-carboxylate (0.20 g, 0.33 mmol) was dissolved in MeOH (5 mL) and K₂CO₃ was added (0.215 g, 1.55 mmol). The solution was stirred at 70° C. overnight. The solution was cooled, diluted with water, and extracted with AcOEt, and the extract was dried, and concentrated. The residue was purified by reverse phase chromatography to afford 5-phenyl-2-(trans-4-(2-(1-phenyl-3-(trifluoromethyl)-1H-pyrazole-4-carboxamido)ethylcarbamoyl)cyclohexyl)oxazole-4-carboxylic acid (0.018 g, 9% yield) as a white solid: m/z (APCI pos) 596.1 (100%) (M+H); ¹H NMR (400 MHz, CD₃OD) δ 1.52-1.72 (m, 4H), 1.87-2.00 (m, 4H), 2.27 (t, J=11.6 Hz, 1H), 2.98 (t, J=12.0 Hz, 1H), 3.39-3.46 (m, 4H), 7.35-7.44 (m, 4H), 7.53 (t, J=8.0 Hz, 2H), 7.81 (d, J=8.8 Hz, 2H), 8.00 (br, 1H), 8.22 (br, 1H), 8.23 (br, 1H), 8.75 (s, 1H).

### 1-(4-Fluorophenyl)-3-(trifluoromethyl)-1H-pyrazole-4-carboxylic acid

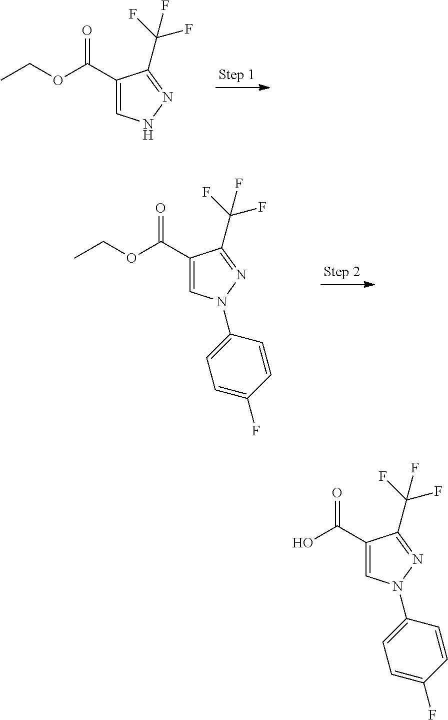

### Step 1

To a solution of ethyl 3-(trifluoromethyl)-1H-pyrazole-4-carboxylate (1.50 g, 7.2 mmol) in DMF were added 4-fluorophenylboronic acid (2.0 g, 14 mmol), copper(II) acetate (0.98 g, 5.4 mmol) and pyridine (1.2 ml, 14 mmol) and the mixture was stirred at room temperature for 2 days. The solvent was removed under vacuum and the residue was partitioned between AcOEt and saturated sodium bicarbonate. The organic layer was washed with brine, dried (sodium sulfate), filtered, and concentrated under vacuum to give ethyl 1-(4-fluorophenyl)-3-(trifluoromethyl)-1H-pyrazole-4-carboxylate (2.3 g, 106% yield) as an oil: ¹H NMR (400 MHz, CDCl₃) δ 1.38 (t, 3H, J=7.0 Hz), 4.37 (q, 2H, J=7.0 Hz), 7.21 (m, 2H), 7.70 (m, 2H), 8.43 (s, 1H).

The following compound was made using the same procedure as described above.

| | | |
|---|---|---|
| 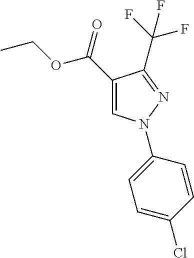 | ethyl 1-(4- chloro- phenyl)-3- (trifluoro- methyl)-1H- pyrazole-4- carboxylate | Solid (87%); ¹H NMR (400 MHz, CDCl₃) δ 1.39 (t, 3H, J = 7.0 Hz), 4.37 (q, 2H, J = 7.0 Hz), 7.49 (m, 2H), 7.68 (m, 2H), 8.47 (m, 1H). |

### Step 2

To a solution of ethyl 1-(4-fluorophenyl)-3-(trifluoromethyl)-1H-pyrazole-4-carboxylate (2.3 g, 7.6 mmol) in EtOH (20 ml) and THF (5 ml) was added 2M sodium hydroxide (11 mL, 23 mmol). The mixture was stirred for 5 minutes at reflux. It was then cooled to room temperature, the solvent was removed under vacuum, and the residue was dissolved in water. This solution was washed with ether, and adjusted to pH 4 with 1N HCl. The resulting solid was filtered to give 1-(4-fluorophenyl)-3-(trifluoromethyl)-1H-pyrazole-4-carboxylic acid (1.62 g, 78% yield) as a powder: ¹H NMR (400 MHz, CDCl₃) δ 7.37 (m, 2H), 7.93 (m, 2H), 9.15 (s, 1H), 13.18 (bs, 1H).

The following compound was made using the same procedure as described above.

| | | |
|---|---|---|
| 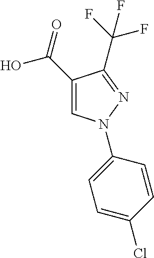 | 1-(4- chlorophenyl)- 3- (trifluorometh- yl)-1H- pyrazole-4- carboxylic acid | Solid (29%); ¹H NMR (400 MHz, CDCl₃) δ 7.64 (m, 2H), 7.98 (m, 2H), 9.26 (s, 1H), 13.27 (bs, 1H). |

### Methyl 2-isopropyl-1-methyl-1H-benzo[d]imidazole-5-carboxylate

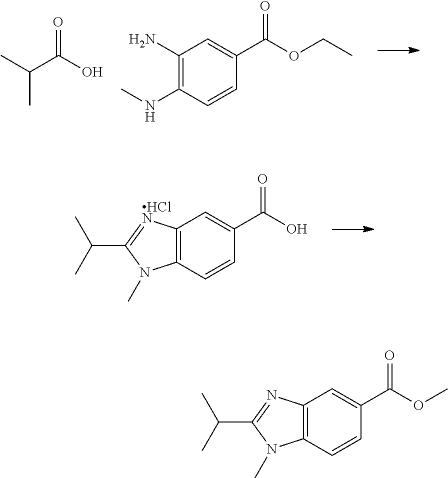

### Step 1

A mixture of ethyl 3-amino-4-(methylamino)benzoate (5.00 g, 25.74 mmol) and isobutyric acid (11.94 mL, 128.7 mmol) in concentrated HCl (50 mL, 25.74 mmol) was heated at 100° C. overnight. The solvent was removed under reduced pressure to give crude 2-isopropyl-1-methyl-1H-benzo[d]imidazole-5-carboxylic acid hydrochloride which was used directly in the next step without further purification.

### Step 2

A mixture of the crude 2-isopropyl-1-methyl-1H-benzo[d]imidazole-5-carboxylic acid hydrochloride in MeOH (100 mL) and concentrated HCl (10.0 mL, 25.74 mmol) was heated at reflux overnight. The solvent was then removed under reduced pressure and water (100 mL) was added to the residue. The pH was adjusted with 1N NaOH to pH 9-10 and the mixture was extracted with AcOEt (3×200 mL). The organic layers were dried over Na₂SO₄, and concentrated to a residue, and the residue was purified by chromatography on silica (50% AcOEt/hexanes containing 1% NH₄OH) to give methyl 2-isopropyl-1-methyl-1H-benzo[d]imidazole-5-carboxylate (2.19 g, 36.6% yield (2 steps)): ¹H NMR (400 MHz, DMSO-d₆) δ 1.34 (d, J=6.8 Hz, 6H), 3.30-3.37 (m, 1H), 3.81 (s, 3H), 3.86 (s, 3H), 7.61 (d, J=8.6 Hz, 1H), 7.85 (d, J=8.4 Hz, 1H), 8.16 (s, 1H); m/z (APCI pos) 233 (M+H).

Compound of the following structure was prepared from the corresponding starting material using a similar method to that described above.

| Structure | Name | Physical Data |
|---|---|---|
| 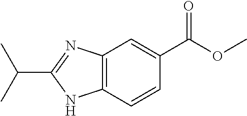 | methyl 2-isopropyl-1H- benzo[d]imidazole-5- carboxylate | ¹H NMR (400 MHz, DMSO-d₆) δ 1.36 (d, J = 7.0 Hz, 6H), 3.16-3.21 (m, 1H), 3.86 (s, 3H), (7.50 (d, J = 8.2 Hz) and 7.61 (d, J = 8.6 Hz), total 1H), 7.75-7.81 (m, 1H), (8.02 (s) and 8.14 (s), total 1H), 12.51 (br s, 1H); m/z (APCI pos) 219 (M + H). |

### Methyl 2-phenylimidazo[1,2-a]pyridine-6-carboxylate

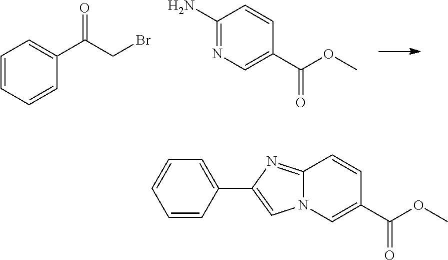

A mixture of methyl 6-aminonicotinate (4.00 g, 26.3 mmol), 2-bromo-1-phenylethanone (5.23 g, 26.3 mmol) and NaHCO₃ (2.65 g, 31.5 mmol) in MeOH (120 mL) was heated at reflux for 18 hours. The mixture was cooled in an ice bath and the resulting solid was filtered to give methyl 2-phenylimidazo[1,2-a]pyridine-6-carboxylate (2.39 g, 36.0% yield) as a tan solid: ¹H NMR (400 MHz, CDCl₃) δ 3.96 (s, 3H), 7.36 (t, J=7.4 Hz, 1H), 7.45 (t, J=7.5 Hz, 2H), 7.62 (d, J=9.4 Hz, 1H), 7.72 (d, J=9.6 Hz, 1H), 7.92 (s, 1H), 7.96 (d, J=7.0 Hz, 2H), 8.90 (s, 1H); m/z (APCI pos) 253 (M+H).

Compounds of the following structures were prepared from ethyl 2-aminoisonicotinate and the corresponding α-bromoketones using a similar method to that described above.

| Structure | Name | Physical Data |
|---|---|---|
| 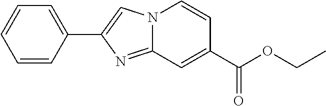 | ethyl 2-phenylimidazo[1,2- a]pyridine-7-carboxylate | ¹H NMR (400 MHz, CDCl₃) δ 1.43 (t, J = 7.1 Hz, 3H), 4.39-4.45 (m, 2H), 7.35-7.41 (m, 2H), 7.46 (t, J = 7.6 Hz, 2H), 7.97 (s, 2H), 7.99 (s, 1H), 8.15 (d, J = 7.0 Hz, 1H), 8.39 (s, 1H); m/z (APCI pos) 267 (M + H). |
| 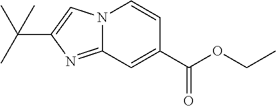 | ethyl 2-tert-butylimidazo[1,2- a]pyridine-7-carboxylate | m/z (APCI pos) 247 (M + H). |

### Methyl 2-phenyl-5,6,7,8-tetrahydroimidazo[1,2-a]pyridine-6-carboxylate

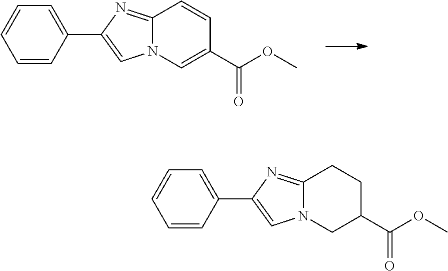

Methyl 2-phenylimidazo[1,2-a]pyridine-6-carboxylate (0.100 g, 0.396 mmol) was dissolved in a mixture of MeOH (20 mL) and concentrated HCl (0.0661 mL, 0.396 mmol), and the reaction pressure vessel was purged with nitrogen. Pd/C (0.169 g, 0.0793 mmol) was added and the vessel was flushed with nitrogen. The vessel was then flushed with H₂ and the mixture was stirred overnight under balloon pressure of H₂. The catalyst was filtered off and the filtrate was concentrated to a residue which was dissolved in DCM, washed with saturated aqueous NaHCO₃, dried over Na₂SO₄, and concentrated to a residue. The obtained residue was purified by chromatography on silica (5% MeOH/DCM) to give methyl 2-phenyl-5,6,7,8-tetrahydroimidazo[1,2-a]pyridine-6-carboxylate (0.104 g, 102% yield) as a white solid: ¹H NMR (400 MHz, DMSO-d₆) δ 1.98-2.07 (m, 1H), 2.18-2.25 (m, 1H), 2.79-2.83 (m, 2H), 3.14-3.21 (m, 1H), 3.68 (s, 3H), 4.08-4.13 (m, 1H), 4.19-4.23 (m, 1H), 7.16 (t, J=7.4 Hz, 1H), 7.32 (t, J=7.7 Hz, 2H), 7.51 (s, 1H), 7.69 (d, J=7.0 Hz, 2H); m/z (APCI pos) 257 (M+H).

Compound of the following structure was prepared from the corresponding starting material using a similar method to that to described above.

| Structure | Name | Physical Data |
|---|---|---|
| 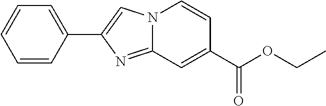 | ethyl 2-phenyl-5,6,7,8- tetrahydroimidazo[1,2- a]pyridine-7-carboxylate | ¹H NMR (400 MHz, CDCl₃) δ 1.29 (t, J = 7.1 Hz, 3H), 2.11-2.21 (m, 1H), 2.35-2.39 (m, 1H), 2.89-2.96 (m, 1H), 3.06-3.13 (m, 1H), 3.26-3.31 (m, 1H), 3.94-4.01 (m, 1H), 4.11-4.17 (m, 1H), 4.18-4.24 (m, 2H), 7.09 (s, 1H), 7.22 (t, J = 7.3 Hz, 1H), 7.35 (t, J = 7.7 Hz, 2H), 7.73 (d, J = 7.2 Hz, 2H); m/z (APCI pos) 271 (M + H). |

### Ethyl 2-tert-butyl-5,6,7,8-tetrahydroimidazo[1,2-a]pyridine-7-carboxylate

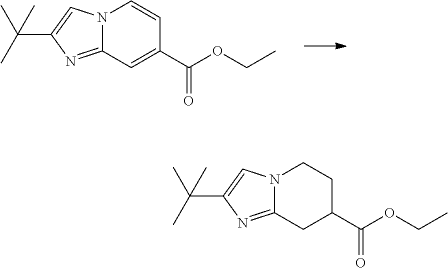

Ethyl 2-tert-butyl imidazo[1,2-a]pyridine-7-carboxylate (0.500 g, 2.03 mmol) was dissolved in a mixture of EtOH (30 mL) and concentrated HCl (0.338 mL, 2.03 mmol), and the reaction pressure vessel was purged with nitrogen. Pd/C (0.864 g, 0.406 mmol) was added to the reaction pressure vessel and the vessel was purged with nitrogen three times. The vessel was then flushed with H₂ three times and pressurized to 250 psi. The mixture was heated in an oil bath at 75° C. for 18 hours then cooled, the catalyst was filtered off and the filtrate was concentrated to a residue. The obtained residue was dissolved in DCM, washed with saturated aqueous NaHCO₃, dried over Na₂SO₄, and concentrated to a residue which was purified by chromatography on silica (75% AcOEt/hexanes) to give ethyl 2-tert-butyl-5,6,7,8-tetrahydroimidazo[1,2-a]pyridine-7-carboxylate (0.325 g, 64.0% yield) as a white solid: ¹H NMR (400 MHz, CDCl₃) δ 1.11-1.34 (m, 12H), 2.03-2.13 (m, 1H), 2.30-2.34% (m, 1H), 2.82-2.89 (m, 1H), 2.96-3.03 (m, 1H), 3.20-3.26 (m, 1H), 3.83-3.90 (m, 1H), 4.01-4.06 (m, 1H), 4.13-4.23 (m, 2H), 6.49 (s, 1H); m/z (APCI pos) 251 (M+H).

Compounds of the following structures were prepared from the corresponding starting materials using a similar method to that described above.

| Structure | Name | Physical Data |
|---|---|---|
| 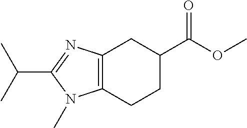 | methyl 2-isopropyl- 1-methyl-4,5,6,7- tetrahydro-1H- benzo[d]imidazole- 5-carboxylate | ¹H NMR (400 MHz, CDCl₃) δ 1.30-1.32 (m, 6H), 1.88-1.98 (m, 1H), 2.19-2.25 (m, 1H), 2.47-2.64 (m, 2H), 2.70-2.84 (m, 2H), 2.87-2.92 (m, 1H), 2.94-3.01 (m, 1H), 3.40 (s, 3H), 3.70 (s, 3H); m/z (APCI pos) 237 (M + H). |
| 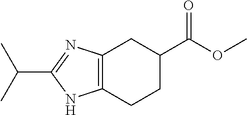 | methyl 2-isopropyl- 4,5,6,7-tetrahydro-1H- benzo[d]imidazole- 5-carboxylate | ¹H NMR (400 MHz, CDCl₃) δ 1.48 (d, J = 7.0 Hz, 6H), 1.92-2.00 (m, 1H), 2.21-2.24 (m, 1H), 2.67-2.96 (m, 4H), 3.00-3.06 (m, 1H), 3.41-3.48 (m, 1H), 3.71 (s, 3H); m/z (APCI pos) 223 (M + H) . |

### 2-Phenyl-5,6,7,8-tetrahydroimidazo[1,2-a]pyridine-6-carboxylic acid hydrochloride

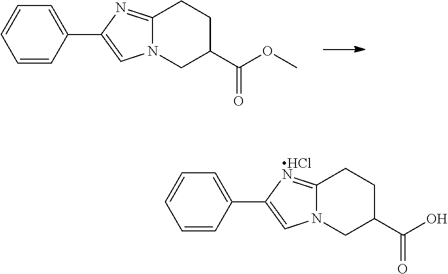

To a mixture of methyl 2-phenyl-5,6,7,8-tetrahydroimidazo[1,2-a]pyridine-6-carboxylate (0.100 g, 0.390 mmol) in water (3 mL) was added concentrated HCl (0.065 mL, 0.390 mmol) and the mixture was heated at reflux for 4 hours. The mixture was concentrated to dryness to give crude 2-phenyl-5,6,7,8-tetrahydroimidazo[1,2-a]pyridine-6-carboxylic acid hydrochloride (0.118 g, 110% yield) that was used directly in the next step without further purification: m/z (APCI neg) 241 (4-H—HCl).

Compounds of the following structures were prepared from the corresponding esters using a similar method to that described above.

| Structure | Name | Physical Data |
|---|---|---|
| 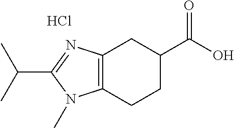 | 2-isopropyl-1-methyl- 4,5,6,7-tetrahydro-1H- benzo[d]imidazole-5- carboxylic acid hydrochloride | m/z (APCI pos) 223 (M + H − HCl). |
| 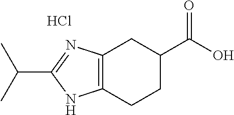 | 2-isopropyl-4,5,6,7- tetrahydro-1H- benzo[d]imidazole-5- carboxylic acid hydrochloride | m/z (APCI pos) 209 (M + H − HCl). |
| 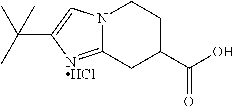 | 2-tert-butyl-5,6,7,8- tetrahydroimidazo[1,2- a]pyridine-7-carboxylic acid hydrochloride | m/z (APCI pos) 223 (M + H − HCl). |
| 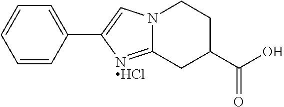 | 2-phenyl-5,6,7,8- tetrahydroimidazo[1,2- a]pyridine-7-carboxylic acid hydrochloride | m/z (APCI pos) 243 (M + H − HCl). |

### trans-Ethyl 5-(5-isopropyl-1,3,4-oxadiazol-2-yl)piperidine-2-carboxylate

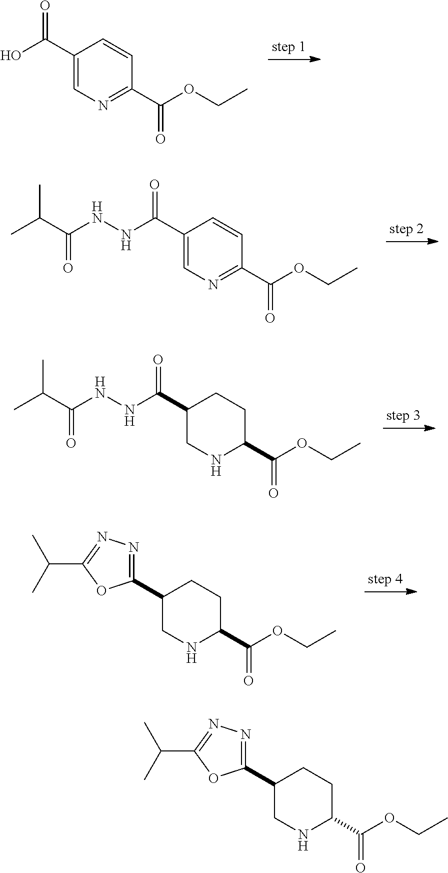

### Step 1

### 6-(Ethoxycarbonyl)nicotinic acid (5.0 g, 25.6 mmol, prepared according to the U.S. Pat. No. 4,273,779), isobutyrohydrazide (2.75 g, 26.9 mmol), EDAC.HCl (5.16 g, 26.9 mmol) and HOBt.H₂O (4.12 g, 26.9 mmol) were dissolved in acetonitrile (50 mL). The solution was stirred at room temperature overnight. The solution was washed with 1N HCl, saturated aqueous NaHCO₃, dried, and concentrated to afford ethyl 5-(2-isobutyrylhydrazinocarbonyl)picolinate as a white solid (3.8 g, 53% yield): m/z (APCI pos) 280.1 (100%) (M+H).

### Step 2

Ethyl 5-(2-isobutyrylhydrazinocarbonyl)picolinate (3.81 g, 13.6 mmol) was suspended in EtOH (200 mL). Platinum(IV) oxide (0.93 g, 4.1 mmol) was added. The solution was evacuated and purged under hydrogen gas and stirred at 50 psi over 2 days at room temperature. The solution was then filtered and the filtrate was concentrated to obtain cis-ethyl 5-(2-isobutyrylhydrazinocarbonyl)piperidine-2-carboxylate (4.6 g) as a clear oil (APCI pos) 286.1 (100%) (M+H).

### Step 3

cis-Ethyl 5-(2-isobutyrylhydrazinocarbonyl)piperidine-2-carboxylate (3.89 g, 13.6 mmol) was dissolved in acetonitrile (130 mL). POCl₃ (5.0 ml, 54.5 mmol) was added and the mixture was stirred at 85° C. for 5 hours. The mixture was cooled, neutralized with saturated aqueous NaHCO₃, and extracted with AcOEt, and the extract was dried, and concentrated. The residue was purified by silica gel column chromatography (5% MeOH/AcOEt) to give cis-ethyl 5-(5-isopropyl-1,3,4-oxadiazol-2-yl)piperidine-2-carboxylate (0.46 g, 13% yield) as a brown oil: ¹H NMR (400 MHz, CDCl₃) δ 1.28 (t, J=7.2 Hz, 3H), 1.37 (d, J=7.2 Hz, 6H), 1.90-1.94 (m, 1H), 1.98 (t, J=6.4 Hz, 1H), 2.02-2.05 (m, 2H), 2.16-2.24 (m, 1H), 3.03-3.08 (m, 1H), 3.14-3.18 (m 2H), 3.33 (dd, J=6.8, 12.4 Hz, 1H), 3.55 (t, J=6.8 Hz, 1H), 4.19 (q, J=6.8, 14.0 Hz, 2H); m/z (APCI pos) 268.1 (100%) (M+H).

### Step 4

cis-Ethyl 5-(5-isopropyl-1,3,4-oxadiazol-2-yl)piperidine-2-carboxylate (0.46 g, 1.72 mmol) was dissolved in acetic acid (5 mL). Salicylaldehyde (0.060 mL) was added. The mixture was stirred at 110° C. for 3 hours to obtain a mixture of the cis and trans isomers (ca 1:1). The solution was cooled and concentrated in vacuo to a thick oil. This oil was neutralized with saturated aqueous NaHCO₃, and extracted with AcOEt, and the extract was dried, and concentrated. The residue was purified by silica gel column chromatography (5% MeOH/AcOEt) to afford trans-ethyl 5-(5-isopropyl-1,3,4-oxadiazol-2-yl)piperidine-2-carboxylate (0.21 g, 45%) as a brown oil: ¹H NMR (400 MHz, CDCl₃) δ 1.29 (t, J=7.2 Hz, 3H), 1.37 (d, J=6.8 Hz, 6H), 1.54-1.64 (m, 1H), 1.75-1.86 (m, 1H), 2.16-2.21 (m, 1H), 2.28-2.35 (m, 1H), 2.90 (t, J=11.6 Hz, 1H), 2.99 (tt, J=4.0, 11.2 Hz, 1H-axial CH-oxadiazole), 3.12-3.20 (m, 2H), 3.39 (dd, J=3.6, 11.6 Hz, 1H-axial CHCO₂Et), 3.51 (d, J=11.6 Hz, 1H), 4.21 (q, J=7.6, 14.8 Hz, 2H); m/z (APCI pos) 268.1 (100%) (M+H).

### Example F1

### N-(2-(3-Methyl-3-(2-(pyridin-4-yl)ethyl)ureido)ethyl)-1-phenyl-3-(trifluoromethyl)-1H-pyrazole-4-carboxamide

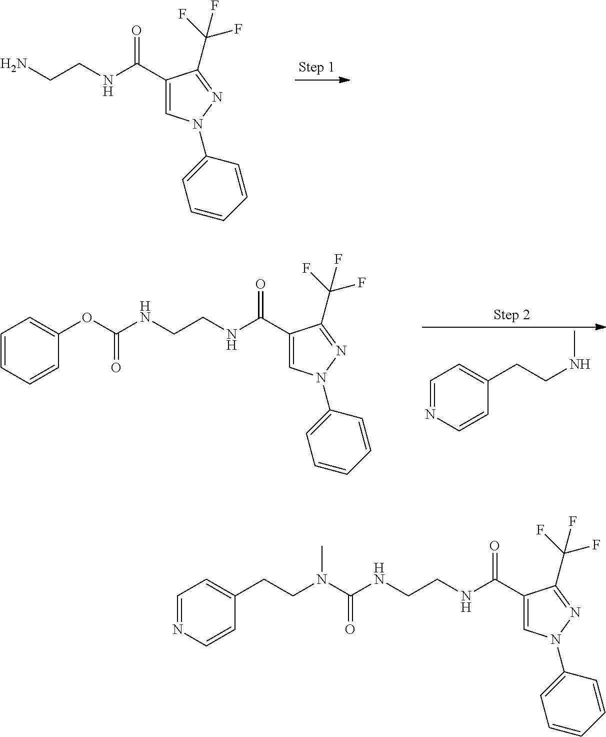

### Step 1

To N-(2-aminoethyl)-1-phenyl-3-(trifluoromethyl)-1H-pyrazole-4-carboxamide (7 g, 23 mmol) and triethylamine (4.7 g, 47 mmol) in DCM (200 mL) at 0° C., was added dropwise phenyl chloroformate (4.4 g, 28 mmol) in DCM (50 mL). The mixture was stirred at room temperature for 2 hours then concentrated to dryness. The residue was purified by MPLC (DCM/MeOH 99/1) to yield phenyl 2-(1-phenyl-3-(trifluoromethyl)-1H-pyrazole-4-carboxamido)ethylcarbamate as a white solid (8.2 g, 84%). Crystallized from AcOEt/hexane: ¹H NMR (400 MHz, DMSO-d₆) 3.20-3.28 (m, 2H), 3.34-3.42 (m, 2H), 7.10 (m, 2H), 7.19 (m, 1H), 7.36 (m, 2H), 7.48 (m, 1H), 7.61 (m, 2H), 7.80 (m, 2H), 7.85 (t, 1H, J=5.9 Hz), 8.47 (t, 1H, J=5.9 Hz), 9.04 (s, 1H); m/e (APCI pos) 419.0 (20%), 299.0 (100%) [M+H].

### Step 2

To phenyl 2-(1-phenyl-3-(trifluoromethyl)-1H-pyrazole-4-carboxamido)ethylcarbamate (200 mg, 0.48 mmol) in EtOH (0.5 mL) was added N-methyl-2-(pyridin-4-yl)ethanamine (65 mg, 0.48 mmol) and the mixture was heated to 140° C. for 30 minutes in an opened vessel. After cooling down, the crude mixture was purified by MPLC (DCM/MeOH 100/0->90/10) to yield N-(2-(3-methyl-3-(2-(pyridin-4-ylethyl)ureido)ethyl)-1-phenyl-3-(trifluoromethyl)-1H-pyrazole-4-carboxamide as a white solid (157 mg, 65%). Crystallized from AcOEt/hexanes: ¹H NMR (400 MHz, DMSO-d₆) δ 2.74 (m, 5H), 3.17 (m, 2H), 3.28 (m, 2H), 3.43 (m, 2H), 6.46 (m, 1H), 7.22 (m, 2H), 7.47 (m, 1H), 7.60 (m, 2H), 7.80 (m, 2H), 8.40 (m, 3H), 9.05 (s, 1H); m/z (APCI pos) 461.1 (100%) [M+H].

### Example F2

### 1-(trans-4-(5-Isopropyl-1,3,4-oxadiazol-2-yl)cyclohexyl)-3-(2-(1-phenyl-3-(trifluoromethyl)-1H-pyrazole-4-carboxamido)ethyl)urea

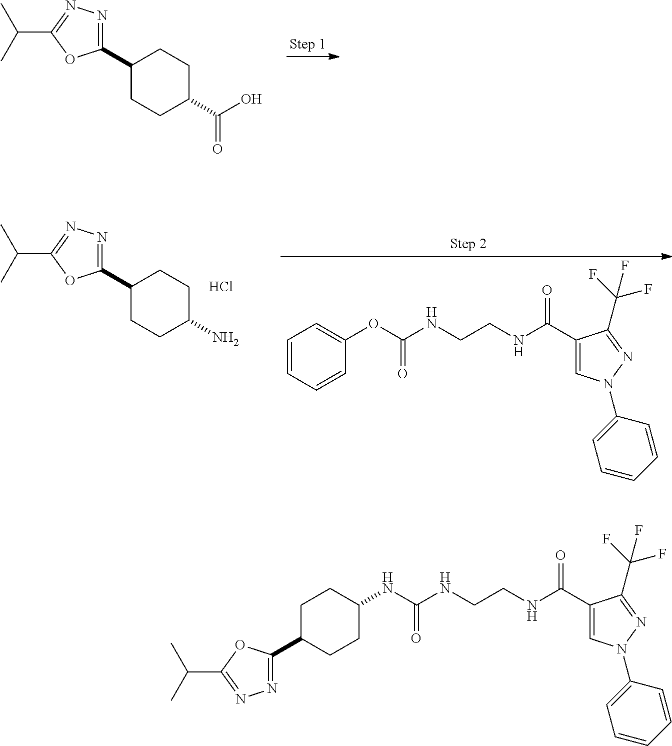

### Step 1

A mixture of trans-4-(5-isopropyl-1,3,4-oxadiazol-2-yl)cyclohexanecarboxylic acid (1.80 g, 7.55 mmol), diphenyl phosphoryl azide (1.79 mL, 8.31 mmol) and triethylamine (1.16 mL, 8.31 mmol) in tert-butanol (20 mL) was heated at 90° C. for 2 days. After cooling to room temperature, the mixture was concentrated in vacuo. The residue was purified by silica gel column chromatography (hexanes/AcOEt 2/1->1/1) to give tert-butyl trans-4-(5-isopropyl-1,3,4-oxadiazol-2-yl)cyclohexylcarbamate (0.88 g, 38%) as a colorless solid. A mixture of the solid (0.88 g, 2.80 mmol) and 4N hydrochloride in dioxane (7.1 ml) was stirred for 16 h at room temperature. The mixture was concentrated in vacuo and dried to give trans-4-(5-isopropyl-1,3,4-oxadiazol-2-yl)cyclohexanamine hydrochloride (0.70 g, 100%) as a colorless solid.

¹H NMR (400 MHz, DMSO-d₆) δ 1.28 (6H, d, J=7.2 Hz), 1.38-1.64 (4H, m), 1.98-2.16 (4H, m), 2.87 (1H, m), 3.06 (1H, m), 3.15 (1H, m), 8.05 (2H, s); m/e (APCI pos) 210.0 (100%) (M+H—HCl).

### Step 2

A mixture of trans-4-(5-isopropyl-1,3,4-oxadiazol-2-yl)cyclohexanamine hydrochloride (0.10 g, 0.41 mmol), phenyl 2-(1-phenyl-3-(trifluoromethyl)-1H-pyrazole-4-carboxamido)ethylcarbamate (0.26 g, 0.61 mmol) and cesium carbonate (0.27 g, 0.81 mmol) in EtOH (5 mL) was heated at 160° C. for 30 min in an open vessel. After cooling to room temperature, the mixture was diluted with AcOEt, washed with water and brine, and the extract was dried and concentrated in vacuo. The residue was purified by preparative HPLC to give 1-(trans-4-(5-isopropyl-1,3,4-oxadiazol-2-yl)cyclohexyl)-3-(2-(1-phenyl-3-(trifluoromethyl)-1H-pyrazole-4-carboxamido)ethyl)urea. Recrystallization from hexanes/AcOEt gave the product (0.031 g, 14%) as yellow crystals.

¹H NMR (400 MHz, CDCl₃) δ 1.16-1.32 (2H, m), 1.37 (6H, d, J=7.2 Hz), 1.54-1.72 (2H, m), 2.04-2.18 (4H, m), 2.79 (1H, m), 3.14 (1H, m), 3.38-3.64 (5H, m), 4.69 (1H, d, J=7.6 Hz), 5.26 (1H, t, J=5.2 Hz), 7.34-7.46 (2H, m), 7.51 (2H, t, J=7.6 Hz), 7.71 (2H, d, J=7.6 Hz), 8.49 (1H, s); m/z (APCI pos) 534.1 (100%) (M+H).

### Formulation Example 1

### Production of Capsules

| | |
|---|---|
| 1) compound of Example E1 | 30 mg |
| 2) fine cellulose powder | 10 mg |
| 3) lactose | 19 mg |
| 4) magnesium stearate | 1 mg |
| total | 60 mg |

1), 2), 3) and 4) are mixed and filled in gelatin capsules.

### Formulation Example 2

### Production of Tablets

| | |
|---|---|
| 1) compound of Example E1 | 30 g |
| 2) lactose | 50 g |
| 3) corn starch | 15 g |
| 4) carboxymethylcellulose calcium | 44 g |
| 5) magnesium stearate | 1 g |
| total of 1000 tablets | 140 g |

The entire amounts of 1), 2) and 3), and 30 g of 4) are kneaded with water, dried in vacuo and granulated.

The granules are mixed with 14 g of 4) and 1 g of 5) and the mixture is compressed with a tableting machine, whereby 1000 tablets containing 30 mg of compound of Example E1 per tablet are obtained.

### Formulation Example 3

### Production of Capsules

| | |
|---|---|
| 1) compound of Example F1 | 30 mg |
| 2) fine cellulose powder | 10 mg |
| 3) lactose | 19 mg |
| 4) magnesium stearate | 1 mg |
| total | 60 mg |

1), 2), 3) and 4) are mixed and filled in gelatin capsules.

### Formulation Example 4

### Production of Tablets

| | |
|---|---|
| 1) compound of Example F1 | 30 g |
| 2) lactose | 50 g |
| 3) corn starch | 15 g |
| 4) carboxymethylcellulose calcium | 44 g |
| 5) magnesium stearate | 1 g |
| total of 1000 tablets | 140 g |

The entire amounts of 1), 2) and 3), and 30 g of 4) are kneaded with water, dried in vacuo and granulated.

The granules are mixed with 14 g of 4) and 1 g of 5) and the mixture is compressed with a tableting machine, whereby 1000 tablets containing 30 mg of compound of Example F1 per tablet are obtained.

### Experimental Example 1

The genetic engineering described below followed the method described in a book (Maniatis et al., Molecular Cloning, Cold Spring Harbor Laboratory (1989)) or a method described in the protocol attached to the reagents.

### (1) Cloning of Human DGAT1 Gene and Preparation of Recombinant Baculovirus

Human DGAT1 gene was cloned by PCR using human adipocyte cDNA (Clontech, QUICK-Clone cDNA, human fat cell, cat #637220) as a template and, based on the DGAT1 gene information reported by Case, S. et al. (Proc. Natl. Acad. Sci. U.S.A. 95 (22), 13018-13023 (1998)), a nucleotide sequence (245-1711 of Genbank Accession No. NM_{—}012079) encoding DGAT1 was amplified with the following PCR primer. The primer nucleotide sequence is shown below.

DGAT1-U:
(SEQ ID NO: 1)
5′ AATTAAGAATTCATGGGCGACTACAAAGACGATGACGACGGCGAC
CGCGGCAGCTCCCGGCGCCGG 3′,
and
DGAT2-L:
(SEQ ID NO: 2)
5′ AATTAAACTAGTTCAGGCCTCTGCCGCTGGGGCCTCATAGTTG
AG 3′

The PCR reaction was conducted using a KOD-plus kit (TOYOBO). The obtained PCR product was electrophoresed on agarose gel (1%), the DNA fragment amplified by PCR was recovered from the gel, and then digested with restriction enzymes EcoRI and SpeI. The DNA treated with the restriction enzymes was electrophoresed on agarose gel (1%), and the obtained DNA fragment was recovered and ligated with plasmid pFASTBAC1 (Invitrogen) digested with restriction enzymes EcoRI and SpeI to give expression plasmid pFB-DGAT1. The nucleotide sequence of the inserted fragment was confirmed and found to be identical with the nucleotide sequence of DGAT1 (245-1711 of Genbank Accession No. NM_{—}012079). Furthermore, using BAC-TO-BAC Baculovirus Expression System (Invitrogen), recombinant baculovirus BAC-DGAT1 was prepared.

### (2) Preparation of Microsome of Sf9 Insect Cells Highly Expressing DGAT1 Enzyme

SF9 cells were sown at 1×10⁶ cells/ml on Sf-900II SFM medium (1 L, Invitrogen) containing 10% fetal calf serum (Trace), 50 mg/L gentamicin (Invitrogen) and 0.1% Pluronic F-68 (Invitrogen), and shaking culture was performed using a 2 L volume Erlenmeyer flask at 27° C., 100 rpm. After culturing for 24 hrs, recombinant baculovirus BAC-DGAT1 (6.7 mL) was added, and the mixture was further cultured for 3 days. The culture medium was centrifuged at 2,000 rpm for 5 min to give virus-infected cells. The infected cells were washed with a phosphate buffered saline (Invitrogen), centrifuged under the same conditions, and the cells were preserved at −80° C. The cryopreserved cells were thawed in ice, suspended in buffer A (50 mM Tris buffer (30 mL, pH 7.4) containing 20% glycerol, 0.15 M NaCl) supplemented with Complete Protease Inhibitor (Boehringer), and ruptured 3 times with a Polytron homogenizer (Kinematica) at 20,000 rpm for 30 sec. The Sf9 microsome fractions were obtained by a conventional method and cryopreserved at −80° C. as a DGAT1 high expression Sf9 microsome.

### (3) Determination of DGAT Inhibitory Activity

As a DGAT1 reaction buffer, a solution having a composition of 100 mM Tris-HCl (pH 7.5), 250 mM sucrose, 150 mM MgCl₂, 0.01% bovine serum albumin (BSA) was used. Using this buffer, a given concentration of the test compound and a composition (100 μl) of 25 μM dioleoylglycerol, 25 μM [¹⁴C]-Oleoyl-CoA, 5 μg protein/ml DGAT1 high expression Sf9 microsome, and 1% acetone were subjected to a triglyceride synthesis reaction at 32° C. for 20 min. A mixture of 300 μL of chloroform:methanol (=1:2) was added to the reaction mixture to quench the reaction. The reaction mixture was sufficiently mixed and distilled water (200 μL) was added to partition the mixture between a chloroform layer (lower layer) and an aqueous layer (upper layer). The chloroform layer (50 μL) was spotted on a thin layer chromatography silica gel plate (TLC plate, Merck) and developed with a solvent (n-hexane:diethyl ether:ethyl acetate:acetic acid=255:30:15:0.6). The developed TLC plate was dried, contacted with a BAS imaging plate (manufactured by FUJIFILM) and measured with BAS2500 (manufactured by FUJIFILM) 16 hr later to numerically show the amount of [¹⁴C]-triglyceride (TG amount) produced during the reaction. The inhibitory rate was calculated by the following formula:

Inhibitory rate (%)=(1−(TG amount with addition of test compound−blank TG amount)/(control TG amount−blank TG amount))×100

The count of the triglyceride produced in the solution reacted without addition of the compound was used as a “control TG amount”, and the count of the triglyceride produced in the solution reacted without addition of the test compound and DGAT1 high expression Sf9 microsome was used as a “blank TG amount”. In addition, the concentration (IC₅₀) of the test compound necessary for inhibiting the triglyceride synthesis by 50% was calculated by PRISM 3.02 (manufactured by GraphPad Software). The inhibitory activity is shown in Table 1.

The inhibitory activity is shown by A<0.01 μM≦B<0.1 μM≦C<1 μM≦D<10 μM according to IC₅₀.

TABLE 1
DGAT inhibitory activity

| Example No. | hDGAT1 IC₅₀ |
|---|---|
| E15 | B |
| E34 | B |
| E63 | C |
| E64 | B |
| E65 | B |
| E74 | B |
| E91 | C |
| E95 | C |
| E101 | B |
| E105 | A |
| E114 | C |
| E126 | B |
| E150 | A |
| E151 | B |
| E163 | B |
| E164 | B |
| E165 | C |
| E172 | C |
| E175 | B |
| E179 | B |
| E189 | B |
| E193 | D |
| E197 | B |
| E207 | C |
| E214 | B |
| E221 | B |
| E225 | C |
| E228 | B |
| E229 | C |
| E234 | C |
| E242 | B |
| E257 | B |
| E260 | B |
| E265 | B |
| E267 | C |
| F1 | D |

### INDUSTRIAL APPLICABILITY

The compound of the present invention has a DGAT inhibitory activity and is useful for the prophylaxis, treatment or improvement of DGAT-related diseases.

The references cited herein, including patents and patent applications, are hereby incorporated in full by reference, to the extent that they have been disclosed herein.

It must be noted that as used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs.

This application is based on application No. 60/832,113 filed in USA, the contents of which are incorporated hereinto by reference.

1. A compound represented by the formula (Ie): 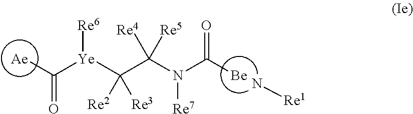
wherein
ring Be is a 5-membered nitrogen-containing aromatic heterocycle optionally condensed with an aromatic ring, which is optionally further substituted;
Re¹ is a substituent;
Ye is CH or N;
ring Ae is an optionally substituted non-aromatic ring; and
Re², Re³, Re⁴, Re⁵, Re⁶ and Re⁷ are each independently a hydrogen atom or a substituent, or any two of Re², Re³, Re⁴, Re⁵, Re⁶ and Re⁷ are optionally bonded to each other to form a non-aromatic ring,
provided that
1) when ring Be is imidazole which is optionally further substituted, then ring Be does not have optionally substituted quinolyl, as a substituent other than Re¹;
2) ring Ae does not have optionally substituted propenoyl as a substituent;
3) when ring Be is pyrrol-2-yl, imidazol-2-yl or pyrazol-5-yl, each of which is optionally further substituted, then Re¹ is an optionally substituted aromatic group;
4) when ring Be is pyrazol-3-yl or pyrazol-4-yl, each of which is optionally further substituted, then Re¹ is not optionally substituted quinolyl; and
5) when ring Be is indole which is optionally further substituted and Ye is CH, then Re¹ is an optionally substituted aromatic group,
or a salt thereof.

2. The compound of claim 1, wherein ring Be is pyrazole, benzimidazole, indole or indazole, each of which is optionally further substituted.

3. The compound of claim 1, wherein Re¹ is an optionally substituted monocyclic aromatic group.

4. The compound of claim 1, wherein ring Ae is a non-aromatic ring optionally substituted by 1 to 3 substituents selected from the group consisting of an optionally substituted hydrocarbon group, an optionally substituted heterocyclic group, an optionally substituted hydroxy group, an optionally substituted amino group, an optionally substituted mercapto group, a cyano group, an oxo group, a halogen atom, —CORe^{a1}, —CO—ORe^{a2}, —SO₂Re^{a2} and —CO—NRe^{a′}Re^{b′}
wherein
Re^{a1} is a hydrogen atom, an optionally substituted C₁₋₁₀ alkyl group, an optionally substituted C₃₋₁₀ cycloalkyl group, an optionally substituted C₆₋₁₄ aryl group or an optionally substituted heterocyclic group;
Re^{a2} is a hydrogen atom, an optionally substituted hydrocarbon group or an optionally substituted heterocyclic group; and
Re^{a}′ and Re^{b}′ are each independently a hydrogen atom, an optionally substituted hydrocarbon group or an optionally substituted heterocyclic group, or Re^{a}′ and Re^{b}′ optionally form, together with the adjacent nitrogen atom, an optionally substituted nitrogen-containing heterocycle.

5. The compound of claim 1, wherein all of Re², Re³, Re⁴, Re⁵, Re⁶ and Re⁷ are hydrogen atoms.

6. A compound represented by the formula (If): 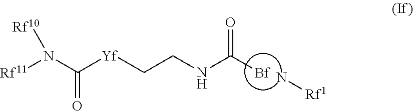
wherein
ring Bf is a 5-membered nitrogen-containing aromatic heterocycle optionally condensed with an aromatic ring, which is optionally further substituted;
Yf is CH₂ or NH;
Rf¹ is a substituent;
Rf¹⁰ is a hydrogen atom or a substituent; and
Rf¹¹ is a hydrogen atom or a C₁₋₆ alkyl group,
provided that
1) when ring Bf is imidazole which is optionally further substituted, then ring Bf does not have optionally substituted quinolyl, as a substituent other than Rf¹;
2) when Yf is CH₂, then ring Bf is not pyrrol-2-yl and imidazol-2-yl, each of which is optionally further substituted;
3) a compound wherein Yf is CH₂, and Rf¹⁰ and Rf¹¹ are hydrogen atoms is excluded;
4) when ring Bf is pyrazol-5-yl which is optionally further substituted, then Rf¹ is an optionally substituted aromatic group; and
5) when ring Bf is pyrazol-3-yl or pyrazol-4-yl, each of which is optionally further substituted, then Rf¹ is not optionally substituted quinolyl,
or a salt thereof.

7. The compound of claim 6, wherein ring Bf is pyrazole, benzimidazole, indole or indazole, each of which is optionally further substituted.

8. The compound of claim 6, wherein RF¹ is an optionally substituted C₆₋₁₄ aryl group or an optionally substituted aromatic heterocyclic group.

9. The compound of claim 6, wherein Rf¹⁰ is an optionally substituted hydrocarbon group.

10. N-(2-(1-phenyl-3-(trifluoromethyl)-1H-pyrazole-4-carboxamido)ethyl)-4-propylpiperidine-1-carboxamide;
N-(2-(1-phenyl-3-(trifluoromethyl)-1H-pyrazole-4-carboxamido)ethyl)-4-(pyrrolidine-1-carbonyl)piperidine-1-carboxamide;
N-(2-(1-phenyl-3-(trifluoromethyl)-1H-pyrazole-4-carboxamido)ethyl)-1-(pyridin-3-ylsulfonyl)piperidine-4-carboxamide;
1-phenyl-N-(2-(4-phenylcyclohexanecarboxamido)ethyl)-3-(trifluoromethyl)-1H-pyrazole-4-carboxamide;
trans-N1-isobutyl-N1-methyl-N4-(2-(1-phenyl-3-(trifluoromethyl)-1H-pyrazole-4-carboxamido)ethyl)cyclohexane-1,4-dicarboxamide;
N-(2-(trans-4-benzoylcyclohexanecarboxamido)ethyl)-1-(pyridin-2-yl)-3-(trifluoromethyl)-1H-pyrazole-4-carboxamide;
N-(2-(trans-4-(5-isopropyl-1,3,4-oxadiazol-2-yl)cyclohexanecarboxamido)ethyl)-1-phenyl-3-(trifluoromethyl)-1H-pyrazole-4-carboxamide;
N-(2-(trans-4-benzoylcyclohexanecarboxamido)ethyl)-1-(2-chlorophenyl)-3-(trifluoromethyl)-1 H-pyrazole-4-carboxamide;
4-(5-isopropyl-1,2,4-oxadiazol-3-yl)-N-(2-(1-phenyl-3-(trifluoromethyl)-1H-pyrazole-4-carboxamido)ethyl)piperidine-1-carboxamide;
N-(2-(trans-4-(5-isopropyl-1,2,4-oxadiazol-3-yl)-cyclohexanecarboxamido)ethyl)-1-phenyl-3-(trifluoromethyl)-1H-pyrazole-4-carboxamide;
2-isopropyl-N-(2-(1-phenyl-3-(trifluoromethyl)-1H-pyrazole-4-carboxamido)ethyl)-4,5,6,7-tetrahydrobenzothiazol-6-carboxamide;
1-(2-chlorophenyl)-N-(2-(trans-4-(5-isopropyl-1,2,4-oxadiazol-3-yl)cyclohexanecarboxamido)ethyl)-3-(trifluoromethyl)-1H-pyrazole-4-carboxamide;
1-(2-fluorophenyl)-N-(2-(trans-4-(5-isopropyl-1,2,4-oxadiazol-3-yl)cyclohexanecarboxamido)ethyl)-3-(trifluoromethyl)-1H-pyrazole-4-carboxamide;
1-(2-fluorophenyl)-N-(2-(trans-4-(5-isopropyl-1,3,4-oxadiazol-2-yl)cyclohexanecarboxamido)ethyl)-1H-indole-3-carboxamide; or
trans-5-(5-isopropyl-1,3,4-oxadiazol-2-yl)-1-methyl-N-(2-(1-phenyl-3-(trifluoromethyl)-1H-pyrazole-4-carboxamido)ethyl)piperidine-2-carboxamide;
or a salt thereof.

11. A prodrug of the compound of claim 1 or 6.

12. A pharmaceutical agent comprising the compound of claim 1 or 6, or a prodrug thereof.

13. The pharmaceutical agent of claim 12, which is an agent for the prophylaxis or treatment of obesity, hyperlipidemia or diabetes.

14. A DGAT inhibitor comprising the compound of claim 1 or 6, or a prodrug thereof.

15-16. (canceled)

17. A method for the prophylaxis or treatment of obesity, hyperlipidemia or diabetes in a mammal, which comprises administering the compound of claim 1 or 6, or a prodrug thereof to the mammal.

18. A method of inhibiting DGAT in a mammal, which comprises administering the compound of claim 1 or 6, or a prodrug thereof to the mammal.


SEQUENCE LISTING (2 sequences)

SEQ ID NO: 1 - DNA, 66 bp, Artificial
  primer for cloning human DGAT1-U gene
aattaagaattcatgggcgactacaaagacgatgacgacggcgaccgcggcagctcccggcgccgg

SEQ ID NO: 2 - DNA, 45 bp, Artificial
  primer for cloning human DGAT2-L gene
aattaaactagttcaggcctctgccgctggggcctcatagttgag